(19) United States
(12) Patent Application Publication
(10) Pub. No.: US 2024/0425491 A1
(43) Pub. Date: Dec. 26, 2024

(54) HETEROARYL COMPOUNDS USEFUL IN THE TREATMENT OF COGNITIVE DISORDERS

(71) Applicant: University College Cardiff Consultants Limited, Cardiff (GB)

(72) Inventors: Simon WARD, Cardiff (GB); John ATACK, Cardiff (GB); Alexander ASHALL-KELLY, Cardiff (GB); Alex BALDWIN, Cardiff (GB); David FOLEY, Cardiff (GB); Heulyn JONES, Cardiff (GB); Wai Leung YU, Cardiff (GB); Stephen BRAND, Alyth (GB); Srinivasan NATARAJAN, Ashford (GB)

(21) Appl. No.: 18/288,711

(22) PCT Filed: May 4, 2022
(86) PCT No.: PCT/GB2022/051132

§ 371 Date: Oct. 27, 2023

(30) Foreign Application Priority Data
May 5, 2021 (GB) 2106385.4
Feb. 15, 2022 (GB) 2201949.1

(51) Int. Cl. C07D 413/14; A61K 31/4178; A61K 31/4192; A61K 31/4439; A61K 31/495; A61K 31/4985; C07D 403/12; C07D 413/12; C07D 417/14; C07D 487/04; C07D 493/04; C07D 493/08; C07D 493/10; C07D 498/04

(52) U.S. Cl. CPC C07D 413/14; A61K 31/4178; A61K 31/4192; A61K 31/4439; A61K 31/495; A61K 31/4985; C07D 403/12; C07D 413/12; C07D 417/14; C07D 487/04; C07D 493/04; C07D 493/08; C07D 493/10; C07D 498/04

(57) ABSTRACT

The present invention provides compounds of Formula (I), as well as pharmaceutically acceptable salts thereof: wherein X¹, X², X³, R³, Ring A and Ring B are as described herein. The compounds have affinity for α5-subunit-containing GABA_{A} receptors. The invention further provides the manufacture of the compounds of formula (I), pharmaceutical compositions comprising the compounds and their use as medicaments for the treatment of diseases and disorders associated with α5-GABA_{A} receptors, including depression and cognitive impairment, for example, cognitive impairment associated with a psychotic disorder such as schizophrenia.

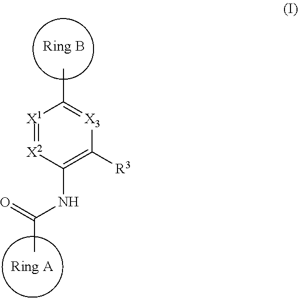

The invention relates to compounds that are γ-aminobutyric acid (GABA) type A receptor (GABA_{A}R) negative allosteric modulators (NAMs), in particular those that are selective for α5-subunit-containing GABA_{A}Rs (α5-GABA_{A}Rs), their manufacture, pharmaceutical compositions comprising the compounds and their use as medicaments. The compounds of the invention are useful in the treatment of diseases and medical conditions associated with α5-GABA_{A}Rs, including, for example, cognitive disorders, Alzheimer's disease or Huntington's disease.

### BACKGROUND

In the mammalian central nervous system, GABA is the main inhibitory neurotransmitter. Receptors for this neurotransmitter are divided into three main classes: the type A and C receptors (GABA_{A}Rs and GABA_{C}Rs), which are related members of the ligand-gated ion channel superfamily (and which are also frequently collectively called GABARs with no reference to the pharmacology that differentiates GABA_{A}Rs and GABA_{C}Rs), and the type B receptors (GABA_{B}Rs), which are members of the G-protein coupled receptor family.

GABA_{A}Rs are membrane bound heteropentamers made up from 19 known subunits. Most GABA_{A}Rs contain two a subunits, two β subunits and one γ subunit with two GABA binding sites formed at the interface of a and β subunits. Although there are a very large number of theoretical heteropentameric assemblies of the different GABA_{A}R proteins, there are only ˜25 thought to exist in the brain (Olsen R. W. and Sieghart W. 2008 Pharmacology Review 60 243-260). The pharmacology of GABA_{A}Rs can be defined in terms of their sensitivity (or not) to benzodiazepines which act as positive allosteric modulators (PAMs) at GABA_{A}Rs comprising β and γ2 subunits in conjunction with either an α1, α2, α3 or α5 (but not α4 or α6) subunit. These different α-containing GABA_{A}Rs mediate distinct physiological and pharmacological functions and can have different expression levels in various parts of the body.

GABA_{A}R PAMs and negative allosteric modulators (NAMs) acting at the benzodiazepine recognition site have opposite pharmacological effects at the molecular and whole animal level and in preclinical species and man (Atack J. 2011 Current Topics in Medicinal Chemistry 11 1176-1202; Atack J. 2011 Current Topics in Medicinal Chemistry 11 1203-1214). For example, there is evidence to show that non-selective GABA_{A}R PAMs, exemplified by the benzodiazepine diazepam, are typically anxiolytic, sedating, anticonvulsant and impair cognition (Atack J. 2011 Current Topics in Medicinal Chemistry 11 1176-1202), whereas non-selective GABA_{A}R NAMs are anxiogenic, proconvulsant and enhance cognition with, for instance, the B-carboline FG7142 being anxiogenic and producing a proconvulsant-like aura in normal volunteers (Dorrow, R. et al. 1983 Lancet 2 98-99). On the other hand, α5-GABA_{A}R NAMs such as α5IA, MRK016 and RG1662 have no proconvulsant-like side effects and neither are they anxiogenic in man (Atack, J. 2010 Pharmacology & Therapeutics 125 11-26).

Although α5-GABA_{A}Rs are a relative minority of the total brain GABA_{A}R population, they are highly expressed in the hippocampus where they are presumably associated with cognition and other hippocampal functions. α5-GABA_{A}Rs have been implicated in the pathophysiology and treatment of various diseases including: Down's syndrome (Martínez-Cué C. et al. 2013 Journal of Neuroscience 33 3953-3966), depression (Zanos P. et al. 2017 eNeuro 4 ENEURO.0285-16.2017), anaesthetic induced cognitive dysfunction (Zurek A. A. et al. 2014 Journal of Clinical Investigation 124 5437-5441), stroke (Gacsályi, I. 2018 European Journal of Pharmacology 834 118-125), mild cognitive impairment and/or Alzheimer's disease (Atack J. et al. 2009 Journal of Pharmacology and Experimental Therapeutics 331 470-484; Ballard T. et al. 2009 Psychopharmacology 202 207-223; Kawaharada S. et al. 2018 Journal of Pharmacology and Experimental Therapeutics 366 58-65), alcohol-related disorders (Platt D. 2005 et al. Journal of Pharmacology and Experimental Therapeutics 313 658-667) and impaired brain function related to neuroinflammation (Eimerbrink M. et al. 2015 Behavioural Brain Research, 288 50-53), or bacterial or viral infections such as HIV (Green and Thayer 2019 Neuropharmacology 149 161-168, and Jacob, 2019 Frontiers in Molecular Neuroscience, 12 179).

α5-GABA_{A}R modulators have entered into clinical trials for various indications including Down syndrome (RG1662/basmisanil, NCT02024789), schizophrenia (RG1662/basmisanil, NCT02953639), stroke (S44819, NCT02877615) and Dup15q syndrome (NCT05307679).

Certain α5-GABA_{A}R modulators are described in WO 98/50385 (Merck Sharp & Dohme; WO2018/104419, WO2012/062687, WO2010/127978, WO2010/127976, WO2010/127974, WO2010/112475, WO2010/097368, WO2010/094669, WO 2009/071476, WO2009/071477, WO2009/071476, WO2009/071464 (Hoffmann-La Roche); WO 2015/115673 (Ono Pharmaceuticals); WO 2014/001279 (Saniona); WO2019/046300 (University of Maryland); and WO 2022/029170.

There remains a need for new α5-GABA_{A}R modulators, particularly α5-GABA_{A}R NAMs.

### BRIEF SUMMARY OF THE DISCLOSURE

In accordance with the present inventions there is provided a compound of the formula (I), or a pharmaceutically acceptable salt thereof:

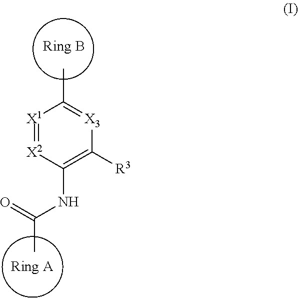

wherein
Ring A is selected from: A1, A2 and A3:

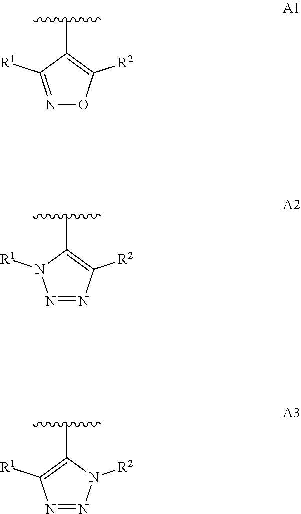

R¹ is selected from: phenyl and a 5- or 6-membered heteroaryl wherein R¹ is optionally substituted by one or more substituents selected from: halo, C₁₋₄ alkyl, C₁₋₄ haloalkyl, —OR^{a1}, —SR^{a1} and —NR^{a1}R^{b1};
R² is selected from: H, halo, C₁₋₄ alkyl and C₁₋₄ haloalkyl, —OR^{a2}, —SR^{a2} and —NR^{a2}R^{b2}
   wherein the C₁₋₄ alkyl is optionally substituted by one or more substituents selected from: halo, —OR^{a3}, —SR^{a3} and —NR^{a3}R^{b3};
R³ is selected from: C₁₋₄ alkyl, C₁₋₄ haloalkyl, C₃₋₆ cycloalkyl, C₃₋₆ cycloalkyl-C₁₋₃ alkyl-, —OR⁴, —NR⁵R⁶, —SR⁵, 4- to 7-membered heterocyclyl containing 1 or more ring oxygen atoms, and 4- to 7-membered heterocyclyl-C₁₋₃ alkyl-containing 1 or more ring oxygen atoms;
   R⁴ and R⁵ are independently selected from: H, C₁₋₄ alkyl, C₁₋₄ haloalkyl, C₃₋₆ cycloalkyl, C₃₋₆ cycloalkyl-C₁₋₃ alkyl-, 4- to 7-membered heterocyclyl containing 1 or more ring oxygen atoms, and 4- to 7-membered heterocyclyl-C₁₋₃ alkyl-containing 1 or more ring oxygen atoms;
   R⁶ is selected from: H, C₁₋₄ alkyl and C₁₋₄ haloalkyl;
   wherein any C₁₋₄ alkyl, C₃₋₆ cycloalkyl or C₃₋₆ cycloalkyl-C₁₋₃ alkyl- in any of R³, R⁴, R⁵ or Re is optionally substituted by one or more substituents selected from: halo, —OR^{a4}, —SR^{a4} and —NR^{a4}R^{b4};
X¹, X² and X³ are independently selected from: N and CR⁷;
   R⁷ is at each occurrence independently selected from: H, halo, —CN, C₁₋₄ alkyl, C₁₋₄ haloalkyl, —OR⁸, —NR⁸R⁹ and —S(O)ₓR⁸ (wherein x is 0, 1, or 2);
   R⁸ and R⁹ are each independently selected from: H, C₁₋₄ alkyl and C₁₋₄ haloalkyl;
   wherein any C₁₋₄ alkyl in any of R⁷, R⁸ or R⁹ is optionally substituted by one or more substituents selected from: halo, —CN, —OR^{a5}, —S(O)ₓR^{a5} (wherein x is 0, 1, or 2) and —NR^{a5}R^{b5};
Ring B is selected from C₆₋₁₀ aryl and 5- to 12-membered heteroaryl optionally substituted with one or more R¹⁰, wherein when Ring B is heteroaryl, Ring B is bonded to the remainder of the compound of Formula (I) by a ring atom in an aromatic ring of the heteroaryl;
   R¹⁰ at each occurrence is independently selected from: halo, —CN, —NO₂, ═O, C₁₋₆ alkyl, C₂₋₆ alkenyl, C₂₋₆ alkynyl, C₁₋₆ haloalkyl, Q¹-L¹-, —OR¹¹, —S(O)₂R¹¹ (wherein x is 0, 1, or 2), —NR¹¹R^{a6}, —C(O)R¹¹, —OC(O)R¹¹, —C(O)OR¹¹, —NR^{a6}C(O)R¹¹, —NR^{a6}C(O)OR¹¹, —C(O)NR¹¹R^{a6}, —OC(O)NR¹¹R^{a6}, —NR^{a6}SO₂R¹¹, —SO₂NR¹¹R^{a6} and —NR^{a6}C(O)NR¹¹R^{a6},
   wherein said C₁₋₆ alkyl, C₂₋₆ alkenyl and C₂₋₆ alkynyl is optionally substituted by 1 or more R¹²;
   R¹¹ is independently selected from: H, C₁₋₆ alkyl and C₁₋₆ haloalkyl, wherein said C₁-6 alkyl is optionally substituted by one or more R¹³;
   Q¹ at each occurrence is independently selected from: C₃₋₆ cycloalkyl, C₃₋₆ cycloalkyl-C₁₋₃ alkyl-, 4- to 7-membered heterocyclyl, 4- to 9-membered heterocyclyl-C₁₋₃ alkyl-, phenyl, phenyl-C₁₋₃ alkyl-, 5- or 6-membered heteroaryl and 5- or 6-membered heteroaryl-C₁₋₃ alkyl-,
   wherein said C₃₋₆ cycloalkyl, C₃₋₆ cycloalkyl-C₁₋₃ alkyl-, 4- to 7-membered heterocyclyl and 4- to 9-membered heterocyclyl-C₁₋₃ alkyl- is optionally substituted by one or more R¹⁴, and
   wherein said phenyl, phenyl-C₁₋₃ alkyl-, 5- or 6-membered heteroaryl and 5- or 6-membered heteroaryl-C₁₋₃ alkyl- is optionally substituted by one or more R¹⁵;
   L¹ is a bond or is selected from —O—, —S(O)ₓ— (wherein x is 0, 1, or 2), —NR^{a7}—, —C(O)—, —OC(O)—, —C(O)O—, —NR^{a7}C(O)—, —C(O)NR^{a7}—, —NR^{a7}C(O)O—, —OC(O)NR^{a7}—, —NR^{a7}SO₂—, —SO₂NR^{a7}— and —NR^{a7}C(O)NR^{a7}—;
   R¹², R¹³ and R¹⁴ are at each occurrence independently selected from: halo, ═O, —CN, —NO₂, C₁₋₄ alkyl, C₁₋₄ haloalkyl, —OR^{a8}, —S(O)₂R^{a8}, —NR^{a8}R^{b8}, —C(O)R^{a8}, —OC(O)R^{a8}, —C(O)OR^{a8}, —NR^{a8}C(O)R^{b8}, —C(O)NR^{a8}R^{b8}, —NR^{a8}C(O)OR^{b8}, —OC(O)NR^{a8}R^{b8}, —NR^{a8}SO₂R^{b8} and —SO₂NR^{a8}R^{b8},
   wherein said C₁₋₄ alkyl is optionally substituted by 1 or 2 substituents selected from: halo, —CN, —OR^{a9}, —NR^{a9}R^{b9} and —SO₂R^{a9};
   R¹⁵ is at each occurrence independently selected from: halo, ═O, —CN, —NO₂, C₁₋₄ alkyl, C₁₋₄ haloalkyl, —OR^{a10}, —S(O)₂R^{a10}, —NR^{a10}R^{b10}, —C(O)R^{a10}, —OC(O)R^{a10}, —C(O)OR^{a10}, —NR^{a10}C(O)R^{b10}, —C(O)NR^{a10}R^{b10}, —NR^{a10}C(O)OR^{b10}, —OC(O)NR^{a10}R^{b10}, —NR^{b10}SO₂R^{a10} and —SO₂NR^{a10}R^{b10};
   wherein said C₁₋₄ alkyl is optionally substituted by 1 or 2 substituents selected from: halo, —CN, —OR^{a11}, —NR^{a11}R^{b11} and —SO₂R^{a11};
R^{a1}, R^{b1}, R^{a2}, R^{b2}, R^{a3}, R^{b3}, R^{a4}, R^{b4}, R^{a5}, R^{b5}, R^{a6}, R^{a7}, R^{a8}, R^{b8}, R^{a9}, R^{b9}, R^{a10}, R^{b10}, R^{a11} and R^{b11} are at each occurrence independently selected from: H, C₁₋₄ alkyl and C₁₋₄ haloalkyl,
   or any —NR^{a1}R^{b1}, —NR^{a2}R^{b2}, —NR^{a3}R^{b3}, —NR^{a4}R^{b4}, —NR^{a5}R^{b5}, —NR^{a8}R^{b8}, —NR^{a9}R^{b9}, —NR^{a10}R^{b10}, —NR^{a11}R^{b11}, —NR⁵R⁶, —NR⁸R⁹ or —NR¹¹R^{a6} within a substituent may form a 4- to 6-membered heterocyclyl, wherein said 4- to 6-membered heterocyclyl is optionally substituted by one or more substituents selected from: halo, ═O, C₁₋₄ alkyl and C₁₋₄ haloalkyl,
   with the provisos (i) and (ii):
   (i) when Ring A is A2, and R² is H, then R³ is not —NR⁵R⁶; and
   (ii) the compound of formula (I) is not:

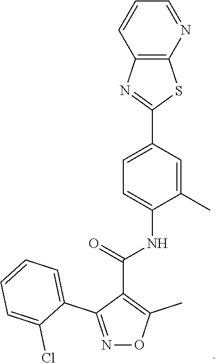 [SMILES: Cc1cc(-c2nc3cccnc3s2)ccc1NC(=O)c1c(-c2ccccc2Cl)noc1C]

Also provided is a pharmaceutical composition comprising a compound of the invention, or a pharmaceutically acceptable salt thereof, and a pharmaceutically acceptable excipient.

Also provided is a compound of the invention, or a pharmaceutically acceptable salt thereof, for use as a medicament. In certain embodiments the compound of the invention, or a pharmaceutically acceptable salt thereof, is for use in the treatment of a disease or medical condition mediated by α5-GABA_{A}Rs.

Also provided is a method of treating a disease or medical condition mediated by α5-GABA_{A}Rs in a subject in need thereof, the method comprising administering to the subject an effective amount of a compound of the invention, or a pharmaceutically acceptable salt thereof.

In certain embodiments there is provided a compound of the invention, or a pharmaceutically acceptable salt thereof, for use in the prevention or treatment of a neurological or neuropsychiatric disorder mediated by α5-GABA_{A}Rs. In certain embodiments there is provided a compound of the invention, or a pharmaceutically acceptable salt thereof, for use in the prevention or treatment of cognitive dysfunction associated with a neurological or neuropsychiatric disorder involving α5-GABA_{A}Rs. It may be that the neurological disorder is a neurodevelopment disorder, for instance Down syndrome or neurodegenerative disorder such as Alzheimer's disease or Huntington's disease. Thus also provided is a compound of the invention, or a pharmaceutically acceptable salt thereof, for use in the treatment of a neurological condition (e.g. Down syndrome). Also provided is a compound of the invention, or a pharmaceutically acceptable salt thereof, for use in the treatment of neurodegenerative disorder (e.g. Alzheimer's disease or Huntington's disease).

It may be that the disease or medical disorder mediated by α5-GABA_{A}Rs is selected from: Alzheimer's disease, Parkinson's disease, Huntington's disease, cognitive dysfunction (e.g. cognitive dysfunction associated with chemotherapy, an anaesthetic, a bacterial infection or a viral infection (e.g. HIV)), memory deficit, age-related cognitive impairment (i.e., mild cognitive impairment, MCI), a bipolar disorder, autism, Down syndrome, neurofibromatosis type I, a sleep disorder, a disorder of circadian rhythms, amyotrophic lateral sclerosis (ALS), a psychotic disorder (for example, schizophrenia, schizoaffective disorder, schizophreniform disorder, substance-induced psychotic disorder or paraphrenia), psychosis, post-traumatic stress disorder, an anxiety disorder, a generalized anxiety disorder, a panic disorder, a delusional disorder, an obsessive/compulsive disorder, an acute stress disorder, drug addiction, alcohol disorders (e.g. alcohol addiction), drug withdrawal symptoms, a movement disorder, restless leg syndrome, a cognition deficiency disorder, multi-infarct dementia, vascular dementia, a mood disorder, depression, a neuropsychiatric condition, attention-deficit/hyperactivity disorder, neuropathic pain, chronic neuroinflammation, cognitive dysfunction associated with stroke, cognitive dysfunction associated with brain injury or trauma, cognitive dysfunction associated with a brain tumour, an attentional disorder, and Dup15q syndrome.

In certain embodiments a compound of the invention, or a pharmaceutically acceptable salt thereof, is for use in the treatment or prevention of depression, for example the treatment of treatment-resistant depression.

In certain embodiments a compound of the invention, or a pharmaceutically acceptable salt thereof, is for use in the treatment or prevention of post-operative cognitive dysfunction in a subject.

In certain embodiments a compound of the invention, or a pharmaceutically acceptable salt thereof, is for use in the treatment or prevention of neuroinflammation-induced psychiatric or neurological symptoms. For example, a compound of the invention, or a pharmaceutically acceptable salt thereof, for use in the treatment of neuroinflammation induced cognitive impairment.

In embodiments a compound of the invention, or a pharmaceutically acceptable salt thereof, is for use in the treatment or prevention of cognitive impairment associated with a bacterial or viral infection in a subject.

### BRIEF DESCRIPTION OF THE DRAWINGS

Embodiments of the invention are further described hereinafter with reference to the accompanying drawings, in which:

FIG. 1 shows raw whole-cell current traces recorded on the QPatch system from the same cell expressing α533γ2 GABA_{A} receptors before and after the addition of the compound described in Example 1 (1 μM) using the in vitro electrophysiological recording assay described herein.

FIG. 2 illustrates the rescue of the etomidate-mediated long-term potentiation (LTP) deficit by the compound of Example 1 in a mouse hippocampal brain slices LTP assay described herein. The Y-axis in FIG. 2A shows the field excitatory postsynaptic potential (fEPSP) slope expressed as a % of the control fEPSPs prior to the 4-theta burst stimulation (4-TBS). The Y-axis in FIG. 2B shows the fEPSP slope at 50 to 60 minutes post delivery of the 4-TBS.

FIG. 3 shows the occupancy of rat brain benzodiazepine (BZ) binding sites by compound Example 1 in the in vivo brain receptor occupancy assay described herein at doses in the range 3 to 30 mg/kg (p.o.).

### DETAILED DESCRIPTION

### Definitions

Unless otherwise stated, the following terms used in the specification and claims have the following meanings set out below.

Reference herein to a “compound of the invention” is a reference to any of the compounds disclosed herein including compounds of the formulae (I) to (XXXI), a compound selected from Compound List 1, or a compound described in any of the Examples, or a pharmaceutically acceptable salt, solvate, or salt of a solvate of any thereof.

Reference to a “α5-GABA_{A}R” refers to a GABA_{A}R that comprises at least one α5 subunit, for example one or two α5 subunits.

The term “negative allosteric modulator” or “NAM” refers to an agent that acts at an allosteric site on α5-GABA_{A}Rs and indirectly reduces the responsiveness of the receptor to the endogenous ligand (GABA).

The terms “treating”, or “treatment” refer to any beneficial effect in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; modifying the progression of a disease or condition, making the final point of degeneration less debilitating; improving a patient's physical or mental well-being. The treatment or amelioration of symptoms can be based on objective or subjective parameters; including the results of a physical examination, neuropsychiatric examinations, and/or a psychiatric evaluation. The term “treating” and conjugations thereof, includes prevention of an injury, pathology, condition, or disease (i.e. prophylaxis or prevention). For example, the term “treating” and conjugations thereof, include prevention of a pathology, condition, or disease associated with α5-GABA_{A}Rs (e.g. reducing or preventing cognitive dysfunction associated with the condition or disease).

The terms “cognitive dysfunction” or “cognitive impairment” refers to deficits in cognitive function as defined in the Diagnostic and Statistical Manual of Mental Disorders (DSM-5). Examples of cognitive dysfunction include deficits in one or more of executive function, learning, memory, perception, problem solving, language, or social cognition. Cognitive dysfunction may be associated with a neurocognitive disorder wherein cognition declines (e.g. as in Alzheimer's disease, Huntington's disease, Parkinson's disease or dementia). Cognitive dysfunction may also be associated with neurodevelopment disorders (e.g. Down syndrome, autism or attention deficit/hyperactivity disorder (ADHD)).

The term “associated” or “associated with”, “involving” or “mediated by” in the context of a α5-GABA_{A}Rs associated with a disease means that the disease is caused by (in whole or in part), or a symptom of the disease is caused by (in whole or in part) by α5-GABA_{A} receptors, or receptor activity or function. For example, a symptom of a disease or condition associated with α5-GABA_{A}R pathway activity may be a symptom that results (entirely or partially) from an increase in the level of activity of α5-GABA_{A}R protein pathways. As used herein, what is described as being associated with a disease, if a causative agent, could be a target for treatment of the disease. For example, a disease associated with an increase in the level of α5-GABA_{A}R activity, may be treated with an agent (e.g. compound as described herein) effective for decreasing the level of activity of α5-GABA_{A}Rs.

An “effective amount” is an amount sufficient to accomplish a stated purpose. For example an amount sufficient to achieve the effect for which it is administered, treat a disease, reduce enzyme activity, increase enzyme activity, reduce receptor signalling, increase receptor signalling, reduce one or more symptoms of a disease or condition, or to provide a disease modifying effect (i.e. alter the underlying pathophysiology of the disease). An example of an “effective amount” is an amount sufficient to contribute to the treatment, prevention, or reduction of a symptom or symptoms of a disease, or modify the progression of a disease, which could also be referred to as a “therapeutically effective amount.” A “reduction” of a symptom or symptoms means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The therapeutically effective amount of a compound of the invention can be initially estimated from cell culture assays. Target concentrations will be those concentrations of active compound(s) that are capable of achieving the therapeutic effect described herein, as measured using the methods described herein or known in the art.

Therapeutically effective amounts for use in humans can also be determined from animal models using known methods. For example, a dose for humans can be formulated to achieve a concentration that has been found to be effective in animals. The dosage in humans can be adjusted by monitoring compound effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated, or in response to a biomarker or other correlate or surrogate end-point of the disease. This will provide a therapeutic regimen that is commensurate with the severity of the individual's disease state.

A prophylactic or therapeutic treatment regimen is suitably one that does not cause substantial toxicity and yet is effective to treat the clinical symptoms demonstrated by the particular patient. This determination of a dosage regimen is generally based upon an assessment of the active compound by considering factors such as compound potency, relative bioavailability, patient body weight, presence and severity of adverse side effects, preferred mode of administration and the toxicity profile of the selected agent.

The term “halo” or “halogen” refers to one of the halogens, group 17 of the periodic table. In particular the term refers to fluorine, chlorine, bromine and iodine. Preferably, the term refers to fluorine or chlorine.

The term Cₘ₋ₙ refers to a group with m to n carbon atoms.

The term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, iso-butyl, sec-butyl, tert-butyl, n-pentyl and n-hexyl. “C₁₋₄ alkyl” similarly refers to such groups containing up to 4 carbon atoms. Alkylene groups are divalent alkyl groups and may likewise be linear or branched and have two points of attachment to the remainder of the molecule. Furthermore, an alkylene group may, for example, correspond to one of those alkyl groups listed in this paragraph. For example, C₁₋₆ alkylene may be —CH₂—, —CH₂CH₂—, —CH₂CH(CH₃)—, —CH₂CH₂CH₂— or —CH₂CH(CH₃) CH₂—. The alkyl and alkylene groups may be unsubstituted or substituted by one or more substituents. Possible substituents are described herein. For example, substituents for an alkyl or alkylene group may be halogen, e.g. fluorine, chlorine, bromine and iodine, OH, C₁-C₄ alkoxy, —NR′R″ amino, wherein R′ and R″ are independently H or alkyl. Other substituents for the alkyl group may alternatively be used.

The term “C₁₋₆ haloalkyl”, e.g. “C₁₋₄ haloalkyl”, refers to a hydrocarbon chain substituted with at least one halogen atom independently chosen at each occurrence, for example fluorine, chlorine, bromine and iodine. The halogen atom may be present at any position on the hydrocarbon chain. For example, C₁₋₆ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. A haloalkyl group may be, for example, —CX₃, —CHX₂, —CH₂CX₃. —CH₂CHX₂ or —CX(CH₃) CH₃ wherein X is a halo (e.g. F, Cl, Br or I). A fluoroalkyl group, i.e. a hydrocarbon chain substituted with at least one fluorine atom (e.g. —CF₃, —CHF₂, —CH₂CF₃ or —CH₂CHF₂).

The term “C₂₋₆ alkenyl” includes a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl. Alkenylene groups are divalent alkenyl groups and may likewise be linear or branched and have two points of attachment to the remainder of the molecule. Furthermore, an alkenylene group may, for example, correspond to one of those alkenyl groups listed in this paragraph. For example alkenylene may be —CH═CH—, —CH₂CH═CH—, —CH(CH₃) CH═CH— or —CH₂CH═CH—. Alkenyl and alkenylene groups may unsubstituted or substituted by one or more substituents.

Possible substituents are described herein. For example, substituents may be those described above as substituents for alkyl groups.

The term “C₂₋₆ alkynyl” includes a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl. Alkynylene groups are divalent alkynyl groups and may likewise be linear or branched and have two points of attachment to the remainder of the molecule. Furthermore, an alkynylene group may, for example, correspond to one of those alkynyl groups listed in this paragraph. For example alkynylene may be —C≡C—, —CH₂C≡C—, —CH₂C≡CCH₂—, —CH(CH₃) CH═C— or —CH₂C≡CCH₃. Alkynyl and alkynylene groups may unsubstituted or substituted by one or more substituents. Possible substituents are described herein. For example, substituents may be those described above as substituents for alkyl groups.

The term “C₃₋₆ cycloalkyl” includes a saturated hydrocarbon ring system containing 3, 4, 5 or 6 carbon atoms. For example, the “C₃-C₆ cycloalkyl” may be cyclopropyl, cyclobutyl, cyclopentyl, cyclohexyl, bicyclo[2.1.1]hexane or bicyclo[1.1.1]pentane. Suitably the “C₃-C₆ cycloalkyl” may be cyclopropyl, cyclobutyl, cyclopentyl or cyclohexyl.

The term “heterocyclyl”, “heterocyclic” or “heterocycle” includes a non-aromatic saturated or partially saturated monocyclic or fused, bridged, or spiro bicyclic heterocyclic ring system. Monocyclic heterocyclic rings may contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles may contain from 7 to 12-member atoms in the ring. Bicyclic heterocyclic(s) rings may be fused, spiro, or bridged ring systems. The heterocyclyl group may be a 3-12, for example, a 3- to 9- (e.g. a 3- to 7-) membered non-aromatic monocyclic or bicyclic saturated or partially saturated group comprising 1, 2 or 3 heteroatoms independently selected from O, S and N in the ring system (in other words 1, 2 or 3 of the atoms forming the ring system are selected from O, S and N). By partially saturated it is meant that the ring may comprise one or two double bonds. This applies particularly to monocyclic rings with from 5 to 7 members. The double bond will typically be between two carbon atoms but may be between a carbon atom and a nitrogen atom. Bicyclic systems may be spiro-fused, i.e. where the rings are linked to each other through a single carbon atom; vicinally fused, i.e. where the rings are linked to each other through two adjacent carbon and/or nitrogen atoms; or they may be share a bridgehead, i.e. the rings are linked to each other through two non-adjacent carbon or nitrogen atoms (a bridged ring system). Examples of heterocyclic groups include cyclic ethers such as oxiranyl, oxetanyl, tetrahydrofuranyl, dioxanyl, and substituted cyclic ethers. Heterocycles comprising at least one nitrogen in a ring position include, for example, azetidinyl, pyrrolidinyl, piperidinyl, piperazinyl, morpholinyl, thiomorpholinyl, tetrahydrotriazinyl, tetrahydropyrazolyl, tetrahydropyridinyl, homopiperidinyl, homopiperazinyl, 2,5-diaza-bicyclo[2.2.1]heptanyl and the like. Typical sulfur containing heterocycles include tetrahydrothienyl, dihydro-1,3-dithiol, tetrahydro-2H-thiopyran, and hexahydrothiepine. Other heterocycles include dihydro oxathiolyl, tetrahydro oxazolyl, tetrahydro-oxadiazolyl, tetrahydrodioxazolyl, tetrahydrooxathiazolyl, hexahydrotriazinyl, tetrahydro oxazinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O), for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. For example, the term “piperidino” or “morpholino” refers to a piperidin-1-yl or morpholin-4-yl ring that is linked via the ring nitrogen.

The term “bridged ring systems” includes ring systems in which two rings share more than two atoms, see for example Advanced Organic Chemistry, by Jerry March, 4th Edition, Wiley Interscience, pages 131-133, 1992. Suitably the bridge is formed between two non-adjacent carbon or nitrogen atoms in the ring system. The bridge connecting the bridgehead atoms may be a bond or comprise one or more atoms. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane, and quinuclidine.

The term “spiro bi-cyclic ring systems” includes ring systems in which two ring systems share one common spiro carbon atom, i.e. the heterocyclic ring is linked to a further carbocyclic or heterocyclic ring through a single common spiro carbon atom. Examples of spiro ring systems include 3,8-diaza-bicyclo[3.2.1]octane, 2,5-diaza-bicyclo[2.2.1]heptane, 6-azaspiro[3.4]octane, 2-oxa-6-azaspiro[3.4] octane, 2-azaspiro[3.3] heptane, 2-oxa-6-azaspiro[3.3] heptane, 6-oxa-2-azaspiro[3.4] octane, 2,7-diaza-spiro[4.4]nonane, 2-azaspiro[3.5]nonane, 2-oxa-7-azaspiro[3.5]nonane and 2-oxa-6-azaspiro[3.5]nonane.

“Heterocyclyl-Cₘ₋ₙ alkyl” includes a heterocyclyl group covalently attached to a Cₘ₋ₙ alkylene group, both of which are defined herein; and wherein the Heterocyclyl-Cₘ₋ₙ alkyl group is linked to the remainder of the molecule via a carbon atom in the alkylene group. The groups “aryl-Cₘ₋ₙ alkyl”, “heteroaryl-Cₘ₋ₙ alkyl” and “cycloalkyl-Cₘ₋ₙ alkyl” are defined in the same way.

“—Cₘ₋ₙ alkyl substituted by —NRR” and “Cₘ₋ₙ alkyl substituted by —OR” similarly refer to an —NRR″ or —OR″ group covalently attached to a Cₘ₋ₙ alkylene group and wherein the group is linked to the remainder of the molecule via a carbon atom in the alkylene group.

The term “aromatic” when applied to a substituent as a whole includes a single ring or polycyclic ring system with 4n+2 electrons in a conjugated IT system within the ring or ring system where all atoms contributing to the conjugated IT system are in the same plane.

The term “aryl” includes an aromatic hydrocarbon ring system. The ring system has 4n+2 electrons in a conjugated IT system within a ring where all atoms contributing to the conjugated π system are in the same plane. For example, the “aryl” may be phenyl and naphthyl. The aryl system itself may be substituted with other groups.

The term “heteroaryl” includes an aromatic mono- or bicyclic ring incorporating one or more (for example 1-4, particularly 1, 2 or 3) heteroatoms selected from nitrogen, oxygen or sulfur. The ring or ring system has 4n+2 electrons in a conjugated IT system where all atoms contributing to the conjugated IT system are in the same plane.

Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Bicyclic heteroaryl groups can be vicinally fused, i.e. where the rings are linked to each other through two adjacent carbon and/or nitrogen atoms. Each ring may contain up to about four heteroatoms typically selected from nitrogen, sulfur and oxygen. Typically the heteroaryl ring will contain up to 4, for example up to 3 heteroatoms, more usually up to 2, for example a single heteroatom.

In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 1H-pyrazolo[4,3-d]-oxazolyl, 4H-imidazo[4,5-d] thiazolyl, pyrazino[2,3-d]pyridazinyl, imidazo[2,1-b]thiazolyl and imidazo[1,2-b][1,2,4]triazinyl. Examples of heteroaryl groups comprising at least one nitrogen in a ring position include pyrrolyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, indolyl, isoindolyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl and pteridinyl.

“Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Partially aromatic heteroaryl bicyclic ring systems can be vicinally fused, i.e. where the rings are linked to each other through two adjacent carbon and/or nitrogen atoms. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3] dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Where Ring B is heteroaryl and the heteroaryl ring system includes an aromatic ring and a non-aromatic, saturated or partially saturated ring, Ring B is bonded to the group of the formula:

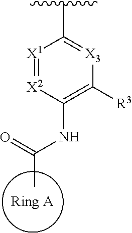

via a ring atom in an aromatic ring of Ring B. By way of illustration, when Ring B is a bicyclic heteroaryl group of the formula:

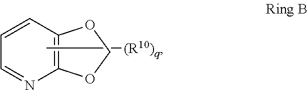

Ring B is bonded to the remainder of the compound of Formula (I) via a carbon atom in the pyridyl ring (i.e. one of the ring carbon atoms marked * below)

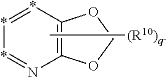

Examples of five-membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six-membered heteroaryl groups include but are not limited to pyridyl, pyrazinyl, pyridazinyl, pyrimidinyl and triazinyl.

Particular examples of bicyclic heteroaryl groups containing a six-membered ring fused to a five-membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl, pyrrolopyridine, and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “oxo,” or “═O” as used herein, means an oxygen that is double bonded to a carbon atom.

The term “optionally substituted” includes either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups, which may be the same or different. For example “one or more optional substituents” may refer to 1 or 2 or 3 substituents (e.g. 1 substituent or 2 substituents).

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without undue effort which substitutions are chemically possible and which are not.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”:

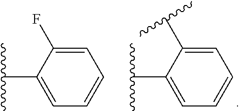

“Meta” substitution is a substitution pattern where two substituents are on carbons one carbon removed from each other, i.e. with a single carbon atom between the substituted carbons. In other words there is a substituent on the second atom away from the atom with another substituent. For example the groups below are meta substituted:

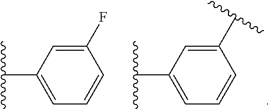

“Para” substitution is a substitution pattern where two substituents are on carbons two carbons removed from each other, i.e. with two carbon atoms between the substituted carbons. In other words there is a substituent on the third atom away from the atom with another substituent. For example the groups below are para substituted:

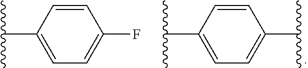

Reference to a —NRR′ group forming a 4 to 6 membered heterocyclyl refers to R and R′ together with the nitrogen atom to which they are attached forming a 4 to 6 membered heterocyclyl group. For example, an —NRR′ such as a —NR^{a1}R^{b1}, —NR^{a2}R^{b2}, —NR^{a3}R^{b3}, —NR^{a4}R^{b4}, —NR^{a8}R^{b8}, —NR^{a9}R^{b9}, —NR^{a10}R^{b10}, —NR⁵R⁶, —NR⁸R⁹ or —NR¹¹R^{a6} group may form:

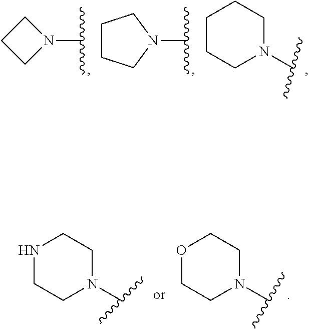

Similarly an —NRR′ group within a substituent may form a carbonyl-linked 4 to 6 membered heterocyclyl, for example a —C(O)NRR′ group may form:

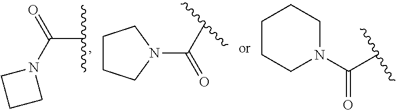

—NRR′ groups within substituents such as —OC(O)NRR′, —SO₂NRR′, or —NRC(O)NRR′, may similarly form a 4 to 6 membered heterocyclyl within such substituents.

A bond terminating in a “ [symbol] ” or “*” represents that the bond is connected to another atom that is not shown in the structure. A bond terminating inside a cyclic structure and not terminating at an atom of the ring structure represents that the bond may be connected to any of the atoms in the ring structure where allowed by valency.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The various functional groups and substituents making up the compounds of the present invention are typically chosen such that the molecular weight of the compound does not exceed 1000. More usually, the molecular weight of the compound will be less than 750, for example less than 700, or less than 650, or less than 600, or less than 550. More preferably, the molecular weight is less than 585 and, for example, is 575 or less.

Suitable or preferred features of any compounds of the present invention may also be suitable features of any other aspect.

The invention contemplates pharmaceutically acceptable salts of the compounds of the invention. These may include the acid addition and base salts of the compounds. These may be acid addition and base salts of the compounds.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of the invention may be prepared by for example, one or more of the following methods:
(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

These methods are typically carried out in solution. The resulting salt may precipitate out and be collected by filtration or may be recovered by evaporation of the solvent. The degree of ionisation in the resulting salt may vary from completely ionised to almost non-ionised.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Where a compound of the invention has two or more stereo centres any combination of (R) and(S) stereoisomers is contemplated. The combination of (R) and(S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%, for example at least 90%, at least 95% or at least 99%.

The compounds of this invention may possess one or more asymmetric centres; such compounds can therefore be produced as individual (R) or(S) stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E and Z isomers). It is to be understood that the present invention encompasses all optical, diastereoisomers and geometric isomers and mixtures thereof

Z/E (e.g. cis/trans) isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high-pressure liquid chromatography (HPLC). Thus, chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and for specific examples, 0 to 5% by volume of an alkylamine e.g. 0.1% diethylamine. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

Compounds and salts described in this specification may be isotopically-labelled (or “radio-labelled”). Accordingly, one or more atoms are replaced by an atom having an atomic mass or mass number different from the atomic mass or mass number typically found in nature. Examples of radionuclides that may be incorporated include ²H (also written as “D” for deuterium), ³H (also written as “T” for tritium), ¹¹C, ¹³C, ¹⁴C, ¹⁵O, ¹⁷O, ¹⁸O, ¹³N, ¹⁵N, ¹⁸F, ³⁶Cl, ¹²³I, ²⁵I, ³²P, ³⁵S and the like. The radionuclide that is used will depend on the specific application of that radio-labelled derivative. For example, for in vitro competition assays, ³H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

Isotopically-labelled compounds can generally be prepared by conventional techniques known to those skilled in the art or by processes analogous to those described using an appropriate isotopically-labelled reagent in place of the non-labelled reagent previously employed.

The selective replacement of hydrogen with deuterium in a compound may modulate the metabolism of the compound, the PK/PD properties of the compound and/or the toxicity of the compound. For example, deuteration may increase the half-life or reduce the clearance of the compound in vivo. Deuteration may also inhibit the formation of toxic metabolites, thereby improving safety and tolerability. It is to be understood that the invention encompasses deuterated derivatives of compounds of formula (I). As used herein, the term deuterated derivative refers to compounds of the invention where in a particular position at least one hydrogen atom is replaced by deuterium. For example, one or more hydrogen atoms in a C₁₋₄-alkyl group may be replaced by deuterium to form a deuterated C₁₋₄-alkyl group. By way of example, if R² is methyl the invention also encompasses —CD₃, —CHD₂ and —CH₂D.

Certain compounds of the invention may exist in solvated as well as unsolvated forms such as, for example, hydrated forms. It is to be understood that the invention encompasses all such solvated forms.

It is also to be understood that certain compounds of the invention may exhibit polymorphism, and that the invention encompasses all such forms.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

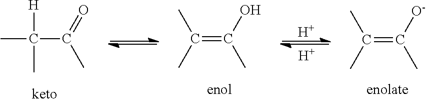

for example

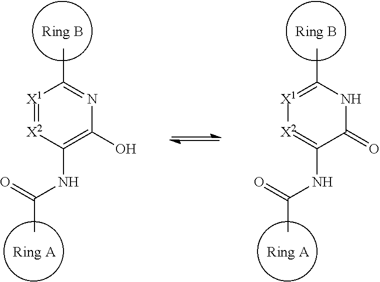

The in vivo effects of a compound of the invention may be exerted in part by one or more metabolites that are formed within the human or animal body after administration of a compound of the invention.

It is further to be understood that a suitable pharmaceutically-acceptable pro-drug of a compound of the formula (I) also forms an aspect of the present invention. Accordingly, the compounds of the invention encompass pro-drug forms of the compounds and the compounds of the invention may be administered in the form of a pro-drug (i.e. a compound that is broken down in the human or animal body to release a compound of the invention). A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo-cleavable ester derivatives that may be formed at a carboxy group or a hydroxy group in a compound of the invention and in vivo-cleavable amide derivatives that may be formed at a carboxy group or an amino group in a compound of the invention.

Accordingly, the present invention includes those compounds of the invention as defined herein when made available by organic synthesis and when made available within the human or animal body by way of cleavage of a pro-drug thereof. Accordingly, the present invention includes those compounds of the formula (I) that are produced by organic synthetic means and also such compounds that are produced in the human or animal body by way of metabolism of a precursor compound, that is a compound of the formula (I) may be a synthetically-produced compound or a metabolically-produced compound.

A suitable pharmaceutically-acceptable pro-drug of a compound of the invention is one that is based on reasonable medical judgement as being suitable for administration to the human or animal body without undesirable pharmacological activities and without undue toxicity.

Various forms of pro-drug have been described, for example in the following documents: —
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the formula I that possesses a carboxy group is, for example, an in vivo-cleavable ester thereof. An in vivo-cleavable ester of a compound of the invention containing a carboxy group is, for example, a pharmaceutically-acceptable ester which is cleaved in the human or animal body to produce the parent acid. Suitable pharmaceutically-acceptable esters for carboxy include C₁₋₆ alkyl esters such as methyl, ethyl and tert-butyl, C₁₋₆ alkoxymethyl esters such as methoxymethyl esters, C₁₋₆ alkanoyloxymethyl esters such as pivaloyloxymethyl esters, 3-phthalidyl esters, C₃₋₈ cycloalkylcarbonyloxy-C₁₋₆ alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆ alkoxycarbonyloxy-C₁₋₆ alkyl esters such as methoxycarbonyloxymethyl and 1-methoxycarbonyloxyethyl esters.

A suitable pharmaceutically-acceptable pro-drug of a compound of the invention that possesses a hydroxy group is, for example, an in vivo-cleavable ester or ether thereof. An in vivo-cleavable ester or ether of a compound of the invention containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include C₁₋₁₀ alkanoyl groups such as acetyl, benzoyl, phenylacetyl and substituted benzoyl and phenylacetyl groups, C₁₋₁₀ alkoxycarbonyl groups such as ethoxycarbonyl, N,N—(C₁₋₆ alkyl)₂ carbamoyl, 2-dialkylaminoacetyl and 2-carboxyacetyl groups. Examples of ring substituents on the phenylacetyl and benzoyl groups include aminomethyl, N-alkylaminomethyl, N, N-dialkylaminomethyl, morpholinomethyl, piperazin-1-ylmethyl and 4-(C₁₋₄ alkyl) piperazin-1-ylmethyl. Suitable pharmaceutically-acceptable ether forming groups for a hydroxy group include α-acyloxyalkyl groups such as acetoxymethyl and pivaloyloxymethyl groups.

A suitable pharmaceutically-acceptable pro-drug of a compound of the invention that possesses a carboxy group is, for example, an in vivo-cleavable amide thereof, for example an amide formed with an amine such as ammonia, a C₁₋₄ alkylamine such as methylamine, a (C₁₋₄ alkyl)₂ amine such as dimethylamine, N-ethyl-N-methylamine or diethylamine, a C₁₋₄ alkoxy-C₂₋₄ alkylamine such as 2-methoxyethylamine, a phenyl-C₁₋₄ alkylamine such as benzylamine and amino acids such as glycine or an ester thereof.

A suitable pharmaceutically-acceptable pro-drug of a compound of the invention that possesses an amino group is, for example, an in vivo-cleavable amide or carbamate derivative thereof. Suitable pharmaceutically-acceptable amides from an amino group include, for example an amide formed with C₁₋₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N, N-dialkylaminomethyl, morpholinomethyl, piperazin-1-ylmethyl and 4-(C₁₋₄ alkyl) piperazin-1-ylmethyl. Suitable pharmaceutically-acceptable carbamates from an amino group include, for example acyloxyalkoxycarbonyl and benzyloxycarbonyl groups.

### Compounds

The following paragraphs are applicable to the compounds of the invention.

In certain embodiments the compound of the formula (I) is a compound of the formula (II), or a pharmaceutically acceptable salt thereof:

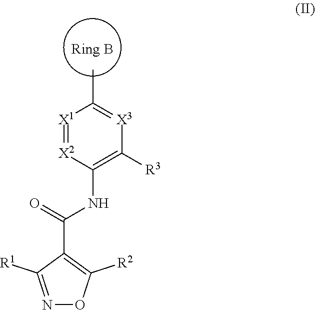

In certain embodiments the compound of the formula (I) is a compound of the formula (III), or a pharmaceutically acceptable salt thereof:

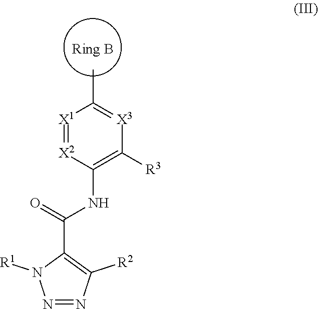

In certain embodiments the compound of the formula (I) is a compound of the formula (IV), or a pharmaceutically acceptable salt thereof:

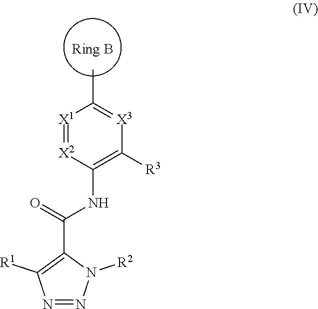

In certain embodiments the compound of the formula (I) is a compound of the formula (V), or a pharmaceutically acceptable salt thereof.

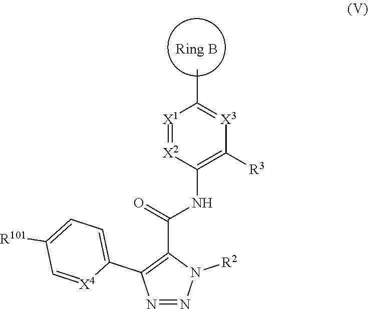

wherein X⁴ is CH or N; and
R¹⁰¹ is selected from H and halo.

In certain embodiments the compound of the formula (I) is a compound of the formula (VI), or a pharmaceutically acceptable salt thereof:

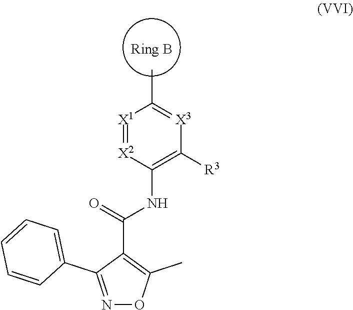

In certain embodiments the compound of the formula (I) is a compound of the formula (VII), or a pharmaceutically acceptable salt thereof.

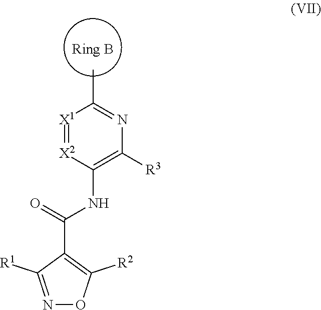

In certain embodiments the compound of the formula (I) is a compound of the formula (VIII), or a pharmaceutically acceptable salt thereof:

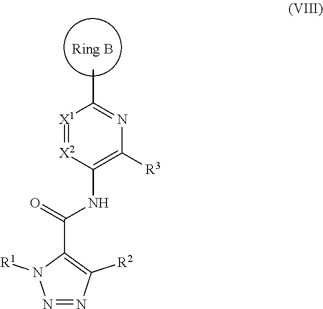

In certain embodiments the compound of the formula (I) is a compound of the formula (IX), or a pharmaceutically acceptable salt thereof:

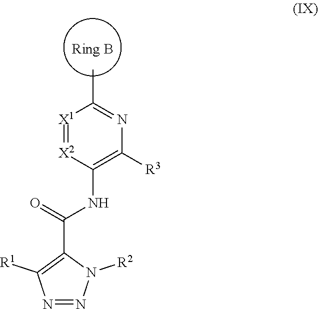

In certain embodiments the compound of the formula (I) is a compound of the formula (X), or a pharmaceutically acceptable salt thereof:

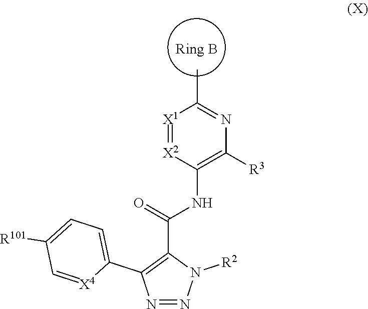

wherein X⁴ is CH or N; and
R¹⁰¹ is selected from H and halo.

In certain embodiments the compound of the formula (I) is a compound of the formula (XI), or a pharmaceutically acceptable salt thereof:

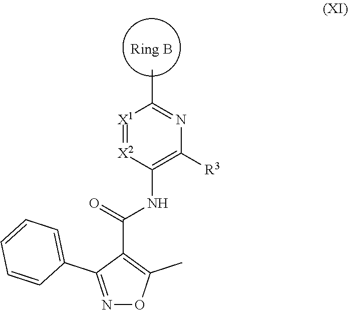

In certain embodiments the compound of the formula (I) is a compound of the formula (XII), or a pharmaceutically acceptable salt thereof.

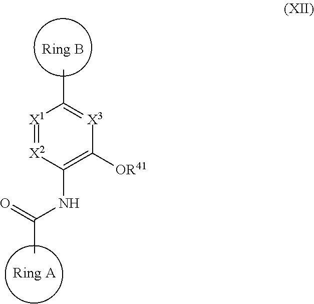

wherein R⁴¹ is C₁₋₃ alkyl (e.g. R⁴¹ is methyl).

In certain embodiments the compound of the formula (I) is a compound of the formula (XIII), or a pharmaceutically acceptable salt thereof:

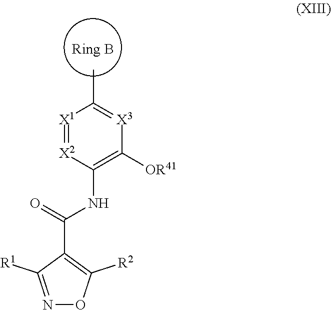

wherein R⁴¹ is C₁₋₃ alkyl (e.g. R⁴¹ is methyl).

In certain embodiments the compound of the formula (I) is a compound of the formula (XIV), or a pharmaceutically acceptable salt thereof.

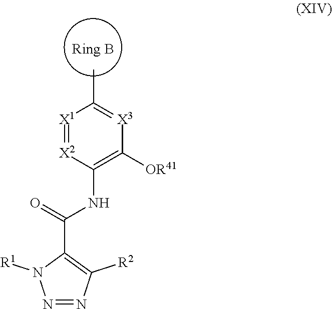

wherein R⁴¹ is C₁₋₃ alkyl (e.g. R⁴¹ is methyl).

In certain embodiments the compound of the formula (I) is a compound of the formula (XV), or a pharmaceutically acceptable salt thereof.

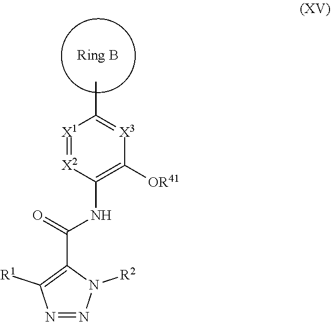

wherein R⁴¹ is C₁₋₃ alkyl (e.g. R⁴¹ is methyl).

In certain embodiments the compound of the formula (I) is a compound of the formula (XVI), or a pharmaceutically acceptable salt thereof:

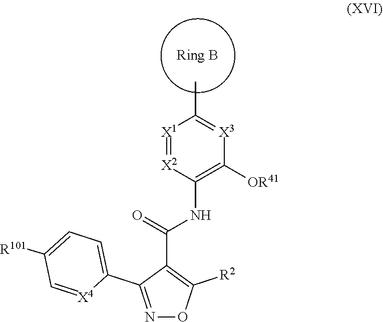

wherein R⁴¹ is C₁₋₃ alkyl (e.g. R⁴¹ is methyl);
X⁴ is CH or N; and
R¹⁰¹ is selected from H and halo.

In certain embodiments the compound of the formula (I) is a compound of the formula (XVII), or a pharmaceutically acceptable salt thereof:

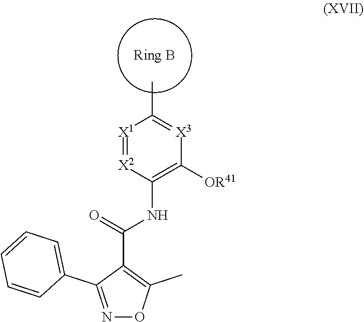

wherein R⁴¹ is C₁₋₃ alkyl (e.g. R⁴¹ is methyl).

In certain embodiments the compound of the formula (I) is a compound of the formula (XVIII), or a pharmaceutically acceptable salt thereof:

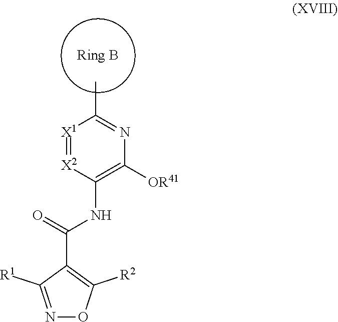

wherein R⁴¹ is C₁₋₃ alkyl (e.g. R⁴¹ is methyl).

In certain embodiments the compound of the formula (I) is a compound of the formula (XIX), or a pharmaceutically acceptable salt thereof:

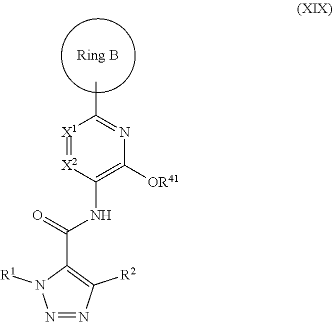

wherein R⁴¹ is C₁₋₃ alkyl (e.g. R⁴¹ is methyl).

In certain embodiments the compound of the formula (I) is a compound of the formula (XX), or a pharmaceutically acceptable salt thereof.

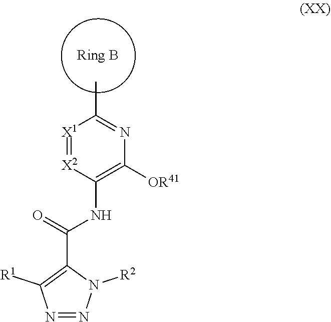

R⁴¹ is Cis alkyl (e.g. R⁴¹ is methyl).

In certain embodiments the compound of the formula (I) is a compound of the formula (XXI), or a pharmaceutically acceptable salt thereof.

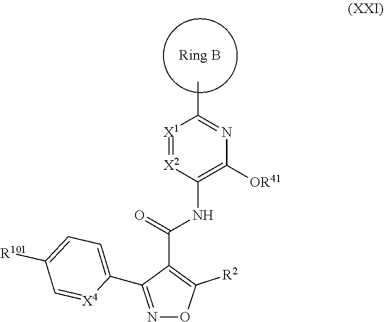

wherein R⁴¹ is C₁₋₃ alkyl (e.g. R⁴¹ is methyl);
wherein X⁴ is CH or N; and
R¹⁰¹ is selected from H and halo.

In certain embodiments the compound of the formula (I) is a compound of the formula (XXII), or a pharmaceutically acceptable salt thereof:

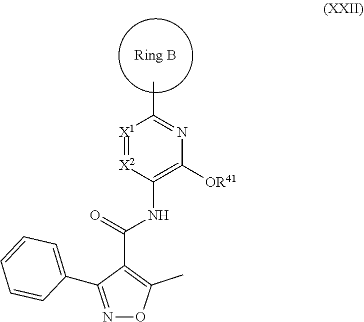

wherein R⁴¹ is C₁₋₃ alkyl (e.g. R⁴¹ is methyl).

In certain embodiments the compound of the formula (I) is a compound of the formula (XXIII), or a pharmaceutically acceptable salt thereof:

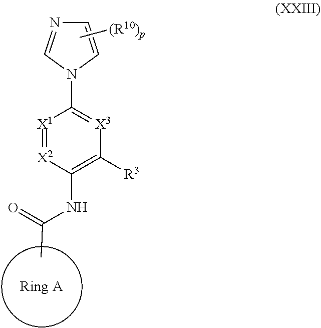

wherein p is 0, 1, 2 or 3.

In certain embodiments the compound of the formula (I) is a compound of the formula (XXIV), or a pharmaceutically acceptable salt thereof.

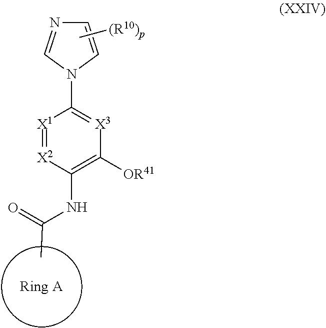

wherein p is 0, 1, 2 or 3; and
R⁴¹ is Cis alkyl (e.g. R⁴¹ is methyl).

In certain embodiments the compound of the formula (I) is a compound of the formula (XXV), or a pharmaceutically acceptable salt thereof.

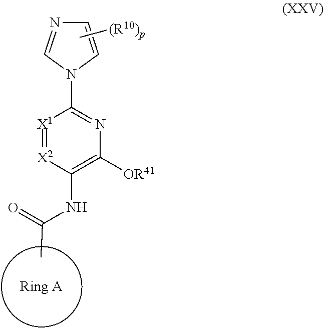

wherein p is 0, 1, 2 or 3; and
R⁴¹ is C₁₋₃ alkyl (e.g. R⁴¹ is methyl).

In certain embodiments the compound of the formula (I) is a compound of the formula (XXVI), or a pharmaceutically acceptable salt thereof:

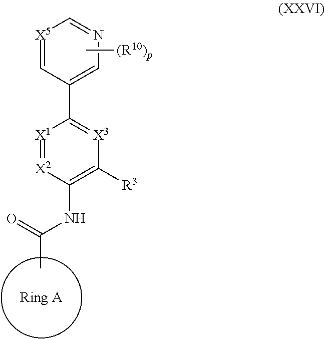

wherein p is 0, 1, 2 or 3; and
X⁵ is CH or N.

In certain embodiments the compound of the formula (I) is a compound of the formula (XXVII), or a pharmaceutically acceptable salt thereof:

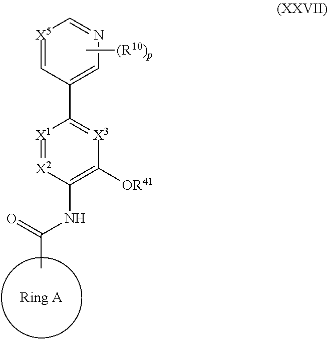

wherein p is 0, 1, 2 or 3;
X⁵ is CH or N; and
R⁴¹ is C₁₋₃ alkyl (e.g. R⁴¹ is methyl).

In certain embodiments the compound of the formula (I) is a compound of the formula (XXVIII), or a pharmaceutically acceptable salt thereof:

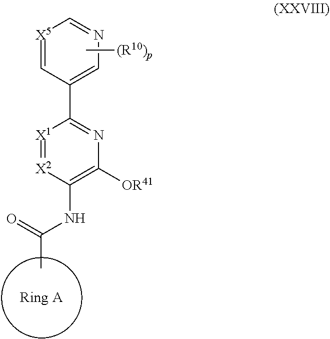

wherein p is 0, 1, 2 or 3;
X⁵ is CH or N; and
R⁴¹ is C₁₋₃ alkyl (e.g. R⁴¹ is methyl).

In certain embodiments the compound of the formula (I) is a compound of the formula (XXIX), or a pharmaceutically acceptable salt thereof:

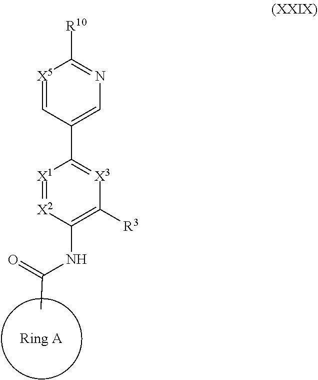

wherein X⁸ is CH or N.

In certain embodiments the compound of the formula (I) is a compound of the formula (XXX), or a pharmaceutically acceptable salt thereof.

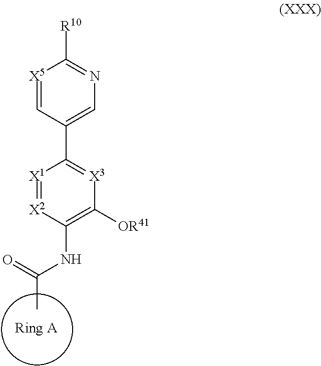

wherein X⁵ is CH or N; and
R⁴¹ is C₁₋₃ alkyl (e.g. R⁴¹ is methyl).

In certain embodiments the compound of the formula (I) is a compound of the formula (XXXI), or a pharmaceutically acceptable salt thereof.

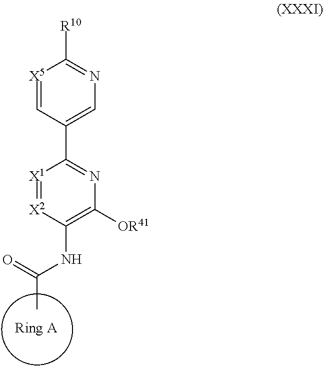

wherein X⁵ is CH or N; and
R⁴¹ is C₁₋₃ alkyl (e.g. R⁴¹ is methyl).

In certain embodiments compounds of the invention include, for example, compounds of formulae (I) to (XXXI), or a pharmaceutically acceptable salt thereof, wherein, unless otherwise stated, each of Ring A, Ring B, R¹, R², R³, R¹⁰, X¹, X², X³, has any of the meanings defined hereinbefore or in any of the following statements in the numbered paragraphs (1) to (203) hereinafter. These statements are independent and interchangeable. In other words, any of the features described in any one of the following statements may (where chemically allowable) be combined with the features described in one or more other statements below. In particular, where a compound is exemplified or illustrated in this specification, any two or more of the statements below which describe a feature of that compound, expressed at any level of generality, may be combined so as to represent subject matter which is contemplated as forming part of the disclosure of this invention in this specification:
1. Ring A is A1 of the structure:

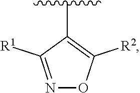

wherein [symbol] indicates the point of attachment as defined in Formula (I).
2. Ring A is A2 of the structure:

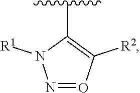

wherein [symbol] indicates the point of attachment as defined in Formula (I).
3. Ring A is A3 and has the structure:

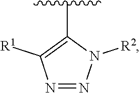

wherein [symbol] indicates the point of attachment as defined in Formula (I).
4. R¹ is phenyl.
5. R¹ is phenyl substituted by one or more substituents selected from: halo, C₁₋₄ alkyl, C₁₋₄ haloalkyl, —OR^{a1}, —SR^{a1} and —NR^{a1}R^{b1}. For example, it may be that R¹ is a phenyl group substituted by one or more substituents selected from halo and —OR^{a1}, wherein R^{a1} may be C₁₋₄ haloalkyl. Thus, it may be that R¹ is a phenyl group substituted with a halo. For example, it may be that R¹ is a phenyl group substituted with —F or —Cl. It may be that R¹ is phenyl substituted by —OR^{a1}. It may be that R¹ is phenyl substituted by —OC₁₋₄ haloalkyl. Thus it may be that R¹ is phenyl substituted by —OCF₃.
6. R¹ is 4-fluorophenyl.
7. R¹ is a 5- or 6-membered heteroaryl group containing at least one ring nitrogen atom, wherein said heteroaryl is optionally substituted with one or more substituents selected from: halo, C₁₋₄ alkyl, C₁₋₄ haloalkyl, —OR^{a1}, —SR^{a1} and —NR^{a1}R^{b1}.
8. R¹ is a 5-membered heteroaryl group containing at least one ring nitrogen, wherein said heteroaryl is optionally substituted with one or more substituents selected from: halo, C₁₋₄ alkyl, C₁₋₄ haloalkyl, —OR^{a1}, —SR^{a1} and —NR^{a1}R^{b1}
9. R¹ is a 6-membered heteroaryl group containing at least one ring nitrogen, wherein said heteroaryl is optionally substituted with one or more substituents selected from: halo, C₁₋₄ alkyl, C₁₋₄ haloalkyl, —OR^{a1}, —SR^{a1} and —NR^{a1}R^{b1}.
10. R¹ is a heteroaryl selected from: isoxazolyl, oxazolyl, isothiazolyl, thiazolyl, pyrazolyl, imidazolyl, pyridyl, pyrazinyl, pyridazinyl or pyrimidinyl, wherein said heteroaryl is optionally substituted with one or more substituents selected from: halo, C₁₋₄ alkyl, C₁₋₄ haloalkyl, —OR^{a1}, —SR^{a1} and —NR^{a1}R^{b1}.
11. R¹ is a heteroaryl selected from: isoxazolyl, oxazolyl, isothiazolyl, thiazolyl, pyrazolyl, imidazolyl, wherein said heteroaryl is optionally substituted with one or more substituents selected from: halo, C₁₋₄ alkyl, C₁₋₄ haloalkyl, —OR^{a1}, —SR^{a1} and —NR^{a1}R^{b1}
12. R¹ is a heteroaryl selected from: pyridyl, pyrazinyl, pyridazinyl or pyrimidinyl, wherein said heteroaryl is optionally substituted with one or more substituents selected from: halo, C₁₋₄ alkyl, C₁₋₄ haloalkyl, —OR^{a1}, —SR^{a1} and —NR^{a1}R^{b1}
13. R¹ is a heteroaryl selected from pyridyl, wherein said heteroaryl is optionally substituted with one or more substituents selected from: halo, C₁₋₄ alkyl, C₁₋₄ haloalkyl, —OR^{a1}, —SR^{a1} and —NR^{a1}R^{b1}.
14. R¹ is a heteroaryl as defined in any one of numbered paragraphs 7 to 13, wherein said heteroaryl is unsubstituted.
15. R¹ is a heteroaryl as defined in any one of numbered paragraphs 7 to 13, wherein said heteroaryl is substituted by one or two substituents selected from: halo, C₁₋₄ alkyl, C₁₋₄ haloalkyl, —OR^{a1}, —SR^{a1} and —NR^{a1}R^{b1}
16. R¹ is a heteroaryl as defined in any one of numbered paragraphs 7 to 13, wherein said heteroaryl is substituted by one or two substituents selected from: halo, C₁₋₄ alkyl, C₁₋₄ haloalkyl and —OR^{a1}.
17. R¹ is a heteroaryl as defined in any one of numbered paragraphs 7 to 13, wherein said heteroaryl is substituted by one or two substituents selected from: halo.
18. R¹ is a heteroaryl as defined in any one of numbered paragraphs 7 to 13, wherein said heteroaryl is substituted by one fluoro substituent.
19. R¹ is selected from:

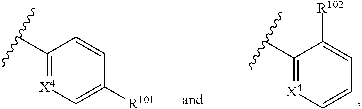

wherein:

X⁴ is CH or N;
R¹⁰¹ is H or halo; and
R¹⁰² is —OC₁₋₄ haloalkyl.
20. R¹ has the structure:

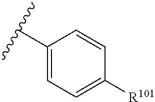

wherein R¹⁰¹ is H or halo. Thus it may be that R¹⁰¹ is F or Cl.
21. R¹ is selected from:

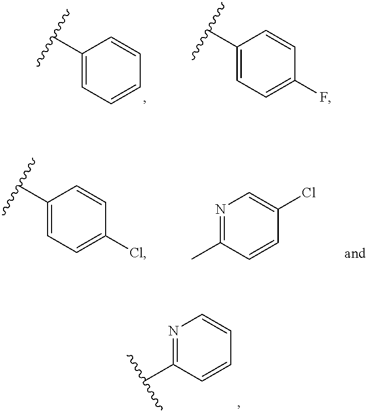

wherein [symbol] indicates the point of attachment to Ring A.
22. Ring A is A1 and R¹ is selected from the group consisting of:

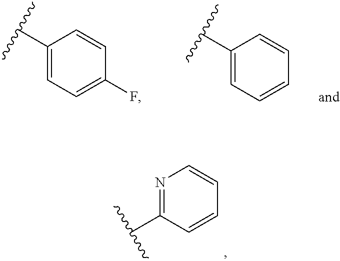 [SMILES: *c1ccc(F)cc1 ; *c1ccccc1 ; *c1ccccn1]

wherein [symbol] indicates the point of attachment to Ring A.
23. Ring A is A1 and R¹ is selected from the group consisting of:

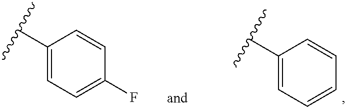 [SMILES: *c1ccc(F)cc1 ; *c1ccccc1]

wherein [symbol] indicates the point of attachment to Ring A.
24. Ring A is A2 and R¹ is unsubstituted phenyl.
25. Ring A is A3 and R¹ is selected from the group consisting of:

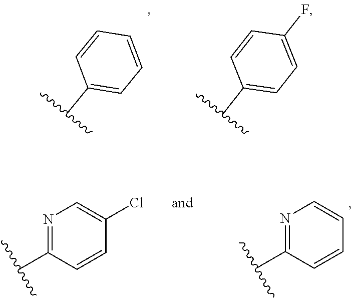 [SMILES: *c1ccccc1 ; *c1ccc(F)cc1 ; *c1ccc(Cl)cn1 ; *c1ccccn1]

wherein [symbol] indicates the point of attachment to Ring A.
26. R² is selected from: halo, C₁₋₄ alkyl and C₁₋₄ haloalkyl, —OR^{a2}, —SR^{a2} and —NR^{a2}R^{b2}, wherein the C₁₋₄ alkyl is optionally substituted by one or more substituents selected from: halo, —OR^{a3}, —SR^{a3} and —NR^{a3}R^{b3}
27. R² is selected from: H, halo, C₁₋₄ alkyl and C₁₋₄ haloalkyl, —OR^{a2}, —SR^{a2} and —NR²²R^{b2}.
28. R² is selected from: halo, C₁₋₄ alkyl and C₁₋₄ haloalkyl, —OR^{a2}, —SR^{a2} and —NR^{a2}R^{b2}.
29. R² is H.
30. R² is C₁₋₄ alkyl.
31. R² is C₁₋₃ alkyl
32. R² is methyl.
33. R² is methyl and Ring A is A1.
34. R² is methyl and Ring A is A2.
35. R² is methyl and Ring A is A3.
36. R² is methyl and R¹ is as defined in any one of numbered paragraphs 4 to 25.
37. Ring A is selected from the group consisting of:

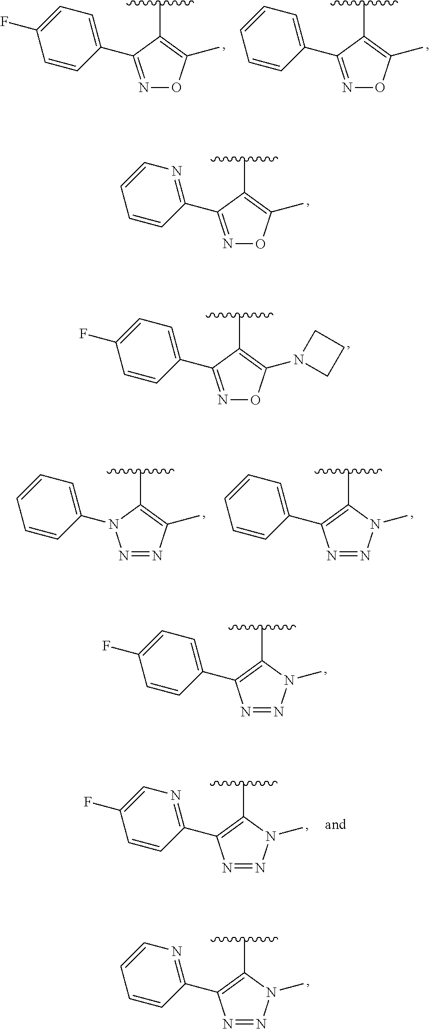

wherein [symbol] indicates the point of attachment as defined in Formula (I).
38. Ring A is selected from:

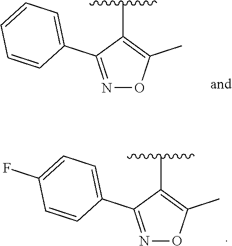 [SMILES: *c1c(-c2ccccc2)noc1C ; *c1c(-c2ccc(F)cc2)noc1C]

39. R³ is selected from: C₁₋₄ alkyl, C₁₋₄ haloalkyl, C₃₋₆ cycloalkyl, C₃₋₆ cycloalkyl-C₁₋₃ alkyl-, —OR⁴, —NR⁵R⁶, —SR⁵, 4- to 7-membered heterocyclyl containing 1 or more ring oxygen atoms, and 4- to 7-membered heterocyclyl-C₁₋₃ alkyl-containing 1 or more ring oxygen atoms, wherein any of said C₁₋₄ alkyl, C₃₋₆ cycloalkyl or C₃₋₆ cycloalkyl-C₁₋₃ alkyl- is optionally substituted by one or more substituents selected from: halo, —OR^{a4}, —SR^{a4} and —NR^{a4}R^{b4}
40. R³ is selected from: C₁₋₄ alkyl, C₁₋₄ haloalkyl, C₃₋₈ cycloalkyl, C₃₋₈ cycloalkyl-C₁₋₃ alkyl-, —OR⁴, —SR⁵, 4- to 7-membered heterocyclyl containing 1 or more ring oxygen atoms, and 4- to 7-membered heterocyclyl-C₁₋₃ alkyl-containing 1 or more ring oxygen atoms, wherein any of said C₁₋₄ alkyl, C₃₋₆ cycloalkyl or C₃₋₆ cycloalkyl-C₁₋₃ alkyl- is optionally substituted by one or more substituents selected from: halo, —OR^{a4}, and —SR^{a4}
41. R³ is selected from: C₁₋₄ haloalkyl, C₃₋₆ cycloalkyl, C₃₋₆ cycloalkyl-C₁₋₃ alkyl-, —OR⁴, —SR⁵, 4- to 7-membered heterocyclyl containing 1 or more ring oxygen atoms, and 4- to 7-membered heterocyclyl-C₁₋₃ alkyl-containing 1 or more ring oxygen atoms, wherein any of said C₃₋₆ cycloalkyl or C₃₋₆ cycloalkyl-C₁₋₃ alkyl- is optionally substituted by one or more substituents selected from: halo, —OR^{a4}, and —SR^{a4}.
42. R³ is selected from: 4- to 7-membered heterocyclyl containing 1 or more ring oxygen atoms, 4- to 7-membered heterocyclyl-C₁₋₃ alkyl-containing 1 or more ring oxygen atoms, and —OR⁴.
43. R³ is selected from: 4- to 7-membered heterocyclyl containing 1 or more ring oxygen atoms, 4- to 7-membered heterocyclyl-C₁₋₃ alkyl-containing 1 or more ring oxygen atoms.
44. R³ is a 4- to 7-membered heterocyclyl containing 1, or 2 ring oxygen atoms. Preferably, it may be that R³ is a 4- to 7-membered heterocyclyl comprising 1 ring oxygen atom. Thus, it may be that R³ is selected from oxetanyl, tetrahydrofuranyl, and pyranyl.
45. R³ is a 4- to 7-membered heterocyclyl-C₁₋₃ alkyl containing 1, or 2 ring oxygen atoms. Preferably, it may be that R³ is a 4- to 7-membered heterocyclyl-C₁₋₃ alkyl-containing 1 ring oxygen atom. Thus, it may be that R³ is selected from oxetanyl-C₁₋₃ alkyl-, tetrahydrofuranyl-C₁₋₃ alkyl-, and pyranyl-C₁₋₃ alkyl-.
46. R³ is selected from: C₁₋₄ alkyl, C₁₋₄ haloalkyl, C₃₋₆ cycloalkyl, C₃₋₆ cycloalkyl-C₁₋₃ alkyl-, —OR⁴, —NR⁵R⁶ and —SR⁵, wherein any of said C₁₋₄ alkyl, C₃₋₆ cycloalkyl or C₃₋₆ cycloalkyl-C₁-3 alkyl- is optionally substituted by one or more substituents selected from: halo, —OR^{a4}, —SR^{a4} and —NR^{a4}R^{b4}
47. R³ is selected from: —OR⁴ and —NR⁵R⁶.
48. R³ is selected from: —OR⁴ and —NR⁵R⁶, wherein R⁴ is selected from: C₃₋₆ cycloalkyl, C₁₋₄ alkyl and C₂₋₄ alkyl substituted by —NR^{a4}R^{b4}; R^{a4} and R^{b4} are independently selected from: H and C₁₋₄ alkyl; and R⁵ and R⁶ are independently selected from: H and C₁₋₄ alkyl.
49. R³ is —NR⁵R⁶.
50. R³ is —NH₂.
51. It may be that R³ is not —NR⁵R⁶. Thus, it may be that R³ is not —NH₂.
52. It may be that R³ is not C₁₋₄ alkyl. It may be that R³ is not C₁₋₄ alkyl or —NR⁵R⁶.
53. R³ is —OR⁴.
54. R³ is —OR⁴ wherein R⁴ is selected from C₁₋₄ alkyl, C₁₋₄ haloalkyl, C₃₋₆ cycloalkyl, C₃₋₆ cycloalkyl-C₁₋₃ alkyl-, 4- to 7-membered heterocyclyl containing 1 or 2 ring oxygen atoms, and 4- to 7-membered heterocyclyl-C₁₋₃ alkyl-containing 1 or 2 ring oxygen atoms, wherein said C₁₋₄ alkyl, C₃₋₆ cycloalkyl or C₃₋₆ cycloalkyl-C₁₋₃ alkyl- in R⁴ is optionally substituted by one or more substituents selected from: halo, —OR^{a4}, —SR^{a4} and —NR^{a4}R^{b4}
55. R³ is —OR⁴ wherein R⁴ is 4- to 7-membered heterocyclyl containing 1 or 2 ring oxygen atoms, or 4- to 7-membered heterocyclyl-C₁₋₃ alkyl-containing 1 or 2 ring oxygen atoms.
56. R³ is —OR⁴ wherein R⁴ is 4- to 6-membered heterocyclyl, containing 1, or 2 ring oxygen atoms. Preferably, it may be that R³ is —OR⁴ wherein R⁴ is 4- to 6-membered heterocyclyl, containing 1 ring oxygen atom. Thus, it may be that R³ is —OR⁴ wherein R⁴ is selected from oxetanyl, tetrahydrofuranyl, and pyranyl.
57. R³ is —OR⁴ wherein R⁴ is 4- to 6-membered heterocyclyl-C₁₋₃ alkyl-, containing 1, or 2 ring oxygen atoms. It may be that R³ is —OR⁴ wherein R⁴ is 4- to 6-membered heterocyclyl-C₁₋₃ alkyl-, comprising 1 ring oxygen atom. Thus, it may be that R³ is —OR⁴ wherein R⁴ is selected from oxetanyl-C₁₋₃ alkyl-, tetrahydrofuranyl-C₁₋₃ alkyl-, and pyranyl-C₁₋₃ alkyl-.
58. R³ is:

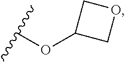 [SMILES: *OC1COC1]

wherein [symbol] indicates the point of attachment to the remainder of the compound of Formula (I).
59. R³ is —OR⁴ wherein R⁴ is selected from C₁₋₄ alkyl, C₁₋₄ haloalkyl, C₃₋₆ cycloalkyl and C₃₋₆ cycloalkyl-C₁₋₃ alkyl- wherein said C₁₋₄ alkyl, C₃₋₆ cycloalkyl or C₃₋₆ cycloalkyl-C₁₋₃ alkyl- in R⁴ is optionally substituted by one or more substituents selected from: halo, —OR^{a4}, —SR^{a4} and —NR^{a4}R^{b4}
60. R³ is —OR⁴ wherein R⁴ is selected from: C₁₋₄ alkyl, C₁₋₄ haloalkyl and C₃₋₆ cycloalkyl.
61. R³ is —OR⁴ wherein R⁴ is selected from: C₁₋₄ alkyl and C₁₋₄ haloalkyl.
62. R³ is —OR⁴ wherein R⁴ is C₁₋₄ alkyl.
63. R³ is —OR⁴ wherein R⁴ is C₂₋₄ alkyl, substituted by one or more substituents selected from: —OR^{a4}, —SR^{a4} and —NR^{a4}R^{b4}
64. R³ is —OR⁴ wherein R⁴ is C₂₋₄ alkyl, substituted by —OR^{a4}, wherein R^{a4} is selected from H and C₁₋₄ alkyl. It may be that R⁴ is C₂₋₄ alkyl, substituted by —OR^{a4}, wherein R^{a4} is H. Thus, it may be that R³ is —O(CH₂)₂OH. It may be that R⁴ is C₂₋₄ alkyl, substituted by —OR^{a4}, wherein R^{a4} is C₁₋₄ alkyl. Thus, it may be that R³ is —O(CH₂)₂OCH₃.
65. R³ is —OR⁴ wherein R⁴ is C₂₋₄ alkyl substituted by —NR^{a4}R^{b4}, wherein R^{a4} and R^{b4} are independently selected from H and C₁₋₄ alkyl. It may be that R⁴ is C₂₋₄ alkyl, substituted by —NR^{a4}R^{b4}, wherein R^{a4} is H and R^{b4} is C₁₋₄ alkyl. Thus, it may be that R³ is —O(CH₂)₂NHCH₃.
66. R³ is —OR⁴ wherein R⁴ is C₃₋₆ cycloalkyl.
67. R³ is selected from: cyclopropoxy, cyclobutoxy and cyclopentoxy.
68. R³ is cyclobutoxy.
69. R³ is selected from: —OC₁₋₃ alkyl and —OC₃₋₄ cycloalkyl.
70. R³ is selected from: methoxy and —OCH(CH₃)₂.
71. R³ is selected from methoxy and —O-cyclobutyl.
72. R³ is methoxy.
73. R³ is —OCH(CH₃)₂.
74. R³ is —O-cyclobutyl.
75. R³ is —OH.
76. R³ is —NR⁵R⁶. It may be that R⁵ and R⁶ are independently selected from H and C₁₋₄ alkyl. It may be that R⁵ is H and R⁶ is C₁₋₄ alkyl. It may be that R⁵ is H and R⁶ is methyl. It may be that R⁵ and R⁶ are C₁₋₄ alkyl. It may be that R⁵ is methyl and R⁶ is methyl.
77. R³ is selected from: —OR⁴ and —NR⁵R⁶,
   wherein R⁴ is selected from: C₃₋₆ cycloalkyl, C₁₋₄ alkyl and C₂₋₄ alkyl substituted by —NR^{a4}R^{b4}
   R^{a4} and R^{b4} are independently selected from H and C₁₋₄ alkyl; and
   R⁵ and R⁶ are independently selected from H and C₁₋₄ alkyl.
78. R³ is: —OR⁴,
   wherein R⁴ is selected from: C₃₋₆ cycloalkyl, C₁₋₄ alkyl, and C₂₋₄ alkyl optionally substituted by —OR^{a4}. It may be that R³ is —OR⁴ and R⁴ is selected from: C₃₋₆ cycloalkyl, C₁₋₄ alkyl.
79. R³ is selected from: methoxy,

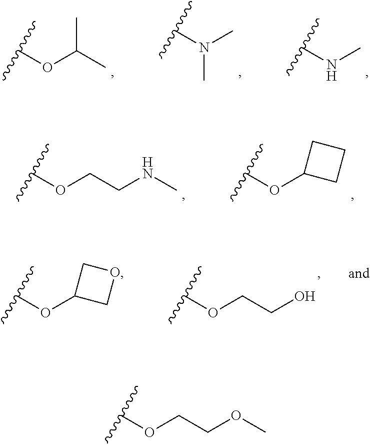

wherein [symbol] indicates the point of attachment to the remainder of the compound of Formula (I).
80. R³ is selected from: methoxy,

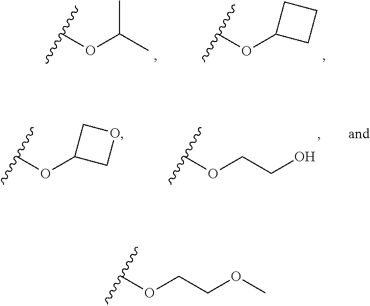

wherein [symbol] indicates the point of attachment to the remainder of the compound of Formula (I).
81. R³ is selected from: methoxy,

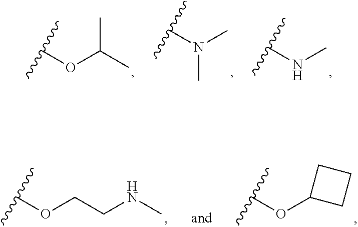

wherein [symbol] indicates the point of attachment to the remainder of the compound of Formula (I). It may be that R³ is selected from: methoxy,

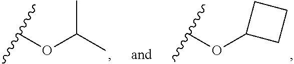

82. X¹ is N.
83. X¹ is CR⁷.
84. X¹ is CH.
85. X¹ is CR⁷, wherein R⁷ is C₁₋₄ alkyl, optionally substituted by one or more substituents selected from: halo, —CN, —OR^{a5}, —S(O)ₓR^{a5} (wherein x is 0, 1, or 2) and —NR^{a5}R^{b5}.
86. X₁ is CR⁷, wherein R⁷ is C₁₋₃ alkyl, substituted by —NR²⁵R^{b5}.
87. X₁ is CR⁷, wherein R⁷ is C₁₋₃ alkyl, substituted by —NR^{a5}R^{b5}, wherein R^{a5} and R^{b5} are selected from: H and C₁₋₃ alkyl.
88. X¹ is CR⁷, wherein R⁷ is —CH₂N(CH₃)₂.
89. X² is N.
90. X² is CR⁷.
91. X² is CH.
92. X³ is N.
93. X³ is CR⁷.
94. X³ is CH.
95. X³ is N, and X¹ and X² are CR⁷.
96. X³ is N, X′ is CR⁷ and X² is CH.
97. X³ is N, X′ is CH and X² is CR⁷.
98. X³ is N, and X¹ and X² are CH.
99. X² and X³ are N and X¹ is CR⁷.
100. X² and X³ are N and X¹ is CR⁷, wherein R⁷ is —CH₂N(CH₃)₂.
101. X² and X³ are N and X¹ is CH.
102. X¹ and X² are N and X³ is CR⁷.
103. X¹ and X² are N and X³ is CH.
104. X¹ is N, and X² and X³ are CR⁷.
105. X¹ is N, X² is CH and X³ is CR⁷.
106. X¹ is N, X² is CR⁷ and X³ is CH.
107. X¹ is N, and X² and X³ are CH.
108. X² is N, and X¹ and X³ are CR⁷.
109. X² is N, X¹ is CR⁷ and X³ is CH.
110. X² is N, X¹ is CH and X³ is CR⁷.
111. X² is N, and X¹ and X³ are CH.
112. X¹, X² and X³ are CH.
113. R⁷ is at each occurrence independently selected from: H, C₁₋₄ haloalkyl and C₁₋₄ alkyl, optionally substituted by one or more substituents selected from: —CN, —OR^{a5}, —S(O)ₓR^{a5} (wherein x is 0, 1, or 2) and —NR^{a5}R^{b5}
114. R⁷ is at each occurrence independently selected from: H, C₁₋₃ haloalkyl and C₁₋₃ alkyl, optionally substituted by one substituent selected from: —OR^{a5}, —S(O)₂R^{a5} and —NR^{a5}R^{b5}
115. R⁷ is at each occurrence independently selected from: H, C₁₋₄ haloalkyl and C₁₋₄ alkyl.
116. R⁷ is H.
117. Ring B is selected from: phenyl and a monocyclic or bicyclic 5- to 12-membered heteroaryl, each of which is optionally substituted with one or more R¹⁰.
118. Ring B is selected from: phenyl and a monocyclic or bicyclic 5- to 11-membered heteroaryl, each of which is optionally substituted with one or more R¹⁰.
119. Ring B is selected from: phenyl, a monocyclic 5- or 6-membered heteroaryl, each of which is optionally substituted with one or more R¹⁰.
120. Ring B is selected from: a 5- or 6-membered heteroaryl, each of which is optionally substituted with one or more R¹⁰.
121. Ring B is selected from: phenyl, a 5- or 6-membered heteroaryl and a 9- or 10-membered bicyclic heteroaryl, each of which is optionally substituted with one or more R¹⁰. It maybe that Ring B is selected from: a monocyclic 5- or 6-membered heteroaryl and a 9- or 10-membered bicyclic heteroaryl, each of which is optionally substituted with one or more R¹⁰.
122. Ring B is selected from: phenyl and a bicyclic 8- to 10-membered heteroaryl optionally substituted with one or more R¹⁰.
123. Ring B is selected from: a monocyclic or bicyclic 5- to 11-membered heteroaryl optionally substituted with one or more R¹⁰
124. Ring B is selected from: a bicyclic 8-, 9- or 10-membered heteroaryl optionally substituted with one or more R¹⁰.
125. Ring B is selected from: a bicyclic 9- or 10-membered heteroaryl optionally substituted with one or more R¹⁰.
126. Ring B is selected from: a bicyclic 9-membered heteroaryl optionally substituted with one or more R¹⁰.
127. Ring B is selected from: a bicyclic 10-membered heteroaryl optionally substituted with one or more R¹⁰.
128. Ring B is selected from: a 5-membered heteroaryl optionally substituted with one or more R¹⁰.
129. Ring B is selected from: a 6-membered heteroaryl optionally substituted with one or more R¹⁰.
130. Ring B is as defined in any one of numbered paragraphs 117 to 129, wherein said heteroaryl contains from 1 to 4 ring heteroatoms selected from O, S and N.
131. Ring B is as defined in any one of numbered paragraphs 117 to 129, wherein said heteroaryl contains 1 ring nitrogen atom and optionally 1 to 3 ring heteroatoms selected from O, S and N.
132. Ring B is as defined in any one of numbered paragraphs 117 to 129, wherein said heteroaryl contains 1 ring nitrogen atom and optionally 1 or 2 ring heteroatoms selected from O, S and N.
133. Ring B is as defined in any one of numbered paragraphs 117 to 129, wherein said heteroaryl contains from 1 to 4 ring nitrogen atoms. Thus is may be that the heteroaryl contains from 1 to 3 ring nitrogen atoms. For example, the heteroaryl contains 1 or 2 ring nitrogen atoms.
134. Ring B is as defined in any one of numbered paragraphs 117 to 133, wherein when Ring B is heteroaryl, said heteroaryl is bonded to the remainder of the compound of Formula (I) via a ring carbon atom in an aromatic ring in Ring B.
135. Ring B is as defined in any one of numbered paragraphs 117 to 133, wherein when Ring B is heteroaryl, said heteroaryl is bonded to the remainder of the compound of Formula (I) via a ring nitrogen atom in an aromatic ring in Ring B.
136. Ring B is as defined in any one of numbered paragraphs 117 to 135, wherein Ring B is optionally substituted by 1 or 2 R¹⁰. Thus it may be that Ring B is substituted by 1 R¹⁰. It may be that Ring B is substituted by 1 R¹⁰.
137. Ring B is selected from:

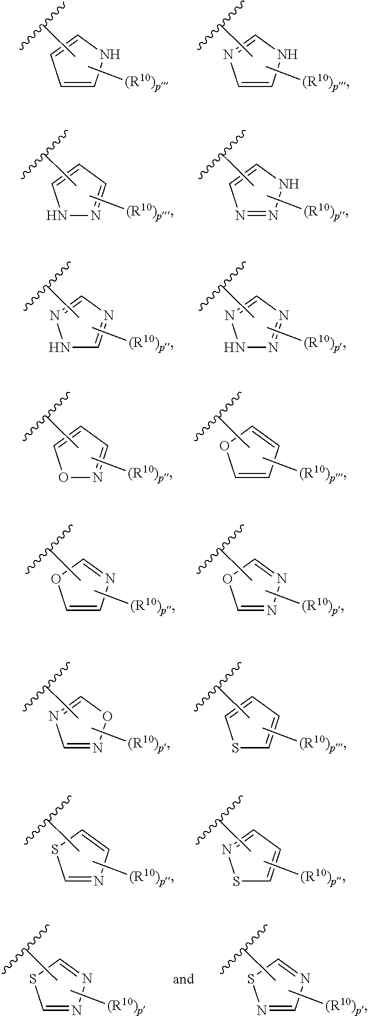

wherein p′ is 0 or 1;
p″ is 0, 1 or 2;
p′″ is 0, 1, 2 or 3; and
[symbol] indicates the point of attachment to the remainder of the compound of Formula (I). It may be that p′, p″ and p′″ are 0 or 1. It may be that p′, p″ and p″ are 0.
138. Ring B is selected from:

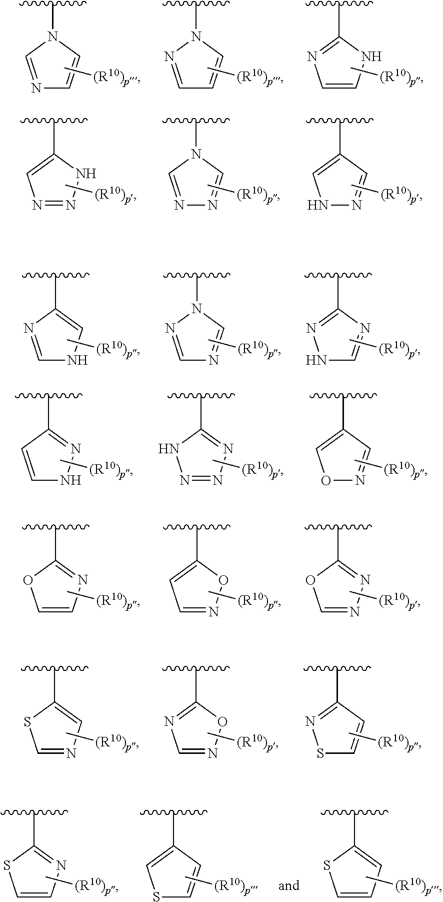

wherein p′ is 0 or 1;
p″ is 0, 1 or 2;
p″ is 0, 1, 2 or 3; and
[symbol] indicates the point of attachment to the remainder of the compound of Formula (I). It may be that p′, p″ and p″ are 0 or 1. It may be that p′, p″ and p″ are 0.
139. Ring B is selected from:

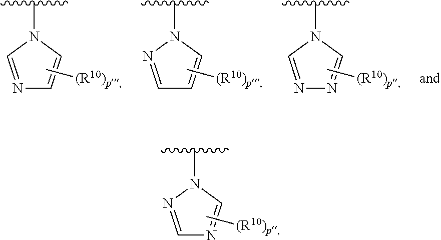

wherein p″ is 0, 1 or 2;
p″ is 0, 1, 2 or 3; and
[symbol] indicates the point of attachment to the remainder of the compound of Formula (I). It may be that p″ and p″ are 0 or 1. It may be that p″ and p″ are 0.
140. Ring B is:

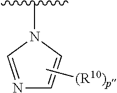

wherein p″ is 0, 1 or 2; and [symbol] indicates the point of attachment to the remainder of the compound of Formula (I).
141. Ring B is:

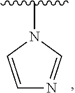 [SMILES: *n1ccnc1]

wherein [symbol] indicates the point of attachment to the remainder of the compound of Formula (I).
142. Ring B is selected from:

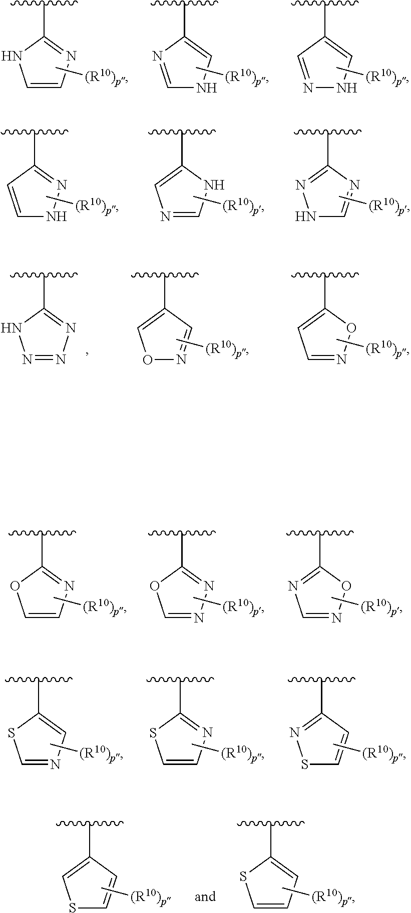

wherein p′ is 0 or 1; and
p′ is 0, 1 or 2; and
[symbol] indicates the point of attachment to the remainder of the compound of Formula (I). It may be that p′ and p″ are 0 or 1. It may be that p′ and p″ are 0.
143. Ring B is a 6-membered heteroaryl comprising at least one nitrogen in the ring, optionally substituted with one or more (for example 1, 2, or 3) R¹⁰. It may be that Ring B has 1, 2, or 3 ring nitrogen atoms. For example, Ring B has 1 or 2 ring nitrogen atoms.
144. Ring B has the structure:

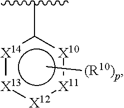

wherein:
X¹⁰, X¹¹, X¹², X¹³ and X¹⁴ are independently selected from CH and N, wherein at least one of X¹⁰, X¹, X¹², X¹³ and X¹⁴ is N;
p is 0, 1, 2, 3 or 4 (where chemically possible); and
[symbol] indicates the point of attachment to the remainder of the compound of Formula (I).
Suitably no more than two of X¹⁰, X¹¹, X¹², X¹³ and X¹⁴ are N.
145. Ring B is selected from:

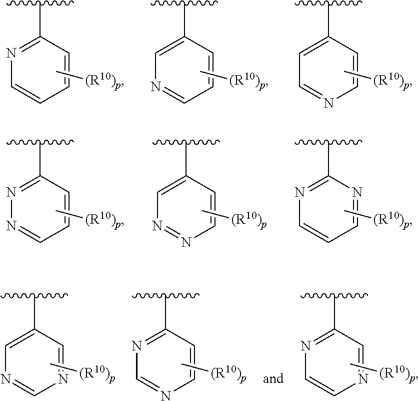

wherein p is 0, 1, 2 or 3, and wherein [symbol] indicates the point of attachment to the remainder of the compound of Formula (I). Typically, p is 0 or 1. For example p is 0. For example p is 1.
146. Ring B has the structure:

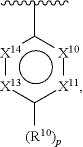

wherein:
X¹⁰, X¹¹, X¹³ and X¹⁴ are independently selected from CH and N, wherein at least one of X¹⁰, X¹¹, X¹³ and X¹⁴ is N;
p is 0, 1, 2 or 3 (where chemically possible); and
[symbol] indicates the point of attachment to the remainder of the compound of Formula (I).
Suitably no more than two of X¹⁰, X¹¹, X¹², X¹³ and X¹⁴ are N
147. Ring B is selected from:

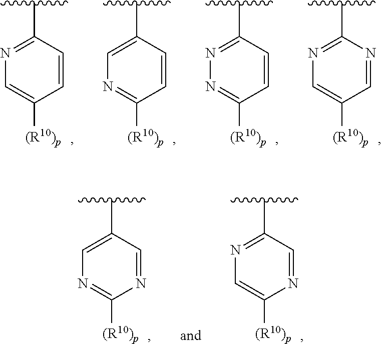

wherein p is 0 or 1; and [symbol] indicates the point of attachment to the remainder of the compound of Formula (I). Optionally p is 1, thus Ring B may be selected from:

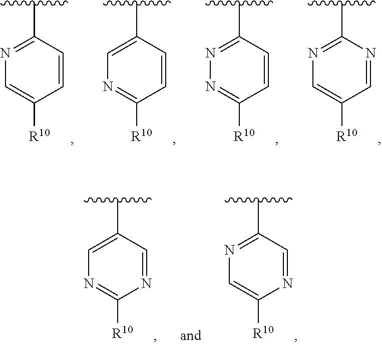

148. Ring B is selected from:

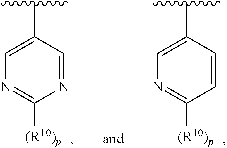

wherein p is 0 or 1; and [symbol] indicates the point of attachment to the remainder of the compound of Formula (I). Optionally p is 1, thus Ring B may be selected from:

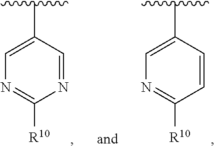

149. Ring B is selected from a 5- or 6-membered heteroaryl ring, wherein said 5-membered heteroaryl is as defined in numbered paragraph (137) and said 6-membered heteroaryl is as defined in numbered paragraph (145).
150. Ring B is selected from a 5- or 6-membered heteroaryl ring, wherein said 5-membered heteroaryl is as defined in numbered paragraph (138) and said 6-membered heteroaryl is as defined in numbered paragraph (145).
151. Ring B is a fused bicyclic heteroaryl selected from: a phenyl or a 5- or 6-membered heteroaryl ring fused to a ring selected from: phenyl, 5- or 6-membered heteroaryl, saturated 4- to 7-membered heterocyclyl, partially saturated 4- to 7-membered heterocyclyl and C₃₋₇ cycloalkyl, wherein Ring B is optionally substituted with one or more R¹⁰ groups, and wherein at least one ring atom in Ring B is N and wherein Ring B is bonded to the remainder of the compound of Formula (I) by a ring atom in an aromatic ring of Ring B.
152. Ring B is a fused bicyclic heteroaryl selected from: a phenyl ring fused to a ring selected from: 5- or 6-membered heteroaryl, 4- to 7-membered saturated heterocyclyl and 4- to 7-membered partially saturated heterocyclyl, wherein Ring B is optionally substituted with one or more R¹⁰ groups, and wherein at least one ring atom in Ring B is N. It may be that Ring B is a bicyclic heteroaryl selected from: a phenyl ring fused to a ring selected from 5- or 6-membered heteroaryl, a 5- or 6-membered saturated heterocyclyl and a 5- or 6-membered partially saturated heterocyclyl, wherein Ring B is optionally substituted with one or more R¹⁰ groups, and wherein at least one ring atom in Ring B is N. In each case above the Ring B group is bonded to the remainder of the compound of Formula (I) by a ring carbon atom in the phenyl ring of the bicyclic Ring B group.
153. Ring B has the structure:

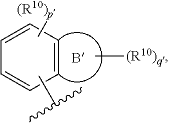

wherein:
Ring B′ is selected from: a 5- or 6-membered heteroaryl ring, 5- or 6-membered saturated heterocyclyl ring and a 5- or 6-membered partially saturated heterocyclyl ring;
p′ and q′ are independently selected from 0, 1, 2 or 3 (where chemically possible);
wherein at least one ring atom in Ring B′ is N; and
wherein Ring B is bonded to the remainder of the compound of Formula (I) by a ring atom in the phenyl ring of Ring B, represented by [symbol] . Suitably p′+q′ is ≤4, for example, p′+q′ is 0, 1 or 2.
154. Ring B is selected from:

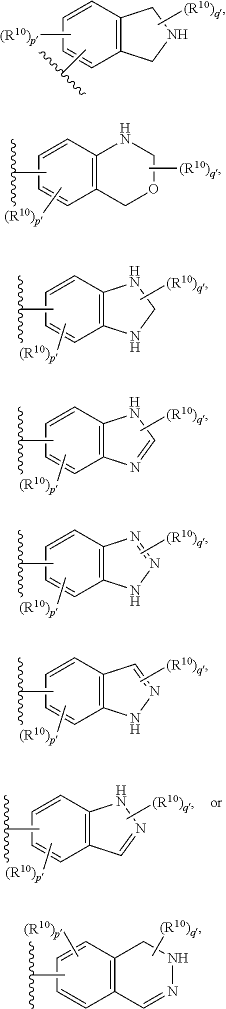

wherein [symbol] indicates the point of attachment to a carbon atom in the phenyl ring of Ring B to the remainder of the compound of Formula (I); and
p′ and q′ are independently selected from 0, 1, 2 or 3 (where chemically possible). Suitably p′+q′ is ≤4. For example p′+q′ is 0, 1 or 2.
155. Ring B is selected from:

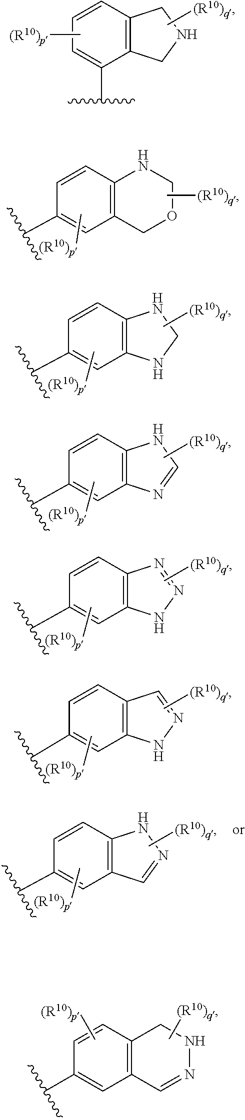

wherein [symbol] indicates the point of attachment or Ring B to the remainder of the compound of Formula (I); and
p′ and q′ are independently selected from 0, 1, 2 or 3 (where chemically possible). Suitably p′+q′ is ≤4. For example p′+q′ is 0, 1 or 2.
156. Ring B is a fused bicyclic heteroaryl selected from: a 6-membered heteroaryl ring fused to a ring selected from a 4- to 7-membered saturated heterocyclyl, a 4- to 7-membered partially saturated heterocyclyl and C₃₋₆ cycloalkyl, wherein the bicyclic group is optionally substituted with one or more R¹⁰ groups, and wherein at least one ring atom in Ring B is N. It may be that Ring B is a fused bicyclic heteroaryl selected from a 6-membered heteroaryl ring fused to a ring selected from: a 5- or 6-membered saturated heterocyclyl, a partially saturated 5- or 6-membered heterocyclyl and a C₅₋₆ cycloalkyl, wherein the bicyclic group is optionally substituted with one or more R¹⁰ groups, and wherein at least one ring atom in Ring B is N. In each case above, Ring B is bonded to the remainder of the compound of Formula (I) by a ring atom in the 6-membered heteroaryl ring of in Ring B.
157. Ring B has the structure:

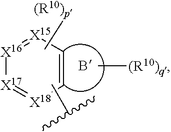

wherein:
Ring B′ is a 5- or 6-membered saturated heterocyclyl, a partially saturated 5- or 6-membered heterocyclyl, or C₃₋₆ cycloalkyl;
X¹⁵, X¹⁶, X¹⁷ and X¹⁸ are independently selected from CH and N, provided that at least one but not more than two of X¹⁵, X¹⁶, X¹⁷ and X¹⁸ is N, and wherein Ring B is bonded to the remainder of the compound of Formula (I) via a ring carbon represented by one of X¹⁵, X¹⁶, X¹⁷ and X¹⁸;
p′ is 0, 1 or 2; q′ is 0, 1, 2 or 3 (where chemically possible). Suitably p′+q′ is ≤4, for example 0, 1 or 2.
158. Ring B is selected from:

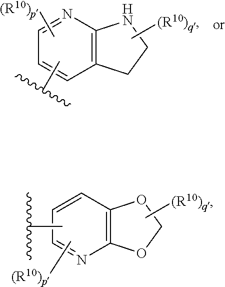

wherein;
p′ is 0, 1 or 2 (where chemically possible); and q′ is 0, 1, 2 or 3 (where chemically possible); and [symbol] indicates the point of attachment from a ring carbon in the pyridyl ring of Ring B to the remainder of the compound in Formula (I). Suitably p′+q′ is ≤4, for example 0, 1 or 2.
159. Ring B is selected from:

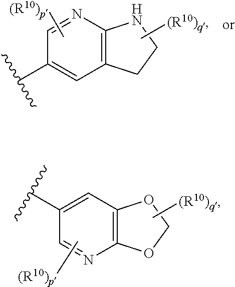

wherein
p′ is 0, 1 or 2 (where chemically possible); and q′ is 0, 1, 2 or 3 (where chemically possible); and [symbol] indicates the point of attachment to the remainder of the compound in Formula (I). Suitably p′+q′ is ≤4, for example 0, 1 or 2.
160. Ring B is a fused bicyclic heteroaryl selected from a 5-membered heteroaryl ring fused to a ring selected from: 4- to 7-membered saturated heterocyclyl, partially saturated 4- to 7-membered heterocyclyl and C₃₋₆ cycloalkyl, wherein Ring B is optionally substituted with one or more R¹⁰ groups, and wherein at least one ring atom in Ring B is N. It may be that Ring B is a fused bicyclic heteroaryl selected from: a 5-membered heteroaryl ring fused to a ring selected from 5- or 6-membered heterocyclyl, partially saturated 5- or 6-membered heterocyclyl and C₅₋₆ cycloalkyl, wherein Ring B is optionally substituted with one or more R¹⁰ groups, and wherein at least one ring atom in Ring B is N. In each of the fused bicyclic heteroaryl Ring B groups above, Ring B is bonded to the remainder of the compound of Formula (I) by a ring atom in the 5-membered heteroaryl ring of Ring B group.
161. Ring B has the structure:

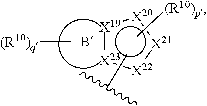

wherein:
Ring B contains at least one ring nitrogen;
Ring B′ is selected from: a 5- or 6-membered saturated heterocyclyl ring, a 5- or 6-membered partially saturated heterocyclyl ring, and C₅₋₆ cycloalkyl;
the ring formed by X¹⁹, X²⁰, X²¹, X²² and X²³ is a 5-membered heteroaryl ring;
X¹⁹ and X²³ are independently selected from C and N;
X²⁰, X²¹ and X²² are independently selected from CH, N, NH, O and S, provided that no more than one of X²⁰, X²¹ and X²² is O, S or NH;
Ring B is bonded to the remainder of the compound in Formula (I) by a ring carbon or nitrogen atom represented by one of X²⁰, X²¹ and X²²
p′ is 0, 1 or 2; and
q′ is 0, 1, 2, 3 or 4.
Suitably p′+q′ is ≤4, for example, p′+q′ is 0, 1 or 2.
162. Ring B is selected from:

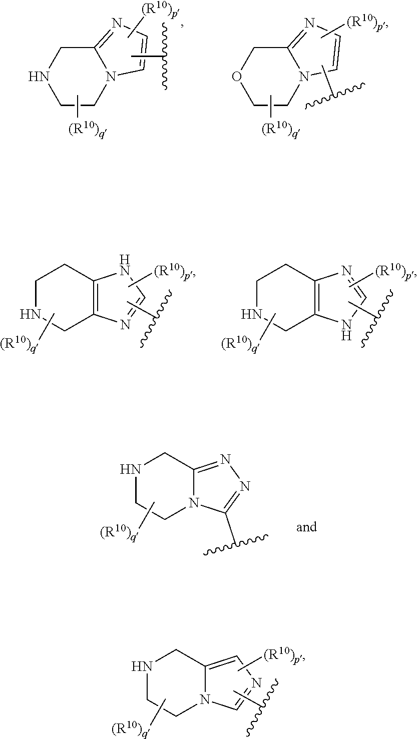

wherein [symbol] indicates the point of Ring B to the remainder of the compound of Formula (I); and wherein Ring B is bonded to the remainder of the compound by a ring carbon or nitrogen atom in the 5-membered heteroaryl ring in Ring B;
p′ is 0 or 1; and
q′ is 0, 1, 2, 3 or 4.
Suitably p′+q′ is ≤4, for example, p′+q′ is 0, 1 or 2.
163. Ring B is selected from:

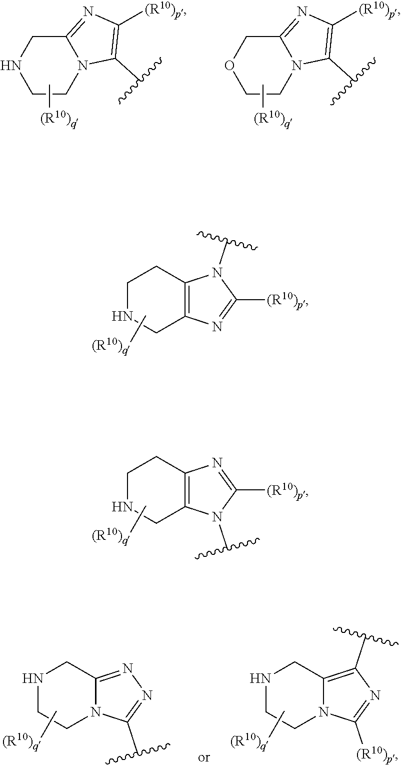

wherein [symbol] indicates the point of Ring B to the remainder of the compound of Formula (I);
p′ is 0 or 1; and
q′ is 0, 1, 2, 3 or 4.
   Suitably p′+q′ is ≤4, for example, p′+q′ is 0, 1 or 2.
164. Ring B is a fused bicyclic heteroaryl selected from: a 5- or 6-membered heteroaryl fused to another 5- or 6-membered heteroaryl, wherein Ring B is optionally substituted with one or more R¹⁰ groups, and wherein at least one ring atom in Ring B is N. It may be that Ring B is a fused bicyclic heteroaryl selected from: a 5-membered heteroaryl fused to a 6-membered heteroaryl, wherein Ring B is optionally substituted with one or more R¹⁰ groups, and wherein at least one ring atom in Ring B is N. It may be that Ring B is a fused bicyclic heteroaryl selected from: a 6-membered heteroaryl fused to another 6-membered heteroaryl, wherein Ring B is optionally substituted with one or more R¹⁰ groups, and wherein at least one ring atom in Ring B is N.
165. Ring B is a fused bicyclic heteroaryl selected from: a 5-membered heteroaryl fused to a 6-membered heteroaryl, wherein ring B is bonded to the remainder of the compound in Formula (I) via a ring carbon or ring nitrogen atom in the 5-membered heteroaryl and wherein Ring B contains at least one ring nitrogen atom (e.g. 1, 2, 3 or 4 ring nitrogen atoms) and optionally one or two ring atoms selected from O and S; and wherein Ring B is optionally substituted with one or more R¹⁰ groups.
166. Ring B is a fused bicyclic heteroaryl selected from: a 5-membered heteroaryl fused to a 6-membered heteroaryl, wherein Ring B is bonded to the remainder of the compound in Formula (I) via a ring carbon or ring nitrogen atom in the 5-membered heteroaryl;
   wherein said 5-membered heteroaryl is selected from pyrrole, oxazole, isoxazole, thiazole, isothiazole, pyrazole, imidazole, 1,2,3-triazole and 1,2,4-triazole; and
   said 6-membered heteroaryl is selected from pyridine, pyrimidine, pyridazine and pyrazine; and
   wherein Ring B is optionally substituted with one or more (for example 1, 2, 3 or 4) R¹⁰ groups.
167. Ring B is a fused bicyclic heteroaryl selected from a 6-membered heteroaryl fused to a 5-membered heteroaryl, wherein Ring B is bonded to the remainder of the compound in Formula (I) via a ring carbon or ring nitrogen atom in the 6-membered heteroaryl and wherein Ring B contains at least one ring nitrogen atom (e.g. 1, 2, 3 or 4 ring nitrogen atoms) and optionally one or two ring atoms selected from O and S; and wherein Ring B is optionally substituted with one or more (for example 1, 2, 3 or 4) R¹⁰ groups.
168. Ring B is a fused bicyclic heteroaryl selected from: a 6-membered heteroaryl fused to a 5-membered heteroaryl, wherein ring B is bonded to the remainder of the compound in Formula (I) via a ring atom in the 6-membered heteroaryl;
   wherein said 5-membered heteroaryl is selected from: pyrrole, oxazole, isoxazole, thiazole, isothiazole, pyrazole, imidazole, 1,2,3-triazole and 1,2,4-triazole; and
   said 6-membered heteroaryl is selected from pyridine, pyrimidine, pyridazine and pyrazine; and
   wherein Ring B is optionally substituted with one or more (for example 1, 2, 3 or 4) R¹⁰ groups.
169. Ring B is a fused bicyclic heteroaryl selected from a 6-membered heteroaryl fused to another 6-membered heteroaryl, wherein Ring B contains at least one ring nitrogen atom (e.g. 1, 2, 3 or 4 ring nitrogen atoms) and optionally one or two ring atoms selected from O and S; and wherein Ring B is optionally substituted with one or more (for example 1, 2, 3 or 4) R¹⁰ groups.
170. Ring B is a fused bicyclic heteroaryl selected from a 6-membered heteroaryl fused to another 6-membered heteroaryl, wherein each of said 6-membered heteroaryl is independently selected from: pyridine, pyrimidine, pyridazine and pyrazine; and
   wherein Ring B is optionally substituted with one or more (for example 1, 2, 3 or 4) R¹⁰ groups.
171. Ring B is a fused bicyclic heteroaryl selected from a 5-membered heteroaryl fused to a phenyl ring, wherein said 5 membered heteroaryl group has 1, 2 or 3 ring heteroatoms selected from O, S and N; and wherein Ring B is optionally substituted with one or more (for example 1, 2, 3 or 4) R¹⁰ groups; wherein Ring B is optionally substituted with one or more (for example 1, 2, 3 or 4) R¹⁰ groups. It may be that Ring B is bonded to the remainder of the compound of Formula (I) by a ring atom in the 5-membered heteroaryl ring. It may be that Ring B is bonded to the remainder of the compound of Formula (I) by a ring atom in the phenyl ring.
172. Ring B is a fused bicyclic heteroaryl group selected from a 5-membered heteroaryl fused to a phenyl ring, wherein said 5 membered heteroaryl group is selected from: pyrrole, oxazole, isoxazole, thiazole, isothiazole, pyrazole, imidazole, 1,2,3-triazole and 1,2,4-triazole; and
   wherein Ring B is optionally substituted with one or more (for example 1, 2, 3 or 4) R¹⁰ groups.
   It may be that Ring B is bonded to the remainder of the compound of Formula (I) by a ring atom in the 5-membered heteroaryl ring. It may be that Ring B is bonded to the remainder of the compound of Formula (I) by a ring atom in the phenyl ring.
173. Ring B is a fused bicyclic heteroaryl selected from: a 6-membered heteroaryl fused to a phenyl ring, wherein said 6 membered heteroaryl has 1, 2 or 3 ring heteroatoms selected from O, S and N; and wherein Ring B is optionally substituted with one or more (for example 1, 2, 3 or 4) R¹⁰ groups. It may be that Ring B is bonded to the remainder of the compound of Formula (I) by a ring atom in the 6-membered heteroaryl ring. It may be that Ring B is bonded to the remainder of the compound of Formula (I) by a ring atom in the phenyl ring.
174. Ring B is a fused bicyclic group selected from a 6-membered heteroaryl fused to a phenyl ring, wherein said 6 membered heteroaryl group is selected from: pyridine, pyrimidine, pyridazine and pyrazine; and
   wherein Ring B is optionally substituted with one or more (for example 1, 2, 3 or 4) R¹⁰ groups.
   It may be that Ring B is bonded to the remainder of the compound of Formula (I) by a ring atom in the 6-membered heteroaryl ring. It may be that Ring B is bonded to the remainder of the compound of Formula (I) by a ring atom in the phenyl ring.
175. Ring B is selected from:

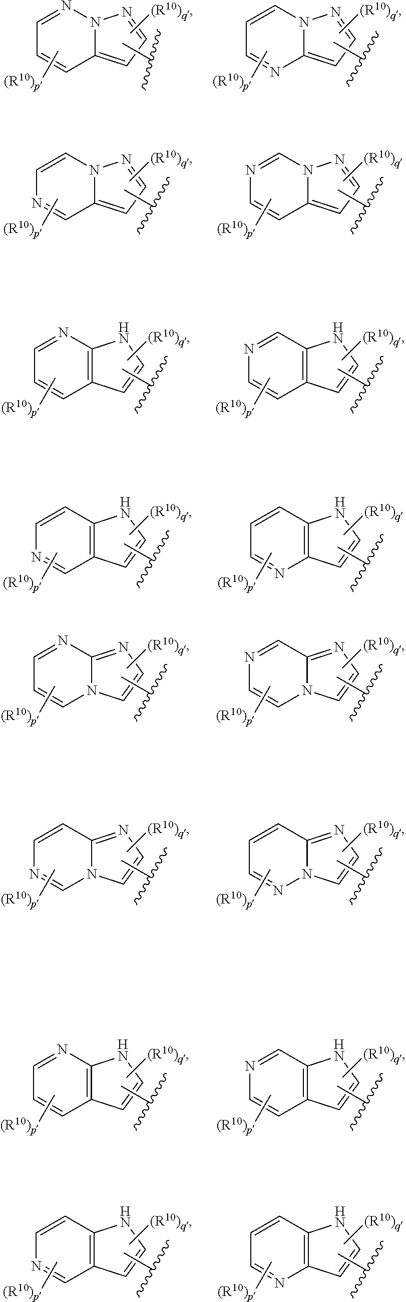

and,
wherein
p′ is 0, 1, 2 or 3;
q′ is 0, 1 or 2 (where chemically possible); and
[symbol] indicates the point of attachment from a ring atom in the 5-membered heteroaryl ring of Ring B to the remainder of the compound in Formula (I).
176. Ring B is selected from:

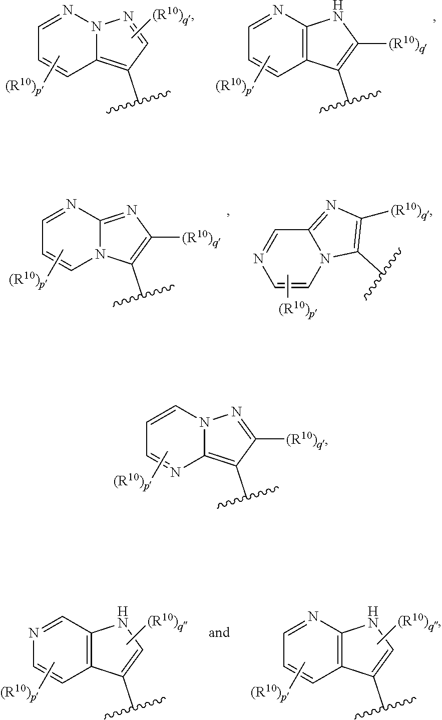

wherein
p′ is 0, 1, 2 or 3;
q′ is 0, 1 or 2 (where chemically possible); and
[symbol] indicates the point of attachment to the remainder of the compound in Formula (I).
177. Ring B is selected from:

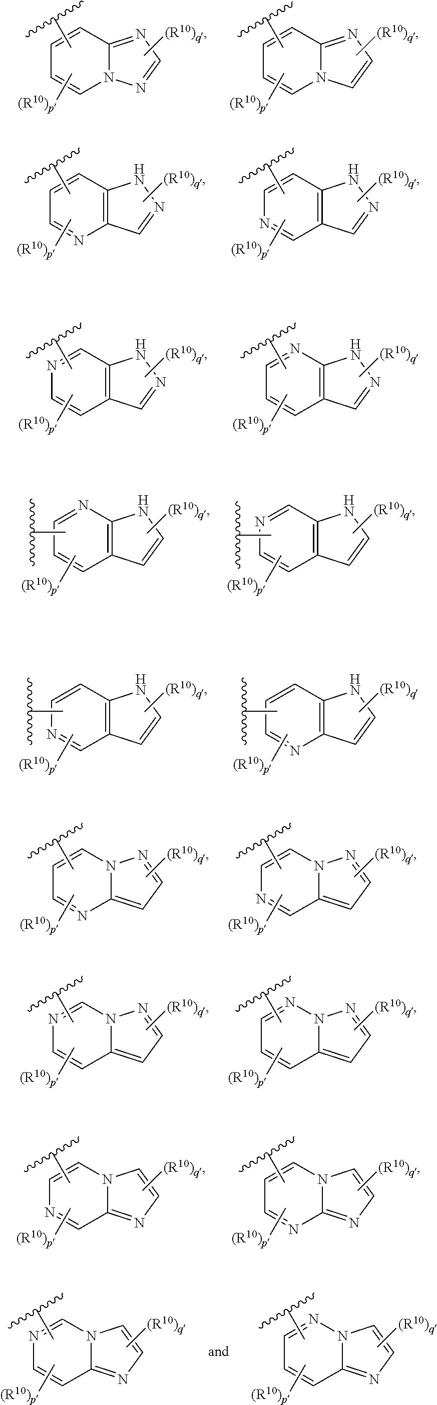

p′ is 0, 1, 2 or 3;
q′ is 0, 1 or 2 (where chemically possible); and
[symbol] indicates the point of attachment from a ring carbon atom in the 6-membered heteroaryl ring of Ring B to the remainder of the compound in Formula (I)
178. Ring B is selected from:

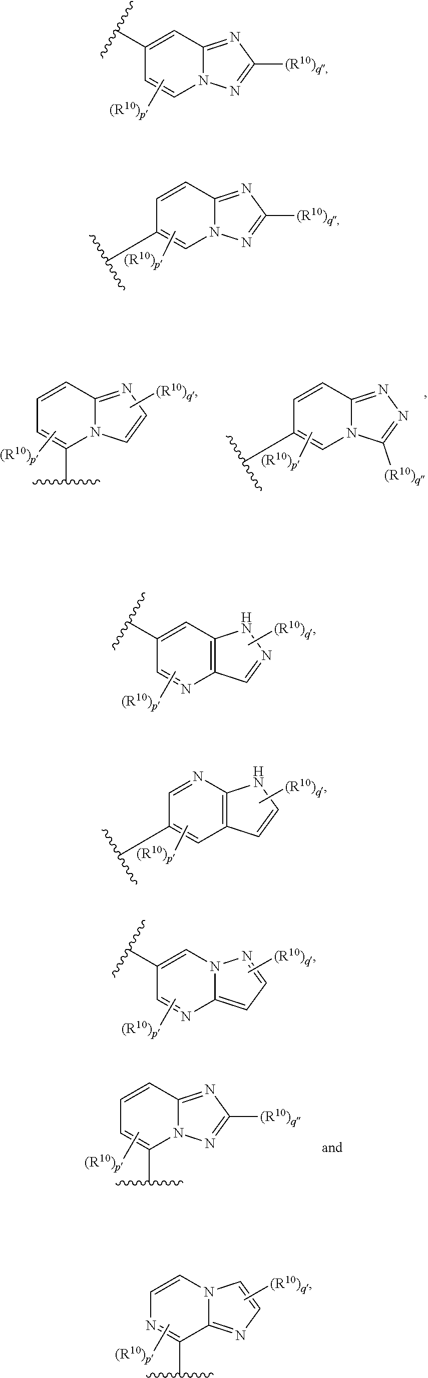

p′ is 0, 1, 2 or 3;
q′ is 0, 1 or 2 (where chemically possible);
q″ is 0 or 1; and
[symbol] indicates the point of attachment to the remainder of the compound in Formula (I).
179. Ring B is C₆-10 aryl, optionally substituted with one or more (for example 1, 2, 3 or 4) R¹⁰.
180. Ring B is phenyl optionally substituted by one or more (for example 1, 2, 3 or 4) R¹⁰.
Ring B is phenyl substituted by one R¹⁰. Thus, it may be that Ring B is selected from:

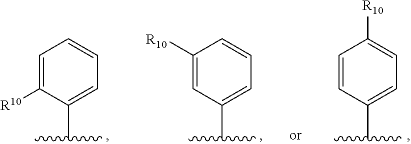

wherein
[symbol] indicates the point of attachment to the remainder of the compound in Formula (I). Preferably, it may be that Ring B is

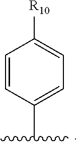

181. Ring B is unsubstituted. Thus R¹⁰ may be absent.
182. Ring B is as defined in any one of numbered paragraphs 117 to 180, wherein R¹⁰ is absent.
183. Ring B is substituted by one or more R¹⁰. For example Ring B is substituted by 1 or 2 R¹⁰. Thus Ring B may be substituted with one R¹⁰.
184. Ring B is as defined in any one on numbered paragraphs 117 to 180, and Ring B is substituted by 1 or 2 R¹⁰.
185. R¹⁰ at each occurrence is independently selected from: halo, —CN, ═O, C₁₋₆ alkyl, C₁-6 haloalkyl, Q¹-L¹-, —OR¹¹, —S(O)₂R¹¹ (wherein x is 0, 1, or 2), —NR¹¹R^{a6}, —C(O)R¹¹, —OC(O)R¹¹, —C(O)OR¹¹, —NR^{a6}C(O)R¹¹, —NR^{a6}C(O)OR¹¹, —C(O)NR¹¹R^{a6}, —OC(O)NR¹¹R^{a6}, —NR^{a6}SO₂R¹¹, —SO₂NR¹¹R^{a6}, wherein said C₁₋₆ alkyl, C₂₋₆ alkenyl and C₂₋₆ alkynyl is optionally substituted by 1 or more R¹². It may be that Ring B is as defined in any one of numbered paragraphs 117 to 184.
186. R¹⁰ at each occurrence is independently selected from: halo, —CN, ═O, C₁₋₆ alkyl, —C₁₋₆ alkyl-OR^{a8}, —C₁₋₆ alkyl-NR^{a8}R^{b8}, —C₁₋₆ alkyl-O(CO) R^{a8}, —C₁₋₆ alkyl-(CO) OR^{a8}, —C₁₋₆ alkyl-SO₂R^{a8}, —OH, —OC₁₋₆ alkyl, —OC₁₋₆ haloalkyl, —OC₂₋₄ alkyl-OR^{a8}, —OC₂₋₄ alkyl-NR^{a8}R^{b8}, —NH₂, —NR^{a6}C₁₋₆ alkyl, —NR^{a6}C₂₋₄ alkyl-OR^{a8}, —NR^{a6}C₂₋₄ alkyl-NR^{a8}R^{b8}, C₁₋₆ haloalkyl, —SC₁₋₆ alkyl, —SC₁₋₆ haloalkyl, —S(O)₂C₁₋₆ alkyl, —S(O)₂C₁₋₆ haloalkyl-C(O)C₁₋₆ alkyl, —C(O)C₁₋₆ haloalkyl, —C(O)C₁₋₆ alkyl-OR^{a8}, —C(O)C₁₋₆ alkyl-NR^{a8}R^{b8}, —OC(O)C₁₋₆ alkyl, —COOH, —C(O)OC₁₋₆ alkyl, —NR^{a6}C(O)C₁₋₆ alkyl, —NR^{a6}C(O)C₁₋₆ alkyl-OR^{a8}, —NR^{a6}C(O)C₁₋₆ alkyl-NR^{a8}R^{b8}, —NR^{a6}C(O)OR^{a8}, —C(O)NR^{a6}C₁₋₆ alkyl, —C(O)NR^{a6}C₂₋₆ alkyl-OR^{a8}, —C(O)NR^{a6}C₂₋₆ alkyl-NR^{a8}R^{b8}—OC(O)NR^{a8}R^{a6}, —NR^{a6}SO₂R^{a8}, —SO₂NR^{a8}R^{a6} and Q¹⁰¹-L¹⁰¹-;
   wherein Q¹⁰¹ is selected from C₃₋₆ cycloalkyl, 4 to 6 membered heterocyclyl, 5- or 6-membered heteroaryl,
   wherein said C₃₋₆ cycloalkyl and 4 to 6 membered heterocyclyl is optionally substituted by one or more (e.g. 1 or 2) substituents selected from: halo, ═O, C₁₋₄ alkyl, —OR^{a8}, —NR^{a8}R^{b8}, —C(O)R^{a8} and —S(O)₂R^{a8},
   wherein said 5- or 6-membered heteroaryl is optionally substituted by one or more (e.g. 1 or 2) substituents selected from: halo, C₁₋₄ alkyl, —OR^{a10}, —NR^{a10}R^{b10}, —C(O)R^{a10} and —S(O)₂R^{a1}; and
   L¹⁰¹ is a bond or is selected from: C₁₋₃ alkylene, —O— and —NR^{a7}—.
   It may be that Ring B is as defined in any one of numbered paragraphs 117 to 184.
187. R¹⁰ at each occurrence is independently selected from: halo, —CN, C₁₋₄ alkyl, C₁₋₄ haloalkyl, —C₁₋₄ alkyl-NR^{a8}R^{b8}, —OH, —OC₁₋₄ alkyl, —OC₁₋₄ haloalkyl, —OC₂₋₄ alkyl-NR^{a8}R^{b8}, —NH₂, —NR^{a6}C₁₋₄ alkyl, —NR^{a6}C₂₋₄ alkyl-OR^{a8}, —NR^{a6}C₂₋₄ alkyl-NR^{a8}R^{b8}, —C(O)C₁₋₄ alkyl, —C(O)C₁₋₄ haloalkyl, —C(O)C₁₋₄ alkyl-NR^{a8}R^{b8}, —COOH, —C(O)OC₁₋₄ alkyl, —C(O)NR^{a6}C₁₋₄ alkyl, —C(O)NR^{a6}C₂₋₄ alkyl-OR^{a8}, —C(O)NR^{a6}C₂₋₄ alkyl-NR^{a8}R^{b8} and Q¹⁰²-L¹⁰²-;
   wherein Q¹⁰² is selected from 4 to 6 membered heterocyclyl and 5- or 6-membered heteroaryl,
   wherein said 4 to 6 membered heterocyclyl is selected from: azetidinyl, pyrrolidinyl, piperidinyl, piperazinyl and morpholinyl, each of which is optionally substituted by one or more (e.g. 1 or 2) substituents selected from: halo, ═O, C₁₋₄ alkyl, —OR^{a8}, —NR^{a8}R^{b8} and —C(O)R^{a8},
   wherein said 5- or 6-membered heteroaryl is selected from: pyrrolyl, imidazolyl, oxazolyl, isoxazolyl, thiazolyl, isothiazolyl, pyrazolyl, oxadiazolyl, triazolyl, pyridyl, pyrimidyl, pyrazinyl and pyridazinyl, each of which is optionally substituted by one or more (e.g. 1 or 2) substituents selected from: halo, C₁₋₄ alkyl, —OR^{a10} and —NR^{a10}R^{b10}.
   L¹⁰² is a bond or is selected from: C₁₋₃ alkylene, —O— and —NR^{a7}—.
   It may be that Ring B is as defined in any one of numbered paragraphs 117 to 184.
188. R¹⁰ at each occurrence is independently selected from: halo, —CN, C₁₋₄ alkyl, C₁₋₄ haloalkyl, —C₁₋₄ alkyl-NR^{a8}R^{b8}, —OH, —OC₁₋₄ alkyl, —OC₁₋₄ haloalkyl, —OC₂₋₄ alkyl-NR^{a8}R^{b8}, —NH₂, —NR^{a6}C₁₋₄ alkyl, —NR^{a6}C₂₋₄ alkyl-OR^{a8}, —NR^{a6}C₂₋₄ alkyl-NR^{a8}R^{b8}, C₁₋₄ haloalkyl, —C(O)C₁₋₄ alkyl, —C(O)C₁₋₄ haloalkyl, —C(O)C₁₋₄ alkyl-NR^{a8}R^{b8}, —COOH, —C(O)OC₁₋₄ alkyl, —C(O)NR^{a6}C₁₋₄ alkyl, —C(O)NR^{a6}C₂₋₄ alkyl-OR^{a8}, —C(O)NR^{a6}C₂₋₄ alkyl-NR^{a8}R^{b8} and Q¹⁰³-L¹⁰³-;
   wherein Q¹⁰³ is a 4 to 6 membered heterocyclyl, wherein said 4 to 6 membered heterocyclyl is selected from: azetidinyl, pyrrolidinyl, piperidinyl, piperazinyl and morpholinyl, each of which is optionally substituted by one or more (e.g. 1 or 2) substituents selected from: halo, ═O, C₁₋₄ alkyl, —OR^{a8}, —NR^{a8}R^{b8} and —C(O)R^{a8};
L¹⁰³ is a bond or is selected from: methylene, —O—, —NH— and —NMe-. It may be that Ring B is as defined in any one of numbered paragraphs 117 to 184.
189. R¹⁰ at each occurrence is independently be selected from: halo, —CN, C₁₋₄ alkyl, C₁-4 haloalkyl, —C₁₋₄ alkyl-NR^{a8}R^{b8}, —NH₂, —NR^{a6}C₁₋₄ alkyl, —NR^{a6}C₂₋₄ alkyl-NR^{a8}R^{b8}, —C(O)C₁₋₄ alkyl, —C(O)C₁₋₄ alkyl-NR^{a8}R^{b8}, —C(O) NHR^{a6}, —C(O) N(R^{a6}) C₁₋₄ alkyl, and —C(O) N(R^{a6}) C₂₋₄ alkyl-NR^{a8}R^{b8}. It may be that Ring B is as defined in any one of numbered paragraphs 117 to 184.
190. R¹⁰ at each occurrence is independently selected from: halo, —CN, —NO₂, ═O, C₁₋₆ alkyl, C₁₋₆ haloalkyl, Q¹-L¹-, —OR¹¹, —S(O)ₓR¹¹ (wherein x is 0, 1, or 2), —NR¹¹R^{a61}, —C(O)R¹¹, —C(O)OR¹¹, —NR^{a61}C(O)R¹¹, and —C(O)NR¹¹R^{a61};
   wherein said C₁₋₆ alkyl is optionally substituted by 1 or more R¹²;
   R¹¹ is independently selected from: H, C₁₋₆ alkyl and C₁₋₆ haloalkyl, wherein said C₁-6 alkyl is optionally substituted by one or more R¹³;
   Q¹ at each occurrence is independently selected from: C₃₋₆ cycloalkyl, 4- to 7-membered heterocyclyl, 4- to 9-membered heterocyclyl-C₁₋₃ alkyl, 5- or 6-membered heteroaryl,
   wherein said C₃₋₆ cycloalkyl, 4- to 7-membered heterocyclyl and 4- to 9-membered heterocyclyl-C₁₋₃ alkyl is optionally substituted by one or more R¹⁴, and
   wherein said 5 or 6 membered heteroaryl is optionally substituted by one or more R¹⁵;
   L¹ is a bond or is selected from —O— or —NR^{a71};
   R¹² at each occurrence is selected from: —NR^{a81}R^{b81}, —C(O)OR^{a81}, —OR^{a81};
   R¹³ at each occurrence is selected from: —NR^{a81}R^{b81} and —OR^{a81};
   R¹⁴ at each occurrence is selected from: halo, ═O, C₁₋₄ alkyl, —NR^{a81}R^{b81} or —OR^{a81};
   R¹⁵ at each occurrence is selected from: halo, C₁₋₄ alkyl, —NR^{a81}R^{b81} or —OR^{a81}; and
R^{a61}, R^{a71}, R^{a81} and R^{b81} are independently selected from H and C₁₋₄ alkyl.
It may be that Ring B is as defined in any one of numbered paragraphs 117 to 184. It may be that in this embodiment, Q¹ at each occurrence is independently selected from: C₃₋₆ cycloalkyl, 4- to 7-membered heterocyclyl, 4- to 7-membered heterocyclyl-C₁₋₃ alkyl-, 5- or 6-membered heteroaryl,
   wherein said C₃₋₆ cycloalkyl, 4- to 7-membered heterocyclyl and 4- to 7-membered heterocyclyl-C₁₋₃ alkyl- is optionally substituted by one or more R¹⁴, and
   wherein said 5 or 6 membered heteroaryl is optionally substituted by one or more R¹⁵
191. R¹⁰ at each occurrence is independently selected from: C₁₋₆ alkyl or Q¹-L¹-,
   wherein said C₁₋₆ alkyl is optionally substituted by 1 or more R¹²;
   Q¹ at each occurrence is independently selected from: 4 to 7 membered heterocyclyl, wherein said heterocyclyl has 1 ring nitrogen atom and optionally one ring atom selected from O, S and N, and wherein Q¹ is optionally substituted by one or two substituents selected from: halo, C₁₋₄ alkyl, —NR^{a81}R^{b81} and —OR^{a81};
   L¹ is a bond or —O—;
   R¹² at each occurrence is selected from: —NR^{a81}R^{b81}, and
   R^{a81} and R^{b81} are independently selected from H and C₁₋₄ alkyl.
It may be that Ring B is as defined in any one of numbered paragraphs 117 to 184.
192. R¹⁰ at each occurrence is independently selected from: Q¹-L¹-,
   Q¹ at each occurrence is independently selected from: 4- to 9-membered heterocyclyl-C₁₋₃ alkyl-, wherein said heterocyclyl has 1 ring nitrogen atom and optionally one ring atom selected from O, S and N; and
   L¹ is a bond.
   It may be that Ring B is as defined in any one of numbered paragraphs 117 to 184.
193. R¹⁰ is selected from: —NR^{a81}R^{b81}, —C₁₋₃ alkyl-NR^{a81}R^{b81}, —NR^{a81}—C₂₋₃ alkyl-NR^{a81}R^{b81}, —C(O)C₁₋₃ alkyl-NR^{a81}R^{b81}, and —C(O)NR^{a81}C₂₋₃ alkyl-NR^{a81}R^{b81}, wherein R^{a81} and R^{b81} are independently selected from H and C₁₋₃ alkyl.
It may be that Ring B is as defined in any one of numbered paragraphs 117 to 184.
194. R¹⁰ is —NR^{a81}R^{b81}, wherein R^{a81} and R^{b81} are independently selected from H and C₁₋₃ alkyl.
It may be that Ring B is as defined in any one of numbered paragraphs 117 to 184.
195. R¹⁰ is selected from: —NR^{a81}R^{b81} and —C₁₋₃ alkyl-NR^{a81}R^{b81}, wherein R^{a81} and R^{b81} are independently selected from H and C₁₋₃ alkyl. It may be that R¹⁰ is —C₁₋₃ alkyl-NR^{a81}R^{b81}, wherein R^{a81} and R^{b81} are independently selected from H and C₁₋₃ alkyl.
It may be that Ring B is as defined in any one of numbered paragraphs 117 to 184.
196. R¹⁰ is selected from:
fluoro, chloro, cyano, nitro, oxo, hydroxy, methyl, ethyl, isopropyl, cyclopropyl, —NH₂, —NH(Me), —N(Me)₂,

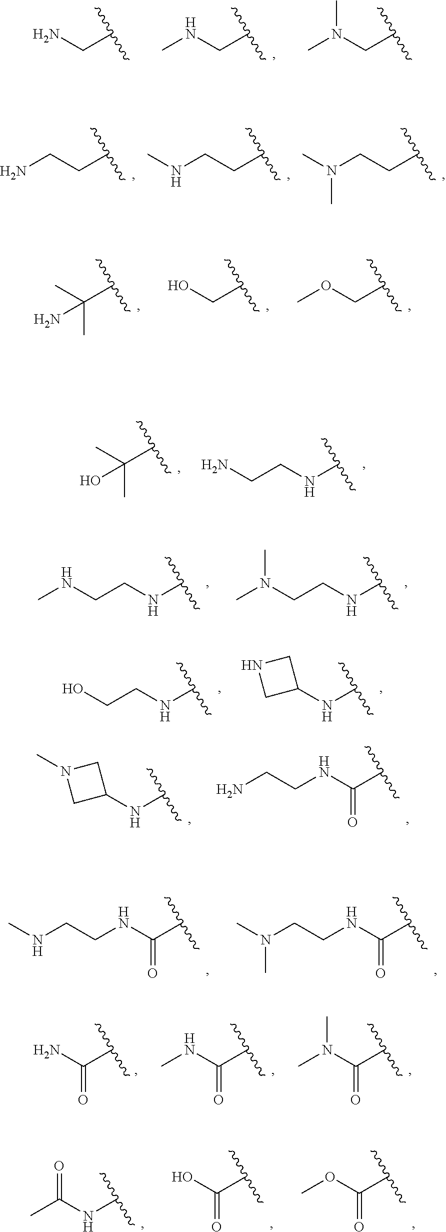

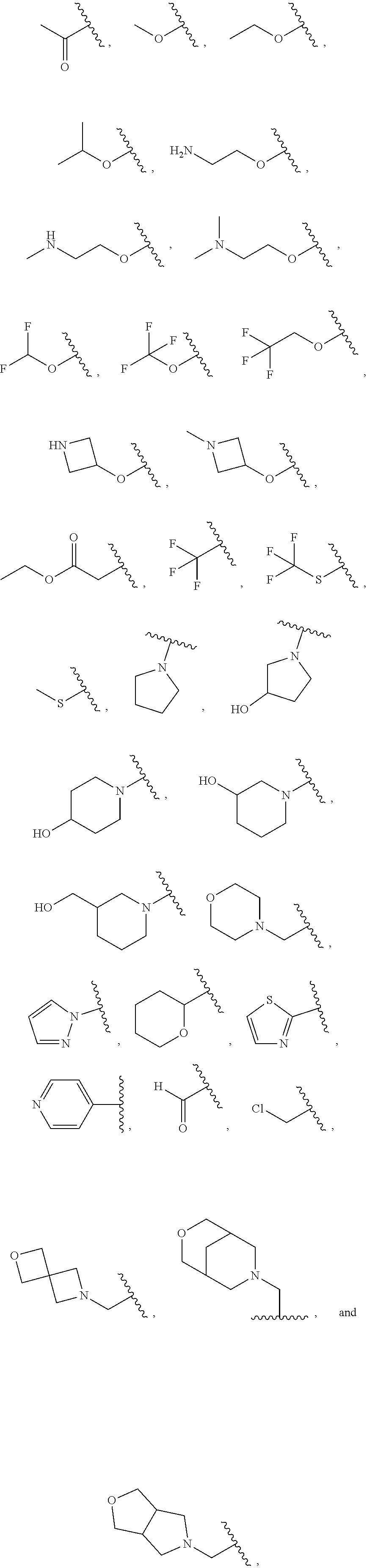

wherein [symbol] indicates the point of attachment to Ring B.
It may be that Ring B is as defined in any one of numbered paragraphs 117 to 184.
197. Ring B is substituted by one R¹⁰ substituent selected from Q¹-L¹-, and Ring B is optionally substituted by one or two additional substituents selected from halo, C₁₋₃ alkyl and C₁₋₃ haloalkyl. It may be that Ring B is as defined in any one of numbered paragraphs 117 to 184.
198. Ring B is substituted by one R¹⁰ substituent selected from Q¹⁰¹-L¹⁰¹-, and Ring B is optionally substituted by one or two additional substituents selected from halo, C₁₋₃ alkyl and C₁₋₃ haloalkyl,
   wherein Q¹⁰¹ is selected from C₃₋₆ cycloalkyl, 4 to 6 membered heterocyclyl, 5- or 6-membered heteroaryl,
   wherein said C₃₋₆ cycloalkyl and 4 to 6 membered heterocyclyl is optionally substituted by one or more (e.g. 1 or 2) substituents selected from: halo, ═O, C₁₋₄ alkyl, —OR^{a8}, —NR^{a8}R^{b8}, —C(O)R^{a8} and —S(O)₂R^{a8}
   wherein said 5- or 6-membered heteroaryl is optionally substituted by one or more (e.g. 1 or 2) substituents selected from: halo, C₁₋₄ alkyl, —OR^{a10}, —NR^{a10}R^{b10}, —C(O)R^{a10} and —S(O)₂R^{a1}; and
   L¹⁰¹ is a bond or is selected from: C₁₋₃ alkylene, —O— and —NR^{a7}—.
   It may be that Ring B is as defined in any one of numbered paragraphs 117 to 184.
199. Ring B is substituted by one R¹⁰ substituent selected from Q¹⁰²-L¹⁰²-, and Ring B is optionally substituted by one or two additional substituents selected from halo, C₁₋₃ alkyl and C₁₋₃ haloalkyl,
   wherein Q¹⁰² is selected from 4 to 6 membered heterocyclyl and 5- or 6-membered heteroaryl,
   wherein said 4 to 6 membered heterocyclyl is selected from: azetidinyl, pyrrolidinyl, piperidinyl, piperazinyl and morpholinyl, each of which is optionally substituted by one or more (e.g. 1 or 2) substituents selected from: halo, ═O, C₁₋₄ alkyl, —OR^{a8}, —NR^{a8}R^{b8} and —C(O)R^{a8},
   wherein said 5- or 6-membered heteroaryl is selected from: pyrrolyl, imidazolyl, oxazolyl, isoxazolyl, thiazolyl, isothiazolyl, pyrazolyl, oxadiazolyl, triazolyl, pyridyl, pyrimidyl, pyrazinyl and pyridazinyl, each of which is optionally substituted by one or more (e.g. 1 or 2) substituents selected from: halo, C₁₋₄ alkyl, —OR^{a10} and —NR^{a10}R^{b10}.
   L¹⁰² is a bond or is selected from: C₁₋₃ alkylene, —O— and —NR^{a7}.
   It may be that Ring B is as defined in any one of numbered paragraphs 117 to 184.
200. Ring B is substituted by one R¹⁰ substituent selected from Q¹⁰³-L¹⁰³-, and Ring B is optionally substituted by one or two additional substituents selected from halo, C₁₋₃ alkyl and C₁₋₃ haloalkyl,
   wherein Q¹⁰³ is a 4- to 6-membered heterocyclyl, wherein said 4- to 6-membered heterocyclyl is selected from: azetidinyl, pyrrolidinyl, piperidinyl, piperazinyl and morpholinyl, each of which is optionally substituted by one or more (e.g. 1 or 2) substituents selected from: halo, ═O, C₁₋₄ alkyl, —OR^{a8}, —NR^{a8}R^{b8} and —C(O)R^{a8};
   L¹⁰³ is a bond or is selected from: methylene, —O—, —NH— and —NMe-.
   It may be that Ring B is as defined in any one of numbered paragraphs 117 to 184.
201. Ring B is selected from:

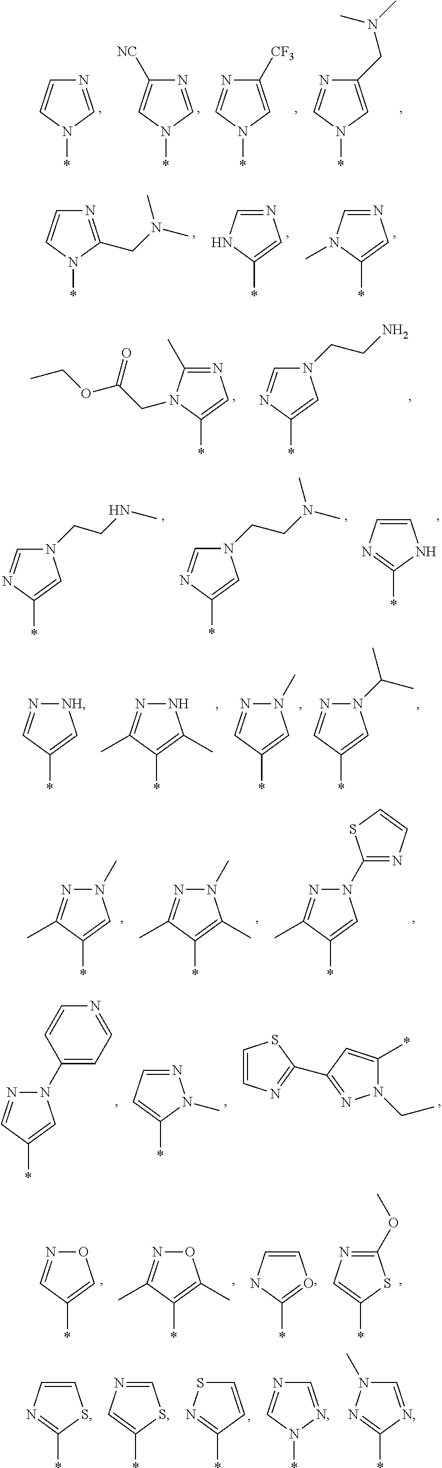

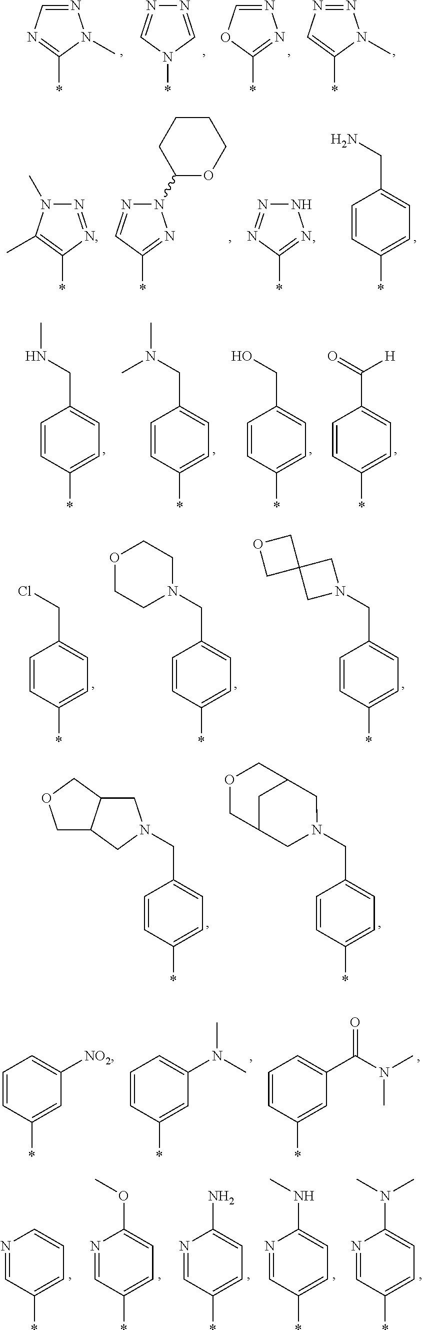

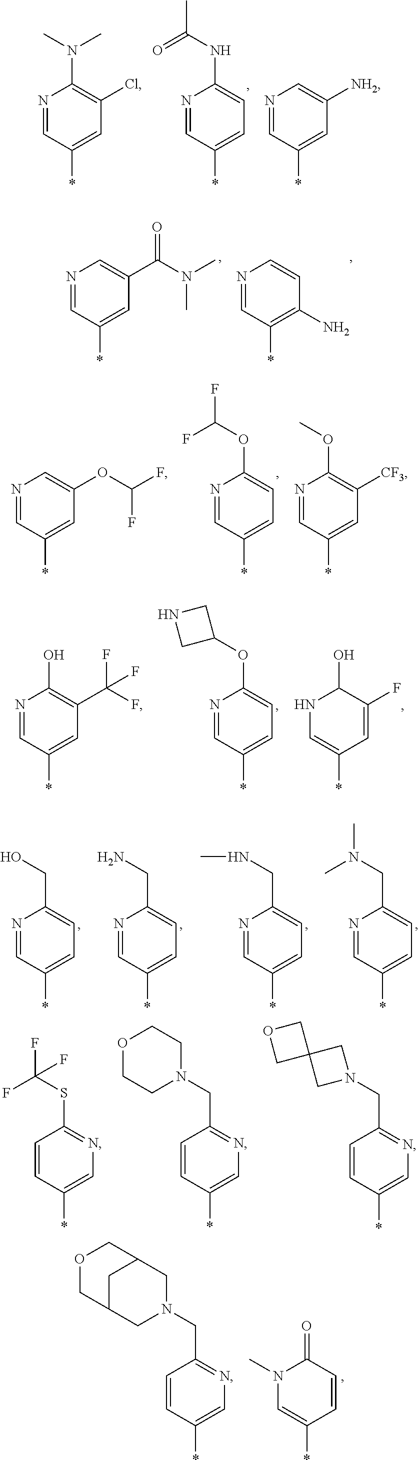

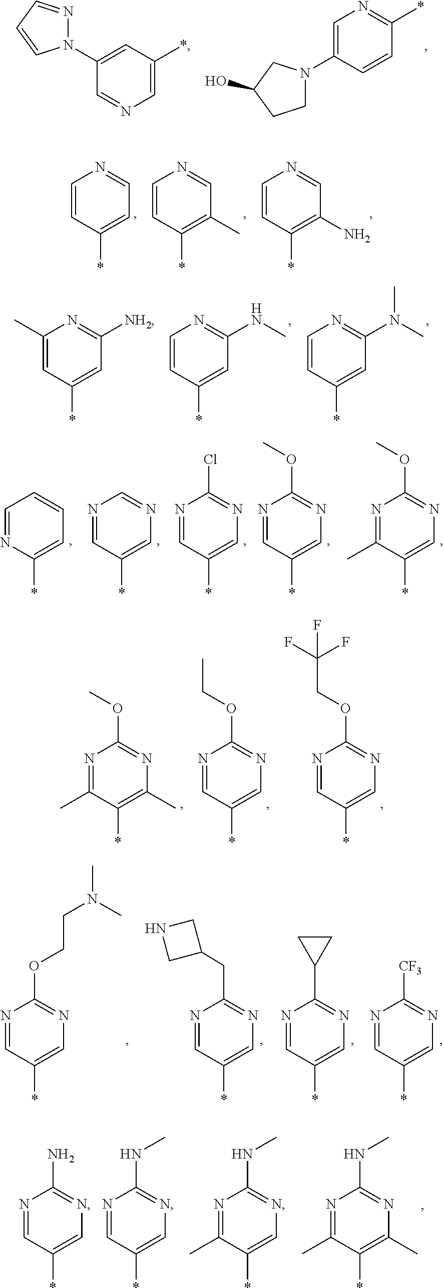

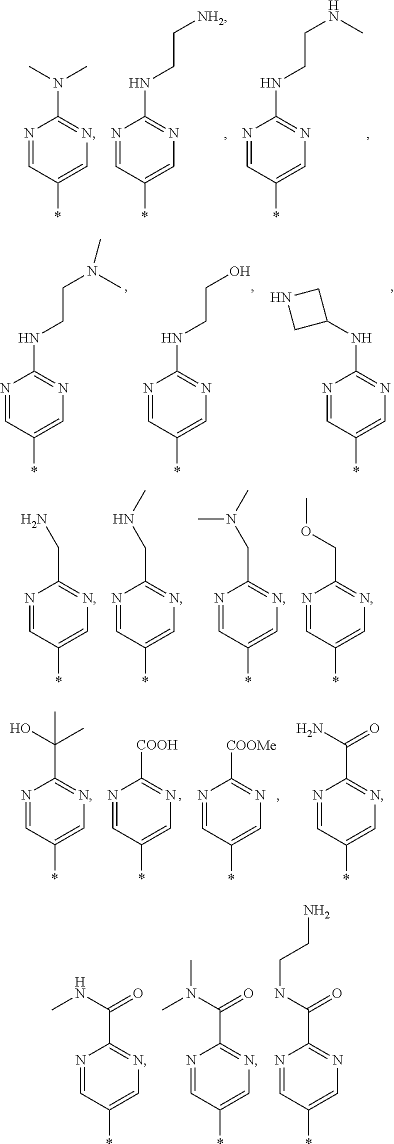

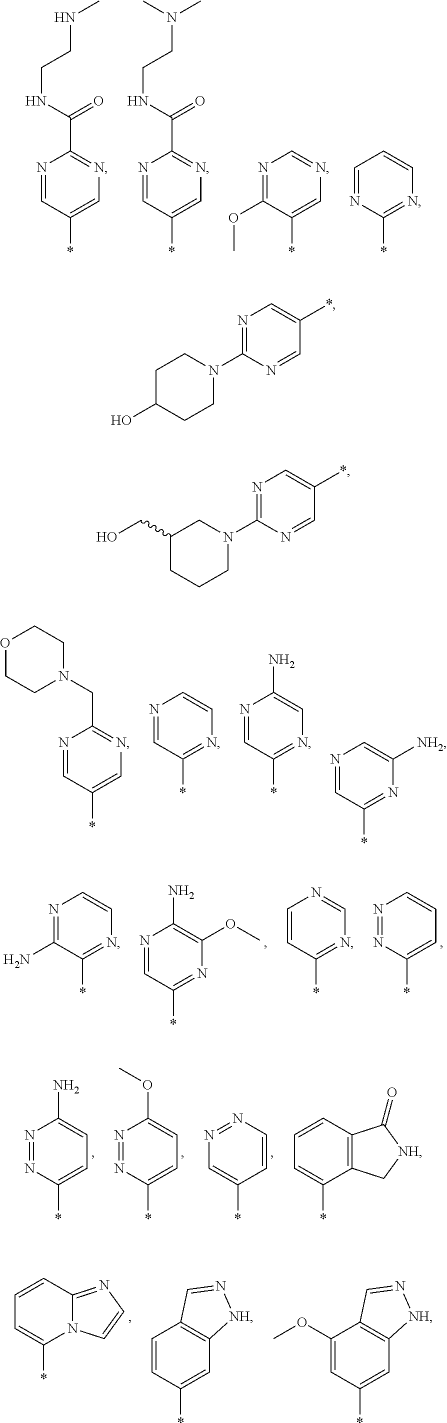

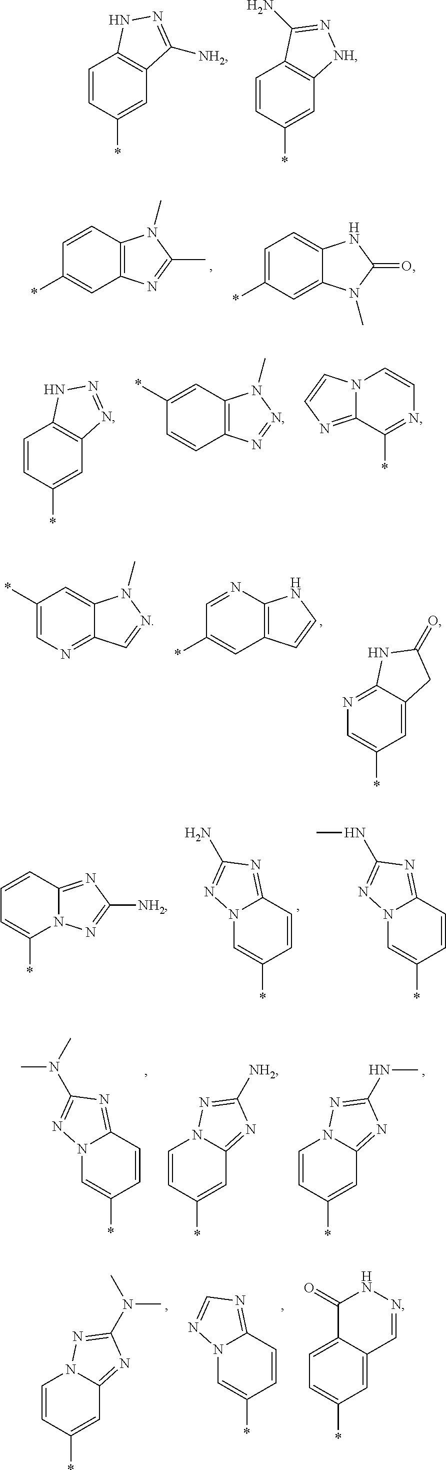

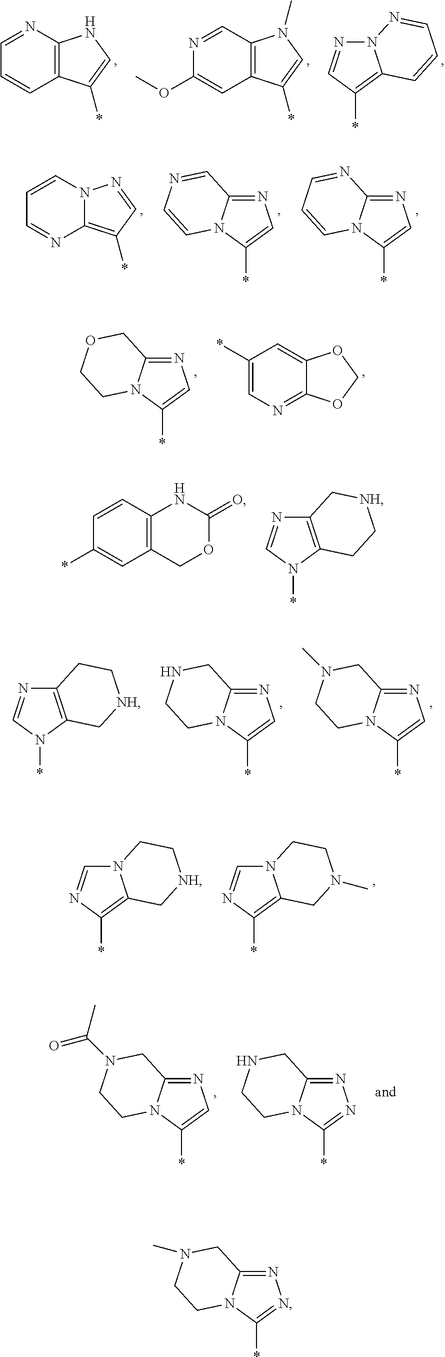

wherein * indicates the point of attachment to the remainder of the compound in Formula (I).
202. Ring B is selected from:

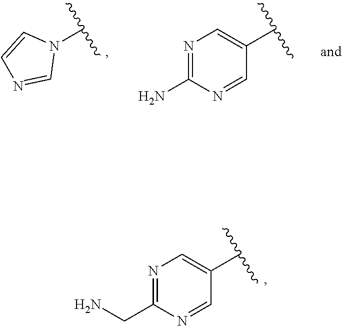 [SMILES: *n1ccnc1 ; *c1cnc(N)nc1 ; *c1cnc(CN)nc1]

wherein [symbol] indicates the point of attachment to the remainder of the compound in Formula (I).
203. Ring B is selected from:

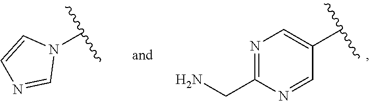 [SMILES: *n1ccnc1 ; *c1cnc(CN)nc1]

wherein [symbol] indicates the point of attachment to the remainder of the compound in Formula (I).
204. In compounds of formula (I), X¹ and X³ are N, and X² is CH.

In certain embodiments the compound of the invention is a compound of the formula (I), (II), (III), (IV), (VII), (VIII), (IX), (XII), (XIII), (XIV), (XV), (XVIII), (XIX), (XX), (XXIII), (XXIV), (XXV), (XXVI), (XXVII), (XXVIII), (XXIX), (XXX) or (XXXI), or a pharmaceutically acceptable salt thereof, wherein R¹ is selected from: phenyl and a 5- or 6-membered heteroaryl containing at least one ring nitrogen, wherein R¹ is optionally substituted by one or more substituents selected from: halo, C₁₋₄ alkyl, C₁₋₄ haloalkyl, —OR^{a1}, —SR^{a1} and —NR^{a1}R^{b1}.

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX) or (XXXI), or a pharmaceutically acceptable salt thereof, wherein R³ or —OR⁴¹ are selected from —OMe and —OCH(CH₃)₂. Thus, it may be that R³ or —OR⁴¹ is —OMe. It may be that R³ or —OR⁴¹ is —OCH(CH₃)₂.

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX) or (XXXI), or a pharmaceutically acceptable salt thereof, wherein X¹ is CR⁷ and X² is N. For example, X¹ is CR⁷, wherein R⁷ is selected from: H, halo and C₁₋₄ alkyl; and X² is N. For example wherein X¹ is CH. and X² is N. Suitably in these embodiments R³ or —OR⁴¹ are selected from —OMe and —OCH(CH₃)₂ (e.g. R³ or —OR⁴¹ is —OMe).

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX) or (XXXI), or a pharmaceutically acceptable salt thereof, wherein X¹ is N and X² is CR⁷. For example, wherein X¹ is N and X² is CR⁷, wherein R⁷ is selected from: H, halo and C₁₋₄ alkyl. For example wherein X¹ is N and X² is CH. Suitably in these embodiments R³ or —OR⁴¹ are selected from —OMe and —OCH(CH₃)₂ (e.g. R³ or —OR⁴¹ is —OMe).

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX) or (XXXI), or a pharmaceutically acceptable salt thereof, wherein X¹ and X² are CR⁷. In these embodiments it may be that X¹ is CH and X² is CR⁷. It may be that X¹ is CR⁷ and X² is CH. In these embodiments it may be that R⁷ is selected from: H, halo and C₁₋₄ alkyl. Thus it may be that X¹ and X² are CH. Suitably in these embodiments R³ or —OR⁴¹ are selected from —OMe and —OCH(CH₃)₂ (e.g. R³ or —OR⁴¹ is —OMe).

In certain embodiments the compound of the invention is a compound of the formula (I), (II), (III), (IV), (V), (VI), (XII), (XIII), (XIV), (XV), (XVI), (XVII), (XXIII), (XXIV), (XXVI), (XXVII), (XXIX) or (XXX), or a pharmaceutically acceptable salt thereof, wherein X¹, X² and X³ are CH. Suitably in these embodiments R³ or —OR⁴¹ are selected from —OMe and —OCH(CH₃)₂ (e.g. R³ or —OR⁴¹ is —OMe).

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or (XXII), or a pharmaceutically acceptable salt thereof, wherein Ring B is as defined in any one of numbered paragraphs 117 to 184, 201 or 202.

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or (XXII), or a pharmaceutically acceptable salt thereof, wherein Ring B is as defined in any one of numbered paragraphs 117 to 184, 201 or 202; and
R³ or —OR⁴¹ are selected from —OMe and —OCH(CH₃)₂ (e.g. R³ or —OR⁴¹ is —OMe).

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or (XXII), or a pharmaceutically acceptable salt thereof, wherein Ring B is as defined in any one of numbered paragraphs 117 to 184, 201 or 202; and X¹ and X² are both CH. Suitably in these embodiments R³ or —OR⁴¹ are selected from —OMe and —OCH(CH₃)₂ (e.g. R³ or —OR⁴¹ is —OMe).

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or (XXII), or a pharmaceutically acceptable salt thereof, wherein Ring B is as defined in any one of numbered paragraphs 117 to 184, 201 or 202; and X¹ is N and X² is CH. Suitably in these embodiments R³ or —OR⁴¹ are selected from —OMe and —OCH(CH₃)₂ (e.g. R³ or —OR⁴¹ is —OMe).

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or (XXII), or a pharmaceutically acceptable salt thereof, wherein Ring B is as defined in any one of numbered paragraphs 117 to 184, 201 or 202; and X¹ is CH and X² is N. Suitably in these embodiments R³ or —OR⁴¹ are selected from —OMe and —OCH(CH₃)₂ (e.g. R³ or —OR⁴¹ is —OMe).

In certain embodiments the compound of the invention is a compound of the formula (I), (XII), (XXIII), (XXIV), (XXV), (XXVI), (XXVII), (XXVIII), (XXIX), (XXX) or (XXXI), or a pharmaceutically acceptable salt thereof, wherein Ring A is as defined in any one of numbered paragraphs 37 and 38. Suitably in these embodiments R³ or —OR⁴¹ are selected from —OMe and —OCH(CH₃)₂ (e.g. R³ or —OR⁴¹ is —OMe). In these embodiments it may be that X¹ and X² are both CH. In these embodiments it may be that X¹ is CH and X² is N. In these embodiments it maybe that X¹ is N and X² is CH.

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or (XXII), or a pharmaceutically acceptable salt thereof, wherein Ring B is optionally substituted by one or two R¹⁰ and wherein R¹⁰ is as defined in any one of numbered paragraphs 185 to 196. Suitably in these embodiments R³ or —OR⁴¹ are selected from —OMe and —OCH(CH₃)₂ (e.g. R³ or —OR⁴¹ is —OMe). Thus in this embodiment it may be that Ring B is unsubstituted. It may in this embodiment that Ring B is substituted by one or two R¹⁰, wherein R¹⁰ is as defined in any one of numbered paragraphs 185 to 196.

In certain embodiments the compound of the invention is a compound of the formula (XXIII), (XXIV), (XXV), (XXVI), (XXVII), (XXVIII), (XXIX), (XXX) or (XXXI), or a pharmaceutically acceptable salt thereof, wherein R¹⁰ is as defined in any one of numbered paragraphs 185 to 196. Suitably in these embodiments R³ or —OR⁴¹ are selected from —OMe and —OCH(CH₃)₂ (e.g. R³ or —OR⁴¹ is —OMe). Suitably in these embodiments p (when present) is 0, 1 or 2.

In certain embodiments the compound of the invention is a compound of the formula (I), (II), (III), (IV), (VII), (VIII), (IX), (XIII), (XIV), (XV), (XVIII), (XIX) or (XX), or a pharmaceutically acceptable salt thereof, wherein R¹ is as defined in any one of numbered paragraphs 4 to 21; and R² is methyl. Suitably in these embodiments R³ or —OR⁴¹ are selected from —OMe and —OCH(CH₃)₂ (e.g. R³ or —OR⁴¹ is —OMe).

In certain embodiments the compound of the invention is a compound of the formula (I), (II), (III), (IV), (V), (VII), (VIII), (IX), (X), (XIII), (XIV), (XV), (XVI), (XVIII), (XIX), (XX) or (XXI), or a pharmaceutically acceptable salt thereof, wherein R² is not H. Thus it may be that R² is as defined in and one of numbered paragraphs 26, 28, or 30 to 32.

In certain embodiments the compound of the invention is a compound of the formula (I), (XII), (XXIII), (XXIV), (XXV), (XXVI), (XXVII), (XXVIII), (XXIX), (XXX) or (XXXI), or a pharmaceutically acceptable salt thereof, wherein Ring A is as defined in numbered paragraphs 37 or 38. It may be that Ring A is as defined in numbered paragraph 37 and R³ is as defined in any one of numbered paragraphs 39 to 81. For example wherein Ring A is as defined in numbered paragraph 37 and R³ is methoxy.

In certain embodiments the compound of the invention is a compound of the formula (I), (II), (III), (IV), (VII), (VIII), (IX), (XIII), (XIV), (XV), (XVIII), (XIX) or (XX), or a pharmaceutically acceptable salt thereof, wherein R² is as defined in any one of numbered paragraphs 26 to 32; and R¹ is selected from phenyl or 4-fluorophenyl. Suitably in these embodiments R³ or —OR⁴¹ are selected from —OMe and —OCH(CH₃)₂ (e.g. R³ or —OR⁴¹ is —OMe).

Suitably in the compounds of the formula (I), (IV), (IX), (XII), (XV), (XX), (XXIII), (XXIV). (XXV), (XXVI), (XXVII), (XXVIII), (XXIX), (XXX) and (XXXI), or a pharmaceutically acceptable salt thereof, when Ring A is of the formula A3 then R² is not —OR^{a2}, —SR^{a2} and —NR^{a2}R^{b2}. For example in these embodiments it may be that when Ring A is A3, R² is selected from halo, C₁₋₄ alkyl and C₁₋₄ haloalkyl. it may be that when Ring A is A3, R² is H.

In certain embodiments the compound of the invention is a compound of the formula (XXIII), (XXIV), (XXV), (XXVI), (XXVII), (XXVIII), (XXIX), (XXX) or (XXXI), or a pharmaceutically acceptable salt thereof, wherein R¹⁰ is —NR^{a81}R^{b81} and —C₁₋₃ alkyl-NR^{a81}R^{b81}, wherein R^{a81} and R^{b81} are independently selected from H and C₁₋₃ alkyl. It may be that R¹⁰ is —CH₂—NR^{a81}R^{b81}, wherein R^{a81} and R^{b81} are independently selected from H and C₁₋₃ alkyl. In this embodiment it may be that R³ or —OR⁴¹ is selected from: —OMe, —OCH(CH₃)₂ and -Ocyclobutyl. For example, R³ or —OR⁴¹ is selected from: —OMe and —OCH(CH₃)₂.

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI) or (XXII), or a pharmaceutically acceptable salt thereof, wherein Ring B is substituted with one R¹⁰ substituent selected from —NR^{a81}R^{b81} and —C₁₋₃ alkyl-NR^{a81}R^{b81}, wherein R^{a81} and R^{b81} are independently selected from H and C₁₋₃ alkyl; and optionally one or two substituents selected from halo and C₁₋₄ alkyl. It may be that Ring B is substituted with only one R¹⁰ substituent selected from —NR²⁸¹R^{b81} and —C₁₋₃ alkyl-NR^{a81}R^{b81}, wherein R^{a81} and R^{b81} are independently selected from H and C₁₋₃ alkyl. It may be that R¹⁰ is —CH₂—NR^{a81}R^{b81}, wherein R^{a81} and R^{b81} are independently selected from H and C₁₋₃ alkyl. In this embodiment it may be that R³ or —OR⁴¹ is selected from: —OMe, —OCH(CH₃)₂ and —O-cyclobutyl. For example, R³ or —OR⁴¹ is selected from: —OMe and —OCH(CH₃)₂.

In another embodiment the compound of formula (I) is a compound of the formula (II):

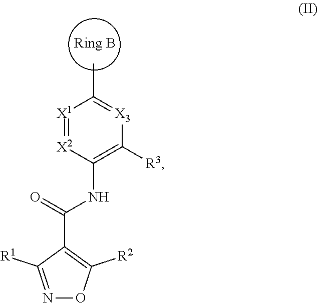

or a pharmaceutically acceptable salt thereof, wherein:
R¹ is selected from: phenyl and a 5- or 6-membered heteroaryl containing at least one nitrogen atom, wherein R¹ is optionally substituted by one or more substituents selected from: halo, C₁₋₄ alkyl, C₁₋₄ haloalkyl, —OR^{a1}, —SR^{a1} and —NR^{a1}R^{b1};
R² is selected from: H, halo, C₁₋₄ alkyl and C₁₋₄ haloalkyl, —OR^{a2}, —SR^{a2} and —NR^{a2}R^{b2}
   wherein the C₁₋₄ alkyl is optionally substituted by one or more substituents selected from: halo, —OR^{a3}, —SR^{a3} and —NR^{a3}R^{b3},
R³ is selected from: C₁₋₄ alkyl, C₁₋₄ haloalkyl, C₃₋₆ cycloalkyl, C₃₋₆ cycloalkyl-C₁₋₃ alkyl-, —OR⁴, —NR⁵R⁶, —SR⁵, 4- to 7-membered heterocyclyl containing 1 or more ring oxygen atoms, and 4- to 7-membered heterocyclyl-C₁₋₃ alkyl-containing 1 or more ring oxygen atoms;
   R⁴ and R⁵ are independently selected from: H, C₁₋₄ alkyl, C₁₋₄ haloalkyl, C₃₋₆ cycloalkyl and C₃₋₆ cycloalkyl-C₁₋₃ alkyl-, 4- to 7-membered heterocyclyl containing 1 or more ring oxygen atoms, and 4- to 7-membered heterocyclyl-C₁₋₃ alkyl-containing 1 or more ring oxygen atoms;
   R⁶ is selected from: H, C₁₋₄ alkyl and C₁₋₄ haloalkyl;
X¹, X² and X³ are independently selected from: N and CR⁷;
   R⁷ is at each occurrence is independently selected from: H, halo, —CN, C₁₋₄ alkyl, C₁₋₄ haloalkyl, —OR⁸, —NR⁸R⁹ and —S(O)₂R⁸ (wherein x is 0, 1, or 2);
   R⁸ and R⁹ are each independently selected from: H, C₁₋₄ alkyl and C₁₋₄ haloalkyl;
   wherein any C₁₋₄ alkyl in any of R⁷, R⁸ or R⁹ is optionally substituted by one or more substituents selected from: halo, —CN, —OR^{a5}, —S(O)ₓR^{a5} (wherein x is 0, 1, or 2) and —NR^{a5}R^{b5};
Ring B is selected from phenyl and 5- to 12-membered heteroaryl optionally substituted with one or more R¹⁰, wherein
   R¹⁰ at each occurrence is independently selected from: halo, —CN, —NO₂, ═O, C₁₋₆ alkyl, C₂₋₆ alkenyl, C₂₋₆ alkynyl, C₁₋₆ haloalkyl, Q¹-L¹-, —OR¹¹, —S(O)ₓR¹¹ (wherein x is 0, 1, or 2), —NR¹¹R^{a6}, —C(O)R¹¹, —OC(O)R¹¹, —C(O)OR¹¹, —NR^{a6}C(O)R¹¹, —NR^{a6}C(O)OR¹¹, —C(O)NR¹¹R^{a6}, —OC(O)NR¹¹R^{a6}, —NR^{a6}SO₂R¹¹, —SO₂NR¹¹R^{a6} and —NR^{a6}C(O)NR¹¹R^{a6}
   wherein said C₁₋₆ alkyl, C₂₋₆ alkenyl and C₂₋₆ alkynyl is optionally substituted by 1 or more R¹²;
   R¹¹ is independently selected from: H, C₁₋₆ alkyl and C₁₋₆ haloalkyl, wherein said C₁₋₆ alkyl is optionally substituted by one or more R¹³;
   Q¹ at each occurrence is independently selected from: C₃₋₆ cycloalkyl, C₃₋₆ cycloalkyl-C₁₋₃ alkyl-, 4- to 7-membered heterocyclyl, 4- to 9-membered heterocyclyl-C₁₋₃ alkyl-, phenyl, phenyl-C₁₋₃ alkyl-, 5- or 6-membered heteroaryl and 5- or 6-membered heteroaryl-C₁₋₃ alkyl-,
   wherein said C₃₋₆ cycloalkyl, C₃₋₆ cycloalkyl-C₁₋₃ alkyl-, 4- to 7-membered heterocyclyl and 4- to 9-membered heterocyclyl-C₁₋₃ alkyl- is optionally substituted by one or more R¹⁴, and
   wherein said phenyl, phenyl-C₁₋₃ alkyl, 5- or 6-membered heteroaryl and 5- or 6-membered heteroaryl-C₁₋₃ alkyl is optionally substituted by one or more R¹⁵;
   L¹ is a bond or is selected from —O—, —S(O)ₓ-(wherein x is 0, 1, or 2), —NR^{a7}—, —C(O)—, —OC(O)—, —C(O)O—, —NR^{a7}C(O)—, —C(O)NR^{a7}—, —NR^{a7}C(O)O—, —OC(O)NR^{a7}—, —NR^{a7}SO₂—, —SO₂NR^{a7}— and —NR^{a7}C(O)NR^{a7}—;
   R¹², R¹³ and R¹⁴ are at each occurrence independently selected from: halo, ═O, —CN, —NO₂, C₁₋₄ alkyl, C₁₋₄ haloalkyl, —OR^{a8}, —S(O)₂R^{a8}, —NR^{a8}R^{b8}, —C(O)R^{a8}, —OC(O)R^{a8}, —C(O)OR^{a8}, —NR^{a8}C(O)R^{b8}, —C(O)NR^{a8}R^{b8}, —NR^{a8}C(O)OR^{b8}, —OC(O)NR^{a8}R^{b8}, —NR^{a8}SO₂R^{b8} and —SO₂NR^{a8}R^{b8};
   wherein said C₁₋₄ alkyl is optionally substituted by 1 or 2 substituents selected from: halo, —CN, —OR^{a9}, —NR^{a9}R^{b9} and —SO₂R^{a9};
   R¹⁵ is at each occurrence independently selected from: halo, ═O, —CN, —NO₂, C₁₋₄ alkyl, C₁₋₄ haloalkyl, —OR^{a10}, —S(O)₂R^{a10}, —NR^{a10}R^{b10}, —C(O)R^{a10}, —OC(O)R^{a10}, —C(O)OR^{a10}, —NR^{a10}C(O)R^{b10}, —C(O)NR^{a10}R^{b10}, —NR^{a10}C(O) OR^{b10}, —OC(O)NR^{a10}R^{b10}, —NR^{b10}SO₂R^{a10} and —SO₂NR^{a10}R^{b10};
   wherein said C₁₋₄ alkyl is optionally substituted by 1 or 2 substituents selected from: halo, —CN, —OR^{a11}, —NR^{a11}R^{b11} and —SO₂R^{a11}; R^{a1}, R^{b1}, R^{a2}, R^{b2}, R^{a3}, R^{b3}, R^{a4}, R^{b4}, R^{a5}, R^{b5}, R^{a6}, R^{a7}, R^{a8}, R^{b8}, R^{a9}, R^{b9}, R^{a10}, R^{b10}, R^{a11} and R^{b11} are at each occurrence independently selected from: H, C₁₋₄ alkyl and C₁₋₄ haloalkyl,
   or any —NR^{a1}R^{b1}, —NR^{a2}R^{b2}, —NR^{a3}R^{b3}, —NR^{a4}R^{b4}, —NR^{a5}R^{b5}, —NR^{a8}R^{b8}, —NR^{a9}R^{b9}, —NR^{a10}R^{b10}, —NR^{a11}R^{b11}, —NR⁵R⁶, —NR⁸R⁹ or —NR¹¹R^{a6} within a substituent may form a 4 to 6 membered heterocyclyl, wherein said 4 to 6 membered heterocyclyl is optionally substituted by one or more substituents selected from: halo, ═O, C₁₋₄ alkyl and C₁₋₄ haloalkyl.

In this embodiment, it may be that R³ is selected from: C₁₋₄ alkyl, C₁₋₄ haloalkyl, C₃₋₆ cycloalkyl, C₃₋₆ cycloalkyl-C₁₋₃ alkyl- and —OR⁴. For example, it may be that R³ is —OR⁴.

In this embodiment, it may be that R⁴ is selected from: H, C₁₋₄ alkyl, C₁₋₄ haloalkyl, C₃₋₆ cycloalkyl, C₃₋₆ cycloalkyl-C₁₋₃ alkyl-, 4- to 6-membered heterocyclyl containing 1 or ring oxygen atom, and 4- to 6-membered heterocyclyl-C₁₋₃ alkyl-containing 1 ring oxygen atom.

In this embodiment, it may be that Q¹ at each occurrence is independently selected from: C₃₋₆ cycloalkyl, C₃₋₆ cycloalkyl-C₁₋₃ alkyl-, 4- to 7-membered heterocyclyl, 4- to 9-membered heterocyclyl-C₁₋₃ alkyl-, phenyl, phenyl-C₁₋₃ alkyl-, 5- or 6-membered heteroaryl and 5- or 6-membered heteroaryl-C₁₋₃ alkyl-,
wherein said C₃₋₆ cycloalkyl, C₃₋₆ cycloalkyl-C₁₋₃ alkyl, 4- to 7-membered heterocyclyl and 4- to 7-membered heterocyclyl-C₁₋₃ alkyl- is optionally substituted by one or more R¹⁴, and
wherein said phenyl, phenyl-C₁₋₃ alkyl, 5- or 6-membered heteroaryl and 5- or 6-membered heteroaryl-C₁₋₃ alkyl- is optionally substituted by one or more R¹⁵.

It may be in this embodiment that Ring B is as defined in any one of numbered paragraphs 117 to 184, 201 or 202.

In this embodiment it may be that R³ is selected from: —OMe, —OCH(CH₃)₂ and —O-cyclobutyl. Thus it may be that R³ is —OMe. It may be that R³ is —OCH(CH₃)₂. It may be that R³ is —O-cyclobutyl.

In this embodiment it may be that R³ is selected from: —OMe, —OCH(CH₃)₂ and oxetan-3-yloxy-.

In another embodiment the compound of formula (I) is a compound of the formula (II):

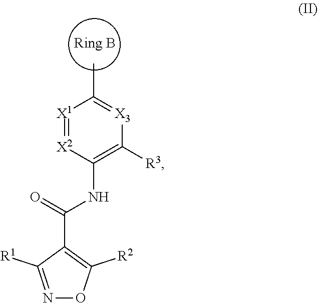

or a pharmaceutically acceptable salt thereof, wherein:
R¹ is phenyl, optionally substituted by one or two substituents selected from halo;
R² is C₁₋₄ alkyl;
R³ is —OR⁴;
R⁴ is selected from: C₁₋₄ alkyl and C₃₋₆ cycloalkyl;
X¹ and X² are CH;
X³ is N;
Ring B is selected from:

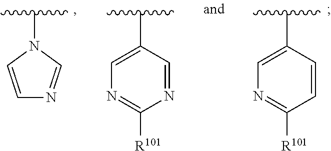

R¹⁰¹ is independently selected from: H, C₁₋₄ alkyl, —C₁₋₃ alkyl-NR^{a82}R^{b82}, —NR^{a82}R^{b82} and Q¹-L¹-,
Q¹ is selected from: azetidinyl, pyrrolidinyl, piperidinyl, piperazinyl and morpholinyl, each of which is optionally substituted by 1 or 2 substituents selected from: halo, C₁₋₄ alkyl and ═O;
   L¹ is a bond or is selected from: —O—, —NH— and —N(C₁₋₃ alkyl)-; and
R^{a82} and R^{b82} are independently selected from: H and C₁₋₄ alkyl.

In this embodiment it may be that R³ is selected from: —OMe, —OCH(CH₃)₂ and —O-cyclobutyl. Thus it may be that R³ is —OMe. It may be that R³ is —OCH(CH₃)₂. It may be that R³ is —O-cyclobutyl.

In this embodiment it may be that R¹⁰¹ is selected from: —NR^{a82}R^{b82}, —C₁₋₃ alkyl-NR^{a82}R^{b82}, and Q¹-L¹-, wherein Q¹ is azetidinyl which is optionally substituted by 1 substituent selected from: C₁₋₄ alkyl; and L¹ is a bond or is selected from: —O— and —NH—. Thus it may be that R¹⁰¹ is selected from: —NH₂, —N(Me) H, —N(Me)₂, —CH₂—NH₂, —CH₂—N (Me) H, —CH₂—N(Me)₂, azetidin-3-yl-O—, 1-methylazetidin-3-yl-O—, azetidin-3-yl-NH— and 1-methylazetidin-3-yl-NH—. For example it may be that R¹⁰¹ is —CH₂—NH₂.

In this embodiment it may be that:
R¹ is phenyl or 4-fluorophenyl;
R² is methyl;
R³ is —OMe or —CH(CH₃)₂;
X¹ and X² are CH;
X³ is N;
Ring B is selected from:

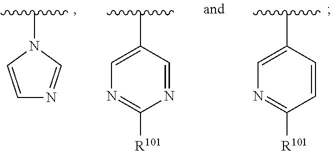

R¹⁰¹ is independently selected from: from: —NR^{a82}R^{b82}, —C₁₋₃ alkyl-NR^{a82}R^{b82}, and Q¹-L¹-;
Q¹ is azetidinyl which is optionally substituted by 1 substituent selected from: C₁₋₄ alkyl;
L¹ is a bond or is selected from: —O— and —NH—; and
R^{a82} and R^{b82} are independently selected from: H and C₁₋₄ alkyl.

In another embodiment there is provided a compound selected from Compound List 1, or a pharmaceutically acceptable salt thereof:

### Compound List 1

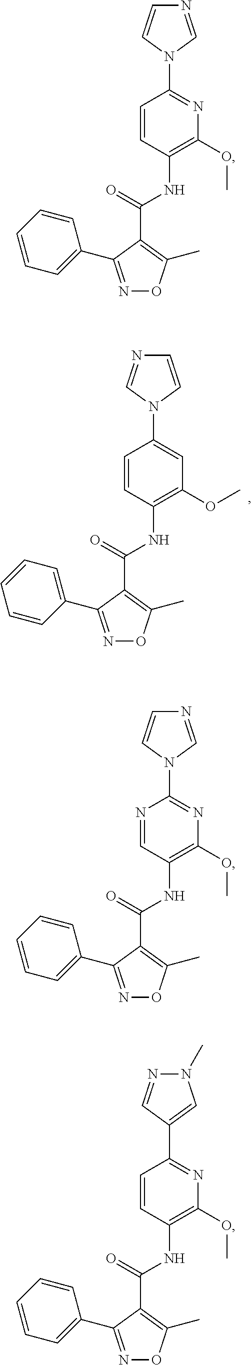 [SMILES: COc1nc(-n2ccnc2)ccc1NC(=O)c1c(-c2ccccc2)noc1C ; COc1cc(-n2ccnc2)ccc1NC(=O)c1c(-c2ccccc2)noc1C ; COc1nc(-n2ccnc2)ncc1NC(=O)c1c(-c2ccccc2)noc1C ; COc1nc(-c2cnn(C)c2)ccc1NC(=O)c1c(-c2ccccc2)noc1C]

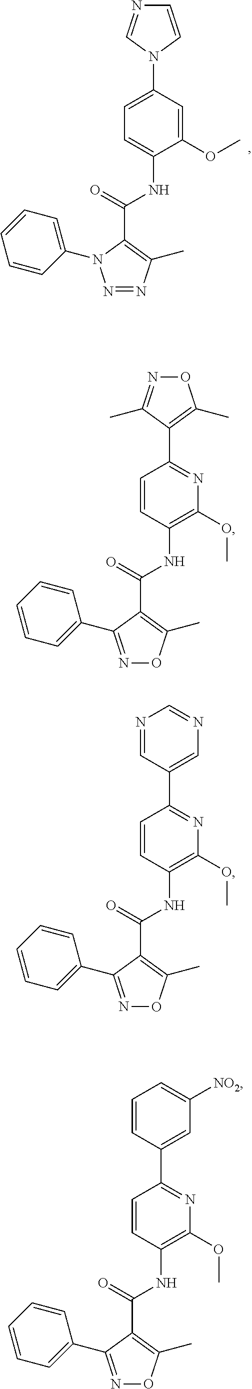

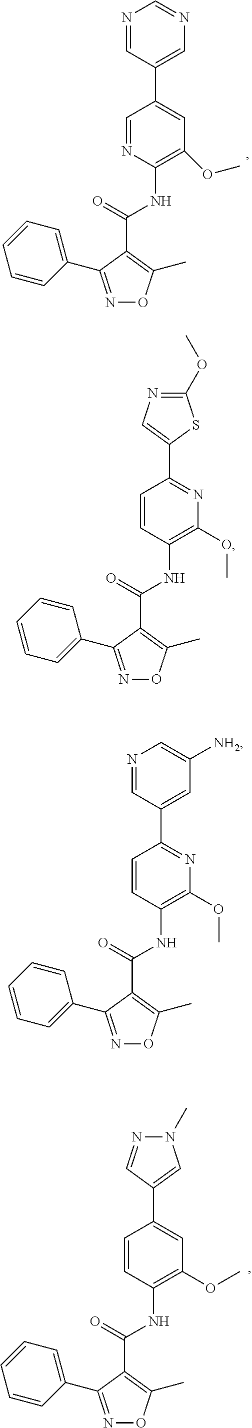 [SMILES: COc1cc(-c2cncnc2)cnc1NC(=O)c1c(-c2ccccc2)noc1C ; COc1ncc(-c2ccc(NC(=O)c3c(-c4ccccc4)noc3C)c(OC)n2)s1 ; COc1nc(-c2cncc(N)c2)ccc1NC(=O)c1c(-c2ccccc2)noc1C ; COc1cc(-c2cnn(C)c2)ccc1NC(=O)c1c(-c2ccccc2)noc1C]

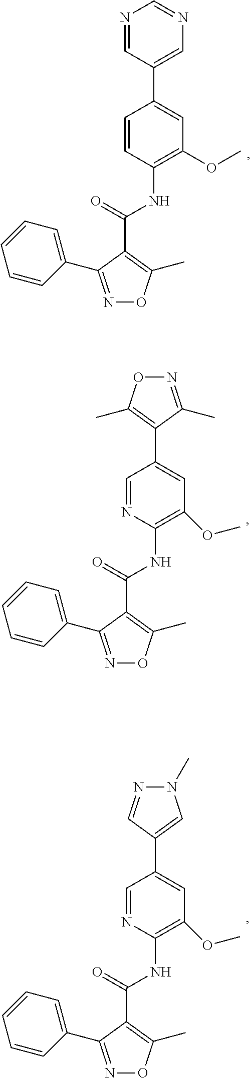 [SMILES: COc1cc(-c2cncnc2)ccc1NC(=O)c1c(-c2ccccc2)noc1C ; COc1cc(-c2c(C)noc2C)cnc1NC(=O)c1c(-c2ccccc2)noc1C ; COc1cc(-c2cnn(C)c2)cnc1NC(=O)c1c(-c2ccccc2)noc1C]

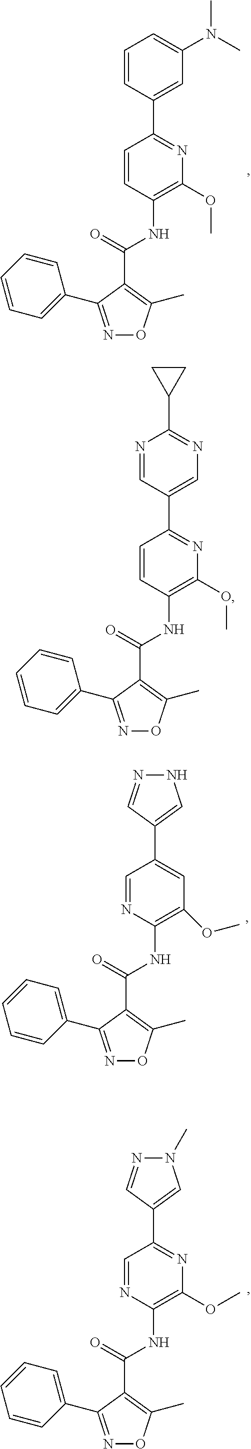

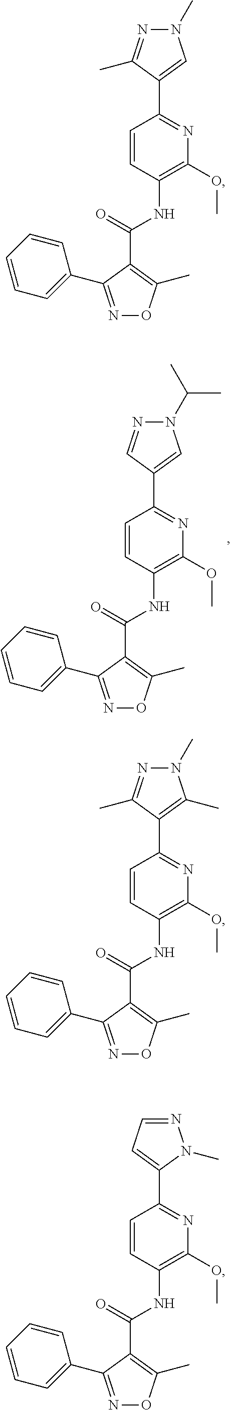 [SMILES: COc1nc(-c2cn(C)nc2C)ccc1NC(=O)c1c(-c2ccccc2)noc1C ; COc1nc(-c2cnn(C(C)C)c2)ccc1NC(=O)c1c(-c2ccccc2)noc1C ; COc1nc(-c2c(C)nn(C)c2C)ccc1NC(=O)c1c(-c2ccccc2)noc1C ; COc1nc(-c2ccnn2C)ccc1NC(=O)c1c(-c2ccccc2)noc1C]

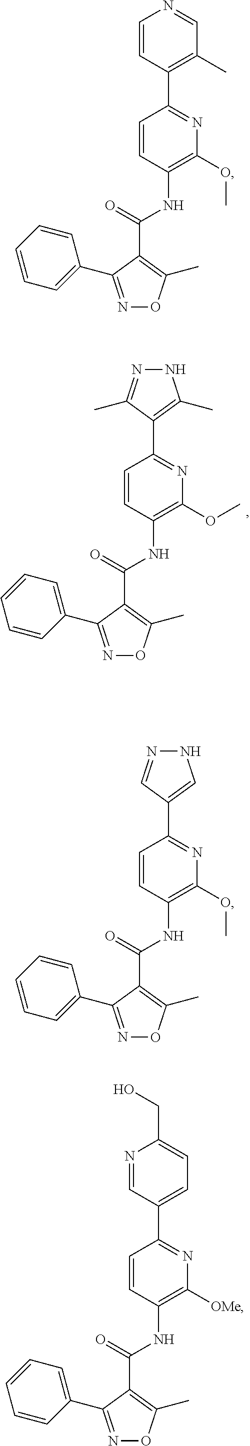 [SMILES: COc1nc(-c2ccncc2C)ccc1NC(=O)c1c(-c2ccccc2)noc1C ; COc1nc(-c2c(C)n[nH]c2C)ccc1NC(=O)c1c(-c2ccccc2)noc1C ; COc1nc(-c2cn[nH]c2)ccc1NC(=O)c1c(-c2ccccc2)noc1C ; COc1nc(-c2ccc(CO)nc2)ccc1NC(=O)c1c(-c2ccccc2)noc1C]

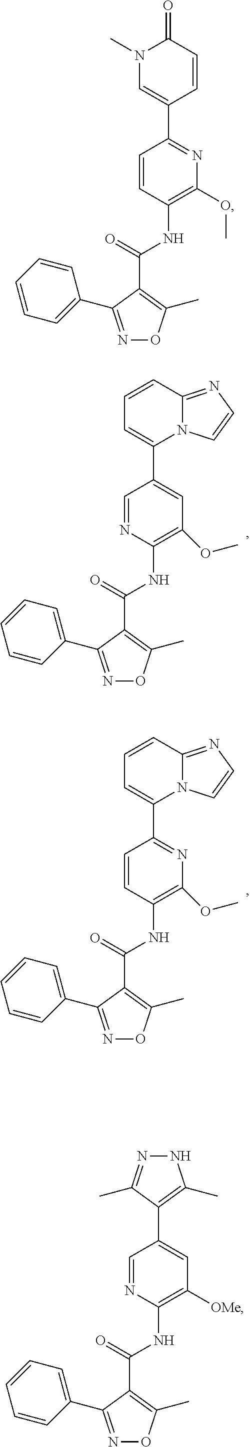

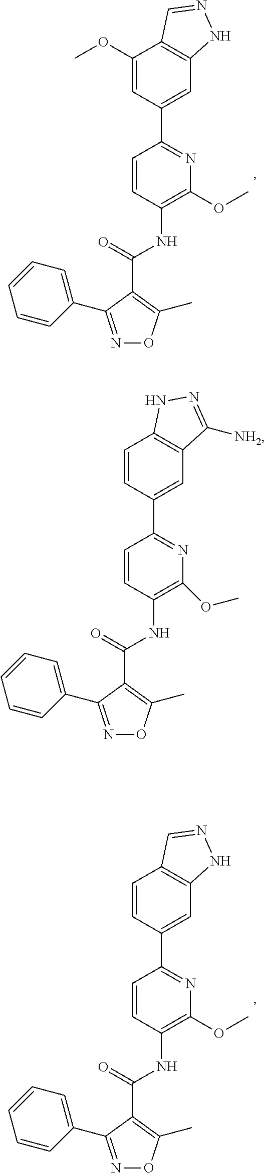 [SMILES: COc1nc(-c2cc(OC)c3cn[nH]c3c2)ccc1NC(=O)c1c(-c2ccccc2)noc1C ; COc1nc(-c2ccc3[nH]nc(N)c3c2)ccc1NC(=O)c1c(-c2ccccc2)noc1C ; COc1nc(-c2ccc3cn[nH]c3c2)ccc1NC(=O)c1c(-c2ccccc2)noc1C]

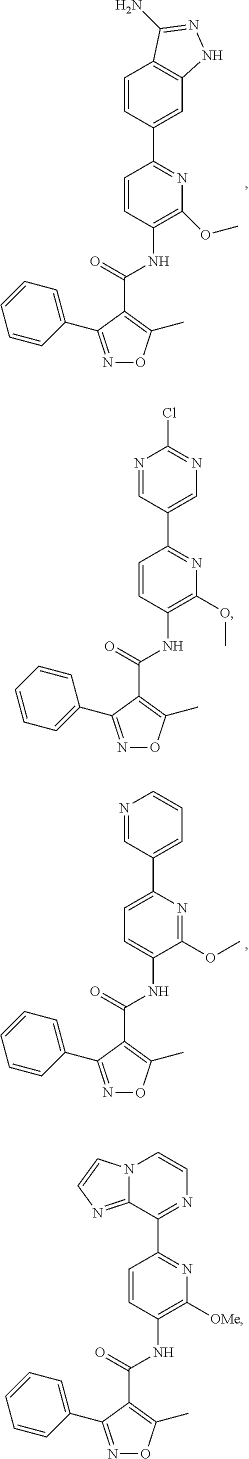

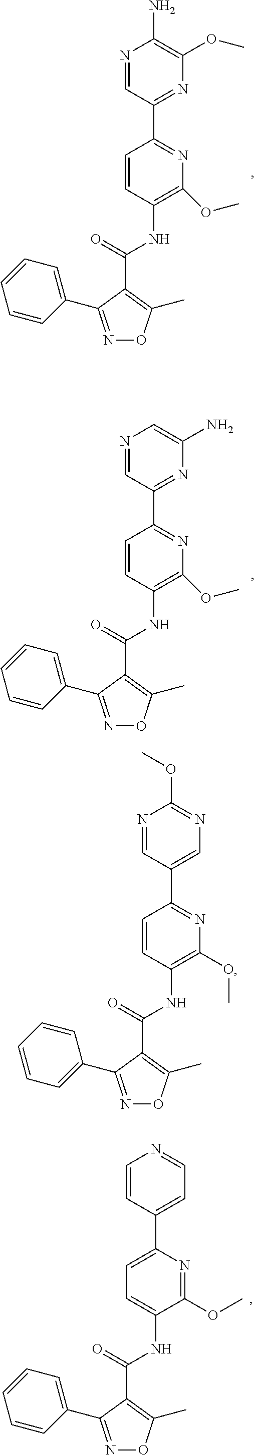

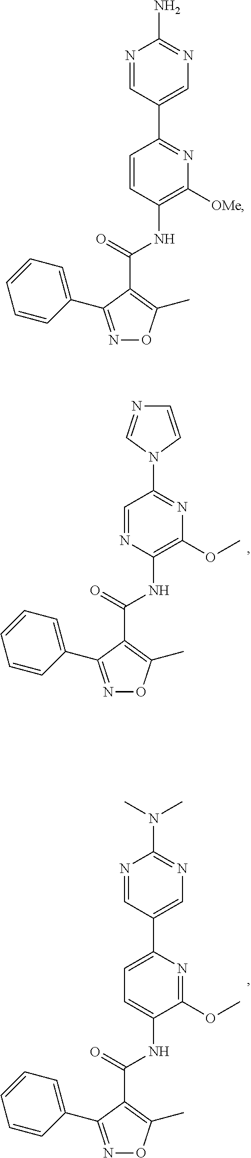 [SMILES: COc1nc(-c2cnc(N)nc2)ccc1NC(=O)c1c(-c2ccccc2)noc1C ; COc1nc(-n2ccnc2)cnc1NC(=O)c1c(-c2ccccc2)noc1C ; COc1nc(-c2cnc(N(C)C)nc2)ccc1NC(=O)c1c(-c2ccccc2)noc1C]

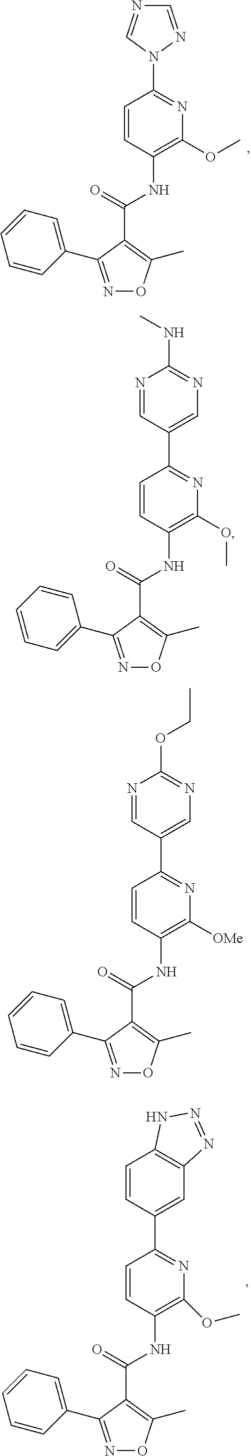

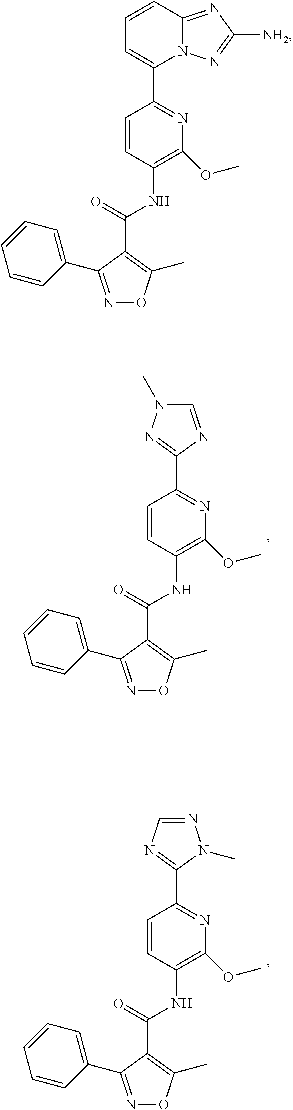 [SMILES: COc1nc(-c2cccc3nc(N)nn23)ccc1NC(=O)c1c(-c2ccccc2)noc1C ; COc1nc(-c2ncn(C)n2)ccc1NC(=O)c1c(-c2ccccc2)noc1C ; COc1nc(-c2ncnn2C)ccc1NC(=O)c1c(-c2ccccc2)noc1C]

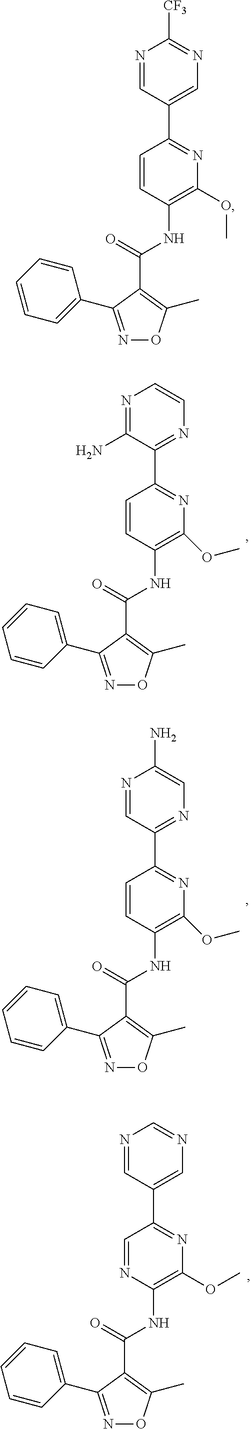

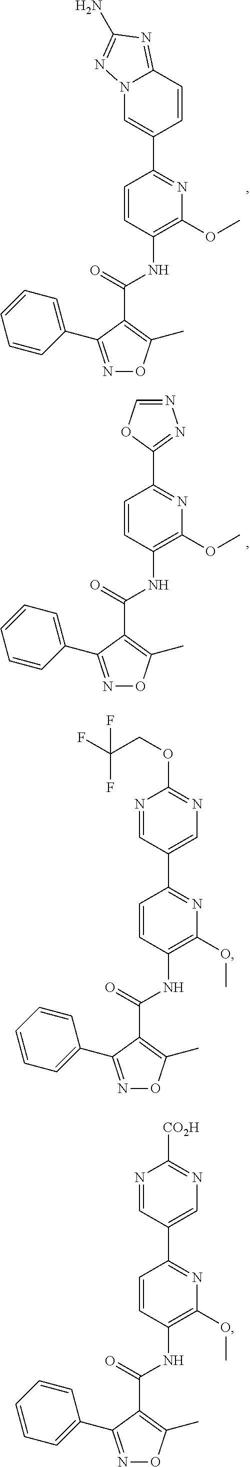

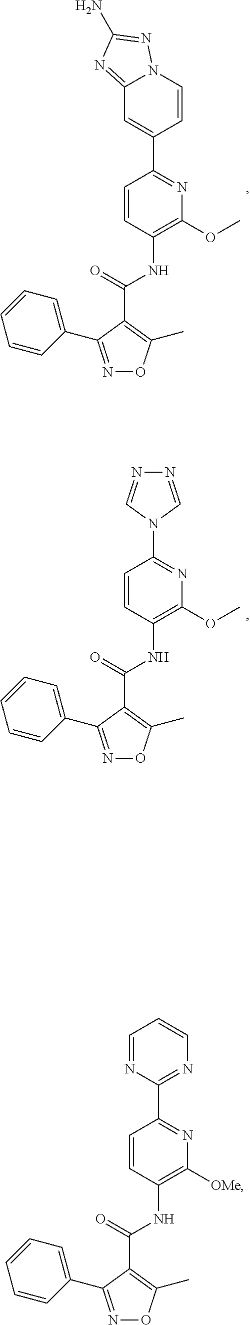 [SMILES: COc1nc(-c2ccn3nc(N)nc3c2)ccc1NC(=O)c1c(-c2ccccc2)noc1C ; COc1nc(-n2cnnc2)ccc1NC(=O)c1c(-c2ccccc2)noc1C ; COc1nc(-c2ncccn2)ccc1NC(=O)c1c(-c2ccccc2)noc1C]

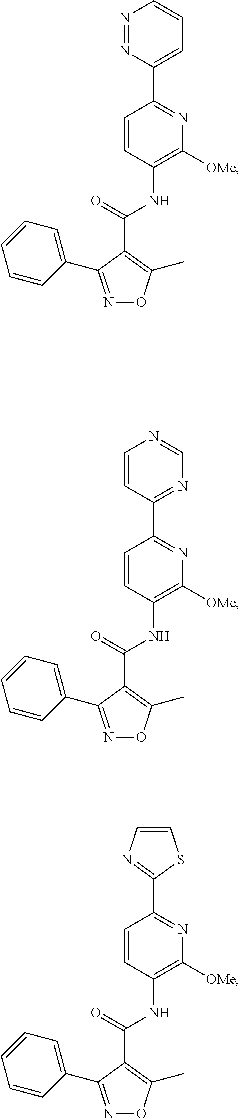 [SMILES: COc1nc(-c2cccnn2)ccc1NC(=O)c1c(-c2ccccc2)noc1C ; COc1nc(-c2ccncn2)ccc1NC(=O)c1c(-c2ccccc2)noc1C ; COc1nc(-c2nccs2)ccc1NC(=O)c1c(-c2ccccc2)noc1C]

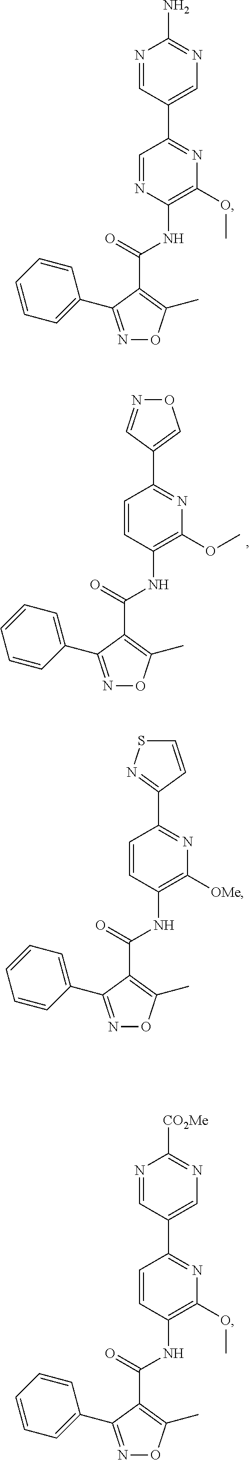 [SMILES: COc1nc(-c2cnc(N)nc2)cnc1NC(=O)c1c(-c2ccccc2)noc1C ; COc1nc(-c2cnoc2)ccc1NC(=O)c1c(-c2ccccc2)noc1C ; COc1nc(-c2ccsn2)ccc1NC(=O)c1c(-c2ccccc2)noc1C ; COC(=O)c1ncc(-c2ccc(NC(=O)c3c(-c4ccccc4)noc3C)c(OC)n2)cn1]

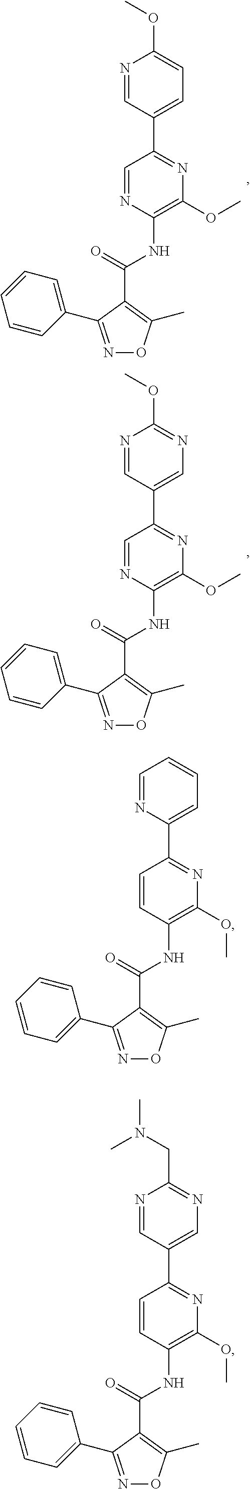

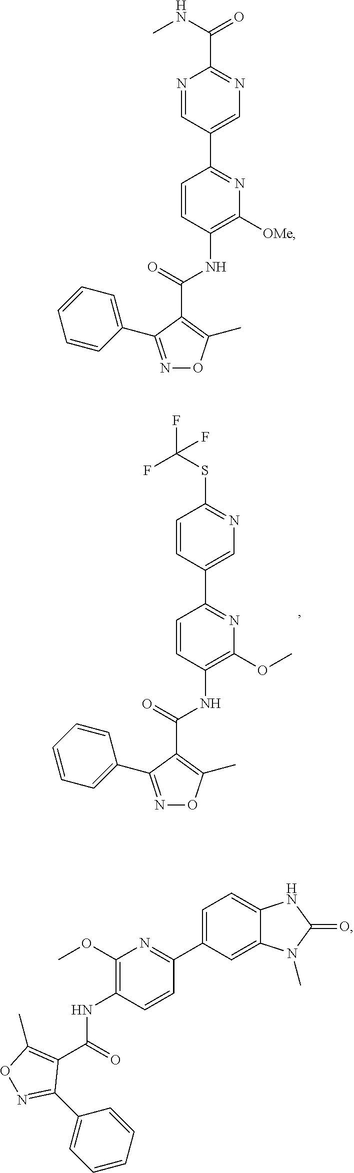 [SMILES: CNC(=O)c1ncc(-c2ccc(NC(=O)c3c(-c4ccccc4)noc3C)c(OC)n2)cn1 ; COc1nc(-c2ccc(SC(F)(F)F)nc2)ccc1NC(=O)c1c(-c2ccccc2)noc1C ; COc1nc(-c2ccc3[nH]c(=O)n(C)c3c2)ccc1NC(=O)c1c(-c2ccccc2)noc1C]

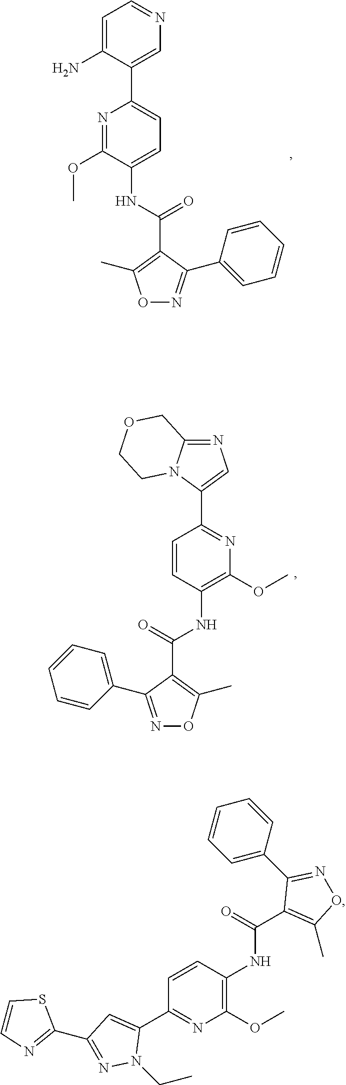 [SMILES: COc1nc(-c2cnccc2N)ccc1NC(=O)c1c(-c2ccccc2)noc1C ; COc1nc(-c2cnc3n2CCOC3)ccc1NC(=O)c1c(-c2ccccc2)noc1C ; CCn1nc(-c2nccs2)cc1-c1ccc(NC(=O)c2c(-c3ccccc3)noc2C)c(OC)n1]

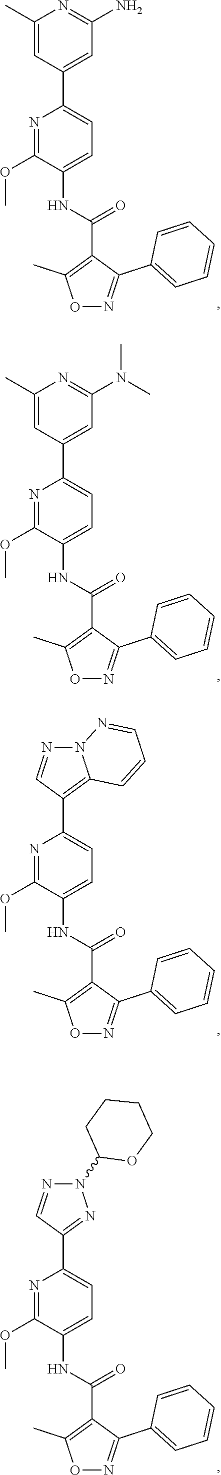

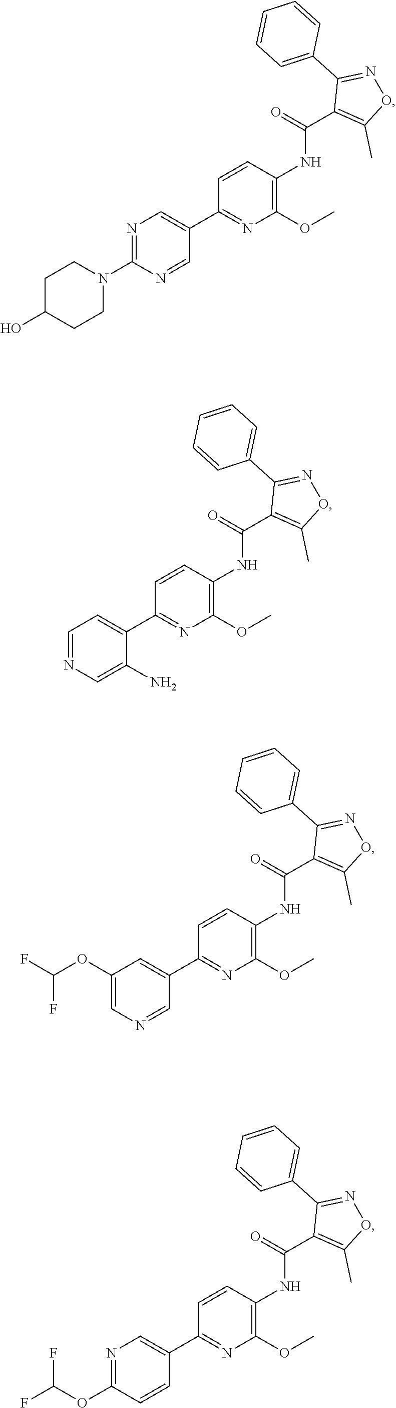 [SMILES: COc1nc(-c2cnc(N3CCC(O)CC3)nc2)ccc1NC(=O)c1c(-c2ccccc2)noc1C ; COc1nc(-c2ccncc2N)ccc1NC(=O)c1c(-c2ccccc2)noc1C ; COc1nc(-c2cncc(OC(F)F)c2)ccc1NC(=O)c1c(-c2ccccc2)noc1C ; COc1nc(-c2ccc(OC(F)F)nc2)ccc1NC(=O)c1c(-c2ccccc2)noc1C]

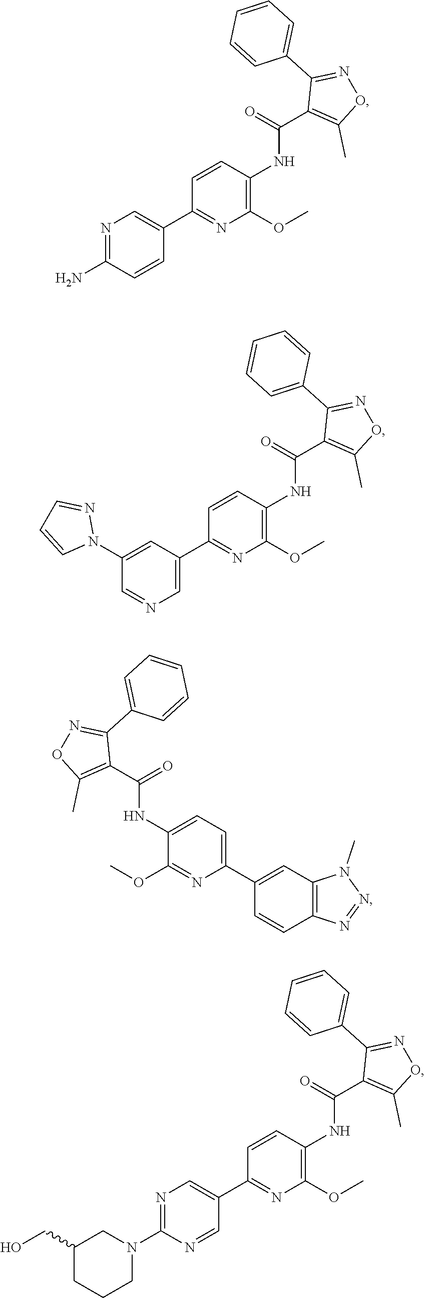 [SMILES: COc1nc(-c2ccc(N)nc2)ccc1NC(=O)c1c(-c2ccccc2)noc1C ; COc1nc(-c2cncc(-n3cccn3)c2)ccc1NC(=O)c1c(-c2ccccc2)noc1C ; COc1nc(-c2ccc3nnn(C)c3c2)ccc1NC(=O)c1c(-c2ccccc2)noc1C ; COc1nc(-c2cnc(N3CCCC(CO)C3)nc2)ccc1NC(=O)c1c(-c2ccccc2)noc1C]

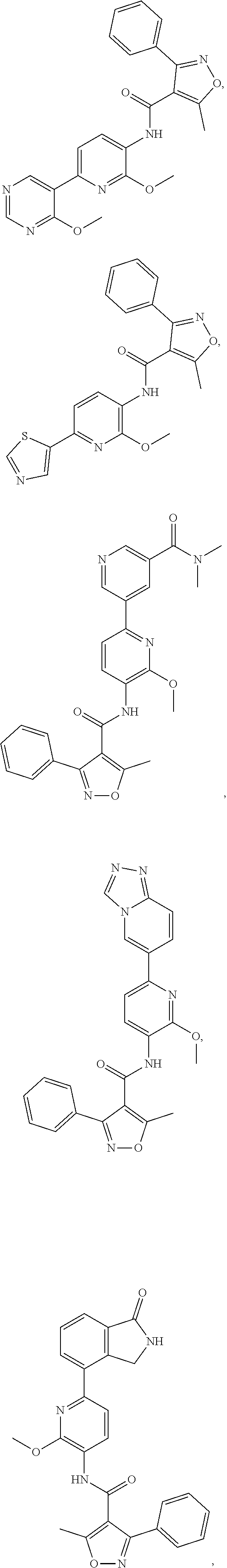

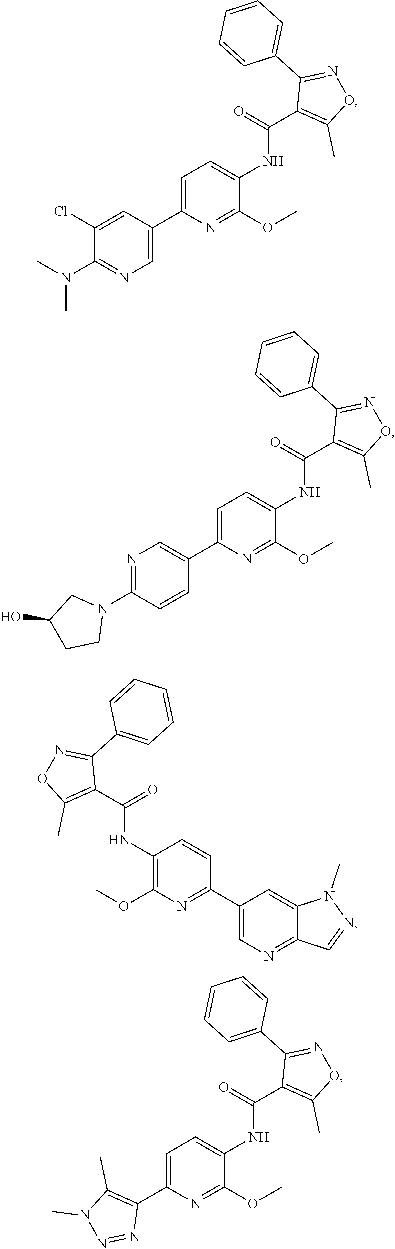

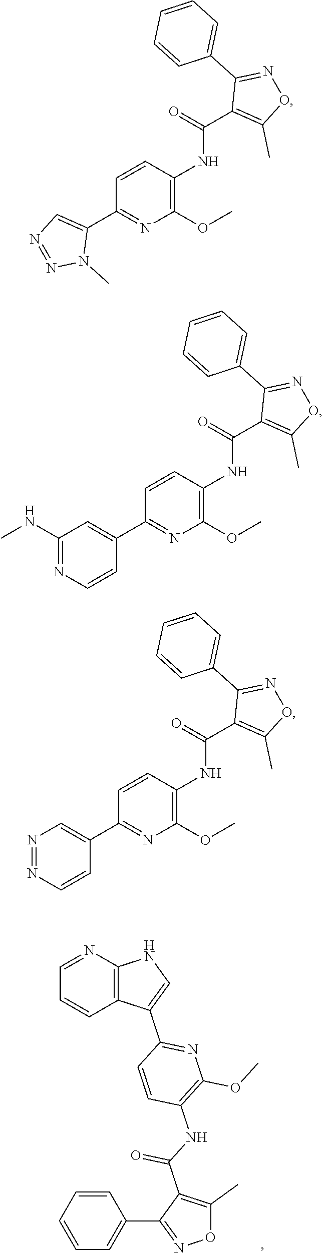 [SMILES: COc1nc(-c2cnnn2C)ccc1NC(=O)c1c(-c2ccccc2)noc1C ; CNc1cc(-c2ccc(NC(=O)c3c(-c4ccccc4)noc3C)c(OC)n2)ccn1 ; COc1nc(-c2ccnnc2)ccc1NC(=O)c1c(-c2ccccc2)noc1C ; COc1nc(-c2c[nH]c3ncccc23)ccc1NC(=O)c1c(-c2ccccc2)noc1C]

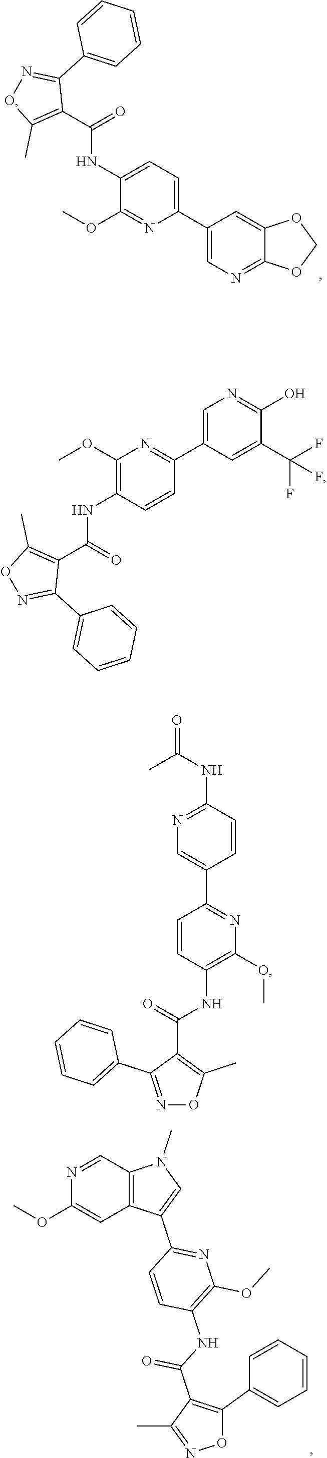

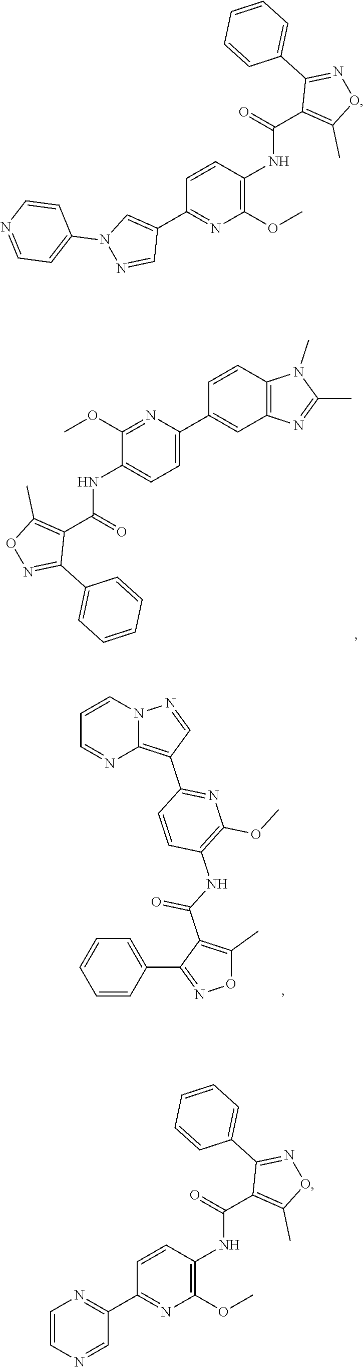 [SMILES: COc1nc(-c2cnn(-c3ccncc3)c2)ccc1NC(=O)c1c(-c2ccccc2)noc1C ; COc1nc(-c2ccc3c(c2)nc(C)n3C)ccc1NC(=O)c1c(-c2ccccc2)noc1C ; COc1nc(-c2cnn3cccnc23)ccc1NC(=O)c1c(-c2ccccc2)noc1C ; COc1nc(-c2cnccn2)ccc1NC(=O)c1c(-c2ccccc2)noc1C]

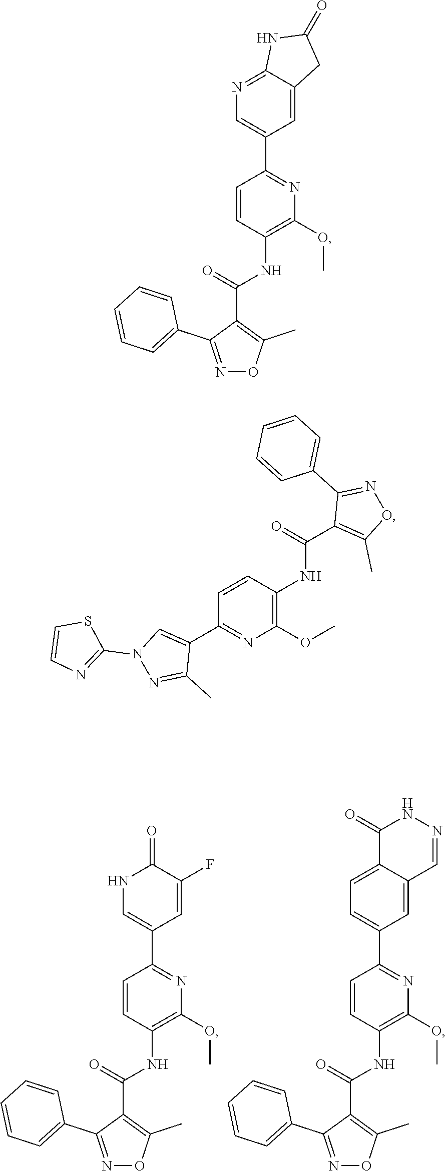

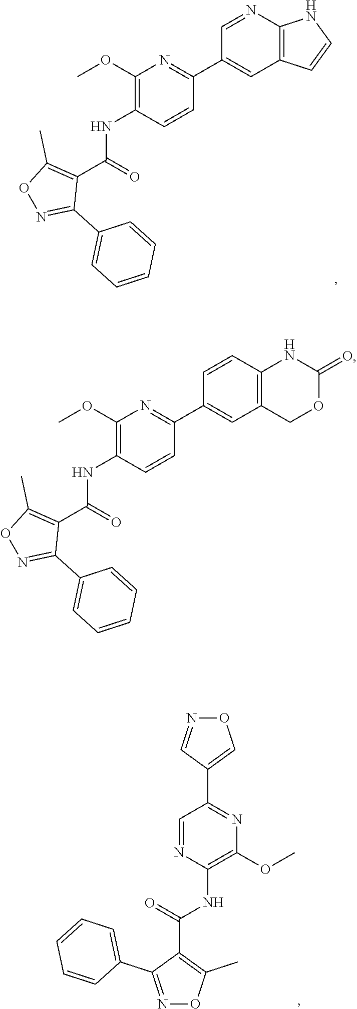 [SMILES: COc1nc(-c2cnc3[nH]ccc3c2)ccc1NC(=O)c1c(-c2ccccc2)noc1C ; COc1nc(-c2ccc3c(c2)COC(=O)N3)ccc1NC(=O)c1c(-c2ccccc2)noc1C ; COc1nc(-c2cnoc2)cnc1NC(=O)c1c(-c2ccccc2)noc1C]

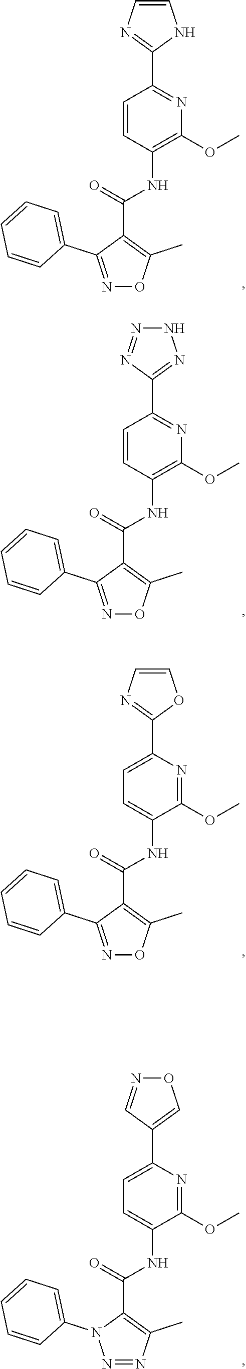

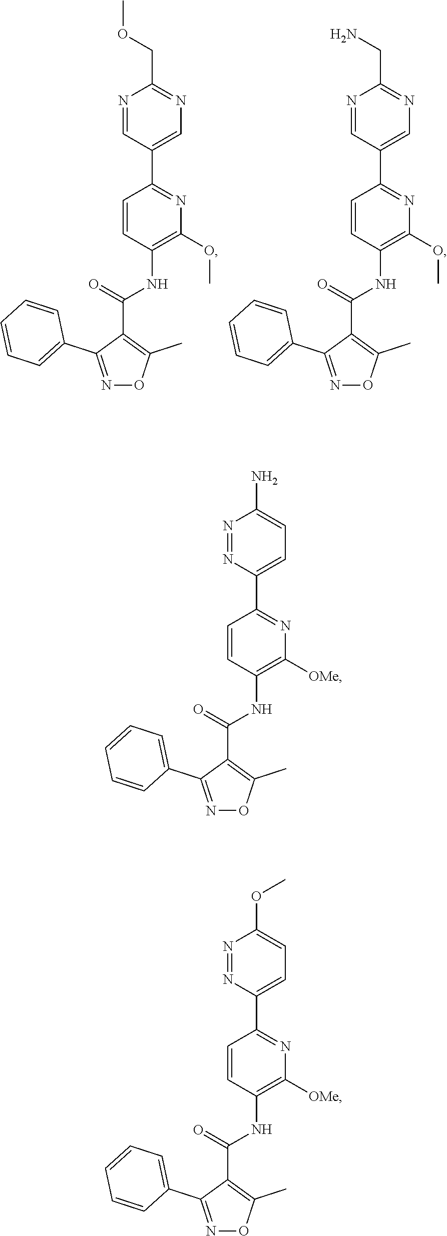

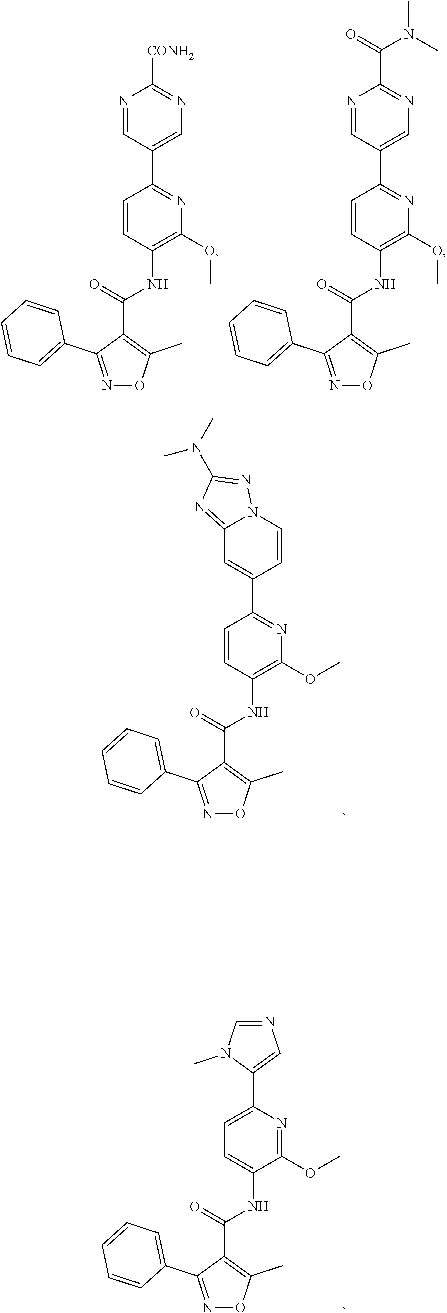

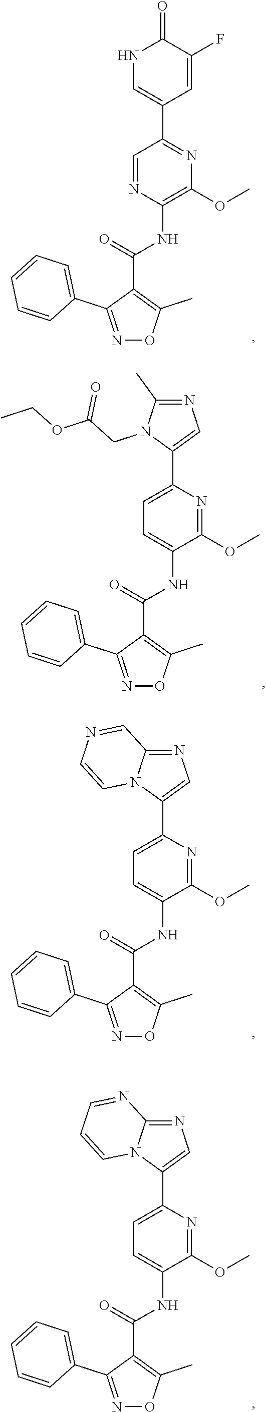

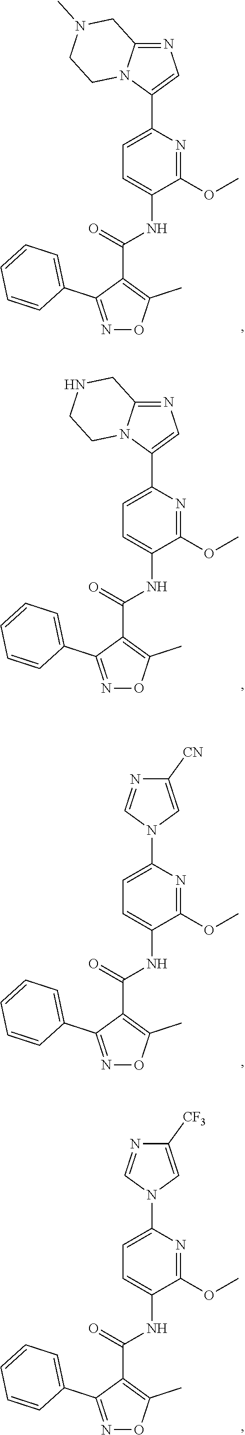 [SMILES: COc1nc(-c2cnc3n2CCN(C)C3)ccc1NC(=O)c1c(-c2ccccc2)noc1C ; COc1nc(-c2cnc3n2CCNC3)ccc1NC(=O)c1c(-c2ccccc2)noc1C ; COc1nc(-n2cnc(C#N)c2)ccc1NC(=O)c1c(-c2ccccc2)noc1C ; COc1nc(-n2cnc(C(F)(F)F)c2)ccc1NC(=O)c1c(-c2ccccc2)noc1C]

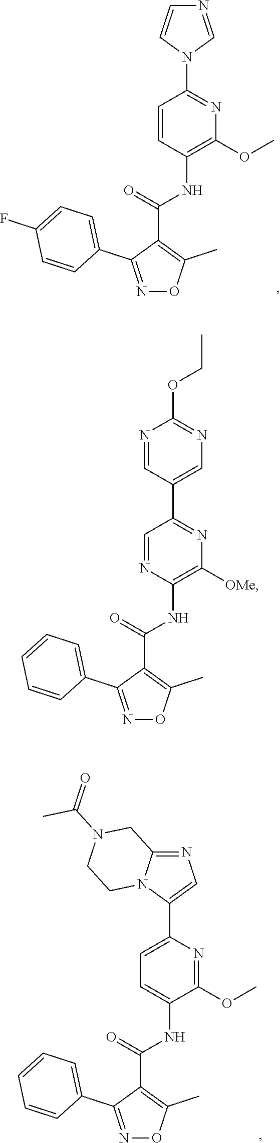 [SMILES: COc1nc(-n2ccnc2)ccc1NC(=O)c1c(-c2ccc(F)cc2)noc1C ; CCOc1ncc(-c2cnc(NC(=O)c3c(-c4ccccc4)noc3C)c(OC)n2)cn1 ; COc1nc(-c2cnc3n2CCN(C(C)=O)C3)ccc1NC(=O)c1c(-c2ccccc2)noc1C]

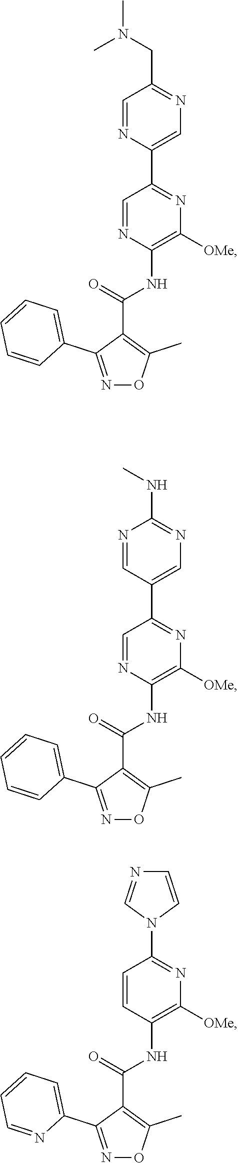 [SMILES: COc1nc(-c2cnc(CN(C)C)cn2)cnc1NC(=O)c1c(-c2ccccc2)noc1C ; CNc1ncc(-c2cnc(NC(=O)c3c(-c4ccccc4)noc3C)c(OC)n2)cn1 ; COc1nc(-n2ccnc2)ccc1NC(=O)c1c(-c2ccccn2)noc1C]

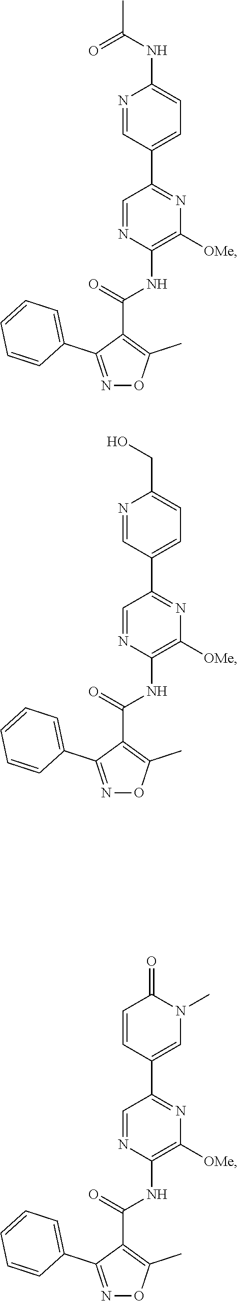 [SMILES: COc1nc(-c2ccc(NC(C)=O)nc2)cnc1NC(=O)c1c(-c2ccccc2)noc1C ; COc1nc(-c2ccc(CO)nc2)cnc1NC(=O)c1c(-c2ccccc2)noc1C ; COc1nc(-c2ccc(=O)n(C)c2)cnc1NC(=O)c1c(-c2ccccc2)noc1C]

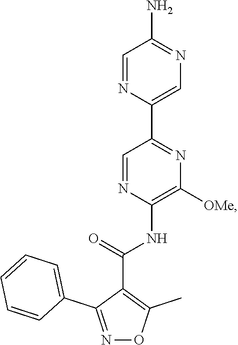 [SMILES: COc1nc(-c2cnc(N)cn2)cnc1NC(=O)c1c(-c2ccccc2)noc1C]

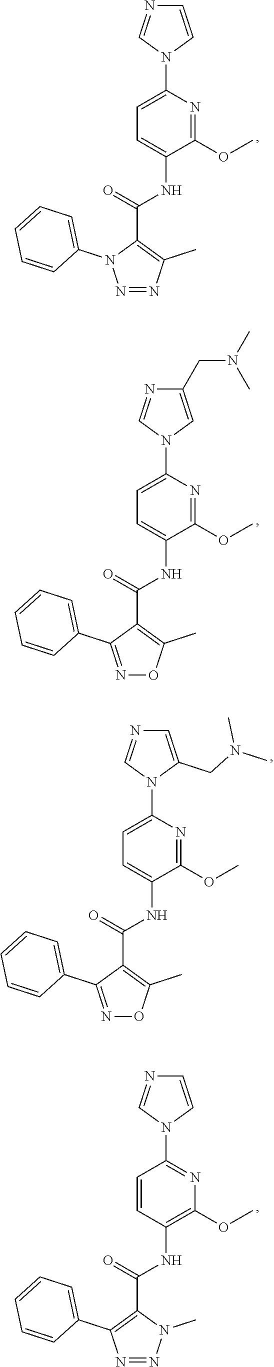 [SMILES: COc1nc(-n2ccnc2)ccc1NC(=O)c1c(C)nnn1-c1ccccc1 ; COc1nc(-n2cnc(CN(C)C)c2)ccc1NC(=O)c1c(-c2ccccc2)noc1C ; COc1nc(-n2cncc2CN(C)C)ccc1NC(=O)c1c(-c2ccccc2)noc1C ; COc1nc(-n2ccnc2)ccc1NC(=O)c1c(-c2ccccc2)nnn1C]

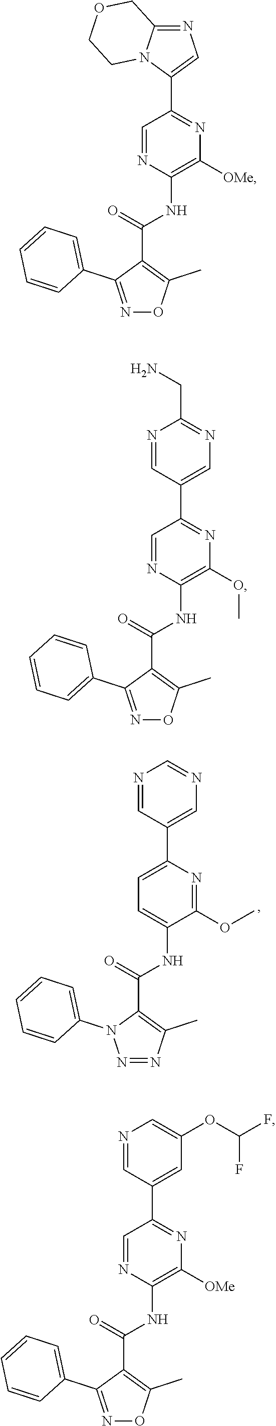

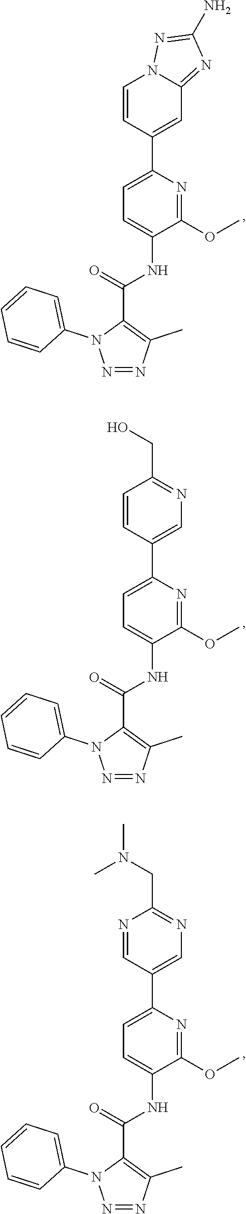 [SMILES: COc1nc(-c2ccn3nc(N)nc3c2)ccc1NC(=O)c1c(C)nnn1-c1ccccc1 ; COc1nc(-c2ccc(CO)nc2)ccc1NC(=O)c1c(C)nnn1-c1ccccc1 ; COc1nc(-c2cnc(CN(C)C)nc2)ccc1NC(=O)c1c(C)nnn1-c1ccccc1]

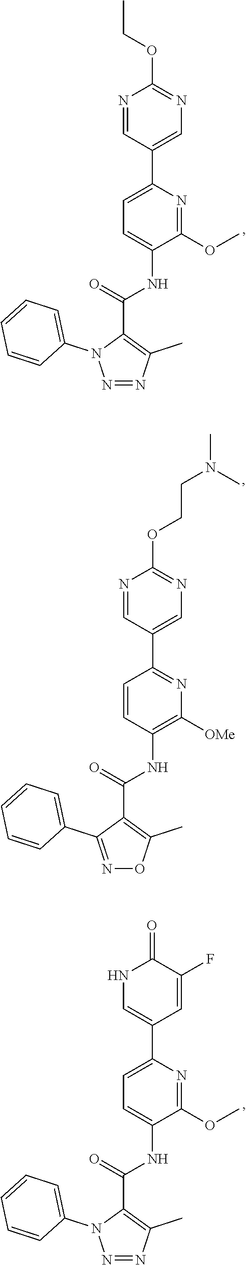 [SMILES: CCOc1ncc(-c2ccc(NC(=O)c3c(C)nnn3-c3ccccc3)c(OC)n2)cn1 ; COc1nc(-c2cnc(OCCN(C)C)nc2)ccc1NC(=O)c1c(-c2ccccc2)noc1C ; COc1nc(-c2c[nH]c(=O)c(F)c2)ccc1NC(=O)c1c(C)nnn1-c1ccccc1]

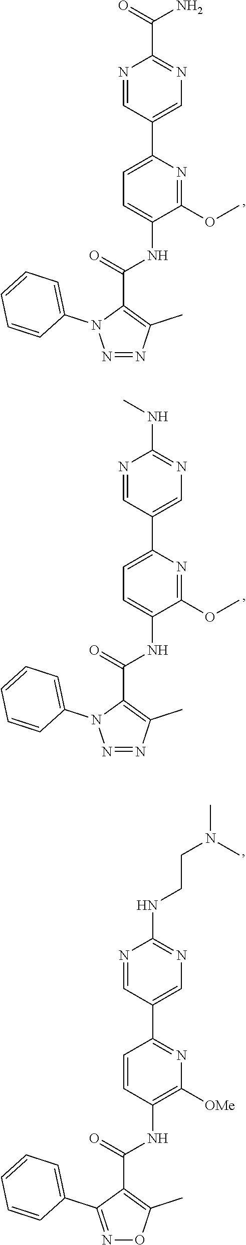

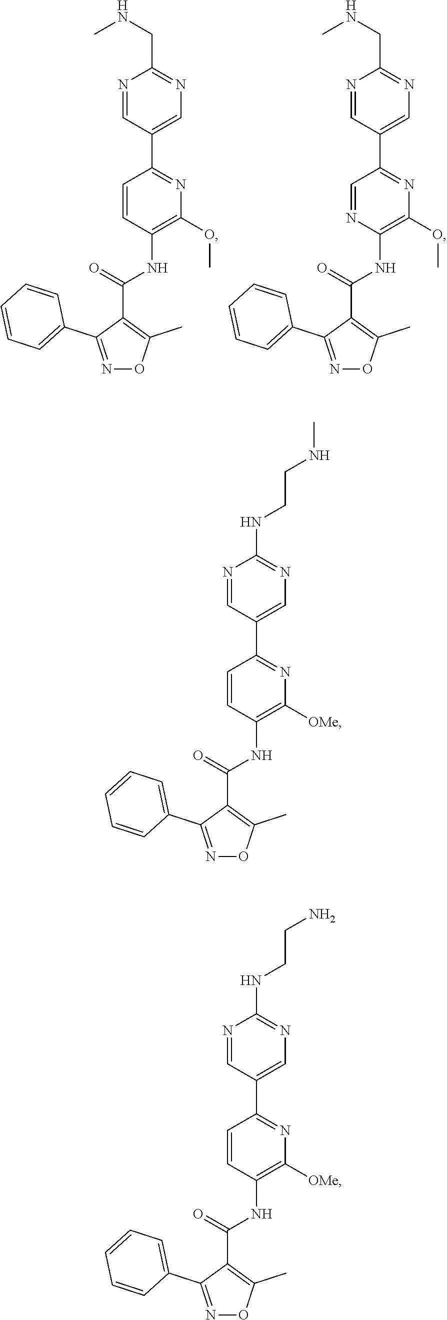

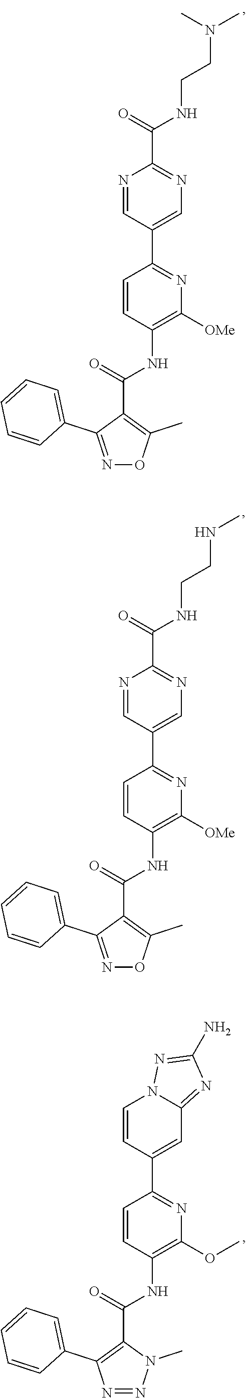 [SMILES: COc1nc(-c2cnc(C(=O)NCCN(C)C)nc2)ccc1NC(=O)c1c(-c2ccccc2)noc1C ; CNCCNC(=O)c1ncc(-c2ccc(NC(=O)c3c(-c4ccccc4)noc3C)c(OC)n2)cn1 ; COc1nc(-c2ccn3nc(N)nc3c2)ccc1NC(=O)c1c(-c2ccccc2)nnn1C]

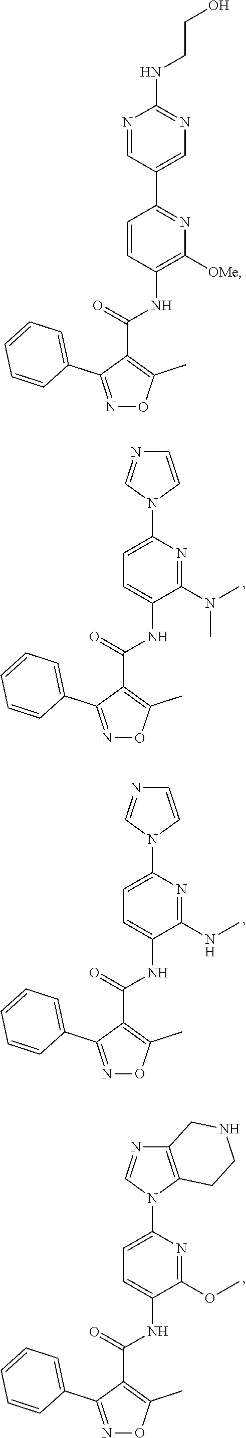

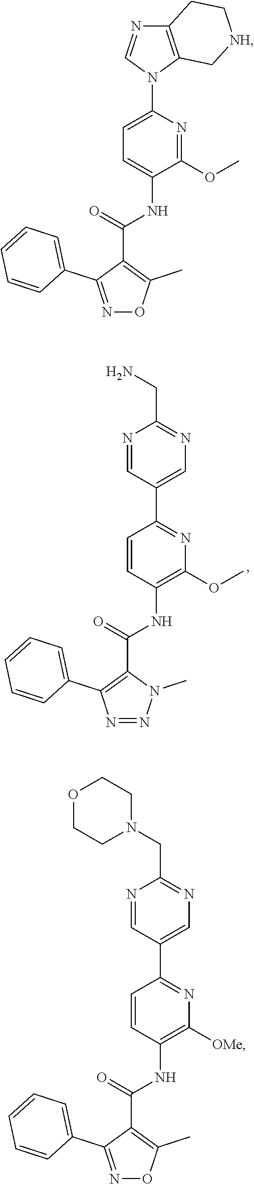 [SMILES: COc1nc(-n2cnc3c2CNCC3)ccc1NC(=O)c1c(-c2ccccc2)noc1C ; COc1nc(-c2cnc(CN)nc2)ccc1NC(=O)c1c(-c2ccccc2)nnn1C ; COc1nc(-c2cnc(CN3CCOCC3)nc2)ccc1NC(=O)c1c(-c2ccccc2)noc1C]

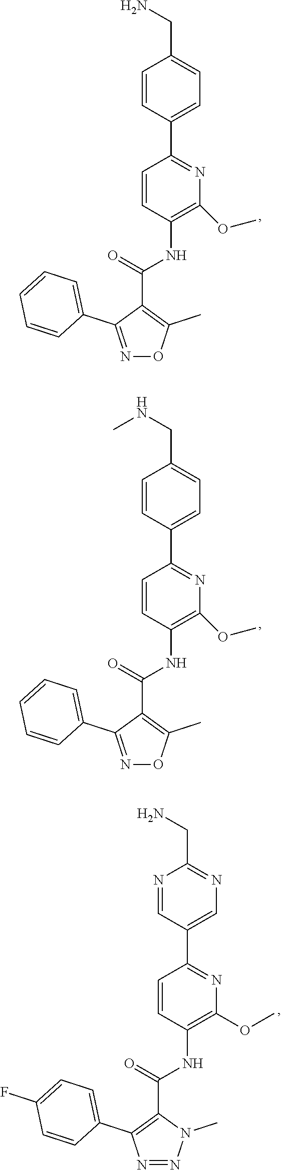 [SMILES: COc1nc(-c2ccc(CN)cc2)ccc1NC(=O)c1c(-c2ccccc2)noc1C ; CNCc1ccc(-c2ccc(NC(=O)c3c(-c4ccccc4)noc3C)c(OC)n2)cc1 ; COc1nc(-c2cnc(CN)nc2)ccc1NC(=O)c1c(-c2ccc(F)cc2)nnn1C]

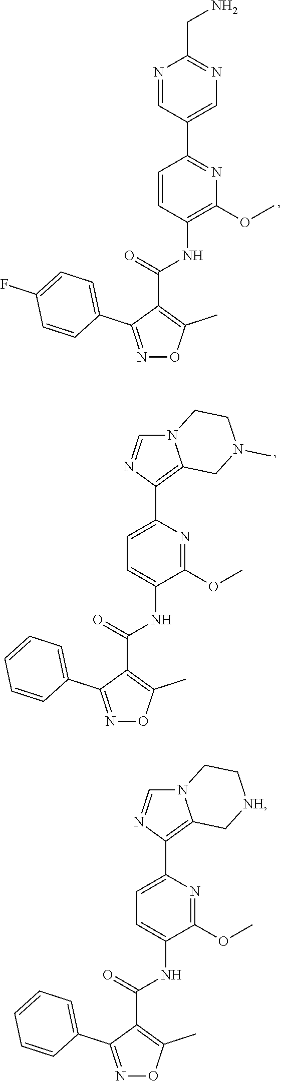 [SMILES: COc1nc(-c2cnc(CN)nc2)ccc1NC(=O)c1c(-c2ccc(F)cc2)noc1C ; COc1nc(-c2ncn3c2CN(C)CC3)ccc1NC(=O)c1c(-c2ccccc2)noc1C ; COc1nc(-c2ncn3c2CNCC3)ccc1NC(=O)c1c(-c2ccccc2)noc1C]

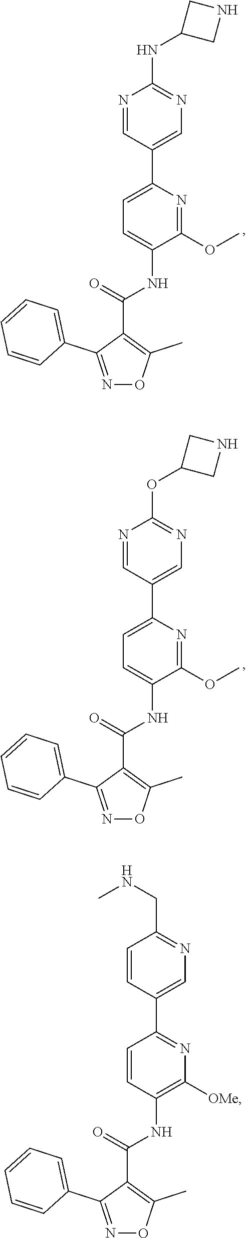 [SMILES: COc1nc(-c2cnc(NC3CNC3)nc2)ccc1NC(=O)c1c(-c2ccccc2)noc1C ; COc1nc(-c2cnc(OC3CNC3)nc2)ccc1NC(=O)c1c(-c2ccccc2)noc1C ; CNCc1ccc(-c2ccc(NC(=O)c3c(-c4ccccc4)noc3C)c(OC)n2)cn1]

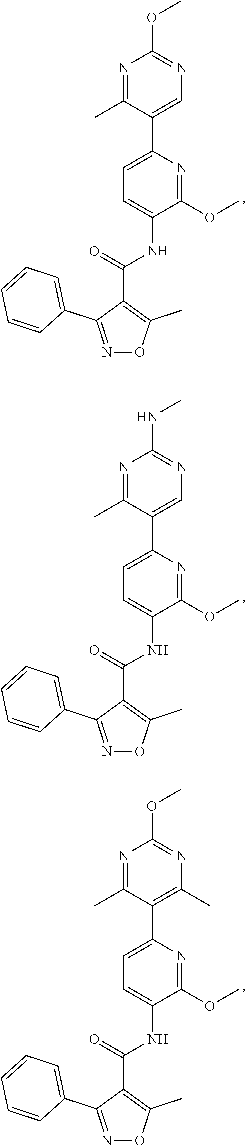 [SMILES: COc1ncc(-c2ccc(NC(=O)c3c(-c4ccccc4)noc3C)c(OC)n2)c(C)n1 ; CNc1ncc(-c2ccc(NC(=O)c3c(-c4ccccc4)noc3C)c(OC)n2)c(C)n1 ; COc1nc(C)c(-c2ccc(NC(=O)c3c(-c4ccccc4)noc3C)c(OC)n2)c(C)n1]

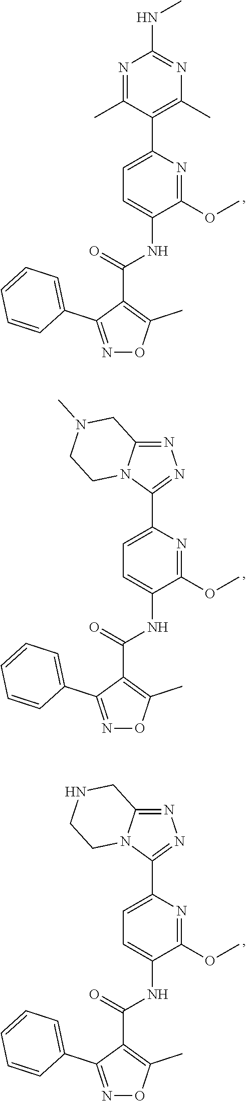 [SMILES: CNc1nc(C)c(-c2ccc(NC(=O)c3c(-c4ccccc4)noc3C)c(OC)n2)c(C)n1 ; COc1nc(-c2nnc3n2CCN(C)C3)ccc1NC(=O)c1c(-c2ccccc2)noc1C ; COc1nc(-c2nnc3n2CCNC3)ccc1NC(=O)c1c(-c2ccccc2)noc1C]

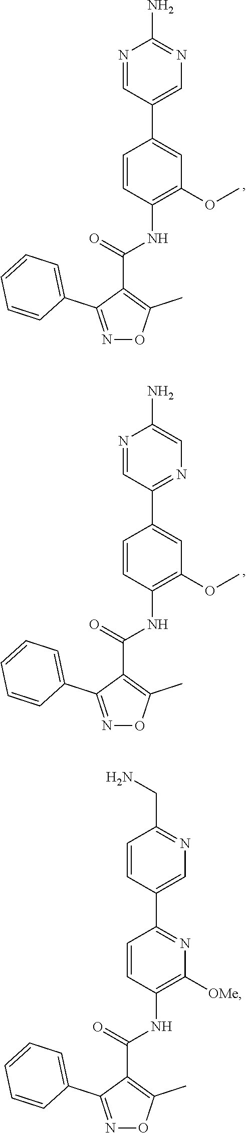 [SMILES: COc1cc(-c2cnc(N)nc2)ccc1NC(=O)c1c(-c2ccccc2)noc1C ; COc1cc(-c2cnc(N)cn2)ccc1NC(=O)c1c(-c2ccccc2)noc1C ; COc1nc(-c2ccc(CN)nc2)ccc1NC(=O)c1c(-c2ccccc2)noc1C]

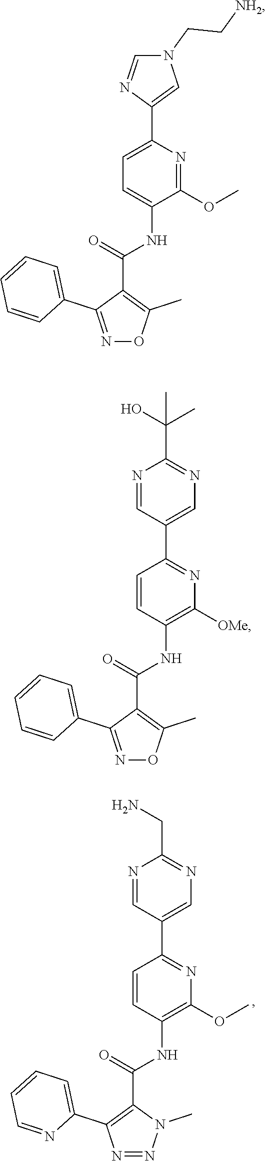

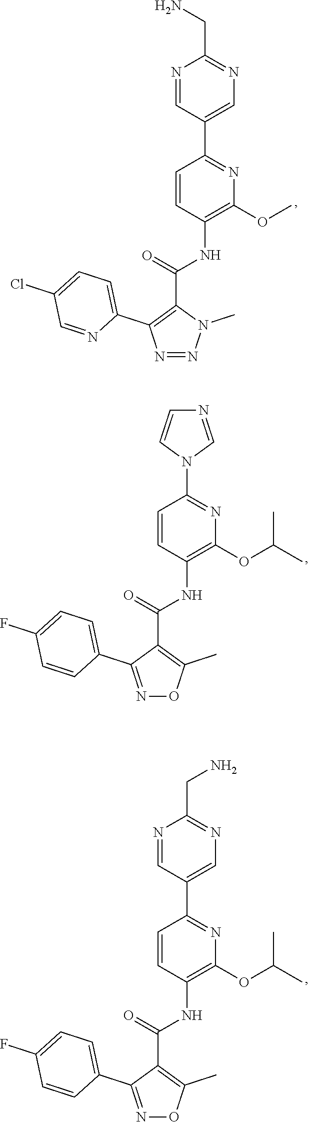 [SMILES: COc1nc(-c2cnc(CN)nc2)ccc1NC(=O)c1c(-c2ccc(Cl)cn2)nnn1C ; Cc1onc(-c2ccc(F)cc2)c1C(=O)Nc1ccc(-n2ccnc2)nc1OC(C)C ; Cc1onc(-c2ccc(F)cc2)c1C(=O)Nc1ccc(-c2cnc(CN)nc2)nc1OC(C)C]

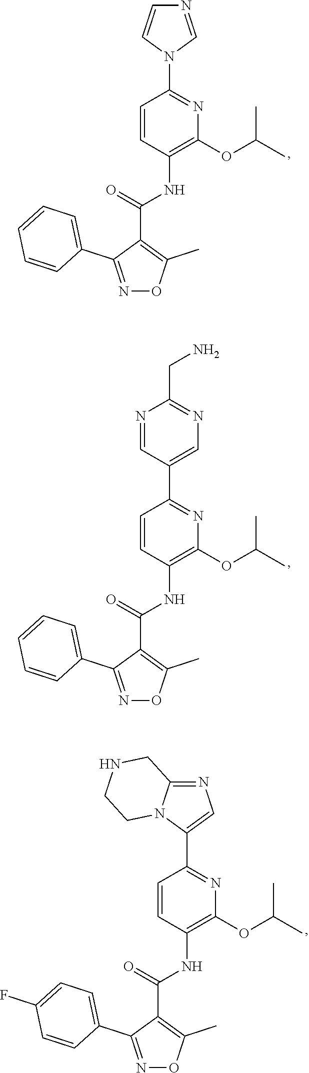 [SMILES: Cc1onc(-c2ccccc2)c1C(=O)Nc1ccc(-n2ccnc2)nc1OC(C)C ; Cc1onc(-c2ccccc2)c1C(=O)Nc1ccc(-c2cnc(CN)nc2)nc1OC(C)C ; Cc1onc(-c2ccc(F)cc2)c1C(=O)Nc1ccc(-c2cnc3n2CCNC3)nc1OC(C)C]

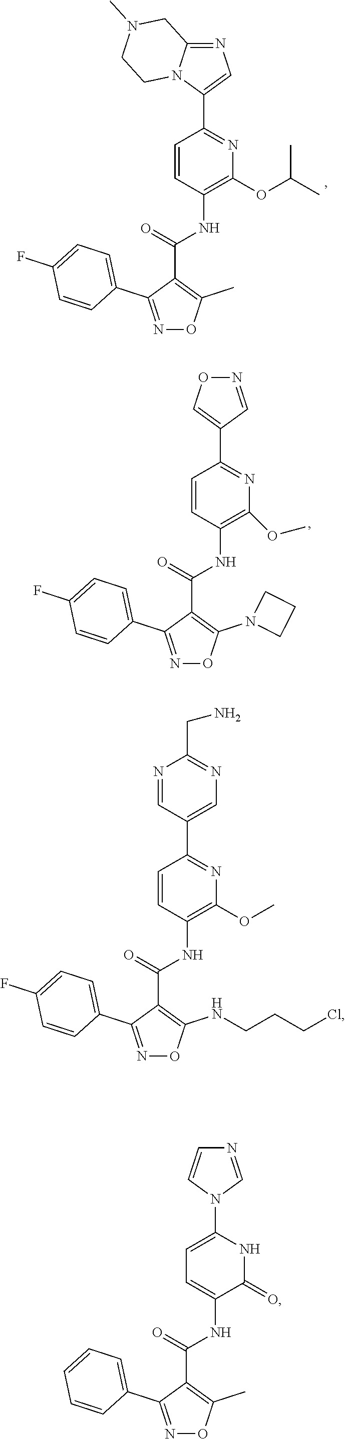 [SMILES: Cc1onc(-c2ccc(F)cc2)c1C(=O)Nc1ccc(-c2cnc3n2CCN(C)C3)nc1OC(C)C ; COc1nc(-c2cnoc2)ccc1NC(=O)c1c(-c2ccc(F)cc2)noc1N1CCC1 ; COc1nc(-c2cnc(CN)nc2)ccc1NC(=O)c1c(-c2ccc(F)cc2)noc1NCCCCl ; Cc1onc(-c2ccccc2)c1C(=O)Nc1ccc(-n2ccnc2)[nH]c1=O]

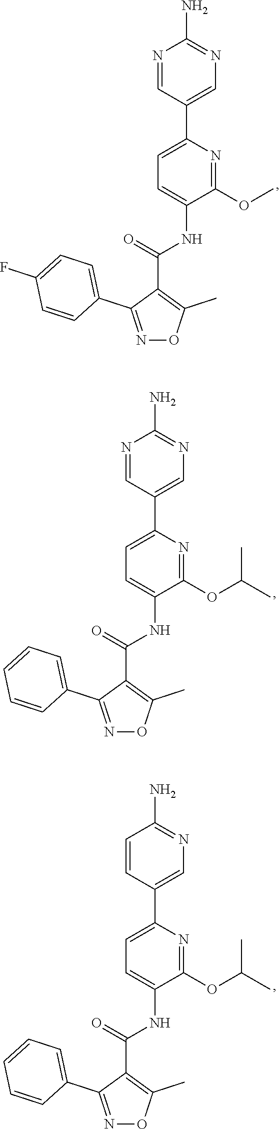 [SMILES: COc1nc(-c2cnc(N)nc2)ccc1NC(=O)c1c(-c2ccc(F)cc2)noc1C ; Cc1onc(-c2ccccc2)c1C(=O)Nc1ccc(-c2cnc(N)nc2)nc1OC(C)C ; Cc1onc(-c2ccccc2)c1C(=O)Nc1ccc(-c2ccc(N)nc2)nc1OC(C)C]

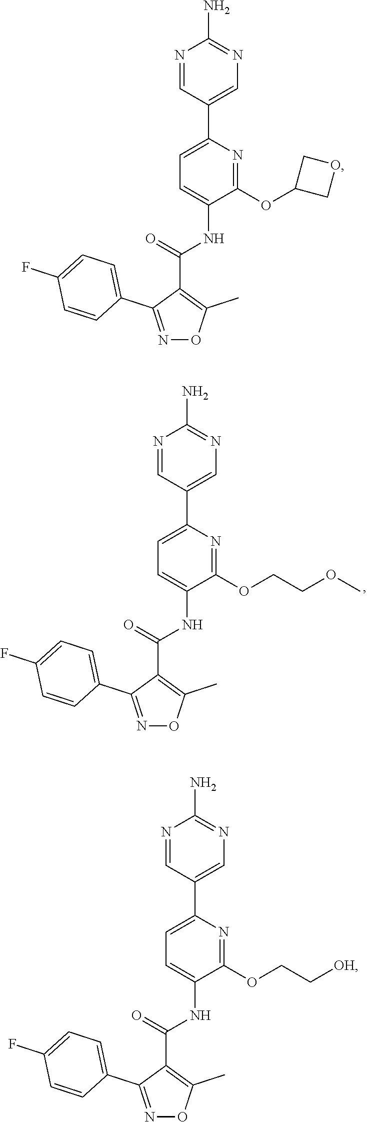 [SMILES: Cc1onc(-c2ccc(F)cc2)c1C(=O)Nc1ccc(-c2cnc(N)nc2)nc1OC1COC1 ; COCCOc1nc(-c2cnc(N)nc2)ccc1NC(=O)c1c(-c2ccc(F)cc2)noc1C ; Cc1onc(-c2ccc(F)cc2)c1C(=O)Nc1ccc(-c2cnc(N)nc2)nc1OCCO]

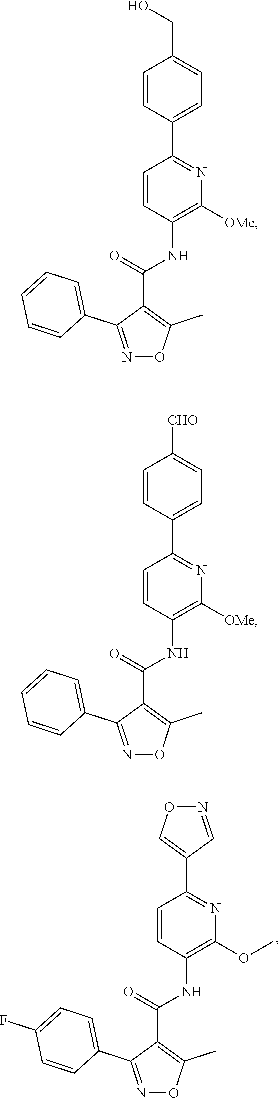 [SMILES: COc1nc(-c2ccc(CO)cc2)ccc1NC(=O)c1c(-c2ccccc2)noc1C ; COc1nc(-c2ccc(C=O)cc2)ccc1NC(=O)c1c(-c2ccccc2)noc1C ; COc1nc(-c2cnoc2)ccc1NC(=O)c1c(-c2ccc(F)cc2)noc1C]

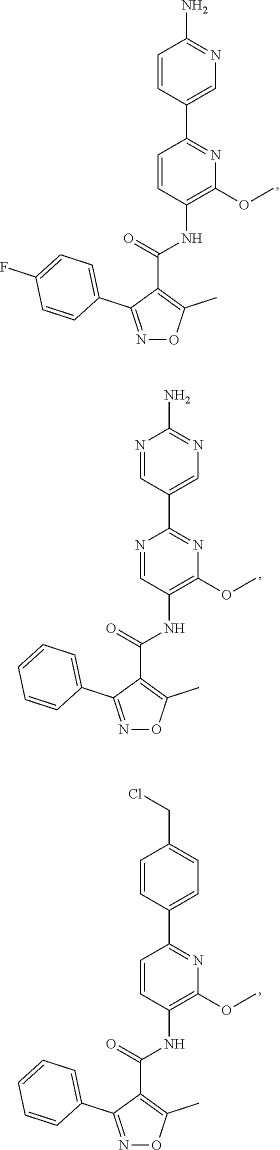 [SMILES: COc1nc(-c2ccc(N)nc2)ccc1NC(=O)c1c(-c2ccc(F)cc2)noc1C ; COc1nc(-c2cnc(N)nc2)ncc1NC(=O)c1c(-c2ccccc2)noc1C ; COc1nc(-c2ccc(CCl)cc2)ccc1NC(=O)c1c(-c2ccccc2)noc1C]

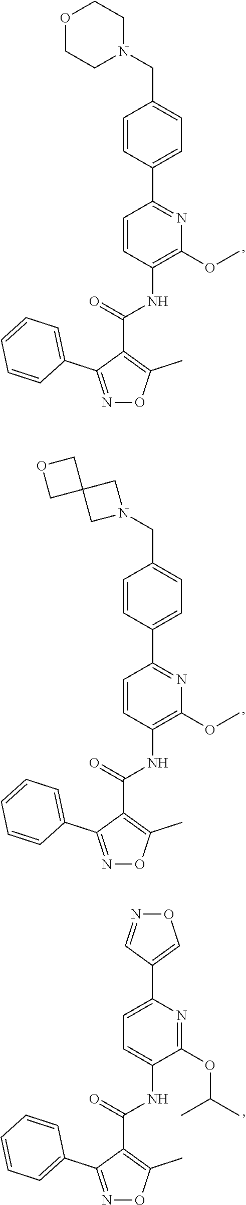 [SMILES: COc1nc(-c2ccc(CN3CCOCC3)cc2)ccc1NC(=O)c1c(-c2ccccc2)noc1C ; COc1nc(-c2ccc(CN3CC4(COC4)C3)cc2)ccc1NC(=O)c1c(-c2ccccc2)noc1C ; Cc1onc(-c2ccccc2)c1C(=O)Nc1ccc(-c2cnoc2)nc1OC(C)C]

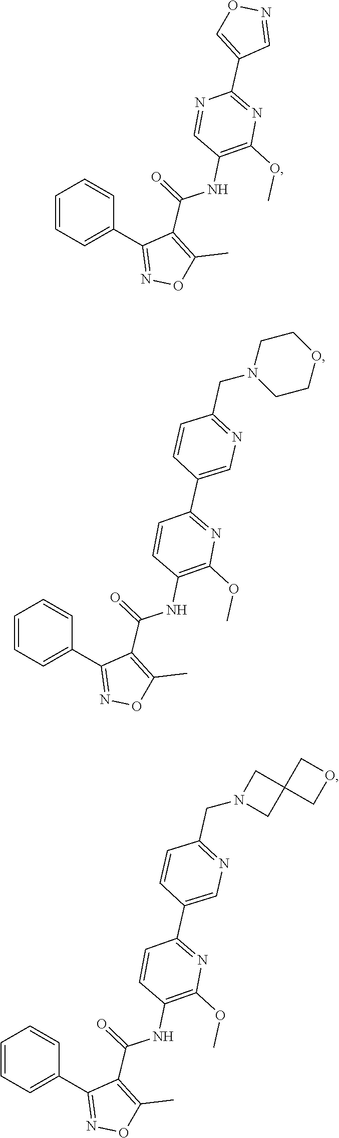 [SMILES: COc1nc(-c2cnoc2)ncc1NC(=O)c1c(-c2ccccc2)noc1C ; COc1nc(-c2ccc(CN3CCOCC3)nc2)ccc1NC(=O)c1c(-c2ccccc2)noc1C ; COc1nc(-c2ccc(CN3CC4(COC4)C3)nc2)ccc1NC(=O)c1c(-c2ccccc2)noc1C]

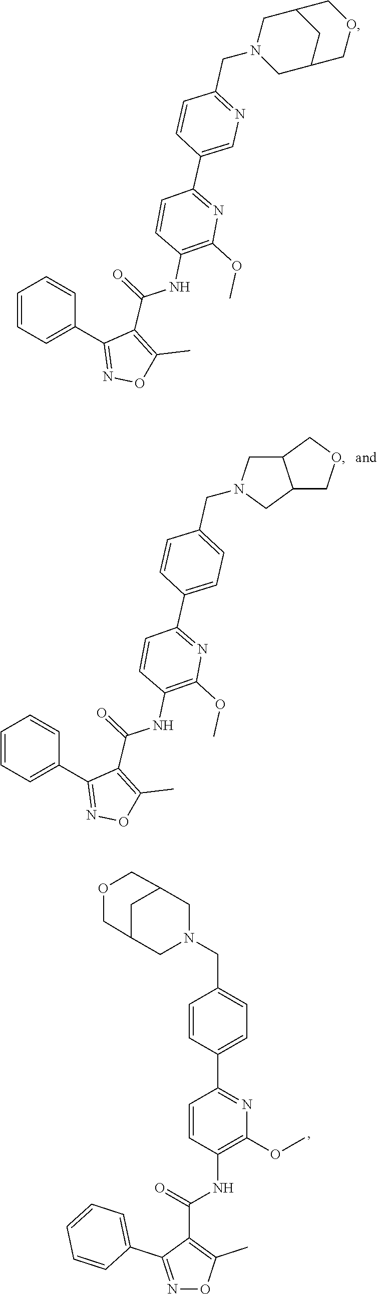 [SMILES: COc1nc(-c2ccc(CN3CC4COCC(C4)C3)nc2)ccc1NC(=O)c1c(-c2ccccc2)noc1C ; COc1nc(-c2ccc(CN3CC4COCC4C3)cc2)ccc1NC(=O)c1c(-c2ccccc2)noc1C ; "and" ; COc1nc(-c2ccc(CN3CC4COCC(C4)C3)cc2)ccc1NC(=O)c1c(-c2ccccc2)noc1C]

or a pharmaceutically acceptable salt thereof.

In another embodiment there is provided a compound selected from any one of the Examples herein, or a pharmaceutically acceptable salt thereof.

In another embodiment there is provided a compound selected from:

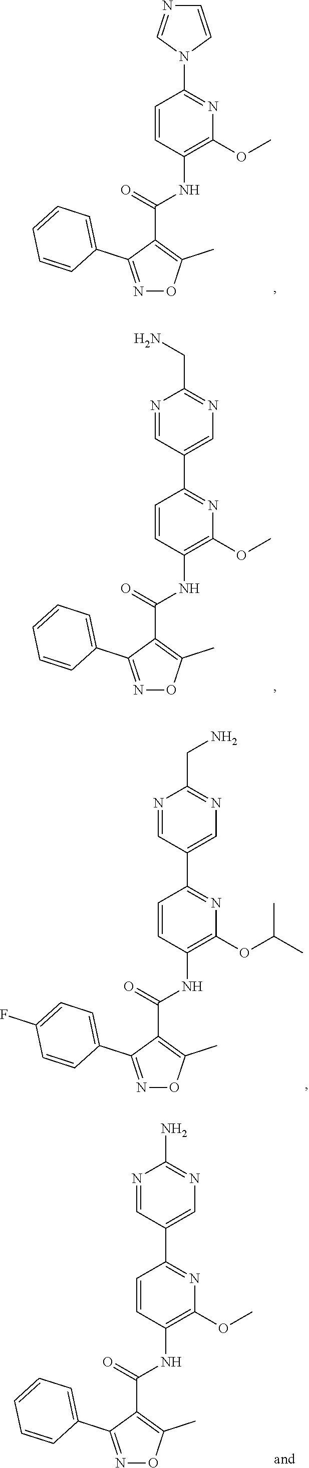

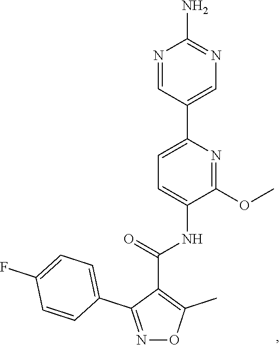 [SMILES: COc1nc(-c2cnc(N)nc2)ccc1NC(=O)c1c(-c2ccc(F)cc2)noc1C]

or a pharmaceutically acceptable salt thereof.

Particular compounds of the invention are those that have an affinity (Ki) for α5-GABA_{A}Rs of less than 30 nM (e.g. 10 nM, or less) when measured in the in vitro radioligand binding assay described herein. Preferred compounds of the invention have binding affinities and/or efficacy that are selective for α5-GABA_{A}Rs over GABA_{A} receptors containing α1, α2 or α3 subunits.

### Pharmaceutical Compositions

In accordance with another aspect, the present invention provides a pharmaceutical composition comprising a compound of the invention, or a pharmaceutically acceptable salt thereof, and a pharmaceutically acceptable excipient.

Conventional procedures for the selection and preparation of suitable pharmaceutical compositions are described in, for example, “Pharmaceuticals—The Science of Dosage Form Designs”, M. E. Aulton, Churchill Livingstone, 1988.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intraperitoneal dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of a condition is an amount sufficient to symptomatically relieve in a warm-blooded animal, particularly a human the symptoms of the condition or to slow the progression of the condition.

The amount of active ingredient that is combined with one or more excipients to produce a single dosage form will necessarily vary depending upon the host treated and the particular route of administration. For example, a formulation intended for oral administration to humans will generally contain, for example, from 0.1 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the invention will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, a daily dose selected from 0.1 mg/kg to 100 mg/kg, 1 mg/kg to 75 mg/kg, 1 mg/kg to 50 mg/kg, 1 mg/kg to 20 mg/kg or 5 mg/kg to 10 mg/kg body weight is received, given if required in divided doses. In general, lower doses will be administered when a parenteral route is employed. Thus, for example, for intravenous, subcutaneous, intramuscular or intraperitoneal administration, a dose in the range, for example, 0.1 mg/kg to 30 mg/kg body weight may be suitable. Similarly, for administration by inhalation, a dose in the range, for example, 0.05 mg/kg to 25 mg/kg body weight may be suitable. When administered orally a total daily dose of a compound of the invention may be, for example, selected from: 1 mg to 1000 mg, 5 mg to 1000 mg, 10 mg to 750 mg or 25 mg to 500 mg. Typically, unit dosage forms will contain about 0.5 mg to 0.5 g of a compound of the invention. In a particular embodiment the compound of the invention is administered parenterally, for example by intravenous administration. In another particular embodiment the compound of the invention is administered orally.

### Therapeutic Uses and Applications

In accordance with another aspect, the present invention provides a compound of the invention, or a pharmaceutically acceptable salt thereof, for use as a medicament.

A further aspect of the invention provides a compound of the invention, or a pharmaceutically acceptable salt thereof, for use in the prevention or treatment of a disease or medical disorder mediated by α5-GABA_{A}Rs.

Also provided is a method of preventing or treating a disease or medical disorder mediated by α5-GABA_{A}Rs in a subject, the method comprising administering to the subject an effective amount of a compound of the invention, or a pharmaceutically acceptable salt thereof.

Also provided is the use of a compound of the invention, or a pharmaceutically acceptable salt thereof, for the manufacture of a medicament for the prevention or treatment of a disease or medical disorder mediated by α5-GABA_{A}Rs.

In the following sections of the application reference is made to a compound of the invention, or a pharmaceutically acceptable salt thereof for use in the prevention or treatment of certain diseases or medical disorders. It is to be understood that any reference herein to a compound for a particular use is also intended to be a reference to (i) the use of the compound of the invention, or pharmaceutically acceptable salt thereof, in the manufacture of a medicament for the prevention or treatment of that disease or disorder; and (ii) a method for the prevention or treatment of the disease or disorder in a subject, the method comprising administering to the subject a therapeutically effective amount of the compound of the invention, or pharmaceutically acceptable salt thereof.

In certain embodiments there is provided a compound of the invention, or a pharmaceutically acceptable salt thereof, for use in the prevention or treatment of cognitive dysfunction associated with a disease or medical disorder mediated by α5-GABA_{A}Rs.

In certain embodiments there is provided a compound of the invention, or a pharmaceutically acceptable salt thereof, for use in the prevention or treatment of a neurological or neuropsychiatric disorder mediated by α5-GABA_{A}Rs. In certain embodiments there is provided a compound of the invention, or a pharmaceutically acceptable salt thereof, for use in the prevention or treatment of cognitive dysfunction associated with a neurological or neuropsychiatric disorder mediated by 5-GABA_{A}Rs. It may be that the neurological disorder is a neurodevelopment disorder (e.g. attention deficit disorder (ADHD), Down Syndrome, learning disabilities, cerebral palsy, autism or a speech disorder). It may be that the neurological condition is a neurodegenerative disorder (e.g. Alzheimer's disease, dementia, Parkinson's disease, Huntington's disease, amyotrophic lateral sclerosis (ALS) or Creutzfeldt-Jakob disease (CJD)). In a particular embodiment the neurological disorder is Huntington's disease).

It may be that the disease or medical disorder mediated by α5-GABA_{A}Rs is selected from: Alzheimer's disease, Parkinson's disease, Huntington's disease, cognitive dysfunction (e.g. cognitive dysfunction associated with: chemotherapy, an anaesthetic, a bacterial infection or a viral infection (e.g. HIV), memory deficit, age-related cognitive impairment (i.e., mild cognitive impairment, MCI), a bipolar disorder, autism, Down syndrome, neurofibromatosis type I, a sleep disorder, a disorder of circadian rhythms, amyotrophic lateral sclerosis (ALS), a psychotic disorder (e.g. schizophrenia, schizoaffective disorder, schizophreniform disorder, substance-induced psychotic disorder or paraphrenia), psychosis, post-traumatic stress disorder, an anxiety disorder, a generalized anxiety disorder, a panic disorder, a delusional disorder, an obsessive/compulsive disorder, an acute stress disorder, drug addiction, alcohol disorders (e.g. alcohol addiction), drug withdrawal symptoms, a movement disorder, restless leg syndrome, a cognition deficiency disorder, multi-infarct dementia, vascular dementia, a mood disorder, depression, a neuropsychiatric condition, attention-deficit/hyperactivity disorder, neuropathic pain, chronic neuroinflammation cognitive dysfunction associated with stroke, cognitive dysfunction associated with brain injury or trauma, cognitive dysfunction associated with a brain tumour and an attentional disorder.

In certain embodiments there is provided a compound of the invention, or a pharmaceutically acceptable salt thereof, for use in the prevention or treatment of Alzheimer's disease, Parkinson's disease, Huntington's disease, cognitive dysfunction (e.g. cognitive dysfunction associated with: chemotherapy, an anaesthetic or, a bacterial infection or a viral infection (e.g. HIV), memory deficit, age-related cognitive impairment (e.g. mild cognitive impairment, MCI), a bipolar disorder, autism, Down syndrome, neurofibromatosis type I, a sleep disorder, a disorder of circadian rhythms, amyotrophic lateral sclerosis (ALS), a psychotic disorder (e.g. schizophrenia, schizoaffective disorder, schizophreniform disorder, substance-induced psychotic disorder or paraphrenia), psychosis, post-traumatic stress disorder, an anxiety disorder, a generalized anxiety disorder, a panic disorder, a delusional disorder, an obsessive/compulsive disorder, an acute stress disorder, drug addiction, alcohol disorders (e.g. alcohol addiction), drug withdrawal symptoms, a movement disorder, restless leg syndrome, a cognition deficiency disorder, multi-infarct dementia, vascular dementia, a mood disorder, depression, a neuropsychiatric condition, attention-deficit/hyperactivity disorder, neuropathic pain, chronic neuroinflammation cognitive dysfunction associated with stroke, cognitive dysfunction associated with brain injury or trauma, cognitive dysfunction associated with a brain tumour and an attentional disorder.

In certain embodiments there is provided a compound of the invention, or a pharmaceutically acceptable salt thereof, for use in the prevention or treatment of cognitive dysfunction associated with Alzheimer's disease.

In certain embodiments there is provided a compound of the invention, or a pharmaceutically acceptable salt thereof, for use in the prevention or treatment of cognitive dysfunction associated with Huntington's disease.

Chromosome 15q11.2-13.1 duplication syndrome (Dup15q syndrome) is a rare disease that arises from duplications of the portion of 15q11.2-13.1 chromosome. Dup15q syndrome is characterized by hypotonia and gross and fine motor delays, variable intellectual disability, autism spectrum disorder, and epilepsy including infantile spasms. Excessive α5-GABA_{A} receptor function has been identified in Dup15q syndrome (Frohlich et al. Mechanisms underlying the EEG biomarker in Dup15q syndrome. Molecular Autism 10, 29 (2019)). A phase II clinical trial using basmisanil (a α5-GABA_{A}R NAM) to treat children with Dup15q syndrome is also planned (NCT05307679).

In certain embodiments there is provided a compound of the invention, or a pharmaceutically acceptable salt thereof, for use in the treatment of Dup15q syndrome.

It may be that the compound of the invention is for use in the treatment of a child with DUP15 g syndrome, for example a child aged between 2 and 11 years. It may be that the compound of the invention reduces or eliminates one or more neurodevelopmental features associated with Dup15q syndrome, for example, hypotonia, motor delays, or intellectual development (e.g. speech or language delays, impaired cognition, or problems with social interactions).

In certain embodiments there is provided a compound of the invention, or a pharmaceutically acceptable salt thereof, for use in the prevention or treatment of chemotherapy induced cognitive dysfunction in a subject in need thereof. Examples of chemotherapy that can induce cognitive dysfunction include amphetamines, dopamine agonists, ketamine, corticosteroids and anticonvulsants.

In certain embodiments the compounds of the invention are for use in the treatment of depression, for example the treatment of treatment-resistant depression.

Neuroinflammation can induce psychiatric and neurological symptoms, for example cognitive dysfunction, and treatment using a α5-GABA_{A}R NAM may be beneficial (Jacob, 2019 Frontiers in Molecular Neuroscience, 12 179). Accordingly, in certain embodiments there is provided a compound of the invention for use in the prevention or treatment of psychiatric and/or neurological symptoms associated with neuroinflammation. In certain embodiments a compound of the invention is for use in the treatment or prevention of cognitive dysfunction associated with neuroinflammation. It may be that the neuroinflammation is chronic neuroinflammation. It may be that the neuroinflammation is caused by or associated with, for example, stroke, a bacterial infection, a viral infection, a traumatic brain injury, or an autoimmune disease (e.g. systemic lupus erythematosus).

It is well known that bacterial or viral infections can result in both acute and chronic cognitive dysfunction in subjects that are, or have been infected with a bacteria or virus. In particular, viral infections can result in so called “post-viral syndrome” in which a subject that has been infected with a virus experiences chronic symptoms following the viral infection. Such symptoms include chronic fatigue, sore joints, cognitive impairment, or flu-like symptoms. There is increasing evidence that certain subjects infected with the SARS-CoV-2 virus experience chronic symptoms, including cognitive dysfunction in so called “long-COVID” or “post-COVID syndrome” (Raveendran, et al., 2021 Diabetes & metabolic syndrome, 15 (3), 869-875. A compound of the invention may therefore be useful in the treatment or prevention of cognitive dysfunction caused by or associated with a viral or bacterial infection.

In certain embodiments there is provided a compound of the invention for use in the treatment or prevention of psychiatric and/or neurological symptoms (particularly cognitive dysfunction) caused by or associated with a viral or bacterial infection.

In some embodiments the psychiatric and/or neurological symptoms (e.g. cognitive dysfunction) is caused by or associated with a bacterial infection (for example a bacteria selected from Chlamydia pneumoniae, Helicobacter pylori, Borrelia spp. (e.g. B. burgdorferi, B. mayonii, B. afzelii or B. garinii), Treponema pallidum, Streptococcus pneumoniae, Neisseria meningitidis, Haemophilus influenzae, Listeria monocytogenes, Brucella spp., Mycobacterium tuberculosis, Salmonella spp., and Rickettsia spp.).

In some embodiments the psychiatric and/or neurological symptoms (e.g. cognitive dysfunction) is caused by or associated with a viral infection. In some embodiments the psychiatric and/or neurological symptoms (e.g. cognitive dysfunction) is caused by or associated with infection with a virus selected from families Coronaviridae (e.g. Alphacoronavirus, Betacoronavirus, Gammacoronavirus and Deltacoronavirus), Picornaviridae (e.g. Enteroviruses, such as rhinoviruses, Human rhinoviruses (HRVs)), Flaviviridae (e.g. Zika virus (ZIKV), dengue (e.g. DENV 1-4), West Nile virus (WNV), yellow fever virus (YFV, e.g. yellow fever 17D virus), Japanese encephalitis virus (JEV), Hepatitis C virus (HCV), Filoviridae (e.g. Ebolavirus)), Togaviridae (e.g. Alphaviruses such as Chikungunya virus (CHIKV), Sindbis virus and Ross River virus), Herpesvirus (e.g. γ-herpesvirus, Human herpesvirus 8, herpesvirus 1, herpesvirus 2, varicella zoster virus, cytomegalovirus (CMV) and Epstein-Barr virus (EBV)), Adenoviridae (e.g. Human adenoviruses (HAdVs)) a Lentivirus (e.g. HIV), and an influenza virus.

In certain embodiments the psychiatric and/or neurological symptoms (e.g. cognitive dysfunction) is caused by or associated with a virus selected from cytomegalovirus (CMV); herpes simplex virus-1 (HSV-1), HIV, hepatitis, varicella zoster virus, Zika virus, Epstein-Barr virus or a corona virus (e.g. SARS-CoV-2), or a variant thereof.

In certain embodiments the psychiatric and/or neurological symptoms (e.g. cognitive dysfunction) is caused by or associated with a coronavirus (e.g. severe acute respiratory syndrome coronavirus (SARS-CoV), severe acute respiratory syndrome coronavirus-2 (SARS-CoV-2), or Middle East respiratory syndrome coronavirus (MERS-CoV)), or a variant thereof.

In certain embodiments the psychiatric and/or neurological symptoms (e.g. cognitive dysfunction) is caused by or associated with a coronavirus that causes severe acute respiratory syndrome (SARS), such as a SARS virus or MERS virus, e.g. SARS-CoV, SARS-CoV-2, or MERS-CoV. Preferably the viral infection is caused by or associated with SARS-CoV-2, or a variant thereof.

In certain embodiments a compound of the invention is for use in the treatment or prevention of cognitive dysfunction caused by or associated with a respiratory viral infection, for example severe acute respiratory syndrome (SARS). In a particular embodiment a compound of the invention is for use in the treatment or prevention of cognitive dysfunction caused by or associated with COVID-19.

In certain embodiments the compound of the invention is for use in the treatment or prevention of psychiatric and/or neurological symptoms (particularly cognitive dysfunction) in a subject that has a viral or bacterial infection. In this embodiment the psychiatric and/or neurological symptoms are caused by or associated with the bacterial or viral infection. In this embodiment it may be that the subject has an acute viral or bacterial infection. The infection may be a symptomatic bacterial or viral infection. Alternatively, the infection may be an asymptomatic bacterial or viral infection. In this embodiment it may be that the subject has a viral infection. In this embodiment it may be that the subject has a bacterial infection.

In certain embodiments the compound of the invention is for use in the prevention or treatment of psychiatric and/or neurological symptoms (particularly cognitive dysfunction) in a subject with post-viral infection syndrome or post-bacterial infection syndrome. In this embodiment it may be that the subject has a post-viral infection syndrome. In this embodiment it may be that the subject has a post-bacterial infection syndrome. In this embodiment the compound is administered to a subject after the initial acute bacterial or viral infection, for example where the subject has recovered from the initial acute symptoms of the viral or bacterial infection, and/or where the subject is substantially free from the infectious bacteria or virus (e.g. where the subject has a substantially no or very low viral/bacterial load following the initial infection). The bacterial/viral load in a subject can be determined using well-known methods, for example bacterial culture methods and/or suitable diagnostic tests such as PCR-based methods.

The initial (acute) infection may, for example, be with any of the bacteria or viruses described herein. The initial acute bacterial or viral infection may be a symptomatic bacterial or viral infection. Alternatively, the initial infection may be an asymptomatic bacterial or viral infection.

In a particular embodiment the compound of the invention is for use in the treatment of cognitive dysfunction in a subject, wherein the subject has developed the cognitive dysfunction following a SARS-CoV-2 infection. Thus it maybe that the compound of the invention is for use in the treatment or prevention of cognitive dysfunction caused by or associated with COVID-19. For example, it may be that the compound of the invention is for use in the treatment of a subject that has developed cognitive dysfunction following a COVID-19 infection.

### Post-Operative Cognitive Dysfunction (POCD)

Post-operative cognitive dysfunction refers to cognitive impairment after anaesthesia and surgery. POCD is a broadly recognised clinical phenomenon, encompassing acute or persistent deficits in attention, concentration, learning and memory following surgery that are not attributable to an overt complication or insult arising from the procedure.

POCD is a transient disturbance that generally resolves within 3 months in young patients. However, POCD in elderly patients occurs far more frequently, with 41% of patient's over 60 years of age showing symptoms 7-days after surgery. POCD in elderly patents also can be long-lasting with >10% of patients showing symptoms 3-months post-surgery. POCD is linked with an increased risk of death within both 3 months and 1 year post-surgery. POCD may also be a risk factor for the development or acceleration of dementia (Monk et al. Anesthesiology 2008, 108 (1), 18-30; Moller et al. The Lancet 1998, 351 (9106), 857-861; and Needham et al., British Journal of Anaesthesia, 2017 Volume 119, i115-i125). Cardiopulmonary bypass (CPB) surgery has the strongest link to POCD, not least because it is one of the most common medical conditions and procedures globally. CPB surgeries are performed approximately 200,000 times each year in the US alone, with an average incidence rate of 62 per 100,000 inhabitants in western European countries (Melly et al. J. Thorac. Dis. 2018, 10 (3), 1960-1967).

General anaesthetics have been implicated as a cause of POCD by the observation that the duration of anaesthesia is positively correlated with the incidence of postoperative cognitive deficits in patients (Moller 1998 supra). A single exposure to an anaesthetic can cause retrograde and anterograde memory deficits that persist for days to weeks in rodent models (Crosby et al., Anesth. Analg., 2005, 101 (5), 1389-92; and Culley et al. Anesth. Analg., 2003, 96 (4), 1004-9).

Many general anaesthetics act as positive allosteric modulators of inhibitory γ-aminobutyric acid type A receptors (GABA_{A}Rs) (Antkowiaket al., Curr. Opin. Anaesthesiol. 2016, 29 (4), 447-453). During anaesthesia, increased activity of GABA_{A}Rs contributes to the desired and profound neurodepressive properties of anaesthetic drugs. Following cessation of administration of the anaesthetic, positive allosteric modulation of GABA_{A}R function is rapidly reversed and it has therefore been assumed that receptor activity returns to baseline and GABA_{A}Rs do not contribute to undesirable prolonged cognitive dysfunction after anaesthesia (Belelli et al., Br. J. Pharmacol. 1996, 118 (3), 563-76). However, this hypothesis has been challenged recently by observations that α5-GABA_{A}R null mice showed no symptoms of cognitive deficit in a novel-object recognition experiment following exposure to isoflurane. In contrast, wild-type mice exhibited cognitive decline, implying a role for α5-GABA_{A}Rs in cognition deficits caused by anaesthetics (Zurek et al. Anesth. Analg. 2012, 114 (4), 845-855). Changes in long-term potentiation of rat amygdala brain slices were also observed following repeat, but not single, doses of isoflurane (Long et al., Neural Plast. 2016, 8524560-8524560).

A single treatment of mice with the injectable anaesthetic etomidate increased a tonic inhibitory current generated by α5-GABA_{A}Rs and cell-surface expression of α5-GABA_{A}Rs for at least 1 week (Zurek 2012, supra). The sustained increase in α5-GABA_{A}R activity impaired memory performance and synaptic plasticity in the hippocampus. Similarly, the inhaled anaesthetic isoflurane triggered a persistent increase in tonic current and cell-surface expression of α5-GABA_{A}Rs. Thus, α5-GABA_{A}R function does not return to baseline after the anaesthetic is eliminated, suggesting a mechanism to account for persistent memory deficits after general anaesthesia. The study also showed that memory performance in mice following a single etomidate treatment on the novel object recognition task was impaired up to 72 hours. However, despite the still elevated tonic current, at 1 week memory performance recovered. The sustained increase in tonic current may trigger compensatory changes that contribute to the recovery of memory performance, given that homeostatic plasticity has been widely demonstrated in the hippocampus. Inhibition of α5-GABA_{A}Rs using the α5-GABA_{A}R NAM L-655,708 completely reversed the memory deficits induced by etomidate. A further study suggested that α5-GABA_{A}Rs on non-pyramidal cells could be most important for the effects on LTP seen with etomidate, although other anaesthetics were not evaluated (Rodgers et al., The Journal of Neuroscience: the official journal of the Society for Neuroscience 2015, 35 (26), 9707-9716).

In aged rats anaesthetised with isoflurane, L-655,708 was able to prevent, but not reverse, cognitive deficits in a Morris-Water Maze experiment. This was in contrast to young rats, where the compound was able to both prevent and reverse the cognitive effects (Zhao et al., Neural Regen. Res. 2019, 14 (6), 1029-1036).

Additionally, benzodiazepine use is linked to an increased risk of development of POCD, with studies suggesting 2.5 mg of midazolam more than doubles the risk of developing POCD. As benzodiazepines are non-specific positive allosteric modulators of GABA_{A}Rs, this suggests over stimulation of these receptors could cause or exacerbate POCD.

POCD may also be associated with inflammation resulting from surgical procedures. A comprehensive review of the mechanism underpinning the inflammatory hypothesis for POCD has recently been published by Safavynia and Goldstein (Frontiers in psychiatry 2019, 9, 752-752). It is thought that peripheral surgical trauma causes CNS inflammation via disruption of the blood-brain barrier (BBB), which then causes a functional disruption in neural activity, leading to POCD. Further, TNFα can depress inhibitory neurotransmission via downregulation of GABA receptors, disrupting the delicate balance of excitatory and inhibitory neurotransmission and ultimately favouring glutamate toxicity (Pribiag et al., J. Neurosci. 2013, 33 (40), 15879-93).

Acute inflammation reduced long-term potentiation, a synaptic correlate of memory, in hippocampal slices from wild-type mice, and this reduction was reversed by inhibition of α5-GABA_{A}R function (Wang et al., Cell reports 2012, 2 (3), 488-496). A tonic inhibitory current generated by α5-GABA_{A}Rs in hippocampal neurons was increased by the key pro-inflammatory cytokine interleukin-1β through a p38 mitogen-activated protein kinase signalling pathway. Interleukin-13 also increased the surface expression of α5-GABA_{A} receptors in the hippocampus (Wang et al supra).

The currents evoked by GABA in cultured hippocampal and cortical neurons by etomidate or isoflurane were increased by pre-treatment with the inflammatory cytokine interleukin-1β. In vivo, the immobilizing properties of etomidate, but not isoflurane, were also increased by lipopolysaccharide, suggested that inflammation increases sensitivity to some anaesthetics (Avramescu et al., Anesthesiology 2016, 124 (2), 417-27).

LPS treatment significantly increased the duration of pentobarbital-induced loss of righting reflex (LORR) in mice treated with benzodiazepine site modulators (diazepam and brotizolam) and a GABA_{A}R agonist (muscimol) compared to that of mice treated with vehicle. These effects were blocked by bicuculline, a GABA_{A}R antagonist (Kitamura et al., European Journal of Pharmacology 2019, 842, 231-238). Inflammation-induced impairment using LPS of contextual fear memory was absent in Gabra5^{-/-} mice and was prevented by inhibition of α5-GABA_{A}Rs with L-655,708 in wild-type mice (Wang et al., Cell reports 2012, 2 (3), 488-496). Increased extracellular levels of GABA and of membrane expression of the alpha 5 subunit of GABA_{A} receptors has also been observed in rat models of neuroinflammation (Agusti et al., CNS Neurosci. Ther. 2017, 23 (5), 386-394).

According to a further aspect there is provided a compound of the invention, or a pharmaceutically acceptable salt thereof, for use in the treatment or prevention of POCD in a subject.

In certain embodiments the subject has or will undergo a major surgical procedure, for example a surgical procedure with a duration of at least one hour. Examples of surgical procedures include cardiac; vascular; ear, nose, and throat; plastic; gynaecological; orthopaedic; urological; or ophthalmological surgery. In certain embodiments the subject has or will undergo cardiac surgery, for example coronary bypass surgery. In certain embodiments the subject has or will undergo orthopaedic surgery, for example hip or knee replacement surgery.

In certain embodiments the subject is anesthetised using a general anaesthetic, for example an anaesthetic selected from: an intravenous anaesthetic (e.g. etomidate, propofol, fospropofol, a barbiturate (e.g. amobarbital, methohexital, thiamyal or thiopental), a benzodiazepine (e.g. diazepam, lorazepam or midazolam), a triazolobenzodiazepine (e.g. alprazolam), a thienotriazolodiazepine (e.g. brotizolam), dexmedetomidine, ketamine, and an opioid (e.g. fentanyl, alfentanil, remifentanil or sufentanil); and an inhaled anaesthetic (e.g. nitrous oxide, xenon, cyclopropane, or a volatile anaesthetic (e.g. a halogenated anaesthetic (e.g. halothane, enflurane, isoflurane, sevoflurane or desflurane)).

In certain embodiments the POCD is anaesthetic-induced POCD. For example POCD induced by or associated with a general anaesthetic (e.g. one or more of the general anaesthetics described above). In certain embodiments the POCD is inflammation-induced POCD, for example inflammation induced by or associated with major surgical procedures (for example one or more of the surgical procedures described above).

A compound of the invention, or a pharmaceutically acceptable salt thereof may be administered to the subject before, after or during the surgical procedure. In certain embodiments a compound of the invention, or a pharmaceutically acceptable salt thereof may be administered to the subject before surgery. In certain embodiments a compound of the invention, or a pharmaceutically acceptable salt thereof may be administered to the subject during surgery. In certain embodiments a compound of the invention, or a pharmaceutically acceptable salt thereof may be administered to the subject after the surgical procedure.

In certain embodiments the subject is at least 60 years old. In certain embodiments the subject is less than 60 years old, for example from 18 to 59.

The magnitude of cognitive dysfunction associated with surgery, and the effects of a compound of the invention in the treatment of POCD may be assessed using a suitable clinical scoring system. For example, the subject may be assessed and scored prior to and after surgery. For example, the subject may be scored 1 day, 2 days, 3 days, 4 days, 5 days, 6 days, or 7 days before surgery. The patient may be scored 1 day, 2 days, 3 days, 4 days, 5 days, 6 days, or 7 days, 1 month, 2 months, 3 months, 6 months or 12 months after the surgical procedure. The severity of the POCD in a subject may be determined by comparing the clinical scores before and after the surgical procedure. Similarly, the effect of a compound of the invention on the treatment or prevention of POCD may be determined by comparing the clinical scores before and after treatment with the compound of the invention. Suitable scoring systems for assessing cognitive function are well known and include, for example the CogState Brief Battery (CBB) (Maruff et al., 2013, BMC psychology vol. 1,1 30). Immediate memory, visuospatial, and delayed memory domains may be assessed with the Repeatable Battery for the Assessment of Neuropsychological Status (RBANS) (Karantzoulis et al., 2013, Arch. Clin. Neuropsychol.; 28 (8): 837-44). Verbal fluency and executive function may be assessed using the Controlled Oral Word Association Test (COWAT) (Malek-Ahmadi et al., 2011, Dement. Geriatr. Cogn. Disord.; 32 (4): 235-40). Visuospatial and executive function may be assessed using the Trail Making Tests A and B (TMT) (Terada et al., 2013, Psychiatry Res.; 213 (3): 249-55).

### Selectivity

Non-selective inhibition of GABA_{A}Rs can result in undesirable side-effects, for example convulsant and/or anxiogenic effects. Selective α5-GABA_{A}R inhibition is expected to provide beneficial therapeutic effects, for example improved cognition, whilst avoiding or minimising the risk of undesirable side effects associated with non-selective GABA_{A}R inhibition (e.g. convulsant and/or anxiogenic effects).

Accordingly, preferred compounds of the invention, have selective affinity for, and/or attenuate the function of, α5 subunit containing GABA_{A}Rs over GABA_{A}Rs that do not contain an α5 subunit. For example, a compound of the invention has a selective affinity for, and/or selectively inhibits the function of, α5-GABA_{A}Rs over GABA_{A}Rs that contain α1, α2, α3, α4 or α6 subunits. In certain embodiments a compound of the invention has an affinity (Ki) for α5-GABA_{A}Rs that is at least 5-fold, at least 10-fold, at least 15-fold, at least 20-fold, at least 50-fold, at least 100-fold, at least 500-fold, or at least 1000-fold lower (e.g. about 20 to about 1000 fold lower) than the Ki for GABA_{A} receptors containing α1-, α2-, or α3-subunits when measured using the in vitro radioligand binding assay described herein.

In some embodiments compounds of the invention exhibit α5GABA_{A}R NAM activity when measured in the in vitro electrophysiological recording assay described herein. Preferred compounds of the invention selectively inhibit the function of α5-GABA_{A}Rs over GABA_{A}Rs containing α1, α2 or α3 subunits when measured in the in vitro electrophysiological recording assay described herein.

Preferred compounds of the invention have selective affinity for α5-GABA_{A}Rs and selectively inhibit the function of α5-GABA_{A}R over GABA_{A}Rs containing α1-, α2- or α3-subunits.

In certain embodiments such selective compounds may be used in the treatment or prevention of any of the diseases or medical conditions described herein.

### Combination Therapies

The compounds of the invention may be used alone to provide a therapeutic effect. The compounds of the invention may also be used in combination with one or more additional therapeutic agents.

In some embodiments the additional therapeutic agent is selected from one or more of:
an anti depressant, for example a tricyclic antidepressant (e.g. desipramine, imipramine, amitriptyline or nortriptyline);
a serotonin reuptake inhibitor, for example sertraline, demethylsertraline or fluoxetine;
a typical or atypical antipsychotic;
an inhibitor of acetylcholinesterase (e.g. galantamine or donepezil);
an anti-inflammatory drug;
a GABA_{A}R modulator other than a compound of the invention, for example a GABA_{A}R modulator acting via alternative binding sites on the GABA_{A}R receptor, or a GABA_{A}R modulator acting via alternative subunits on the GABA_{A}R;
an antiepileptic drug (e.g. acetazolamide, brivaracetam, cannabidiol, carbamazepine, clobazam, clonazepam, eslicarbazepine, ethosuximide, everolimus, gabapentin, lacosamide, lamotrigine, levetiracetam, oxcarbazepine, padsevonil perampanel, phenobarbital, phenytoin, piracetam, pregabalin, primidone, pufinamide, sodium valproate, stiripentol, tiagabine, topiramate, valproic acid, vigabatrinor or zonisamide); or
an agent that inhibits the rate of metabolism of a compound of the invention.

Such combination treatment may be achieved by way of the simultaneous, sequential or separate dosing of the individual components of the treatment. Such combination products employ the compounds of this invention within a therapeutically effective dosage range described hereinbefore and the other pharmaceutically-active agent within its approved dosage range.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

In some embodiments in which a combination treatment is used, the amount of the compound of the invention and the amount of the other pharmaceutically active agent(s) are, when combined, therapeutically effective to treat a targeted disorder in the patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound of the invention and an approved or otherwise published dosage range(s) of the other pharmaceutically active compound(s).

### Biological Assays

The biological effects of the compounds may be assessed using one of more of the assays described herein.

### Cell Lines

Mouse L(tk⁻) cells stably expressing human α1β3γ2, α2β3γ2, α3β3γ2, α5β3γ2 GABA_{A}Rs generated by transfection of the individual subunits in the dexamethasone-inducible expression vector pMSGneo in mouse L(tk⁻) cells (Hadingham et al., 1993, Mol. Pharmacol. 43:970-975 and 1993, Mol. Pharmacol. 44:1211-1218) were used for binding and functional assays. For certain compounds, HEK293 cells stable expressing the human α1β3γ2L, α2β3γ2L, α3β3γ2L and α5β3γ2L GABA_{A} receptors were used to evaluate their functional effects in a SyncroPatch electrophysiology assay.

### Cell Culture

L(tk⁻) cells stably expressing human α1β3γ2, α2β3γ2, α3β3γ2, α5β3γ2 GABA_{A}Rs were maintained in DMEM F12 medium supplemented with 10% Foetal Bovine Serum, 1% Penicillin/Streptomycin and 1 mg/mL Geneticin G418 in an incubator at 37° C. with a humidified atmosphere with 5% CO₂.

For QPatch electrophysiology testing, 1 μM dexamethasone was added to the culture medium of L(tk⁻) cells 24 hours before the recordings were performed. Cells were typically used for electrophysiology experiments 72-96 hours after splitting.

### In Vitro Radioligand Binding Assay (α5-GABA_{A}R Ki)

The affinity (Ki) of compounds for the benzodiazepine site of human recombinant GABA_{A}Rs was measured by their ability to inhibit the binding of the selective benzodiazepine antagonist [³H] Ro15-1788 ([³H] flumazenil).

L(tk⁻) cells expressing human recombinant GABA_{A}R containing α1β3γ2, α2β3γ2, α3β3γ2, α5β3γ2 subunits were harvested and membranes were prepared for each receptor combination in phosphate buffer (K₂PO₄ 10 mM, pH 7.0) (Hadingham et al. (1992) Proc. Natl. Acad. Sci. USA; 89 (14): 6378-82). Protein concentration, receptor expression and the Kd of [³H] Ro15-1788 were determined before Ki values of compounds were evaluated. For Kd evaluation, saturation binding curves were obtained by incubating membrane with various concentrations of [³H] Ro15-1788 (82.5 Ci/mM), with nonspecific binding measured in the presence of 1 μM TP003.

[³H] Ro15-1788 ([³H] flumazenil) is tritiated on the N-methyl group as shown:

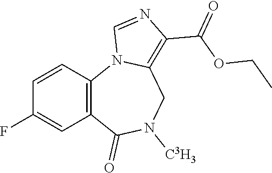

TP003 is a non-selective GABA_{A}R benzodiazepine site agonist of the formula:

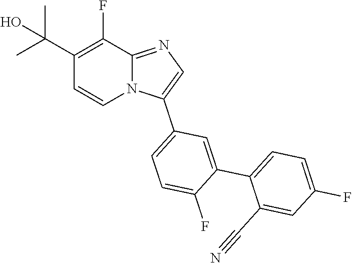 [SMILES: CC(C)(O)c1ccn2c(-c3ccc(F)c(-c4ccc(F)cc4C#N)c3)cnc2c1F]

For Ki evaluation, cell membranes were incubated with 4 nM [³H] Ro15-1788 along with a range of concentrations of test compound. Nonspecific binding was determined using 1 μM TP003. All incubations were performed for 1 hour at 4° C. in assay buffer. The total assay volume was 0.5 mL, containing 100 μg membrane protein/well α1β3γ2, α2β3γ2, α3β3γ2 and 50 μg membrane protein/well for α53γ2. Incubations were terminated by filtration and washing with ice cold Tris-HCl buffer (50 mM, pH=7.4) over Whatman GF/B filters and the radioactivity of the filters was measured using liquid scintillation counting.

The % inhibition of [³H] Ro15-1788 binding was plotted as a function of compound concentration and the IC₅₀ calculated. From the IC₅₀, the affinity (Ki) was calculated using the method of Cheng and Prusoff using the Kd values obtained for [³H] Ro15-1788.

The compounds of the present invention tested in the above described assay were found to have affinity for α5-GABA_{A}Rs. Preferred compounds have a Ki<30 nM and selectivity (20-1000-fold) for the α5-over the α1-, α2- and α3-GABA_{A}Rs.

### In Vitro Electrophysiological Recording Assay (α5-GABA_{A}R Relative Efficacy)

The efficacy of modulators was evaluated using either the automated patch-clamp platform QPatch16 (Sophion, Copenhagen, Denmark) or the SyncroPatch 384i (Nanion Technologies, Germany). Cells were harvested from flasks by enzymatic dissociation and re-suspended in serum-free medium.

For QPatch recordings, all experiments were carried out at room temperature (20-22° C.) using a standard whole cell procedure and physiological solutions. Gigaseals were formed upon execution of a combined suction/voltage protocol with subsequent increased suction leading to the whole-cell configuration. The currents recorded were acquired at 1 KHz and filtered at 0.3 KHz using a Bessel filter. Whole-cell currents were measured at a holding potential of −65 mV.

For the Syncropatch recordings, a stacked addition protocol was used, in which GABA was rapidly applied and then washed off from the cell. All experiments were carried out at room temperature (20-22° C.) using a standard whole cell procedure. The currents recorded were acquired at 2 kHz and filtered using a Bessel filter. Whole-cell currents were measured at a holding potential of −80 mV.

For QPatch recordings, the extracellular solution contained of (in mM): 145 NaCl, 4 KCl, 1 MgCl₂, 2CaCl₂, 10 HEPES, 10 D-glucose (pH 7.4), and the intracellular solution consisted of (in mM): 96 KCl, 28 CsCl, 25 KOH, 4.3 CaCl₂), 1.4 MgCl₂, 10 EGTA, HEPES, 3 MgATP (pH 7.2). The osmolarities of the extracellular and intracellular solutions were 305 and 295 mOsm respectively.

For Syncropatch recordings, the extracellular recording solution contained: 140 mM NaCl, 4 mM KCl, 2 mM CaCl₂), 1 mM MgCl₂, 10 mM HEPES, 5 mM glucose (pH 7.4 and osmolarity of c. 300-310 mOsm/L). The intracellular recording solution contained: 90 mM KCl, 50 mM KF, 1.5 mM MgCl₂, 11.1 mM EGTA and 10 mM HEPES (pH 7.2 and osmolarity of c. 300 mOsm/L). 2 mM of NaATP was added to the intracellular solution on the day of testing.

The effects of modulators were evaluated in the presence of a submaximal GABA concentration, giving typically 10-20% activation (GABA EC₁₀₋₂₀) of the response elicited by a saturating GABA concentration. For QPatch recordings, to check and ensure baseline current stability before compound addition, five consecutive applications of GABA EC₁₀₋₂₀ alone (each application with a 2 s duration, separated by minimum 1 min wash off periods) were performed before compound addition. The test compound was applied using the pipetting system of the QPatch system 1 min at least prior to co-application with GABA EC₁₀₋₂₀. For SyncroPatch recordings, GABA EC₂₀ alone was applied three times (1 s long applications, with wash steps in-between) before compound addition. This was followed by a 1-2 min pre-incubation with the test compound and then re-application of GABA EC₁₀₋₂₀ in the presence of the compound. Finally, following a further wash step, a saturating concentration of GABA was applied, allowing the accurate evaluation for each cell of the percentage baseline activation elicited by the submaximal GABA applied.

The compounds were first dissolved in DMSO as a 10 mM stock and then further diluted to the testing concentrations so that the final DMSO concentration in the extracellular recording solution was kept constant at 0.1% for the QPatch recordings, or 0.2% for the SyncroPatch recordings.

The % efficacy of modulators was determined from the GABA elicited currents recorded in the presence and absence of the test compound, using the formula:

[((compound peak current−leak)−(GABA peak current−leak))/(GABA peak current−leak)]*100,

where ‘leak’ is the leak baseline current at −65 mV, ‘compound peak current’ is the current elicited by co-application of compound and GABA, and ‘GABA peak current’ is the current elicited by GABA alone during the 5th GABA application. The results were presented as ‘relative efficacy’ for each compound generally at a concentration equal or higher than 100 times their determined Ki. The relative efficacy of a compound was calculated by normalising its efficacy to the efficacy of methyl-6,7-dimethoxy-4-ethyl-beta-carboline-3-carboxylate (DMCM) that was separately determined produced an attenuation of GABA-induced currents of −57±4%, n=5.

The compounds of the invention tested in the above described assays were found to possess α5-GABA_{A}R NAM activity and selectivity for the α5 subtype over the α1, α2, and α3.

### Electrophysiology In Hippocampal Slices-Long Term Potentiation Assay

Hippocampal long-term potentiation (LTP) is a form of synaptic plasticity associated with both learning and memory formation. The intravenous general anaesthetic etomidate, when applied acutely to an in vitro mouse hippocampal slice preparation, impairs LTP of CA1 pyramidal neurons. This effect of etomidate is reversed by α5-GABA_{A}R NAMs and does not occur in equivalent recordings made from α5^{-/-} mice, implicating a crucial role for α5-GABA_{A}Rs in this form of synaptic plasticity (Martin et al. 2009, Anesthesiology; 111:1025-1035; Zurek et al. 2014, J Clin Invest; 124 (12): 5437-5441). Here the efficacy of α5-GABA_{A}R NAMs was assessed by determining their ability to rescue LTP suppressed by the presence of etomidate.

Wild type male mice (C57BI6J) aged 2-5 months were decapitated following cervical dislocation, and their brains quickly removed and placed in ice-cold, oxygenated (95% O₂, 5% CO₂) artificial cerebrospinal fluid (aCSF) (composition in mM: 124 NaCl, 3 KCl, 1.75 MgCl₂, 1 CaCl₂), 1.25 NaH₂PO₄, 26 NaHCO₃, and 10 D-glucose) with the osmolarity of the aCSF adjusted to 300-310 mOsm. Brain slices (400 μm) containing sagittal sections of the hippocampus were prepared with a VT 1000E tissue slicer (Leica). Following a recovery period of 1 hr in the oxygenated aCSF, the slices were transferred to a submersion recording chamber where they were continually perfused with oxygenated aCSF containing 1 mM MgCl₂ and 2.5 mM CaCl₂).

For electrophysiological recordings a single slice was transferred to a submerged recording chamber (Scientific Systems Design, Mississauga, Ontario, Canada). The oxygenated aCSF solution, was maintained at 32° C. by a temperature controller (Digitimer Proportional Temperature Controller PTC03). The slice was oxygenated in the perfusion system with aCSF (flow rate ˜2 ml/min).

To monitor basal synaptic transmission a bipolar stimulating electrode, either hand-made from twisted Teflon-coated tungsten wire (Advent research materials, Ltd, Eynsham, Oxfordshire, UK), or a commercial electrode (World Precision Instruments, Florida, USA), was used to stimulate the Schaffer collateral-commissural pathway from area CA3 to the CA1 region of the hippocampus. The stimulus was delivered to the slice every 30 sec to record dynamic changes in the neurally-evoked field excitatory postsynaptic potential (fEPSP). The stimulus was delivered by a constant current isolated electronic stimulator (Digitimer Ltd, model DS2, Hertfordshire, UK). The stimulatory current was adjusted to produce a response with a fEPSP slope that was 40% of the maximum population spike-free response. The fEPSPs were recorded using an aCSF-filled glass borosilicate microelectrode (Kind precision glass, Inc., Claremont, USA), and placed in the apical dendritic layer of the CA1 pyramidal cells.

For control LTP experiments the fEPSP resulting from a stimulus delivered 1/30 sec was monitored for 15-20 min to ensure stability of the recording (fEPSP slope and amplitude) prior to inducing LTP. To subsequently induce control maximal LTP a theta-burst stimulation (TBS) protocol was delivered (4 pulses at 100 Hz, repeated 10 times, at an interval of 200 ms between the groups of 4 pulses; 4-TBS), before returning to a single stimulus delivered again at 1/30 sec.

The fEPSP measurements (1/30 sec) were monitored for an additional 60 min after delivery of the 4-TBS. Analysis of fEPSPs was performed using WinLTP software (Anderson, https://www.winltp.com/). Statistical analysis of LTP was performed using GraphPad Prism statistical software and comparisons of drug effects on the magnitude of LTP were measured by means of one-way ANOVA at 50-60 min after the 4-TBS and compared to the control baseline fEPSPs.

The effect on LTP by etomidate alone, or etomidate in the presence of an α5-GABA_{A}R NAM, was investigated by applying vehicle, etomidate, or etomidate+α5-GABA_{A}R NAM for at least 30 min before delivering the 4-TBS.

Etomidate was dissolved in H₂O as a 10 mM stock. Stock solutions of α5-GABA_{A}R NAMs (10 mM; in 100% DMSO) were diluted as required with a max final concentration of the vehicle 0.01%.

Certain compounds of the invention were tested in the above assay and were found to restore partially or fully the LTP suppression caused by etomidate.

### In Vivo Brain Receptor Occupancy Assay

The occupancy of rat brain benzodiazepine binding sites by compound was measured by its ability to inhibit the in vivo binding of [³H] L-655,708. This radioligand is selective for the benzodiazepine site of GABA_{A} receptors containing an α5 subunit (Quirk et al. 2001, Journal of Neurochemistry, 77 445-51) and has been used previously to measure in vivo α5-GABA_{A}R occupancy (Atack et al. 2005, Neuropharmacology, 49 220-229).

Male Sprague-Dawley rats (250-310 g, 4-6 per group) were dosed with either the vehicle (0.5% methylcellulose) or the test compound suspended in vehicle (1, 3 and 10 mg/kg) per os (5 ml/kg), 30 min to 4 hours before killing. To block all benzodiazepine binding sites and therefore define the level of non-specific binding of [³H] L-655,708, a separate group of animals received a dose of 10 mg/kg p.o. of TPA023 (5 ml/kg) (Atack et al. 2008 CNS Neuroscience Therapeutics 14 25-35) made up in 0.5% methylcellulose. Animals pre-treated with compounds received a tail-vein injection of [³H] L-655,708 (0.5 μL/g, 30 Ci/mM, 1:60 dilution in 0.9% NaCl solution) one minute prior to killing.

Animals were then killed by stunning and decapitation, the whole brain was rapidly removed, the cerebellum and brainstem were discarded, leaving the forebrain which was weighed and homogenised in 10 volumes of ice-cold homogenising buffer (10 mM potassium phosphate buffer containing 100 mM KCl, pH 7.4). Aliquots of homogenate (500 μL) were then added directly to scintillation vials (total radioactivity) or filtered and washed with 5 ml Tris-HCL buffer (50 mM, pH 7.4) over Whatman glass microfiber GF/B filters. Washed filters were then placed in scintillation vials (three filters per vial) and scintillation fluid was added to all vials, which were then counted on a Perkin-Elmer Tricarb 2900TR scintillation counter (membrane-bound radioactivity).

The percent occupancy was defined as the percentage by which specific binding in the vehicle treated group was inhibited by drug treatment. Therefore, the in vivo binding of the modulator in drug-treated animals was expressed as: % binding=[(cpm vehicle−cpm sample)/(cpm vehicle−cpm NSB)]*100, where ‘cpm vehicle’, ‘cpm sample’ and ‘cpm NSB’ are the average counts in vehicle, modulator and TPA023-treated animals, respectively.

Certain compounds of the invention were tested in the above described assay and showed in vivo brain 5-GABA_{A}R engagement when dosed orally.

### Other In Vivo Assays

The in vivo biological effects of the compounds may be assessed using other assays, some of which may be the T-maze spontaneous alternation task, a widely used behavioural test to assess the cognitive ability of rodents and performing in vivo EEG recordings that give a measure of engaged brain networks.

The spontaneous alternation task in the T-maze is a hippocampal dependent task that is sensitive to various pharmacological manipulations that affect memory processes and α5-GABA_{A}R NAMs may be tested in this assay for reversing induced cognitive deficits (Gerlai, 1998 Behav. Brain Res.; 95 (1): 91-101; Andriambeloson et al., 2014, Pharmacol. Res. Perspect; 2 (4): e00048).

Performing in vivo EEG recordings from rodents may be another suitable assay to assess the effects of α5-GABA_{A}R NAMs on EEG power spectra in conscious rodents reflecting in vivo changes in brain activity (Zanos et al., 2017, eNeuro; 4 (1): ENEURO.0285-16.2017)

### Synthesis

In the description of the synthetic methods described below and in the referenced synthetic methods that are used to prepare the staring materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl or trifluoroacetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃·OEt₂. A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, or sodium hydroxide, or ammonia. Alternatively, an arylmethyl group such as a benzyl group may be removed, for example, by hydrogenation over a catalyst such as palladium on carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

### General Synthetic Routes

Compounds of the invention may be prepared by a number of synthetic routes, including but not limited to the following.

Compounds of the formula (I) wherein Ring B is linked to the remainder of the compound if formula (I) by a ring nitrogen in Ring B, may be prepared by reacting a compound of the formula (A):

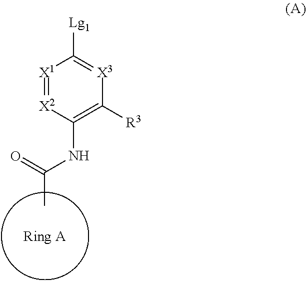

wherein Lg1 is a suitable leaving group; and
Ring A, R³ X¹, X² and X³ have any of the meanings defined herein, except that any functional group is protected if necessary, with a compound of the formula (B):

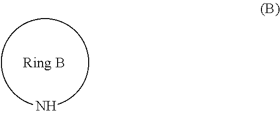

wherein Ring B has any of the meanings defined herein, except that any functional group is protected if necessary, provided Ring B has an —NH— group;
   and optionally thereafter carrying out one or more of the following procedures:
   converting a compound of formula (I) into another compound of formula (I); and/or
   removing any protecting groups; and/or
   forming a pharmaceutically acceptable salt.

Lg1 is a suitable leaving group, for example halo, particularly Br or I. Suitably the reaction is carried out in a suitable solvent (e.g. DMSO, DMF or NMP) in the presence of a suitable catalyst (e.g. copper(I) iodide and L-proline) and a suitable base (e.g. potassium carbonate or potassium phosphate tribasic).

Compounds of the formula (A) may be prepared using, for example the Reaction Scheme 1:

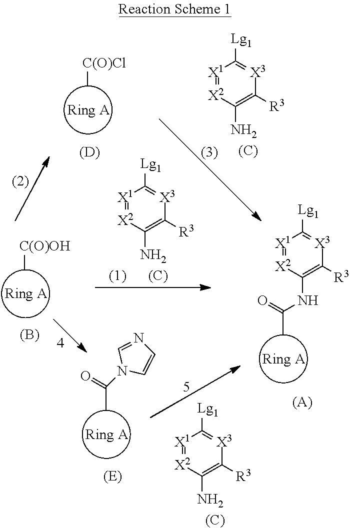

wherein Ring A, R³ X¹, X² and X³ have any of the meanings defined herein, except that any functional group is protected if necessary; and Lg1 is a suitable leaving group.

### Notes for Reaction Scheme 1

(1): The carboxylic acid (B) is coupled with the aniline (C). The reaction is suitably performed in the presence of a suitable activating agent (e.g. EDC, HATU, HBTU, PyBroP or T3P). The coupling reaction is suitably performed in the a suitable solvent (e.g. DCM, DMF, THF or EtOAc) and in the presence of a suitable base (e.g. an organic amine, for example triethylamine, N, N-diisopropylethylamine or DMAP).
(2): The carboxylic acid (B) may be converted to the corresponding acid chloride (D) using well known methods. For example by reacting (B) with a suitable reagent, for example thionyl chloride or oxalyl chloride in a suitable solvent (e.g. DCM or toluene). Optionally the reaction is carried out in the presence of a suitable catalyst (e.g. DMF).
(3): The acid chloride (D) is reacted with the aniline (C). The reaction is suitably performed in a suitable solvent (e.g. DCM or THF), in the presence of a suitable base (e.g. an organic amine (e.g. triethylamine or N,N-diisopropylethylamine) or a carbonate base (e.g. potassium carbonate).
(4): Carboxylic acid (B) is reacted with an suitably activating agent such as 1,1′-Carbonyldiimidazole (CDI) in a suitable solvent such as THF, 1,4-dioxane or DCM to give the CDI adduct (E).
(5) The adduct (E) is reacted aniline in the presence of a suitable base (e.g. lithium bis(trimethylsilyl) amide), in a suitable solvent (e.g. THF or dioxane) to give compound (A).

The carboxylic acids (B), anilines (C) and Ring B are commercially available, or may be prepared using well-known methods. For example, carboxylic acids (B) may be prepared using analogous methods to those described in the literature (for example WO2018/104419, WO 2012/062687, WO2010/127978 and Cheng H. M. et al. 2012 Journal of Medicinal Chemistry 55 2144-2153).

Compounds of the formula (I) wherein Ring B is linked to the remainder of the compound if formula (I) by a ring carbon atom in Ring B, may be prepared by coupling a compound of the formula (A1):

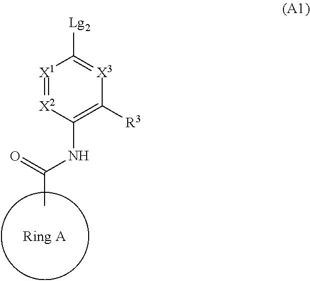

wherein Lg2 is halo or triflate; and
Ring A, R³ X¹, X² and X³ have any of the meanings defined herein, except that any functional group is protected if necessary, with a compound of the formula (F):

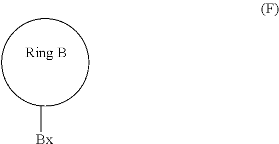

wherein Ring B has any of the meanings defined herein, except that any functional group is protected if necessary, and
   Bx is boronic acid or an ester thereof, or a trifluoroborate salt;
   and optionally thereafter carrying out one or more of the following procedures:
   converting a compound of formula (I) into another compound of formula (I); and/or
   removing any protecting groups; and/or
   forming a pharmaceutically acceptable salt.

The coupling reaction is suitably performed using the Suzuki coupling reaction. Suitably, the coupling reaction is carried out in the presence of a metal catalyst, for example a palladium catalyst, such as [1,1′-Bis(di-tert-butylphosphino) ferrocene] dichloropalladium (II) or [1,1′-Bis(diphenylphosphino) ferrocene] dichloropalladium (II)), and a suitable base (e.g. a carbonate (potassium or caesium carbonate), potassium phosphate tribasic, potassium acetate or an organic amine base (e.g. triethylamine). The coupling reaction is suitably performed in a suitable solvent (e.g. toluene, THF, dioxane, dimethoxyethane or water).

The group Bx may be boronic acid (—B(OH)₂). Alternatively, Bx may be a boronic acid ester, for example a catechol boronic ester, Pinacol boronic ester, an alkyldiol ester (e.g. 1,3-propanediol ester or neopentylglycol ester). In certain embodiments Bx is a trifluoroborate salt (e.g. a potassium trifluoroborate salt).

Compounds of the formula (F) are commercially available or can be prepared using well-known methods.

Compounds of the formula (I) wherein Ring B is linked to the remainder of the compound if formula (I) by a ring carbon atom in Ring B, may be prepared by coupling a compound of the formula (G):

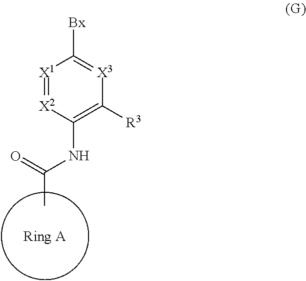

wherein Bx is boronic acid or an ester thereof, or a trifluoroborate salt; and
Ring A, R³ X¹, X² and X³ have any of the meanings defined herein, except that any functional group is protected if necessary, with a compound of the formula (H):

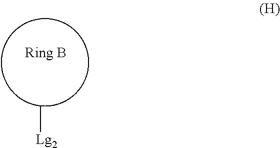

wherein Ring B has any of the meanings defined herein, except that any functional group is protected if necessary, and
   Lg2 is halo or triflate;
   and optionally thereafter carrying out one or more of the following procedures:
   converting a compound of formula (I) into another compound of formula (I); and/or
   removing any protecting groups; and/or
   forming a pharmaceutically acceptable salt.

The coupling reaction may be carried out using analogous methods to those described above for the coupling of (A) and (F).

Compounds of the formula (G) may be prepared by converting a compound of the formula (A) into its corresponding boronic acid or boronate ester using an appropriate boron reagent (e.g. 4,4,5,5-tetramethyl-2-(4,4,5,5-tetramethyl-1,3,2-dioxaborolan-2-yl)-1,3,2-dioxaborolane) in the presence of a suitable catalyst (e.g. [1,1′-Bis(diphenylphosphino) ferrocene] dichloropalladium (II)) and a suitable base (e.g. potassium acetate). The reaction is suitably performed in the presence of a suitable solvent (e.g. dioxane or DMSO). Compounds of the formula (H) are commercially available or can be prepared using well-known methods.

Compounds of the formula (I) wherein may also be prepared according to Reaction Scheme 2:

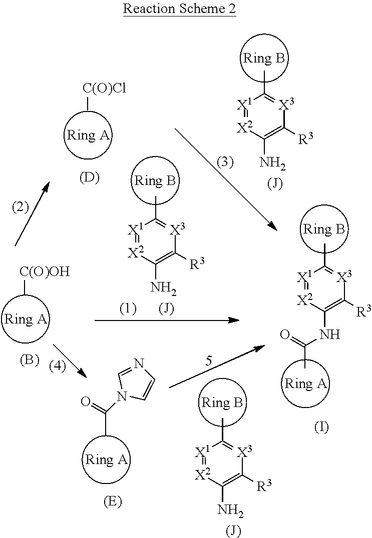

wherein Ring A, Ring B, R³ X¹, X² and X³ have any of the meanings defined herein, except that any functional group is protected if necessary;
   and optionally thereafter carrying out one or more of the following procedures:
   converting a compound of formula (I) into another compound of formula (I); and/or
   removing any protecting groups; and/or
   forming a pharmaceutically acceptable salt.

Reaction conditions for steps (1), (2), (3), (4) and (5) are analogous to those described for Reaction Scheme 1 above.

Compounds of the formula (J) are commercially available or may be prepared using well known methods, such as those described in the Examples herein.

### EXAMPLES

### Abbreviations

| | |
|---|---|
| B₂pin₂ | Bis(pinacolato)diboron |
| CV(s) | Column volume(s) |
| DCM | Dichloromethane |
| Dioxane | 1,4-Dioxane |
| DIPEA | N,N-Diisopropylethylamine |
| DMAP | 4-(Dimethylamino)pyridine |
| DMF | N,N-Dimethylformamide |
| DMSO | Dimethylsulfoxide |
| dppf | 1,1′-Bis(diphenylphosphino)ferrocene |
| EDC/EDC•HCl | N-(3-Dimethylaminopropyl)-N′-ethylcarbodiimide |
| | (hydrochloride) |
| Et₃N/TEA | Triethylamine |
| EtOAc | Ethyl acetate |
| EtOH | Ethanol |
| Et₂O | Diethyl ether |
| h/hr/hrs | Hour(s) |
| HATU | 1-[Bis(dimethylamino)methylene]-1H-1,2,3-triazolo[4,5- |
| | b]pyridinium 3-oxid hexafluorophosphate |
| HBTU | N,N,N′,N′-Tetramethyl-O-(1H-benzotriazol-1-yl)uronium |
| | hexafluorophosphate |
| HCl | Hydrochloric acid |
| Hunig's base | N,N-Diisopropylethylamine |
| K₂CO₃ | Potassium carbonate |
| KF | Potassium fluoride |
| KOAc | Potassium acetate |
| KOH | Potassium hydroxide |
| LCMS/LC-MS | Liquid chromatography-mass spectrometry |
| LiOH | Lithium hydroxide |
| MDAP | Mass directed autoprep system |
| MeCN | Acetonitrile |
| MeOH | Methanol |
| Min/mins | Minute(s) |
| Na₂CO₃ | Sodium carbonate |
| NaHCO₃ | Sodium bicarbonate |
| NaOH | Sodium hydroxide |
| NH₃ | Ammonia |
| NH₄Cl | Ammonium chloride |
| NMR | Nuclear magnetic resonance |
| Na₂SO₄ | Sodium sulfate |
| o/n | Overnight |
| Pd-118 | [1,1′-Bis(di-tert-butylphosphino)ferro- |
| | cene]dichloropalladium(II) |
| Pd(dppf)Cl₂ | [1,1′-Bis(diphenylphosphino)ferro- |
| | cene]dichloropalladium(II) |
| PE/petroleum | Petroleum ether 40-60° C. |
| ether | |
| PyBroP | Bromo-tris-pyrrolidino-phosphonium hexafluorophos- |
| | phate |
| RBF | Round bottomed flask |
| RT or rt | Room temperature |
| SOCl₂ | Thionyl chloride |
| T3P | 2,4,6-Tripropyl-1,3,5,2,4,6-trioxatriphosphorinane- |
| | 2,4,6-trioxide |
| TBME | tert-Butyl methyl ether |
| THF | Tetrahydrofuran |
| TLC | Thin-layer chromatography |

### Naming

The exemplified compounds were named using Dotmatics ELN or Perkin-Elmer ChemDraw software. Other compounds, particularly commercial reagents, either use names generated by Dotmatics ELN or Perkin-Elmer ChemDraw software or names commonly found in online databases and catalogues.

### NMR

All NMR spectra were obtained using Varian VNMRS 600; Varian VNMRS 500; Bruker Avance III 500 or Bruker Avance 400 spectrometers. Chemical shifts are denoted in parts per million (ppm, δ) relative to residual isotopic solvent as described in, for example, Gottlieb et al. J. Org Chem. (1997) 62 7512. The observed multiplicity of certain signals are abbreviated by: s (singlet); br (broad); d (doublet); t (triplet); q (quartet); m (multiplet); or combinations thereof. The number of protons (n) for a given resonance signal is indicated by nH. Coupling constants (J) are designated in Hz and reported to 1 decimal place.

### Mass Spectrometry

Mass spectrometry data were recorded as part of LCMS analysis obtained using a Waters 2695 HPLC coupled to a Thermo LCQ ESI-MS or APCI-MS mass spectrometer; a Shimadzu Prominence Series coupled to a LCMS-2020 ESI and APCI mass spectrometer or Waters Acquity H-class plus UPLC coupled to a Waters Acquity QDa API-ES mass detector. Only molecular ions, fractions from molecular ions and other major peaks are reported as mass/charge (m/z) ratios.

### Example 1 N-(6-Imidazol-1-yl-2-methoxy-3-pyridyl)-5-methyl-3-phenyl-isoxazole-4-carboxamide

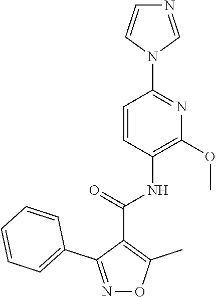 [SMILES: COc1nc(-n2ccnc2)ccc1NC(=O)c1c(-c2ccccc2)noc1C]

### Step 1—Synthesis of Intermediate 1-N-(6-Bromo-2-methoxy-3-pyridyl)-5-methyl-3-phenyl-isoxazole-4-carboxamide

5-Methyl-3-phenyl-1,2-oxazole-4-carboxylic acid (10 g, 49.25 mmol) was suspended in SOCl₂ (39.5 mL, 541.77 mmol). The mixture was split across 4×20 mL Biotage microwave vials and heated to 65° C. overnight using an aluminium heating block. After allowing to cool to room temperature, the vials were combined and excess SOCl₂ was removed under reduced pressure in the fumehood. The residue was dissolved in DCM (10 mL) and added drop-wise to a solution of 6-bromo-2-methoxy-3-pyridinamine (10 g, 49.25 mmol) and Et₃N (8.2 mL, 59.1 mmol) in DCM (50 mL) which was pre-cooled in an ice bath. Upon completion of addition (approximately 10 mins), the cooling bath was removed and the mixture left to stir for 2 h at room temperature. The reaction mixture was diluted with DCM (60 mL) and washed sequentially with water (2×50 mL), 2M NaOH solution (2×40 mL), 1M HCl solution (3×40 mL) and brine (40 mL). The organic extract was dried over MgSO₄, filtered and concentrated under reduced pressure to give N-(6-bromo-2-methoxy-3-pyridyl)-5-methyl-3-phenyl-isoxazole-4-carboxamide (Intermediate 1) (18.14 g, 90% yield) as a brown solid. ¹H NMR (500 MHZ, Chloroform-d) δ8.54 (d, J=8.2 Hz, 1H), 7.65-7.53 (m, 6H), 7.02 (d, J=8.2 Hz, 1H), 3.60 (s, 3H), 2.81 (s, 3H). m/z 390.0 [M+H]⁺ for ⁸¹Br.

### Step 2

A suspension of Intermediate 1 (1.00 g, 2.58 mmol), imidazole (220 mg, 3.23 mmol), L-proline (120 mg, 1.04 mmol) and K₂CO₃ (720 mg, 5.21 mmol) in DMSO (10 mL) was sparged with nitrogen for 10 mins. Copper iodide (200 mg, 1.05 mmol) was added to the mixture which was heated to 80° C. in a sealed 20 mL microwave vial with conventional heating overnight. The mixture was partitioned between 200 mL EtOAc, 200 ml water and 100 mL brine. The mixture was filtered through celite, separated and the aqueous extracted with EtOAc (2×150 mL). The combined organic layers were washed with 100 mL 1:1 water:brine, dried over MgSO₄, filtered and concentrated under reduced pressure. Purification by flash silica column chromatography on an ISCO system (24 g silica, elution with a 0-5% MeOH/DCM gradient) gave approximately 800 mg of an orange solid. This was re-dissolved in refluxing isopropanol (10 mL). Approximately 10 ml water was added to induce precipitation. Upon returning to reflux, the solution was hot filtered and cooled to RT at first, then in the freezer at −20° C. for 30 min. Solid was isolated by filtration and washed with 20 mL cold (−20° C.) isopropanol to give N-(6-imidazol-1-yl-2-methoxy-3-pyridyl)-5-methyl-3-phenyl-isoxazole-4-carboxamide (550 mg, 56% yield). ¹H NMR (500 MHZ, DMSO-d₆) δ9.62 (s, 1H), 8.50 (s, 1H), 8.35 (d, J=8.2 Hz, 1H), 7.92 (s, 1H), 7.74-7.69 (m, 2H), 7.60-7.50 (m, 3H), 7.38 (d, J=8.2 Hz, 1H), 7.11 (s, 1H), 3.92 (s, 3H), 2.65 (s, 3H). m/z 376.1 [M+H]⁺.

### Example 2 N-(4-Imidazol-1-yl-2-methoxy-phenyl)-5-methyl-3-phenyl-isoxazole-4-carboxamide

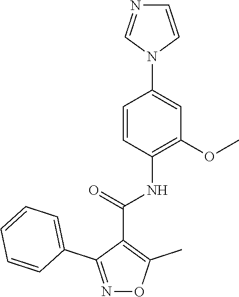 [SMILES: COc1cc(-n2ccnc2)ccc1NC(=O)c1c(-c2ccccc2)noc1C]

### Step 1

To a solution of 4-fluoro-2-methoxy-1-nitro-benzene (1.00 g, 5.8 mmol) in DMF (20 mL) was added K₂CO₃ (808 mg, 5.8 mmol) and imidazole (480 mg, 7.0 mmol). The reaction was stirred at rt for 16 h. The mixture was poured into water (15 mL) and the resultant solid isolated by filtration to give 1-(3-methoxy-4-nitro-phenyl) imidazole (600 mg, 45% yield). ¹H NMR (500 MHZ, Chloroform-d) δ8.03 (d, J=8.8 Hz, 1H), 7.93 (s, 1H), 7.33 (s, 1H), 7.26 (s, 1H), 7.07 (d, J=6.8 Hz, 2H), 4.04 (s, 3H). m/z 220.1 [M+H]⁺.

### Step 2

A solution of 1-(3-methoxy-4-nitro-phenyl) imidazole (350 mg, 1.6 mmol) in MeOH was degassed by bubbling nitrogen gas for 3 min. Palladium on carbon (34 mg, 0.03 mmol) was added, the atmosphere replaced with hydrogen and reaction stirred under H₂ (balloon) for 2 h. TLC indicated the reaction was complete. The reaction mixture was filtered through celite and the clear filtrate was concentrated under reduced pressure to give 4-imidazol-1-yl-2-methoxy-aniline (230 mg, 72% yield) as a light pink solid. ¹H NMR (500 MHZ, Chloroform-d) δ7.72 (s, 1H), 7.16 (dt, J=10.0, 1.2 Hz, 2H), 6.81-6.76 (m, 2H), 6.73 (d, J=8.1 Hz, 1H), 3.91 (s, 2H), 3.88 (s, 3H). m/z 190.1 [M+H]⁺.

### Step 3

To solution of 5-methyl-3-phenyl-1,2-oxazole-4-carboxylic acid (107.4 mg, 0.53 mmol) in DMF (5 mL) were added DIPEA (0.52 mL, 3 mmol) and HATU (301 mg, 0.79 mmol). The reaction mixture was stirred at rt for 10 min before adding 4-imidazol-1-yl-2-methoxy-aniline (100 mg, 0.53 mmol). The reaction mixture was then stirred at rt overnight. The reaction mixture was concentrated to dryness. Water (10 mL) was added and the reaction mixture extracted with EtOAc (3×10 mL). The combined organics were washed with brine, dried over MgSO₄ and filtered before concentration under reduced pressure. Purification by flash silica column chromatography on an ISCO system (elution with 2% MeOH/DCM gradient) to yield N-(4-imidazol-1-yl-2-methoxy-phenyl)-5-methyl-3-phenyl-isoxazole-4-carboxamide (95 mg, 46% yield) as a yellow solid. ¹H NMR (500 MHZ, Chloroform-d) δ8.56 (d, J=8.7 Hz, 1H), 7.82 (s, 2H), 7.67-7.52 (m, 5H), 7.29-7.13 (m, 2H), 6.99-6.91 (m, 1H), 6.82-6.70 (m, 1H), 3.51 (s, 3H), 2.82 (s, 3H). m/z 375.1 [M+H]⁺.

### Example 3 N-(2-Imidazol-1-yl-4-methoxy-pyrimidin-5-yl)-5-methyl-3-phenyl-isoxazole-4-carboxamide

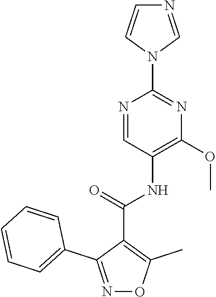 [SMILES: COc1nc(-n2ccnc2)ncc1NC(=O)c1c(-c2ccccc2)noc1C]

### Step 1—Synthesis of Intermediate 2-Imidazol-1-yl-(5-methyl-3-phenyl-isoxazol-4-yl) methanone

To a solution of 5-methyl-3-phenyl-1,2-oxazole-4-carboxylic acid (2.00 g, 9.84 mmol) in THF (25 mL) was added 1,1′-carbonyldiimidazole (1.60 g, 9.84 mmol) portion-wise. Following completion of addition, the reaction mixture was stirred at room temperature overnight. The reaction mixture was concentrated under reduced pressure and the residue dissolved in EtOAc (30 mL), washed with saturated NaHCO₃ (30 mL), water (30 mL) and brine (30 mL), dried over MgSO₄, filtered and concentrated under reduced pressure to give imidazol-1-yl-(5-methyl-3-phenyl-isoxazol-4-yl) methanone (Intermediate 2) (1.92 g, 73%) as an off-white solid. ¹H NMR (500 MHZ, Chloroform-d): δ7.78 (t, J=1.1 Hz, 1H), 7.46-7.49 (m, 2H), 7.39-7.44 (m, 1H), 7.34-7.38 (m, 2H), 7.29 (t, J=1.5 Hz, 1H), 6.95 (dd, J=1.7, 0.8 Hz, 1H), 2.63 (s, 3H). m/z 254.0 [M+H]⁺.

### Step 2—Synthesis of Intermediate 3-N-(2-Chloro-4-methoxy-pyrimidin-5-yl)-5-methyl-3-phenyl-isoxazole-4-carboxamide

To solution of 2-chloro-4-methoxy-pyrimidin-5-amine (1.00 g, 6.27 mmol) in THF (10 mL) was slowly added 1 M lithium bis(trimethylsilyl)amide solution in THF (9.4 mL, 9.4 mmol) at 0° C. The reaction mixture was stirred at 0° C. for 40 min then allowed to warm to room temperature for 10 min. The reaction mixture was then cooled back to 0° C. before adding a solution of Intermediate 2 (1.90 g, 7.52 mmol) in THF (50 mL). The reaction mixture was stirred at 0° C. for 10 min and then allowed to stir at room temperature overnight. The reaction mixture was concentrated to dryness. The residue was taken up in EtOAc (100 mL) and the organics were washed with water (2×15 mL) followed by saturated brine solution (1×25 mL). The organics were dried (MgSO₄) and concentrated under reduced pressure. The crude residue was triturated with MeOH (20 mL) to give N-(2-chloro-4-methoxy-pyrimidin-5-yl)-5-methyl-3-phenyl-isoxazole-4-carboxamide (Intermediate 3). ¹H NMR (600 MHZ, Chloroform-d) δ9.38 (s, 1H), 7.65-7.61 (m, 1H), 7.60-7.57 (m, 4H), 7.41 (s, 1H), 3.70 (s, 3H), 2.82 (s, 3H). m/z 345.1 [M+H]⁺.

### Step 3

To a solution of Intermediate 3 (500 mg, 1.45 mmol) in DMSO (12 mL) were added imidazole (198 mg, 2.9 mmol) and K₂CO₃ (601 mg, 4.35 mmol). The reaction mixture was then heated to 100° C. o/n. The reaction mixture was concentrated to dryness. The residue was taken up in EtOAc (25 mL) and the organics were washed with water (2×5 mL) then saturated brine solution (1×20 mL). The organics were dried (MgSO₄) and concentrated under reduced pressure. The crude material was purified by flash silica column chromatography on an ISCO system (1-2% MeOH/DCM gradient) to give N-(2-imidazol-1-yl-4-methoxy-pyrimidin-5-yl)-5-methyl-3-phenyl-isoxazole-4-carboxamide (123 mg, 21% yield). ¹H NMR (600 MHz, Chloroform-d) δ9.43 (s, 1H), 8.46 (t, J=1.0 Hz, 1H), 7.77 (d, J=1.5 Hz, 1H), 7.68-7.49 (m, 5H), 7.44 (s, 1H), 7.10 (d, J=1.1 Hz, 1H), 3.76 (s, 3H), 2.84 (s, 3H). m/z 377.1 [M+H]⁺.

### Example 4 N-[2-Methoxy-6-(1-methylpyrazol-4-yl)-3-pyridyl]-5-methyl-3-phenyl-isoxazole-4-carboxamide

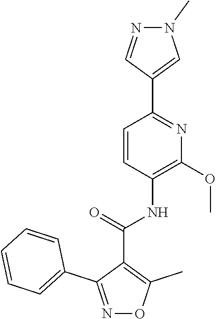 [SMILES: COc1nc(-c2cnn(C)c2)ccc1NC(=O)c1c(-c2ccccc2)noc1C]

A solution of Intermediate 1 (60 mg, 0.15 mmol) and potassium phosphate tribasic (66 mg, 0.31 mmol) in 1,4-dioxane (2 mL) was treated with a solution of 1-methyl-1H-pyrazole-4-boronic acid (23 mg, 0.19 mmol) and the mixture sparged with nitrogen for 10 mins. The reaction mixture was then charged with Pd(dppf)Cl₂ (complexed with DCM, 13 mg, 0.02 mmol), sealed in a microwave tube and heated at 110° C. for 3 h. The reaction mixture was reduced under reduced pressure and taken up in DCM. Water was added and organics separated using a phase separator. The organics were removed under reduced pressure to yield an oil which was purified by flash silica column chromatography on an ISCO system (0-80% EtOAc/PE gradient) to give N-[2-methoxy-6-(1-methylpyrazol-4-yl)-3-pyridyl]-5-methyl-3-phenyl-isoxazole-4-carboxamide (36 mg, 57% yield). ¹H NMR (600 MHZ, Chloroform-d) δ8.60 (d, J=8.1 Hz, 1H), 7.85 (s, 1H), 7.75 (s, 1H), 7.68 (s, 1H), 7.66-7.58 (m, 3H), 7.56 (dd, J=8.1, 6.6 Hz, 2H), 7.00 (d, J=8.1 Hz, 1H), 3.92 (s, 3H), 3.64 (s, 3H), 2.82 (s, 3H). m/z 390.0 [M+H]⁺.

### Example 5 N-(4-(1H-Imidazol-1-yl)-2-methoxyphenyl)-4-methyl-1-phenyl-1H-1,2,3-triazole-5-carboxamide

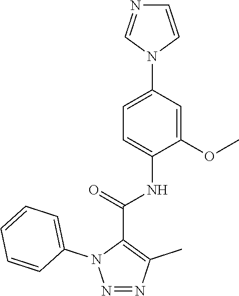 [SMILES: COc1cc(-n2ccnc2)ccc1NC(=O)c1c(C)nnn1-c1ccccc1]

### Step 1

A 100 mL RBF was charged with 5-fluoro-2-nitroanisole (10 g, 58.44 mmol), imidazole (4.77 g, 70.12 mmol), K₂CO₃ (8.08 g, 58.44 mmol) and DMF (50 mL). The reaction mixture was stirred at rt for 3 days. The reaction mixture was treated with water (30 mL) and stirred for 30 mins. The suspension was filtered under reduced pressure, the solid cake washed with water and then air dried to afford 1-(3-methoxy-4-nitrophenyl)-1H-imidazole (10.1 g, 75% yield) as a tan solid. ¹H NMR (500 MHZ, DMSO-d₆) δ8.48 (s, 1H), 8.05 (dd, J=8.8, 1.7 Hz, 1H), 7.94 (s, 1H), 7.56 (d, J=2.1 Hz, 1H), 7.41 (dt, J=8.9, 2.0 Hz, 1H), 7.15 (s, 1H), 4.01 (s, 3H). m/z 219.9 [M+H]⁺.

### Step 2

A 100 mL RBF was charged with 1-(3-methoxy-4-nitrophenyl)-1H-imidazole (1.33 g, 6.07 mmol), palladium on carbon (32.3 mg, 5% mmol) and EtOH (30 mL) under nitrogen. The reaction flask was then purged with H₂ (balloon) and stirred under H₂ at rt overnight. The reaction flask was purged with nitrogen before filtering the reaction mixture through celite, washing with MeOH followed by DCM. The filtrate was then concentrated under reduced pressure to afford 4-(1H-imidazol-1-yl)-2-methoxyaniline (1.05 g, 87% yield) as a brown oil. ¹H NMR (500 MHZ, DMSO-d₆): δ8.01 (s, 1H), 7.53 (s, 1H), 7.00 (d, 2H), 6.87 (d, J=8.3, 1.7 Hz, 1H), 6.67 (d, J=8.3, 1.3 Hz, 1H), 4.87 (s, 2H), 3.81 (s, 3H). m/z 189.9 [M+H]⁺.

### Step 3

A 10 mL microwave vial was charged with 4-(1H-imidazol-1-yl)-2-methoxyaniline (100 mg, 0.53 mmol), 4-methyl-1-phenyl-1H-1,2,3-triazole-5-carboxylic acid (112.8 mg, 0.55 mmol), HATU (221.1 mg, 0.58 mmol), DIPEA (0.20 mL, 1.16 mmol) and DMF (2 mL). The reaction mixture was stirred at rt overnight. The reaction mixture was partitioned between EtOAc (50 mL) and water (50 mL), the phases shaken and the organic layer separated. The organic layer was further washed with brine (30 mL), dried over MgSO₄, filtered and concentrated under reduced pressure. The crude residue was dissolved in DMSO (1 mL) and purified on a Shimadzu MDAP preparative system running in positive mode. Fractions containing product were combined and concentrated under reduced pressure to afford N-(4-(1H-imidazol-1-yl)-2-methoxyphenyl)-4-methyl-1-phenyl-1H-1,2,3-triazole-5-carboxamide (60 mg, 29% yield) as an off-white solid. ¹H NMR (600 MHZ, Methanol-d₄): δ8.27 (s, 1H), 8.14 (d, J=8.5 Hz, 1H), 7.64-7.56 (m, 6H), 7.23 (s, 1H), 7.19 (s, 1H), 7.16 (d, J=8.6 Hz, 1H), 3.87 (s, 3H), 2.58 (s, 3H). m/z 375.0 [M+H]⁺.

### Example 6 N-[6-(3,5-Dimethylisoxazol-4-yl)-2-methoxy-3-pyridyl]-5-methyl-3-phenyl-isoxazole-4-carboxamide

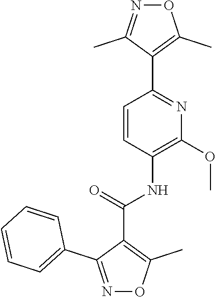 [SMILES: COc1nc(-c2c(C)noc2C)ccc1NC(=O)c1c(-c2ccccc2)noc1C]

Intermediate 1 (50 mg, 0.13 mmol), potassium phosphate tribasic (55 mg, 0.26 mmol) and (3,5-dimethylisoxazol-4-yl) boronic acid (28 mg, 0.15 mmol) were added to a microwave vial, backfilled with nitrogen and 1,4-dioxane (2 mL) added. The mixture was purged with nitrogen (10 mins) before Pd(dppf)Cl₂ (complexed with DCM, 11 mg, 0.01 mmol) was added and vial sealed. The mixture was purged for a further 10 mins then reacted in a microwave reactor for 2 h at 110° C. Solvent was removed via Genevac and to the resulting solid was added DCM and water. The bi-phasic solution was stirred vigorously before being passed through a phase separator. The filtrate was concentrated under reduced pressure and the residue purified by flash silica column chromatography on an ISCO system to give N-[6-(3,5-dimethylisoxazol-4-yl)-2-methoxy-3-pyridyl]-5-methyl-3-phenyl-isoxazole-4-carboxamide (11 mg, 20% yield). ¹H NMR (600 MHZ, Chloroform-d) δ8.69 (d, J=8.1 Hz, 1H), 7.72 (s, 1H), 7.66-7.60 (m, 3H), 7.60-7.55 (m, 2H), 6.91 (d, J=8.1 Hz, 1H), 3.63 (s, 3H), 2.83 (s, 3H), 2.56 (s, 3H), 2.42 (s, 3H). m/z 403.0 [M−H]—.

### Example 7 N-(2-Methoxy-6-pyrimidin-5-yl-3-pyridyl)-5-methyl-3-phenyl-isoxazole-4-carboxamide

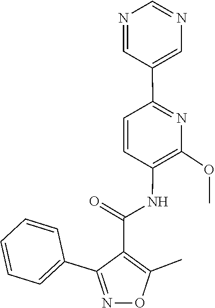 [SMILES: COc1nc(-c2cncnc2)ccc1NC(=O)c1c(-c2ccccc2)noc1C]

A solution of Intermediate 1 (500 mg, 1.29 mmol) and pyrimidine-5-boronic acid (191.5 mg, 1.55 mmol) in 1,4-dioxane (20 mL) and water (2 mL) was treated with KOAc (505.5 mg, 5.15 mmol) and the reaction mixture purged with nitrogen for 10 mins. The reaction mixture was then treated with Pd-118 (84 mg, 0.13 mmol) sealed and heated at 80° C. for 3 h, then at room temperature overnight. The reaction mixture was concentrated under reduced pressure to give a brown solid. The residue was partitioned between EtOAc (150 mL) and water (150 mL), the phases shaken and separated. The organic phase was washed with brine (50 mL), dried (MgSO₄), filtered and concentrated under reduced pressure to give a yellow solid. The residue was purified by flash silica column chromatography on an ISCO system (40 g silica, elution with a 0-20% EtOAc/PE gradient) using collect all function to give N-(2-methoxy-6-pyrimidin-5-yl-3-pyridyl)-5-methyl-3-phenyl-isoxazole-4-carboxamide (382 mg, 73% yield). ¹H NMR (600 MHZ, Chloroform-d) δ9.25 (s, 2H), 9.17 (s, 1H), 8.77 (d, J=8.1 Hz, 1H), 7.79 (s, 1H), 7.66-7.61 (m, 3H), 7.58 (ddd, J=7.7, 6.5, 2.1 Hz, 2H), 7.37 (d, J=8.1 Hz, 1H), 3.70 (s, 3H), 2.83 (s, 3H). m/z 388.0 [M+H]⁺

### Example 8 N-[2-Methoxy-6-(3-nitrophenyl)-3-pyridyl]-5-methyl-3-phenyl-isoxazole-4-carboxamide

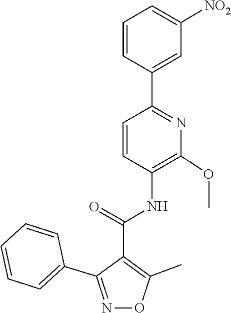 [SMILES: COc1nc(-c2cccc([N+](=O)[O-])c2)ccc1NC(=O)c1c(-c2ccccc2)noc1C]

Intermediate 1 (50 mg, 0.13 mmol), potassium phosphate tribasic (55 mg, 0.26 mmol) and (3-nitrophenyl) boronic acid (28 mg, 0.15 mmol) were added to a microwave vial, backfilled with nitrogen and 1,4-dioxane (2 mL) added. The mixture was purged with nitrogen (10 min) before Pd(dppf)Cl₂ (complexed with DCM, 11 mg, 0.01 mmol) was added and the vial sealed. The reaction mixture was purged for a further 10 mins then reacted in a microwave reactor for 2 h at 110° C. Solvent was removed via Genevac and to the resulting solids was added DCM and water. The bi-phasic solution was stirred vigorously before being passed through a phase separator. The filtrate was concentrated under reduced pressure and the residue purified by flash silica column chromatography on an ISCO system to give N-[2-methoxy-6-(3-nitrophenyl)-3-pyridyl]-5-methyl-3-phenyl-isoxazole-4-carboxamide (45 mg, 77% yield). ¹H NMR (600 MHZ, Chloroform-d) δ8.83-8.78 (m, 1H), 8.77 (d, J=8.1 Hz, 1H), 8.28-8.24 (m, 1H), 8.17 (ddd, J=8.1, 2.3, 1.0 Hz, 1H), 7.80 (s, 1H), 7.68-7.62 (m, 3H), 7.62-7.54 (m, 3H), 7.42 (d, J=8.1 Hz, 1H), 3.73 (s, 3H), 2.84 (s, 3H). m/z 429.0 [M+H]⁺.

### Example 9 N-(3-Methoxy-5-pyrimidin-5-yl-2-pyridyl)-5-methyl-3-phenyl-isoxazole-4-carboxamide

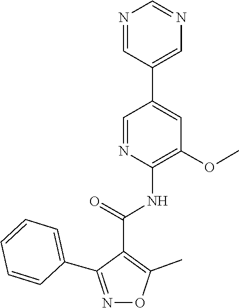 [SMILES: COc1cc(-c2cncnc2)cnc1NC(=O)c1c(-c2ccccc2)noc1C]

### Step 1—Synthesis of Intermediate 4-N-(5-Bromo-3-methoxy-2-pyridyl)-5-methyl-3-phenyl-isoxazole-4-carboxamide

To solution of 5-bromo-3-methoxypyridin-2-amine (500 mg, 2.46 mmol) in DMF (15 mL) was added Intermediate 2 (623 mg, 2.46 mmol). The reaction mixture was heated at 80° C. o/n. Heating was then increased to 120° C. for a further 48 h. The reaction mixture was concentrated to dryness and the residue purified by flash silica column chromatography (gradient 25-40% EtOAc/PE) to give N-(5-bromo-3-methoxy-2-pyridyl)-5-methyl-3-phenyl-isoxazole-4-carboxamide (Intermediate 4) as a white solid (345 mg, 35% yield). ¹H NMR (600 MHZ, Chloroform-d) δ8.06 (d, J=2.0 Hz, 1H), 7.80 (s, 1H), 7.70-7.58 (m, 2H), 7.57-7.47 (m, 3H), 7.08 (d, J=1.9 Hz, 1H), 3.52 (s, 3H), 2.81 (s, 3H). m/z 389.9 [M+H]⁺.

### Step 2

A solution of potassium phosphate tribasic (54.6 mg, 0.26 mmol), pyrimidine-5-boronic acid (19.1 mg, 0.15 mmol) and Intermediate 4 (50 mg, 0.13 mmol) in 1,4-dioxane (2 mL) and water (0.2 mL) was degassed with nitrogen for 15 min. Pd-118 (4.2 mg, 0.01 mmol) was quickly added, the solution degassed again for 10 min and the mixture left to stir at 100° C. overnight. Water (10 mL) was added and the reaction mixture extracted with DCM (3×10 mL). The combined organics were washed with brine, dried over MgSO₄ and filtered before concentration to dryness. Purification by flash silica column chromatography on an ISCO system (elution with 3% MeOH/DCM gradient) to give N-(3-methoxy-5-pyrimidin-5-yl-2-pyridyl)-5-methyl-3-phenyl-isoxazole-4-carboxamide (32 mg, 61% yield) as a white solid. ¹H NMR (600 MHZ, Chloroform-d) δ9.23 (s, 1H), 8.88 (s, 2H), 8.24 (d, J=2.0 Hz, 1H), 8.00 (s, 1H), 7.65 (d, J=7.4 Hz, 2H), 7.58 (t, J=7.3 Hz, 1H), 7.54 (t, J=7.4 Hz, 2H), 7.10 (d, J=2.1 Hz, 1H), 3.60 (s, 3H), 2.84 (s, 3H). m/z 388.0 [M+H]⁺.

### Example 10 N-[2-Methoxy-6-(2-methoxythiazol-5-yl)-3-pyridyl]-5-methyl-3-phenyl-isoxazole-4-carboxamide

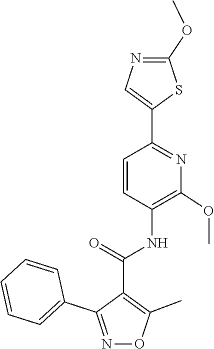 [SMILES: COc1ncc(-c2ccc(NC(=O)c3c(-c4ccccc4)noc3C)c(OC)n2)s1]

A solution of Intermediate 1 (60 mg, 0.15 mmol) and 2-methoxy-5-(tetramethyl-1,3,2-dioxaborolan-2-yl)-1,3-thiazole (45 mg, 0.19 mmol) in 1,4-dioxane (2 mL) and water (0.20 mL) was treated with potassium phosphate tribasic (98 mg, 0.46 mmol) and the mixture sparged with nitrogen for 10 mins. The reaction mixture was then charged with Pd(dppf)Cl₂ (complexed with DCM, 13 mg, 0.02 mmol) sealed and heated at 110° C. overnight. The reaction mixture was concentrated under reduced pressure and taken up in DCM (10 mL). Water (10 mL) was added and the organics separated using a phase separator. The organics were concentrated under reduced pressure and the residue purified by flash silica column chromatography on an ISCO system (12 g silica, elution with a 0-40% EtOAc/PE gradient) to give N-[2-methoxy-6-(2-methoxythiazol-5-yl)-3-pyridyl]-5-methyl-3-phenyl-isoxazole-4-carboxamide (15 mg, 21% yield). ¹H NMR (600 MHZ, Chloroform-d) δ8.63 (d, J=8.1 Hz, 1H), 7.68 (s, 1H), 7.64-7.59 (m, 3H), 7.59-7.52 (m, 2H), 7.48 (s, 1H), 7.08 (d, J=8.2 Hz, 1H), 4.08 (s, 3H), 3.62 (s, 3H), 2.82 (s, 3H). m/z 423.0 [M+H]⁺.

### Example 11 N-[6-(5-Amino-3-pyridyl)-2-methoxy-3-pyridyl]-5-methyl-3-phenyl-isoxazole-4-carboxamide

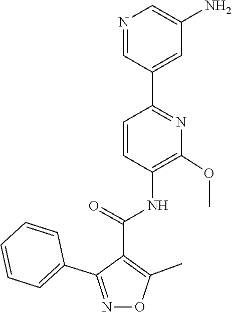 [SMILES: COc1nc(-c2cncc(N)c2)ccc1NC(=O)c1c(-c2ccccc2)noc1C]

A solution of Intermediate 1 (50 mg, 0.13 mmol) and 5-(4,4,5,5-tetramethyl-1,3,2-dioxaborolan-2-yl)pyridin-3-amine (34 mg, 0.15 mmol) in 1,4-dioxane (2 mL) and water (0.20 mL) was treated with KOAc (51 mg, 0.52 mmol) and the mixture sparged with nitrogen for 10 mins. The reaction mixture was then charged with Pd-118 (8 mg, 0.01 mmol), sealed and heated at 80° C. overnight. The reaction mixture was concentrated under reduced pressure and taken up in DCM (10 mL). Water (10 mL) was added and the organics separated using a phase separator. The organics were concentrated under reduced pressure to yield a dark oil which was purified by flash silica column chromatography on an ISCO system (12 g silica, elution with a 0-50% EtOAc/PE gradient) to give N-[6-(5-amino-3-pyridyl)-2-methoxy-3-pyridyl]-5-methyl-3-phenyl-isoxazole-4-carboxamide (43 mg, 79% yield). ¹H NMR (600 MHZ, Chloroform-d) δ8.71 (d, J=8.2 Hz, 1H), 8.59 (d, J=1.8 Hz, 1H), 8.06 (d, J=2.7 Hz, 1H), 7.76 (s, 1H), 7.63 (tt, J=8.7, 1.5 Hz, 3H), 7.61-7.55 (m, 2H), 7.53 (dd, J=2.7, 1.8 Hz, 1H), 7.32 (d, J=8.2 Hz, 1H), 3.75 (s, 2H), 3.70 (s, 3H), 2.84 (s, 3H). m/z 402.0 [M+H]⁺.

### Example 12 N-[2-Methoxy-4-(1-methylpyrazol-4-yl)phenyl]-5-methyl-3-phenyl-isoxazole-4-carboxamide

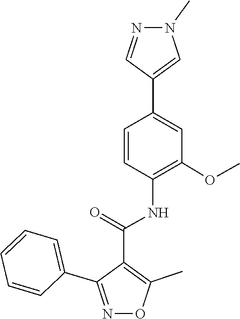 [SMILES: COc1cc(-c2cnn(C)c2)ccc1NC(=O)c1c(-c2ccccc2)noc1C]

### Step 1—Synthesis of Intermediate 5-N-(4-Bromo-2-methoxy-phenyl)-5-methyl-3-phenyl-isoxazole-4-carboxamide

To a mixture of 4-bromo-o-anisidine (1.00 g, 4.95 mmol), DIPEA (1.72 mL, 9.9 mmol) in DCM (40 mL) was added dropwise a 0.4 M solution of 5-methyl-3-phenyl-isoxazole-4-carbonyl chloride (12.37 mL, 4.95 mmol) in DCM (synthesised according to Step 1, Example 1). The reaction mixture was stirred overnight at rt. Water (20 mL) was added and the reaction mixture extracted with DCM (3×20 mL). The combined organics were washed with brine, dried over MgSO₄ and filtered before concentrating to dryness. Purification by flash silica column chromatography on an ISCO system (elution with 20% EtOAc/PE gradient) to yield N-(4-bromo-2-methoxy-phenyl)-5-methyl-3-phenyl-isoxazole-4-carboxamide (Intermediate 5) (1.50 g, 74% yield). ¹H NMR (400 MHZ, Chloroform-d) δ8.34 (d, J=8.7 Hz, 1H), 7.74 (s, 1H), 7.67-7.49 (m, 5H), 7.06 (dd, J=8.7, 2.1 Hz, 1H), 6.83 (d, J=2.1 Hz, 1H), 3.43 (s, 3H), 2.80 (s, 3H). m/z 387.0/389.0 [M+H]⁺.

### Step 2

A 5 mL microwave vial was charged with Intermediate 5 (50 mg, 0.13 mmol), 1-methyl-1H-pyrazole-4-boronic acid (24 mg, 0.19 mmol), Na₂CO₃ (27 mg, 0.26 mmol), tetrakis(triphenylphosphine) palladium (0) (15 mg, 0.01 mmol), ethylene glycol dimethyl ether (2 mL) and water (1 mL). The vial was sealed, flushed with nitrogen and degassed before heated at 100° C. in a microwave for 45 min. The reaction mixture was partitioned between water (20 mL) and EtOAc (20 mL) and separated. The organic layer was washed with brine (20 mL), dried over MgSO₄, filtered through celite and concentrated under reduced pressure. The residue was purified on a Shimadzu MDAP (eluting with 30-95% MeCN/water+0.1% formic acid gradient) to afford N-[2-methoxy-4-(1-methylpyrazol-4-yl)phenyl]-5-methyl-3-phenyl-isoxazole-4-carboxamide (16 mg, 31% yield) as an off-white solid. ¹H NMR (600 MHZ, Chloroform-d) δ8.42 (d, J=8.4 Hz, 1H), 7.81 (s, 1H), 7.68 (s, 1H), 7.65 (d, J=7.6 Hz, 2H), 7.60-7.51 (m, 4H), 7.04 (d, J=8.4 Hz, 1H), 6.80 (d, J=1.7 Hz, 1H), 3.93 (s, 3H), 3.49 (s, 3H), 2.82 (s, 3H). m/z 389.0 [M+H]⁺.

### Example 13 N-(2-Methoxy-4-pyrimidin-5-yl-phenyl)-5-methyl-3-phenyl-isoxazole-4-carboxamide

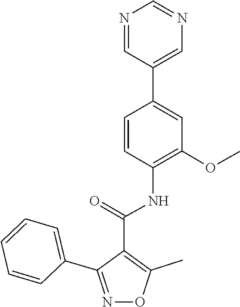 [SMILES: COc1cc(-c2cncnc2)ccc1NC(=O)c1c(-c2ccccc2)noc1C]

A 5 mL microwave vial was charged with Intermediate 5 (50 mg, 0.13 mmol), pyrimidine-5-boronic acid (19 mg, 0.15 mmol), Na₂CO₃ (27 mg, 0.26 mmol), tetrakis(triphenylphosphine) palladium (0) (15 mg, 0.01 mmol), ethylene glycol dimethyl ether (2 mL) and water (1 mL). The vial was sealed, flushed with nitrogen and degassed before heated at 90° C. in a microwave for 4 h. The reaction mixture was partitioned between EtOAc (20 mL) and water (20 mL) and the phases were separated. The organic phase was washed with brine (20 mL), dried over MgSO₄, filtered through celite and concentrated under reduced pressure. The residue was adsorbed onto silica and purified by flash column chromatography (4 g silica, elution with a 0-80% EtOAc/PE gradient) to afford N-(2-methoxy-4-pyrimidin-5-yl-phenyl)-5-methyl-3-phenyl-isoxazole-4-carboxamide (28 mg, 53% yield) as a yellow solid. ¹H NMR (600 MHz, Chloroform-d) δ9.17 (s, 1H), 8.89 (s, 2H), 8.62 (d, J=8.4 Hz, 1H), 7.90 (s, 1H), 7.67-7.63 (m, 2H), 7.61 (t, J=7.3 Hz, 1H), 7.56 (t, J=7.4 Hz, 2H), 7.19-7.15 (m, 1H), 6.89 (d, J=1.9 Hz, 1H), 3.53 (s, 3H), 2.84 (s, 3H). m/z 387.0 [M+H]⁺.

### Example 14 N-[5-(3,5-Dimethylisoxazol-4-yl)-3-methoxy-2-pyridyl]-5-methyl-3-phenyl-isoxazole-4-carboxamide

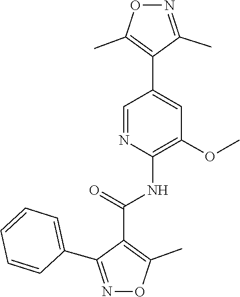 [SMILES: COc1cc(-c2c(C)noc2C)cnc1NC(=O)c1c(-c2ccccc2)noc1C]

A solution of KOAc (38 mg, 0.39 mmol), 3,5-dimethylisoxazole-4-boronic acid (21.8 mg, 0.15 mmol) and Intermediate 4 (50 mg, 0.13 mmol) in 1,4-dioxane (2 mL) and water (0.2 mL) was degassed with nitrogen for 15 min. Pd-118 (4.2 mg, 0.01 mmol) was quickly added, the solution degassed again for 10 min and the mixture left to stir at 80° C. overnight. Water (10 mL) was added and the reaction mixture extracted with DCM (3×10 mL). The combined organics were washed with brine, dried over MgSO₄ and filtered before concentrating to dryness. Purification by flash silica column chromatography gave N-[5-(3,5-dimethylisoxazol-4-yl)-3-methoxy-2-pyridyl]-5-methyl-3-phenyl-isoxazole-4-carboxamide (26 mg, 47% yield) as a light yellow solid. ¹H NMR (600 MHZ, Chloroform-d) δ8.01-7.91 (m, 2H), 7.74-7.63 (m, 2H), 7.55 (dt, J=14.7, 7.2 Hz, 3H), 6.83 (d, J=1.9 Hz, 1H), 3.55 (s, 3H), 2.84 (s, 3H), 2.38 (s, 3H), 2.23 (s, 3H). m/z 405.0 [M+H]⁺.

### Example 15 N-[3-Methoxy-5-(1-methylpyrazol-4-yl)-2-pyridyl]-5-methyl-3-phenyl-isoxazole-4-carboxamide

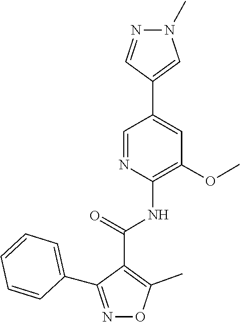 [SMILES: COc1cc(-c2cnn(C)c2)cnc1NC(=O)c1c(-c2ccccc2)noc1C]

A solution of KOAc (38 mg, 0.39 mmol), 1-methyl-1H-pyrazole-4-boronic acid (19.4 mg, 0.15 mmol) and Intermediate 4 (50 mg, 0.13 mmol) in 1,4-dioxane (2 mL) and water (0.2 mL) was degassed with nitrogen for 15 min. Pd-118 (4.2 mg, 0.01 mmol) was quickly added, the solution degassed again for 10 min and the mixture left to stir at 80° C. overnight. Water (10 mL) was added and the reaction mixture extracted with DCM (3×10 mL). The combined organics were washed with brine, dried over MgSO₄ and filtered before concentrating to dryness. Purification by flash silica column chromatography gave N-[3-methoxy-5-(1-methylpyrazol-4-yl)-2-pyridyl]-5-methyl-3-phenyl-isoxazole-4-carboxamide (22 mg, 42% yield) as a white solid. ¹H NMR (600 MHZ, Chloroform-d) δ8.14 (s, 1H), 7.83 (s, 1H), 7.67 (s, 1H), 7.65 (d, J=7.4 Hz, 2H), 7.59 (s, 1H), 7.55-7.53 (m, 2H), 7.51 (d, J=7.4 Hz, 1H), 7.01 (s, 1H), 3.94 (s, 3H), 3.57 (s, 3H), 2.82 (s, 3H). m/z 390.4 [M+H]⁺.

### Example 16 N-[6-[3-(Dimethylamino)phenyl]-2-methoxy-3-pyridyl]-5-methyl-3-phenyl-isoxazole-4-carboxamide

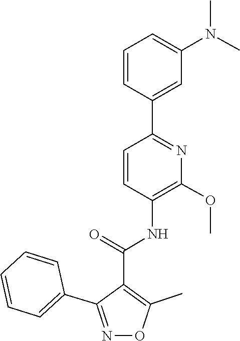 [SMILES: COc1nc(-c2cccc(N(C)C)c2)ccc1NC(=O)c1c(-c2ccccc2)noc1C]

A solution of Intermediate 1 (50 mg, 0.13 mmol) and [3-(dimethylamino)phenyl] boronic acid (26 mg, 0.15 mmol) in 1,4-dioxane (2 mL) was treated with KOAc (51 mg, 0.52 mmol) and the mixture sparged with nitrogen for 10 mins. The reaction was then charged with Pd-118 (8 mg, 0.01 mmol), sealed and heated at 80° C. overnight. The reaction mixture was concentrated via Genevac then DCM and water added. The mixture was vigorously agitated before being passed through a phase separator. Solvent was evaporated and the residue purified by flash silica column chromatography on an ISCO system (elution with a 0-50% PE/EtOAc gradient) to give N-[6-[3-(dimethylamino)phenyl]-2-methoxy-3-pyridyl]-5-methyl-3-phenyl-isoxazole-4-carboxamide (42 mg, 72% yield). ¹H NMR (600 MHZ, Chloroform-d) δ8.68 (d, J=8.2 Hz, 1H), 7.75 (s, 1H), 7.67-7.54 (m, 5H), 7.38-7.35 (m, 1H), 7.33 (d, J=8.2 Hz, 1H), 7.31-7.27 (m, 2H), 6.78-6.72 (m, 1H), 3.71 (s, 3H), 3.00 (s, 6H), 2.83 (s, 3H). m/z 429.0 [M+H]⁺.

### Example 17 N-[6-(2-Cyclopropylpyrimidin-5-yl)-2-methoxy-3-pyridyl]-5-methyl-3-phenyl-isoxazole-4-carboxamide

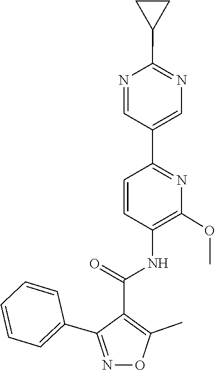 [SMILES: COc1nc(-c2cnc(C3CC3)nc2)ccc1NC(=O)c1c(-c2ccccc2)noc1C]

A solution of Intermediate 1 (50 mg, 0.13 mmol) and (2-cyclopropylpyrimidin-5-yl) boronic acid (25 mg, 0.15 mmol) in 1,4-dioxane (2 mL) was treated with KOAc (51 mg, 0.52 mmol) and the mixture sparged with nitrogen for 10 mins. The reaction mixture was then charged with Pd-118 (8 mg, 0.01 mmol), sealed and heated at 80° C. overnight. The reaction mixture was concentrated via Genevac then DCM and water added. The reaction mixture was vigorously agitated before being passed through a phase separator. Solvent was evaporated and the residue purified by flash silica column chromatography on an ISCO system with further purification by mass directed preparative HPLC to give N-[6-(2-cyclopropylpyrimidin-5-yl)-2-methoxy-3-pyridyl]-5-methyl-3-phenyl-isoxazole-4-carboxamide (8 mg, 16% yield). ¹H NMR (600 MHZ, Chloroform-d) δ9.07 (s, 2H), 8.74 (d, J=8.2 Hz, 1H), 7.77 (s, 1H), 7.66-7.61 (m, 3H), 7.60-7.55 (m, 2H), 7.29 (d, J=8.2 Hz, 1H), 3.68 (d, J=1.3 Hz, 3H), 2.84 (s, 3H), 2.29 (tt, J=8.4, 4.7 Hz, 1H), 1.17 (dt, J=6.2, 3.3 Hz, 2H), 1.13-1.08 (m, 2H). m/z 428.0 [M+H]⁺.

### Example 18 N-[3-Methoxy-5-(1H-pyrazol-4-yl)-2-pyridyl]-5-methyl-3-phenyl-isoxazole-4-carboxamide

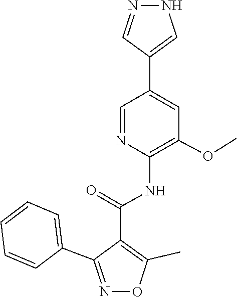 [SMILES: COc1cc(-c2cn[nH]c2)cnc1NC(=O)c1c(-c2ccccc2)noc1C]

A solution of KOAc (25.2 mg, 0.26 mmol), 1H-pyrazole-4-boronic acid pinacol ester (30 mg, 0.15 mmol) and Intermediate 4 (50 mg, 0.13 mmol) in 1,4-dioxane (2 mL) and water (0.2 mL) was degassed with nitrogen for 15 min. Pd-118 (4.2 mg, 0.01 mmol) was quickly added, the solution degassed again for 10 min and the mixture left to stir at 80° C. overnight. Water (10 mL) was added and the reaction mixture extracted with DCM (3×10 mL). The combined organics were washed with brine, dried over MgSO₄ and filtered before concentration to dryness. Purification by flash silica column chromatography gave N-[3-methoxy-5-(1H-pyrazol-4-yl)-2-pyridyl]-5-methyl-3-phenyl-isoxazole-4-carboxamide (23 mg, 45% yield) as a white solid. ¹H NMR (600 MHZ, Chloroform-d) δ10.65 (br s, 1H), 8.19 (s, 1H), 7.88 (s, 1H), 7.82 (s, 2H), 7.65 (d, J=7.3 Hz, 2H), 7.53 (dt, J=15.0, 7.4 Hz, 3H), 7.06 (s, 1H), 3.59 (s, 3H), 2.82 (s, 3H). m/z 375.9 [M+H]⁺.

### Example 19 N-[3-Methoxy-5-(1-methylpyrazol-4-yl) pyrazin-2-yl]-5-methyl-3-phenyl-isoxazole-4-carboxamide

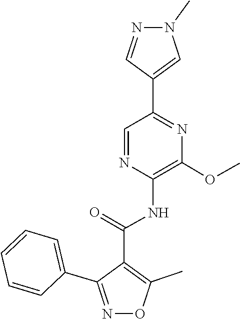 [SMILES: COc1nc(-c2cnn(C)c2)cnc1NC(=O)c1c(-c2ccccc2)noc1C]

### Step 1—Synthesis of Intermediate 6-N-(5-Bromo-3-methoxy-pyrazin-2-yl)-5-methyl-3-phenyl-isoxazole-4-carboxamide

To a solution of 5-bromo-3-methoxypyrazin-2-amine (13.50 g, 66.17 mmol) in anhydrous THF (40 mL) at −78° C., a solution of lithium bis(trimethylsilyl)amide (99.25 mL, 99.25 mmol) was added. The reaction mixture was stirred at −78° C. for 10 mins, allowed to warm to room temperature for 20 mins then cooled back down to −78° C. A solution of Intermediate 2 (20.11 g, 79.4 mmol) in THF (30 mL) was added slowly and the reaction mixture gradually allowed to warm to room temperature and left to stir over the weekend. The reaction mixture was concentrated under reduced pressure to give a brown solid. The brown residue was dissolved in MeOH (250 mL) with the aid of heating/sonication and treated with water (50 mL). The precipitate formed was filtered under reduced pressure, washed with water and air-dried. The solid was dissolved in EtOAc (200 mL) with the aid of heating/sonication and washed with water (100 mL), dried over MgSO₄, filtered and concentrated under reduced pressure to give N-(5-bromo-3-methoxy-pyrazin-2-yl)-5-methyl-3-phenyl-isoxazole-4-carboxamide (Intermediate 6, 13.2 g, 49%) as a tan coloured solid. ¹H NMR (600 MHZ, Chloroform-d) δ8.00 (s, 1H), 7.78 (s, 1H), 7.62-7.57 (m, 3H), 7.57-7.52 (m, 2H), 3.68 (s, 3H), 2.82 (s, 3H). m/z 390.9 [M+H]⁺ for ⁸¹Br.

### Step 2

A 5 mL microwave vial was charged with Intermediate 6 (100 mg, 0.26 mmol), Na₂CO₃ (54 mg, 0.51 mmol), tetrakis(triphenylphosphine) palladium (0) (30 mg, 0.03 mmol), 1-methyl-1H-pyrazole-4-boronic acid (49 mg, 0.39 mmol), ethylene glycol dimethyl ether (2 mL) and water (1 mL). The vial was sealed, flushed with nitrogen and degassed before heated at 120° C. in the microwave for 1 h. The reaction mixture was partitioned between EtOAc (20 mL) and water (20 mL) and the phases were separated. The organic phase was washed with brine (20 mL), dried over MgSO₄, filtered through celite and concentrated under reduced pressure. The residue was adsorbed onto silica and purified by flash column chromatography (4 g silica, elution with a 0-80% EtOAc/PE gradient) to afford N-[3-methoxy-5-(1-methylpyrazol-4-yl) pyrazin-2-yl]-5-methyl-3-phenyl-isoxazole-4-carboxamide (50 mg, 48% yield) as a yellow solid. ¹H NMR (600 MHZ, Chloroform-d) δ8.09 (s, 1H), 7.88 (s, 1H), 7.82 (s, 1H), 7.78 (s, 1H), 7.66-7.62 (m, 2H), 7.59 (t, J=7.4 Hz, 1H), 7.55 (t, J=7.5 Hz, 2H), 3.94 (s, 3H), 3.72 (s, 3H), 2.83 (s, 3H). m/z 391.0 [M+H]⁺.

### Example 20 N-[6-(1,3-Dimethylpyrazol-4-yl)-2-methoxy-3-pyridyl]-5-methyl-3-phenyl-isoxazole-4-carboxamide

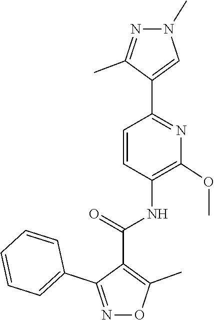 [SMILES: COc1nc(-c2cn(C)nc2C)ccc1NC(=O)c1c(-c2ccccc2)noc1C]

A solution of Intermediate 1 (50 mg, 0.13 mmol) and 1,3-dimethyl-4-(4,4,5,5-tetramethyl-1,3,2-dioxaborolan-2-yl) pyrazole (34 mg, 0.15 mmol) in 1,4-dioxane (2 mL) and water (0.2 mL) was treated with KOAc (51 mg, 0.52 mmol) and the mixture sparged with nitrogen for 10 mins. The reaction mixture was then charged with Pd-118 (8 mg, 0.01 mmol), sealed and heated at 80° C. overnight. The reaction mixture was reduced via Genevac then DCM and water added. The mixture was vigorously agitated before being passed through a phase separator. The solvent was evaporated and the residue purified by flash silica column chromatography on an ISCO system (0-10% MeOH/DCM gradient) with further purification by flash silica column chromatography on an ISCO system (0-100% EtOAc/PE gradient) to give N-[6-(1,3-dimethylpyrazol-4-yl)-2-methoxy-3-pyridyl]-5-methyl-3-phenyl-isoxazole-4-carboxamide (37 mg, 69% yield). ¹H NMR (600 MHZ, Chloroform-d) δ8.60 (d, J=8.1 Hz, 1H), 7.68 (s, 1H), 7.67-7.54 (m, 6H), 6.96 (d, J=8.1 Hz, 1H), 3.84 (s, 3H), 3.64 (s, 3H), 2.82 (s, 3H), 2.51 (s, 3H). m/z 404.0 [M+H]⁺.

### Example 21 N-[6-(1-Isopropylpyrazol-4-yl)-2-methoxy-3-pyridyl]-5-methyl-3-phenyl-isoxazole-4-carboxamide

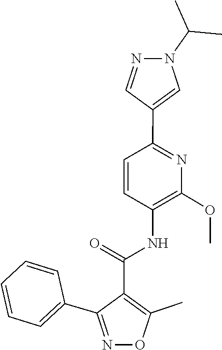 [SMILES: COc1nc(-c2cnn(C(C)C)c2)ccc1NC(=O)c1c(-c2ccccc2)noc1C]

A solution of Intermediate 1 (50 mg, 0.13 mmol) and 1-isopropyl-4-(4,4,5,5-tetramethyl-1,3,2-dioxaborolan-2-yl) pyrazole (37 mg, 0.15 mmol) in 1,4-dioxane (2 mL) and water (0.2 mL) was treated with KOAc (51 mg, 0.52 mmol) and the mixture sparged with nitrogen for 10 mins. The reaction mixture was then charged with Pd-118 (8 mg, 0.01 mmol), sealed and heated at 80° C. overnight. The reaction mixture was reduced via Genevac then DCM and water added. The mixture was vigorously agitated before being passed through a phase separator. Solvent was evaporated and the residue purified by flash silica column chromatography on an ISCO system (0-60% EtOAc/PE gradient) to give N-[6-(1-isopropylpyrazol-4-yl)-2-methoxy-3-pyridyl]-5-methyl-3-phenyl-isoxazole-4-carboxamide (33 mg, 58% yield). ¹H NMR (600 MHZ, Chloroform-d) δ8.60 (d, J=8.1 Hz, 1H), 7.87 (s, 1H), 7.81 (s, 1H), 7.68 (s, 1H), 7.65-7.58 (m, 3H), 7.57-7.54 (m, 2H), 7.02 (d, J=8.1 Hz, 1H), 4.50 (heptet, J=6.7 Hz, 1H), 3.65 (s, 3H), 2.81 (s, 3H), 1.53 (d, J=6.7 Hz, 6H). m/z 418.0 [M+H]⁺.

### Example 22 N-[2-Methoxy-6-(1,3,5-trimethylpyrazol-4-yl)-3-pyridyl]-5-methyl-3-phenyl-isoxazole-4-carboxamide

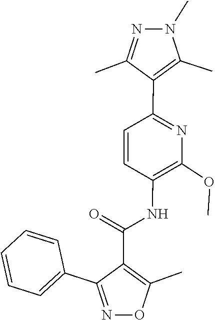 [SMILES: COc1nc(-c2c(C)nn(C)c2C)ccc1NC(=O)c1c(-c2ccccc2)noc1C]

A solution of Intermediate 1 (50 mg, 0.13 mmol) and 1,3,5-trimethyl-4-(4,4,5,5-tetramethyl-1,3,2-dioxaborolan-2-yl) pyrazole (37 mg, 0.15 mmol) in 1,4-dioxane (2 mL) and water (0.2 mL) was treated with KOAc (51 mg, 0.52 mmol) and the mixture sparged with nitrogen for 10 mins. The reaction was then charged with Pd-118 (8 mg, 0.01 mmol), sealed and heated at 80° C. overnight. The reaction mixture was reduced via Genevac then DCM and water added. The mixture was vigorously agitated before being passed through a phase separator. Solvent was evaporated and the residue purified by flash silica column chromatography on an ISCO system (0-90% EtOAc/PE gradient) to give N-[2-methoxy-6-(1,3,5-trimethylpyrazol-4-yl)-3-pyridyl]-5-methyl-3-phenyl-isoxazole-4-carboxamide (18 mg, 32%). ¹H NMR (600 MHZ, Chloroform-d) δ8.64 (d, J=8.1 Hz, 1H), 7.70 (s, 1H), 7.66-7.59 (m, 3H), 7.59-7.55 (m, 2H), 6.86 (d, J=8.1 Hz, 1H), 3.75 (s, 3H), 3.64 (s, 3H), 2.83 (s, 3H), 2.40 (s, 3H), 2.37 (s, 3H). m/z 418.0 [M+H]⁺.

### Example 23 N-[2-Methoxy-6-(2-methylpyrazol-3-yl)-3-pyridyl]-5-methyl-3-phenyl-isoxazole-4-carboxamide

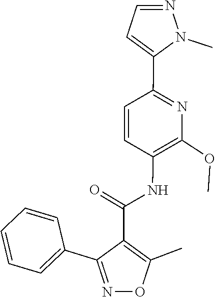 [SMILES: COc1nc(-c2ccnn2C)ccc1NC(=O)c1c(-c2ccccc2)noc1C]

A solution of Intermediate 1 (50 mg, 0.13 mmol) and 1-methyl-5-(4,4,5,5-tetramethyl-1,3,2-dioxaborolan-2-yl) pyrazole (32 mg, 0.15 mmol) in 1,4-dioxane (2 mL) and water (0.2 mL) was treated with KOAc (51 mg, 0.52 mmol) and the mixture sparged with nitrogen for 10 mins. The reaction mixture was then charged with Pd-118 (8 mg, 0.01 mmol), sealed and heated at 80° C. overnight. The reaction mixture was reduced via Genevac then DCM and water added. The mixture was vigorously agitated before being passed through a phase separator. Solvent was evaporated and the residue purified by flash silica column chromatography on an ISCO system (0-90% EtOAc/PE gradient) to give N-[2-methoxy-6-(2-methylpyrazol-3-yl)-3-pyridyl]-5-methyl-3-phenyl-isoxazole-4-carboxamide (38 mg, 72% yield). ¹H NMR (600 MHZ, Chloroform-d) δ8.71 (d, J=8.1 Hz, 1H), 7.75 (s, 1H), 7.66-7.61 (m, 3H), 7.60-7.57 (m, 2H), 7.45 (d, J=1.9 Hz, 1H), 7.18 (d, J=8.1 Hz, 1H), 6.52 (d, J=1.9 Hz, 1H), 4.17 (s, 3H), 3.65 (s, 3H), 2.83 (s, 3H). m/z 390.0 [M+H]⁺.

### Example 24 N-[2-Methoxy-6-(3-methyl-4-pyridyl)-3-pyridyl]-5-methyl-3-phenyl-isoxazole-4-carboxamide

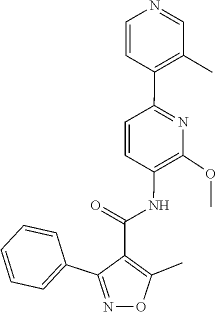 [SMILES: COc1nc(-c2ccncc2C)ccc1NC(=O)c1c(-c2ccccc2)noc1C]

A solution of Intermediate 1 (50 mg, 0.13 mmol) and (3-methyl-4-pyridyl) boronic acid (21 mg, 0.15 mmol) in 1,4-dioxane (2 mL) was treated with KOAc (51 mg, 0.52 mmol) and the mixture sparged with nitrogen for 10 mins. The reaction mixture was then charged with Pd-118 (8 mg, 0.01 mmol), sealed and heated at 80° C. overnight. The reaction mixture was reduced via Genevac then DCM and water added. The mixture was vigorously agitated before being passed through a phase separator. Solvent was evaporated and the residue purified by flash silica column chromatography on an ISCO system with further purification by mass directed preparative HPLC to give N-[2-methoxy-6-(3-methyl-4-pyridyl)-3-pyridyl]-5-methyl-3-phenyl-isoxazole-4-carboxamide (5 mg, 9% yield). ¹H NMR (600 MHZ, Chloroform-d) δ8.75 (d, J=8.0 Hz, 1H), 8.57-8.41 (m, 2H), 7.79 (s, 1H), 7.67-7.61 (m, 3H), 7.60-7.55 (m, 2H), 7.33 (d, J=5.0 Hz, 1H), 7.09 (d, J=8.1 Hz, 1H), 3.64 (s, 3H), 2.84 (s, 3H), 2.42 (s, 3H). m/z 401.0 [M+H]⁺.

### Example 25 N-[6-(3,5-Dimethyl-1H-pyrazol-4-yl)-2-methoxy-3-pyridyl]-5-methyl-3-phenyl-isoxazole-4-carboxamide

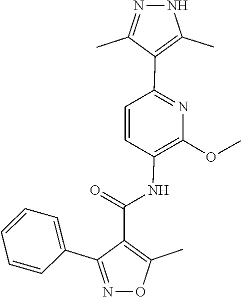 [SMILES: COc1nc(-c2c(C)n[nH]c2C)ccc1NC(=O)c1c(-c2ccccc2)noc1C]

A 5 mL microwave vial was charged with Intermediate 1 (50 mg, 0.13 mmol), 1,3-dimethyl-4-(4,4,5,5-tetramethyl-1,3,2-dioxaborolan-2-yl) pyrazole (34 mg, 0.15 mmol), KOAc (51 mg, 0.52 mmol), 1,4-dioxane (2 mL) and water (0.2 mL). The reaction mixture was degassed for 10 min before adding Pd-118 (8.4 mg, 0.01 mmol) and the vial was sealed and heated at 80° C. for 16 h. The reaction mixture was concentrated under reduced pressure and redissolved in DCM and passed through a hydrophobic frit. The filtrate was concentrated under reduced pressure. The residue was adsorbed onto silica and purified by flash column chromatography (4 g silica, elution with a 0-10% MeOH/EtOAc gradient) to afford N-[6-(3,5-dimethyl-1H-pyrazol-4-yl)-2-methoxy-3-pyridyl]-5-methyl-3-phenyl-isoxazole-4-carboxamide (18 mg, 33% yield) as a colourless solid. ¹H NMR (600 MHZ, Chloroform-d) δ8.64 (d, J=8.1 Hz, 1H), 7.70 (s, 1H), 7.66-7.63 (m, 2H), 7.63-7.59 (m, 1H), 7.57 (dd, J=8.1, 6.6 Hz, 2H), 6.91 (d, J=8.1 Hz, 1H), 3.65 (d, J=1.3 Hz, 3H), 2.83 (s, 3H), 2.44 (d, J=1.2 Hz, 6H). m/z 404.0 [M+H]⁺.

### Example 26 N-[2-Methoxy-6-(1H-pyrazol-4-yl)-3-pyridyl]-5-methyl-3-phenyl-isoxazole-4-carboxamide

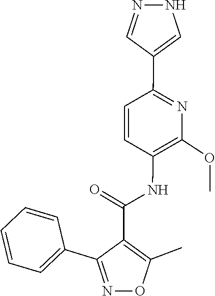 [SMILES: COc1nc(-c2cn[nH]c2)ccc1NC(=O)c1c(-c2ccccc2)noc1C]

A solution of Intermediate 1 (50 mg, 0.13 mmol) and 4-(4,4,5,5-tetramethyl-1,3,2-dioxaborolan-2-yl)-1H-pyrazole (30 mg, 0.15 mmol) in 1,4-dioxane (2 mL) and water (0.2 mL) was treated with KOAc (51 mg, 0.52 mmol) and the mixture sparged with nitrogen for 10 mins. The reaction mixture was then charged with Pd-118 (8 mg, 0.01 mmol), sealed and heated at 80° C. overnight. The reaction mixture was reduced via Genevac then DCM and water added. The mixture was vigorously agitated before being passed through a phase separator. Solvent was evaporated and the residue purified by flash silica column chromatography on an ISCO system (0-10% MeOH/DCM gradient) with trituration of the resultant solid with ether to give N-[2-methoxy-6-(1H-pyrazol-4-yl)-3-pyridyl]-5-methyl-3-phenyl-isoxazole-4-carboxamide (35 mg, 69% yield). ¹H NMR (600 MHZ, Chloroform-d) δ8.63 (d, J=8.1 Hz, 1H), 7.99 (s, 2H), 7.70 (s, 1H), 7.66-7.59 (m, 3H), 7.58-7.55 (m, 2H), 7.06 (d, J=8.1 Hz, 1H), 3.66 (s, 3H), 2.83 (s, 3H). m/z 374.0 [M−H].

### Example 27 N-[6-[6-(Hydroxymethyl)-3-pyridyl]-2-methoxy-3-pyridyl]-5-methyl-3-phenyl-isoxazole-4-carboxamide

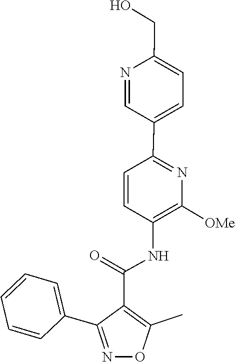 [SMILES: COc1nc(-c2ccc(CO)nc2)ccc1NC(=O)c1c(-c2ccccc2)noc1C]

### Step 1—Synthesis of Intermediate 7-N-[2-Methoxy-6-(4,4,5,5-tetramethyl-1,3,2-dioxaborolan-2-yl)-3-pyridyl]-5-methyl-3-phenyl-isoxazole-4-carboxamide

To a microwave vial under an inert atmosphere was added Intermediate 1 (1.50 g, 3.86 mmol), 4,4,5,5-tetramethyl-2-(4,4,5,5-tetramethyl-1,3,2-dioxaborolan-2-yl)-1,3,2-dioxaborolane (1.28 g, 5.02 mmol) and anhydrous 1,4-dioxane (44 mL). The solution was degassed with nitrogen for 15 min before Pd(dppf)Cl₂ (complex with DCM, 315 mg, 0.380 mmol) and KOAc (1.14 g, 11.59 mmol) were quickly added. The mixture was again degassed with nitrogen for 15 min before being heated to 80° C. for 1 h using an aluminium heating block. The reaction mixture was allowed to cool to room temperature and was diluted with EtOAc (30 mL). The solution was washed with water (20 mL), dried over MgSO₄, filtered and concentrated under reduced pressure to give the crude product which was purified by trituration with PE (60 mL). The suspension was left to settle, the supernatant was removed and the trituration procedure repeated with additional PE (20 mL). The resulting solid was dried under reduced pressure to give N-[2-methoxy-6-(4,4,5,5-tetramethyl-1,3,2-dioxaborolan-2-yl)-3-pyridyl]-5-methyl-3-phenyl-isoxazole-4-carboxamide (Intermediate 7, 1.69 g, 95%) as a brown powder. ¹H NMR (500 MHZ, Chloroform-d) δ8.60 (d, J=7.8 Hz, 1H), 7.82 (s, 1H), 7.64-7.57 (m, 3H), 7.57-7.50 (m, 2H), 7.45 (dd, J=7.8, 1.3 Hz, 1H), 3.66 (s, 3H), 2.81 (s, 3H), 1.34 (s, 12H). m/z 354.0 [M+H for boronic acid]⁺.

### Step 2

A solution of KF (76.1 mg, 1.31 mmol), (5-bromopyrid-2-yl) methanol (82.1 mg, 0.440 mmol) and Intermediate 7 (200 mg, 0.459 mmol) in 1,4-dioxane (4 mL) and water (1 mL) was degassed with nitrogen for 15 min. Pd-118 (28.5 mg, 0.044 mmol) was quickly added, the solution degassed again for 10 min and the mixture left to stir at room temperature overnight. LCMS analysis the morning after indicated the reaction was near-complete, therefore the reaction mixture was heated to 45° C. for 4 h to promote reaction completion. The reaction mixture was diluted with EtOAc (20 mL), washed with water (20 mL). The aqueous phase was back-extracted with DCM (3×20 mL) and the combined organic extract was dried over MgSO₄, filtered and concentrated under reduced pressure to give the crude product, which was purified by automated column chromatography (12 g silica, gradient 0-10% MeOH/DCM) to give N-[6-[6-(hydroxymethyl)-3-pyridyl]-2-methoxy-3-pyridyl]-5-methyl-3-phenyl-isoxazole-4-carboxamide as a grey solid (102 mg, 56% yield); ¹H NMR (500 MHZ, Chloroform-d) δ9.13 (s, 1H), 8.73 (d, J=8.1 Hz, 1H), 8.21 (dd, J=8.2, 2.2 Hz, 1H), 7.77 (s, 1H), 7.66-7.61 (m, 3H), 7.61-7.55 (m, 2H), 7.35 (d, J=8.1 Hz, 1H), 7.29 (d, J=8.2 Hz, 1H), 4.79 (s, 2H), 3.71 (s, 3H), 2.84 (s, 3H). m/z 417.1 [M+H]⁺.

### Example 28 N-[2-Methoxy-6-(1-methyl-6-oxo-3-pyridyl)-3-pyridyl]-5-methyl-3-phenyl-isoxazole-4-carboxamide

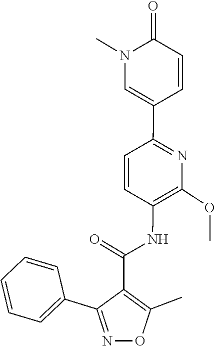 [SMILES: COc1nc(-c2ccc(=O)n(C)c2)ccc1NC(=O)c1c(-c2ccccc2)noc1C]

A solution of Intermediate 1 (50 mg, 0.13 mmol) and 1-methyl-5-(4,4,5,5-tetramethyl-1,3,2-dioxaborolan-2-yl)pyridin-2-one (36 mg, 0.15 mmol) in 1,4-dioxane (2 mL) and water (0.2 mL) was treated with KOAc (51 mg, 0.52 mmol) and the mixture sparged with nitrogen for 10 mins. The reaction mixture was then charged with Pd-118 (8 mg, 0.01 mmol), sealed and heated at 80° C. overnight. The reaction mixture was reduced via Genevac then DCM and water added. The mixture was vigorously agitated before being passed through a phase separator. Solvent was evaporated and the residue purified by flash silica column chromatography on an ISCO system (0-10% MeOH/DCM gradient) with trituration of the resultant solid with ether to give N-[2-methoxy-6-(1-methyl-6-oxo-3-pyridyl)-3-pyridyl]-5-methyl-3-phenyl-isoxazole-4-carboxamide (38 mg, 67%). ¹H NMR (600 MHZ, Chloroform-d) δ8.67 (d, J=8.1 Hz, 1H), 7.98 (d, J=2.5 Hz, 1H), 7.90 (dd, J=9.5, 2.6 Hz, 1H), 7.72 (s, 1H), 7.67-7.60 (m, 3H), 7.59-7.56 m, 2H), 7.06 (d, J=8.2 Hz, 1H), 6.63 (d, J=9.5 Hz, 1H), 3.67 (s, 3H), 3.63 (s, 3H), 2.83 (s, 3H). m/z 417.0 [M+H]⁺

### Example 29 N-(5-Imidazo[1,2-a]pyridin-5-yl-3-methoxy-2-pyridyl)-5-methyl-3-phenyl-isoxazole-4-carboxamide

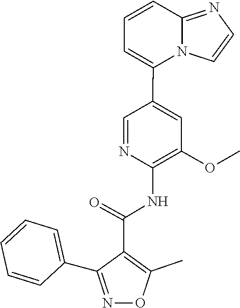 [SMILES: COc1cc(-c2cccc3nccn23)cnc1NC(=O)c1c(-c2ccccc2)noc1C]

To solution of Intermediate 4 (100 mg, 0.24 mmol) in 1,4-dioxane (3 mL) were added B₂Pin₂ (80.8 mg, 0.32 mmol) and KOAc (72 mg, 0.73 mmol). The reaction mixture was degassed by bubbling nitrogen gas for 3 min before adding Pd(dppf)Cl₂ (complex with DCM, 10 mg, 0.01 mmol). The reaction mixture was heated at 90° C. for an hour. The reaction mixture was concentrated and the residue was dissolved in EtOAc (10 mL) and washed with water (3 mL). The organic layer was removed, dried over MgSO₄ and filtered before concentrating under reduced pressure. The resultant crude material (65 mg, 0.15 mmol) was dissolved in 1,4-dioxane (2 mL) and water (0.2 mL). KOAc (44 mg, 0.45 mmol) and 5-bromoimidazo[1,2-a]pyridine (35 mg, 0.18 mmol) were added and the solution was degassed with nitrogen for 15 min. Pd(dppf)Cl₂ (6.1 mg, 0.01 mmol) was quickly added, the solution degassed again for 10 min and the mixture left to stir at 80° C. overnight. Water (10 mL) was added and the reaction mixture extracted with DCM (3×10 mL). The combined organics were washed with brine, dried over MgSO₄ and filtered before concentrating to dryness. Purification by flash silica column chromatography on an ISCO system (elution with 3% MeOH/DCM gradient) to give N-(5-imidazo[1,2-a]pyridin-5-yl-3-methoxy-2-pyridyl)-5-methyl-3-phenyl-isoxazole-4-carboxamide (17 mg, 25% yield) as a white solid. ¹H NMR (600 MHZ, Chloroform-d) δ8.29 (s, 1H), 8.06 (s, 1H), 7.70-7.62 (m, 4H), 7.62-7.52 (m, 4H), 7.25-7.23 (m, 1H), 7.21-7.19 (m, 1H), 6.72 (d, J=6.9 Hz, 1H), 3.56 (s, 3H), 2.86 (s, 3H). m/z 426.11 [M+H]⁺.

### Example 30 N-(6-Imidazo[1,2-a]pyridin-5-yl-2-methoxy-3-pyridyl)-5-methyl-3-phenyl-isoxazole-4-carboxamide

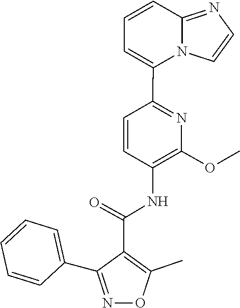 [SMILES: COc1nc(-c2cccc3nccn23)ccc1NC(=O)c1c(-c2ccccc2)noc1C]

A solution of KOAc (31.5 mg, 0.32 mmol), 5-bromoimidazo[1,2-a]pyridine (54 mg, 0.28 mmol) and Intermediate 7 (105 mg, 0.24 mmol) in 1,4-dioxane (2 mL) and water (0.2 mL) was degassed with nitrogen for 15 min. Pd-118 (8.4 mg, 0.01 mmol) was quickly added, the solution degassed again for 10 min and the mixture left to stir at 90° C. for 40 min. Then, water (10 mL) was added and the reaction mixture extracted with DCM (3×10 mL). The combined organics were washed with brine, dried over MgSO₄ and filtered before concentration to dryness. Purification by flash silica column chromatography on an ISCO system (elution with 3% MeOH/DCM gradient) to give N-(6-imidazo[1,2-a]pyridin-5-yl-2-methoxy-3-pyridyl)-5-methyl-3-phenyl-isoxazole-4-carboxamide (28 mg, 24% yield) as a light yellow solid. ¹H NMR (600 MHZ, Chloroform-d) δ8.83 (d, J=8.1 Hz, 1H), 8.39 (s, 1H), 7.85 (s, 1H), 7.66 (d, J=6.6 Hz, 5H), 7.61 (d, J=7.3 Hz, 2H), 7.36 (d, J=8.1 Hz, 1H), 7.23 (d, J=8.3 Hz, 1H), 7.04 (d, J=7.0 Hz, 1H), 3.69 (s, 3H), 2.86 (s, 3H). m/z 426.0 [M+H]⁺.

### Example 31 N-[5-(3,5-Dimethyl-1H-pyrazol-4-yl)-3-methoxy-2-pyridyl]-5-methyl-3-phenyl-isoxazole-4-carboxamide

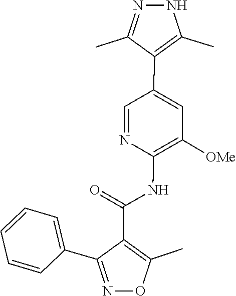 [SMILES: COc1cc(-c2c(C)n[nH]c2C)cnc1NC(=O)c1c(-c2ccccc2)noc1C]

To a microwave vial was added Intermediate 4 (50 mg, 0.13 mmol), 1,4-dioxane (2.0 mL), water (0.2 mL), 3,5-dimethyl-4-(4,4,5,5-tetramethyl-1,3,2-dioxaborolan-2-yl)-1H-pyrazole (34.3 mg, 0.15 mmol) and KOAc (25.3 mg, 0.26 mmol). The reaction mixture was degassed with nitrogen for 3 min before adding Pd-118 (4.2 mg, 0.010 mmol). The vial was sealed and heated at 80° C. overnight. Once cooled, the mixture was concentrated under reduced pressure and the crude product directly purified by automated column chromatography (12 g silica, elution with 2-3% MeOH/DCM gradient) to give N-[5-(3,5-dimethyl-1H-pyrazol-4-yl)-3-methoxy-2-pyridyl]-5-methyl-3-phenyl-isoxazole-4-carboxamide (12.0 mg, 20% yield) as a light yellow solid. ¹H NMR (600 MHZ, Chloroform-d) δ7.95 (s, 1H), 7.91 (s, 1H), 7.67 (d, J=7.2 Hz, 2H), 7.58-7.54 (m, 3H), 6.89 (s, 1H), 3.55 (s, 3H), 2.83 (s, 3H), 2.26 (s, 6H). m/z 404.1 [M+H]⁺.

### Example 32 N-[2-Methoxy-6-(4-methoxy-1H-indazol-6-yl)-3-pyridyl]-5-methyl-3-phenyl-isoxazole-4-carboxamide

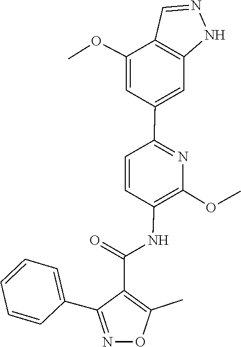

A 5 mL microwave vial was charged with Intermediate 7 (70 mg, 0.16 mmol), 6-bromo-4-methoxy-1H-indazole (36 mg, 0.16 mmol), KOAc (32 mg, 0.32 mmol), 1,4-dioxane (2 mL) and water (0.2 mL). The reaction mixture was degassed with nitrogen before adding Pd-118 (13 mg, 0.02 mmol) and the vial was sealed and heated at 80° C. for 16 h. The reaction mixture was concentrated under reduced pressure and purified by flash column chromatography to afford N-[2-methoxy-6-(4-methoxy-1H-indazol-6-yl)-3-pyridyl]-5-methyl-3-phenyl-isoxazole-4-carboxamide (18 mg, 23% yield) as a yellow solid. ¹H NMR (600 MHZ, DMSO-d₆) δ13.14 (s, 1H), 9.45 (s, 1H), 8.36 (d, J=7.9 Hz, 1H), 8.03 (s, 1H), 7.79 (s, 1H), 7.70 (dd, J=12.2, 7.5 Hz, 3H), 7.55 (d, J=7.6 Hz, 3H), 7.22 (s, 1H), 4.00 (s, 3H), 3.93 (s, 3H), 2.66 (s, 3H). m/z 456.0 [M+H]⁺.

### Example 33 N-[6-(3-Amino-1H-indazol-5-yl)-2-methoxy-3-pyridyl]-5-methyl-3-phenyl-isoxazole-4-carboxamide

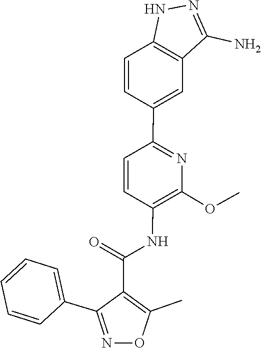 [SMILES: COc1nc(-c2ccc3[nH]nc(N)c3c2)ccc1NC(=O)c1c(-c2ccccc2)noc1C]

A solution of KOAc (31.5 mg, 0.32 mmol), 5-bromo-1H-indazol-3-amine (34 mg, 0.16 mmol) and Intermediate 7 (70 mg, 0.16 mmol) in 1,4-dioxane (2 mL) and water (0.2 mL) was degassed with nitrogen for 15 min. Pd-118 (11.2 mg, 0.01 mmol) was quickly added, the solution degassed again for 10 min and the mixture left to stir at 80° C. for 3 h.

Then, water (10 mL) was added and the reaction mixture extracted with DCM (3×10 mL). The combined organics were washed with brine, dried over MgSO₄ and filtered before concentration to dryness. Purification by flash silica column chromatography to give N-[6-(3-amino-1H-indazol-5-yl)-2-methoxy-3-pyridyl]-5-methyl-3-phenyl-isoxazole-4-carboxamide (10.8 mg, 14% yield) as a yellow solid. ¹H NMR (600 MHZ, DMSO-d₆) δ11.47 (s, 1H), 9.44 (s, 1H), 8.55-8.13 (m, 1H), 7.90 (s, 1H), 7.72 (t, J=8.4 Hz, 3H), 7.61 (d, J=12.4 Hz, 2H), 7.54 (d, J=7.9 Hz, 3H), 5.36 (s, 2H), 3.92 (s, 3H), 2.65 (s, 3H). m/z 441.1 [M+H]⁺.

### Example 34 N-[6-(1H-Indazol-6-yl)-2-methoxy-3-pyridyl]-5-methyl-3-phenyl-isoxazole-4-carboxamide

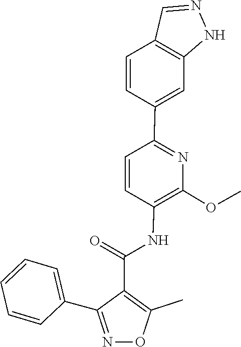

A 5 mL microwave vial was charged with Intermediate 7 (70 mg, 0.16 mmol), 6-bromo-1H-indazole (32 mg, 0.16 mmol), KOAc (32 mg, 0.32 mmol), 1,4-dioxane (2 mL) and water (0.2 mL). The reaction mixture was degassed with nitrogen before adding Pd-118 (13 mg, 0.02 mmol) and the vial was sealed and heated at 80° C. for 16 h. The reaction mixture was concentrated under reduced pressure and purified by flash column chromatography to afford N-[6-(1H-indazol-6-yl)-2-methoxy-3-pyridyl]-5-methyl-3-phenyl-isoxazole-4-carboxamide (13.3 mg, 18% yield) as a yellow solid. ¹H NMR (600 MHZ, DMSO-d₆) δ13.15 (s, 1H), 9.46 (s, 1H), 8.37 (s, 1H), 8.20 (s, 1H), 8.08 (s, 1H), 7.82 (s, 2H), 7.71 (d, J=7.1 Hz, 2H), 7.67 (d, J=8.2 Hz, 1H), 7.59-7.52 (m, 3H), 3.93 (s, 3H), 2.66 (s, 3H). m/z 426.0 [M+H]⁺.

### Example 35 N-[6-(3-Amino-1H-indazol-6-yl)-2-methoxy-3-pyridyl]-5-methyl-3-phenyl-isoxazole-4-carboxamide

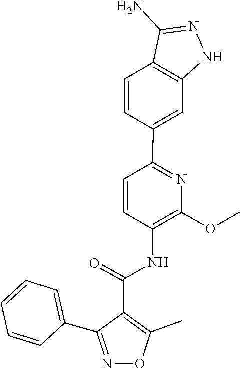 [SMILES: COc1nc(-c2ccc3c(N)n[nH]c3c2)ccc1NC(=O)c1c(-c2ccccc2)noc1C]

A solution of KOAc (31.5 mg, 0.32 mmol), 6-bromo-1H-indazol-3-amine (34 mg, 0.16 mmol) and Intermediate 7 (70 mg, 0.16 mmol) in 1,4-dioxane (2 mL) and water (0.2 mL) was degassed with nitrogen for 15 min. Pd-118 (11.2 mg, 0.01 mmol) was quickly added, the solution degassed again for 10 min and the mixture left to stir at 80° C. for 3 h. Then, water (10 mL) was added and the reaction mixture extracted with DCM (3×10 mL). The combined organics were washed with brine, dried over MgSO₄ and filtered before concentration to dryness. Purification by flash silica column chromatography to give N-[6-(3-amino-1H-indazol-6-yl)-2-methoxy-3-pyridyl]-5-methyl-3-phenyl-isoxazole-4-carboxamide (10.8 mg, 14% yield) as a light yellow solid. ¹H NMR (600 MHZ, DMSO-d₆) δ11.47 (s, 1H), 9.44 (s, 1H), 8.55-8.13 (m, 1H), 7.90 (s, 1H), 7.72 (t, J=8.4 Hz, 3H), 7.61 (d, J=12.4 Hz, 2H), 7.57-7.51 (m, Hz, 3H), 5.36 (s, 2H), 3.92 (s, 3H), 2.65 (s, 3H). m/z 441.1 [M+H]⁺.

### Example 36 N-[6-(2-Chloropyrimidin-5-yl)-2-methoxy-3-pyridyl]-5-methyl-3-phenyl-isoxazole-4-carboxamide

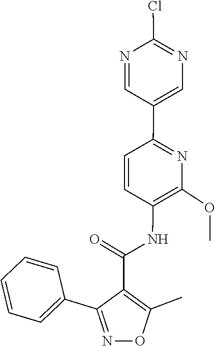 [SMILES: COc1nc(-c2cnc(Cl)nc2)ccc1NC(=O)c1c(-c2ccccc2)noc1C]

A solution of Intermediate 1 (2.00 g, 5.15 mmol) in 1,4-dioxane (5 mL) was sparged with nitrogen for 10 mins. The reaction was then charged with Pd-118 (335 mg, 0.52 mmol) and KF (1.50 g, 25.76 mmol) in one portion. Reaction sealed and sparged with nitrogen for further 10 mins before being heated at 80° C. A degassed suspension of (2-chloropyrimidin-5-yl) boronic acid (1.63 g, 10.30 mmol) in 1,4-dioxane (15 mL) was added dropwise over 12 hours via the use of a syringe pump to the hot solution. Once addition was completed the reaction was kept at 80° C. overnight. Reaction mixture was concentrated under reduced pressure and taken up in DCM (30 mL). Water (30 mL) was added and organics separated using a phase separator. The organics were concentrated under reduced pressure and the residue purified by flash silica column chromatography on an ISCO system (24 g silica, elution with a 0-50% PE/EtOAc gradient, then flushing with 0-10% MeOH/DCM gradient) to give N-[6-(2-chloropyrimidin-5-yl)-2-methoxy-3-pyridyl]-5-methyl-3-phenyl-isoxazole-4-carboxamide (159 mg, 7% yield) as a yellow solid. ¹H NMR (600 MHZ, Chloroform-d) δ9.12 (d, J=1.1 Hz, 2H), 8.79 (d, J=8.1 Hz, 1H), 7.80 (s, 1H), 7.67-7.61 (m, 3H), 7.61-7.56 (m, 2H), 7.35 (d, J=8.2 Hz, 1H), 3.69 (s, 3H), 2.84 (s, 3H). m/z 421.0 [M+H]⁺.

### Example 37 N-(6-Methoxy-[2,3′-bipyridin]-5-yl)-5-methyl-3-phenylisoxazole-4-carboxamide

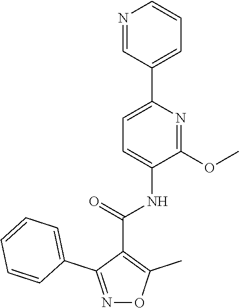 [SMILES: COc1nc(-c2cccnc2)ccc1NC(=O)c1c(-c2ccccc2)noc1C]

A 10 mL microwave vial was charged with N-(6-bromo-2-methoxypyridin-3-yl)-5-methyl-3-phenylisoxazole-4-carboxamide (50.0 mg, 0.13 mmol), 3-(4,4,5,5-tetramethyl-1,3,2-dioxaborolan-2-yl)pyridine (31.7 mg, 0.15 mmol), KOAc (50.6 mg, 0.52 mmol), 1,4-dioxane (2 mL) and water (0.2 mL). The reaction mixture was degassed with nitrogen for 10 mins. Pd-118 (8.4 mg, 0.01 mmol) was quickly added and the mixture further degassed with nitrogen before stirring at 80° C. overnight. The reaction mixture was then concentrated under reduced pressure. The dark residue dissolved in DCM (20 mL) and washed with water (2×10 mL). The organic layer was dried over MgSO₄, filtered and concentrated under reduced pressure. The residue was purified by flash column chromatography (12 g, silica, 0-90% EtOAc/PE over 25 CV's). CV's containing product were combined and evaporated under reduced pressure. The precipitate was then dried under vacuum at 40° C. to afford N-(6-methoxy-[2,3′-bipyridin]-5-yl)-5-methyl-3-phenylisoxazole-4-carboxamide (40.0 mg, 76%) as a pale yellow solid. ¹H NMR (600 MHZ, Chloroform-d) δ9.28-9.14 (m, 1H), 8.74 (d, J=8.1 Hz, 1H), 8.57 (dt, J=4.8, 1.3 Hz, 1H), 8.21 (dq, J=8.1, 1.6 Hz, 1H), 7.78 (s, 1H), 7.63 (ddd, J=8.9, 7.1, 1.3 Hz, 3H), 7.58 (t, J=7.6 Hz, 2H), 7.40-7.31 (m, 2H), 3.71 (s, 3H), 2.84 (s, 3H). m/z 387.0 [M+H]⁺.

### Example 38 N-(6-Imidazo[1,2-a]pyrazin-8-yl-2-methoxy-3-pyridyl)-5-methyl-3-phenyl-isoxazole-4-carboxamide

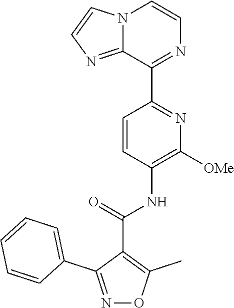 [SMILES: COc1nc(-c2nccn3ccnc23)ccc1NC(=O)c1c(-c2ccccc2)noc1C]

To a 5 mL microwave vial were added 8-chloroimidazo[1,2-a]pyrazine (21.2 mg, 0.14 mmol), Intermediate 7 (60 mg, 0.14 mmol), KOAc (27.0 mg, 0.28 mmol), 1,4-dioxane (2 mL) and water (0.2 mL). The mixture was degassed with nitrogen for 3 min before adding Pd-118 (11.3 mg, 0.02 mmol). The vial was sealed and heated at 80° C. overnight. The mixture was concentrated under reduced pressure and the residue purified by automated column chromatography to give N-(6-imidazo[1,2-a]pyrazin-8-yl-2-methoxy-3-pyridyl)-5-methyl-3-phenyl-isoxazole-4-carboxamide (8.8 mg, 15%). ¹H NMR (600 MHZ, DMSO-d₆) δ9.46 (s, 1H), 8.64 (d, J=4.5 Hz, 1H), 8.50 (s, 1H), 8.40 (d, J=8.0 Hz, 1H), 8.21 (s, 1H), 8.02 (d, J=4.5 Hz, 1H), 7.86 (s, 1H), 7.78-7.65 (m, 2H), 7.63-7.49 (m, 3H), 3.88 (s, 3H), 2.67 (s, 3H). m/z 427.1 [M+H]⁺.

### Example 39 N-[6-(5-Amino-6-methoxy-pyrazin-2-yl)-2-methoxy-3-pyridyl]-5-methyl-3-phenyl-isoxazole-4-carboxamide

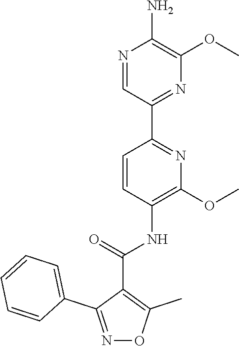 [SMILES: COc1nc(-c2cnc(N)c(OC)n2)ccc1NC(=O)c1c(-c2ccccc2)noc1C]

A solution of KOAc (27 mg, 0.28 mmol), 5-bromo-3-methoxy-pyrazin-2-amine (28 mg, 0.14 mmol) and Intermediate 7 (60 mg, 0.14 mmol) in 1,4-dioxane (2 mL) and water (0.2 mL) was degassed with nitrogen for 15 min. Pd-118 (11.2 mg, 0.01 mmol) was quickly added, the solution degassed again for 10 min and the mixture left to stir at 80° C. for 3 h. Then, water (10 mL) was added and the reaction mixture extracted with DCM (3×10 mL). The combined organics were washed with brine, dried over MgSO₄ and filtered before concentration to dryness. Purification by flash silica column chromatography gave N-[6-(5-amino-6-methoxy-pyrazin-2-yl)-2-methoxy-3-pyridyl]-5-methyl-3-phenyl-isoxazole-4-carboxamide (10 mg, 16% yield) as a white solid. ¹H NMR (600 MHZ, DMSO-d₆) δ9.37 (s, 1H), 8.38 (s, 1H), 8.28 (d, J=7.9 Hz, 1H), 7.70 (d, J=6.9 Hz, 2H), 7.64 (d, J=8.1 Hz, 1H), 7.58-7.50 (m, 3H), 6.62 (s, 2H), 3.99 (s, 3H), 3.86 (s, 3H), 2.65 (s, 3H). m/z 433.1 [M+H]⁺.

### Example 40 N-[6-(6-Aminopyrazin-2-yl)-2-methoxy-3-pyridyl]-5-methyl-3-phenyl-isoxazole-4-carboxamide

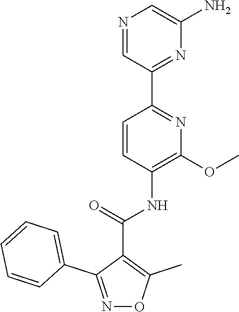 [SMILES: COc1nc(-c2cncc(N)n2)ccc1NC(=O)c1c(-c2ccccc2)noc1C]

A solution of KOAc (27 mg, 0.28 mmol), 5-bromopyrazin-2-amine (24 mg, 0.14 mmol) and Intermediate 7 (60 mg, 0.14 mmol) in 1,4-dioxane (2 mL) and water (0.2 mL) was degassed with nitrogen for 15 min. Pd-118 (11.2 mg, 0.01 mmol) was quickly added, the solution degassed again for 10 min and the mixture left to stir at 80° C. for 3 h. Then, water (10 mL) was added and the reaction mixture extracted with DCM (3×10 mL). The combined organics were washed with brine, dried over MgSO₄ and filtered before concentration to dryness. Purification by flash silica column chromatography gave N-[6-(6-aminopyrazin-2-yl)-2-methoxy-3-pyridyl]-5-methyl-3-phenyl-isoxazole-4-carboxamide (18 mg, 32% yield) as a white solid. ¹H NMR (600 MHZ, DMSO-d₆) δ9.42 (s, 1H), 8.57 (s, 1H), 8.43 (s, 1H), 7.87 (s, 1H), 7.79 (d, J=8.1 Hz, 1H), 7.69 (d, J=7.1 Hz, 2H), 7.62-7.49 (m, 3H), 6.52 (s, 2H), 3.88 (s, 3H), 2.65 (s, 3H). m/z 403.1 [M+H]⁺.

### Example 41 N-(2-Methoxy-6-(2-methoxypyrimidin-5-yl)pyridin-3-yl)-5-methyl-3-phenyl-isoxazole-4-carboxamide

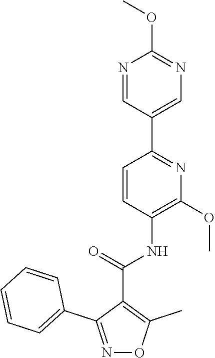 [SMILES: COc1ncc(-c2ccc(NC(=O)c3c(-c4ccccc4)noc3C)c(OC)n2)cn1]

To a solution of Intermediate 1 (190 mg, 0.49 mmol) in dimethoxyethane (3 mL) were added sequentially (2-methoxypyrimidin-5-yl) boronic acid (113 mg, 0.74 mmol), Na₂CO₃ (130 mg, 1.22 mmol) and Pd(dppf)Cl₂ (complexed with DCM, 20 mg, 0.03 mmol). The resulting mixture was stirred at 90° C. overnight. Upon completion of the reaction, volatiles were evaporated under reduced pressure. The residue was dissolved in water (3 mL) and extracted with DCM (3×1 mL). The combined organic layers were separated, dried over Na₂SO₄ and concentrated under reduced pressure to obtain crude product, which was further purified on a C18 HPLC (gradient of 0-100% MeOH/water) to give N-(2-methoxy-6-(2-methoxypyrimidin-5-yl)pyridin-3-yl)-5-methyl-3-phenylisoxazole-4-carboxamide (92 mg, 45% yield). ¹H NMR (600 MHZ, Chloroform-d) δ9.04 (s, 2H), 8.78-8.65 (m, 1H), 7.76 (s, 1H), 7.66-7.61 (m, 3H), 7.61-7.55 (m, 2H), 7.25 (s, 1H), 4.06 (s, 3H), 3.69 (s, 3H), 2.84 (s, 3H). m/z 418.0 [M+H]⁺.

### Example 42 N-(6-Methoxy-[2,4′-bipyridin]-5-yl)-5-methyl-3-phenylisoxazole-4-carboxamide

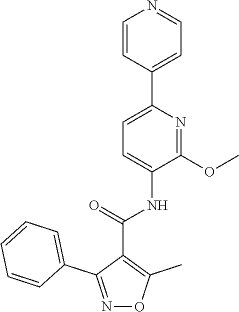 [SMILES: COc1nc(-c2ccncc2)ccc1NC(=O)c1c(-c2ccccc2)noc1C]

A 10 mL microwave vial was charged Intermediate 1 (50.0 mg, 0.13 mmol), 4-(4,4,5,5-tetramethyl-1,3,2-dioxaborolan-2-yl)pyridine (31.7 mg, 0.15 mmol), KOAc (50.6 mg, 0.52 mmol), 1,4-dioxane (2 mL) and water (0.2 mL). The reaction mixture was degassed with nitrogen for 10 mins. Pd-118 (8.4 mg, 0.01 mmol) was quickly added and the mixture further degassed with nitrogen before stirring at 80° C. overnight. The reaction mixture was then concentrated under reduced pressure. The dark residue dissolved in DCM (20 mL) and washed with water (2×10 mL). The organic layer was dried over MgSO₄, filtered and concentrated under reduced pressure. The residue was purified by flash column chromatography (12 g, silica, elution with 0-90% EtOAc/PE) to give N-(6-methoxy-[2,4′-bipyridin]-5-yl)-5-methyl-3-phenylisoxazole-4-carboxamide (26.0 mg, 50%) as a tan solid. ¹H NMR (600 MHZ, Chloroform-d) δ8.76 (d, J=8.1 Hz, 1H), 8.71-8.57 (m, 2H), 7.90-7.74 (m, 3H), 7.67-7.61 (m, 3H), 7.62-7.55 (m, 2H), 7.44 (d, J=8.1 Hz, 1H), 3.71 (s, 3H), 2.84 (s, 3H). m/z 387.0 [M+H]⁺.

### Example 43 N-[6-(2-Aminopyrimidin-5-yl)-2-methoxy-3-pyridyl]-5-methyl-3-phenyl-isoxazole-4-carboxamide

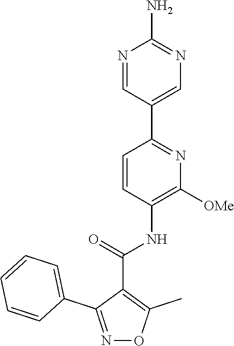 [SMILES: COc1nc(-c2cnc(N)nc2)ccc1NC(=O)c1c(-c2ccccc2)noc1C]

A solution of KF (76.1 mg, 1.31 mmol), 2-amino-5-bromopyrimidine (76.0 mg, 0.44 mmol) and Intermediate 7 (200 mg, 0.44 mmol) in 1,4-dioxane (4 mL) and water (1 mL) was degassed with nitrogen for 15 min. Pd-118 (28.5 mg, 0.04 mmol) was quickly added, the solution degassed again for 10 min and the mixture left to stir at room temperature overnight. LCMS analysis the morning after indicated the reaction was near-complete but both starting materials were still observable-therefore the mixture was heated at 45° C. and monitored by LCMS analysis. LCMS analysis after 4 h showed full consumption of starting materials. The reaction mixture was diluted with EtOAc (20 mL), washed with water (20 mL). The aqueous phase was back-extracted with DCM (3×20 mL). The combined organic extracts were dried over MgSO₄, filtered and concentrated under reduced pressure to give the crude product, which was purified by automated column chromatography (12 g silica, elution with 0-10% MeOH/DCM gradient) to give N-[6-(2-aminopyrimidin-5-yl)-2-methoxy-3-pyridyl]-5-methyl-3-phenyl-isoxazole-4-carboxamide (114 mg, 64% yield) as a beige solid. ¹H NMR (500 MHZ, DMSO-d₆) δ9.45 (s, 1H), 8.90 (s, 2H), 8.27 (d, J=7.8 Hz, 1H), 7.77-7.67 (m, 2H), 7.60-7.52 (m, 3H), 7.49 (d, J=8.1 Hz, 1H), 6.96 (s, 2H), 3.89 (s, 3H), 2.66 (s, 3H). m/z 403.0 [M+H]⁺.

### Example 44 N-(5-Imidazol-1-yl-3-methoxy-pyrazin-2-yl)-5-methyl-3-phenyl-isoxazole-4-carboxamide

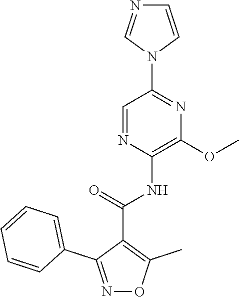 [SMILES: COc1nc(-n2ccnc2)cnc1NC(=O)c1c(-c2ccccc2)noc1C]

To solution of L-proline (4.5 mg, 0.04 mmol) in DMSO (1.5 mL) was added copper(I) iodide (7.4 mg, 0.04 mmol), K₂CO₃ (107 mg, 0.77 mmol) and Intermediate 6 (100 mg, 0.26 mmol). The reaction mixture was degassed with nitrogen before adding imidazole (17 mg, 0.26 mmol). The reaction mixture was heated at 80° C. for 16 h and then diluted with EtOAc (30 mL) and filtered through celite. The filtrate was washed with water (30 mL), brine (30 mL), dried over MgSO₄, filtered and concentrated under reduced pressure. The residue was adsorbed onto silica and purified on a Shimadzu MDAP (elution with a 10-95% MeCN/water+0.1% formic acid gradient) to afford N-(5-imidazol-1-yl-3-methoxy-pyrazin-2-yl)-5-methyl-3-phenyl-isoxazole-4-carboxamide (38 mg, 37% yield) as a cream solid. ¹H NMR (600 MHZ, Chloroform-d) δ8.17 (s, 1H), 8.07 (s, 1H), 7.88 (s, 1H), 7.64-7.59 (m, 3H), 7.57 (t, J=7.5 Hz, 2H), 7.26 (d, J=1.3 Hz, 2H), 3.76 (s, 3H), 2.85 (s, 3H). m/z 377.0 [M+H]⁺.

### Example 45 N-(6-(2-(Dimethylamino)pyrimidin-5-yl)-2-methoxypyridin-3-yl)-5-methyl-3-phenylisoxazole-4-carboxamide

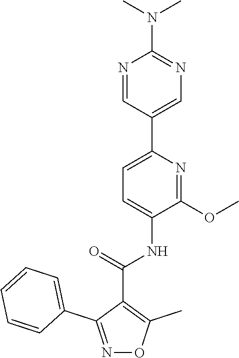 [SMILES: COc1nc(-c2cnc(N(C)C)nc2)ccc1NC(=O)c1c(-c2ccccc2)noc1C]

A 10 mL microwave vial was charged with Example 36 (30.0 mg, 0.07 mmol), dimethylamine (71.1 μL, 0.14 mmol), DIPEA (24.8 μL, 0.14 mmol) and THF (1.5 mL). The reaction mixture was heated to 70° C. and stirred for 2 h. The reaction mixture was concentrated under reduced pressure and the residue purified by flash column chromatography (4 g, silica, elution with 0-100% EtOAc/PE gradient). CV's containing product were combined and evaporated under reduced pressure to give N-(6-(2-(dimethylamino)pyrimidin-5-yl)-2-methoxypyridin-3-yl)-5-methyl-3-phenylisoxazole-4-carboxamide (14.0 mg, 41% yield) as a colourless solid. ¹H NMR (600 MHZ, Chloroform-d) δ8.89 (s, 2H), 8.65 (d, J=8.1 Hz, 1H), 7.70 (s, 1H), 7.66-7.59 (m, 3H), 7.57 (dd, J=7.9, 6.5 Hz, 2H), 7.15 (d, J=8.2 Hz, 1H), 3.66 (s, 3H), 3.24 (s, 6H), 2.83 (s, 3H). m/z 431.0 [M+H]⁺.

### Example 46 N-[2-Methoxy-6-(1,2,4-triazol-1-yl)-3-pyridyl]-5-methyl-3-phenyl-isoxazole-4-carboxamide

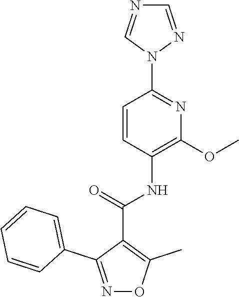 [SMILES: COc1nc(-n2cncn2)ccc1NC(=O)c1c(-c2ccccc2)noc1C]

To a solution of Intermediate 1 (100 mg, 0.25 mmol) in DMSO (2 mL) were added L-proline (3 mg, 0.026 mmol), copper(I) iodide (5 mg, 0.026 mmol), 1,2,4-triazole (21.5 mg, 0.31 mmol) and K₂CO₃ (107 mg, 0.77 mmol). The reaction mixture was evacuated and backfilled with nitrogen three times, then heated at 120° C. overnight. The reaction mixture was diluted with EtOAc (10 mL) and was filtered over celite. The clear filtrate was washed with water (10 mL) and brine (10 mL). The organic layer was then dried over MgSO₄ and filtered before concentration to dryness. Purification by flash silica column chromatography on an ISCO system (elution with 35% EtOAc/PE gradient) gave N-[2-methoxy-6-(1,2,4-triazol-1-yl)-3-pyridyl]-5-methyl-3-phenyl-isoxazole-4-carboxamide (27 mg, 26% yield) as a white solid. ¹H NMR (600 MHZ, Chloroform-d) δ8.93 (s, 1H), 8.86 (d, J=8.4 Hz, 1H), 8.05 (s, 1H), 7.70 (s, 1H), 7.66-7.62 (m, 3H), 7.61-7.56 (m, 2H), 7.44 (d, J=8.4 Hz, 1H), 3.68 (s, 3H), 2.84 (s, 3H). m/z 377.0 [M+H]⁺.

### Example 47 N-[2-Methoxy-6-[2-(methylamino)pyrimidin-5-yl]-3-pyridyl]-5-methyl-3-phenyl-isoxazole-4-carboxamide

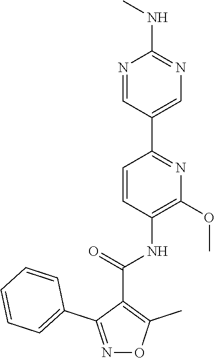 [SMILES: CNc1ncc(-c2ccc(NC(=O)c3c(-c4ccccc4)noc3C)c(OC)n2)cn1]

To a solution of Example 36 (50 mg, 0.12 mmol) in THF (2 mL) was added 2 M methylamine solution in THF (0.18 mL, 0.36 mmol) and DIPEA (0.06 mL, 0.36 mmol). The reaction was stirred at 70° C. overnight. A further portion of methylamine (0.18 mL, 0.36 mmol) and DIPEA (0.06 mL, 0.36 mmol) was added and the reaction continued for a further 7 h. The reaction mixture was concentrated under reduced pressure and resulting solid partitioned between chloroform and water. The solution was passed through a hydrophobic frit and the filtrate evaporated under reduced pressure. The residue was then purified by flash silica column chromatography on an ISCO system (using 0-10% MeOH/DCM gradient) to give N-[2-methoxy-6-[2-(methylamino)pyrimidin-5-yl]-3-pyridyl]-5-methyl-3-phenyl-isoxazole-4-carboxamide (28 mg, 54% yield). ¹H NMR (600 MHZ, Chloroform-d) δ8.86 (s, 2H), 8.68 (d, J=8.4 Hz, 1H), 7.72 (s, 1H), 7.66-7.60 (m, 3H), 7.57 (t, J=7.4 Hz, 2H), 7.16 (d, J=8.2 Hz, 1H), 5.22 (s, 1H), 3.67 (s, 3H), 3.05 (d, J=5.0 Hz, 3H), 2.83 (s, 3H). m/z 417.0 [M+H]⁺.

### Example 48 N-[6-(2-Ethoxypyrimidin-5-yl)-2-methoxy-3-pyridyl]-5-methyl-3-phenyl-isoxazole-4-carboxamide

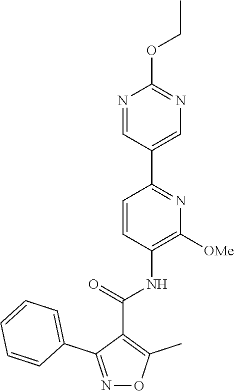 [SMILES: CCOc1ncc(-c2ccc(NC(=O)c3c(-c4ccccc4)noc3C)c(OC)n2)cn1]

A solution of KF (76.1 mg, 1.31 mmol), 5-bromo-2-ethoxy-pyrimidine (93.3 mg, 0.46 mmol) and Intermediate 7 (200 mg, 0.46 mmol) in 1,4-dioxane (4 mL) and water (1 mL) was degassed with nitrogen for 15 min. Pd-118 (28.5 mg, 0.044 mmol) was quickly added, the solution degassed again for 10 min and the mixture left to stir at room temperature overnight. The reaction mixture was diluted with EtOAc (20 mL), washed with water (3×20 mL) and brine (20 mL). The organic extract was dried over MgSO₄, filtered and concentrated under reduced pressure to give the crude product which was purified by automated column chromatography (12 g, silica, gradient from 0-10% MeOH:DCM) to give partially purified product. A trituration in MeOH (5 mL) was performed, the solid left to settle and the supernatant removed by pipette. The solid was further dried in a vacuum oven at 40° C. for 3 h to give N-[6-(2-ethoxypyrimidin-5-yl)-2-methoxy-3-pyridyl]-5-methyl-3-phenyl-isoxazole-4-carboxamide (95 mg, 48%) as a beige solid. ¹H NMR (500 MHZ, Chloroform-d) δ9.04 (s, 2H), 8.74 (d, J=8.2 Hz, 1H), 7.77 (s, 1H), 7.69-7.63 (m, 3H), 7.63-7.57 (m, 2H), 7.30-7.24 (m, 2H), 4.48 (q, J=7.1 Hz, 2H), 3.70 (s, 3H), 2.85 (s, 3H), 1.47 (t, J=7.1 Hz, 3H). m/z 432.1 [M+H]⁺.

### Example 49 N-[6-(1H-Benzotriazol-5-yl)-2-methoxy-3-pyridyl]-5-methyl-3-phenyl-isoxazole-4-carboxamide

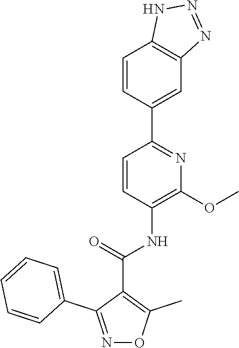 [SMILES: COc1nc(-c2ccc3[nH]nnc3c2)ccc1NC(=O)c1c(-c2ccccc2)noc1C]

A microwave vial was charged with Intermediate 1 (60 mg, 0.15 mmol), 5-(4,4,5,5-tetramethyl-1,3,2-dioxaborolan-2-yl)-1H-benzotriazole (37.8 mg, 0.15 mmol), 1,4-dioxane (2 mL) and water (0.5 mL). The reaction mixture was evacuated and backfilled with nitrogen three times. KOAc (45 mg, 0.45 mmol) and Pd-118 (10 mg, 0.015 mmol) were added. The reaction mixture was then evacuated and backfilled with nitrogen three times before being irradiated under microwave at 120° C. for 30 min. The reaction mixture was concentrated to dryness. The residue was taken up in DCM (10 mL) and the organics were washed with water (10 mL) and brine (10 mL). The collected organics were dried over MgSO₄ and concentrated to dryness under reduced pressure. Purification by flash silica column chromatography on an ISCO system (elution with 3% MeOH/DCM gradient) gave N-[6-(1H-benzotriazol-5-yl)-2-methoxy-3-pyridyl]-5-methyl-3-phenyl-isoxazole-4-carboxamide (10 mg, 14% yield) as a white solid. ¹H NMR (600 MHZ, DMSO-d₆) δ9.47 (s, 1H), 8.55 (s, 1H), 8.39 (s, 1H), 8.18 (d, J=8.6 Hz, 1H), 7.96 (s, 1H), 7.80-7.69 (m, 3H), 7.61-7.50 (m, 2H), 3.94 (s, 3H), 2.66 (s, 3H). m/z 427.0 [M+H]⁺.

### Example 50 N-[6-(2-Amino-[1,2,4]triazolo[1,5-a]pyridin-5-yl)-2-methoxy-3-pyridyl]-5-methyl-3-phenyl-isoxazole-4-carboxamide

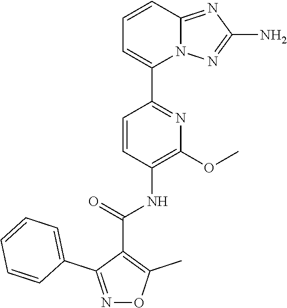 [SMILES: COc1nc(-c2cccc3nc(N)nn23)ccc1NC(=O)c1c(-c2ccccc2)noc1C]

A microwave vial was charged with Intermediate 7 (60 mg, 0.137 mmol), 5-bromo[1,2,4]triazolo[1,5-a]pyridin-2-amine (29.3 mg, 0.140 mmol), 1,4-dioxane (2 mL) and water (0.5 mL). The reaction mixture was evacuated and backfilled with nitrogen three times. Na₂CO₃ (146 mg, 1.37 mmol) and Pd(dppf)Cl₂ (10 mg, 0.014 mmol) were added. The reaction mixture was then evacuated and backfilled with nitrogen three times before being irradiated under microwave at 120° C. for 30 min. The reaction mixture was concentrated to dryness. The residue was taken up in DCM (10 mL) and the organics were washed with water (10 mL) and brine (10 mL). The collected organics were dried over MgSO₄ and concentrated to dryness under reduced pressure. Purification by flash silica column chromatography on an ISCO system (elution with 25% MeOH/DCM gradient containing 1% aq. NH₃) gave N-[6-(2-amino-[1,2,4]triazolo[1,5-a]pyridin-5-yl)-2-methoxy-3-pyridyl]-5-methyl-3-phenyl-isoxazole-4-carboxamide (21 mg, 33% yield) as a white solid. ¹H NMR (600 MHz, DMSO-d₆) δ9.51 (s, 1H), 8.70 (d, J=8.0 Hz, 1H), 8.48 (s, 1H), 7.77-7.65 (m, 3H), 7.60-7.49 m, 4H), 7.41 (d, J=8.6 Hz, 1H), 6.13 (s, 2H), 3.89 (s, 3H), 2.67 (s, 3H). m/z 442.1 [M+H]⁺.

### Example 51 N-[2-Methoxy-6-(1-methyl-1,2,4-triazol-3-yl)-3-pyridyl]-5-methyl-3-phenyl-isoxazole-4-carboxamide

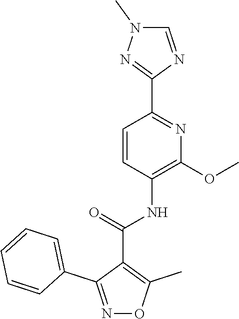 [SMILES: COc1nc(-c2ncn(C)n2)ccc1NC(=O)c1c(-c2ccccc2)noc1C]

A microwave vial was charged with Intermediate 7 (60 mg, 0.14 mmol), 5-bromo-1-methyl-1H-1,2,4-triazole (21.2 mg, 0.13 mmol), 1,4-dioxane (2 mL) and water (1 mL). The reaction mixture was evacuated and backfilled with nitrogen three times. Na₂CO₃ (146 mg, 1.37 mmol) and Pd(dppf)Cl₂ (10 mg, 0.014 mmol) were added. The reaction mixture was then evacuated and backfilled with nitrogen three times before being irradiated under microwave at 120° C. for 30 min. The reaction mixture was concentrated to dryness. The residue was taken up in DCM (10 mL) and the organics were washed with water (10 mL) and brine (10 mL). The collected organics were dried over MgSO₄ and concentrated to dryness under reduced pressure. Purification by flash silica column chromatography on an ISCO system (elution with 3% MeOH/DCM gradient) gave N-[2-methoxy-6-(1-methyl-1,2,4-triazol-3-yl)-3-pyridyl]-5-methyl-3-phenyl-isoxazole-4-carboxamide (17 mg, 32% yield) as a white solid. ¹H NMR (600 MHZ, DMSO-d₆) δ9.41 (s, 1H), 8.51 (s, 1H), 8.38 (s, 1H), 7.75-7.63 (m, 3H), 7.58-7.50 (m, 3H), 3.92 (s, 3H), 3.84 (s, 3H), 2.65 (s, 3H). m/z 391.0 [M+H]⁺.

### Example 52 N-[2-Methoxy-6-(2-methyl-1,2,4-triazol-3-yl)-3-pyridyl]-5-methyl-3-phenyl-isoxazole-4-carboxamide

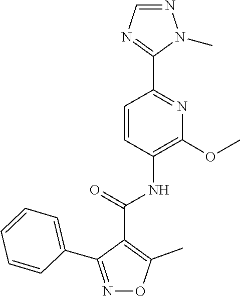 [SMILES: COc1nc(-c2ncnn2C)ccc1NC(=O)c1c(-c2ccccc2)noc1C]

A microwave vial was charged with Intermediate 7 (60 mg, 0.14 mmol), 5-bromo-1-methyl-1H-1,2,4-triazole (21.2 mg, 0.13 mmol), 1,4-dioxane (2 mL) and water (1 mL). The reaction mixture was evacuated and backfilled with nitrogen three times. Na₂CO₃ (146 mg, 1.37 mmol) and Pd(dppf)Cl₂ (10 mg, 0.014 mmol) were added. The reaction mixture was then evacuated and backfilled with nitrogen three times before being irradiated under microwave at 120° C. for 30 min. The reaction mixture was concentrated to dryness. The residue was taken up in DCM (10 mL) and the organics were washed with water (10 mL) and brine (10 mL). The collected organics were dried over MgSO₄ and concentrated to dryness under reduced pressure. Purification by flash silica column chromatography on an ISCO system (elution with 3% MeOH/DCM gradient) gave N-[2-methoxy-6-(2-methyl-1,2,4-triazol-3-yl)-3-pyridyl]-5-methyl-3-phenyl-isoxazole-4-carboxamide (8 mg, 14% yield) as a white solid. ¹H NMR (600 MHZ, Chloroform-d) δ8.82 (d, J=8.2 Hz, 1H), 7.86 (s, 1H), 7.83 (d, J=8.2 Hz, 1H), 7.80 (s, 1H), 7.67-7.63 (m, 3H), 7.62-7.56 m, 2H), 4.30 (s, 3H), 3.67 (s, 3H), 2.84 (s, 3H). m/z 391.2 [M+H]⁺.

### Example 53 N-[2-Methoxy-6-[2-(trifluoromethyl)pyrimidin-5-yl]-3-pyridyl]-5-methyl-3-phenyl-isoxazole-4-carboxamide

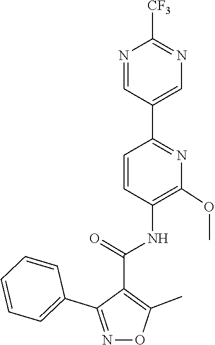 [SMILES: COc1nc(-c2cnc(C(F)(F)F)nc2)ccc1NC(=O)c1c(-c2ccccc2)noc1C]

### Step 1

To solution of 5-bromo-2-(trifluoromethyl)pyrimidine (100 mg, 0.44 mmol) in 1,4-dioxane (5 mL) were added B₂Pin₂ (145 mg, 0.57 mmol). The reaction mixture was degassed with nitrogen for 10 min, followed by addition of Pd(dppf)Cl₂ (complexed with DCM, 18 mg, 0.02 mmol) and KOAc (130 mg, 1.32 mmol). The reaction mixture was degassed for a further 10 min before being heated to 90° C. for 1.5 h. The reaction mixture was concentrated under reduced pressure and the residue was dissolved in EtOAc (10 mL) and washed with water (10 mL). The organic layer was removed, dried over MgSO₄ before evaporation to dryness and trituration of the resultant solid with ether, which was used directly in next step without further purification.

### Step 2

A solution of Intermediate 1 (50 mg, 0.13 mmol) and crude 5-(4,4,5,5-tetramethyl-1,3,2-dioxaborolan-2-yl)-2-(trifluoromethyl)pyrimidine (42 mg, 0.15 mmol) in 1,4-dioxane (2 mL) and water (0.2 mL) was treated with KOAc (51 mg, 0.52 mmol) and the mixture sparged with nitrogen for 10 mins. The reaction was then charged with Pd-118 (8 mg, 0.01 mmol), sealed and heated at 80° C. overnight. The reaction mixture was concentrated under reduced pressure and taken up in DCM (10 mL). Water (10 mL) was added and organics separated using a phase separator. The organics were then concentrated under reduced pressure and the residue purified by flash silica column chromatography on an ISCO system (12 g silica, elution with 0-50% PE/EtOAc gradient) to give N-[2-methoxy-6-[2-(trifluoromethyl)pyrimidin-5-yl]-3-pyridyl]-5-methyl-3-phenyl- isoxazole-4-carboxamide (32 mg, 52% yield). ¹H NMR (600 MHZ, Chloroform-d) δ9.38 (s, 2H), 8.83 (d, J=8.1 Hz, 1H), 7.83 (s, 1H), 7.68-7.62 (m, 3H), 7.62-7.58 (m, 2H), 7.44 (d, J=8.1 Hz, 1H), 3.70 (s, 3H), 2.85 (s, 3H). m/z 456.0 [M+H]⁺.

### Example 54 N-[6-(3-Aminopyrazin-2-yl)-2-methoxy-3-pyridyl]-5-methyl-3-phenyl-isoxazole-4-carboxamide

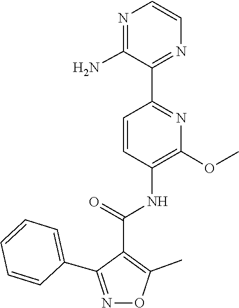 [SMILES: COc1nc(-c2nccnc2N)ccc1NC(=O)c1c(-c2ccccc2)noc1C]

A solution of Na₂CO₃ (36 mg, 0.33 mmol), 3-bromopyrazin-2-amine (22 mg, 0.13 mmol) and Intermediate 7 (50 mg, 0.12 mmol) in 1,4-dioxane (2 mL) and water (1 mL) was degassed with nitrogen for 15 min. Pd-118 (8.4 mg, 0.013 mmol) was quickly added, the solution degassed again for 10 min and the mixture left to stir at 120° C. for 1 h. Water (10 mL) was added and the reaction mixture extracted with DCM (3×10 mL). The combined organics were washed with brine (10 mL), dried over MgSO₄ and filtered before concentration to dryness. Purification by flash silica column chromatography on an ISCO system (elution with 2% MeOH/DCM gradient) gave N-[6-(3-aminopyrazin-2-yl)-2-methoxy-3-pyridyl]-5-methyl-3-phenyl-isoxazole-4-carboxamide (18 mg, 37% yield) as yellow solid. ¹H NMR (600 MHZ, Chloroform-d) δ8.83 (d, J=8.4 Hz, 1H), 8.11 (d, J=8.5 Hz, 1H), 7.97 (d, J=2.4 Hz, 1H), 7.94 (d, J=2.4 Hz, 1H), 7.76 (s, 1H), 7.66-7.62 (m, 3H), 7.61-7.57 (m, 2H), 6.78 (s, 2H), 3.67 (s, 3H), 2.84 (s, 3H). m/z 403.1 [M+H]⁺.

### Example 55 N-[6-(5-Aminopyrazin-2-yl)-2-methoxy-3-pyridyl]-5-methyl-3-phenyl-isoxazole-4-carboxamide hydrochloride

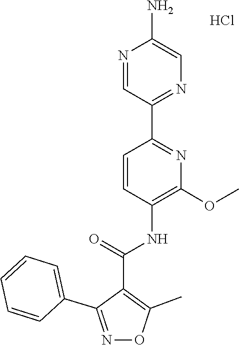 [SMILES: COc1nc(-c2cnc(N)cn2)ccc1NC(=O)c1c(-c2ccccc2)noc1C ; Cl]

A solution of Na₂CO₃ (146 mg, 1.37 mmol), 5-bromopyrazin-2-amine (88 mg, 0.50 mmol) and Intermediate 7 (200 mg, 0.46 mmol) in 1,4-dioxane (8 mL) and water (4 mL) was degassed with nitrogen for 15 min. Pd-118 (33.6 mg, 0.046 mmol) was quickly added, the solution degassed again for 10 min and the mixture left to stir at 100° C. for 1 h. Water (10 mL) was added and the reaction mixture extracted with DCM (3×10 mL). The combined organics were washed with brine (10 mL), dried over MgSO₄ and filtered before concentration to dryness. Purification by flash silica column chromatography on an ISCO system (elution with 2% MeOH/DCM gradient) yielded the free base that was subsequently treated with HCl (1.2 eq.) to give N-[6-(5-aminopyrazin-2-yl)-2-methoxy-3-pyridyl]-5-methyl-3-phenyl-isoxazole-4-carboxamide hydrochloride (23 mg, 11% yield) as a yellow solid. ¹H NMR (600 MHZ, DMSO-d₆) δ9.40 (s, 1H), 8.77 (s, 1H), 8.30 (d, J=8.0 Hz, 1H), 7.91 (d, J=1.4 Hz, 1H), 7.77-7.68 (m, 2H), 7.66 (d, J=8.0 Hz, 1H), 7.60-7.47 (m, 3H), 6.73 (s, 2H), 3.87 (s, 3H), 2.64 (s, 3H). m/z 403.2 [M+H for free base]⁺.

### Example 56 N-(3-Methoxy-5-(pyrimidin-5-yl) pyrazin-2-yl)-5-methyl-3-phenylisoxazole-4-carboxamide

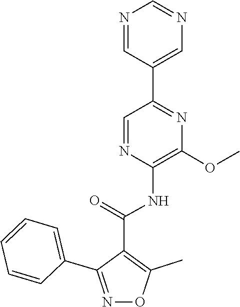 [SMILES: COc1nc(-c2cncnc2)cnc1NC(=O)c1c(-c2ccccc2)noc1C]

A 5 mL microwave vial was charged with Intermediate 6 (30 mg, 0.08 mmol), pyrimidin-5-ylboronic acid (19 mg, 0.15 mmol), potassium phosphate tribasic (55 mg, 0.26 mmol) and Pd(dppf)Cl₂ (complexed with DCM, 10.6 mg, 0.01 mmol). The vial was sealed, purged with nitrogen, and then treated with 1,4-dioxane (2 mL) and water (0.2 mL). The reaction mixture was heated at 120° C. for 16 h. The reaction mixture was concentrated under reduced pressure. The residue was dissolved in EtOAc (10 mL), washed with water (10 mL), brine (10 mL), dried over MgSO₄, filtered and concentrated under reduced pressure. The residue was then purified by reverse phase column chromatography (12 g, C18 cartridge, elution with a 10-100% MeOH/water gradient) and the relevant fractions were concentrated to afford an aqueous solution. The solution was extracted with EtOAc (20 mL) and the organic extract was washed with brine (10 mL), dried over MgSO₄, filtered and organics removed under reduced pressure. The solids were suspended in water (10 mL), filtered via vacuum filtration and washed with water to afford N-(3-methoxy-5-(pyrimidin-5-yl) pyrazin-2-yl)-5-methyl-3-phenylisoxazole-4-carboxamide (8.2 mg, 26% yield) as an off-white solid. ¹H NMR (600 MHZ, Chloroform-d) δ9.23 (s, 3H), 8.42 (s, 1H), 8.02 (s, 1H), 7.66-7.60 (m, 3H), 7.60-7.55 (m, 2H), 3.77 (s, 3H), 2.86 (s, 3H). m/z 389.1 [M+H]⁺.

### Example 57 N-[6-(2-Amino-[1,2,4]triazolo[1,5-a]pyridin-6-yl)-2-methoxy-3-pyridyl]-5-methyl-3-phenyl-isoxazole-4-carboxamide

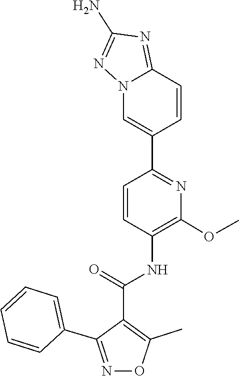 [SMILES: COc1nc(-c2ccc3nc(N)nn3c2)ccc1NC(=O)c1c(-c2ccccc2)noc1C]

A microwave vial was charged with Intermediate 7 (60 mg, 0.138 mmol), 6-bromo[1,2,4]triazolo[1,5-a]pyridin-2-amine (29 mg, 0.137 mmol), 1,4-dioxane (1.2 mL) and water (0.3 mL). The reaction mixture was evacuated and backfilled with nitrogen three times. Na₂CO₃ (146 mg, 1.37 mmol) and Pd(dppf)Cl₂ (10 mg, 0.014 mmol) were added. The reaction mixture was then evacuated and backfilled with nitrogen three times before being irradiated under microwave at 120° C. for 30 min. The reaction mixture was concentrated to dryness. The residue was taken up in DCM (10 mL) and the organics were washed with water (10 mL) and brine (10 mL). The collected organics were dried over MgSO₄ and concentrated to dryness under reduced pressure. Purification by flash silica column chromatography on an ISCO system (elution with 3% MeOH/DCM gradient) yielded N-[6-(2-amino-[1,2,4]triazolo[1,5-a]pyridin-6-yl)-2-methoxy-3-pyridyl]-5-methyl-3-phenyl-isoxazole-4-carboxamide (20 mg, 31% yield) as a white solid. ¹H NMR (600 MHZ, Chloroform-d) δ8.97 (s, 1H), 8.73 (d, J=8.2 Hz, 1H), 7.93 (d, J=9.2 Hz, 1H), 7.76 (s, 1H), 7.68-7.62 (m, 3H), 7.59 (t, J=7.6 Hz, 2H), 7.41 (d, J=9.2 Hz, 1H), 7.27 (s, 1H), 4.50 (s, 2H), 3.70 (s, 3H), 2.84 (s, 3H). m/z 442.2 [M+H]⁺.

### Example 58 N-[2-Methoxy-6-(1,3,4-oxadiazol-2-yl)-3-pyridyl]-5-methyl-3-phenyl-isoxazole-4-carboxamide

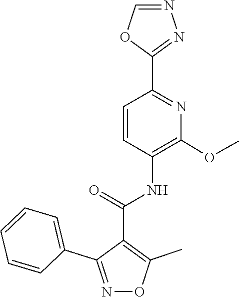 [SMILES: COc1nc(-c2nnco2)ccc1NC(=O)c1c(-c2ccccc2)noc1C]

### Step 1

A 5 mL microwave vial was charged with palladium (II) acetate (3.5 mg, 0.02 mmol) and tri-tert-butylphosphonium tetrafluoroborate (18 mg, 0.06 mmol). The vial was flushed with nitrogen then treated with a solution of phenyl formate (126 mg, 1.03 mmol), Intermediate 1 (200 mg, 0.52 mmol) and Et₃N (0.14 mL, 1.03 mmol) in MeCN (2 mL). The vial was sealed, purged with nitrogen and heated to 80° C. for 16 h. The reaction mixture was partitioned between EtOAc (30 mL) and water (30 mL) and the layers were separated. The organic layer was dried over MgSO₄, filtered and concentrated under reduced pressure. The residue was adsorbed onto silica and purified by flash column chromatography (12 g silica, elution with a 0-90% EtOAc/PE gradient) to afford phenyl 6-methoxy-5-[(5-methyl-3-phenyl-isoxazole-4-carbonyl)amino]pyridine-2-carboxylate (118 mg, 51%) as a colourless solid. ¹H NMR (600 MHZ, Chloroform-d) δ8.82 (d, J=8.1 Hz, 1H), 7.93 (s, 1H), 7.91 (d, J=8.1 Hz, 1H), 7.67-7.61 (m, 3H), 7.61-7.55 (m, 2H), 7.44-7.39 (m, 2H), 7.30-7.24 (m, 1H), 7.21 (d, J=8.1 Hz, 2H), 3.71 (s, 3H), 2.85 (s, 3H). m/z 430.0 [M+H]⁺.

### Step 2

A 5 mL microwave vial was charged with phenyl 6-methoxy-5-[(5-methyl-3-phenyl-isoxazole-4-carbonyl)amino]pyridine-2-carboxylate (80 mg, 0.19 mmol), hydrazine hydrate (0.01 mL, 0.22 mmol) and EtOH (2 mL). The vial was sealed and heated at 100° C. in a microwave for 30 min. The volatiles were removed under reduced pressure. The residue was adsorbed onto silica and purified by flash column chromatography (12 g silica, elution with a 0-90% EtOAc/PE gradient) to afford N-[6-(hydrazinecarbonyl)-2-methoxy-3-pyridyl]-5-methyl-3-phenyl-isoxazole-4-carboxamide (55 mg, 76%) as a colourless solid. ¹H NMR (600 MHZ, Chloroform-d) δ8.80 (d, J=8.1 Hz, 1H), 8.51 (s, 1H), 7.81 (s, 1H), 7.78 (d, J=8.1 Hz, 1H), 7.66-7.60 (m, 3H), 7.58 (t, J=7.5 Hz, 2H), 4.02 (s, 2H), 3.61 (s, 3H), 2.83 (s, 3H). m/z 368.0 [M+H]⁺.

### Step 3

A 5 mL microwave vial was charged with N-[6-(hydrazinecarbonyl)-2-methoxy-3-pyridyl]-5-methyl-3-phenyl-isoxazole-4-carboxamide (51 mg, 0.14 mmol) and trimethyl orthoformate (2.0 mL, 18 mmol) followed by p-toluenesulfonic acid monohydrate (2.6 mg, 0.01 mmol). The vial was sealed, purged with nitrogen and heated at 100° C. in a microwave for 10 min. The reaction mixture was concentrated under reduced pressure. The residue was dissolved in DCM (5 mL) and washed with water (2 mL). The residue was adsorbed onto silica and purified by flash column chromatography (4 g silica, elution with a 0-90% EtOAc/PE gradient) to afford N-[2-methoxy-6-(1,3,4-oxadiazol-2-yl)-3-pyridyl]-5-methyl-3-phenyl-isoxazole-4-carboxamide (42 mg, 77%) as a colourless solid. ¹H NMR (600 MHZ, Chloroform-d) δ8.84 (d, J=8.2 Hz, 1H), 8.46 (s, 1H), 7.88 (s, 1H), 7.80 (d, J=8.2 Hz, 1H), 7.67-7.61 (m, 3H), 7.61-7.57 (m, 2H), 3.71 (s, 3H), 2.84 (s, 3H). m/z 378.0 [M+H]⁺.

### Example 59 N-[2-Methoxy-6-[2-(2,2,2-trifluoroethoxy)pyrimidin-5-yl]-3-pyridyl]-5-methyl-3-phenyl-isoxazole-4-carboxamide

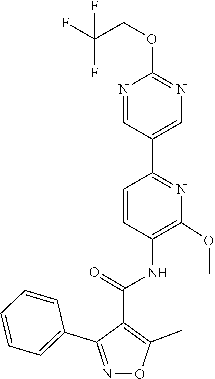 [SMILES: COc1nc(-c2cnc(OCC(F)(F)F)nc2)ccc1NC(=O)c1c(-c2ccccc2)noc1C]

To Example 36 (30 mg, 0.07 mmol) in 2,2,2-trifluoroethanol (2 mL, 27.45 mmol) was added KOH (40 mg, 0.71 mmol) and the reaction was stirred at 50° C. for 2 h. The reaction mixture was poured into water and the solid isolated by filtration, washed with cold ether and dried in a vacuum oven to give N-[2-methoxy-6-[2-(2,2,2-trifluoroethoxy)pyrimidin-5-yl]-3-pyridyl]-5-methyl-3-phenyl-isoxazole-4-carboxamide (16 mg, 44% yield). ¹H NMR (600 MHZ, Chloroform-d) δ9.06 (s, 2H), 8.75 (d, J=8.1 Hz, 1H), 7.77 (s, 1H), 7.66-7.61 (m, 3H), 7.59 (t, J=7.6 Hz, 2H), 7.28 (d, J=8.2 Hz, 1H), 4.86 (q, J=8.3 Hz, 2H), 3.69 (s, 3H), 2.84 (s, 3H). m/z 486.0 [M+H]⁺.

### Example 60 5-[6-Methoxy-5-[(5-methyl-3-phenyl-isoxazole-4-carbonyl)amino]-2-pyridyl]pyrimidine-2-carboxylic acid

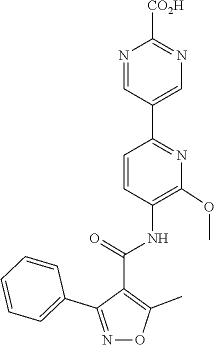 [SMILES: COc1nc(-c2cnc(C(=O)O)nc2)ccc1NC(=O)c1c(-c2ccccc2)noc1C]

In a 25 mL microwave vial Example 70 (187.6 mg, 0.42 mmol) was suspended in THF (9 mL) and water (3 mL). LiOH (176.7 mg, 4.21 mmol) was added and the reaction mixture was stirred at room temperature for 2.5 h. LCMS of reaction mixture showed complete conversion of starting material to product. Organics were removed under reduced pressure and the remaining aqueous solution was acidified to pH 1 with 1M HCl. The solids were filtered under vacuum and washed with water (10 mL), then MeOH (10 mL). The sample was further dried in a vacuum oven at 40° C. overnight to give 5-[6-methoxy-5-[(5-methyl-3-phenyl-isoxazole-4-carbonyl)amino]-2-pyridyl]pyrimidine-2-carboxylic acid (143.7 mg, 75% yield). ¹H NMR (500 MHZ, Chloroform-d) δ9.44 (s, 2H), 8.84 (d, J=8.0 Hz, 1H), 7.85 (s, 1H), 7.71-7.55 (m, 5H), 7.49 (d, J=8.0 Hz, 1H), 3.72 (s, 3H), 2.85 (s, 3H). m/z 432.0 [M+H]⁺.

### Example 61 N-[6-(2-Amino-[1,2,4]triazolo[1,5-a]pyridin-7-yl)-2-methoxy-3-pyridyl]-5-methyl-3-phenyl-isoxazole-4-carboxamide hydrochloride

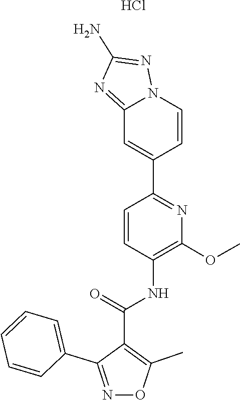 [SMILES: Cl ; COc1nc(-c2ccn3nc(N)nc3c2)ccc1NC(=O)c1c(-c2ccccc2)noc1C]

A microwave vial was charged with Intermediate 7 (60 mg, 0.138 mmol), 7-bromo[1,2,4]triazolo[1,5-a]pyridin-2-amine (29 mg, 0.137 mmol), 1,4-dioxane (1.2 mL) and water (0.3 mL). The reaction mixture was evacuated and backfilled with nitrogen three times. Na₂CO₃ (146 mg, 1.37 mmol) and Pd(dppf)Cl₂ (10 mg, 0.014 mmol) were added. The reaction mixture was then evacuated and backfilled with nitrogen three times before being irradiated under microwave at 120° C. for 1 h. The reaction mixture was concentrated to dryness. The residue was taken up in DCM (10 mL) and the organics were washed with water (10 mL) and brine (10 mL). The collected organics were dried over MgSO₄, filtered and concentrated to dryness under reduced pressure. Purification by flash silica column chromatography on an ISCO system (elution with 40% EtOAc/PE gradient) yielded the free base as an off-white solid. The solid was dissolved in DCM (1 mL) followed by addition of 1M HCl in ether (0.1 mL). Instantaneous formation of solid was noted. The reaction was stirred for 1 h. Then, the reaction mixture was concentrated to dryness under reduced pressure and the solid was further triturated with ether (3 mL). The solid was filtered and washed with ice cooled ether (2 mL) and dried under reduced pressure to give N-[6-(2-amino-[1,2,4]triazolo[1,5-a]pyridin-7-yl)-2-methoxy-3-pyridyl]-5-methyl-3-phenyl-isoxazole-4-carboxamide hydrochloride (27 mg, 33% yield) as a white solid. ¹H NMR (600 MHZ, DMSO-d₆) δ9.52 (s, 1H), 8.79 (d, J=7.0 Hz, 1H), 8.47 (s, 1H), 8.16 (s, 1H), 7.94-7.81 (m, 2H), 7.69 (d, J=7.2 Hz, 2H), 7.60-7.50 (m, 3H), 3.92 (s, 3H), 2.66 (s, 3H). m/z 442.2 [M+H for free base]⁺.

### Example 62 N-[2-Methoxy-6-(1,2,4-triazol-4-yl)-3-pyridyl]-5-methyl-3-phenyl-isoxazole-4-carboxamide

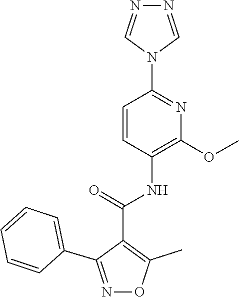 [SMILES: COc1nc(-n2cnnc2)ccc1NC(=O)c1c(-c2ccccc2)noc1C]

### Step 1

A microwave vial was charged with Intermediate 1 (250 mg, 0.64 mmol), L-proline (14.8 mg, 0.13 mmol), ammonium bicarbonate (255 mg, 3.2 mmol) and DMSO (2 mL). The reaction mixture was evacuated and backfilled three times before adding copper(I) iodide (24.5 mg, 0.13 mmol). Then, the reaction mixture was heated at 80° C. overnight. Subsequently, the reaction mixture was diluted with EtOAc (20 mL) followed by washing with water (3×20 mL). The organic layer was then washed with brine, dried over MgSO₄, filtered and concentrated under reduced pressure. Purification by flash silica column chromatography on an ISCO system (elution with 35% EtOAc/PE gradient) yielded N-(6-amino-2-methoxy-3-pyridyl)-5-methyl-3-phenyl-isoxazole-4-carboxamide (100 mg, 45% yield) as a white solid. ¹H NMR (600 MHZ, Chloroform-d) δ8.36 (d, J=8.3 Hz, 1H), 7.66-7.60 (m, 2H), 7.58-7.51 (m, 3H), 7.39 (s, 1H), 6.04 (d, J=8.4 Hz, 1H), 4.13 (br s, 2H), 3.56 (s, 3H), 2.79 (s, 3H). m/z 325.2 [M+H]⁺.

### Step 2

N-(6-Amino-2-methoxy-3-pyridyl)-5-methyl-3-phenyl-isoxazole-4-carboxamide (100 mg, 0.29 mmol) and 1,2-diformylhydrazine (25.7 mg, 0.29 mmol) were mixed in a microwave vial and heated at 150° C. for 1 h. To the reaction mixture, water (10 mL) was added and extraction was carried out with EtOAc (3×10 mL). The collected organics were washed with brine (10 mL), dried over MgSO₄, filtered and evaporated under reduced pressure. Purification by flash silica column chromatography on an ISCO system (elution with 3% MeOH/DCM gradient) yielded N-[2-methoxy-6-(1,2,4-triazol-4-yl)-3-pyridyl]-5-methyl-3-phenyl-isoxazole-4-carboxamide (26 mg, 22% yield) as a light yellow solid. ¹H NMR (600 MHz, Chloroform-d) δ8.86 (d, J=8.3 Hz, 1H), 8.72 (s, 2H), 7.72 (s, 1H), 7.68-7.54 (m, 5H), 6.95 (d, J=8.3 Hz, 1H), 3.67 (s, 3H), 2.84 (s, 3H). m/z 377.0 [M+H]⁺.

### Example 63 N-(2-Methoxy-6-pyrimidin-2-yl-3-pyridyl)-5-methyl-3-phenyl-isoxazole-4-carboxamide

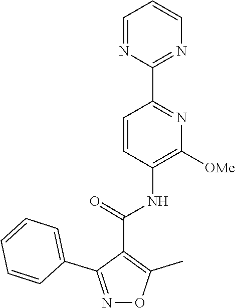 [SMILES: COc1nc(-c2ncccn2)ccc1NC(=O)c1c(-c2ccccc2)noc1C]

A solution of Intermediate 7 (75 mg, 0.170 mmol), 2-bromopyrimidine (27 mg, 0.170 mmol) and KF (123 mg, 2.12 mol) in 1,4-dioxane (4 mL) and water (1 mL) was degassed with nitrogen for 15 min. Pd-118 (45.8 mg, 0.070 mmol) was added, the solution degassed for a further 10 min and then the mixture was left to stir for 4 days at room temperature. The reaction mixture was diluted with EtOAc (20 mL) and washed with water (3×20 mL) and brine (20 mL). The organic extract was dried over MgSO₄, filtered and concentrated under reduced pressure to give the crude product, which was purified by flash silica column chromatography (elution with 50-100% EtOAc/n-hexane gradient) to give N-(2-methoxy-6-pyrimidin-2-yl-3-pyridyl)-5-methyl-3-phenyl-isoxazole-4-carboxamide (41 mg, 58% yield) as a beige solid. ¹H NMR (500 MHZ, Chloroform-d) δ8.85 (d, J=4.8 Hz, 2H), 8.82 (d, J=8.2 Hz, 1H), 8.12 (d, J=8.2 Hz, 1H), 7.89 (s, 1H), 7.66-7.60 (m, 3H), 7.57 (dd, J=8.0, 6.5 Hz, 2H), 7.23 (t, J=4.8 Hz, 1H), 3.78 (s, 3H), 2.84 (s, 3H). m/z 388.1 [M+H]⁺.

### Example 64 N-(2-Methoxy-6-pyridazin-3-yl-3-pyridyl)-5-methyl-3-phenyl-isoxazole-4-carboxamide

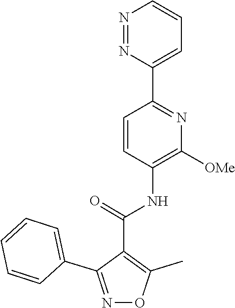 [SMILES: COc1nc(-c2cccnn2)ccc1NC(=O)c1c(-c2ccccc2)noc1C]

A solution of Intermediate 7 (75 mg, 0.170 mmol), 3-bromopyridazine (27.4 mg, 0.170 mmol) and KF (123 mg, 2.12 mmol) in 1,4-dioxane (2 mL) and water (0.2 mL) was degassed with nitrogen for 15 min. Pd-118 (46 mg, 0.07 mmol) was added, the solution degassed for a further 10 min before being left to stir at room temperature for 24 h. The reaction mixture was diluted with EtOAc (20 mL), washed with water (3×20 mL) and brine (20 mL). The organic extract was dried over MgSO₄, filtered and concentrated under reduced pressure to give the crude product, which was purified by flash silica column chromatography (elution with 50-100% EtOAc/n-hexane gradient) to give N-(2-methoxy-6-pyridazin-3-yl-3-pyridyl)-5-methyl-3-phenyl-isoxazole-4-carboxamide (17 mg, 24% yield) as an off-white solid. ¹H NMR (500 MHZ, Chloroform-d) δ9.12 (dd, J=4.9, 1.8 Hz, 1H), 8.84 (d, J=8.3 Hz, 1H), 8.36-8.28 (m, 2H), 7.82 (s, 1H), 7.67-7.61 (m, 3H), 7.61-7.56 (m, 2H), 7.51 (dd, J=8.5, 4.9 Hz, 1H), 3.70 (s, 3H), 2.84 (s, 3H). m/z 388.1 [M+H]⁺.

### Example 65 N-(2-Methoxy-6-pyrimidin-4-yl-3-pyridyl)-5-methyl-3-phenyl-isoxazole-4-carboxamide

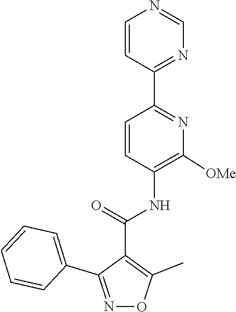 [SMILES: COc1nc(-c2ccncn2)ccc1NC(=O)c1c(-c2ccccc2)noc1C]

A solution of Intermediate 7 (75 mg, 0.170 mmol), 4-chloropyrimidine (20 mg, 0.170 mmol) and KF (123 mg, 2.12 mmol) in 1,4-dioxane (2 mL) and water (0.2 mL) was degassed with nitrogen for 15 min. Pd-118 (45.8 mg, 0.070 mmol) was added, the solution degassed for a further 10 min before being heated to 50° C. overnight. The reaction mixture was diluted with EtOAc (20 mL), washed with water (3×20 mL) and brine (20 mL). The organic extract was dried over MgSO₄, filtered and concentrated under reduced pressure to give the crude product, which was purified by flash silica column chromatography (elution with 50%-100% EtOAc/n-hexane gradient) to give N-(2-methoxy-6-pyrimidin-4-yl-3-pyridyl)-5-methyl-3-phenyl-isoxazole-4-carboxamide (17 mg, 24% yield) as a light brown solid. ¹H NMR (500 MHz, Chloroform-d) δ9.20 (d, J=1.3 Hz, 1H), 8.82 (d, J=8.2 Hz, 1H), 8.77 (d, J=5.3 Hz, 1H), 8.14 (dd, J=5.3, 1.4 Hz, 1H), 8.12 (d, J=8.2 Hz, 1H), 7.86 (s, 1H), 7.67-7.62 (m, 3H), 7.62-7.54 (m, 2H), 3.71 (s, 3H), 2.84 (s, 3H). m/z 388.1 [M+H]⁺.

### Example 66 N-(2-Methoxy-6-thiazol-2-yl-3-pyridyl)-5-methyl-3-phenyl-isoxazole-4-carboxamide

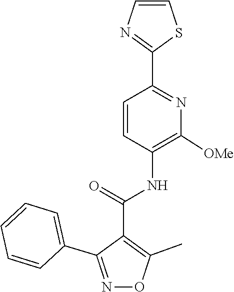 [SMILES: COc1nc(-c2nccs2)ccc1NC(=O)c1c(-c2ccccc2)noc1C]

A solution of 2-bromo-1,3-thiazole (37.7 mg, 0.230 mmol), Intermediate 7 (100 mg, 0.230 mmol) and KF (40.1 mg, 0.690 mmol) in 1,4-dioxane (2.8 mL) and water (0.28 mL) was degassed with nitrogen for 15 min. Pd-118 (15.0 mg, 0.020 mmol) was added and the solution was degassed for a further 10 min before being left to stir overnight at room temperature. The reaction mixture was diluted with EtOAc (20 mL), washed with water (3×20 mL) and brine (20 mL). The organic extract was dried over MgSO₄, filtered and concentrated under reduced pressure to give the crude product, which was purified by flash silica column chromatography (gradient from 0-25% PE/EtOAc over 20 CV followed by a fast gradient to 100% EtOAc over 5 CV). The material was triturated in PE (5 mL) and stirred for 10 min. Once left to settle, the supernatant was removed and the process repeated. The resulting solid dried under reduced pressure to give N-(2-methoxy-6-thiazol-2-yl-3-pyridyl)-5-methyl-3-phenyl-isoxazole-4-carboxamide (43 mg, 45% yield) as a beige solid. ¹H NMR (500 MHZ, Chloroform-d) δ8.76 (d, J=8.1 Hz, 1H), 7.84 (d, J=3.2 Hz, 1H), 7.78 (s, 1H), 7.76 (d, J=8.2 Hz, 1H), 7.67-7.61 (m, 3H), 7.60-7.54 (m, 2H), 7.34 (d, J=3.2 Hz, 1H), 3.68 (s, 3H), 2.83 (s, 3H). m/z 393.1 [M+H]⁺.

### Example 67 N-[5-(2-Aminopyrimidin-5-yl)-3-methoxy-pyrazin-2-yl]-5-methyl-3-phenyl-isoxazole-4-carboxamide

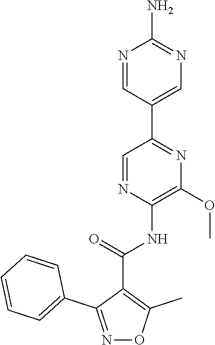 [SMILES: COc1nc(-c2cnc(N)nc2)cnc1NC(=O)c1c(-c2ccccc2)noc1C]

In a 10 mL vial was mixed Intermediate 6 (300 mg, 0.77 mmol), 5-(4,4,5,5-tetramethyl-1,3,2-dioxaborolan-2-yl)-2-pyrimidinamine (213 mg, 0.96 mmol) and KF (134 mg, 2.31 mmol) in 1,4-dioxane (2 mL) and water (1.5 mL). The mixture was degassed for 10 mins. In a separate vial was added Pd-118 (50 mg, 0.08 mmol) in 1,4-dioxane (2 mL) and the mixture was degassed for 10 mins. This was transferred to the main reaction vessel using a syringe and was stirred at room temperature overnight. The suspension was filtered, washed with water (10 mL) and diethyl ether (20 mL). The solid was dried under vacuum at 40° C. for 3 h to give N-[5-(2-aminopyrimidin-5-yl)-3-methoxy-pyrazin-2-yl]-5-methyl-3-phenyl-isoxazole-4-carboxamide (181 mg, 55% yield). ¹H NMR (500 MHZ, DMSO-d₆): δ10.54 (s, 1H), 8.92 (s, 2H), 8.47 (s, 1H), 7.64 (d, J=6.8 Hz, 2H), 7.42-7.53 (m, 3H), 7.10 (s, 2H), 3.96 (s, 3H), 2.61 (s, 3H). m/z 404.1 [M+H]⁺.

### Example 68 N-(6-Isoxazol-4-yl-2-methoxy-3-pyridyl)-5-methyl-3-phenyl-isoxazole-4-carboxamide

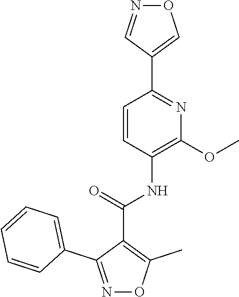 [SMILES: COc1nc(-c2cnoc2)ccc1NC(=O)c1c(-c2ccccc2)noc1C]

A 5 mL microwave vial was charged with Intermediate 1 (100 mg, 0.26 mmol), isoxazole-4-boronic acid (35 mg, 0.31 mmol), KOAc (101 mg, 1.03 mmol), Pd-118 (16.8 mg, 0.03 mmol), 1,4-dioxane (2 mL), and water (0.2 mL). The vial was sealed, purged with nitrogen and degassed before heated at 80° C. for 16 h. The reaction mixture was concentrated under reduced pressure. The residue was dissolved in DCM (25 mL) and washed with water (25 mL). The organic layer was concentrated under reduced pressure. The residue was adsorbed onto silica and purified by flash column chromatography (4 g silica, elution with 0-90% EtOAc/PE gradient) to afford N-(6-isoxazol-4-yl-2-methoxy-3-pyridyl)-5-methyl-3-phenyl-isoxazole-4-carboxamide (21 mg, 20% yield) as a brown solid. ¹H NMR (600 MHZ, Chloroform-d) δ8.80 (s, 1H), 8.69 (d, J=8.0 Hz, 1H), 8.65 (s, 1H), 7.74 (s, 1H), 7.63 (d, J=7.5 Hz, 3H), 7.58 (t, J=7.5 Hz, 2H), 7.05 (d, J=8.1 Hz, 1H), 3.65 (s, 3H), 2.83 (s, 3H). m/z 377.0 [M+H]⁺.

### Example 69 N-(6-Isothiazol-3-yl-2-methoxy-3-pyridyl)-5-methyl-3-phenyl-isoxazole-4-carboxamide

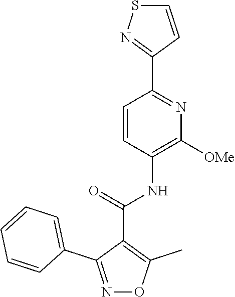 [SMILES: COc1nc(-c2ccsn2)ccc1NC(=O)c1c(-c2ccccc2)noc1C]

A solution of 3-bromoisothiazole (37.7 mg, 0.230 mmol), Intermediate 7 (100 mg, 0.230 mmol) and KF (40.1 mg, 0.690 mmol) in 1,4-dioxane (2.8 mL) and water (0.28 mL) was degassed with nitrogen for 15 min. Pd-118 (15.0 mg, 0.023 mmol) was added quickly, the solution degassed for a further 10 min before being left to stir at room temperature overnight. TLC analysis (3:1 PE:EtOAc) showed full consumption of the starting material and the presence of two new species (Rf=0.45 and 0.05). The reaction mixture was diluted with EtOAc (20 mL), washed with water (3×20 mL) and brine (20 mL). The organic extract was dried over MgSO₄, filtered and concentrated under reduced pressure to give the crude product, which was purified by flash silica column chromatography (gradient from 0-20% EtOAc/n-hexane over 15 CV followed by a fast gradient to 100% EtOAc) to give the desired product, which was further purified by trituration with PE (5 mL) and dried to give N-(6-isothiazol-3-yl-2-methoxy-3-pyridyl)-5-methyl-3-phenyl-isoxazole-4-carboxamide (11 mg, 12% yield) as a light brown solid. ¹H NMR (500 MHZ, Chloroform-d) δ8.76 (d, J=8.2 Hz, 1H), 8.65 (d, J=4.7 Hz, 1H), 7.89 (d, J=4.7 Hz, 1H), 7.81 (d, J=8.2 Hz, 1H), 7.79 (s, 1H), 7.63 (td, J=7.5, 1.4 Hz, 3H), 7.58 (dd, J=8.3, 6.3 Hz, 2H), 3.69 (s, 3H), 2.84 (s, 3H). m/z 393.1 [M+H]⁺.

### Example 70 Methyl 5-[6-methoxy-5-[(5-methyl-3-phenyl-isoxazole-4-carbonyl)amino]-2-pyridyl]pyrimidine-2-carboxylate

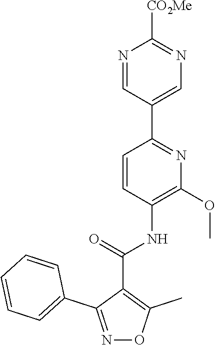 [SMILES: COC(=O)c1ncc(-c2ccc(NC(=O)c3c(-c4ccccc4)noc3C)c(OC)n2)cn1]

In a 25 mL microwave vial, Intermediate 7 (100 mg, 0.23 mmol), methyl 5-bromopyrimidine-2-carboxylate (60 mg, 0.28 mmol) and KF (40 mg, 0.69 mmol) were dissolved in 1,4-dioxane (4 mL) and water (1 mL) and the mixture was degassed for 10 mins. Pd-118 (15 mg, 0.02 mmol) was added to the mixture and the reaction mixture was stirred at room temperature overnight. Water (5 mL) was added to the reaction mixture and the solid was collected via vacuum filtration, washed with water (10 mL) and MeOH (10 mL) then dried in a vacuum oven (40° C.) for 3 h to give methyl 5-[6-methoxy-5-[(5-methyl-3-phenyl-isoxazole-4-carbonyl)amino]-2-pyridyl]pyrimidine-2-carboxylate (58 mg, 54% yield). ¹H NMR (500 MHZ, Chloroform-d) δ9.41 (s, 2H), 8.82 (d, J=8.1 Hz, 1H), 7.83 (s, 1H), 7.68-7.56 (m, 5H), 7.46 (d, J=8.1 Hz, 1H), 4.09 (s, 3H), 3.70 (s, 3H), 2.85 (s, 3H). m/z 446.0 [M+H]⁺.

### Example 71 N-[3-Methoxy-5-(6-methoxy-3-pyridyl) pyrazin-2-yl]-5-methyl-3-phenyl-isoxazole-4-carboxamide

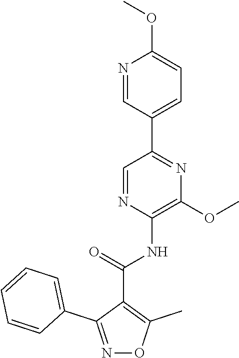 [SMILES: COc1ccc(-c2cnc(NC(=O)c3c(-c4ccccc4)noc3C)c(OC)n2)cn1]

A 5 mL microwave vial was charged with Intermediate 6 (100 mg, 0.26 mmol), KOAc (101 mg, 1.03 mmol), Pd-118 (16.8 mg, 0.03 mmol), (6-methoxypyridin-3-yl) boronic acid (47 mg, 0.31 mmol), 1,4-dioxane (2 mL) and water (0.2 mL). The vial was sealed, purged with nitrogen and degassed before heated at 80° C. for 16 h. The reaction mixtures were concentrated under reduced pressure. The resulting residue was dissolved in DCM (15 mL) and washed with water (15 mL), brine (15 mL), dried over MgSO₄, filtered and concentrated under reduced pressure. The residue was adsorbed onto silica and purified by flash column chromatography (4 g silica, elution with a 0-90% EtOAc/PE gradient) to afford N-[3-methoxy-5-(6-methoxy-3-pyridyl) pyrazin-2-yl]-5-methyl-3-phenyl-isoxazole-4-carboxamide (98 mg, 87% yield) as a yellow solid. ¹H NMR (600 MHZ, Chloroform-d) δ8.73 (s, 1H), 8.31 (s, 1H), 8.07 (d, J=8.9 Hz, 1H), 7.90 (s, 1H), 7.65-7.58 (m, 3H), 7.56 (t, J=7.5 Hz, 2H), 6.82 (d, J=8.7 Hz, 1H), 3.98 (s, 3H), 3.75 (s, 3H), 2.85 (s, 3H). m/z 418.0 [M+H]⁺.

### Example 72 N-[3-Methoxy-5-(2-methoxypyrimidin-5-yl) pyrazin-2-yl]-5-methyl-3-phenyl-isoxazole-4-carboxamide

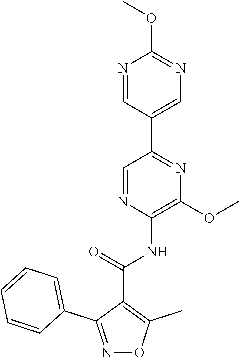 [SMILES: COc1ncc(-c2cnc(NC(=O)c3c(-c4ccccc4)noc3C)c(OC)n2)cn1]

A 5 mL microwave vial was charged with Intermediate 6 (100 mg, 0.26 mmol), KOAc (101 mg, 1.03 mmol), Pd-118 (16.8 mg, 0.03 mmol), (2-methoxy-5-pyrimidinyl) boronic acid (47 mg, 0.31 mmol), 1,4-dioxane (2 mL) and water (0.2 mL). The vial was sealed, purged with nitrogen and degassed before heated at 80° C. for 16 h. The reaction mixtures were concentrated under reduced pressure. The residue was dissolved in DCM (15 mL) and washed with water (15 mL), brine (15 mL), dried over MgSO₄, filtered and concentrated under reduced pressure. The residue was adsorbed onto silica and purified by flash column chromatography (4 g silica, elution with a 0-90% EtOAc/PE gradient) to afford N-[3-methoxy-5-(2-methoxypyrimidin-5-yl) pyrazin-2-yl]-5-methyl-3-phenyl-isoxazole-4-carboxamide (107 mg, 94% yield) as a yellow solid. ¹H NMR (600 MHZ, Chloroform-d) δ9.01 (s, 2H), 8.32 (s, 1H), 7.95 (s, 1H), 7.66-7.59 (m, 3H), 7.57 (t, J=7.4 Hz, 2H), 4.07 (s, 3H), 3.75 (s, 3H), 2.85 (s, 3H). m/z 419.0 [M+H]⁺.

### Example 73 N-[2-Methoxy-6-(2-pyridyl)-3-pyridyl]-5-methyl-3-phenyl-isoxazole-4-carboxamide

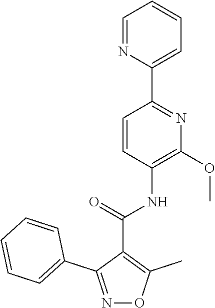 [SMILES: COc1nc(-c2ccccn2)ccc1NC(=O)c1c(-c2ccccc2)noc1C]

A solution of Intermediate 7 (100 mg, 0.26 mmol) and 2-bromopyridine (49 mg, 0.31 mmol) in 1,4-dioxane (4 mL) and water (1 mL) was sparged with nitrogen for 10 mins. The reaction was then charged with Pd-118 (17 mg, 0.03 mmol) and KF (45 mg, 0.77 mmol), vessel sealed and heated to 40° C. overnight. Temperature was increased to 80° C. for 4 h, then further increased to 120° C. for a further night. The reaction mixture was concentrated under reduced pressure and taken up in chloroform. Water was added and organics separated using a phase separator. The organics were removed under reduced pressure and residue purified by flash silica column chromatography on an ISCO system (20 g silica, elution with 0-50% EtOAc/PE gradient) to give N-[2-methoxy-6-(2-pyridyl)-3-pyridyl]-5-methyl-3-phenyl-isoxazole-4-carboxamide (32 mg, 31% yield). ¹H NMR (600 MHZ, Chloroform-d) δ8.78 (d, J=8.2 Hz, 1H), 8.62 (t, J=3.1 Hz, 1H), 8.23 (d, J=8.0 Hz, 1H), 8.02 (d, J=8.2 Hz, 1H), 7.80 (s, 1H), 7.74 (t, J=7.8 Hz, 1H), 7.67-7.61 (m, 3H), 7.58 (t, J=7.5 Hz, 2H), 7.23 (dd, J=7.1, 5.2 Hz, 1H), 3.72 (s, 3H), 2.84 (s, 3H). m/z 387.0 [M+H]⁺.

### Example 74 N-[6-[2-[(Dimethylamino)methyl]pyrimidin-5-yl]-2-methoxy-3-pyridyl]-5-methyl-3-phenyl-isoxazole-4-carboxamide

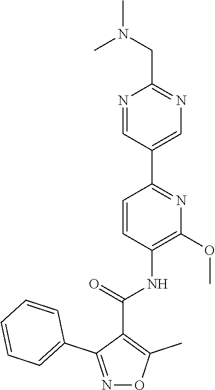 [SMILES: COc1nc(-c2cnc(CN(C)C)nc2)ccc1NC(=O)c1c(-c2ccccc2)noc1C]

### Step 1—Synthesis of Intermediate 8-5-Bromo-2-(bromomethyl)pyrimidine

A suspension of 5-bromo-2-methylpyrimidine (4.00 g, 23.12 mmol) and N-bromosuccinimide (5.20 g, 29.22 mmol) in benzotrifluoride (50 mL) had 2,2′-[(E)-1,2-diazenediyl] bis(2-methylpropanenitrile) (115 mg, 0.70 mmol) added. The mixture was heated to 80° C. for 24 h. The reaction mixture was concentrated under reduced pressure and purified by flash silica column chromatography on an ISCO system (80 g silica, elution with a 0-15% EtOAc/PE gradient) to give 5-bromo-2-(bromomethyl)pyrimidine (1.40 g, 24% yield). ¹H NMR (500 MHz, Chloroform-d): δ8.80 (s, 2H), 4.57 (s, 2H). m/z 252.8 [M+H]⁺ for ⁸¹Br.

### Step 2—Synthesis of Intermediate 9-1-(5-Bromopyrimidin-2-yl)-N,N-dimethyl-methanamine

A solution of Intermediate 8 (500 mg, 1.98 mmol) in THF (7 mL) was added 2M dimethylamine in THF (3.97 mL, 7.94 mmol) and the reaction mixture stirred at RT. The reaction formed a thick white precipitate almost immediately. After 2 h, suspension was evaporated to dryness and partitioned between EtOAc (30 mL) and 2M NaOH (10 mL). Organics were separated and aqueous extracted further with EtOAc (2×30 mL). The organics were dried over MgSO₄, filtered and concentrated to give 1-(5-bromopyrimidin-2-yl)-N,N-dimethyl-methanamine (450 mg, 100% yield). ¹H NMR (500 MHZ, Chloroform-d) δ8.71 (s, 2H), 3.65 (s, 2H), 2.29 (s, 6H). m/z 216.0 [M+H]⁺.

### Step 3

A mixture of Intermediate 7 (900 mg, 2.07 mmol), Intermediate 9 (450 mg, 2.08 mmol) and ground KF (360 mg, 6.2 mmol) in 1,4-dioxane (10 mL) and water (5 mL) was sparged with nitrogen for 10 mins. Pd-118 (135 mg, 0.21 mmol) was added and mixture heated to 45° C. overnight. 1,4-dioxane was removed under reduced pressure and the mixture partitioned between DCM (20 mL) and brine (10 mL). The mixture was passed through phase separator and the aqueous phase further extracted with DCM (2×20 mL). The organics were combined and concentrated to dryness and residue purified by flash silica column chromatography on an ISCO system (24 g silica, elution with a 0-20% MeOH/EtOAc gradient). The resultant crude solid was re-crystallised from minimum amount isopropanol (˜30 mL). The solid was cooled in fridge for 2 h before filtration to give N-[6-[2-[(dimethylamino)methyl]pyrimidin-5-yl]-2-methoxy-3-pyridyl]-5-methyl-3-phenyl-isoxazole-4-carboxamide (280 mg, 0.60 mmol, 29% yield). ¹H NMR (500 MHz, Chloroform-d) δ9.23 (s, 2H), 8.78 (d, J=8.1 Hz, 1H), 7.80 (s, 1H), 7.68-7.56 (m, 5H), 7.37 (d, J=8.1 Hz, 1H), 3.79 (s, 2H), 3.70 (s, 3H), 2.85 (s, 3H), 2.39 (s, 6H). m/z 445.1 [M+H]⁺.

### Example 75 N-[2-Methoxy-6-[2-(methylcarbamoyl)pyrimidin-5-yl]-3-pyridyl]-5-methyl-3-phenyl-isoxazole-4-carboxamide

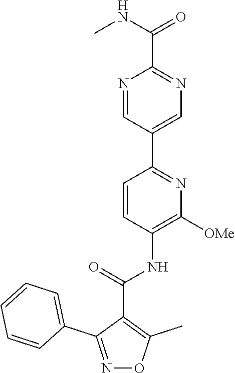 [SMILES: CNC(=O)c1ncc(-c2ccc(NC(=O)c3c(-c4ccccc4)noc3C)c(OC)n2)cn1]

### Step 1

5-Bromo-2-pyrimidinecarboxylic acid (300 mg, 1.48 mmol) and SOCl₂ (2 mL, 27.42 mmol) were heated in a sealed 20 mL microwave vial overnight at 65° C. The mixture was left to cool to room temperature and chloroform (2 mL) was added followed by the drop-wise addition of DMF (3 drops). The mixture was then reheated to 80° C. for 2 h. The volatiles were removed under reduced pressure and the solid re-dissolved in DCM (5 mL). The solution was cooled using an ice bath and a 2M solution of methylamine in THF (2 mL, 4.0 mmol) was added drop-wise. Upon completion of addition, the cooling bath was removed and the mixture left to stir for 3.5 h at room temperature. Water (10 mL) was added along with additional DCM (20 mL). The layers were separated, the aqueous layer twice extracted with DCM (2×15 mL). The combined organics were washed with 2 M NaOH solution (2×20 mL), dried over MgSO₄, filtered and concentrated under reduced pressure to give 5-bromo-N-methyl-pyrimidine-2-carboxamide (277 mg, 86% yield) as a white crystalline solid. ¹H NMR (500 MHZ, Chloroform-d) δ8.87 (s, 2H), 7.89 (s, 1H), 3.03 (d, J=5.1 Hz, 3H). m/z 217.9 [M+H]⁺ for ⁸¹Br.

### Step 2

A solution of KF (76.1 mg, 1.31 mmol), 5-bromo-N-methyl-pyrimidine-2-carboxamide (99.3 mg, 0.459 mmol) and Intermediate 7 (200 mg, 0.46 mmol) in 1,4-dioxane (3.4 mL) and water (0.8 mL) was degassed with nitrogen for 15 min. Pd-118 (28.5 mg, 0.044 mmol) was added, the solution degassed again for 10 min and the mixture left to stir at room temperature overnight. The reaction was heated to 45° C. for 5 h to drive reaction completion. The reaction mixture was diluted with EtOAc (20 mL), washed with water (20 mL), the aqueous phase back extracted with DCM (3×20 mL) and the combined organic extracts dried over MgSO₄, filtered and concentrated under reduced pressure to give the crude product, which was purified by ISCO column chromatography (12 g silica, gradient from 0-10% MeOH/DCM) to give partially pure product, which was further purified by trituration in MeOH (20 mL), the supernatant removed by pipette and the solid concentrated under reduced pressure to give N-[2-methoxy-6-[2-(methylcarbamoyl)pyrimidin-5-yl]-3-pyridyl]-5-methyl-3-phenyl-isoxazole-4-carboxamide (111 mg, 54% yield) as a beige solid. ¹H NMR (500 MHZ, Chloroform-d) δ9.34 (s, 2H), 8.81 (d, J=8.1 Hz, 1H), 8.02 (d, J=5.6 Hz, 1H), 7.82 (s, 1H), 7.69-7.62 (m, 3H), 7.63-7.56 (m, 2H), 7.44 (d, J=8.1 Hz, 1H), 3.71 (s, 3H), 3.10 (d, J=5.1 Hz, 3H), 2.85 (s, 3H). m/z 445.1 [M+H]⁺.

### Example 76 N-(6-Methoxy-6′-((trifluoromethyl)thio)-[2,3′-bipyridin]-5-yl)-5-methyl-3-phenyl-isoxazole-4-carboxamide

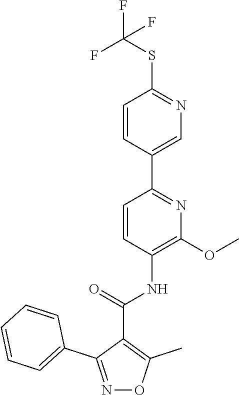 [SMILES: COc1nc(-c2ccc(SC(F)(F)F)nc2)ccc1NC(=O)c1c(-c2ccccc2)noc1C]

By way of method described for Example 41, substituting (6-((trifluoromethyl)thio) pyridin-3-yl) boronic acid for (2-methoxypyrimidin-5-yl) boronic acid (33 mg, 16% yield). ¹H NMR (500 MHz, DMSO-d₆) δ9.52 (s, 1H), 9.33 (d, J=2.4 Hz, 1H), 8.53 (dd, J=8.2, 2.4 Hz, 1H), 8.44 (s, 1H), 7.84 (d, J=8.2 Hz, 1H), 7.78 (d, J=8.1 Hz, 1H), 7.73-7.68 (m, 2H), 7.61-7.51 (m, 3H), 3.92 (s, 3H), 2.67 (s, 3H). m/z 487.0 [M+H]⁺.

### Example 77 N-(2-Methoxy-6-(3-methyl-2-oxo-2,3-dihydro-1H-benzo[d]imidazol-5-yl)pyridin-3-yl)-5-methyl-3-phenylisoxazole-4-carboxamide

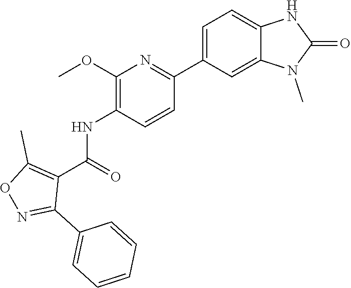 [SMILES: COc1nc(-c2ccc3[nH]c(=O)n(C)c3c2)ccc1NC(=O)c1c(-c2ccccc2)noc1C]

By way of method described for Example 41, substituting (3-methyl-2-oxo-2,3-dihydro-1H-benzo[d] imidazol-5-yl) boronic acid for (2-methoxypyrimidin-5-yl) boronic acid (192 mg, 76% yield). ¹H NMR (500 MHZ, DMSO-d₆) δ10.97 (s, 1H), 9.44 (s, 1H), 8.30 (d, J=8.2 Hz, 1H), 7.81-7.75 (m, 2H), 7.75-7.70 (m, 2H), 7.60-7.55 (m, 4H), 7.05 (d, J=8.1 Hz, 1H), 3.93 (s, 3H), 3.35 (s, 3H), 2.67 (s, 3H). m/z 456.1 [M+H]⁺.

### Example 78 N-(4′-Amino-6-methoxy-[2,3′-bipyridin]-5-yl)-5-methyl-3-phenylisoxazole-4-carboxamide

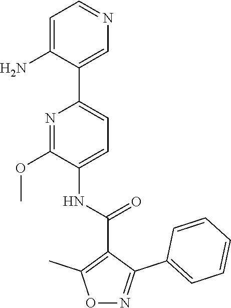 [SMILES: COc1nc(-c2cnccc2N)ccc1NC(=O)c1c(-c2ccccc2)noc1C]

By way of method described for Example 41, substituting (4-aminopyridin-3-yl) boronic acid for (2-methoxypyrimidin-5-yl) boronic acid (40 mg, 19% yield). ¹H NMR (500 MHZ, DMSO-d₆) δ9.55 (s, 1H), 8.46 (s, 1H), 8.35 (d, J=8.2 Hz, 1H), 7.99 (d, J=5.6 Hz, 1H), 7.75-7.70 (m, 2H), 7.60-7.51 (m, 3H), 7.43 (d, J=8.2 Hz, 1H), 6.90 (s, 2H), 6.67 (d, J=5.6 Hz, 1H), 3.86 (s, 3H), 2.66 (s, 3H). m/z 402.2 [M+H]⁺.

### Example 79 N-[6-(6,8-Dihydro-5H-imidazo[2,1-c][1,4]oxazin-3-yl)-2-methoxy-3-pyridyl]-5-methyl-3-phenyl-isoxazole-4-carboxamide

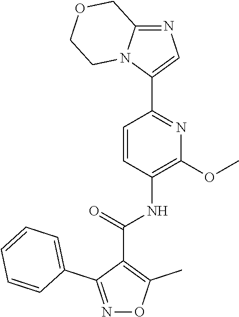 [SMILES: COc1nc(-c2cnc3n2CCOC3)ccc1NC(=O)c1c(-c2ccccc2)noc1C]

### Step 1

In 5 mL microwave vial a solution of 5H,6H,8H-imidazo[2,1-c][1,4]oxazine (500 mg, 4.03 mmol) in MeCN (2 mL) was treated at 0° C. a suspension of N-bromosuccinimide (717 mg, 4.03 mmol) in MeCN (1 mL) and the reaction was stirred at 0° C. for 30 min. The reaction mixture was concentrated under reduced pressure and the residue was redissolved in EtOAc (10 mL) and washed with water (3×10 mL), brine (10 mL), dried over MgSO₄, filtered and concentrated under reduced pressure. The mass recovery was low; therefore, all aqueous solutions were combined and extracted with EtOAc (2×25 mL). The combined organic extracts were dried over MgSO₄, filtered and concentrated with previously extracted compound under reduced pressure to afford 3-bromo-6,8-dihydro-5H-imidazo[2,1-c][1,4]oxazine (599 mg) as an off-white solid. The crude solid (90% pure) was used directly in the next step. ¹H NMR (500 MHZ, Chloroform-d) δ6.98 (s, 1H), 4.79 (s, 2H), 4.11-4.05 (m, 2H), 3.94-3.83 (m, 2H). m/z 202.9 [M+H, ⁷⁹Br]⁺, 204.9 [M+H, ⁸¹Br]⁺.

### Step 2

A 25 mL microwave vial was charged with Intermediate 7 (687 mg, 1.50 mmol), crude 3-bromo-5,6-dihydro-8H-imidazo[2,1-c][1,4]oxazine (406 mg, 1.80 mmol), KF (261 mg, 4.50 mmol), 1,4-dioxane (15 mL) and water (5 mL). The mixture was degassed for 10 min before adding Pd-118 (196 mg, 0.30 mmol). The vial was sealed and heated at 50° C. for 16 h. Another portion of Pd-118 (98 mg, 0.15 mmol) was added and the reaction mixture was heated at 50° C. for 1 h. Water (10 mL) was added and the solution was extracted with EtOAc (3×10 mL). The combined organic extracts were concentrated under reduced pressure.

The residue was adsorbed onto silica and purified by flash column chromatography (12 g silica, elution with a 0-10% MeOH/EtOAc gradient) to afford a yellow solid, which was triturated in EtOAc then PE to afford N-[6-(6,8-dihydro-5H-imidazo[2,1-c][1,4]oxazin-3-yl)-2-methoxy-3-pyridyl]-5-methyl-3-phenyl-isoxazole-4-carboxamide (83 mg, 12% yield) as a brown solid. ¹H NMR (500 MHZ, Chloroform-d) δ8.66 (d, J=8.2 Hz, 1H), 7.68 (s, 1H), 7.66-7.53 (m, 5H), 7.36 (s, 1H), 7.15 (d, J=8.2 Hz, 1H), 4.90 (s, 2H), 4.42 (t, J=5.3 Hz, 2H), 4.04 (t, J=5.2 Hz, 2H), 3.61 (s, 3H), 2.83 (s, 3H). m/z 432.0 [M+H]⁺.

### Example 80 N-(6-(1-Ethyl-3-(thiazol-2-yl)-1H-pyrazol-5-yl)-2-methoxypyridin-3-yl)-5-methyl-3-phenylisoxazole-4-carboxamide

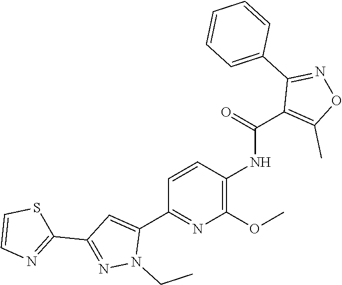 [SMILES: CCn1nc(-c2nccs2)cc1-c1ccc(NC(=O)c2c(-c3ccccc3)noc2C)c(OC)n1]

By way of method described for Example 41, substituting (1-ethyl-3-(thiazol-2-yl)-1H-pyrazol-5-yl) boronic acid for (2-methoxypyrimidin-5-yl) boronic acid (28 mg, 14% yield). ¹H NMR (500 MHZ, DMSO-d₆) δ9.48 (s, 1H), 8.40 (s, 1H), 8.37-8.32 (m, 1H), 7.90 (d, J=2.3 Hz, 1H), 7.74-7.66 (m, 2H), 7.61 (d, J=8.1 Hz, 1H), 7.59-7.49 (m, 3H), 6.76 (d, J=2.3 Hz, 1H), 4.22 (q, J=7.3 Hz, 2H), 3.87 (s, 3H), 2.65 (s, 3H), 1.42 (t, J=7.3 Hz, 3H). m/z 487.1 [M+H]⁺.

### Example 81 N-(2′-Amino-6-methoxy-6′-methyl-[2,4′-bipyridin]-5-yl)-5-methyl-3-phenylisoxazole-4-carboxamide

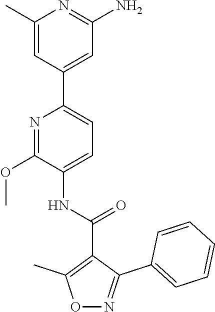 [SMILES: COc1nc(-c2cc(C)nc(N)c2)ccc1NC(=O)c1c(-c2ccccc2)noc1C]

By way of method described for Example 41, substituting (2-amino-6-methylpyridin-4-yl) boronic acid for (2-methoxypyrimidin-5-yl) boronic acid (44 mg, 22% yield). ¹H NMR (500 MHz, DMSO-d₆) δ9.47 (s, 1H), 8.39 (d, J=7.7 Hz, 1H), 7.73-7.68 (m, 2H), 7.60-7.47 (m, 4H), 6.97 (d, J=1.4 Hz, 1H), 6.91 (s, 1H), 5.91 (s, 2H), 3.90 (s, 3H), 2.66 (s, 3H), 2.30 (s, 3H). m/z 416.2 [M+H]⁺.

### Example 82 N-(2′-(Dimethylamino)-6-methoxy-[2,4′-bipyridin]-5-yl)-5-methyl-3-phenyl-isoxazole-4-carboxamide

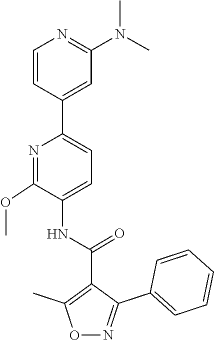 [SMILES: COc1nc(-c2ccnc(N(C)C)c2)ccc1NC(=O)c1c(-c2ccccc2)noc1C]

By way of method described for Example 41, substituting (2-(dimethylamino)pyridin-4-yl) boronic acid for (2-methoxypyrimidin-5-yl) boronic acid (58 mg, 27% yield). ¹H NMR (500 MHz, DMSO-d₆) δ9.47 (s, 1H), 8.40 (s, 1H), 8.16 (d, J=5.2 Hz, 1H), 7.74-7.68 (m, 3H), 7.61-7.52 (m, 3H), 7.23 (s, 1H), 7.20 (dd, J=5.2, 1.4 Hz, 1H), 3.90 (s, 3H), 3.09 (s, 6H), 2.67 (s, 3H). m/z 430.2 [M+H]⁺.

### Example 83 N-(2-Methoxy-6-(pyrazolo[1,5-b]pyridazin-3-yl)pyridin-3-yl)-5-methyl-3-phenylisoxazole-4-carboxamide

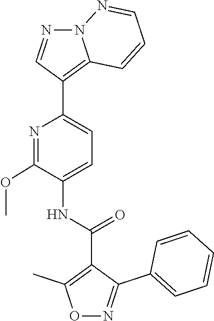 [SMILES: COc1nc(-c2cnn3ncccc23)ccc1NC(=O)c1c(-c2ccccc2)noc1C]

By way of method described for Example 41, substituting pyrazolo[1,5-b]pyridazin-3-ylboronic acid for (2-methoxypyrimidin-5-yl) boronic acid (58 mg, 34% yield). ¹H NMR (500 MHz, DMSO-d₆) δ9.49 (s, 1H), 8.94 (dd, J=9.0, 1.9 Hz, 1H), 8.73 (s, 1H), 8.55-8.48 (m, 1H), 8.29 (d, J=8.1 Hz, 1H), 7.76-7.71 (m, 2H), 7.60-7.51 (m, 4H), 7.39-7.30 (m, 1H), 3.97 (s, 3H), 2.67 (s, 3H). m/z 427.2 [M+H]⁺.

### Example 84 N-(2-Methoxy-6-(2-(tetrahydro-2H-pyran-2-yl)-2H-1,2,3-triazol-4-yl)pyridin-3-yl)-5-methyl-3-phenylisoxazole-4-carboxamide

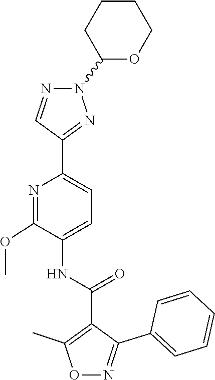

By way of method described for Example 41, substituting 2-(oxan-2-yl)-4-(tetramethyl-1,3,2-dioxaborolan-2-yl)-2H-1,2,3-triazole (42 mg, 21% yield). ¹H NMR (500 MHZ, DMSO-d₆) δ9.47 (s, 1H), 8.40 (d, J=7.9 Hz, 1H), 8.27 (s, 1H), 7.74-7.68 (m, 2H), 7.61-7.46 (m, 4H), 5.79 (dd, J=9.4, 2.5 Hz, 1H), 3.94-3.82 (m, 4H), 3.73-3.68 (m, 1H), 2.66 (s, 3H), 2.35-2.24 (m, 1H), 2.07-1.98 (m, 2H), 1.75-1.71 (m, 1H), 1.63-1.55 (m, 2H). m/z 461.1 [M+H]⁺.

### Example 85 N-(6-(2-(4-Hydroxypiperidin-1-yl)pyrimidin-5-yl)-2-methoxypyridin-3-yl)-5-methyl-3-phenylisoxazole-4-carboxamide

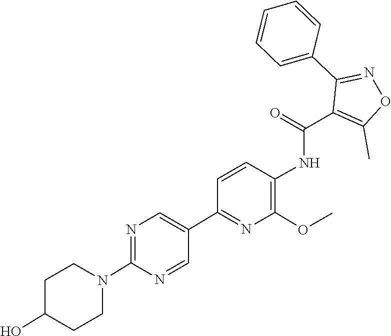 [SMILES: COc1nc(-c2cnc(N3CCC(O)CC3)nc2)ccc1NC(=O)c1c(-c2ccccc2)noc1C]

By way of method described for Example 41, substituting 1-[5-(tetramethyl-1,3,2-dioxaborolan-2-yl)pyrimidin-2-yl]piperidin-4-ol for (2-methoxypyrimidin-5-yl) boronic acid (218 mg, 87% yield). ¹H NMR (500 MHZ, DMSO-d₆) δ9.45 (s, 1H), 9.00 (s, 2H), 8.27 (s, 1H), 7.74-7.69 (m, 2H), 7.57-7.52 (m, 3H), 7.52-7.47 (m, 1H), 4.77-4.71 (m, 1H), 4.36-4.28 (m, 2H), 3.88 (s, 3H), 3.80-3.73 (m, 1H), 2.66 (s, 3H), 1.83-1.77 (m, 2H), 1.40-1.30 (m, 2H). CH₂ under residual solvent. m/z 487.2 [M+H]⁺.

### Example 86 N-(3′-Amino-6-methoxy-[2,4′-bipyridin]-5-yl)-5-methyl-3-phenylisoxazole-4-carboxamide

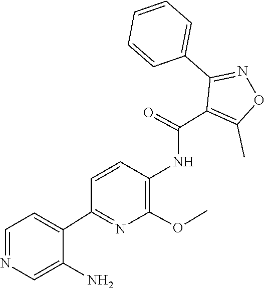 [SMILES: COc1nc(-c2ccncc2N)ccc1NC(=O)c1c(-c2ccccc2)noc1C]

By way of method described for Example 41, substituting (3-aminopyridin-4-yl) boronic acid for (2-methoxypyrimidin-5-yl) boronic acid (80 mg, 39% yield). ¹H NMR (500 MHZ, DMSO-d₆) δ9.56 (s, 1H), 8.41 (s, 1H), 8.14 (s, 1H), 7.80 (d, J=5.2 Hz, 1H), 7.74-7.69 (m, 2H), 7.60-7.51 (m, 4H), 7.44 (d, J=5.2 Hz, 1H), 6.36 (s, 2H), 3.86 (s, 3H), 2.66 (s, 3H). m/z 402.2 [M+H]⁺.

### Example 87 N-(5′-(Difluoromethoxy)-6-methoxy-[2,3′-bipyridin]-5-yl)-5-methyl-3-phenylisoxazole-4-carboxamide

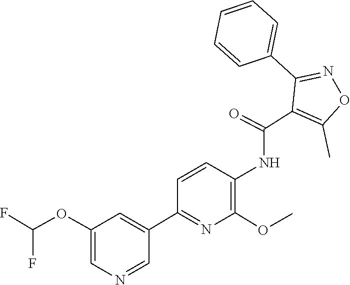 [SMILES: COc1nc(-c2cncc(OC(F)F)c2)ccc1NC(=O)c1c(-c2ccccc2)noc1C]

By way of method described for Example 41, substituting 3-(difluoromethoxy)-5-(tetramethyl-1,3,2-dioxaborolan-2-yl)pyridine for (2-methoxypyrimidin-5-yl) boronic acid (221 mg, 88% yield). ¹H NMR (500 MHZ, DMSO-d₆) δ9.51 (s, 1H), 9.19-9.15 (m, 1H), 8.51 (d, J=2.7 Hz, 1H), 8.43 (s, 1H), 8.23 (s, 1H), 7.78 (d, J=8.1 Hz, 1H), 7.71 (d, J=7.0 Hz, 2H), 7.62-7.52 (m, 3H), 7.44 (t, J=73.8 Hz, 1H) 3.92 (s, 3H), 2.67 (s, 3H). m/z 453.1 [M+H]⁺.

### Example 88 N-(6′-(Difluoromethoxy)-6-methoxy-[2,3′-bipyridin]-5-yl)-5-methyl-3-phenyl-isoxazole-4-carboxamide

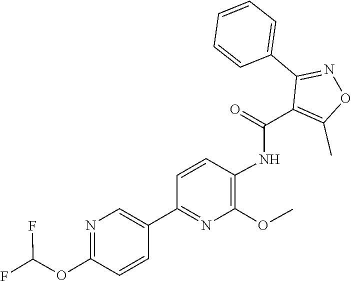 [SMILES: COc1nc(-c2ccc(OC(F)F)nc2)ccc1NC(=O)c1c(-c2ccccc2)noc1C]

By way of method described for Example 41, substituting 2-(difluoromethoxy)-5-(tetramethyl-1,3,2-dioxaborolan-2-yl)pyridine for (2-methoxypyrimidin-5-yl) boronic acid (13 mg, 6% yield). ¹H NMR (500 MHZ, DMSO-d₆) δ9.50 (s, 1H), 8.94 (d, J=2.5 Hz, 1H), 8.54 (dd, J=8.6, 2.5 Hz, 1H), 8.39 (d, J=10.9 Hz, 1H), 7.76 (t, J=74.5 Hz, 1H), 7.74-7.69 (m, 2H), 7.69-7.64 (m, 1H), 7.59-7.50 (m, 3H), 7.20 (d, J=8.6 Hz, 1H), 3.91 (s, 3H), 2.66 (s, 3H). m/z 453.1 [M+H]⁺.

### Example 89 N-(6′-Amino-6-methoxy-[2,3′-bipyridin]-5-yl)-5-methyl-3-phenylisoxazole-4-carboxamide

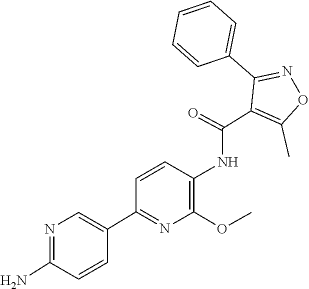 [SMILES: COc1nc(-c2ccc(N)nc2)ccc1NC(=O)c1c(-c2ccccc2)noc1C]

By way of method described for Example 41, substituting (6-aminopyridin-3-yl) boronic acid for (2-methoxypyrimidin-5-yl) boronic acid (15 mg, 8% yield). ¹H NMR (500 MHZ, DMSO-d₆) δ9.42 (s, 1H), 8.64 (d, J=2.4 Hz, 1H), 8.23 (d, J=7.9 Hz, 1H), 8.03 (dd, J=8.7, 2.5 Hz, 1H), 7.74-7.69 (m, 2H), 7.60-7.51 (m, 3H), 7.41 (d, J=8.1 Hz, 1H), 6.51 (d, J=8.7 Hz, 1H), 6.25-6.20 (m, 2H), 3.88 (s, 3H), 2.66 (s, 3H). m/z 402.2 [M+H]⁺.

### Example 90 N-(6-Methoxy-5′-(1H-pyrazol-1-yl)-[2,3′-bipyridin]-5-yl)-5-methyl-3-phenylisoxazole-4-carboxamide

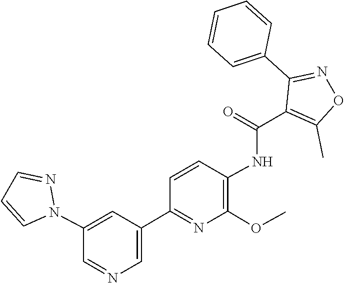 [SMILES: COc1nc(-c2cncc(-n3cccn3)c2)ccc1NC(=O)c1c(-c2ccccc2)noc1C]

By way of method described for Example 41, substituting (2-methoxypyrimidin-5-yl) boronic acid with (5-(1H-pyrazol-1-yl)pyridin-3-yl) boronic acid (55 mg, 27% yield). ¹H NMR (500 MHz, DMSO-d₆) δ9.53 (s, 1H), 9.21 (d, J=2.0 Hz, 1H), 9.13 (d, J=2.4 Hz, 1H), 8.79-8.75 (m, 1H), 8.73 (d, J=2.6 Hz, 1H), 8.46 (s, 1H), 7.87 (d, J=1.7 Hz, 1H), 7.84 (d, J=8.1 Hz, 1H), 7.72 (d, J=6.9 Hz, 2H), 7.62-7.52 (m, 3H), 6.67-6.63 (m, 1H), 3.95 (s, 3H), 2.68 (s, 3H). m/z 453.2 [M+H]⁺.

### Example 91 N-(2-Methoxy-6-(1-methyl-1H-benzo[d] [1,2,3]triazol-6-yl)pyridin-3-yl)-5-methyl-3-phenylisoxazole-4-carboxamide

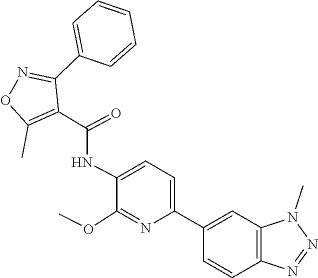 [SMILES: COc1nc(-c2ccc3nnn(C)c3c2)ccc1NC(=O)c1c(-c2ccccc2)noc1C]

By way of method described for Example 41, substituting (2-methoxypyrimidin-5-yl) boronic acid with 1-methyl-6-(tetramethyl-1,3,2-dioxaborolan-2-yl)-1H-1,2,3-benzotriazole (57 mg, 29% yield). ¹H NMR (500 MHZ, DMSO-d₆) δ9.50 (s, 1H), 8.50 (s, 1H), 8.44 (s, 1H), 8.17 (dd, J=8.7, 1.5 Hz, 1H), 8.09 (d, J=8.8 Hz, 1H), 7.79 (d, J=8.1 Hz, 1H), 7.72 (dd, J=6.8, 1.4 Hz, 2H), 7.62-7.52 (m, 3H), 4.38 (s, 3H), 3.97 (s, 3H), 2.68 (s, 3H). m/z 441.2 [M+H]⁺.

### Example 92 N-(6-(2-(3-(Hydroxymethyl) piperidin-1-yl)pyrimidin-5-yl)-2-methoxypyridin-3-yl)-5-methyl-3-phenylisoxazole-4-carboxamide

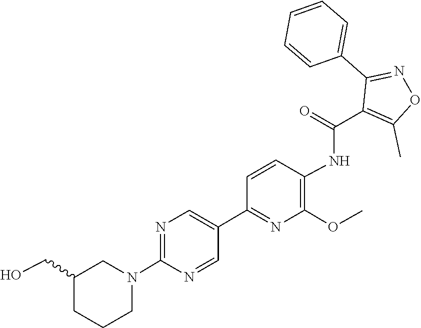 [SMILES: COc1nc(-c2cnc(N3CCCC(CO)C3)nc2)ccc1NC(=O)c1c(-c2ccccc2)noc1C]

By way of method described for Example 41, substituting (2-methoxypyrimidin-5-yl) boronic acid with {1-[5-(tetramethyl-1,3,2-dioxaborolan-2-yl)pyrimidin-2-yl]piperidin-3-yl}methanol (32 mg, 16% yield). ¹H NMR (500 MHz, DMSO-d₆) δ9.46 (s, 1H), 8.99 (s, 2H), 8.26 (d, J=7.8 Hz, 1H), 7.74-7.69 (m, 2H), 7.60-7.51 (m, 3H), 7.49 (d, J=8.1 Hz, 1H), 4.75 (dd, J=13.0, 3.9 Hz, 1H), 4.61 (d, J=13.0 Hz, 1H), 4.57 (t, J=5.3 Hz, 1H), 3.89 (s, 3H), 3.40-3.24 (m, 2H), 2.95 (dt, J=10.1, 2.7 Hz, 1H), 2.72 (dd, J=13.0, 10.6 Hz, 1H), 2.66 (s, 3H), 1.79-1.68 (m, 2H), 1.62-1.54 (m, 1H), 1.41 (tdd, J=12.1, 8.3, 3.8 Hz, 1H), 1.24 (tdd, J=11.9, 11.5, 3.5 Hz, 1H). m/z 501.2 [M+H]⁺.

### Example 93 N-(2-Methoxy-6-(4-methoxypyrimidin-5-yl)pyridin-3-yl)-5-methyl-3-phenyl-isoxazole-4-carboxamide

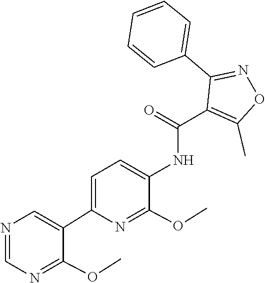 [SMILES: COc1nc(-c2cncnc2OC)ccc1NC(=O)c1c(-c2ccccc2)noc1C]

By way of method described for Example 41, substituting (2-methoxypyrimidin-5-yl) boronic acid with 4-methoxy-5-(tetramethyl-1,3,2-dioxaborolan-2-yl)pyrimidine (63 mg, 32% yield). ¹H NMR (500 MHZ, DMSO-d₆) δ9.50 (s, 1H), 9.15 (s, 1H), 8.81 (s, 1H), 8.38 (s, 1H), 7.73-7.67 (m, 3H), 7.61-7.51 (m, 3H), 4.07 (s, 3H), 3.89 (s, 3H), 2.67 (s, 3H). m/z 418.2 [M+H]⁺.

### Example 94 N-(2-Methoxy-6-(thiazol-5-yl)pyridin-3-yl)-5-methyl-3-phenylisoxazole-4-carboxamide

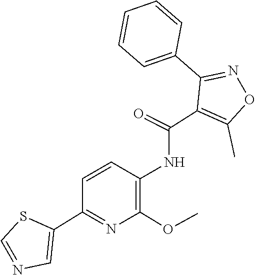 [SMILES: COc1nc(-c2cncs2)ccc1NC(=O)c1c(-c2ccccc2)noc1C]

By way of method described for Example 41, substituting (2-methoxypyrimidin-5-yl) boronic acid with (2-methoxy-6-(thiazol-5-yl)pyridin-3-yl) boronic acid (33 mg, 21% yield). ¹H NMR (500 MHZ, DMSO-d₆) δ9.60-9.45 (m, 1H), 9.10 (s, 1H), 8.50 (s, 1H), 8.35 (s, 1H), 7.70 (d, J=7.5 Hz, 2H), 7.63-7.45 (m, 4H), 3.85 (s, 3H), 2.66 (s, 3H). m/z 393.1 [M+H]⁺.

### Example 95 N-(5′-(Dimethylcarbamoyl)-6-methoxy-[2,3′-bipyridin]-5-yl)-5-methyl-3-phenyl-isoxazole-4-carboxamide

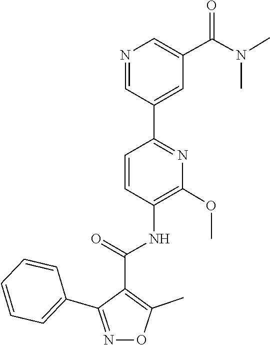 [SMILES: COc1nc(-c2cncc(C(=O)N(C)C)c2)ccc1NC(=O)c1c(-c2ccccc2)noc1C]

By way of method described for Example 41, substituting (2-methoxypyrimidin-5-yl) boronic acid with N,N-Dimethyl-5-(4,4,5,5-tetramethyl-1,3,2-dioxaborolan-2-yl)-3-pyridinecarboxamide (106 mg, 49% yield). ¹H NMR (600 MHZ, Chloroform-d) δ9.21 (d, J=2.1 Hz, 1H), 8.76 (d, J=8.1 Hz, 1H), 8.61 (d, J=1.9 Hz, 1H), 8.30 (d, J=2.2 Hz, 1H), 7.79 (s, 1H), 7.68-7.60 (m, 3H), 7.60-7.55 (m, 2H), 7.39 (d, J=8.2 Hz, 1H), 3.70 (s, 3H), 3.16 (s, 3H), 3.04 (s, 3H), 2.84 (s, 3H). m/z 458.0 [M+H]⁺.

### Example 96 N-(6-([1,2,4] Triazolo[4,3-a]pyridin-6-yl)-2-methoxypyridin-3-yl)-5-methyl-3-phenylisoxazole-4-carboxamide

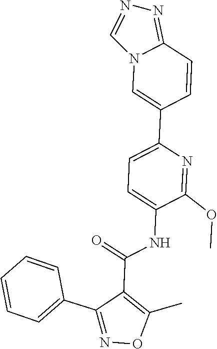 [SMILES: COc1nc(-c2ccc3nncn3c2)ccc1NC(=O)c1c(-c2ccccc2)noc1C]

By way of method described for Example 41, substituting (2-methoxypyrimidin-5-yl) boronic acid with [1,2,4]triazolo[4,3-a]pyridin-6-ylboronic acid (81 mg, 32% yield). ¹H NMR (600 MHz, Chloroform-d) δ8.87 (s, 1H), 8.77 (d, J=8.2 Hz, 1H), 8.75 (s, 1H), 7.87-7.76 (m, 3H), 7.69-7.61 (m, 3H), 7.60-7.55 (m, 2H), 7.31 (d, J=8.2 Hz, 1H), 3.72 (s, 3H), 2.84 (s, 3H). m/z 427.0 [M+H]⁺.

### Example 97 N-(2-Methoxy-6-(1-oxoisoindolin-4-yl)pyridin-3-yl)-5-methyl-3-phenylisoxazole-4-carboxamide

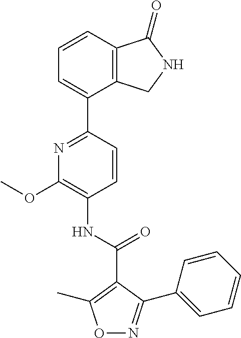 [SMILES: COc1nc(-c2cccc3c2CNC3=O)ccc1NC(=O)c1c(-c2ccccc2)noc1C]

By way of method described for Example 41, substituting (2-methoxypyrimidin-5-yl) boronic acid with 4-(tetramethyl-1,3,2-dioxaborolan-2-yl)-2,3-dihydro-1H-isoindol-1-one (55 mg, 28% yield). ¹H NMR (500 MHZ, DMSO-d₆) δ9.53 (s, 1H), 8.69 (s, 1H), 8.43 (br s, 1H), 8.16 (d, J=7.8 Hz, 1H), 7.75-7.64 (m, 4H), 7.62-7.48 (m, 4H), 4.84 (s, 2H), 3.92 (s, 3H), 2.67 (s, 3H). m/z 441.1 [M+H]⁺.

### Example 98 N-(5′-Chloro-6′-(dimethylamino)-6-methoxy-[2,3′-bipyridin]-5-yl)-5-methyl-3-phenylisoxazole-4-carboxamide

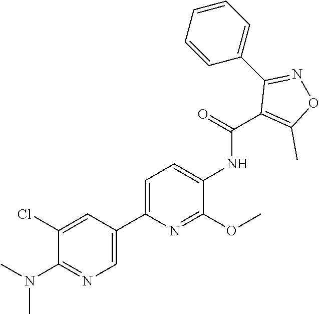 [SMILES: COc1nc(-c2cnc(N(C)C)c(Cl)c2)ccc1NC(=O)c1c(-c2ccccc2)noc1C]

By way of method described for Example 41, substituting (2-methoxypyrimidin-5-yl) boronic acid with (5-chloro-6-(dimethylamino)pyridin-3-yl) boronic acid (186 mg, 76% yield). ¹H NMR (500 MHZ, DMSO-d₆) δ9.47 (s, 1H), 8.87-8.83 (m, 1H), 8.35-8.29 (m, 2H), 7.71 (d, J=6.9 Hz, 2H), 7.60 (d, J=8.4 Hz, 1H), 7.58-7.51 (m, 3H), 3.90 (s, 3H), 3.01 (s, 6H), 2.66 (s, 3H). m/z 464.1 [M+H]⁺.

### Example 99 (R)—N-(6′-(3-Hydroxypyrrolidin-1-yl)-6-methoxy-[2,3′-bipyridin]-5-yl)-5-methyl-3-phenylisoxazole-4-carboxamide

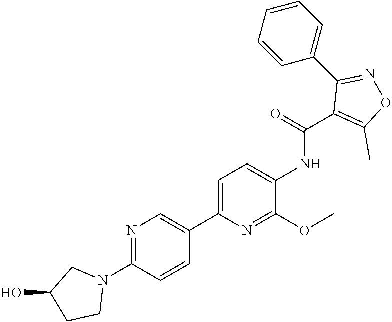 [SMILES: COc1nc(-c2ccc(N3CC[C@@H](O)C3)nc2)ccc1NC(=O)c1c(-c2ccccc2)noc1C]

By way of method described for Example 41, substituting (2-methoxypyrimidin-5-yl) boronic acid for (3R)-1-[5-(tetramethyl-1,3,2-dioxaborolan-2-yl)pyridin-2-yl] pyrrolidin-3-ol (91 mg, 46% yield). ¹H NMR (500 MHZ, DMSO-d₆) δ9.42 (s, 1H), 8.81 (d, J=2.5 Hz, 1H), 8.22 (d, J=8.1 Hz, 1H), 8.14 (dd, J=8.9, 2.5 Hz, 1H), 7.72 (d, J=6.5 Hz, 2H), 7.64-7.49 (m, 3H), 7.45 (d, J=8.1 Hz, 1H), 6.51 (d, J=8.9 Hz, 1H), 5.07-4.91 (m, 1H), 4.40 (s, 1H), 3.89 (s, 3H), 3.62-3.42 (m, 3H), 3.42-3.33 (m, 1H), 2.67 (s, 3H), 2.11-1.97 (m, 1H), 1.97-1.84 (m, 1H). m/z 472.2 [M+H]⁺.

### Example 100 N-(2-Methoxy-6-(1-methyl-1H-pyrazolo[4,3-b]pyridin-6-yl)pyridin-3-yl)-5-methyl-3-phenylisoxazole-4-carboxamide

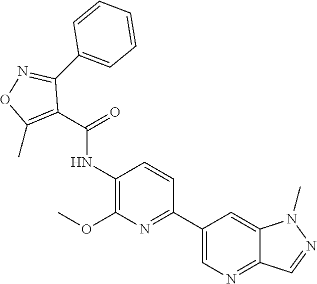 [SMILES: COc1nc(-c2cnc3cnn(C)c3c2)ccc1NC(=O)c1c(-c2ccccc2)noc1C]

By way of method described for Example 41, substituting (2-methoxypyrimidin-5-yl) boronic acid with (1-methyl-1H-pyrazolo[4,3-b]pyridin-6-yl) boronic acid (41 mg, 20% yield). ¹H NMR (500 MHZ, DMSO-d₆) δ9.51 (s, 1H), 9.27 (s, 1H), 8.72 (d, J=1.8 Hz, 1H), 8.45 (s, 1H), 8.30 (s, 1H), 7.83 (d, J=8.1 Hz, 1H), 7.73 (d, J=7.1 Hz, 2H), 7.62-7.52 (m, 3H), 4.16 (s, 3H), 3.97 (s, 3H), 2.68 (s, 3H). m/z 441.2 [M+H]⁺.

### Example 101 N-(6-(1,5-Dimethyl-1H-1,2,3-triazol-4-yl)-2-methoxypyridin-3-yl)-5-methyl-3-phenylisoxazole-4-carboxamide

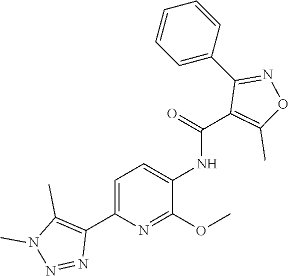 [SMILES: COc1nc(-c2nnn(C)c2C)ccc1NC(=O)c1c(-c2ccccc2)noc1C]

By way of method described for Example 41, substituting (2-methoxypyrimidin-5-yl) boronic acid with 1,5-dimethyl-4-(tetramethyl-1,3,2-dioxaborolan-2-yl)-1H-1,2,3-triazole (38 mg, 18% yield). ¹H NMR (500 MHZ, DMSO-d₆) δ9.49 (s, 1H), 8.32 (d, J=8.1 Hz, 1H), 7.75-7.64 (m, 3H), 7.60-7.51 (m, 3H), 3.98 (s, 3H), 3.88 (s, 3H), 2.68 (s, 3H), 2.67 (s, 3H). m/z 405.1 [M+H]⁺.

### Example 102 N-(2-Methoxy-6-(1-methyl-1H-1,2,3-triazol-5-yl)pyridin-3-yl)-5-methyl-3-phenyl-isoxazole-4-carboxamide

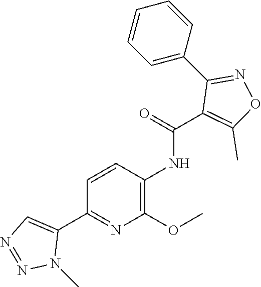 [SMILES: COc1nc(-c2cnnn2C)ccc1NC(=O)c1c(-c2ccccc2)noc1C]

By way of method described for Example 41, substituting (2-methoxypyrimidin-5-yl) boronic acid with 1-methyl-5-(tetramethyl-1,3,2-dioxaborolan-2-yl)-1H-1,2,3-triazole (33 mg, 16% yield). ¹H NMR (500 MHZ, DMSO-d₆) δ9.56 (s, 1H), 8.42 (br s, 1H), 8.24 (s, 1H), 7.70 (d, J=7.0 Hz, 2H), 7.61-7.50 (m, 4H), 4.35 (s, 3H), 3.89 (s, 3H), 2.66 (s, 3H). m/z 391.2 [M+H]⁺.

### Example 103 N-(6-Methoxy-2′-(methylamino)-[2,4′-bipyridin]-5-yl)-5-methyl-3-phenylisoxazole-4-carboxamide

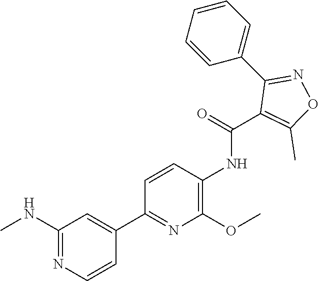 [SMILES: CNc1cc(-c2ccc(NC(=O)c3c(-c4ccccc4)noc3C)c(OC)n2)ccn1]

By way of method described for Example 41, substituting (2-methoxypyrimidin-5-yl) boronic acid for N-methyl-4-(tetramethyl-1,3,2-dioxaborolan-2-yl)pyridin-2-amine (34 mg, 17% yield). ¹H NMR (500 MHZ, DMSO-d₆) δ9.48 (s, 1H), 8.41 (s, 1H), 8.05 (d, J=5.4 Hz, 1H), 7.71 (d, J=7.5 Hz, 2H), 7.63-7.51 (m, 4H), 7.15-7.09 (m, 2H), 6.70 (s, 1H), 3.90 (s, 3H), 2.83 (s, 3H), 2.66 (s, 3H). m/z 416.2 [M+H]⁺.

### Example 104 N-(2-Methoxy-6-(pyridazin-4-yl)pyridin-3-yl)-5-methyl-3-phenylisoxazole-4-carboxamide

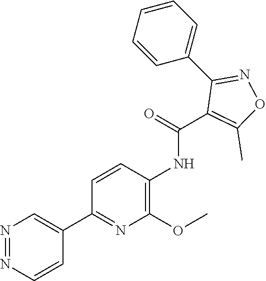 [SMILES: COc1nc(-c2ccnnc2)ccc1NC(=O)c1c(-c2ccccc2)noc1C]

By way of method described for Example 41, substituting (2-methoxypyrimidin-5-yl) boronic acid with 4-(tetramethyl-1,3,2-dioxaborolan-2-yl)pyridazine (59 mg, 29% yield). ¹H NMR (500 MHZ, DMSO-d₆) δ9.89 (s, 1H), 9.54 (s, 1H), 9.32 (d, J=5.6 Hz, 1H), 8.51 (s, 1H), 8.24 (dd, J=5.5, 2.4 Hz, 1H), 7.93 (d, J=8.1 Hz, 1H), 7.70 (dd, J=7.2, 1.4 Hz, 2H), 7.62-7.51 (m, 3H), 3.94 (s, 3H), 2.67 (s, 3H). m/z 388.2 [M+H]⁺.

### Example 105 N-(2-Methoxy-6-(1H-pyrrolo[2,3-b]pyridin-3-yl)pyridin-3-yl)-5-methyl-3-phenylisoxazole-4-carboxamide

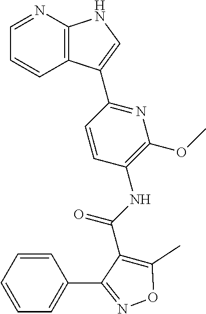 [SMILES: COc1nc(-c2c[nH]c3ncccc23)ccc1NC(=O)c1c(-c2ccccc2)noc1C]

By way of method described for Example 41, substituting (2-methoxypyrimidin-5-yl) boronic acid with 3-(tetramethyl-1,3,2-dioxaborolan-2-yl)-1H-pyrrolo[2,3-b]pyridine (50 mg, 25% yield). ¹H NMR (500 MHZ, DMSO-d₆) δ12.02 (s, 1H), 9.46 (s, 1H), 8.76 (d, J=7.9 Hz, 1H), 8.27 (d, J=4.6 Hz, 1H), 8.24-8.17 (m, 2H), 7.77-7.71 (m, 2H), 7.58-7.49 (m, 4H), 7.19 (dd, J=8.0, 4.7 Hz, 1H), 3.98 (s, 3H), 2.67 (s, 3H). m/z 426.2 [M+H]⁺.

### Example 106 N-(6-([1,3] Dioxolo[4,5-b]pyridin-6-yl)-2-methoxypyridin-3-yl)-5-methyl-3-phenylisoxazole-4-carboxamide

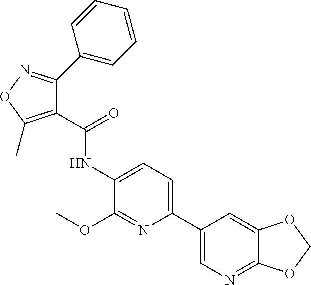 [SMILES: COc1nc(-c2cnc3c(c2)OCO3)ccc1NC(=O)c1c(-c2ccccc2)noc1C]

By way of method described for Example 41, substituting (2-methoxypyrimidin-5-yl) boronic acid for [1,3] dioxolo[4,5-b]pyridin-6-ylboronic acid (84 mg, 43% yield). ¹H NMR (500 MHZ, DMSO-d₆) δ9.47 (s, 1H), 8.36-8.30 (m, 2H), 7.86 (s, 1H), 7.71 (d, J=7.0 Hz, 2H), 7.60-7.51 (m, 4H), 6.21 (s, 2H), 3.90 (s, 3H), 2.66 (s, 3H). m/z 431.1 [M+H]⁺.

### Example 107 N-(6′-Hydroxy-6-methoxy-5′-(trifluoromethyl)-[2,3′-bipyridin]-5-yl)-5-methyl-3-phenylisoxazole-4-carboxamide

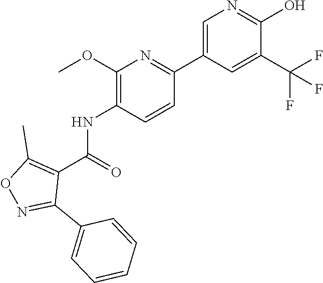 [SMILES: COc1nc(-c2cnc(O)c(C(F)(F)F)c2)ccc1NC(=O)c1c(-c2ccccc2)noc1C]

By way of method described for Example 41, substituting (2-methoxypyrimidin-5-yl) boronic acid with (6-hydroxy-5-(trifluoromethyl)pyridin-3-yl) boronic acid (215 mg, 86% yield). ¹H NMR (500 MHz, DMSO-d₆) δ12.71 (s, 1H), 9.47 (s, 1H), 8.55 (s, 1H), 8.39 (s, 1H), 8.31-8.26 (m, 1H), 7.73-7.68 (m, 2H), 7.58-7.52 (m, 4H), 3.87 (s, 3H), 2.65 (s, 3H). m/z 471.1 [M+H]⁺.

### Example 108 N-(6′-Acetamido-6-methoxy-[2,3′-bipyridin]-5-yl)-5-methyl-3-phenylisoxazole-4-carboxamide

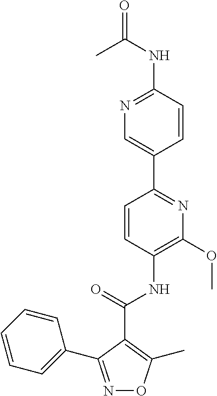 [SMILES: COc1nc(-c2ccc(NC(C)=O)nc2)ccc1NC(=O)c1c(-c2ccccc2)noc1C]

By way of method described for Example 41, substituting (2-methoxypyrimidin-5-yl) boronic acid with (6-acetamidopyridin-3-yl) boronic acid (179 mg, 72% yield). ¹H NMR (600 MHZ, Chloroform-d) δ8.84 (s, 1H), 8.71 (d, J=8.3 Hz, 1H), 8.26-8.22 (m, 2H), 8.02 (s, 1H), 7.76 (s, 1H), 7-70-7.60 (m, 3H), 7.58 (d, J=7.5 Hz, 2H), 7.30 (d, J=8.3 Hz, 1H), 3.70 (s, 3H), 2.83 (s, 3H), 2.23 (s, 3H). m/z 444.0 [M+H]⁺.

### Example 109 N-(2-Methoxy-6-(5-methoxy-1-methyl-1H-pyrrolo[2,3-c]pyridin-3-yl)pyridin-3-yl)-5-methyl-3-phenylisoxazole-4-carboxamide

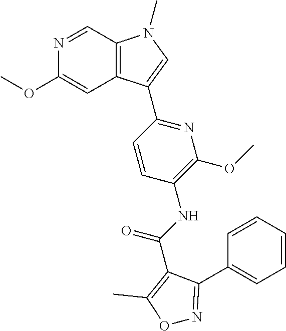 [SMILES: COc1cc2c(-c3ccc(NC(=O)c4c(-c5ccccc5)noc4C)c(OC)n3)cn(C)c2cn1]

By way of method described for Example 41, substituting (2-methoxypyrimidin-5-yl) boronic acid for 5-methoxy-1-methyl-3-(tetramethyl-1,3,2-dioxaborolan-2-yl)-1H-pyrrolo[2,3-c] pyridine (65 mg, 32% yield). ¹H NMR (500 MHZ, DMSO-d₆) δ9.46 (s, 1H), 8.49 (s, 1H), 8.25 (s, 1H), 8.19 (d, J=8.2 Hz, 1H), 7.76-7.71 (m, 2H), 7.66 (s, 1H), 7.58-7.53 (m, 3H), 7.39 (d, J=8.1 Hz, 1H), 3.97 (s, 3H), 3.90 (s, 3H), 3.87 (s, 3H), 2.66 (s, 3H). m/z 470.2 [M+H]⁺.

### Example 110 N-(2-Methoxy-6-(1-(pyridin-4-yl)-1H-pyrazol-4-yl)pyridin-3-yl)-5-methyl-3-phenylisoxazole-4-carboxamide

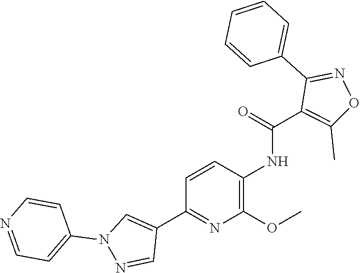 [SMILES: COc1nc(-c2cnn(-c3ccncc3)c2)ccc1NC(=O)c1c(-c2ccccc2)noc1C]

By way of method described for Example 41, substituting (2-methoxypyrimidin-5-yl) boronic acid with 4-[4-(tetramethyl-1,3,2-dioxaborolan-2-yl)-1H-pyrazol-1-yl] pyridine (47 mg, 23% yield). ¹H NMR (500 MHZ, DMSO-d₆) δ9.45 (s, 1H), 9.23 (s, 1H), 8.71-8.66 (m, 2H), 8.40 (s, 1H), 8.30 (d, J=8.1 Hz, 1H), 7.97-7.92 (m, 2H), 7.72 (d, J=6.8 Hz, 2H), 7.56 (d, J=7.1 Hz, 3H), 7.44 (d, J=8.0 Hz, 1H), 3.93 (s, 3H), 2.67 (s, 3H). m/z 453.1 [M+H]⁺.

### Example 111 N-(6-(1,2-Dimethyl-1H-benzo[d]imidazol-5-yl)-2-methoxypyridin-3-yl)-5-methyl-3-phenylisoxazole-4-carboxamide

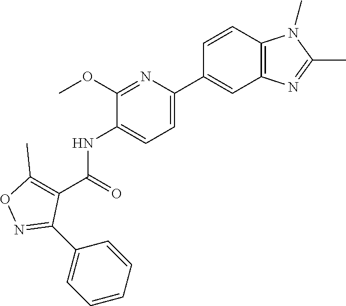 [SMILES: COc1nc(-c2ccc3c(c2)nc(C)n3C)ccc1NC(=O)c1c(-c2ccccc2)noc1C]

By way of method described for Example 41, substituting (2-methoxypyrimidin-5-yl) boronic acid with 1,2-dimethyl-5-(tetramethyl-1,3,2-dioxaborolan-2-yl)-1H-1,3-benzodiazole (32 mg, 16% yield). ¹H NMR (500 MHZ, DMSO-d₆) δ9.45 (s, 1H), 8.30 (d, J=8.2 Hz, 1H), 8.23 (s, 1H), 7.95 (d, J=8.4 Hz, 1H), 7.75-7.70 (m, 2H), 7.62 (d, J=8.1 Hz, 1H), 7.60-7.51 (m, 4H), 3.94 (s, 3H), 3.76 (s, 3H), 2.67 (s, 3H), 2.54 (s, 3H). m/z 454.1 [M+H]⁺.

### Example 112 N-(2-Methoxy-6-(pyrazolo[1,5-a]pyrimidin-3-yl)pyridin-3-yl)-5-methyl-3-phenylisoxazole-4-carboxamide

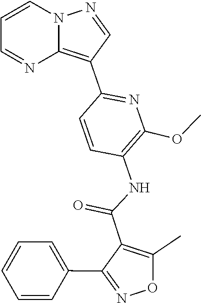 [SMILES: COc1nc(-c2cnn3cccnc23)ccc1NC(=O)c1c(-c2ccccc2)noc1C]

By way of method described for Example 41, substituting (2-methoxypyrimidin-5-yl) boronic acid with 3-(tetramethyl-1,3,2-dioxaborolan-2-yl) pyrazolo[1,5-a]pyrimidine (190 mg, 76% yield). ¹H NMR (500 MHZ, DMSO-d₆) δ9.43 (s, 1H), 9.22 (d, J=7.0 Hz, 1H), 8.80 (s, 1H), 8.78-8.72 (m, 1H), 8.33-8.28 (m, 1H), 8.02 (d, J=8.1 Hz, 1H), 7.73 (d, J=6.7 Hz, 2H), 7.58-7.53 (m, 3H), 7.20-7.14 (m, 1H), 3.93 (s, 3H), 2.67 (s, 3H). m/z 427.1 [M+H]⁺.

### Example 113 N-(2-Methoxy-6-(pyrazin-2-yl)pyridin-3-yl)-5-methyl-3-phenylisoxazole-4-carboxamide

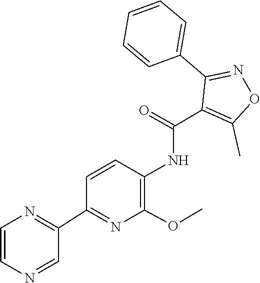 [SMILES: COc1nc(-c2cnccn2)ccc1NC(=O)c1c(-c2ccccc2)noc1C]

By way of method described for Example 41, substituting (2-methoxypyrimidin-5-yl) boronic acid with pyrazin-2-ylboronic acid (70 mg, 28% yield). ¹H NMR (500 MHZ, DMSO-d₆) δ9.51 (s, 1H), 9.51-9.48 (m, 1H), 8.85-8.79 (m, 1H), 8.73-8.69 (m, 1H), 8.67 (d, J=2.6 Hz, 1H), 7.99 (d, J=8.1 Hz, 1H), 7.74-7.69 (m, 2H), 7.62-7.52 (m, 3H), 3.95 (s, 3H), 2.68 (s, 3H). m/z 388.2 [M+H]⁺.

### Example 114 N-(2-Methoxy-6-(2-oxo-2,3-dihydro-1H-pyrrolo[2,3-b]pyridin-5-yl)pyridin-3-yl)-5-methyl-3-phenylisoxazole-4-carboxamide

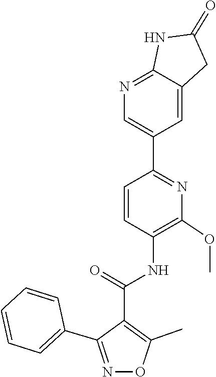 [SMILES: COc1nc(-c2cnc3c(c2)CC(=O)N3)ccc1NC(=O)c1c(-c2ccccc2)noc1C]

By way of method described for Example 41, substituting (2-oxo-2,3-dihydro-1H-pyrrolo[2,3-b]pyridin-5-yl) boronic acid for (2-methoxypyrimidin-5-yl) boronic acid (210 mg, 86% yield). ¹H NMR (600 MHZ, Chloroform-d) δ8.75-8.67 (m, 2H), 8.12-8.01 (m, 2H), 7.75 (s, 1H), 7.70-7.50 (m, 5H), 3.70 (s, 3H), 3.63 (s, 2H), 2.84 (s, 3H). m/z 483.0 [M+CH₃CN+H]⁺.

### Example 115 N-(2-Methoxy-6-(3-methyl-1-(thiazol-2-yl)-1H-pyrazol-4-yl)pyridin-3-yl)-5-methyl-3-phenylisoxazole-4-carboxamide

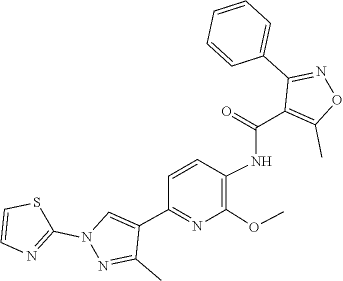 [SMILES: COc1nc(-c2cn(-c3nccs3)nc2C)ccc1NC(=O)c1c(-c2ccccc2)noc1C]

By way of method described for Example 41, substituting (2-methoxypyrimidin-5-yl) boronic acid with 2-[3-methyl-4-(tetramethyl-1,3,2-dioxaborolan-2-yl)-1H-pyrazol-1-yl]-1,3-thiazole (200 mg, 79% yield). ¹H NMR (500 MHZ, DMSO-d₆) δ9.48 (s, 1H), 8.96 (s, 1H), 8.27 (d, J=8.0 Hz, 1H), 7.75-7.70 (m, 2H), 7.65 (d, J=3.4 Hz, 1H), 7.59-7.43 (m, 5H), 3.89 (s, 3H), 2.66 (s, 3H), 2.61 (s, 3H). m/z 473.2 [M+H]⁺.

### Example 116 N-(5′-Fluoro-6-methoxy-6′-oxo-1′,6′-dihydro-[2,3′-bipyridin]-5-yl)-5-methyl-3-phenylisoxazole-4-carboxamide

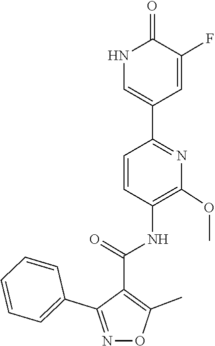 [SMILES: COc1nc(-c2c[nH]c(=O)c(F)c2)ccc1NC(=O)c1c(-c2ccccc2)noc1C]

By way of method described for Example 41, substituting (2-methoxypyrimidin-5-yl) boronic acid with (5-fluoro-6-oxo-1,6-dihydropyridin-3-yl) boronic acid (186 mg, 74% yield). ¹H NMR (600 MHZ, Chloroform-d) δ8.70 (s, 1H), 7.90-7.77 (m, 2H), 7.73 (s, 1H), 7.70-7.50 (m, 6H), 7.05 (d, J=8.4 Hz, 1H), 3.66 (s, 3H), 2.83 (s, 3H). m/z 462.0 [M+CH₃CN+H]⁺.

### Example 117 N-(2-Methoxy-6-(1-oxo-1,2-dihydrophthalazin-6-yl)pyridin-3-yl)-5-methyl-3-phenylisoxazole-4-carboxamide

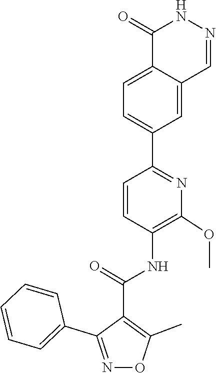 [SMILES: COc1nc(-c2ccc3c(=O)[nH]ncc3c2)ccc1NC(=O)c1c(-c2ccccc2)noc1C]

By way of method described for Example 41, substituting (2-methoxypyrimidin-5-yl) boronic acid with (1-oxo-1,2-dihydrophthalazin-6-yl) boronic acid (160 mg, 65% yield). ¹H NMR (600 MHz, Chloroform-d) δ9.77 (s, 1H), 8.80 (d, J=8.3 Hz, 1H), 8.44 (d, J=8.5 Hz, 1H), 8.37 (d, J=8.4 Hz, 1H), 8.28 (s, 1H), 8.21 (s, 1H), 7.83 (s, 1H), 7.69-7.61 (m, 3H), 7.61-7.55 (m, 2H), 7.51 (d, J=8.1 Hz, 1H), 3.76 (s, 3H), 2.85 (s, 3H). m/z 495.0 [M+CH₃CN+H]⁺.

### Example 118 N-(2-Methoxy-6-(1H-pyrrolo[2,3-b]pyridin-5-yl)pyridin-3-yl)-5-methyl-3-phenylisoxazole-4-carboxamide

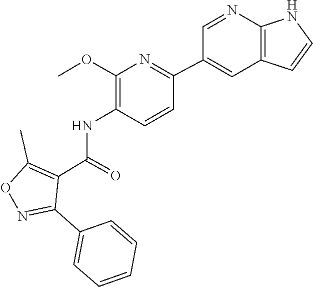 [SMILES: COc1nc(-c2cnc3[nH]ccc3c2)ccc1NC(=O)c1c(-c2ccccc2)noc1C]

By way of method described for Example 41, substituting (2-methoxypyrimidin-5-yl) boronic acid with 5-(tetramethyl-1,3,2-dioxaborolan-2-yl)-1H-pyrrolo[2,3-b]pyridine (190 mg, 75% yield). ¹H NMR (500 MHZ, DMSO-d₆) δ11.77 (s, 1H), 9.48 (s, 1H), 8.95 (s, 1H), 8.59 (s, 1H), 8.35-8.31 (m, 1H), 7.75-7.71 (m, 2H), 7.65 (d, J=8.1 Hz, 1H), 7.58-7.53 (m, 3H), 7.53-7.49 (m, 1H), 6.53 (s, 1H), 3.95 (s, 3H), 2.67 (s, 3H). m/z 426.2 [M+H]⁺.

### Example 119 N-(2-Methoxy-6-(2-oxo-1,4-dihydro-2H-benzo[d] [1,3]oxazin-6-yl)pyridin-3-yl)-5-methyl-3-phenylisoxazole-4-carboxamide

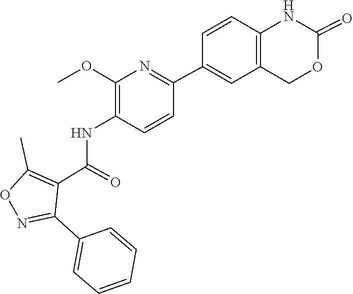 [SMILES: COc1nc(-c2ccc3c(c2)COC(=O)N3)ccc1NC(=O)c1c(-c2ccccc2)noc1C]

By way of method described for Example 41, substituting (2-methoxypyrimidin-5-yl) boronic acid with 6-(tetramethyl-1,3,2-dioxaborolan-2-yl)-2,4-dihydro-1H-3,1-benzoxazin-2-one (193 mg, 78% yield). ¹H NMR (500 MHZ, DMSO-d₆) δ10.30 (s, 1H), 9.45 (s, 1H), 8.31 (s, 1H), 7.97 (d, J=8.4 Hz, 1H), 7.93 (s, 1H), 7.74-7.69 (m, 2H), 7.59-7.48 (m, 4H), 6.96 (d, J=8.3 Hz, 1H), 5.37 (s, 2H), 3.90 (s, 3H), 2.66 (s, 3H). m/z 457.1 [M+H]⁺.

### Example 120 N-(5-Isoxazol-4-yl-3-methoxy-pyrazin-2-yl)-5-methyl-3-phenyl-isoxazole-4-carboxamide

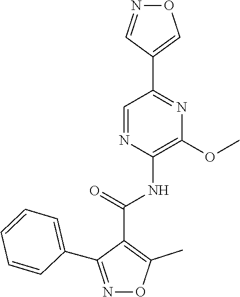 [SMILES: COc1nc(-c2cnoc2)cnc1NC(=O)c1c(-c2ccccc2)noc1C]

A 5 mL microwave vial charged with Intermediate 6 (100 mg, 0.26 mmol), isoxazole-4-boronic acid (35 mg, 0.31 mmol), KOAc (101 mg, 1.03 mmol), Pd-118 (17 mg, 0.03 mmol), 1,4-dioxane (2 mL) and water (0.2 mL). The vial was sealed, purged with nitrogen and degassed before heated at 80° C. for 1 h. The reaction mixture was concentrated under reduced pressure and the residue was redissolved in DCM (25 mL) and washed with water (25 mL). The organic phase was concentrated under reduced pressure. The residue was adsorbed onto silica and purified by flash column chromatography (4 g silica, elution with a 0-90% EtOAc/PE gradient) to afford N-(5-isoxazol-4-yl-3-methoxy-pyrazin-2-yl)-5-methyl-3-phenyl-isoxazole-4-carboxamide (23 mg, 22% yield) as a tan solid. ¹H NMR (600 MHZ, Chloroform-d) δ8.84 (s, 1H), 8.66 (s, 1H), 8.13 (s, 1H), 7.93 (s, 1H), 7.66-7.59 (m, 3H), 7.57 (t, J=7.6 Hz, 2H), 3.72 (s, 3H), 2.84 (s, 3H). m/z 378.0 [M+H]⁺.

### Example 121 N-[6-(1H-Imidazol-2-yl)-2-methoxy-3-pyridyl]-5-methyl-3-phenyl-isoxazole-4-carboxamide

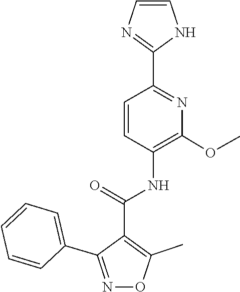 [SMILES: COc1nc(-c2ncc[nH]2)ccc1NC(=O)c1c(-c2ccccc2)noc1C]

A microwave vial was charged with Intermediate 7 (50 mg, 0.13 mmol), 2-bromo-1H-imidazole (19 mg, 0.13 mmol), 1,4-dioxane (1 mL) and water (0.5 mL). The reaction mixture was evacuated and backfilled with nitrogen three times. Then, Na₂CO₃ (68 mg, 0.64 mmol) and tetrakis(triphenylphosphine) palladium (0) (29 mg, 0.03 mmol) were added. The reaction mixture was then evacuated and backfilled with nitrogen three times before being irradiated under microwave at 120° C. for 1 h. The reaction mixture was concentrated to dryness. The residue was taken up in DCM (10 mL) and the organics were washed with water (10 mL) and brine (10 mL). The collected organics were dried over MgSO₄ and concentrated to dryness under reduced pressure. Purification by flash silica column chromatography on an ISCO system (elution with 4% MeOH/DCM gradient) yielded N-[6-(1H-imidazol-2-yl)-2-methoxy-3-pyridyl]-5-methyl-3-phenyl-isoxazole-4-carboxamide (10.5 mg, 21% yield) as a colourless solid. ¹H NMR (600 MHZ, Chloroform-d) δ9.89 (s, 1H), 8.76 (d, J=8.1 Hz, 1H), 7.78-7.69 (m, 2H), 7.68-7.53 (m, 5H), 7.13 (s, 2H), 3.66 (s, 3H), 2.83 (s, 3H). m/z 375.9 [M+H]⁺.

### Example 122 N-[2-Methoxy-6-(2H-tetrazol-5-yl)-3-pyridyl]-5-methyl-3-phenyl-isoxazole-4-carboxamide

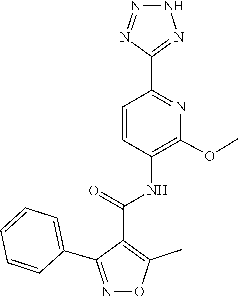 [SMILES: COc1nc(-c2nn[nH]n2)ccc1NC(=O)c1c(-c2ccccc2)noc1C]

### Step 1

In a microwave vial was charged with Intermediate 1 (500 mg, 1.29 mmol) and DMSO (10 mL). To this was added copper(I) cyanide (90 mg, 1.29 mmol), the vial was sealed and heated at 100° C. o/n. The reaction mixture was diluted with EtOAc (15 mL) and was washed with water (3×3 mL). The organics were separated, dried over MgSO₄ and evaporated. The crude N-(6-cyano-2-methoxy-3-pyridyl)-5-methyl-3-phenyl-isoxazole-4-carboxamide thus obtained as an off-white solid (approx. 50 mg) was used directly in the next step. ¹H NMR (600 MHZ, Chloroform-d) δ8.75 (d, J=8.0 Hz, 1H), 7.85 (s, 1H), 7.68-7.55 (m, 5H), 7.31 (d, J=8.1 Hz, 1H), 3.62 (s, 3H), 2.83 (s, 3H).

### Step 2

To solution of N-(6-cyano-2-methoxy-3-pyridyl)-5-methyl-3-phenyl-isoxazole-4-carboxamide (50 mg, 0.15 mmol) in DMF (1 mL) were added sodium azide (29 mg, 0.45 mmol) and NH₄Cl (80 mg, 1.49 mmol). The reaction mixture was heated at 120° C. for 3 days. Then, the reaction mixture was mixed with saturated NaHCO₃ (10 mL) followed by extraction with EtOAc (3×5 mL). The aqueous phase was carefully acidified by drop-wise addition of concentrated HCl to pH=2 followed by extraction with DCM (3×5 mL). The combined organics were washed with water (3 mL), dried over MgSO₄ and evaporated. The crude product obtained was triturated with MeCN to give N-[2-methoxy-6-(2H-tetrazol-5-yl)-3-pyridyl]-5-methyl-3-phenyl-isoxazole-4-carboxamide (18 mg, 30% yield) as a colourless solid. ¹H NMR (600 MHZ, DMSO-d₆) δ9.52 (s, 1H), 8.54 (s, 1H), 7.82 (d, J=8.0 Hz, 1H), 7.68 (d, J=7.2 Hz, 2H), 7.60-7.49 (m, 3H), 3.93 (s, 3H), 2.65 (s, 3H). m/z 378.0 [M+H]⁺.

### Example 123 N-(2-Methoxy-6-oxazol-2-yl-3-pyridyl)-5-methyl-3-phenyl-isoxazole-4-carboxamide

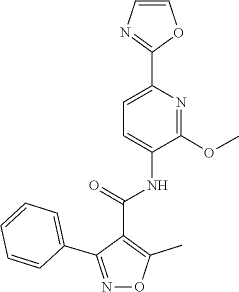 [SMILES: COc1nc(-c2ncco2)ccc1NC(=O)c1c(-c2ccccc2)noc1C]

In a 5 mL microwave vial, a solution of Intermediate 1 (100 mg, 0.26 mmol) and tetrakis(triphenylphosphine) palladium (0) (30 mg, 0.03 mmol) in 1,4-dioxane (2.5 mL) was treated with 2-(tributylstannyl)-1,3-oxazole (0.12 mL, 0.36 mmol). The vial was sealed, purged with nitrogen and degassed before heated at 120° C. for 24 h, then at room temperature for 2 days. The reaction mixture was partitioned between EtOAc (25 mL) and water (25 mL) and the layers were separated. The organic layer was washed with brine, dried over MgSO₄, filtered and concentrated under reduced pressure. The residue was adsorbed onto silica and purified by flash column chromatography (4 g silica, elution with a 0-90% EtOAc/PE gradient) to afford a solid which was not sufficiently pure. The material was purified again by flash column chromatography (4 g silica, elution with a 0-5% MeOH/DCM gradient) to afford N-(2-methoxy-6-oxazol-2-yl-3-pyridyl)-5-methyl-3-phenyl-isoxazole-4-carboxamide (24 mg, 23% yield) as a colourless solid. ¹H NMR (600 MHZ, Chloroform-d) δ8.78 (d, J=8.2 Hz, 1H), 7.84 (s, 1H), 7.72 (s, 1H), 7.70 (d, J=8.2 Hz, 1H), 7.63 (d, J=7.3 Hz, 3H), 7.58 (t, J=7.6 Hz, 2H), 5.30 (s, 1H), 3.73 (s, 3H), 2.84 (s, 3H). m/z 376.9 [M+H]⁺.

### Example 124 N-(6-(Isoxazol-4-yl)-2-methoxypyridin-3-yl)-4-methyl-1-phenyl-1H-1,2,3-triazole-5-carboxamide

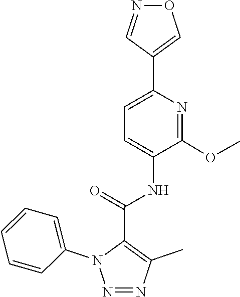 [SMILES: COc1nc(-c2cnoc2)ccc1NC(=O)c1c(C)nnn1-c1ccccc1]

### Step 1—Synthesis of Intermediate 10-N-(6-Bromo-2-methoxypyridin-3-yl)-4-methyl-1-phenyl-1H-1,2,3-triazole-5-carboxamide

4-Methyl-1-phenyl-1H-1,2,3-triazole-5-carboxylic acid (0.95 g, 4.68 mmol) was dissolved in SOCl₂ (3.75 mL, 51.4 mmol) and the reaction mixture was heated to 65° C. overnight. After cooling to rt, excess SOCl₂ was removed under reduced pressure in a fumehood. The residue was dissolved in DCM (10 mL) and then added drop-wise to a solution of 6-bromo-2-methoxy-3-pyridinamine (0.95 g, 4.68 mmol) and Et₃N (0.78 mL, 5.61 mmol) in DCM (10 mL) at 0° C. The reaction mixture was stirred at 0° C. for 2 h, allowing to warm to rt. The reaction mixture was diluted with EtOAc (20 mL) and washed sequentially with water (20 mL), 1 M NaOH (2×20 mL), 1 M HCl (3×20 mL) and brine (20 mL). The organic layer was dried over MgSO₄, filtered and concentrated under reduced pressure to afford N-(6-bromo-2-methoxypyridin-3-yl)-4-methyl-1-phenyl-1H-1,2,3-triazole-5-carboxamide (1.20 g, 59% yield) as a brown solid. ¹H NMR (500 MHZ, Chloroform-d) δ8.48 (d, J=8.2 Hz, 1H), 7.72 (br s, 1H), 7.56-7.63 (m, 3H), 7.51-7.56 (m, 2H), 7.06 (d, J=8.2 Hz, 1H), 3.85 (s, 3H), 2.69 (s, 3H). m/z 388.0 [M+H, ⁷⁹Br]⁺, 390.0 [M+H, ⁸¹Br]⁺.

### Step 2

A 20 mL microwave vial was charged with Intermediate 10 (200 mg, 0.52 mmol), isoxazole-4-boronic acid (64 mg, 0.57 mmol), KF (90 mg, 1.55 mmol), 1,4-dioxane (9 mL) and water (3 mL). The reaction mixture was degassed with nitrogen for 5 mins. Pd-118 (34 mg, 0.05 mmol) was quickly added and the mixture further degassed with nitrogen before stirring at 35° C. o/n. The reaction mixture was then concentrated under reduced pressure. The dark residue dissolved in DCM (20 mL) and washed with water (2×10 mL). The organic layer was dried over MgSO₄, filtered and concentrated under reduced pressure. The residue was purified by flash column chromatography (12 g, silica, 0-100% EtOAc/PE over 25 CV's). CV's containing product were combined and evaporated under reduced pressure. The precipitate was then dried under vacuum at 40° C. to afford N-(6-(isoxazol-4-yl)-2-methoxypyridin-3-yl)-4-methyl-1-phenyl-1H-1,2,3-triazole-5-carboxamide (94 mg, 46% yield) as a yellow solid. ¹H NMR (400 MHZ, Chloroform-d) δ8.83 (s, 1H), 8.68 (s, 1H), 8.63 (d, J=8.1 Hz, 1H), 7.88 (br s, 1H), 7.49-7.69 (m, 5H), 7.07 (d, J=8.2 Hz, 1H), 3.90 (s, 3H), 2.71 (s, 3H). m/z 377.1 [M+H]⁺.

### Example 125 N-[2-Methoxy-6-[2-(methoxymethyl)pyrimidin-5-yl]-3-pyridyl]-5-methyl-3-phenyl-isoxazole-4-carboxamide

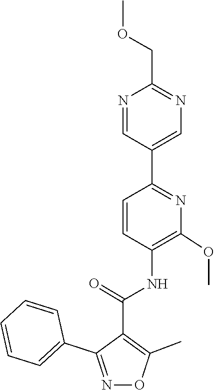 [SMILES: COCc1ncc(-c2ccc(NC(=O)c3c(-c4ccccc4)noc3C)c(OC)n2)cn1]

A solution of Intermediate 7 (80 mg, 0.18 mmol) and KF (32 mg, 0.55 mmol) in 1,4-dioxane (3.2 mL) and water (0.8 mL) was treated with a 5-bromo-2-(methoxymethyl)pyrimidine (45 mg, 0.22 mmol) and the mixture sparged with nitrogen for 10 mins. The reaction was then charged with Pd-118 (12 mg, 0.02 mmol), sealed and heated at 40° C. overnight. The reaction mixture was concentrated under reduced pressure and taken up in chloroform. Water was added and organics separated using a phase separator. The organics were reduced under reduced pressure and the residue purified by flash silica column chromatography on an ISCO system to give N-[2-methoxy-6-[2-(methoxymethyl)pyrimidin-5-yl]-3-pyridyl]-5-methyl-3-phenyl-isoxazole-4-carboxamide (58 mg, 69% yield). ¹H NMR (600 MHz, Chloroform-d) δ9.24 (s, 2H), 8.77 (d, J=8.1 Hz, 1H), 7.79 (s, 1H), 7.66-7.61 (m, 3H), 7.62-7.55 (m, 2H), 7.36 (d, J=8.1 Hz, 1H), 4.74 (s, 2H), 3.70 (s, 3H), 3.57 (s, 3H), 2.84 (s, 3H). m/z 432.0 [M+H]⁺.

### Example 126 N-[6-[2-(Aminomethyl)pyrimidin-5-yl]-2-methoxy-3-pyridyl]-5-methyl-3-phenyl-isoxazole-4-carboxamide hydrochloride

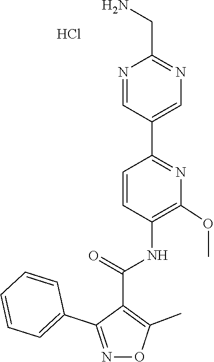 [SMILES: COc1nc(-c2cnc(CN)nc2)ccc1NC(=O)c1c(-c2ccccc2)noc1C ; Cl]

### Step 1—Synthesis of Intermediate 11-tert-Butyl N-[(5-bromopyrimidin-2-yl)methyl]carbamate

A 7 M solution of NH₃ in MeOH (10 mL, 70 mmol) was added to Intermediate 8 (800 mg, 3.18 mmol) and the solution stirred at RT. After about 1 h a suspension had formed. After 4 h, the mixture was concentrated to give an off-white solid. DCM (15 mL) was added, followed by Et₃N (0.89 mL, 6.35 mmol). The open flask was stirred for about 20 min. Di-tert-butyl dicarbonate (832 mg, 3.81 mmol) was then added, a precipitate was briefly formed before forming a yellow solution which was stirred at RT o/n. The reaction was concentrated onto MgSO₄ then purified by flash silica column chromatography on an ISCO system (24 g silica, elution with a 0-20% EtOAc/PE gradient) to give tert-butyl N-[(5-bromopyrimidin-2-yl)methyl]carbamate (450 mg, 44% yield). ¹H NMR (500 MHZ, Chloroform-d) δ8.77 (s, 2H), 5.75 (br s, 1H), 4.57 (d, J=5.3 Hz, 2H), 1.48 (s, 9H). m/z 231.9/233.9 [M-C(CH₃)₃+H]⁺.

### Step 2

A mixture of Intermediate 7 (450 mg, 1.03 mmol), Intermediate 11 (300 mg, 1.04 mmol) and ground KF (180 mg, 3.1 mmol) in 1,4-dioxane (5 mL) and water (2.5 mL) was sparged with nitrogen for 10 mins. Pd-118 (67 mg, 0.10 mmol) was added and mixture heated to 45° C. o/n. The reaction mixture was partitioned between DCM (70 mL) and water (25 mL). The organic layer was separated and the aqueous layer further extracted with DCM (25 mL). The organics were combined, dried over MgSO₄, filtered and concentrated to a brown solid. The solid was suspended in isopropanol (20 mL) and cooled in a freezer (−20° C.) for 1.5 h. The precipitate was isolated by filtration and washed with further cold isopropanol (−20° C., 20 mL) to give tert-butyl N-[[5-[6-methoxy-5-[(5-methyl-3-phenyl-isoxazole-4-carbonyl)amino]-2-pyridyl]pyrimidin-2-yl]methyl]carbamate (520 mg, 88% yield). ¹H NMR (500 MHZ, Chloroform-d) δ9.20 (s, 2H), 8.77 (d, J=8.1 Hz, 1H), 7.81-7.77 (m, 1H), 7.68-7.62 (m, 3H), 7.62-7.55 (m, 2H), 7.35 (d, J=8.1 Hz, 1H), 5.70 (br s, 1H), 4.63 (d, J=5.1 Hz, 2H), 3.69 (s, 3H), 2.84 (s, 3H), 1.48 (s, 9H). m/z 517.2 [M+H]⁺.

### Step 3

A suspension of tert-butyl N-[[5-[6-methoxy-5-[(5-methyl-3-phenyl-isoxazole-4-carbonyl)amino]-2-pyridyl]pyrimidin-2-yl]methyl]carbamate (120 mg, 0.21 mmol) in 1,4-dioxane (1 mL) had a 2 M solution of HCl in ether (5 mL, 10 mmol) added and resultant fine suspension stirred o/n at RT. LCMS of the suspension indicated complete reaction. The solid was isolated by filtration to give a hygroscopic solid that was suspended with sonication in isopropanol (20 mL) before evaporation using a Biotage V10. The resultant solid was dried in a vacuum oven for 3 h at 40° C. to give N-[6-[2-(aminomethyl)pyrimidin-5-yl]-2-methoxy-3-pyridyl]-5-methyl-3-phenyl-isoxazole-4-carboxamide hydrochloride (96 mg, 99% yield). ¹H NMR (500 MHZ, DMSO-d₆) δ9.56 (s, 1H), 9.50 (s, 2H), 8.54-8.36 (m, 4H), 7.84 (d, J=8.2 Hz, 1H), 7.72 (d, J=7.1 Hz, 2H), 7.61-7.53 (m, 3H), 4.39 (s, 2H), 3.94 (s, 3H), 2.68 (s, 3H). m/z 417.1 [M+H]⁺.

### Example 127 N-[6-(6-Aminopyridazin-3-yl)-2-methoxy-3-pyridyl]-5-methyl-3-phenyl-isoxazole-4-carboxamide

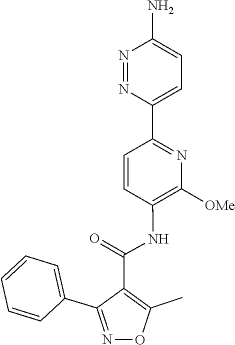 [SMILES: COc1nc(-c2ccc(N)nn2)ccc1NC(=O)c1c(-c2ccccc2)noc1C]

A solution of 6-bromopyridazin-3-amine (40 mg, 0.23 mmol), Intermediate 7 (100 mg, 0.23 mmol) and KF (40 mg, 0.71 mmol) in 1,4-dioxane (4 mL) and water (1 mL) was degassed with nitrogen for 15 min. Pd-118 (15 mg, 0.023 mmol) was added quickly, the solution degassed for a further 10 min before being left to stir at rt overnight. The reaction mixture was diluted with EtOAc (20 mL), washed with water (3×20 mL) and brine (20 mL). The organic extract was dried over MgSO₄, filtered and concentrated under reduced pressure to give the crude product, which was purified by automated column chromatography (gradient over 20 CV from 0-50% EtOAc/n-hexane, followed by a fast gradient to 100% EtOAc) to give the partially purified product, which was triturated with ether (5 mL) with rapid stirring for 15 min. The suspension was left to settle and the supernatant removed by pipetting. This procedure was repeated twice and the resulting solid dried under reduced pressure to give N-[6-(6-aminopyridazin-3-yl)-2-methoxy-3-pyridyl]-5-methyl-3-phenyl-isoxazole-4-carboxamide (11 mg, 12% yield) as a beige solid. ¹H NMR (500 MHZ, Chloroform-d) δ8.77 (d, J=8.2 Hz, 1H), 8.16-8.07 (m, 2H), 7.76 (s, 1H), 7.68-7.60 (m, 3H), 7.57 (t, J=7.4 Hz, 2H), 6.79 (d, J=9.1 Hz, 1H), 4.82 (s, 2H), 3.68 (s, 3H), 2.83 (s, 3H). m/z 403.1 [M+H]⁺.

### Example 128 N-[2-Methoxy-6-(6-methoxypyridazin-3-yl)-3-pyridyl]-5-methyl-3-phenyl-isoxazole-4-carboxamide

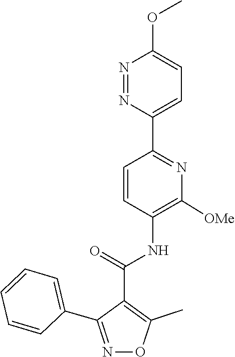 [SMILES: COc1ccc(-c2ccc(NC(=O)c3c(-c4ccccc4)noc3C)c(OC)n2)nn1]

A solution of 3-bromo-6-methoxypyridazine (43 mg, 0.23 mmol), Intermediate 7 (100 mg, 0.23 mmol) and KF (40 mg, 0.69 mmol) in 1,4-dioxane (4 mL) and water (1 mL) was degassed with nitrogen for 15 min. Pd-118 (15 mg, 0.023 mmol) was added quickly, the solution degassed for a further 10 min before being left to stir at rt overnight. The reaction mixture was diluted with EtOAc (20 mL), washed with water (3×20 mL) and brine (20 mL). The organic extract was dried over MgSO₄, filtered and concentrated under reduced pressure to give the crude product, which was purified by automated column chromatography (gradient over 15 CV from 0-50% EtOAc/n-hexane, followed by a fast gradient to 100% EtOAc) to give the partially purified product, which was dissolved in a minimum amount of DCM (around 0.5 mL) and PE (around 3 mL) was added. The solution was left for a few hours for the product to precipitate as the DCM evaporated. The excess PE was removed by pipette and the resulting solid washed three times with PE (the supernatant was removed each time). The solid was dried under reduced pressure to give N-[2-methoxy-6-(6-methoxypyridazin-3-yl)-3-pyridyl]-5-methyl-3-phenyl-isoxazole-4-carboxamide (29 mg, 30% yield). ¹H NMR (500 MHZ, Chloroform-d) δ8.80 (d, J=8.3 Hz, 1H), 8.25 (d, J=9.2 Hz, 1H), 8.17 (d, J=8.3 Hz, 1H), 7.78 (s, 1H), 7.69-7.61 (m, 3H), 7.58 (t, J=7.3 Hz, 2H), 7.01 (d, J=9.2 Hz, 1H), 4.17 (s, 3H), 3.69 (s, 3H), 2.83 (s, 3H). m/z 418.2 [M+H]⁺.

### Example 129 N-[6-(2-Carbamoylpyrimidin-5-yl)-2-methoxy-3-pyridyl]-5-methyl-3-phenyl-isoxazole-4-carboxamide

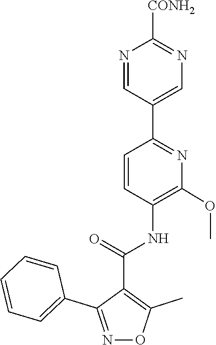 [SMILES: COc1nc(-c2cnc(C(N)=O)nc2)ccc1NC(=O)c1c(-c2ccccc2)noc1C]

A solution of Example 60 (50 mg, 0.12 mmol) and 7 M solution of NH₃ in MeOH (33 μL, 0.23 mmol) in DMF (1 mL) was treated with HATU (66 mg, 0.17 mmol) followed by Hünig's base (0.04 mL, 0.23 mmol) and the reaction mixture stirred at rt overnight. The solvent was removed under reduced pressure and the residue re-dissolved in chloroform. Aqueous NaHCO₃ was added and reaction stirred vigorously before being passed through a hydrophobic frit. The solvent was reduced under reduced pressure to yield a solid which was purified by flash column chromatography using an ISCO system (24 g silica column; 0-10% MeOH/DCM gradient) to give N-[6-(2-carbamoylpyrimidin-5-yl)-2-methoxy-3-pyridyl]-5-methyl-3-phenyl-isoxazole-4-carboxamide (34 mg, 65% yield). ¹H NMR (600 MHz, DMSO-d₆) δ9.51 (s, 2H), 8.46 (s, 1H), 8.21 (s, 1H), 7.92-7.79 (m, 2H), 7.69 (d, J=7.1 Hz, 2H), 7.61-7.43 (m, 3H), 3.92 (s, 3H), 2.66 (s, 3H). One amide NH exchanging. m/z 431.0 [M+H]⁺.

### Example 130 N-[6-[2-(Dimethylcarbamoyl)pyrimidin-5-yl]-2-methoxy-3-pyridyl]-5-methyl-3-phenyl-isoxazole-4-carboxamide

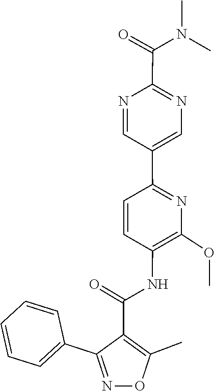 [SMILES: COc1nc(-c2cnc(C(=O)N(C)C)nc2)ccc1NC(=O)c1c(-c2ccccc2)noc1C]

A solution of Example 60 (50 mg, 0.12 mmol) and 2 M dimethylamine (0.12 mL, 0.23 mmol) in DMF (1 mL) was treated with HATU (66.1 mg, 0.1700 mmol) followed by Hünig's base (0.04 mL, 0.23 mmol) and the reaction stirred at rt overnight. The solvent was removed under reduced pressure and the residue re-dissolved in chloroform. Aqueous NaHCO₃ was added and reaction stirred vigorously before being passed through a hydrophobic frit. The solvent was reduced under reduced pressure to yield a solid which was purified by flash column chromatography using an ISCO system (24 g silica column; 0-10% MeOH/DCM gradient) followed by a second column (12 g silica column; 0-100% EtOAc/PE gradient) to give N-[6-[2-(dimethylcarbamoyl)pyrimidin-5-yl]-2-methoxy-3-pyridyl]-5-methyl-3-phenyl-isoxazole-4-carboxamide (28 mg, 50% yield). ¹H NMR (600 MHZ, Chloroform-d) δ9.29 (s, 2H), 8.79 (d, J=8.1 Hz, 1H), 7.81 (s, 1H), 7.67-7.61 (m, 3H), 7.61-7.55 (m, 2H), 7.40 (d, J=8.1 Hz, 1H), 3.70 (s, 3H), 3.16 (s, 3H), 2.98 (s, 3H), 2.83 (s, 3H). m/z 459.0 [M+H]⁺.

### Example 131 N-[6-[2-(Dimethylamino)-[1,2,4]triazolo[1,5-a]pyridin-7-yl]-2-methoxy-3-pyridyl]-5-methyl-3-phenyl-isoxazole-4-carboxamide hydrochloride

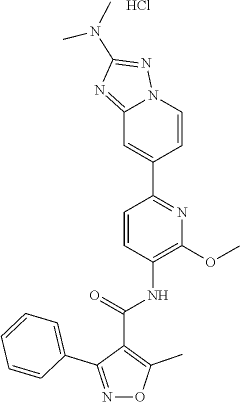 [SMILES: COc1nc(-c2ccn3nc(N(C)C)nc3c2)ccc1NC(=O)c1c(-c2ccccc2)noc1C ; Cl]

A microwave vial was charged with Intermediate 7 (60 mg, 0.14 mmol), 7-bromo-N,N-dimethyl-[1,2,4]triazolo[1,5-a]pyridin-2-amine (42 mg, 0.14 mmol), 1,4-dioxane (1.2 mL) and water (0.3 mL). The reaction mixture was evacuated and backfilled with nitrogen three times. Then, KOAc (40.5 mg, 0.42 mmol) and Pd(dppf)Cl₂ (10 mg, 0.014 mmol) were added. The reaction mixture was then evacuated and backfilled with nitrogen three times before being irradiated under microwave at 120° C. for 1 h. The reaction mixture was concentrated to dryness. The residue was taken up in DCM (10 mL) and the organics were washed with water (10 mL) and brine (10 mL). The collected organics were dried over MgSO₄, filtered and concentrated to dryness under reduced pressure. The residue was purified by flash silica column chromatography on an ISCO system (elution with 3% MeOH/DCM gradient). The desired fractions were concentrated to dryness under reduced pressure. The product thus obtained as an off-white solid was dissolved in DCM (1 mL) followed by addition of HCl in ether (1M solution in ether, 1 mL). Instantaneous formation of solid was noted. The reaction mixture was stirred for 1 h. Then, the reaction mixture was evaporated under reduced pressure and the solid was further triturated with ether (3 mL). The solid was filtered and washed with ice cooled ether (2 mL) and dried under reduced pressure to give N-[6-[2-(dimethylamino)-[1,2,4]triazolo[1,5-a]pyridin-7-yl]-2-methoxy-3-pyridyl]-5-methyl-3-phenyl-isoxazole-4-carboxamide hydrochloride (25 mg, 34% yield) as a colourless solid. ¹H NMR (600 MHZ, Chloroform-d) δ8.80 (d, J=7.7 Hz, 1H), 8.53-8.45 (m, 1H), 8.35 (d, J=6.7 Hz, 1H), 7.88 (d, J=6.7 Hz, 1H), 7.85 (s, 1H), 7.67-7.62 (m, 3H), 7.60 (d, J=7.4 Hz, 2H), 7.55 (d, J=8.2 Hz, 1H), 3.73 (s, 3H), 3.31 (s, 6H), 2.84 (s, 3H). m/z 470.0 [M+H for free base]⁺.

### Example 132 N-[2-Methoxy-6-(3-methylimidazol-4-yl)-3-pyridyl]-5-methyl-3-phenyl-isoxazole-4-carboxamide

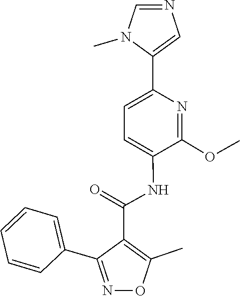 [SMILES: COc1nc(-c2cncn2C)ccc1NC(=O)c1c(-c2ccccc2)noc1C]

A 5 mL microwave vial was charged with Intermediate 1 (250 mg, 0.64 mmol), 1-methyl-5-(4,4,5,5-tetramethyl-1,3,2-dioxaborolan-2-yl)-1H-imidazole (161 mg, 0.77 mmol), Pd-118 (42 mg, 0.06 mmol), KOAc (253 mg, 2.58 mmol), 1,4-dioxane (2 mL) and water (0.2 mL). The vial was sealed, purged with nitrogen and heated at 80° C. for 16 h, then at room temperature for 5 days. The reaction mixture was concentrating under reduced pressure. The residue was redissolved in EtOAc (20 mL), washed with water (10 mL), brine (10 mL), dried over MgSO₄, filtered and concentrated under reduced pressure. The residue was adsorbed onto silica and purified by flash column chromatography (4 g silica, elution with a 0-5% MeOH/DCM gradient) to afford N-[2-methoxy-6-(3-methylimidazol-4-yl)-3-pyridyl]-5-methyl-3-phenyl-isoxazole-4-carboxamide (108 mg, 41% yield) as an off-white solid. ¹H NMR (500 MHZ, Chloroform-d) δ8.67 (d, J=8.2 Hz, 1H), 7.70 (s, 1H), 7.66-7.60 (m, 3H), 7.60-7.54 (m, 2H), 7.45 (s, 1H), 7.37 (s, 1H), 7.16 (d, J=8.2 Hz, 1H), 3.93 (s, 3H), 3.63 (s, 3H), 2.83 (s, 3H). m/z 390.0 [M+H]⁺.

### Example 133 N-[5-(5-Fluoro-6-oxo-1H-pyridin-3-yl)-3-methoxy-pyrazin-2-yl]-5-methyl-3-phenyl-isoxazole-4-carboxamide

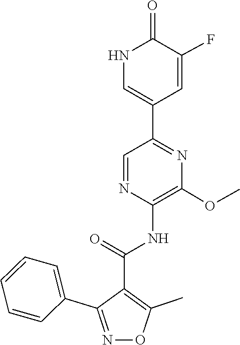 [SMILES: COc1nc(-c2c[nH]c(=O)c(F)c2)cnc1NC(=O)c1c(-c2ccccc2)noc1C]

A microwave vial was charged with Intermediate 13 (300 mg, 0.77 mmol), 5-bromo-3-fluoro-1H-pyridin-2-one (156 mg, 0.77 mmol), Na₂CO₃ (408 mg, 3.85 mmol) and Pd(dppf)Cl₂ (56 mg, 0.077 mmol). The reaction mixture was then evacuated and backfilled with nitrogen three times before being irradiated under microwave at 120° C. for 40 min. The reaction mixture was concentrated to dryness. The residue was taken up in DCM (20 mL) and the organics were washed with water (20 mL) and then brine (20 mL). The collected organics were dried over MgSO₄, filtered and concentrated to dryness under reduced pressure. The residue was purified by flash silica column chromatography on an ISCO system (elution with 4% MeOH/DCM gradient) yielded N-[5-(5-fluoro-6-oxo-1H-pyridin-3-yl)-3-methoxy-pyrazin-2-yl]-5-methyl-3-phenyl-isoxazole-4-carboxamide (18 mg, 5% yield). ¹H NMR (500 MHZ, Chloroform-d) δ13.27 (s, 1H), 8.19 (s, 1H), 7.98 (s, 1H), 7.91 (s, 1H), 7.80 (d, J=10.6 Hz, 1H), 7.68-7.61 (m, 3H), 7.58 (d, J=7.2 Hz, 1H), 3.71 (s, 3H), 2.85 (s, 3H). m/z 422.1 [M+H]⁺.

### Example 134 Ethyl 2-[5-[6-methoxy-5-[(5-methyl-3-phenyl-isoxazole-4-carbonyl)amino]-2-pyridyl]-2-methyl-imidazol-1-yl] acetate

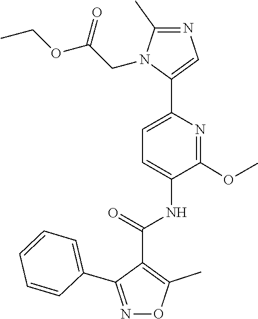 [SMILES: CCOC(=O)Cn1c(-c2ccc(NC(=O)c3c(-c4ccccc4)noc3C)c(OC)n2)cnc1C]

### Step 1

A stirring solution of 4-bromo-2-methyl-1H-imidazole (2.00 g, 12.4 mmol) in acetone (20 mL) was treated with K₂CO₃ (2.58 g, 18.6 mmol) and ethyl chloroacetate (1.33 mL, 12.4 mmol). The reaction mixture was stirred at 60° C. for 15 h, then at room temperature for 5 days. The reaction mixture was filtered and the filtrate concentrated under reduced pressure. The residue was adsorbed onto silica and purified by flash column chromatography (4 g silica, elution with 0-100% EtOAc/PE gradient) to afford ethyl 2-(5-bromo-2-methyl-imidazol-1-yl)acetate (890 mg, 28% yield) as an off-white solid. ¹H NMR (500 MHZ, Chloroform-d) δ6.80 (s, 1H), 4.54 (s, 2H), 4.25 (q, J=7.1 Hz, 2H), 2.33 (s, 3H), 1.29 (t, J=7.1 Hz, 3H). m/z 246.8 [M+H, ⁷⁹Br]⁺, 248.8 [M+H, ⁸¹Br]⁺.

### Step 2

A 5 mL microwave vial was charged with Intermediate 7 (100 mg, 0.26 mmol), ethyl 2-(5-bromo-2-methyl-imidazol-1-yl)acetate (76 mg, 0.31 mmol), Pd-118 (17 mg, 0.03 mmol), KOAc (101 mg, 1.03 mmol), 1,4-dioxane (1.6 mL) and water (0.16 mL). The vessel was sealed, purged with nitrogen and heated at 80° C. for 16 h. The solvent was removed under reduced pressure and the residue was redissolved in EtOAc (20 mL) and washed with water (10 mL), brine (10 mL), dried over MgSO₄, filtered and concentrated under reduced pressure. The residue was adsorbed onto silica and purified by flash column chromatography (4 g silica, elution with a 0-5% MeOH/DCM gradient) to afford ethyl 2-[5-[6-methoxy-5-[(5-methyl-3-phenyl-isoxazole-4-carbonyl)amino]-2-pyridyl]-2-methyl-imidazol-1-yl] acetate (15 mg, 12% yield) as a brown solid. ¹H NMR (500 MHZ, Chloroform-d) δ8.65 (d, J=8.2 Hz, 1H), 7.71 (s, 1H), 7.65-7.62 (m, 2H), 7.62-7.58 (m, 1H), 7.58-7.53 (m, 2H), 7.48 (d, J=8.2 Hz, 1H), 7.34 (s, 1H), 4.61 (s, 2H), 4.24 (q, J=7.2 Hz, 2H), 3.62 (s, 3H), 2.82 (s, 3H), 2.40 (s, 3H), 1.29 (t, J=7.1 Hz, 3H). m/z 476.0 [M+H]⁺.

### Example 135 N-(6-(Imidazo[1,2-a]pyrazin-3-yl)-2-methoxypyridin-3-yl)-5-methyl-3-phenylisoxazole-4-carboxamide

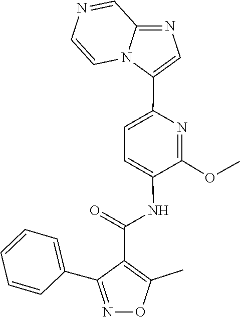 [SMILES: COc1nc(-c2cnc3cnccn23)ccc1NC(=O)c1c(-c2ccccc2)noc1C]

A 20 mL microwave vial was charged with Intermediate 7 (150 mg, 0.34 mmol), 3-bromoimidazo[1,2-a]pyrazine (75 mg, 0.38 mmol), KF (60 mg, 1.03 mmol), 1,4-dioxane (4.5 mL) and water (1.5 mL). The reaction mixture was degassed with nitrogen for 5 mins. Pd-118 (23 mg, 0.03 mmol) was quickly added and the mixture further degassed with nitrogen before stirring at rt overnight. The reaction mixture was then concentrated under reduced pressure. The dark residue dissolved in DCM (20 mL) and washed with water (2×10 mL). The organic layer was dried over MgSO₄, filtered and concentrated under reduced pressure. The residue was purified by flash column chromatography (12 g, silica, 0-100% EtOAc/PE over 40 CV's). CV's containing product were combined and evaporated under reduced pressure. The precipitate was then dried under vacuum at 40° C. to afford N-(6-(imidazo[1,2-a]pyrazin-3-yl)-2-methoxypyridin-3-yl)-5-methyl-3-phenylisoxazole-4-carboxamide (48.4 mg, 30% yield) as a tan solid. ¹H NMR (500 MHZ, Chloroform-d) δ9.44 (dd, J=4.9 & 1.5 Hz, 1H), 9.14 (d, J=1.5 Hz, 1H), 8.80 (d, J=8.3 Hz, 1H), 8.19 (s, 1H), 7.94 (d, J=4.7 Hz, 1H), 7.78 (br s, 1H), 7.70-7.58 (m, 5H), 7.39 (d, J=8.2 Hz, 1H), 3.75 (s, 3H), 2.85 (s, 3H). m/z 427.1 [M+H]⁺.

### Example 136 N-(6-(Imidazo[1,2-a]pyrimidin-3-yl)-2-methoxypyridin-3-yl)-5-methyl-3-phenylisoxazole-4-carboxamide

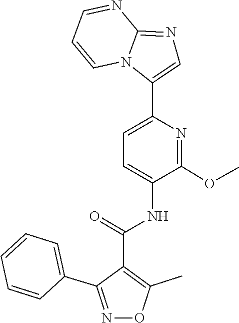 [SMILES: COc1nc(-c2cnc3ncccn23)ccc1NC(=O)c1c(-c2ccccc2)noc1C]

A 10 mL microwave vial was charged with Intermediate 7 (80 mg, 0.19 mmol), 3-bromoimidazo[1,2-a]pyrimidine (41 mg, 0.21 mmol), KF (33 mg, 0.57 mmol), 1,4-dioxane (3 mL) and water (1 mL). The reaction mixture was degassed with nitrogen for 5 mins. Pd-118 (12 mg, 0.02 mmol) was quickly added and the mixture further degassed with nitrogen before stirring at rt overnight. The reaction mixture was then concentrated under reduced pressure. The dark residue dissolved in DCM (20 mL) and washed with water (2×10 mL). The organic layer was dried over MgSO₄, filtered and concentrated under reduced pressure. The residue was purified by flash column chromatography (12 g, silica, 0-100% EtOAc/PE over 40 CV's).

CV's containing product were combined and evaporated under reduced pressure. The residue was triturated with hot EtOAc (3 mL) and the precipitate dried under vacuum at 40° C. to afford N-(6-(imidazo[1,2-a]pyrimidin-3-yl)-2-methoxypyridin-3-yl)-5-methyl-3-phenylisoxazole-4-carboxamide (25.9 mg, 29% yield) as a cream solid. ¹H NMR (500 MHZ, Chloroform-d) δ9.90 (dd, J=6.9 & 2.1 Hz, 1H), 8.78 (d, J=8.3 Hz, 1H), 8.58 (dd, J=4.1 & 2.0 Hz, 1H), 8.23 (s, 1H), 7.74 (br s, 1H), 7.68-7.58 (m, 5H), 7.37 (d, J=8.3 Hz, 1H), 6.94 (dd, J=7.0 & 4.1 Hz, 1H), 3.73 (s, 3H), 2.85 (s, 3H). m/z 427.1 [M+H]⁺.

### Example 137 N-(2-Methoxy-6-(7-methyl-5,6,7,8-tetrahydroimidazol[1,2-a]pyrazin-3-yl)pyridin-3-yl)-5-methyl-3-phenylisoxazole-4-carboxamide

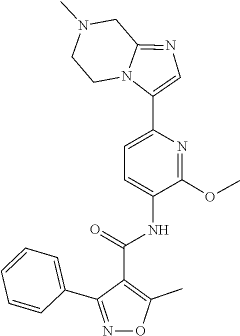 [SMILES: COc1nc(-c2cnc3n2CCN(C)C3)ccc1NC(=O)c1c(-c2ccccc2)noc1C]

### Step 1

A 20 mL microwave vial was charged with Intermediate 7 (0.40 g, 0.92 mmol), tert-butyl 3-bromo-5,6-dihydroimidazo[1,2-a]pyrazine-7 (8H)-carboxylate (0.31 g, 1.01 mmol), KF (160 mg, 2.76 mmol), 1,4-dioxane (9 mL) and water (3 mL). The reaction mixture was degassed with nitrogen for 5 mins. Pd-118 (60 mg, 0.09 mmol) was quickly added and the mixture further degassed with nitrogen before stirring at 50° C. overnight. The reaction mixture was then concentrated under reduced pressure. The dark residue dissolved in DCM (20 mL) and washed with water (2×10 mL). The organic layer was dried over MgSO₄, filtered and concentrated under reduced pressure. The residue was purified by flash column chromatography (12 g, silica, 0-100% EtOAc/PE+1% Et₃N over 40 CV's). CV's containing product were combined and evaporated under reduced pressure. The precipitate was then dried under vacuum at 40° C. to afford tert-butyl 3-(6-methoxy-5-(5-methyl-3-phenylisoxazole-4-carboxamido)pyridin-2-yl)-5,6-dihydroimidazo[1,2-a]pyrazine-7 (8H)-carboxylate (0.21 g, 42% yield) as a yellow solid. ¹H NMR (500 MHZ, Chloroform-d) δ8.66 (d, J=8.2 Hz, 1H), 7.69 (br s, 1H), 7.66-7.60 (m, 3H), 7.60-7.55 (m, 2H), 7.36 (s, 1H), 7.13 (d, J=8.2 Hz, 1H), 4.74 (s, 2H), 4.39 (t, J=5.4 Hz, 2H), 3.82 (t, J=5.5 Hz, 2H), 3.61 (s, 3H), 2.83 (s, 3H), 1.49 (s, 9H). m/z 531.2 [M+H]⁺.

### Step 2

A 10 mL microwave vial was charged with tert-butyl 3-(6-methoxy-5-(5-methyl-3-phenylisoxazole-4-carboxamido)pyridin-2-yl)-5,6-dihydroimidazo[1,2-a]pyrazine-7 (8H)-carboxylate (50 mg, 0.09 mmol), paraformaldehyde (11 mg, 0.38 mmol) and formic acid (2 mL). The reaction mixture was heated to 95° C. and stirred for 2 h. The reaction mixture was cooled, diluted with DCM (15 mL) and washed sequentially with 2 M NaOH (2×15 mL), water (2×15 mL) and brine (1×15 mL). The organic layer was dried over MgSO₄, filtered and concentrated under reduced pressure. The residue was triturated with EtOAc/PE (1:3 ratio) and the precipitate dried under vacuum at 40° C. to afford N-(2-methoxy-6-(7-methyl-5,6,7,8-tetrahydroimidazol[1,2-a]pyrazin-3-yl)pyridin-3-yl)-5-methyl-3-phenylisoxazole-4-carboxamide (28 mg, 64% yield) as a brown solid. ¹H NMR (500 MHZ, Chloroform-d) δ8.67 (d, J=8.2 Hz, 1H), 7.70 (br s, 1H), 7.60-7.69 (m, 3H), 7.62-7.57 (m, 2H), 7.35 (s, 1H), 7.15 (d, J=8.2 Hz, 1H), 4.43 (t, J=5.6 Hz, 2H), 3.74 (s, 2H), 3.63 (s, 3H), 2.83-2.87 (s, 3H & t, J=5.2 Hz, 2H), 2.52 (s, 3H). m/z 445.2 [M+H]⁺.

### Example 138 N-(2-Methoxy-6-(5,6,7,8-tetrahydroimidazo[1,2-a]pyrazin-3-yl)pyridin-3-yl)-5-methyl-3-phenylisoxazole-4-carboxamide hydrochloride

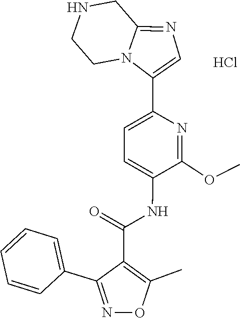 [SMILES: COc1nc(-c2cnc3n2CCNC3)ccc1NC(=O)c1c(-c2ccccc2)noc1C ; Cl]

A 10 mL microwave vial was charged with tert-butyl 3-(6-methoxy-5-(5-methyl-3-phenylisoxazole-4-carboxamido)pyridin-2-yl)-5,6-dihydroimidazo[1,2-a]pyrazine-7 (8H)-carboxylate (synthesised according to Example 137) (75 mg, 0.14 mmol), HCl (2 M in ether, 0.71 mL, 1.41 mmol) and DCM (3 mL). The reaction mixture was stirred at rt overnight. The reaction mixture was concentrated under reduced pressure and the precipitate dried under vacuum at 40° C. to afford N-(2-methoxy-6-(5,6,7,8-tetrahydroimidazo[1,2-a]pyrazin-3-yl)pyridin-3-yl)-5-methyl-3-phenylisoxazole-4-carboxamide hydrochloride (58 mg, 86% yield) as a light yellow solid. ¹H NMR (500 MHZ, DMSO-d₆) δ9.91 (br s, 2H), 9.60 (s, 1H), 8.35 (br s, 1H), 7.90 (br s, 1H), 7.72 (d, J=6.8 Hz, 2H), 7.61-7.52 (m, 3H), 7.49 (d, J=8.1 Hz, 1H), 4.74 (t, J=5.8 Hz, 2H), 4.57 (s, 2H), 3.88 (s, 3H), 3.66 (t, J=5.8 Hz, 2H), 2.66 (s, 3H). m/z 431.2 [M+H for free base]⁺.

### Example 139 N-[6-(4-Cyanoimidazol-1-yl)-2-methoxy-3-pyridyl]-5-methyl-3-phenyl-isoxazole-4-carboxamide

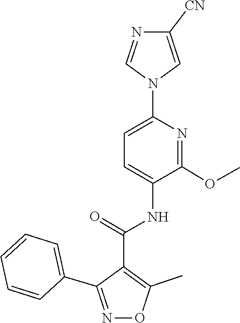 [SMILES: COc1nc(-n2cnc(C#N)c2)ccc1NC(=O)c1c(-c2ccccc2)noc1C]

A 5 mL microwave vial was charged with Intermediate 1 (100 mg, 0.26 mmol), K₂CO₃ (356 mg, 2.58 mmol), L-proline (15 mg, 0.13 mmol), copper(I) iodide (25 mg, 0.13 mmol), 1H-imidazole-4-carbonitrile (29 mg, 0.31 mmol) and DMSO (2 mL). The vial was sealed and degassed before heated at 80° C. for 16 h. The reaction was partitioned between water (20 mL) and EtOAc (30 mL). The phases were separated and the organic layer was washed with brine (20 mL), dried over MgSO₄, filtered and concentrated under reduced pressure. The residue was purified by flash column chromatography (4 g silica, elution with a 0-90% EtOAc/PE gradient) to afford N-[6-(4-cyanoimidazol-1-yl)-2-methoxy-3-pyridyl]-5-methyl-3-phenyl-isoxazole-4-carboxamide (14 mg, 12% yield) as a pale yellow solid. ¹H NMR (600 MHz, Chloroform-d) δ8.85 (d, J=8.3 Hz, 1H), 8.17 (s, 1H), 8.04 (s, 1H), 7.71 (s, 1H), 7.66-7.56 (m, 5H), 6.94 (d, J=8.3 Hz, 1H), 3.67 (s, 3H), 2.83 (s, 3H). m/z 401.0 [M+H]⁺.

### Example 140 N-[2-Methoxy-6-[4-(trifluoromethyl) imidazol-1-yl]-3-pyridyl]-5-methyl-3-phenyl-isoxazole-4-carboxamide

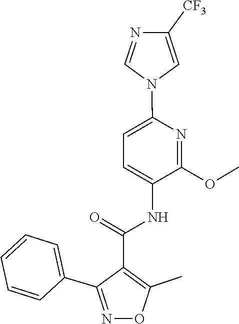 [SMILES: COc1nc(-n2cnc(C(F)(F)F)c2)ccc1NC(=O)c1c(-c2ccccc2)noc1C]

A 5 mL microwave vial was charged with Intermediate 1 (100 mg, 0.26 mmol), K₂CO₃ (356 mg, 2.58 mmol), L-proline (15 mg, 0.13 mmol), copper(I) iodide (25 mg, 0.13 mmol), 4-(trifluoromethyl)-1H-imidazole (42 mg, 0.31 mmol) and DMSO (2 mL). The vial was sealed and degassed before heated at 80° C. for 16 h. The reaction was partitioned between water (20 mL) and EtOAc (30 mL). The phases were separated and the organic layer was washed with brine (20 mL), dried over MgSO₄, filtered and concentrated under reduced pressure. The residue was purified by flash column chromatography (4 g silica, elution with a 0-90% EtOAc/PE gradient) to afford N-[2-methoxy-6-[4-(trifluoromethyl) imidazol-1-yl]-3-pyridyl]-5-methyl-3-phenyl-isoxazole-4-carboxamide (49 mg, 41% yield) as a brown solid. ¹H NMR (600 MHZ, Chloroform-d) δ8.83 (d, J=8.3 Hz, 1H), 8.18 (s, 1H), 7.84 (s, 1H), 7.70 (s, 1H), 7.66-7.60 (m, 3H), 7.58 (t, J=7.5 Hz, 2H), 6.93 (d, J=8.3 Hz, 1H), 3.67 (s, 3H), 2.83 (s, 3H). m/z 443.9 [M+H]⁺.

### Example 141 3-(4-Fluorophenyl)-N-(6-imidazol-1-yl-2-methoxy-3-pyridyl)-5-methyl-isoxazole-4-carboxamide

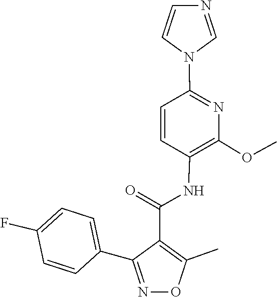 [SMILES: COc1nc(-n2ccnc2)ccc1NC(=O)c1c(-c2ccc(F)cc2)noc1C]

### Step 1—Synthesis of Intermediate 12-N-(6-Bromo-2-methoxy-3-pyridyl)-3-(4-fluorophenyl)-5-methyl-isoxazole-4-carboxamide

To solution of 6-bromo-2-methoxy-3-pyridinamine (2.00 g, 9.85 mmol) in DMF (40 mL) were added HATU (4.49, 11.8 mmol) and Hünig's base (5.15 mL, 29.55 mmol). The reaction mixture was stirred at rt for 30 min before adding 3-(4-fluorophenyl)-5-methyl-isoxazole-4-carboxylic acid (2.18 g, 9.86 mmol). The reaction mixture was then heated at 80° C. o/n. The reaction mixture was concentrated to dryness and the residue taken up in EtOAc (25 mL). The organic layer was washed with water (2×5 mL), then saturated brine solution (10 mL).

The organics were dried over MgSO₄, filtered and concentrated to dryness under reduced pressure. The crude was then purified by flash column chromatography (eluting with 70-80% PE/EtOAc). The desired fractions were concentrated to dryness under reduced pressure. The product obtained was triturated with MeOH (3 mL) followed by filtration to give N-(6-bromo-2-methoxy-3-pyridyl)-3-(4-fluorophenyl)-5-methyl-isoxazole-4-carboxamide (1.3 g, 33% yield) as a colourless solid. ¹H NMR (600 MHZ, Chloroform-d) δ8.53 (d, J=8.2 Hz, 1H), 7.68-7.55 (m, 2H), 7.52 (s, 1H), 7.31-7.20 (m, 2H), 7.03 (d, J=8.2 Hz, 1H), 3.68 (s, 3H), 2.79 (s, 3H). m/z 405.9 [M+H]⁺.

### Step 2

To a solution of Intermediate 12 (150 mg, 0.37 mmol) in DMSO (3 mL) were added L-proline (21.2 mg, 0.18 mmol), copper(I) iodide (35 mg, 0.18 mmol), imidazole (30 mg, 0.44 mmol) and K₂CO₃ (153 mg, 1.10 mmol). The reaction mixture was evacuated and backfilled with nitrogen three times, then heated at 60° C. o/n. The reaction mixture was diluted with EtOAc (20 mL) and was filtered over celite. The organic layer was then washed with water (20 mL) and brine (20 mL), dried over MgSO₄, filtered and concentrated under reduced pressure.

The crude was then purified by flash silica column chromatography on an ISCO system (elution with 3% MeOH/DCM gradient) to give 3-(4-fluorophenyl)-N-(6-imidazol-1-yl-2-methoxy-3-pyridyl)-5-methyl-isoxazole-4-carboxamide (38 mg, 25% yield). ¹H NMR (600 MHz, Chloroform-d) δ 8.78 (d, J=8.4 Hz, 1H), 8.21 (d, J=1.2 Hz, 1H), 7.68-7.57 (m, 3H), 7.50 (d, J=1.4 Hz, 1H), 7.28 (t, J=8.5 Hz, 2H), 7.15 (d, J=1.3 Hz, 1H), 6.90 (d, J=8.3 Hz, 1H), 3.74 (s, 3H), 2.82 (s, 3H). m/z 394.1 [M+H]⁺.

### Example 142 N-[5-(2-Ethoxypyrimidin-5-yl)-3-methoxy-pyrazin-2-yl]-5-methyl-3-phenyl-isoxazole-4-carboxamide

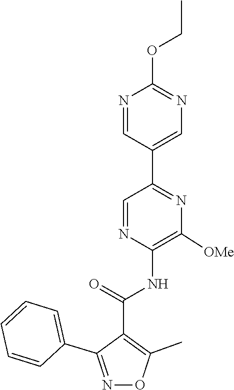 [SMILES: CCOc1ncc(-c2cnc(NC(=O)c3c(-c4ccccc4)noc3C)c(OC)n2)cn1]

### Step 1

Sodium (392 mg, 17.1 mmol) was added portion-wise to ice-cold EtOH (10 mL). Upon completion of addition, the cooling bath was removed and the consumption of sodium monitored by eye. Once the sodium was consumed (45 min), 5-bromo-2-chloropyrimidine (2.00 g, 10.34 mmol) was added and the mixture left to stir at rt for 1 h. The reaction mixture was diluted with EtOAc (20 mL), washed with water (20 mL) and brine (20 mL). The organic extract was dried over MgSO₄, filtered and concentrated under reduced pressure to give 5-bromo-2-ethoxy-pyrimidine (2.04 g, 97% yield) as a colourless solid. ¹H NMR (500 MHZ, Chloroform-d) δ8.49 (s, 2H), 4.37 (q, J=7.1 Hz, 2H), 1.40 (t, J=7.1 Hz, 3H). m/z 204.9 [M+H, ⁸¹Br]⁺.

### Step 2—Synthesis of Intermediate 13-N-[3-Methoxy-5-(4,4,5,5-tetramethyl-1,3,2-dioxaborolan-2-yl) pyrazin-2-yl]-5-methyl-3-phenyl-isoxazole-4-carboxamide

To a microwave vial under an inert atmosphere was added Intermediate 6 (0.50 g, 1.28 mmol), B₂Pin₂ (1.31 g, 5.14 mmol) and 1,4-dioxane (12 mL). The solution was degassed with nitrogen for 15 min before Pd (dppf)₂Cl₂. DCM complex (105 mg, 0.13 mmol) and KOAc (378 mg, 3.85 mmol) were quickly added. The mixture was again degassed with nitrogen for 15 min before being heated to 80° C. for 1 h. The reaction mixture was allowed to cool to rt and was diluted with EtOAc (30 mL). The solution was washed with water (20 mL), dried over MgSO₄, filtered and concentrated under reduced pressure to give the crude product. PE (80 mL) was added and the suspension triturated overnight. After allowing the suspension to settle, the supernatant was removed by pipette and the procedure repeated (this time stirring for only 10 min). The solid obtained was dried under reduced pressure and made into smaller particles using a spatula. Diethyl ether (80 mL) was added and the mixture heated with swirling to boiling point using a heat gun. The suspension was filtered (gravity, filter paper) whilst hot and the filtrate concentrated under reduced pressure to give N-[3-methoxy-5-(4,4,5,5-tetramethyl-1,3,2-dioxaborolan-2-yl) pyrazin-2-yl]-5-methyl-3-phenyl-isoxazole-4-carboxamide (483 mg 82% yield). ¹H NMR (500 MHZ, Chloroform-d) δ8.32 (s, 1H), 8.01 (s, 1H), 7.64-7.56 (m, 3H), 7.55-7.50 (m, 2H), 3.69 (s, 3H), 2.82 (s, 3H), 1.33 (s, 12H). m/z 354.9 [M+H for boronic acid]⁺.

### Step 3

A solution of 5-bromo-2-ethoxy-pyrimidine (90 mg, 0.44 mmol), Intermediate 13 (200 mg, 0.43 mmol) and KF (64 mg, 1.10 mmol) in 1,4-dioxane (8 mL) and water (2 mL) was degassed with nitrogen for 15 min. Pd-118 (24 mg, 0.037 mmol) was added quickly, the solution degassed for a further 10 min before being left to stir at rt overnight. The reaction mixture was diluted with EtOAc (20 mL), washed with water (3×20 mL) and brine (20 mL). The organic extract was dried over MgSO₄, filtered and concentrated under reduced pressure to give the crude product, which was purified by automated column chromatography (gradient over 25 CV from 0-50% EtOAc/n-hexane, followed by a fast gradient to 100% EtOAc) to give N-[5-(2-ethoxypyrimidin-5-yl)-3-methoxy-pyrazin-2-yl]-5-methyl-3-phenyl-isoxazole-4-carboxamide (111 mg, 59% yield) as a light yellow solid. ¹H NMR (500 MHz, Chloroform-d) δ 8.99 (s, 2H), 8.31 (s, 1H), 7.95 (s, 1H), 7.66-7.60 (m, 3H), 7.59-7.54 (m, 2H), 4.48 (q, J=7.1 Hz, 2H), 3.75 (s, 3H), 2.85 (s, 3H), 1.46 (t, J=7.1 Hz, 3H). m/z 433.1 [M+H]⁺.

### Example 143 N-(6-(7-Acetyl-5,6,7,8-tetrahydroimidazo[1,2-a]pyrazin-3-yl)-2-methoxypyridin-3-yl)-5-methyl-3-phenylisoxazole-4-carboxamide

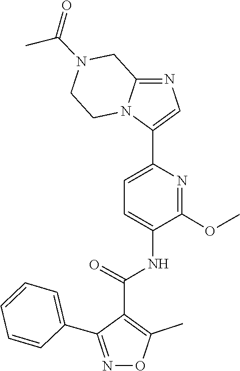 [SMILES: COc1nc(-c2cnc3n2CCN(C(C)=O)C3)ccc1NC(=O)c1c(-c2ccccc2)noc1C]

A dry 10 mL microwave vial was charged with Example 138 (30 mg, 0.06 mmol), Et₃N (20 μL, 0.18 mmol) and anhydrous DCM (2 mL) under nitrogen. Acetyl chloride (1 M in DCM, 70 μL, 0.07 mmol) was then added. The reaction mixture was stirred at rt overnight. The reaction mixture was diluted with DCM (10 mL) and washed sequentially with saturated NaHCO₃ solution (2×10 mL), water (2×10 mL) and brine (10 mL). The organic layer was dried over MgSO₄, filtered and concentrated under reduced pressure. The residue was purified by flash column chromatography (4 g, silica, 0-10% MeOH/DCM+1% Et₃N over 30 CV's). CV's containing product were combined and evaporated under reduced pressure. The precipitate was then dried under vacuum at 40° C. to afford N-(6-(7-acetyl-5,6,7,8-tetrahydroimidazo[1,2-a]pyrazin-3-yl)-2-methoxypyridin-3-yl)-5-methyl-3-phenylisoxazole-4-carboxamide (8.7 mg, 29% yield) as a colourless solid. ¹H NMR (500 MHZ, Chloroform-d) δ8.68 (d, J=8.1 Hz, 1H), 7.70 (br s, 1H), 7.66-7.61 (m, 3H), 7.61-7.55 (m, 2H), 7.37 (br s, 1H), 7.14 (d, J=8.2 Hz, 1H), 4.81 (s, 2H), 4.40 (t, J=5.6 Hz, 2H), 4.04-3.97 (m, 2H), 3.60 (s, 3H), 2.83 (s, 3H), 2.20 (s, 3H). Compound exists as a mixture of rotamers in ˜3:1 ratio in chloroform-d and ˜1:1 ratio in DMSO-de that was confirmed in a variable temperature experiment in DMSO-d₆. m/z 473.1 [M+H]⁺.

### Example 144 N-[5-[2-[(Dimethylamino)methyl]pyrimidin-5-yl]-3-methoxy-pyrazin-2-yl]-5-methyl-3-phenyl-isoxazole-4-carboxamide

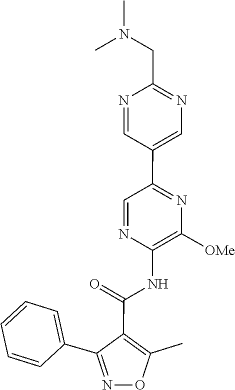 [SMILES: COc1nc(-c2cnc(CN(C)C)nc2)cnc1NC(=O)c1c(-c2ccccc2)noc1C]

A solution of Intermediate 9 (119 mg, 0.55 mmol), Intermediate 13 (0.300 g, 0.687 mmol) and KF (96 mg, 1.65 mmol) in 1,4-dioxane (12 mL) and water (3 mL) was degassed with nitrogen for 15 min. Pd-118 (36 mg, 0.055 mmol) was added quickly, the solution degassed for a further 10 min before being left to stir at rt overnight. Following degassing, additional KF (96 mg, 1.65 mmol) and Pd-118 (36 mg, 0.055 mmol) were added and the mixture heated at 35° C. overnight. A further portion of KF (96 mg, 1.65 mmol) and Pd-118 (36 mg, 0.055 mmol) were added and the mixture left to stir for 2 days. The reaction mixture was diluted with EtOAc (20 mL), washed with water (3×20 mL) and brine (20 mL). The organic extract was dried over MgSO₄, filtered and concentrated under reduced pressure to give the crude product, which was purified by automated column chromatography (gradient over 35 CV from 0-10% MeOH/DCM) to give N-[5-[2-[(dimethylamino)methyl]pyrimidin-5-yl]-3-methoxy-pyrazin-2-yl]-5-methyl-3-phenyl-isoxazole-4-carboxamide (82 mg, 33% yield) as a light brown solid. ¹H NMR (500 MHZ, Chloroform-d) δ9.20 (s, 2H), 8.41 (s, 1H), 8.02 (s, 1H), 7.66 (m, 3H), 7.60 (d, J=7.4 Hz, 2H), 3.82 (s, 2H), 3.78 (s, 3H), 2.87 (s, 3H), 2.40 (s, 6H). m/z 446.1 [M+H]⁺.

### Example 145 N-[3-Methoxy-5-[2-(methylamino)pyrimidin-5-yl] pyrazin-2-yl]-5-methyl-3-phenyl-isoxazole-4-carboxamide

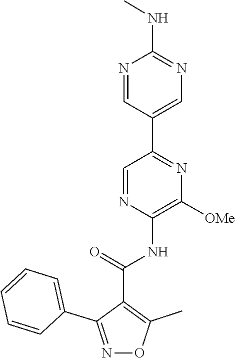 [SMILES: CNc1ncc(-c2cnc(NC(=O)c3c(-c4ccccc4)noc3C)c(OC)n2)cn1]

A solution of Intermediate 13 (358 mg, 0.77 mmol), 5-bromo-N-methyl-pyrimidin-2-amine (145 mg, 0.73 mmol) and KF (127 mg, 2.19 mmol) in 1,4-dioxane (8 mL) and water (2 mL) was degassed with nitrogen for 15 min. Pd-118 (48 mg, 0.073 mmol) was added quickly, the solution degassed for a further 10 min before being left to stir at 35° C. for 2 days. The reaction mixture was diluted with EtOAc (20 mL) and washed with water (30 mL). The organic phase was concentrated directly under reduced pressure without drying over MgSO₄. The resulting solid was washed with diethyl ether (3×30 mL), then with MeOH (40 mL), giving a very fine suspension which was gravity filtered through filter paper. Once dry, the solid was collected and further dried in a vacuum oven at 40° C. for 1 h to give N-[3-methoxy-5-[2-(methylamino)pyrimidin-5-yl] pyrazin-2-yl]-5-methyl-3-phenyl-isoxazole-4-carboxamide (135 mg, 44% yield). ¹H NMR (500 MHZ, DMSO-d₆) δ10.55 (s, 1H), 9.08-8.87 (m, 2H), 8.47 (s, 1H), 7.63 (d, J=7.4 Hz, 2H), 7.61-7.54 (m, 1H), 7.52-7.41 (m, 3H), 3.95 (s, 3H), 2.87 (d, J=4.8 Hz, 3H), 2.61 (s, 3H). m/z 418.1 [M+H]⁺.

### Example 146 N-(6-Imidazol-1-yl-2-methoxy-3-pyridyl)-5-methyl-3-(2-pyridyl) isoxazole-4-carboxamide

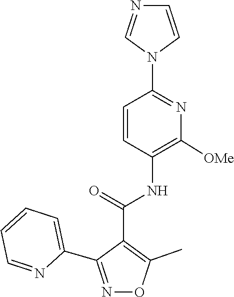 [SMILES: COc1nc(-n2ccnc2)ccc1NC(=O)c1c(-c2ccccn2)noc1C]

### Step 1

To solution of 5-methyl-3-(2-pyridinyl)-1,2-oxazole-4-carboxylic acid (500 mg, 2.45 mmol) in DMF (5 mL) were added HATU (1.12 g, 2.94 mmol) and Hünig's base (1.28 mL, 7.35 mmol). The reaction mixture was stirred at rt for 30 min before 6-bromo-2-methoxy-3-pyridinamine (497 mg, 2.45 mmol) was added. The reaction mixture was then heated at 70° C. overnight. After allowing to cool to rt, the reaction mixture was concentrated to dryness and the residue dissolved in EtOAc (50 mL). The organic layer was washed with water (2×5 mL), brine solution (1×20 mL), dried over MgSO₄ and concentrated under reduced pressure. The crude product was purified by flash column chromatography (eluting with 80% PE/EtOAc) to give N-(6-bromo-2-methoxy-3-pyridyl)-5-methyl-3-(2-pyridyl) isoxazole-4-carboxamide (0.56 g, 56% yield) as a colourless solid. ¹H NMR (500 MHZ, Chloroform-d) δ13.37 (s, 1H), 8.69 (ddd, J=5.0, 1.8, 0.9 Hz, 1H), 8.63 (d, J=8.2 Hz, 1H), 8.23 (dt, J=8.1, 1.1 Hz, 1H), 7.93 (td, J=7.8, 1.8 Hz, 1H), 7.51 (ddd, J=7.6, 5.0, 1.2 Hz, 1H), 7.09 (d, J=8.2 Hz, 1H), 4.13 (s, 3H), 2.86 (s, 3H). m/z 390.9 [M+H, ⁸¹Br]⁺.

### Step 2

To solution of N-(6-bromo-2-methoxy-3-pyridyl)-5-methyl-3-(2-pyridyl) isoxazole-4-carboxamide (150 mg, 0.39 mmol) in DMSO (3 mL) were added K₂CO₃ (160 mg, 1.16 mmol), L-proline (22 mg, 0.19 mmol) and imidazole (32 mg, 0.46 mmol). The mixture was degassed by bubbling nitrogen for 3 min before copper(I) iodide (37 mg, 0.19 mmol) was added and the mixture heated at 80° C. overnight. TLC analysis showed new product formation and LCMS showed required mass for desired product. The reaction mixture was diluted with EtOAc (20 mL) and filtered through a pad of celite. The clear filtrate was washed with water (2×3 mL) and the organic layer dried over MgSO₄, filtered and concentrated under reduced pressure to give the crude product, which was purified using automated column chromatography (2-4% MeOH in DCM) to give N-(6-imidazol-1-yl-2-methoxy-3-pyridyl)-5-methyl-3-(2-pyridyl) isoxazole-4-carboxamide (63 mg, 41% yield) as an off-white solid. ¹H NMR (500 MHZ, Chloroform-d) δ13.47 (s, 1H), 8.87 (d, J=8.3 Hz, 1H), 8.73 (ddd, J=5.0, 1.8, 0.9 Hz, 1H), 8.35 (s, 1H), 8.25 (dt, J=8.0, 1.1 Hz, 1H), 7.95 (td, J=7.8, 1.8 Hz, 1H), 7.64 (s, 1H), 7.53 (ddd, J=7.6, 5.0, 1.2 Hz, 1H), 7.23 (s, 1H), 6.96 (d, J=8.4 Hz, 1H), 4.18 (s, 3H), 2.89 (s, 3H). m/z 377.2 [M+H]⁺.

### Example 147 N-[5-(6-Acetamido-3-pyridyl)-3-methoxy-pyrazin-2-yl]-5-methyl-3-phenyl-isoxazole-4-carboxamide

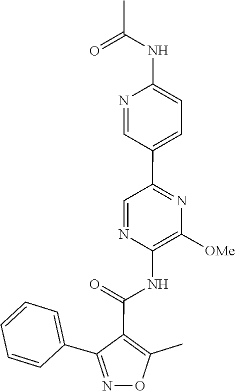 [SMILES: COc1nc(-c2ccc(NC(C)=O)nc2)cnc1NC(=O)c1c(-c2ccccc2)noc1C]

A solution of 2-acetamido-5-bromopyridine (224 mg, 1.04 mmol), Intermediate 13 (478 mg, 1.10 mmol) and KF (181 mg, 3.12 mmol) in 1,4-dioxane (8 mL) and water (2 mL) was degassed with nitrogen for 15 min. Pd-118 (68 mg, 0.12 mmol) was added quickly, the solution degassed for a further 10 min before being left to stir at rt over the weekend. The reaction mixture was diluted with EtOAc (20 mL). It appeared the product was sitting as a suspension in the organic layer and so the organic phase was filtered under gravity (filter paper). Purification by automated column chromatography on silica (12 g, gradient from 0-10% MeOH/DCM over 25 CV) to give N-[5-(6-acetamido-3-pyridyl)-3-methoxy-pyrazin-2-yl]-5-methyl-3-phenyl-isoxazole-4-carboxamide (225 mg, 49% yield) as a colourless solid. ¹H NMR (500 MHZ, Chloroform-d) δ8.79 (d, J=2.3 Hz, 1H), 8.34 (s, 1H), 8.30-8.25 (m, 1H), 8.22-8.16 (m, 2H), 7.97 (s, 1H), 7.66-7.59 (m, 3H), 7.56 (dd, J=8.0, 6.5 Hz, 2H), 3.75 (s, 3H), 2.84 (s, 3H), 2.24 (s, 3H). m/z 445.1 [M+H]⁺.

### Example 148 N-[5-[6-(Hydroxymethyl)-3-pyridyl]-3-methoxy-pyrazin-2-yl]-5-methyl-3-phenyl-isoxazole-4-carboxamide

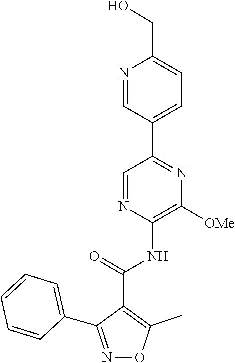 [SMILES: COc1nc(-c2ccc(CO)nc2)cnc1NC(=O)c1c(-c2ccccc2)noc1C]

A solution of (5-bromopyrid-2-yl) methanol (202 mg, 1.08 mmol), Intermediate 13 (490 mg, 1.12 mmol) and KF (188 mg, 3.23 mmol) in 1,4-dioxane (8 mL) and water (2 mL) was degassed with nitrogen for 15 min. Pd-118 (70 mg, 0.11 mmol) was added quickly, the solution degassed for a further 10 min before being left to stir at rt over the weekend. The reaction mixture was diluted with EtOAc (20 mL) and washed with water (4×30 mL). The organic fraction was dried over MgSO₄, filtered and concentrated under reduced pressure to give the crude product, which was purified by automated column chromatography (12 g silica, gradient from 0-10% MeOH/DCM over 25 CV). The fractions containing the product were collected and concentrated under reduced pressure to give N-[5-[6-(hydroxymethyl)-3-pyridyl]-3-methoxy-pyrazin-2-yl]-5-methyl-3-phenyl-isoxazole-4-carboxamide (239 mg, 53% yield) as a light brown powder. ¹H NMR (500 MHZ, Chloroform-d) δ9.09 (s, 1H), 8.39 (s, 1H), 8.16 (d, J=8.2 Hz, 1H), 7.97 (s, 1H), 7.67-7.59 (m, 3H), 7.56 (t, J=7.6 Hz, 2H), 7.34 (d, J=8.2 Hz, 1H), 4.81 (s, 2H), 3.76 (s, 3H), 3.70 (br s), 2.85 (s, 3H). m/z 418.1 [M+H]⁺.

### Example 149 N-[3-Methoxy-5-(1-methyl-6-oxo-3-pyridyl) pyrazin-2-yl]-5-methyl-3-phenyl-isoxazole-4-carboxamide

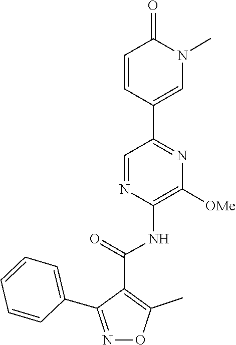 [SMILES: COc1nc(-c2ccc(=O)n(C)c2)cnc1NC(=O)c1c(-c2ccccc2)noc1C]

A solution of Intermediate 13 (450 mg, 1.03 mmol), 5-bromo-1-methyl-2 (1H)-pyridinone (184 mg, 0.98 mmol) and KF (171 mg, 2.94 mmol) in 1,4-dioxane (8 mL) and water (2 mL) was degassed with nitrogen for 15 min. Pd-118 (64 mg, 0.98 mmol) was added quickly, the solution degassed for a further 10 min before being left to stir at rt over the weekend. The reaction mixture was diluted with EtOAc (20 mL) and washed with water (30 mL), which resulted in a troublesome emulsion. For this reason, the aqueous phase was thrice extracted with DCM (3×20 mL) and the combined organics dried over MgSO₄, filtered and concentrated under reduced pressure to give the crude product, which was purified by automated column chromatography on silica (12 g, gradient from 0-10% MeOH/DCM over CV) to give N-[3-methoxy-5-(1-methyl-6-oxo-3-pyridyl) pyrazin-2-yl]-5-methyl-3-phenyl-isoxazole-4-carboxamide (336 mg, 82% yield) as an orange solid. ¹H NMR (500 MHZ, Chloroform-d) δ8.14 (s, 1H), 7.95-7.88 (m, 2H), 7.86 (dd, J=9.5, 2.6 Hz, 1H), 7.68-7.60 (m, 3H), 7.56 (t, J=7.4 Hz, 2H), 6.65 (d, J=9.5 Hz, 1H), 3.72 (s, 3H), 3.63 (s, 3H), 2.84 (s, 3H). m/z 418.1 [M+H]⁺.

### Example 150 N-[5-(5-Aminopyrazin-2-yl)-3-methoxy-pyrazin-2-yl]-5-methyl-3-phenyl-isoxazole-4-carboxamide

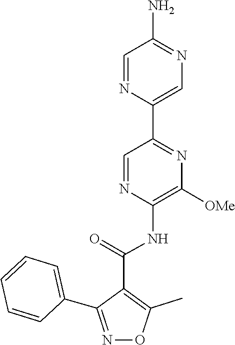 [SMILES: COc1nc(-c2cnc(N)cn2)cnc1NC(=O)c1c(-c2ccccc2)noc1C]

A solution of Intermediate 13 (530 mg, 1.21 mmol), 5-bromopyrazin-2-amine (200 mg, 1.15 mmol) and KF (201 mg, 3.46 mmol) in 1,4-dioxane (8 mL) and (2 mL) was degassed with nitrogen. Pd-118 (75 mg, 0.12 mmol) was added, the solution degassed for a further 10 min before being left to stir at rt for 16 h. The reaction mixture was concentrated under reduced pressure. The resulting solid was washed with DCM (80 mL), boiling hot EtOAc (80 mL), boiling hot MeCN (80 mL) and boiling hot chloroform (80 mL). The combined organic washings were concentrated under reduced pressure to give the crude product, which was partially dissolved in DCM and dry-loaded onto silica. Purification was achieved by automated column chromatography on silica (12 g silica, eluting with 0-10% MeOH/DCM) to give N-[5-(5-aminopyrazin-2-yl)-3-methoxy-pyrazin-2-yl]-5-methyl-3-phenyl-isoxazole-4-carboxamide (132 mg, 27% yield) as a beige solid. ¹H NMR (500 MHZ, DMSO-d₆) δ10.57 (s, 1H), 8.75 (s, 1H), 8.56 (s, 1H), 7.97 (s, 1H), 7.72-7.62 (m, 2H), 7.55-7.39 (m, 3H), 6.94 (s, 2H), 3.98 (s, 3H), 2.61 (s, 3H). m/z 404.1 [M+H]⁺.

### Example 151 N-(6-(1H-Imidazol-1-yl)-2-methoxypyridin-3-yl)-4-methyl-1-phenyl-1H-1,2,3-triazole-5-carboxamide

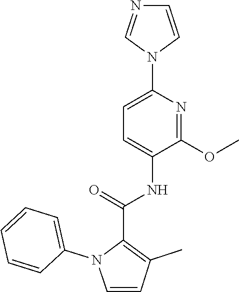 [SMILES: COc1nc(-n2ccnc2)ccc1NC(=O)c1c(C)ccn1-c1ccccc1]

Intermediate 10 (96 mg, 0.22 mmol) was added to a solution of imidazole (36 mg, 0.54 mmol), K₂CO₃ (123 mg, 0.90 mmol) and L-proline (5 mg, 0.04 mmol) in DMSO (4 mL) and the reaction mixture was degassed with nitrogen. Copper (I) iodide (9 mg, 0.04 mmol) was quickly added and the reaction mixture was further degassed before heated at 80° C. for 16 h. The reaction mixture was diluted with EtOAc (15 mL) and filtered through celite. The filtrate was washed with water (2×15 mL) and the organic layer dried over MgSO₄, filtered and concentrated under reduced pressure. The residue was purified by flash column chromatography (4 g silica, eluting with 0-100% EtOAc/PE) to afford N-(6-(1H-imidazol-1-yl)-2-methoxypyridin-3-yl)-4-methyl-1-phenyl-1H-1,2,3-triazole-5-carboxamide (18 mg, 21% yield) as a pink solid. ¹H NMR (500 MHZ, Chloroform-d) δ8.73 (d, J=8.4 Hz, 1H), 8.25 (s, 1H), 7.81 (br s, 1H), 7.55-7.63 (m, 5H), 7.51-7.54 (m, 1H), 7.17 (s, 1H), 6.92 (d, J=8.3 Hz, 1H), 3.91 (s, 3H), 2.72 (s, 3H). m/z 376.1 [M+H]⁺.

### Example 152 N-[6-[4-[(Dimethylamino)methyl] imidazol-1-yl]-2-methoxy-3-pyridyl]-5-methyl-3-phenyl-isoxazole-4-carboxamide Example 153 N-[6-[5-[(Dimethylamino)methyl] imidazol-1-yl]-2-methoxy-3-pyridyl]-5-methyl-3-phenyl-isoxazole-4-carboxamide

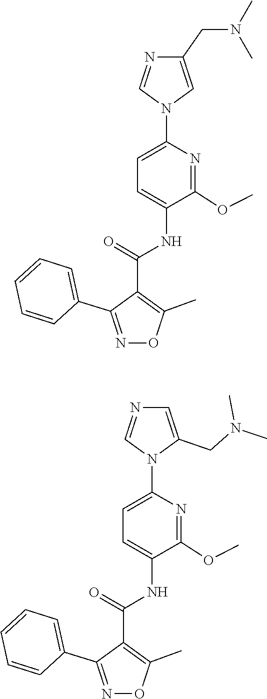 [SMILES: COc1nc(-n2cnc(CN(C)C)c2)ccc1NC(=O)c1c(-c2ccccc2)noc1C ; COc1nc(-n2cncc2CN(C)C)ccc1NC(=O)c1c(-c2ccccc2)noc1C]

### Step 1

A solution of 4-imidazolecarboxaldehyde (266 mg, 2.77 mmol) in THF (10 mL) was treated with dimethylamine (2 M in THF, 2.08 mL, 4.15 mmol) and acetic acid (0.02 mL, 0.3 mmol). The yellow reaction mixture was stirred at rt for 1 h before adding sodium triacetoxyborohydride (880 mg, 4.15 mmol). The yellow mixture was stirred at room temperature for 16 h. The reaction mixture was partitioned between water (20 mL) and EtOAc (20 mL). The layers were separated and the organic layer was washed with brine (10 mL), dried over MgSO₄, filtered and concentrated to give 1-(1H-imidazol-4-yl)-N,N-dimethyl-methanamine (64 mg, 18%) as a yellow oil, which was used in the next step without further purification. ¹H NMR (600 MHZ, DMSO-d₆) δ7.49 (s, 1H), 6.80 (s, 1H), 3.29 (s, 2H), 2.08 (s, 6H). m/z 126.0 [M+H]⁺.

### Step 2

A 5 mL microwave vial was charged with Intermediate 1 (200 mg, 0.52 mmol), K₂CO₃ (712 mg, 5.15 mmol), L-proline (30 mg, 0.26 mmol), copper(I) iodide (49 mg, 0.26 mmol), 1-(1H-imidazol-4-yl)-N, N-dimethyl-methanamine (64 mg, 0.52 mmol) and DMSO (4 mL). The vial was sealed and degassed before heated at 80° C. for 16 h. The reaction was partitioned between water (20 mL) and EtOAc (50 mL) and separated. The organic layer was washed with brine (20 mL), dried over MgSO₄, filtered and concentrated under reduced pressure. The residue was adsorbed onto silica and purified on a Shimadzu MDAP (elution with a 10-95% MeCN/water+0.1% formic acid gradient) to afford two regioisomers. The regiochemistry could not be assigned with confidence so the isomers were arbitrarily assigned as N-[6-[4-[(dimethylamino)methyl] imidazol-1-yl]-2-methoxy-3-pyridyl]-5-methyl-3-phenyl-isoxazole-4-carboxamide (10 mg, 4% yield) as a tan solid and N-[6-[5-[(dimethylamino)methyl] imidazol-1-yl]-2-methoxy-3-pyridyl]-5-methyl-3-phenyl-isoxazole-4-carboxamide (15 mg, 6% yield) as an off-white solid. The isomers were distinguished by differences in their NMR spectra.

N-[6-[4-[(Dimethylamino)methyl] imidazol-1-yl]-2-methoxy-3-pyridyl]-5-methyl-3-phenyl-isoxazole-4-carboxamide: ¹H NMR (600 MHZ, Chloroform-d) δ8.79 (d, J=8.3 Hz, 1H), 8.16 (s, 1H), 7.73 (s, 1H), 7.68 (s, 1H), 7.65-7.60 (m, 3H), 7.60-7.55 (m, 2H), 6.90 (d, J=8.3 Hz, 1H), 4.00 (s, 2H), 3.66 (s, 3H), 2.83 (s, 3H), 2.65 (s, 6H). m/z 433.1 [M+H]⁺. N-[6-[5-[(Dimethylamino)methyl] imidazol-1-yl]-2-methoxy-3-pyridyl]-5-methyl-3-phenyl-isoxazole-4-carboxamide: ¹H NMR (600 MHZ, Chloroform-d) δ8.84 (d, J=8.2 Hz, 1H), 7.96 (s, 1H), 7.75 (s, 1H), 7.66-7.61 (m, 3H), 7.59 (dd, J=8.2, 6.9 Hz, 2H), 7.19 (s, 1H), 7.14 (d, J=8.2 Hz, 1H), 4.03 (s, 2H), 3.63 (s, 3H), 2.83 (s, 3H), 2.30 (s, 6H). m/z 433.1 [M+H]⁺.

### Example 154 N-(6-(1H-Imidazol-1-yl)-2-methoxypyridin-3-yl)-1-methyl-4-phenyl-1H-1,2,3-triazole-5-carboxamide

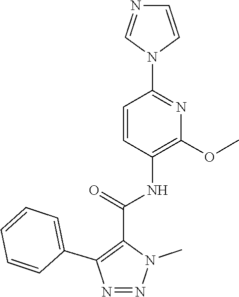 [SMILES: COc1nc(-n2ccnc2)ccc1NC(=O)c1c(-c2ccccc2)nnn1C]

### Step 1

In a microwave vial, 1-methyl-4-phenyl-triazole (0.45 g, 2.8 mmol, synthesised according to WO2012/062687) was dissolved in dry tetrahydrofuran (5 mL) under nitrogen and the reaction mixture was cooled to −78° C. 1.6 M butyl lithium (5.2 mL, 8.4 mmol) in hexane was then added drop-wise, and the resulting yellow solution was stirred for 40 mins. CO₂ (generated from dry ice) was then bubbled into the solution at −78° C. for 1 h using a cannula, over which time the mixture was allowed to warm to room temperature. The reaction mixture was quenched with water (˜3 mL) and the reaction mixture was evaporated under reduced pressure. To the residue was added 2 M NaOH (˜20 mL). The aqueous layer was washed with EtOAc (3×20 mL) and then neutralised with 1 M HCl (˜30 mL). The neutralised extract was then concentrated under vacuum. The precipitate was filtered, washed with additional water (˜10 mL) and petroleum ether (˜10 mL), then dried under vacuum to afford 1-Methyl-4-phenyl-1H-1,2,3-triazole-5-carboxylic acid (0.2 g, 34% yield) as a colourless solid. ¹H NMR (500 MHZ, DMSO-d₆) δ7.68-7.75 (m, 2H), 7.39-7.49 (m, 3H), 4.25 (s, 3H). m/z 204.1 [M+H]⁺.

### Step 2

1-Methyl-4-phenyl-1H-1,2,3-triazole-5-carboxylic acid (197.0 mg, 0.97 mmol), was dissolved in SOCl₂ (0.78 mL, 11 mmol) and the reaction mixture was heated to 65° C. for 16 h. After cooling to rt, excess SOCl₂ was removed under reduced pressure. The residue was dissolved in DCM (5 mL) and then added drop-wise to a solution of 6-bromo-2-methoxy-3-pyridinamine (197 mg, 0.97 mmol) and Et₃N (0.16 mL, 1.2 mmol) in DCM (5 mL) at 0° C. The reaction mixture was left to stir at rt for 2 h. The reaction mixture was diluted with DCM (10 mL) and washed sequentially with water (10 mL), 1 M NaOH (2×10 mL), 1 M HCl (3×10 mL) and brine (10 mL). The organic layer was dried over MgSO₄, filtered and concentrated under reduced pressure to afford N-(6-bromo-2-methoxypyridin-3-yl)-1-methyl-4-phenyl-1H-1,2,3-triazole-5-carboxamide (164 mg, 41% yield) as a brown solid. ¹H NMR (500 MHZ, Chloroform-d) δ8.53 (d, J=8.2 Hz, 1H), 8.03 (br s, 1H), 7.60-7.66 (m, 2H), 7.52-7.58 (m, 3H), 7.07 (d, J=8.2 Hz, 1H), 4.42 (s, 3H), 3.63 (s, 3H). m/z 388.0 [M+H, ⁷⁹Br]⁺, 390.0 [M+H, ⁸¹Br]⁺.

### Step 3

N-(6-Bromo-2-methoxypyridin-3-yl)-1-methyl-4-phenyl-1H-1,2,3-triazole-5-carboxamide (64 mg, 0.33 mmol) was added to a solution of imidazole (27 mg, 0.40 mmol), K₂CO₃ (91 mg, 0.66 mmol) and L-proline (4 mg, 0.04 mmol) in DMSO (3 mL) and the reaction mixture was degassed with nitrogen. Copper (I) iodide (6 mg, 0.04 mmol) was quickly added and the reaction mixture was further degassed before stirring at 80° C. for 16 h. The reaction mixture was diluted with EtOAc (10 mL) and filtered through celite. The filtrate was washed with water (2×10 mL) and the organic layer dried over MgSO₄, filtered and concentrated under reduced pressure. The residue was then triturated with hot EtOAc (3 mL) and the precipitate was collected and dried under vacuum at 40° C. to afford N-(6-(1H-imidazol-1-yl)-2-methoxypyridin-3-yl)-1-methyl-4-phenyl-1H-1,2,3-triazole-5-carboxamide (21 mg, 31% yield) as a colourless solid. ¹H NMR (500 MHZ, Chloroform-d) δ8.80 (d, J=8.3 Hz, 1H), 8.26 (s, 1H), 8.13 (s, 1H), 7.68 (dd, J=6.4 & 2.5 Hz, 2H), 7.57-7.62 (m, 3H), 7.55 (br s, 1H), 7.19 (s, 1H), 6.95 (d, J=8.3 Hz, 1H), 4.46 (s, 3H), 3.72 (s, 3H). m/z 376.1 [M+H]⁺.

### Example 155 N-[5-(6,8-Dihydro-5H-imidazo[2,1-c][1,4]oxazin-3-yl)-3-methoxy-pyrazin-2-yl]-5-methyl-3-phenyl-isoxazole-4-carboxamide

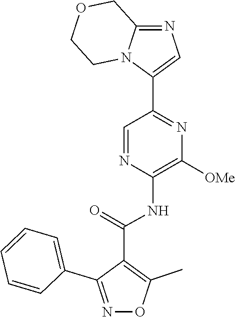 [SMILES: COc1nc(-c2cnc3n2CCOC3)cnc1NC(=O)c1c(-c2ccccc2)noc1C]

A solution of KF (108 mg, 1.86 mmol), 3-bromo-5,6-dihydro-8H-imidazo[2,1-c][1,4]oxazine (168 mg, 0.62 mmol) and Intermediate 13 (285 mg, 0.65 mmol) in 1,4-dioxane (6 mL) and water (1.5 mL) was degassed with nitrogen. Pd-118 (61 mg, 0.093 mmol) was quickly added, the solution degassed again for 10 min and the mixture left to stir at rt for 16 h. The solution was degassed and additional Pd-118 (61 mg, 0.093 mmol) was added, followed by another 10 min of degassing. The mixture was heated at 45° C. for 16 h. The reaction mixture was diluted with EtOAc (20 mL), washed with water (3×20 mL) and brine (20 mL). The organic extract was dried over MgSO₄, filtered and concentrated under reduced pressure to give the crude product, which was purified by automated column chromatography (12 g silica, eluting with 0-10% MeOH/DCM) followed by purification by flash reverse-phase automated chromatography (30 g HPC18 Aq, gradient from water to MeOH). The sample was stirred in ether (5 mL) for 15 min, the solid left to settle and the supernatant removed by pipette. The resulting solid was further dried in a vacuum oven at 40° C. for 2 h to give N-[5-(6,8-dihydro-5H-imidazo[2,1-c][1,4]oxazin-3-yl)-3-methoxy-pyrazin-2-yl]-5-methyl-3-phenyl-isoxazole-4-carboxamide (27 mg, 10%) as a colourless solid. ¹H NMR (500 MHZ, Chloroform-d) δ8.20 (s, 1H), 7.85 (s, 1H), 7.63 (d, J=7.4 Hz, 2H), 7.59 (d, J=6.9 Hz, 1H), 7.55 (t, J=7.3 Hz, 2H), 7.41 (s, 1H), 4.90 (s, 2H), 4.33 (t, J=5.2 Hz, 2H), 4.05 (t, J=5.2 Hz, 2H), 3.69 (s, 3H), 2.83 (s, 3H). m/z 433.1 [M+H]⁺.

### Example 156 N-[5-[2-(Aminomethyl)pyrimidin-5-yl]-3-methoxy-pyrazin-2-yl]-5-methyl-3-phenyl-isoxazole-4-carboxamide hydrochloride

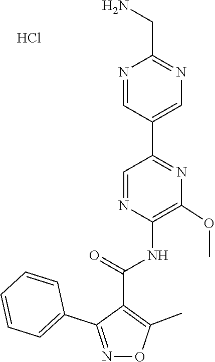 [SMILES: COc1nc(-c2cnc(CN)nc2)cnc1NC(=O)c1c(-c2ccccc2)noc1C ; Cl]

### Step 1

A mixture of Intermediate 13 (220 mg, 0.50 mmol), Intermediate 11 (150 mg, 0.52 mmol) and ground KF (88 mg, 1.5 mmol) in 1,4-dioxane (2.5 mL) and water (1.5 mL) was sparged with nitrogen. Pd-118 (33 mg, 0.050 mmol) was added and mixture heated at 45° C. for 16 h. The solvent was removed under vacuum and the mixture was partitioned between DCM (20 mL) and brine (10 mL). The layers were separated and the aqueous layer was extracted with DCM (2×20 mL). The organic extracts were concentrated to dryness and purified by flash silica column chromatography (12 g silica, elution with 50-100% EtOAc/PE). The isolated crude material was recrystallised from isopropanol to give tert-butyl N-[[5-[6-methoxy-5-[(5-methyl-3-phenyl-isoxazole-4-carbonyl)amino] pyrazin-2-yl]pyrimidin-2-yl]methyl]carbamate (170 mg, 62% yield). ¹H NMR (500 MHZ, Chloroform-d) δ9.18 (s, 2H), 8.39 (s, 1H), 8.01 (s, 1H), 7.67-7.61 (m, 3H), 7.58 (t, J=7.4 Hz, 2H), 5.89-5.37 (m, 1H), 4.65 (d, J=5.2 Hz, 2H), 3.77 (s, 3H), 2.86 (s, 3H), 1.49 (s, 9H). m/z 518.1 [M+H]⁺.

### Step 2

To a suspension of tert-butyl N-[[5-[6-methoxy-5-[(5-methyl-3-phenyl-isoxazole-4-carbonyl)amino] pyrazin-2-yl]pyrimidin-2-yl]methyl]carbamate (180 mg, 0.35 mmol) in 1,4-dioxane (1 mL) was added 2M HCl (in ether, 5 mL, 10 mmol) and the resulting mixture was stirred at room temperature for 16 h. The solid was collected by filtration, washed with ether and dried in the vacuum oven at 40° C. for 6 h to give N-[5-[2-(aminomethyl)pyrimidin-5-yl]-3-methoxy-pyrazin-2-yl]-5-methyl-3-phenyl-isoxazole-4-carboxamide hydrochloride (160 mg, 99% yield). ¹H NMR (500 MHZ, DMSO-d₆) δ10.73 (s, 1H), 9.50 (s, 2H), 8.74 (s, 1H), 8.56 (s, 3H), 7.60 (d, J=7.6 Hz, 2H), 7.48 (t, J=7.3 Hz, 1H), 7.42 (t, J=7.5 Hz, 2H), 4.00 (s, 3H), 2.63 (s, 3H). CH₂ under NMR solvent peak. m/z 418.1 [M+H for free base]⁺.

### Example 157 N-(2-Methoxy-6-(pyrimidin-5-yl)pyridin-3-yl)-4-methyl-1-phenyl-1H-1,2,3-triazole-5-carboxamide

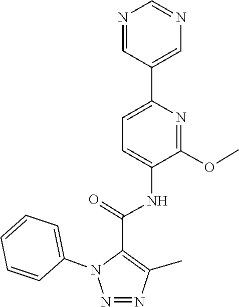 [SMILES: COc1nc(-c2cncnc2)ccc1NC(=O)c1c(C)nnn1-c1ccccc1]

Intermediate 10 (100 mg, 0.26 mmol) was added to a solution of 5-(4,4,5,5-tetramethyl-1,3,2-dioxaborolan-2-yl)pyrimidine (58 mg, 0.28 mmol) and KF (45 mg, 0.77 mmol) in 1,4-dioxane (3 mL) and water (1 mL). The reaction mixture was degassed with nitrogen. Pd-118 (17 mg, 0.030 mmol) was quickly added and the mixture further degassed before stirring at 50° C. for 16 h. The reaction mixture was concentrated under reduced pressure. The dark residue dissolved in DCM (10 mL) and washed with water (2×10 mL). The organic layer was dried over MgSO₄, filtered and concentrated under reduced pressure. The residue was purified by flash column chromatography (4 g silica, eluting with 0-100% EtOAc/PE+1% Et₃N) to afford N-(2-methoxy-6-(pyrimidin-5-yl)pyridin-3-yl)-4-methyl-1-phenyl-1H-1,2,3-triazole-5-carboxamide (28 mg, 27% yield) as a yellow solid. ¹H NMR (500 MHZ, Chloroform-d) δ9.28 (s, 2H), 9.20 (s, 1H), 8.73 (d, J=8.2 Hz, 1H), 7.93 (br s, 1H), 7.54-7.65 (m, 5H), 7.40 (d, J=8.1 Hz, 1H), 3.95 (s, 3H), 2.73 (s, 3H). m/z 388.1 [M+H]⁺.

### Example 158 N-[5-[5-(Difluoromethoxy)-3-pyridyl]-3-methoxy-pyrazin-2-yl]-5-methyl-3-phenyl-isoxazole-4-carboxamide

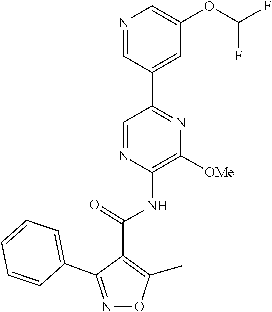 [SMILES: COc1nc(-c2cncc(OC(F)F)c2)cnc1NC(=O)c1c(-c2ccccc2)noc1C]

### Step 1

5-Brompyridin-3-ol (868 mg, 4.99 mmol) and KOH (5.88 g, 105 mmol) were dissolved in water (20 mL) and MeCN (20 mL). The solution was cooled to −78° C. whereupon the mixture solidified. 2-Chloro-2,2-difluoroacetophenone (0.88 mL, 6.0 mmol) was added drop-wise, upon completion of addition the mixture was allowed to warm up first to room temperature and then heated at 80° C. for 4 h. After allowing to cool to room temperature, the reaction mixture was diluted with ether (80 mL), washed with water (6×40 mL), dried over MgSO₄, filtered and concentrated under reduced pressure (with an unheated water bath) to give 3-bromo-5-(difluoromethoxy)pyridine (269 mg, 22% yield) as a colourless liquid, which was used without further purification. The sample was kept in a tin-foil protected flask at low temperature. ¹H NMR (500 MHZ, Chloroform-d) δ8.57 (d, J=2.0 Hz, 1H), 8.44 (d, J=2.4 Hz, 1H), 7.69 (d, J=2.3 Hz, 1H), 6.58 (t, J=72.0 Hz, 1H). ¹⁹F NMR (376 MHZ, Chloroform-d) δ-81.9 (d, J=72 Hz, 2F). m/z 225.9 [M+H, ⁸¹Br]⁺.

### Step 2

A solution of KF (153 mg, 2.63 mmol), Intermediate 13 (380 mg, 0.88 mmol) and 3-bromo-5-(difluoromethoxy)pyridine (269 mg, 1.20 mmol) in 1,4-dioxane (6.5 mL) and water (1.5 mL) was degassed with nitrogen. Pd-118 (57 mg, 0.088 mmol) was added, the solution degassed again and the mixture left to stir at rt for 16 h. The reaction mixture was diluted with EtOAc (20 mL), washed with water (3×20 mL) and brine (20 mL). The organic extract was dried over MgSO₄, filtered and concentrated under reduced pressure. The crude material was purified by automated column chromatography (12 g silica, eluting with 0-10% MeOH/DCM) to afford N-[5-[5-(difluoromethoxy)-3-pyridyl]-3-methoxy-pyrazin-2-yl]-5-methyl-3-phenyl-isoxazole-4-carboxamide (221 mg, 55% yield) as a light brown solid. ¹H NMR (500 MHZ, Chloroform-d) δ8.99 (s, 1H), 8.48 (d, J=2.5 Hz, 1H), 8.40 (s, 1H), 8.01 (s, 1H), 7.95 (s, 1H), 7.62 (m, 3H), 7.56 (t, J=7.7 Hz, 2H), 6.61 (t, J=72.0 Hz, 1H), 3.76 (s, 3H), 2.84 (s, 3H). ¹⁹F NMR (376 MHZ, Chloroform-d) 0-81.3 (d, J=72.0 Hz, 2F). m/z 454.0 [M+H]⁺.

### Example 159 N-(6-(2-Amino-[1,2,4]triazolo[1,5-a]pyridin-7-yl)-2-methoxypyridin-3-yl)-4-methyl-1-phenyl-1H-1,2,3-triazole-5-carboxamide

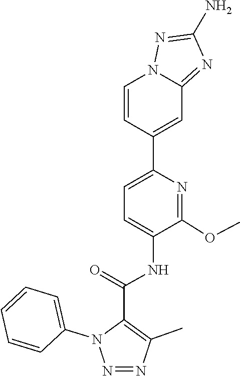 [SMILES: COc1nc(-c2ccn3nc(N)nc3c2)ccc1NC(=O)c1c(C)nnn1-c1ccccc1]

### Step 1—Synthesis of Intermediate 14-N-(2-Methoxy-6-(4,4,5,5-tetramethyl-1,3,2-dioxaborolan-2-yl)pyridin-3-yl)-4-methyl-1-phenyl-1H-1,2,3-triazole-5-carboxamide

Intermediate 10 (936 mg, 2.15 mmol), B₂pin₂ (708 mg, 2.79 mmol), Pd(dppf)Cl₂ (177 mg, 0.21 mmol) and KOAc (632 mg, 6.44 mmol) were dissolved in 1,4-dioxane (15 mL). The reaction mixture was degassed with nitrogen before heated at 90° C. for 1 h. After cooling to rt, the reaction mixture was diluted with EtOAc (50 mL), washed with water (2×50 mL) and the organic layer dried over MgSO₄, filtered and concentrated under reduced pressure. The residue was purified by trituration with PE (50 mL) to afford N-(2-methoxy-6-(4,4,5,5-tetramethyl-1,3,2-dioxaborolan-2-yl)pyridin-3-yl)-4-methyl-1-phenyl-1H-1,2,3-triazole-5-carboxamide (1.03 g, 99% yield) as a brown solid. ¹H NMR (500 MHZ, Chloroform-d) δ8.54 (d, J=7.8 Hz, 1H), 7.97 (br s, 1H), 7.51-7.62 (m, 5H), 7.46 (d, J=7.8 Hz, 1H), 3.91 (s, 3H), 2.70 (s, 3H), 1.35 (s, 12H). m/z 354.0 [M+H for boronic acid]⁺.

### Step 2

Intermediate 14 (100 mg, 0.23 mmol) was added to a solution 7-bromo-[1,2,4]triazolo[1,5-a]pyridin-2-amine (53 mg, 0.25 mmol) and KF (40 mg, 0.69 mmol) in 1,4-dioxane (3 mL) and water (1 mL). The reaction mixture was degassed with nitrogen. Pd-118 (15 mg, 0.02 mmol) was added and the mixture further degassed before stirring at 80° C. for 16 h. The reaction mixture was then concentrated under reduced pressure. The dark residue dissolved in DCM (10 mL) and washed with water (2×10 mL). The organic layer was dried over MgSO₄, filtered and concentrated under reduced pressure. The residue was purified by flash column chromatography (12 g, silica, eluting with 0-10% MeOH/DCM) to afford N-(6-(2-amino-[1,2,4]triazolo[1,5-a]pyridin-7-yl)-2-methoxypyridin-3-yl)-4-methyl-1-phenyl-1H-1,2,3-triazole-5-carboxamide (33 mg, 31% yield) as a cream solid. ¹H NMR (500 MHZ, Chloroform-d) δ8.71 (d, J=8.1 Hz, 1H), 8.31 (d, J=7.1 Hz, 1H), 8.03 (s, 1H), 7.93 (br s, 1H), 7.54-7.66 (m, 5H), 7.46 (d, J=7.0 Hz, 1H), 7.43 (d, J=8.1 Hz, 1H), 4.47 (br s, 2H), 3.96 (s, 3H), 2.73 (s, 3H). m/z 442.0 [M+H]⁺.

### Example 160 N-(6′-(Hydroxymethyl)-6-methoxy-[2,3′-bipyridin]-5-yl)-4-methyl-1-phenyl-1H-1,2,3-triazole-5-carboxamide

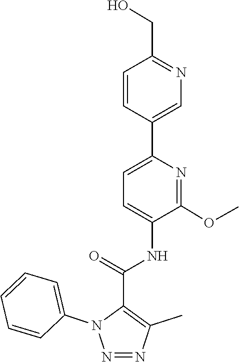 [SMILES: COc1nc(-c2ccc(CO)nc2)ccc1NC(=O)c1c(C)nnn1-c1ccccc1]

Intermediate 14 (100 mg, 0.23 mmol) was added to a solution of (5-bromopyridin-2-yl) methanol (47 mg, 0.25 mmol) and KF (40 mg, 0.69 mmol) in 1,4-dioxane (3 mL) and water (1 mL). The reaction mixture was degassed with nitrogen. Pd-118 (15 mg, 0.02 mmol) was added and the mixture further degassed before stirring at 50° C. for 16 h. The reaction mixture was then concentrated under reduced pressure. The dark residue dissolved in DCM (10 mL) and washed with water (2×10 mL). The organic layer was dried over MgSO₄, filtered and concentrated under reduced pressure. The residue was purified by flash column chromatography (12 g, silica, eluting with 0-10% MeOH/DCM) to afford N-(6′-(hydroxymethyl)-6-methoxy-[2,3′-bipyridin]-5-yl)-4-methyl-1-phenyl-1H-1,2,3-triazole-5-carboxamide (43 mg, 43% yield) as a cream solid. ¹H NMR (500 MHZ, Chloroform-d) δ9.16 (s, 1H), 8.68 (d, J=8.1 Hz, 1H), 8.24 (d, J=8.1 Hz, 1H), 7.91 (br s, 1H), 7.54-7.65 (m, 5H), 7.37 (d, J=8.2 Hz, 1H), 7.31 (d, J=8.2 Hz, 1H), 4.81 (d, J=4.6 Hz, 2H), 3.95 (s, 3H), 3.64 (br t, J=5.1 Hz, 1H), 2.72 (s, 3H). m/z 417.1 [M+H]⁺.

### Example 161 N-(6-(2-((Dimethylamino)methyl) pyrimidin-5-yl)-2-methoxypyridin-3-yl)-4-methyl-1-phenyl-1H-1,2,3-triazole-5-carboxamide

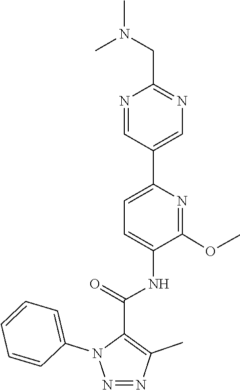 [SMILES: COc1nc(-c2cnc(CN(C)C)nc2)ccc1NC(=O)c1c(C)nnn1-c1ccccc1]

Synthesised according to Example 160 substituting (5-bromopyridin-2-yl) methanol with Intermediate 9 to give N-(6-(2-((dimethylamino)methyl) pyrimidin-5-yl)-2-methoxypyridin-3-yl)-4-methyl-1-phenyl-1H-1,2,3-triazole-5-carboxamide (62 mg, 57% yield) as a brown solid. ¹H NMR (500 MHz, Chloroform-d) δ9.25 (s, 2H), 8.71 (d, J=8.1 Hz, 1H), 7.91 (br s, 1H), 7.53-7.67 (m, 5H), 7.38 (d, J=7.6 Hz, 1H), 3.94 (s, 3H), 3.80 (s, 2H), 2.72 (s, 3H), 2.39 (s, 6H). m/z 445.1 [M+H]⁺.

### Example 162 N-(6-(2-Ethoxypyrimidin-5-yl)-2-methoxypyridin-3-yl)-4-methyl-1-phenyl-1H-1,2,3-triazole-5-carboxamide

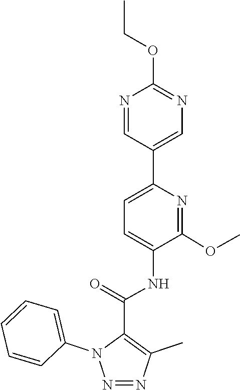 [SMILES: CCOc1ncc(-c2ccc(NC(=O)c3c(C)nnn3-c3ccccc3)c(OC)n2)cn1]

Synthesised according to Example 160 substituting (5-bromopyridin-2-yl) methanol with 5-bromo-2-ethoxypyrimidine to give N-(6-(2-ethoxypyrimidin-5-yl)-2-methoxypyridin-3-yl)-4-methyl-1-phenyl-1H-1,2,3-triazole-5-carboxamide (39 mg, 38% yield) as a yellow solid. ¹H NMR (500 MHZ, Chloroform-d) δ9.05 (s, 2H), 8.67 (d, J=8.2 Hz, 1H), 7.89 (br s, 1H), 7.54-7.65 (m, 5H), 7.27-7.30 (m, 1H), 4.44-4.53 (m, 2H), 3.93 (s, 3H), 2.72 (s, 3H), 1.46 (t, J=7.2 Hz, 3H). m/z 432.1 [M+H]⁺.

### Example 163 N-[6-[2-[2-(Dimethylamino) ethoxy]pyrimidin-5-yl]-2-methoxy-3-pyridyl]-5-methyl-3-phenyl-isoxazole-4-carboxamide

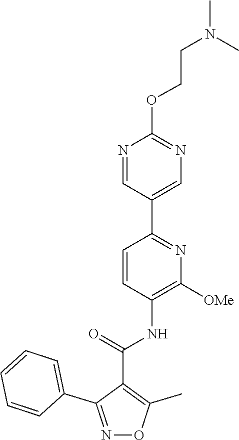 [SMILES: COc1nc(-c2cnc(OCCN(C)C)nc2)ccc1NC(=O)c1c(-c2ccccc2)noc1C]

### Step 1

Sodium metal (190 mg, 8.26 mmol) was added portion-wise to ice-cold 2-dimethylaminoethanol (5.00 mL, 49.7 mmol). The reaction mixture was left to stir until all the sodium has fully dissolved (45 min). 5-Bromo-2-chloropyrimidine (1.00 g, 5.17 mmol) was added (mildly exothermic) and the reaction mixture was left to stir at rt for 15 min before diluted with EtOAc (20 mL), washed with water (7×30 mL) and brine (30 mL). The organic layer was dried over MgSO₄, filtered and concentrated under reduced pressure to give 2-(5-bromopyrimidin-2-yl)oxy-N, N-dimethyl-ethanamine (780 mg, 61% yield) as a colourless liquid, which was used without further purification. ¹H NMR (500 MHZ, Chloroform-d) δ8.50 (s, 2H), 4.43 (t, J=5.9 Hz, 2H), 2.73 (t, J=5.9 Hz, 2H), 2.31 (s, 6H). m/z 247.9 [M+H, ⁸¹Br]⁺.

### Step 2

A solution of KF (76 mg, 1.31 mmol), 2-(5-bromopyrimidin-2-yl)oxy-N,N-dimethyl-ethanamine (110 mg, 0.45 mmol) and Intermediate 7 (200 mg, 0.46 mmol) in 1,4-dioxane (4 mL) and water (1 mL) was degassed with nitrogen. Pd-118 (29 mg, 0.044 mmol) was added and the reaction mixture was further degassed before stirred at rt for 16 h then at 45° C. for 5 h. The reaction mixture was diluted with DCM (50 mL), washed with water (20 mL) and the aqueous phase was extracted with DCM (2×20 mL). The combined organic extracts were dried over MgSO₄, filtered and concentrated under reduced pressure. The crude material was purified by automated column chromatography (12 g silica, eluting with 0-10% MeOH/DCM) to give N-[6-[2-[2-(dimethylamino) ethoxy]pyrimidin-5-yl]-2-methoxy-3-pyridyl]-5-methyl-3-phenyl-isoxazole-4-carboxamide (127 mg, 58% yield) as a beige solid. ¹H NMR (500 MHZ, Chloroform-d) δ9.03 (s, 2H), 8.73 (d, J=8.1 Hz, 1H), 7.75 (s, 1H), 7.67-7.62 (m, 3H), 7.62-7.54 (m, 2H), 7.26 (s, 1H), 4.52 (t, J=6.0 Hz, 2H), 3.68 (s, 3H), 2.84 (s, 3H), 2.79 (t, J=6.0 Hz, 2H), 2.35 (s, 6H). m/z 475.1 [M+H]⁺.

### Example 164 N-(5′-Fluoro-6-methoxy-6′-oxo-1′,6′-dihydro-[2,3′-bipyridin]-5-yl)-4-methyl-1-phenyl-1H-1,2,3-triazole-5-carboxamide

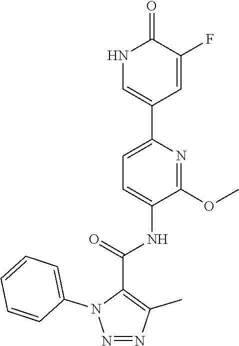 [SMILES: COc1nc(-c2c[nH]c(=O)c(F)c2)ccc1NC(=O)c1c(C)nnn1-c1ccccc1]

Intermediate 14 (100 mg, 0.23 mmol) was added to a solution of 5-bromo-3-fluoropyridin-2 (1H)-one (48 mg, 0.25 mmol) and KF (40 mg, 0.69 mmol) in 1,4-dioxane (3 mL) and water (1 mL). The reaction mixture was degassed with nitrogen. Pd-118 (15 mg, 0.02 mmol) was added and the mixture was further degassed before stirring at 35° C. for 16 h. The reaction mixture was concentrated under reduced pressure and the dark residue was dissolved in DCM (10 mL) and washed with water (2×10 mL). The organic layer was dried over MgSO₄, filtered and concentrated under reduced pressure. The residue was purified by flash column chromatography (12 g, silica, eluting with 0-10% MeOH/DCM) to afford N-(5′-fluoro-6-methoxy-6′-oxo-1′,6′-dihydro-[2,3′-bipyridin]-5-yl)-4-methyl-1-phenyl-1H-1,2,3-triazole-5-carboxamide (28 mg, 28% yield) as a bronze solid. ¹H NMR (500 MHZ, Chloroform-d) δ12.99 (br s, 1H), 8.64 (d, J=8.1 Hz, 1H), 7.99 (br s, 1H), 7.83-7.92 (m, 2H), 7.53-7.65 (m, 5H), 7.08 (d, J=8.1 Hz, 1H), 3.90 (s, 3H), 2.71 (s, 3H). m/z 421.1 [M+H]⁺.

### Example 165 5-(6-Methoxy-5-(4-methyl-1-phenyl-1H-1,2,3-triazole-5-carboxamido)pyridin-2-yl)pyrimidine-2-carboxamide

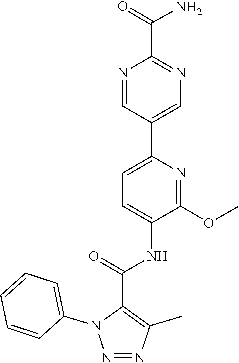 [SMILES: COc1nc(-c2cnc(C(N)=O)nc2)ccc1NC(=O)c1c(C)nnn1-c1ccccc1]

### Step 1

Intermediate 14 (200 mg, 0.46 mmol) was added to a solution of methyl 5-bromopyrimidine-2-carboxylate (110 mg, 0.51 mmol) and KF (80 mg, 1.4 mmol) in 1,4-dioxane (3 mL) and water (1 mL). The reaction mixture was degassed with nitrogen. Pd-118 (30 mg, 0.05 mmol) was added and the mixture further degassed before stirring at 50° C. for 16 h. The reaction mixture was concentrated under reduced pressure and the dark residue was dissolved in DCM (10 mL) and washed with water (2×10 mL). The organic layer was dried over MgSO₄, filtered and concentrated under reduced pressure. The residue was purified by flash column chromatography (12 g, silica, eluting with 0-10% MeOH/DCM) to afford methyl 5-(6-methoxy-5-(4-methyl-1-phenyl-1H-1,2,3-triazole-5-carboxamido)pyridin-2-yl)pyrimidine-2-carboxylate (155 mg, 72% yield) as an orange solid. ¹H NMR (500 MHZ, Chloroform-d) δ9.43 (s, 2H), 8.77 (d, J=8.1 Hz, 1H), 7.95 (br s, 1H), 7.54-7.67 (m, 5H), 7.48 (d, J=8.1 Hz 1H), 4.10 (s, 3H), 3.95 (s, 3H), 2.73 (s, 3H). m/z 446.0 [M+H]⁺.

### Step 2

LiOH (38 mg, 0.51 mmol) was added to a solution of methyl 5-(6-methoxy-5-(4-methyl-1-phenyl-1H-1,2,3-triazole-5-carboxamido)pyridin-2-yl)pyrimidine-2-carboxylate (150 mg, 0.34 mmol) in THF (3 mL) and water (1 mL) and the reaction mixture was stirred at rt for 2 h.

The reaction mixture was concentrated under reduced pressure. 0.1 M HCl (10 mL) was added and the resulting precipitate was filtered, washed with water (10 mL) and EtOAc (10 mL) and then dried under vacuum at 40° C. to afford 5-(6-methoxy-5-(4-methyl-1-phenyl-1H-1,2,3-triazole-5-carboxamido)pyridin-2-yl)pyrimidine-2-carboxylic acid (145 mg, 95% yield) as a cream solid. ¹H NMR (500 MHZ, DMSO-d₆) δ13.62 (br s, 1H), 10.28 (s, 1H), 9.56 (s, 2H), 8.38 (br s, 1H), 7.87 (d, J=8.1 Hz, 1H), 7.50-7.69 (m, 5H), 4.02 (s, 3H). CH₃ under NMR solvent peak. m/z 432.0 [M+H]⁺.

### Step 3

NH₃ (7 M in MeOH, 0.10 mL, 0.70 mmol) was added to a solution of 5-(6-methoxy-5-(4-methyl-1-phenyl-1H-1,2,3-triazole-5-carboxamido)pyridin-2-yl)pyrimidine-2-carboxylic acid (75 mg, 0.17 mmol), HATU (198 mg, 0.52 mmol) and DIPEA (0.12 mL, 0.70 mmol) in DMF (3 mL). The reaction mixture was stirred at rt for 24 h. The resulting precipitate was collected by vacuum filtration, washed with water (10 mL) and ether (3×10 mL), and then dried under vacuum at 40° C. to afford 5-(6-methoxy-5-(4-methyl-1-phenyl-1H-1,2,3-triazole-5-carboxamido)pyridin-2-yl)pyrimidine-2-carboxamide (62 mg, 79% yield) as a colourless solid. ¹H NMR (500 MHZ, DMSO-d₆) δ10.28 (s, 1H), 9.55 (s, 2H), 8.38 (br s, 1H), 8.23 (s, 1H), 7.78-7.98 (m, 2H), 7.50-7.75 (m, 5H), 4.03 (s, 3H). CH₃ under NMR solvent peak. m/z 431.1 [M+H]⁺.

### Example 166 N-(2-Methoxy-6-(2-(methylamino)pyrimidin-5-yl)pyridin-3-yl)-4-methyl-1-phenyl-1H-1,2,3-triazole-5-carboxamide

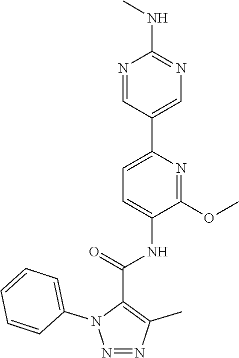 [SMILES: CNc1ncc(-c2ccc(NC(=O)c3c(C)nnn3-c3ccccc3)c(OC)n2)cn1]

Synthesised according to Example 160 substituting (5-bromopyridin-2-yl) methanol with 5-bromo-N-methyl-pyrimidin-2-amine to give N-(2-methoxy-6-(2-(methylamino)pyrimidin-5-yl)pyridin-3-yl)-4-methyl-1-phenyl-1H-1,2,3-triazole-5-carboxamide (30 mg, 30% yield) as a cream solid. ¹H NMR (500 MHZ, Chloroform-d) δ8.89 (s, 2H), 8.61 (d, J=8.1 Hz, 1H), 7.85 (br s, 1H), 7.52-7.65 (m, 5H), 7.18 (d, J=8.1 Hz, 1H), 5.21-5.28 (m, 1H), 3.90 (s, 3H), 3.07 (d, J=4.6 Hz, 3H), 2.71 (s, 3H). m/z 417.1 [M+H]⁺.

### Example 167 N-[6-[2-[2-(Dimethylamino)ethylamino]pyrimidin-5-yl]-2-methoxy-3-pyridyl]-5-methyl-3-phenyl-isoxazole-4-carboxamide

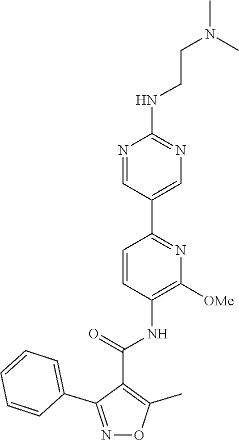 [SMILES: COc1nc(-c2cnc(NCCN(C)C)nc2)ccc1NC(=O)c1c(-c2ccccc2)noc1C]

### Step 1

To a dry 20 mL Biotage microwave vial was added 5-bromo-2-chloropyrimidine (500 mg, 2.6 mmol). The vial was purged with nitrogen for 10 min before EtOH (2.5 mL) and DIPEA (0.28 mL, 2.6 mmol) were added. The vial was sealed and the mixture heated at 80° C. for 16 h. The reaction mixture was diluted with DCM (40 mL), washed with saturated NaHCO₃ (20 mL). The aqueous phase was extracted with DCM (4×20 mL) and the combined the organic extract was dried over MgSO₄, filtered and concentrated under reduced pressure. The crude material was purified by automated column chromatography (12 g silica, eluting with 0-10% MeOH/DCM) to afford N-(5-bromopyrimidin-2-yl)-N′,N′-dimethyl-ethane-1,2-diamine (292 mg, 46% yield) as a light yellow oil, which solidified upon standing. ¹H NMR (500 MHZ, Chloroform-d) δ8.26 (s, 2H), 5.74 (s, 1H), 3.45-3.38 (m, 2H), 2.51 (t, J=6.1 Hz, 2H), 2.25 (s, 6H). m/z 246.9 [M+H, ⁸¹Br]⁺.

### Step 2

A solution of KF (76 mg, 1.3 mmol), N-(5-bromopyrimidin-2-yl)-N′,N′-dimethyl-ethane-1,2-diamine (113 mg, 0.46 mmol) and Intermediate 7 (200 mg, 0.46 mmol) in 1,4-dioxane (4 mL) and water (1 mL) was degassed with nitrogen. Pd-118 (29 mg, 0.044 mmol) was added, the reaction mixture was further degassed and stirred at rt for 16 h. The reaction mixture was diluted with EtOAc (20 mL) and washed with water (3×20 mL). The first aqueous phase was back-extracted with DCM (2×20 mL) and the combined organic extracts were dried over MgSO₄, filtered and concentrated under reduced pressure. The crude material was purified by automated column chromatography (12 g silica, eluting with 0-10% MeOH/DCM) to give crude product which was triturated with MeCN (25 mL) and filtered through filter paper (gravity filtration). The solid was washed with additional MeCN (30 mL), before being redissolved in DCM (40 mL) and the DCM filtrate concentrated under reduced pressure to give N-[6-[2-[2-(dimethylamino)ethylamino]pyrimidin-5-yl]-2-methoxy-3-pyridyl]-5-methyl-3-phenyl-isoxazole-4-carboxamide (47 mg, 22% yield) as a colourless solid. ¹H NMR (500 MHz, Chloroform-d) δ8.84 (s, 2H), 8.65 (d, J=8.0 Hz, 1H), 7.70 (s, 1H), 7.65-7.55 (m, 5H), 7.14 (d, J=8.1 Hz, 1H), 5.90-5.74 (m, 1H), 3.66 (s, 3H), 3.57-3.45 (m, 2H), 2.82 (s, 3H), 2.53 (t, J=6.1 Hz, 2H), 2.26 (s, 6H). m/z 474.1 [M+H]⁺.

### Example 168 N-[2-Methoxy-6-[2-(methylaminomethyl)pyrimidin-5-yl]-3-pyridyl]-5-methyl-3-phenyl-isoxazole-4-carboxamide hydrochloride

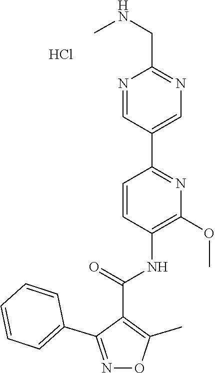 [SMILES: CNCc1ncc(-c2ccc(NC(=O)c3c(-c4ccccc4)noc3C)c(OC)n2)cn1 ; Cl]

### Step 1—Synthesis of Intermediate 15-tert-Butyl N-[(5-bromopyrimidin-2-yl)methyl]-N-methyl-carbamate

To a solution of Intermediate 8 (450 mg, 1.79 mmol) in THF (2.5 mL) was added a 2 M solution of methylamine (2.7 mL, 5.4 mmol) in THF. A white precipitate formed immediately. After 2 h, the suspension was concentrated to dryness and re-suspended in DCM (7.5 mL). Et₃N (0.50 mL, 3.57 mmol) was added and stirred for 10 min before addition of di-tert-butyl dicarbonate (468 mg, 2.14 mmol) and a yellow solution immediately developed. After 1 h, the solution was diluted with water (5 mL) and brine (5 mL). The layers were separated and the organic layer was concentrated to a yellow oil/suspension. The material was purified by flash silica column chromatography (12 g silica, eluting with 0-20% EtOAc/PE) to give tert-butyl N-[(5-bromopyrimidin-2-yl)methyl]-N-methyl-carbamate (300 mg, 53% yield) as a yellow oil. ¹H NMR (500 MHZ, Chloroform-d) δ8.74 (s, 2H), 4.78-4.41 (m, 2H), 3.06-2.94 (m, 3H), 1.54-1.29 (m, 9H). m/z 201.9 [M-Boc+H, ⁷⁹Br]⁺, 203.9 [M-Boc+H, ⁸¹Br]⁺.

### Step 2

A mixture of Intermediate 7 (200 mg, 0.46 mmol), Intermediate 15 (150 mg, 0.50 mmol) and ground KF (80 mg, 1.4 mmol) in 1,4-dioxane (2 mL) and water (1 mL) was sparged with nitrogen. Pd-118 (30 mg, 0.05 mmol) was added and the reaction mixture was heated at 50° C. for 16 h. The reaction mixture was partitioned between DCM (10 mL) and brine (5 mL), and separated. The aqueous layer was extracted DCM (2×10 mL) and the combined organic extracts were concentrated to dryness. The crude material was purified by flash silica column chromatography (12 g silica, eluting with 60-100% EtOAc/PE) to afford the Boc-protected product as a yellow solid. The solid was suspended in 4 M HCl (4 mL, 16 mmol) in 1,4-dioxane and stirred for 1 h. The suspension was diluted with TBME (40 mL) and the solid was collected by vacuum filtration, washed with TBME (2×40 mL) and dried in a vacuum oven to afford N-[2-methoxy-6-[2-(methylaminomethyl)pyrimidin-5-yl]-3-pyridyl]-5-methyl-3-phenyl-isoxazole-4-carboxamide hydrochloride (160 mg, 69% yield) as a light yellow solid. ¹H NMR (500 MHZ, DMSO-d₆) δ9.56 (s, 1H), 9.49 (s, 2H), 9.32 (s, 2H), 8.45 (s, 1H), 7.84 (d, J=8.1 Hz, 1H), 7.71 (d, J=7.0 Hz, 2H), 7.61-7.51 (m, 3H), 4.50 (t, J=6.2 Hz, 2H), 2.72 (d, J=5.7 Hz, 3H), 2.67 (s, 3H). Note CH₃ under water peak. m/z 431.1 [M+H for free base]⁺.

### Example 169 N-[3-Methoxy-5-[2-(methylaminomethyl)pyrimidin-5-yl] pyrazin-2-yl]-5-methyl-3-phenyl-isoxazole-4-carboxamide hydrochloride

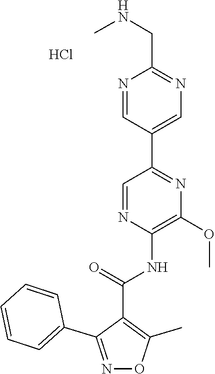 [SMILES: CNCc1ncc(-c2cnc(NC(=O)c3c(-c4ccccc4)noc3C)c(OC)n2)cn1 ; Cl]

A mixture of Intermediate 13 (160 mg, 0.37 mmol), Intermediate 15 (150 mg, 0.50 mmol) and ground KF (80 mg, 1.4 mmol) in 1,4-dioxane (1.5 mL) and water (0.8 mL) was sparged with nitrogen. Pd-118 (30 mg, 0.05 mmol) was added and the reaction mixture was heated at 50° C. for 16 h. The reaction mixture was partitioned between DCM (10 mL) and brine (5 mL), and separated. The aqueous layer was extracted with DCM (2×10 mL). The combined organic extracts were concentrated to dryness and purified by flash silica column chromatography (12 g silica, eluting with 40-100% EtOAc/PE) to give the Boc-protected product as a yellow foam. The material was stirred in 4 M HCl (4.0 mL, 16 mmol) in 1,4-dioxane for 1 h at rt and diluted with TBME (40 mL) and the solids were collected by vacuum filtration, washed with TBME (2×40 mL) and dried o/n in a vacuum oven at 40° C. to afford N-[3-methoxy-5-[2-(methylaminomethyl)pyrimidin-5-yl] pyrazin-2-yl]-5-methyl-3-phenyl-isoxazole-4-carboxamide hydrochloride (120 mg, 66% yield) as an off-white solid. ¹H NMR (500 MHZ, DMSO-d₆) δ10.73 (s, 1H), 9.51 (s, 2H), 9.28 (s, 2H), 8.74 (s, 1H), 7.61-7.57 (m, 2H), 7.52-7.45 (m, 1H), 7.45-7.40 (m, 2H), 4.57-4.51 (m, 2H), 4.00 (s, 3H), 2.73 (s, 3H), 2.63 (s, 3H). m/z 432.1 [M+H for free base]⁺.

### Example 170 N-[2-Methoxy-6-[2-[2-(methylamino)ethylamino]pyrimidin-5-yl]-3-pyridyl]-5-methyl-3-phenyl-isoxazole-4-carboxamide hydrochloride

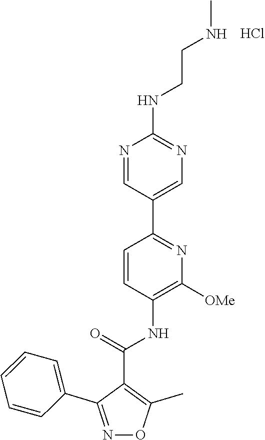 [SMILES: CNCCNc1ncc(-c2ccc(NC(=O)c3c(-c4ccccc4)noc3C)c(OC)n2)cn1 ; Cl]

### Step 1

To a dry 20 mL Biotage microwave vial was added 5-bromo-2-chloropyrimidine (0.25 g, 1.3 mmol), DIPEA (0.27 mL, 1.6 mmol), tert-butyl (2-aminoethyl)methylcarbamate (225 mg, 1.29 mmol), and EtOH (1.25 mL). The vial was capped and the mixture was heated at 70° C. for 16 h. The reaction mixture was diluted with EtOAc (20 mL), washed with water (3×20 mL) and brine (20 mL). The organic layer was dried over MgSO₄, filtered and concentrated under reduced pressure to give the crude product, which was purified by automated column chromatography (12 g silica, eluting with 0-100% EtOAc/PE) to afford tert-butyl N-[2-[(5-bromopyrimidin-2-yl)amino]ethyl]-N-methyl-carbamate (401 mg, 94% yield) as a colourless oil. ¹H NMR (500 MHz, Chloroform-d) δ 8.26 (s, 2H), 5.64 (s, 0.5H), 5.35 (s, 0.5H), 3.57-3.48 (m, 2H), 3.49-3.38 (m, 2H), 2.88 (s, 3H), 1.43 (s, 9H). m/z 333.0 [M+H, ⁸¹Br]⁺.

### Step 2

A solution of KF (76 mg, 1.3 mmol), tert-butyl N-[2-[(5-bromopyrimidin-2-yl)amino]ethyl]-N-methyl-carbamate (152 mg, 0.46 mmol) and Intermediate 7 (200 mg, 0.46 mmol) in 1,4-dioxane (4 mL) and water (1 mL) was degassed with nitrogen. Pd-118 (29 mg, 0.044 mmol) was added and the reaction mixture was degassed again and left to stir at rt for 16 h, then heated at 45° C. for 5 h. The reaction mixture was diluted with DCM (50 mL), washed with water (20 mL) and the aqueous phase back-extracted with additional DCM (2×20 mL). The combined organic extracts were dried over MgSO₄, filtered and concentrated under reduced pressure. The crude material was purified by flash column chromatography (12 g silica, eluting with 10% MeOH/DCM), then the crude product was purified again by flash column chromatography (12 g silica, eluting with 0-100% EtOAc/PE). The isolated residue was triturated with MeOH (70 mL), the suspension filtered and the filtrate was concentrated under reduced pressure to afford the Boc-protected product (20 mg). The material was dissolved in DCM (1 mL) and was treated with 4 M HCl in 1,4-dioxane (1 mL, 4 mmol). The mixture was left to stir at rt for 2 h. The sample was concentrated under reduced pressure to give N-[2-methoxy-6-[2-[2-(methylamino)ethylamino]pyrimidin-5-yl]-3-pyridyl]-5-methyl-3-phenyl-isoxazole-4-carboxamide hydrochloride (20 mg, 9% yield) as a beige solid. ¹H NMR (500 MHz, DMSO-d₆) δ9.48 (s, 1H), 9.00 (s, 2H), 8.66 (s, 2H), 8.28 (d, J=8.1 Hz, 1H), 7.77-7.69 (m, 2H), 7.65 (t, J=5.7 Hz, 1H), 7.61-7.50 (m, 4H), 3.89 (s, 3H), 3.67-3.59 (m, 2H), 3.17-3.07 (m, 2H), 2.66 (s, 3H), 2.59 (t, J=5.4 Hz, 3H). m/z 460.1 [M+H]⁺.

### Example 171 N-[6-[2-(2-Aminoethylamino)pyrimidin-5-yl]-2-methoxy-3-pyridyl]-5-methyl-3-phenyl-isoxazole-4-carboxamide hydrochloride

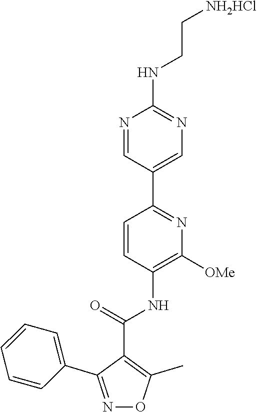

### Step 1

To a dry 20 mL Biotage microwave vial was added 5-bromo-2-chloropyrimidine (1.00 g, 5.17 mmol), EtOH (5 mL) and ethylenediamine (0.39 mL, 5.2 mmol). The vial was sealed and the mixture heated at 80° C. for 16 h. The reaction mixture was concentrated under reduced pressure and then partitioned between EtOAc (30 mL) and water (20 mL). The layers were separated and the aqueous phase was extracted with DCM (3×30 mL). The combined organic extracts were dried over MgSO₄, filtered and concentrated under reduced pressure to give N′-(5-bromopyrimidin-2-yl) ethane-1,2-diamine (600 mg, 51% yield) as a colourless solid, which was used without further purification. ¹H NMR (500 MHZ, Chloroform-d) δ8.27 (s, 2H), 5.59 (s, 1H), 3.44 (q, J=6.0 Hz, 2H), 2.91 (t, J=6.1 Hz, 2H). m/z 219.0 [M+H, ⁸¹Br]⁺.

### Step 2

To a suspension of N′-(5-bromopyrimidin-2-yl) ethane-1,2-diamine (250 mg, 1.15 mmol) and DIPEA (0.23 mL, 1.3 mmol) in DCM (10 mL) was added a solution of di-tert-butyl dicarbonate (287 mg, 1.31 mmol) in DCM (5 mL) and DMAP (14 mg, 0.11 mmol). The reaction mixture was left to stir at rt for 5 min. The reaction mixture was diluted with EtOAc (20 mL), washed with water (3×20 mL) and brine (20 mL). The organic extract was dried over MgSO₄, filtered and concentrated under reduced pressure to give the crude product, which was purified by automated column chromatography (12 g silica, eluting with 0-50% EtOAc/PE) to give tert-butyl N-[2-[(5-bromopyrimidin-2-yl)amino]ethyl]carbamate (178 mg, 49% yield) as a colourless solid. ¹H NMR (500 MHZ, Chloroform-d) δ8.26 (s, 2H), 5.69-5.56 (m, 1H), 5.00-4.81 (m, 1H), 3.54-3.46 (m, 2H), 3.39-3.29 (m, 2H), 1.43 (s, 9H). m/z 319.0 [M+H, ⁸¹Br]⁺.

### Step 3

A solution of KF (76 mg, 1.3 mmol), tert-butyl N-[2-[(5-bromopyrimidin-2-yl)amino]ethyl]carbamate (146 mg, 0.46 mmol) and Intermediate 7 (200 mg, 0.46 mmol) in 1,4-dioxane (4 mL) and water (1 mL) was degassed with nitrogen. Pd-118 (29 mg, 0.044 mmol) was added and the reaction mixture was degassed again and left to stir at rt for 16 h, then at 45° C. for 5 h. The solution was diluted with DCM (50 mL), washed with water (20 mL) and the aqueous phase back-extracted with DCM (2×20 mL). The combined organic extracts were dried over MgSO₄, filtered and concentrated under reduced pressure to give the crude product, which was purified by automated column chromatography (12 g silica, eluting with 0-10% MeOH/DCM) to afford the Boc-protected product as a colourless solid. This was suspended in DCM (10 mL) and then treated with 4 M HCl in 1,4-dioxane (2.5 mL, mmol). The mixture was stirred at rt for 2 h and the volatiles were removed under reduced pressure to give N-[6-[2-(2-aminoethylamino)pyrimidin-5-yl]-2-methoxy-3-pyridyl]-5-methyl-3-phenyl-isoxazole-4-carboxamide hydrochloride (110 mg, 50% yield) as a light yellow solid. ¹H NMR (500 MHZ, DMSO-d₆) δ9.48 (s, 1H), 9.05 (s, 2H), 8.29 (d, J=7.9 Hz, 1H), 8.25-8.15 (m, 3H), 7.96 (s, 1H), 7.75-7.68 (m, 2H), 7.59-7.1 (m, 4H), 3.89 (s, 3H), 3.65 (t, J=6.2 Hz, 2H), 3.03 (q, J=6.0 Hz, 2H), 2.66 (s, 3H). m/z 446.1 [M+H for free base]⁺.

### Example 172 N-[6-[2-[2-(Dimethylamino)ethylcarbamoyl]pyrimidin-5-yl]-2-methoxy-3-pyridyl]-5-methyl-3-phenyl-isoxazole-4-carboxamide

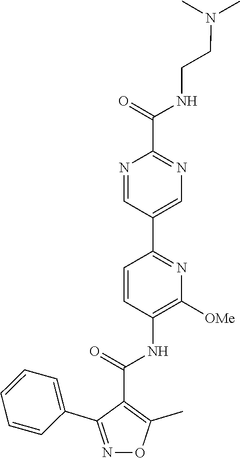 [SMILES: COc1nc(-c2cnc(C(=O)NCCN(C)C)nc2)ccc1NC(=O)c1c(-c2ccccc2)noc1C]

### Step 1

5-Bromo-2-pyrimidinecarboxylic acid (300 mg, 1.48 mmol) and SOCl₂ (2.00 mL, 27.4 mmol) were heated in a sealed microwave vial for 16 h at 65° C. The mixture was left to cool to room temperature and chloroform (2 mL) was added followed by the drop-wise addition of DMF (3 drops). The mixture was then reheated at 80° C. for 16 h whereupon all material dissolved. Once left to cool to rt, the volatiles were removed under reduced pressure. The acid chloride was dissolved in DCM (2.5 mL) and was added drop-wise to a stirring solution of N′,N′-dimethylethane-1,2-diamine (130 mg, 1.48 mmol) and DIPEA (0.31 mL, 1.8 mmol) in DCM (2.5 mL) and the reaction mixture was left to stir for 15 min. The reaction mixture was diluted with DCM (10 mL) and washed with 2 M NaOH (30 mL). The aqueous phase was twice back-extracted with DCM (2×15 mL), the combined organic extracts were dried over MgSO₄, filtered and concentrated under reduced pressure to give the crude product, which was purified by automated column chromatography on silica (12 g silica, eluting with 0-10% MeOH/DCM) to give 5-bromo-N-[2-(dimethylamino)ethyl]pyrimidine-2-carboxamide (186 mg, 44% yield) as an orange-brown solid. ¹H NMR (500 MHz, Chloroform-d) δ8.84 (s, 2H), 8.27-8.12 (m, 1H), 3.54-3.46 (m, 2H), 2.46 (t, J=6.0 Hz, 2H), 2.20 (s, 6H). m/z 275.0 [M+H, ⁸¹Br]⁺.

### Step 2

A solution of KF (76 mg, 1.3 mmol), 5-bromo-N-[2-(dimethylamino)ethyl]pyrimidine-2-carboxamide (126 mg, 0.46 mmol) and Intermediate 7 (200 mg, 0.46 mmol) in 1,4-dioxane (4 mL) and water (1 mL) was degassed with nitrogen. Pd-118 (29 mg, 0.044 mmol) was added and the reaction mixture was degassed again and then left to stir at rt for 48 h. The reaction mixture was diluted with DCM (50 mL), washed with water (20 mL) and the aqueous phase back-extracted with additional DCM (2×20 mL). The combined organic extract was dried over MgSO₄, filtered and concentrated under reduced pressure to give the crude product, which was purified by automated column chromatography (12 g silica, eluting with 0-10% MeOH/DCM) to give N-[6-[2-[2-(dimethylamino)ethylcarbamoyl]pyrimidin-5-yl]-2-methoxy-3-pyridyl]-5-methyl-3-phenyl-isoxazole-4-carboxamide (82 mg, 36% yield) as a beige solid. ¹H NMR (500 MHZ, DMSO-d₆) δ9.54 (s, 2H), 9.52 (s, 1H), 8.76 (t, J=5.7 Hz, 1H), 8.48 (d, J=7.7 Hz, 1H), 7.87 (d, J=8.1 Hz, 1H), 7.74-7.68 (m, 2H), 7.60-7.52 (m, 3H), 3.94 (s, 3H), 3.46-3.39 (m, 2H), 2.67 (s, 3H), 2.46 (t, J=6.5 Hz, 2H), 2.21 (s, 6H). m/z 502.1 [M+H]⁺.

### Example 173 N-[2-Methoxy-6-[2-[2-(methylamino)ethylcarbamoyl]pyrimidin-5-yl]-3-pyridyl]-5-methyl-3-phenyl-isoxazole-4-carboxamide hydrochloride

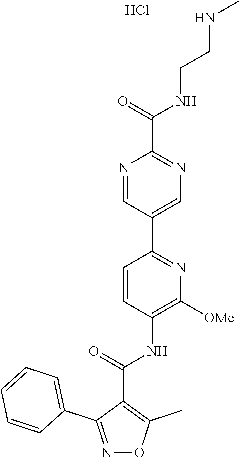 [SMILES: CNCCNC(=O)c1ncc(-c2ccc(NC(=O)c3c(-c4ccccc4)noc3C)c(OC)n2)cn1 ; Cl]

### Step 1

5-Bromo-2-pyrimidinecarboxylic acid (300 mg, 1.48 mmol), SOCl₂ (2.0 mL, 27 mmol), chloroform (2 mL) and DMF (3 drops) were heated at 80° C. for 16 h. After allowing to cool to rt, the volatiles were removed under reduced pressure to afford crude acid chloride. The acid chloride was redissolved in DCM (2.5 mL) and added drop-wise to a stirring solution of DIPEA (0.31 mL, 1.77 mmol) and tert-butyl N-(2-aminoethyl)-N-methyl-carbamate (258 mg, 1.48 mmol) in DCM (2.5 ml). The reaction mixture was left to stir at rt for 20 min. Water (10 mL) was added followed by DCM (20 mL). The layers were separated, the aqueous layer twice extracted with DCM (2×15 mL). The combined organic extracts were washed with 2 M NaOH solution (2×20 mL), dried over MgSO₄, filtered and concentrated under reduced pressure to give the crude product, which was purified by automated column chromatography on silica (12 g silica, eluting with 0-10% MeOH/DCM) to give tert-butyl N-[2-[(5-bromopyrimidine-2-carbonyl)amino]ethyl]-N-methyl-carbamate (382 mg, 68% yield) as a light yellow oil, which solidified upon standing. ¹H NMR (500 MHZ, Chloroform-d) δ8.80 (s, 2H), 8.34 (s, 0.5H), 8.03 (s, 0.5H), 3.62-3.49 (m, 2H), 3.43 (t, J=5.9 Hz, 2H), 2.80 (s, 3H), 1.40-1.26 (m, 9H). m/z 261.0 [M-Boc+H, ⁸¹Br]⁺.

### Step 2

A solution of KF (76 mg, 1.3 mmol), tert-butyl N-[2-[(5-bromopyrimidine-2-carbonyl)amino]ethyl]-N-methyl-carbamate (165 mg, 0.46 mmol) and Intermediate 7 (200 mg, 0.46 mmol) in 1,4-dioxane (4 mL) and water (1 mL) was degassed with nitrogen. Pd-118 (28 mg, 0.044 mmol) was added and the reaction mixture was further degassed and left to stir at rt for 16 h, then heated at 45° C. for 5 h. The reaction mixture was diluted with DCM (50 mL), washed with water (20 mL) and the aqueous phase back-extracted with additional DCM (2×20 mL). The combined organic extracts were dried over MgSO₄, filtered and concentrated under reduced pressure to give the crude product, which was purified by automated column chromatography (12 g silica, eluting with 0-10% MeOH/DCM) to give the Boc-protected product. The material was dissolved in DCM (6.8 mL) and was treated with 4 M HCl in 1,4-dioxane (2.5 mL) and the reaction mixture was left to stir at rt for 2 h. The volatiles were removed under reduced pressure to give N-[2-methoxy-6-[2-[2-(methylamino)ethylcarbamoyl]pyrimidin-5-yl]-3-pyridyl]-5-methyl-3-phenyl-isoxazole-4-carboxamide hydrochloride (145 mg, 96% yield) as a yellow solid. ¹H NMR (500 MHZ, DMSO-d₆) δ9.60-9.51 (m, 3H), 9.22 (t, J=6.0 Hz, 1H), 8.87-8.76 (m, 2H), 8.48 (s, 1H), 7.91 (d, J=8.1 Hz, 1H), 7.76-7.65 (m, 2H), 7.61-7.51 (m, 3H), 3.94 (s, 3H), 3.64 (q, J=6.0 Hz, 2H), 3.16-3.09 (m, 2H), 2.68 (s, 3H), 2.58 (t, J=5.4 Hz, 3H). m/z 488.1 [M+H]⁺.

### Example 174 N-(6-(2-Amino-[1,2,4]triazolo[1,5-a]pyridin-7-yl)-2-methoxypyridin-3-yl)-1-methyl-4-phenyl-1H-1,2,3-5-carboxamide

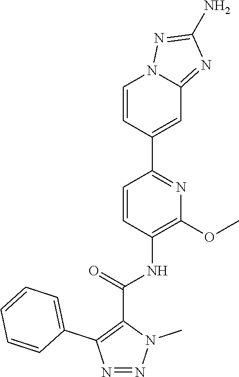 [SMILES: COc1nc(-c2ccn3nc(N)nc3c2)ccc1NC(=O)c1c(-c2ccccc2)nnn1C]

N-(2-Methoxy-6-(4,4,5,5-tetramethyl-1,3,2-dioxaborolan-2-yl)pyridin-3-yl)-1-methyl-4-phenyl-1H-1,2,3-triazole-5-carboxamide (45 mg, 0.10 mmol), synthesised according to Example 154, was added to a solution of 7-bromo[1,2,4]triazolo[1,5-a]pyridin-2-amine (24 mg, 0.11 mmol) and KF (18 mg, 0.31 mmol) in 1,4-dioxane (3 mL) and water (1 mL). The reaction mixture was degassed with nitrogen. Pd-118 (6.7 mg, 0.010 mmol) was added and the reaction mixture was further degassed before stirring at 80° C. for 16 h. The reaction mixture was then concentrated under reduced pressure and the dark residue dissolved in DCM (10 mL) and washed with water (2×10 mL). The organic layer was dried over MgSO₄, filtered and concentrated under reduced pressure. The residue was purified by flash column chromatography (4 g silica, eluting with 0-10% MeOH/DCM) to afford a residue, which was then triturated with MeOH and the precipitate was dried under vacuum at 40° C. to afford N-(6-(2-amino-[1,2,4]triazolo[1,5-a]pyridin-7-yl)-2-methoxypyridin-3-yl)-1-methyl-4-phenyl-1H-1,2,3-triazole-5-carboxamide (12 mg, 26% yield) as a cream solid. ¹H NMR (500 MHZ, Chloroform-d) δ8.75 (d, J=8.1 Hz, 1H), 8.31 (d, J=7.1 Hz, 1H), 8.23 (br s, 1H), 8.03 (s, 1H), 7.64-7.69 (m, 2H), 7.54-7.61 (m, 3H), 7.41-7.49 (m, 2H), 4.47 (br s, 2H), 4.45 (s, 3H), 3.73 (s, 3H). m/z 442.0 [M+H]⁺.

### Example 175 N-[6-[2-(2-Hydroxyethylamino)pyrimidin-5-yl]-2-methoxy-3-pyridyl]-5-methyl-3-phenyl-isoxazole-4-carboxamide

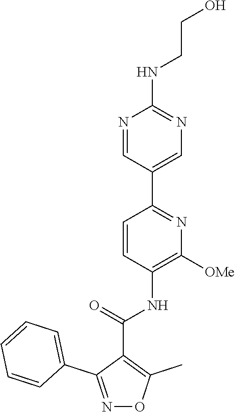 [SMILES: COc1nc(-c2cnc(NCCO)nc2)ccc1NC(=O)c1c(-c2ccccc2)noc1C]

### Step 1

To a 20 mL microwave vial was added 5-bromo-2-chloropyrimidine (500 mg, 2.58 mmol), DIPEA (0.54 mL, 3.1 mmol), ethanolamine (0.17 mL, 2.8 mmol) and EtOH (2.5 mL). The vial was capped and the mixture heated at 80° C. for 2 h. The reaction mixture was partitioned between DCM (30 mL) and water (20 mL). 2 M NaOH (20 mL) was added and the layers separated. The aqueous phase was back-extracted with DCM (2×20 mL), the combined organics dried over MgSO₄, filtered and concentrated under reduced pressure to give 2-[(5-bromopyrimidin-2-yl)amino] ethanol (295 mg, 50% yield) as a colourless solid. ¹H NMR (500 MHz, DMSO-d₆) δ8.35 (s, 2H), 7.32 (t, J=5.8 Hz, 1H), 4.66 (t, J=5.6 Hz, 1H), 3.48 (q, J=6.1 Hz, 2H), 3.32-3.27 (m, 2H). m/z 219.9 [M+H, ⁸¹Br]⁺.

### Step 2

A solution of KF (76 mg, 1.3 mmol), 2-[(5-bromopyrimidin-2-yl)amino] ethanol (100 mg, 0.44 mmol) and Intermediate 7 (200 mg, 0.46 mmol) in 1,4-dioxane (4 mL) and water (1 mL) was degassed with nitrogen. Pd-118 (28 mg, 0.044 mmol) was added and the reaction mixture was degassed again and left to stir at rt for 16 h. The reaction mixture was partitioned between DCM (30 mL) and water (30 mL) and separated. The suspension in the aqueous layer was washed with DCM (20 mL) and the solids were collected and dried in a vacuum oven set at 40° C. for 1 h to give N-[6-[2-(2-hydroxyethylamino)pyrimidin-5-yl]-2-methoxy-3-pyridyl]-5-methyl-3-phenyl-isoxazole-4-carboxamide (89 mg, 43% yield) as a beige solid. ¹H NMR (500 MHZ, DMSO-d₆) δ9.44 (s, 1H), 8.93 (s, 2H), 8.26 (d, J=8.0 Hz, 1H), 7.77-7.66 (m, 2H), 7.59-7.50 (m, 3H), 7.48 (d, J=8.0 Hz, 1H), 7.38 (t, J=5.8 Hz, 1H), 4.70 (s, 1H), 3.88 (s, 3H), 3.54 (t, J=6.3 Hz, 2H), 3.44-3.37 (m, 2H), 2.66 (s, 3H). m/z 447.1 [M+H]⁺.

### Example 176 N-[2-(Dimethylamino)-6-imidazol-1-yl-3-pyridyl]-5-methyl-3-phenyl-isoxazole-4-carboxamide

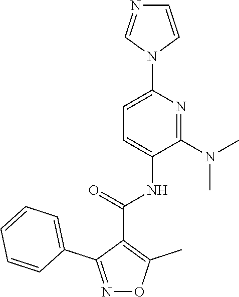 [SMILES: Cc1onc(-c2ccccc2)c1C(=O)Nc1ccc(-n2ccnc2)nc1N(C)C]

### Step 1

A stirring suspension of 2,6-dichloro-3-nitropyridine (2.00 g, 10.4 mmol) in THF (30 mL) at 0° C. was treated with K₂CO₃ (1.72 g, 12.4 mmol) followed by dropwise addition of a solution of dimethylamine (2 M in THF, 5.18 mL, 10.4 mmol) in THF (15 mL) over 40 min. The yellow suspension was left to stir at rt for 16 h. The reaction mixture was filtered and the filtrate concentrated under reduced pressure. The residue was adsorbed onto silica and purified by flash column chromatography (80 g silica, elution with 0-60% EtOAc/PE) to afford 6-chloro-N,N-dimethyl-3-nitropyridin-2-amine (816 mg, 37% yield) as a yellow solid. ¹H NMR (600 MHz, Chloroform-d) δ8.06 (d, J=8.4, 0.7 Hz, 1H), 6.62 (d, J=8.3 Hz, 1H), 3.05 (s, 6H). m/z 201.9 [M+H]⁺.

### Step 2

To a stirring solution of imidazole (276 mg, 4.05 mmol) in dry DMF (5 mL) at 0° C. was added sodium hydride (60% in mineral oil, 194 mg, 4.86 mmol) in portions over 10 min. The reaction mixture was stirred at 0° C. for 30 min before adding a solution of 6-chloro-N,N-dimethyl-3-nitro-pyridin-2-amine (816 mg, 4.05 mmol) in DMF (3 mL). The reaction mixture was left to stir at rt for 16 h. The reaction mixture was cooled in an ice-bath and treated with water (30 mL). The yellow precipitate formed was collected via vacuum filtration and the solid cake was washed with water and allowed to air dry to afford 6-imidazol-1-yl-N,N-dimethyl-3-nitro-pyridin-2-amine (809 mg, 81% yield) as a yellow solid, which was used in the next step without further purification. ¹H NMR (600 MHZ, DMSO-d₆) δ8.59 (d, J=1.1 Hz, 1H), 8.40 (d, J=8.6 Hz, 1H), 7.97 (t, J=1.4 Hz, 1H), 7.15-7.10 (m, 2H), 3.01 (s, 6H). m/z 233.7 [M+H]⁺.

### Step 3

To a stirring solution of 6-imidazol-1-yl-N,N-dimethyl-3-nitro-pyridin-2-amine (810 mg, 3.47 mmol) in EtOH (20 mL) was added 10% Pd on carbon (37 mg, 0.35 mmol). The flask was sealed with a rubber septum, purged with nitrogen followed by H₂ (balloon). The reaction mixture was left to stir at rt under hydrogen for 16 h. The reaction mixture was filtered through celite and washed with EtOH. The filtrate was concentrated under reduced pressure to afford 6-imidazol-1-yl-N²,N²-dimethyl-pyridine-2,3-diamine (626 mg, 84% yield) as a tan solid, which was used in the next step without further purification. ¹H NMR (600 MHZ, Chloroform-d) δ8.18 (s, 1H), 7.50 (s, 1H), 7.13 (s, 1H), 6.99 (d, J=8.1 Hz, 1H), 6.82 (d, J=8.0 Hz, 1H), 3.74 (s, 2H), 2.81 (s, 6H). m/z 204.0 [M+H]⁺.

### Step 4

To a stirring suspension of 6-imidazol-1-yl-N²,N²-dimethyl-pyridine-2,3-diamine (65 mg, 0.32 mmol) and (5-methyl-3-phenyl-isoxazol-4-yl)-(triazolo[4,5-b]pyridin-3-yl) methanone (98 mg, 0.32 mmol) in DMF (0.32 mL) was added DIPEA (0.17 mL, 0.96 mmol). The reaction mixture was heated at 60° C. for 16 h. The reaction mixture was partitioned between EtOAc (150 mL) and 1M NaOH (50 mL) and the phases were separated. The organic phase was washed with water (100 mL), brine (50 mL), dried over MgSO₄, filtered and concentrated under reduced pressure. The residue was adsorbed onto silica and purified on a Shimadzu MDAP (elution with a 30-95% MeCN/water+0.1% formic acid gradient) to afford an off-white solid. The material was adsorbed onto silica and purified again by flash column chromatography (4 g silica, elution with 0-80% EtOAc/PE) to afford N-[2-(dimethylamino)-6-imidazol-1-yl-3-pyridyl]-5-methyl-3-phenyl-isoxazole-4-carboxamide (45 mg, 34% yield) as a tan solid. ¹H NMR (600 MHZ, Chloroform-d) δ8.74 (d, J=8.5 Hz, 1H), 8.23 (s, 1H), 7.89 (s, 1H), 7.64-7.56 (m, 3H), 7.56-7.50 (m, 3H), 7.14 (s, 1H), 6.99 (d, J=8.5 Hz, 1H), 2.83 (s, 3H), 2.31 (s, 6H). m/z 389.0 [M+H]⁺.

### Example 177 N-(6-(1H-Imidazol-1-yl)-2-(methylamino)pyridin-3-yl)-5-methyl-3-phenylisoxazole-4-carboxamide

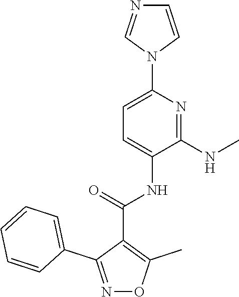 [SMILES: CNc1nc(-n2ccnc2)ccc1NC(=O)c1c(-c2ccccc2)noc1C]

To a stirring suspension of 6-imidazol-1-yl-N²-methyl-pyridine-2,3-diamine (synthesised in a similar manner to 6-imidazol-1-yl-N²,N²-dimethyl-pyridine-2,3-diamine described in Example 176) (61 mg, 0.32 mmol) and 1H-benzo[d] [1,2,3]triazol-1-yl 5-methyl-3-phenylisoxazole-4-carboxylate (98 mg, 0.31 mmol) in DMF (2 mL) was added DIPEA (0.17 mL, 0.96 mmol). The reaction mixture was heated at 60° C. for 16 h. The reaction mixture was allowed to cool to room temperature, then partitioned between EtOAc (150 mL) and 1M NaOH (50 mL) and separated. The organic phase was washed with water (100 mL), brine (50 mL), dried over MgSO₄, filtered and concentrated under reduced pressure. The residue was adsorbed onto silica and purified by ISCO Prep system in 3 batches (40 g silica, elution with 0-90% EtOAc/PE) to afford N-[6-imidazol-1-yl-2-(methylamino)-3-pyridyl]-5-methyl-3-phenyl-isoxazole-4-carboxamide (5.8 mg, 4% yield) as a tan solid. ¹H NMR (600 MHZ, Chloroform-d) δ8.25 (s, 1H), 7.69-7.59 (m, 5H), 7.57 (d, J=8.1 Hz, 1H), 7.52 (s, 1H), 7.12 (s, 1H), 6.57 (d, J=8.1 Hz, 1H), 6.50 (s, 1H), 3.99 (s, 1H), 2.85 (d, J=4.8 Hz, 3H), 2.81 (s, 3H). m/z 375.0 [M+H]⁺.

### Example 178 N-[2-Methoxy-6-(4,5,6,7-tetrahydroimidazo[4,5-c]pyridin-1-yl)-3-pyridyl]-5-methyl-3-phenyl-isoxazole-4-carboxamide hydrochloride

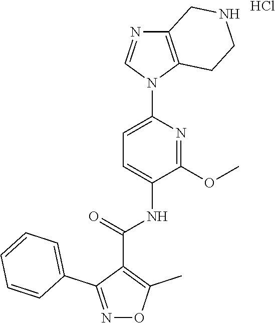 [SMILES: Cl ; COc1nc(-n2cnc3c2CCNC3)ccc1NC(=O)c1c(-c2ccccc2)noc1C]

### Step 1

To a solution of Intermediate 1 (750 mg, 1.93 mmol), tert-butyl 3,4,6,7-tetrahydroimidazo[4,5-c]pyridine-5-carboxylate (500 mg, 2.24 mmol) and L-proline (22 mg, 0.19 mmol) in DMSO (15 mL) was added K₂CO₃ (354 mg, 3.86 mmol) and the resultant suspension was sparged with nitrogen. Copper (I) iodide (37 mg, 0.19 mmol) was added and the mixture was sparged with nitrogen again before heating at 80° C. for 16 h. The reaction mixture was cooled to rt and additional copper(I) iodide (150 mg, 0.79 mmol) and L-proline (90 mg, 0.78 mmol) were added. The reaction mixture was sparged with nitrogen before reheating at 80° C. for an additional 48 h. The reaction mixture was poured onto ice-cold water (500 mL) and the brown solid was collected by vacuum filtration. The solid was dissolved in DCM, passed through a phase separator and concentrated to dryness.

Purification by flash silica column chromatography (40 g silica, elution with 50-100% EtOAc/PE) gave two regioisomers. The regiochemistry could not be assigned with confidence so the isomers were arbitrarily assigned as tert-butyl 1-[6-methoxy-5-[(5-methyl-3-phenyl-isoxazole-4-carbonyl)amino]-2-pyridyl]-6,7-dihydro-4H-imidazo[4,5-c]pyridine-5-carboxylate (140 mg, 13% yield) and tert-butyl 3-[6-methoxy-5-[(5-methyl-3-phenyl-isoxazole-4-carbonyl)amino]-2-pyridyl]-6,7-dihydro-4H-imidazo[4,5-c]pyridine-5-carboxylate (90 mg, 7% yield). The regioisomers could be clearly distinguished by NMR spectra and TLC.

tert-Butyl 1-[6-methoxy-5-[(5-methyl-3-phenyl-isoxazole-4-carbonyl)amino]-2-pyridyl]-6,7-dihydro-4H-imidazo[4,5-c]pyridine-5-carboxylate: R_{f} (EtOAc)=0.2. ¹H NMR (500 MHZ, Chloroform-d) δ8.79 (d, J=8.3 Hz, 1H), 8.04 (s, 1H), 7.69 (s, 1H), 7.68-7.62 (m, 3H), 7.62-7.55 (m, 2H), 6.86 (d, J=8.3 Hz, 1H), 4.54 (s, 2H), 3.74-3.68 (m, 2H), 3.63 (s, 3H), 3.00-2.86 (m, 2H), 2.83 (s, 3H), 1.48 (s, 9H). m/z 531.1 [M+H]⁺.

tert-Butyl 3-[6-methoxy-5-[(5-methyl-3-phenyl-isoxazole-4-carbonyl)amino]-2-pyridyl]-6,7-dihydro-4H-imidazo[4,5-c]pyridine-5-carboxylate: R/(EtOAc)=0.13. ¹H NMR (500 MHZ, Chloroform-d) δ8.80 (d, J=8.2 Hz, 1H), 7.90 (s, 1H), 7.70-7.58 (m, 6H), 6.92 (d, J=8.2 Hz, 1H), 4.79 (s, 2H), 3.82-3.72 (m, 2H), 3.68 (s, 3H), 2.86 (s, 3H), 2.77-2.66 (m, 2H), 1.49 (s, 9H). m/z 531.1 [M+H]⁺.

### Step 2

To a suspension of tert-butyl 1-[6-methoxy-5-[(5-methyl-3-phenyl-isoxazole-4-carbonyl)amino]-2-pyridyl]-6,7-dihydro-4H-imidazo[4,5-c]pyridine-5-carboxylate (140 mg, 0.26 mmol) in 1,4-dioxane (1 mL) was treated 4 M HCl (0.56 mL, 16 mmol) in 1,4-dioxane. An orange solution briefly resulted, before a precipitate formed. The mixture was stirred at rt for 1 h before diluted with TBME (30 mL) and the solid was collected by filtration and washed with TBME (2×30 mL). The solid was dried in a vacuum oven at 40° C. overnight to give N-[2-methoxy-6-(4,5,6,7-tetrahydroimidazo[4,5-c]pyridin-1-yl)-3-pyridyl]-5-methyl-3-phenyl-isoxazole-4-carboxamide hydrochloride (110 mg, 87% yield) as a light yellow solid. ¹H NMR (500 MHZ, DMSO-d₆) δ9.72 (s, 1H), 9.48 (s, 2H), 8.42 (s, 1H), 7.81-7.64 (m, 2H), 7.62-7.47 (m, 4H), 7.37 (d, J=7.3 Hz, 1H), 3.88 (s, 3H), 3.49-3.27 (m, 4H), 2.66 (s, 3H). One CH₂ under NMR solvent. m/z 431.1 [M+H for free base]⁺.

### Example 179 N-[2-Methoxy-6-(4,5,6,7-tetrahydroimidazo[4,5-c]pyridin-3-yl)-3-pyridyl]-5-methyl-3-phenyl-isoxazole-4-carboxamide dihydrochloride

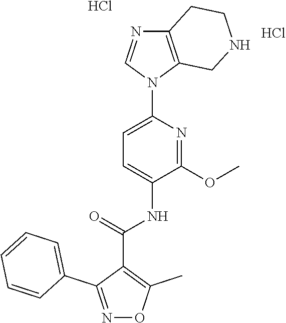 [SMILES: Cl ; COc1nc(-n2cnc3c2CNCC3)ccc1NC(=O)c1c(-c2ccccc2)noc1C ; Cl]

To a suspension of tert-butyl 3-[6-methoxy-5-[(5-methyl-3-phenyl-isoxazole-4-carbonyl)amino]-2-pyridyl]-6,7-dihydro-4H-imidazo[4,5-c]pyridine-5-carboxylate (90 mg, 0.17 mmol), synthesised as described in Example 178 (as noted, regiochemistry could not be assigned with confidence) in 1,4-dioxane (1.5 mL) was added 4 M HCl (0.83 mL, 24 mmol) in 1,4-dioxane. A light yellow solution resulted, which gradually formed a white suspension. After stirring for 1 h at rt, the reaction mixture was diluted with TBME (30 mL) and the solid was collected by filtration and washed with TBME (2×30 mL). The solid was dried in a vacuum oven at 40° C. overnight to give N-[2-methoxy-6-(4,5,6,7-tetrahydroimidazo[4,5-c]pyridin-3-yl)-3-pyridyl]-5-methyl-3-phenyl-isoxazole-4-carboxamide dihydrochloride (85 mg, 93% yield) as a colourless solid. ¹H NMR (500 MHZ, DMSO-d₆) δ9.74 (s, 1H), 9.46 (s, 2H), 8.80 (s, 1H), 8.42 (s, 1H), 7.84-7.62 (m, 2H), 7.62-7.49 (m, 4H), 7.47 (d, J=8.5 Hz, 1H), 4.66 (s, 2H), 3.90 (s, 3H), 3.47-3.42 (m, 2H), 2.97-2.83 (m, 2H), 2.65 (s, 3H). m/z 431.1 [M+H for free base]⁺.

### Example 180 N-(6-(2-(Aminomethyl)pyrimidin-5-yl)-2-methoxypyridin-3-yl)-1-methyl-4-phenyl-1H-1,2,3-triazole-5-carboxamide hydrochloride

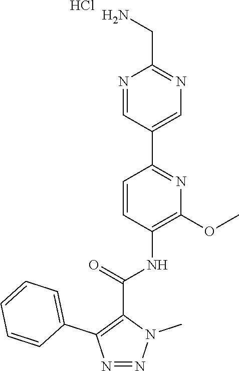 [SMILES: Cl ; COc1nc(-c2cnc(CN)nc2)ccc1NC(=O)c1c(-c2ccccc2)nnn1C]

### Step 1

N-(6-Bromo-2-methoxypyridin-3-yl)-1-methyl-4-phenyl-1H-1,2,3-triazole-5-carboxamide (0.16 g, 0.42 mmol), synthesised according to Example 154, B₂Pin₂ (0.14 g, 0.55 mmol), Pd(dppf)Cl₂ (35 mg, 0.040 mmol) and KOAc (124 mg, 1.26 mmol) were dissolved in 1,4-dioxane (5 mL). The reaction mixture was degassed with nitrogen for 5 mins before heating to 90° C. for 1 h. After cooling to rt, the reaction mixture was diluted with EtOAc (20 mL), washed with water (2×20 mL) and the organic layer dried over MgSO₄, filtered and concentrated under reduced pressure. The residue was purified by trituration with PE (20 mL) to afford N-(2-methoxy-6-(4,4,5,5-tetramethyl-1,3,2-dioxaborolan-2-yl)pyridin-3-yl)-1-methyl-4-phenyl-1H-1,2,3-triazole-5-carboxamide (98 mg, 51% yield) 1H NMR (500 MHZ, Chloroform-d) δ8.58 (d, J=7.8 Hz, 1H), 8.24 (br s, 1H), 7.62-7.65 (m, 2H), 7.49-7.57 (m, 3H), 7.47 (d, J=7.8 Hz, 1H), 4.42 (s, 3H), 3.68 (s, 3H), 1.35 (s, 12H). m/z 354.0 [M+H]⁺ for boronic acid.

### Step 2

N-(2-Methoxy-6-(4,4,5,5-tetramethyl-1,3,2-dioxaborolan-2-yl)pyridin-3-yl)-1-methyl-4-phenyl-1H-1,2,3-triazole-5-carboxamide (81 mg, 0.19 mmol) were added to a solution of Intermediate 11 (59 mg, 0.20 mmol) and KF (32 mg, 0.56 mmol)) in 1,4-dioxane (3 mL) and water (1 mL). The reaction mixture was degassed with nitrogen for 5 mins. Pd-118 (12 mg, 0.018 mmol) was quickly added and the mixture further degassed with nitrogen before stirring at 35° C. overnight. The reaction mixture was then concentrated under reduced pressure. The dark residue dissolved in DCM (10 mL) and washed with water (2×10 mL). The organic layer was dried over MgSO₄, filtered and concentrated under reduced pressure. The residue was purified by flash column chromatography (4 g, silica, 0-10% MeOH/DCM). CV's containing product were combined and evaporated under reduced pressure to afford tert-butyl ((5-(6-methoxy-5-(1-methyl-4-phenyl-1H-1,2,3-triazole-5-carboxamido)pyridin-2-yl)pyrimidin-2-yl)methyl) carbamate (30.2 mg, 30% yield) as a brown solid. ¹H NMR (500 MHz, Chloroform-d) δ9.22 (s, 2H), 8.76 (d, J=8.2 Hz, 1H), 8.21 (br s, 1H), 7.63-7.69 (m, 2H), 7.54-7.61 (m, 3H), 7.38 (d, J=8.1 Hz, 1H), 5.69 (br s, 1H), 4.64 (d, J=5.1 Hz, 2H), 4.44 (s, 3H), 3.71 (s, 3H), 1.49 (s, 9H). m/z 517.1 [M+H]⁺.

### Step 3

tert-Butyl ((5-(6-methoxy-5-(1-methyl-4-phenyl-1H-1,2,3-triazole-5-carboxamido)pyridin-2-yl)pyrimidin-2-yl)methyl) carbamate (30 mg, 0.06 mmol) was dissolved in DCM (5 mL), followed by addition of HCl (2 M in ether, 0.29 mL, 0.58 mmol). The reaction mixture was stirred at rt, monitoring the reaction progression by LCMS. Upon reaction completion, the reaction mixture was concentrated under reduced pressure to give N-(6-(2-(aminomethyl)pyrimidin-5-yl)-2-methoxypyridin-3-yl)-1-methyl-4-phenyl-1H-1,2,3-triazole-5-carboxamide hydrochloride (23.7 mg, 84% yield) as a bronze solid. ¹H NMR (500 MHZ, DMSO-d₆) δ10.45 (s, 1H), 9.52 (s, 2H), 8.38-8.49 (m, 4H), 7.89 (d, J=8.1 Hz, 1H), 7.80 (d, J=7.6 Hz, 2H), 7.46-7.53 (m, 2H), 7.38-7.46 (m, 1H), 4.39 (s, 2H), 4.18 (s, 3H), 3.99 (s, 3H). m/z 417.0 [M+H]⁺.

### Example 181 N-[2-Methoxy-6-[2-(morpholinomethyl)pyrimidin-5-yl]-3-pyridyl]-5-methyl-3-phenyl-isoxazole-4-carboxamide

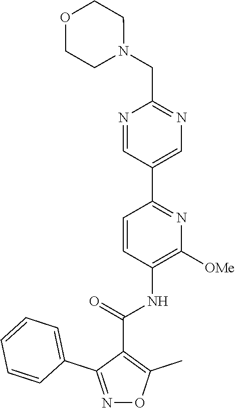 [SMILES: COc1nc(-c2cnc(CN3CCOCC3)nc2)ccc1NC(=O)c1c(-c2ccccc2)noc1C]

### Step 1

To a solution of Hünig's base (0.21 mL, 1.2 mmol) and Intermediate 8 (0.15 g, 0.60 mmol) THF (6 mL) was added drop-wise morpholine (60 μL, 0.65 mmol). The solution gradually turned into a suspension, which was stirred at room temperature overnight. The reaction mixture was diluted with additional THF (5 mL) and filtered by gravity through filter paper. The filtrate was concentrated under reduced pressure and the solid further dried in vacuum oven (40° C.) for 2 h to give 4-[(5-bromopyrimidin-2-yl)methyl] morpholine (0.17 g, 100% yield) as a beige solid, which was used without further purification. ¹H NMR (500 MHZ, Chloroform-d) δ8.76 (s, 2H), 3.77-3.73 (m, 6H), 2.60-2.52 (m, 4H).

### Step 2

A solution of KF (103 mg, 1.78 mmol), 4-[(5-bromopyrimidin-2-yl)methyl] morpholine (170 mg, 0.590 mmol) and Intermediate 7 (271 mg, 0.590 mmol) in 1,4-dioxane (5.7 mL) and water (1.4 mL) was degassed with nitrogen for 15 min. Pd-118 (38 mg, 0.060 mmol) was quickly added, the solution degassed again for 10 min and the mixture left to stir at room temperature overnight. The mixture was heated at 40° C. for 4 h. The reaction mixture was diluted with DCM (20 mL) and washed with water (20 mL). The organic phase was dried over MgSO₄, filtered and concentrated under reduced pressure to give the crude product, which was purified by automated column chromatography on silica (12 g, gradient from 0-10% MeOH/DCM over 30 CV) to give N-[2-methoxy-6-[2-(morpholinomethyl)pyrimidin-5-yl]-3-pyridyl]-5-methyl-3-phenyl-isoxazole-4-carboxamide (145 mg, 48% yield) as a beige solid. ¹H NMR (500 MHZ, Chloroform-d) δ9.21 (s, 2H), 8.75 (d, J=8.1 Hz, 1H), 7.78 (s, 1H), 7.67-7.60 (m, 3H), 7.60-7.54 (m, 2H), 7.34 (d, J=8.1 Hz, 1H), 3.84 (s, 2H), 3.77 (t, J=4.6 Hz, 4H), 3.68 (s, 3H), 2.82 (s, 3H), 2.60 (t, J=4.6 Hz, 4H). m/z 487.1 [M+H]⁺.

### Example 182 N-[6-[4-(Aminomethyl)phenyl]-2-methoxy-3-pyridyl]-5-methyl-3-phenyl-isoxazole-4-carboxamide hydrochloride

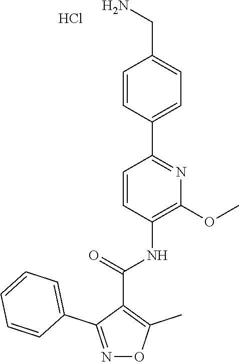 [SMILES: COc1nc(-c2ccc(CN)cc2)ccc1NC(=O)c1c(-c2ccccc2)noc1C ; Cl]

A suspension of KF (95 mg, 1.6 mmol), 4-bromobenzylamine (70 μL, 0.55 mmol) and Intermediate 7 (0.25 g, 0.55 mmol) in 1,4-dioxane (5 mL) and water (1.25 mL) was degassed with nitrogen for 15 min. Pd-118 (35 mg, 0.054 mmol) was quickly added, the solution degassed again for 10 min and the mixture left to stir at room temperature overnight. Then, water (10 mL) was added and the reaction mixture extracted with DCM (3×10 mL). The combined organics were washed with brine (10 mL), dried over MgSO₄ and filtered before concentration to dryness. Purification by flash silica column chromatography on an ISCO system (elution with 10% MeOH/DCM gradient) yielded the free base that was subsequently treated with 1.2 eq. of HCl (4 N in 1,4-dioxane) in DCM to give N-[6-[4-(aminomethyl)phenyl]-2-methoxy-3-pyridyl]-5-methyl-3-phenyl-isoxazole-4-carboxamide hydrochloride (74 mg, 29% yield) as brownish solid, after evaporation of volatile residues. ¹H NMR (400 MHZ, DMSO-d₆) δ9.53 (s, 1H), 8.51 (s, 3H), 8.11 (d, J=7.9 Hz, 2H), 7.72 (d, J=6.7 Hz, 2H), 7.66-7.53 (m, 6H), 7.47 (d, J=8.1 Hz, 1H), 4.07 (q, J=5.6 Hz, 2H), 3.92 (s, 3H), 2.67 (s, 3H). m/z 398.0 [M-NH₃]⁺, 416.0 [M-NH₂+OH+H]⁺.

### Example 183 N-[2-Methoxy-6-[4-(methylaminomethyl)phenyl]-3-pyridyl]-5-methyl-3-phenyl-isoxazole-4-carboxamide hydrochloride

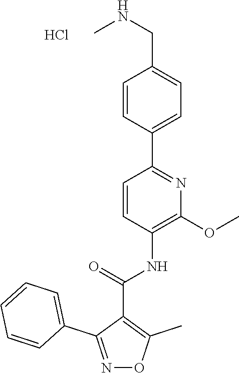 [SMILES: CNCc1ccc(-c2ccc(NC(=O)c3c(-c4ccccc4)noc3C)c(OC)n2)cc1 ; Cl]

As described for the synthesis of Example 182, substituting 1-(4-bromophenyl)-N-methyl-methanamine for 4-bromobenzylamine to give N-[2-methoxy-6-[4-(methylaminomethyl)phenyl]-3-pyridyl]-5-methyl-3-phenyl-isoxazole-4-carboxamide hydrochloride (100 mg, 35% yield) as brown solid ¹H NMR (400 MHz, DMSO-d₆) δ9.53 (s, 1H), 8.96 (s, 2H), 8.38 (s, 1H), 8.14 (d, J=8.0 Hz, 2H), 7.72 (d, J=6.8 Hz, 2H), 7.69-7.46 (m, 6H), 4.17 (s, 2H), 3.93 (s, 3H), 3.57 (s, 3H), 2.67 (s, 3H). m/z 429.2 [M+H for free base]⁺.

### Example 184 N-(6-(2-(Aminomethyl)pyrimidin-5-yl)-2-methoxypyridin-3-yl)-4-(4-fluorophenyl)-1-methyl-1H-1,2,3-triazole-5-carboxamide hydrochloride

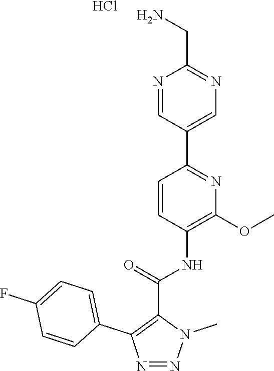 [SMILES: Cl ; COc1nc(-c2cnc(CN)nc2)ccc1NC(=O)c1c(-c2ccc(F)cc2)nnn1C]

As described for the synthesis of Example 180, substituting N-(6-bromo-2-methoxypyridin-3-yl)-4-(4-fluorophenyl)-1-methyl-1H-1,2,3-triazole-5-carboxamide (synthesised using appropriate starting materials by the method described in Step 1 of Example 154) for N-(6-bromo-2-methoxypyridin-3-yl)-1-methyl-4-phenyl-1H-1,2,3-triazole-5-carboxamide to give N-(6-(2-(aminomethyl)pyrimidin-5-yl)-2-methoxypyridin-3-yl)-4-(4-fluorophenyl)-1-methyl-1H-1,2,3-triazole-5-carboxamide hydrochloride (35 mg, 47% yield) as a yellow solid. ¹H NMR (500 MHZ, DMSO-d₆) δ10.46 (s, 1H), 9.52 (s, 2H), 8.35-8.54 (m, 4H), 7.89 (d, J=8.0 Hz, 1H), 7.80-7.87 (m, 2H), 7.35 (t, J=8.6 Hz, 2H), 4.40 (s, 2H), 4.19 (s, 3H), 4.01 (s, 3H). m/z 435.1 [M+H]⁺.

### Example 185 N-[6-[2-(Aminomethyl)pyrimidin-5-yl]-2-methoxy-3-pyridyl]-3-(4-fluorophenyl)-5-methyl-isoxazole-4-carboxamide hydrochloride

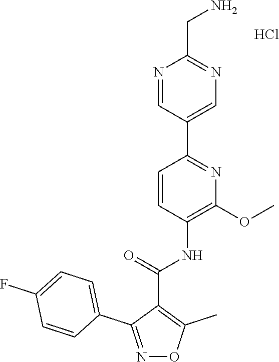 [SMILES: COc1nc(-c2cnc(CN)nc2)ccc1NC(=O)c1c(-c2ccc(F)cc2)noc1C ; Cl]

### Step 1

To a microwave vial under an inert atmosphere was added Intermediate 12 (0.49 g, 1.2 mmol), B₂Pin₂ (0.46 g, 1.8 mmol) and 1,4-dioxane (9 mL). The solution was degassed with nitrogen for 15 min before Pd(dppf)Cl₂ (complex with DCM, 0.10 g, 0.12 mmol) and KOAc (355 mg, 3.62 mmol) were quickly added. The mixture was again degassed with nitrogen for 15 min before being heated to 80° C. for 1 h. The reaction mixture was allowed to cool and was diluted with EtOAc (20 mL). The solution was washed with water (20 mL), dried over MgSO₄, filtered and concentrated under reduced pressure to give the crude product which was purified by trituration with PE. The suspension was left to settle, the supernatant PE was removed and the trituration repeated with more PE. The resulting brown powder was dried under reduced pressure to give 3-(4-fluorophenyl)-N-[2-methoxy-6-(4,4,5,5-tetramethyl-1,3,2-dioxaborolan-2-yl)-3-pyridyl]-5-methyl-isoxazole-4-carboxamide (0.50 g, 76% yield) as a brown powder. ¹H NMR (500 MHZ, Chloroform-d) δ8.59 (d, J=7.8 Hz, 1H), 7.76 (s, 1H), 7.64-7.60 (m, 2H), 7.46 (d, J=7.8 Hz, 1H), 7.25-7.21 (m, 2H), 3.74 (s, 3H), 2.81 (s, 3H), 1.35 (s, 12H). m/z 372.1 [M+H]⁺ for boronic acid.

### Step 2

A solution of KF (77 mg, 1.3 mmol), Intermediate 11 (0.13 mL, 0.44 mmol) and 3-(4-fluorophenyl)-N-[2-methoxy-6-(4,4,5,5-tetramethyl-1,3,2-dioxaborolan-2-yl)-3-pyridyl]-5-methyl-isoxazole-4-carboxamide (0.20 g, 0.44 mmol) in 1,4-dioxane (3.8 mL) and water (0.9 mL) was degassed with nitrogen for 15 min. Pd-118 (29 mg, 0.044 mmol) was quickly added, the solution degassed again for 10 min and the mixture left to stir at room temperature overnight. Then, water (10 mL) was added and the reaction mixture extracted with DCM (3×10 mL). The combined organics were washed with brine (10 mL), dried over MgSO₄ and filtered before concentration to dryness. The crude was then treated with HCl (4N in 1,4-dioxane, 0.22 mL, 0.88 mmol) and, once the starting material was completely deprotected, the reaction mixture was basified and extracted with DCM. Purification by flash silica column chromatography on an ISCO system (elution with 8% MeOH/DCM gradient) yielded the free base that was subsequently treated with 1.2 eq. of HCl (4N in 1,4-dioxane) in DCM to give N-[6-[2-(aminomethyl)pyrimidin-5-yl]-2-methoxy-3-pyridyl]-3-(4-fluorophenyl)-5-methyl-isoxazole-4-carboxamide hydrochloride (80 mg, 37% yield) as brown solid after evaporation of solvents. ¹H NMR (400 MHZ, DMSO-d₆) δ9.65 (s, 1H), 9.50 (s, 2H), 8.57 (s, 3H), 8.44 (s, 1H), 7.85 (d, J=8.1 Hz, 1H), 7.78 (dd, J=8.5, 5.4 Hz, 2H), 7.41 (t, J=8.7 Hz, 2H), 4.38 (q, J=5.9 Hz, 2H), 3.97 (s, 3H), 2.67 (s, 3H). m/z 435.1 [M+H]⁺.

### Example 186 N-(2-Methoxy-6-(7-methyl-5,6,7,8-tetrahydroimidazol[1,5-a]pyrazin-1-yl)pyridin-3-yl)-5-methyl-3-phenylisoxazole-4-carboxamide

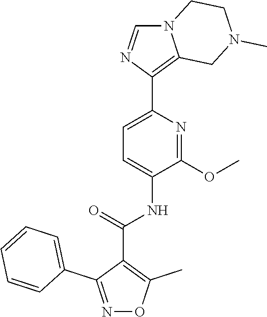 [SMILES: COc1nc(-c2ncn3c2CN(C)CC3)ccc1NC(=O)c1c(-c2ccccc2)noc1C]

### Step 1

A 20 mL microwave vial was charged with Intermediate 7 (0.40 g, 0.92 mmol), tert-butyl 1-bromo-6,8-dihydro-5H-imidazo[1,5-a]pyrazine-7-carboxylate (417 mg, 1.38 mmol), KF (160 mg, 2.76 mmol), 1,4-dioxane (9 mL) and water (3 mL). The reaction mixture was degassed with nitrogen for 5 mins. Pd-118 (60 mg, 0.091 mmol) was quickly added and the mixture further degassed with nitrogen before stirring at 35° C. overnight. The reaction mixture was then concentrated under reduced pressure. The dark residue dissolved in DCM (20 mL) and washed with water (2×10 mL). The organic layer was dried over MgSO₄, filtered and concentrated under reduced pressure. The residue was purified by flash column chromatography (12 g, silica, 0-100% EtOAc/PE+1% Et₃N over 40 CV's). CV's containing product were combined and evaporated under reduced pressure. The residue was then triturated with hot MeOH (3 mL) and the precipitate dried under vacuum at 40° C. to afford tert-butyl 1-(6-methoxy-5-(5-methyl-3-phenylisoxazole-4-carboxamido)pyridin-2-yl)-5,6-dihydroimidazo[1,5-a]pyrazine-7 (8H)-carboxylate (0.19 g, 38% yield) as a cream solid. ¹H NMR (500 MHZ, Chloroform-d) δ8.68 (d, J=8.2 Hz, 1H), 7.61-7.70 (m, 3H), 7.53-7.61 (m, 4H), 7.46 (s, 1H), 5.06 (s, 2H), 4.06 (t, J=5.4 Hz, 2H), 3.83 (m, 2H), 3.68 (s, 3H), 2.82 (s, 3H), 1.48 (s, 9H). m/z 531.2 [M+H]⁺.

### Step 2

A 10 mL microwave vial was charged with tert-butyl 1-(6-methoxy-5-(5-methyl-3-phenylisoxazole-4-carboxamido)pyridin-2-yl)-5,6-dihydroimidazo[1,5-a]pyrazine-7 (8H)-carboxylate (95 mg, 0.18 mmol), paraformaldehyde (22 mg, 0.72 mmol) and formic acid (3 mL). The reaction mixture was heated to 95° C. and stirred for 2 h. The reaction mixture was cooled, diluted with DCM (15 mL) and washed sequentially with 2 M NaOH (2×15 mL), water (2×15 mL) and brine (1×15 mL). The organic layer was dried over MgSO₄, filtered and concentrated under reduced pressure. The residue was purified by flash column chromatography (4 g, silica, 0-10% MeOH/DCM over 25 CV's). CV's containing product were combined and evaporated under reduced pressure. The precipitate was then dried under vacuum at 40° C. to afford N-(2-methoxy-6-(7-methyl-5,6,7,8-tetrahydroimidazol[1,5-a]pyrazin-1-yl)pyridin-3-yl)-5-methyl-3-phenylisoxazole-4-carboxamide (49 mg, 58% yield) as an orange solid. ¹H NMR (500 MHZ, Chloroform-d) δ8.66 (d, J=8.2 Hz, 1H), 7.62-7.68 (m, 3H), 7.54-7.62 (m, 4H), 7.43 (s, 1H), 4.10 (t, J=5.4 Hz, 2H), 4.07 (s, 2H), 3.64 (s, 3H), 2.79-2.86 (m, 5H) 2.50 (s, 3H). m/z 445.2 [M+H]⁺.

### Example 187 N-(2-Methoxy-6-(5,6,7,8-tetrahydroimidazo[1,5-a]pyrazin-1-yl)pyridin-3-yl)-5-methyl-3-phenylisoxazole-4-carboxamide dihydrochloride

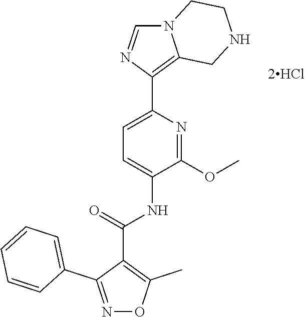 [SMILES: COc1nc(-c2ncn3c2CNCC3)ccc1NC(=O)c1c(-c2ccccc2)noc1C]

A 10 mL microwave vial was charged with tert-butyl 1-(6-methoxy-5-(5-methyl-3-phenylisoxazole-4-carboxamido)pyridin-2-yl)-5,6-dihydroimidazo[1,5-a]pyrazine-7 (8H)-carboxylate (synthesised according to Example 186) (81 mg, 0.15 mmol), HCl (2 M in ether, 0.76 mL, 1.5 mmol) and DCM (5 mL). The reaction mixture was stirred at rt overnight. The reaction mixture was concentrated under reduced pressure and the precipitate dried under vacuum at 40° C. to afford N-(2-methoxy-6-(5,6,7,8-tetrahydroimidazo[1,5-a]pyrazin-1-yl)pyridin-3-yl)-5-methyl-3-phenylisoxazole-4-carboxamide dihydrochloride (77 mg, 95%) as a light yellow solid. ¹H NMR (500 MHZ, DMSO-d₆) δ9.87 (br s, 2H), 9.54 (s, 1H), 8.41 (br s, 1H), 8.32 (br s, 1H), 7.72 (d, J=6.8 Hz, 2H), 7.50-7.63 (m, 4H), 4.81 (s, 2H), 4.43 (t, J=5.7 Hz, 2H), 3.90 (s, 3H), 2.66 (s, 3H). One CH₂ signal overlapping with NMR solvent. m/z 431.1 [M+H for free base]⁺.

### Example 188 N-[6-[2-(Azetidin-3-ylamino)pyrimidin-5-yl]-2-methoxy-3-pyridyl]-5-methyl-3-phenyl-isoxazole-4-carboxamide

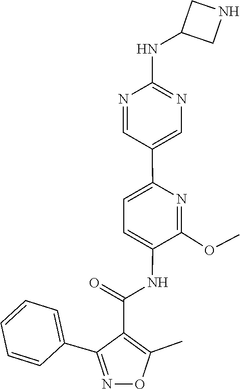 [SMILES: COc1nc(-c2cnc(NC3CNC3)nc2)ccc1NC(=O)c1c(-c2ccccc2)noc1C]

### Step 1

To a 25 mL microwave vial was added 5-bromo-2-chloropyrimidine (0.50 g, 2.6 mmol), DIPEA (0.54 mL, 3.1 mmol), tert-butyl 3-aminoazetidine-1-carboxylate (0.17 mL, 2.9 mmol) and MeCN (2.5 mL). The vial was sealed and heated at 80° C. for 16 h. The volatiles were removed under reduced pressure and the residue was re-dissolved in DCM (20 mL) and partitioned with 1M NaOH (20 mL). The aqueous layer was further extracted with DCM (2×20 mL). The combined organic extracts were dried over MgSO₄, filtered and concentrated under reduced pressure to give tert-butyl 3-[(5-bromopyrimidin-2-yl)amino]azetidine-1-carboxylate (1.00 g, 100% yield) as an orange solid, which was used in the next step without further purification. ¹H NMR (500 MHZ, Chloroform-d) δ8.29 (s, 2H), 5.51 (d, J=6.8 Hz, 1H), 4.60 (dtd, J=12.4, 7.3, 5.2 Hz, 1H), 4.29 (dd, J=9.2, 7.5 Hz, 2H), 3.78 (dd, J=9.2, 5.1 Hz, 2H), 1.44 (s, 9H). m/z 272.9/274.9 [M-C(CH₃)₃+H]⁺.

### Step 2

A 25 mL microwave vial was charged with Intermediate 7 (0.40 g, 0.92 mmol), tert-butyl 3-[(5-bromopyrimidin-2-yl)amino] azetidine-1-carboxylate (0.36 g, 1.1 mmol), KF (0.16 g, 2.8 mmol), 1,4-dioxane (8 mL) and water (2 mL). The mixture was degassed for 10 min before adding Pd-118 (0.06 g, 0.09 mmol) and the vial was sealed and stirred at room temperature for 14 h. Water (20 mL) was added and the mixture was extracted with DCM (3×20 mL). The combined organic extracts were dried over MgSO₄, filtered and concentrated under reduced pressure. The solids were triturated in EtOAc and the solids were collected via vacuum filtration to afford tert-butyl 3-((5-(6-methoxy-5-(5-methyl-3-phenylisoxazole-4-carboxamido)pyridin-2-yl)pyrimidin-2-yl)amino) azetidine-1-carboxylate (0.22 g, 40% yield) as an off-white solid, which was used in the next step without further purification. ¹H NMR (500 MHZ, Chloroform-d) δ8.86 (s, 2H), 8.69 (d, J=8.1 Hz, 1H), 7.73 (s, 1H), 7.63 (t, J=7.2 Hz, 3H), 7.60-7.52 (m, 2H), 7.17 (d, J=8.1 Hz, 1H), 5.53 (d, J=6.7 Hz, 1H), 4.76-4.64 (m, 1H), 4.33 (t, J=8.3 Hz, 2H), 3.82 (dd, J=9.1, 5.2 Hz, 2H), 3.67 (s, 3H), 2.83 (s, 3H), 1.45 (s, 9H). m/z 558.2 [M+H]⁺.

### Step 3

To a suspension of tert-butyl 3-((5-(6-methoxy-5-(5-methyl-3-phenylisoxazole-4-carboxamido)pyridin-2-yl)pyrimidin-2-yl)amino) azetidine-1-carboxylate (0.15 g, 0.26 mmol) in DCM (4 mL) was added trifluoroacetic acid (1.0 mL, 13 mmol) and the reaction mixture was stirred at room temperature for 16 h. The reaction mixture was cooled in an ice bath and treated with 2 M NaOH (10 mL) and the mixture was left to stir in the ice bath for 30 min. The mixture was diluted further with DCM (20 mL) and water (20 mL). The layers were separated and the aqueous phase was further extracted with DCM (2×20 mL). The combined organic extracts were washed with brine (20 mL), dried over MgSO₄, filtered and concentrated under reduced pressure to afford a solid, which was triturated in MeOH to afford N-[6-[2-(azetidin-3-ylamino)pyrimidin-5-yl]-2-methoxy-3-pyridyl]-5-methyl-3-phenyl-isoxazole-4-carboxamide (0.09 g, 71% yield) as a yellow solid. ¹H NMR (500 MHZ, DMSO-d₆) δ 9.45 (s, 1H), 8.95 (s, 2H), 8.26 (d, J=8.1 Hz, 1H), 8.00 (d, J=6.9 Hz, 1H), 7.71 (d, J=6.9 Hz, 2H), 7.55 (q, J=6.5 Hz, 3H), 7.49 (d, J=8.1 Hz, 1H), 4.75-4.62 (m, 1H), 3.89 (s, 3H), 3.59 (t, J=7.4 Hz, 2H), 3.50 (t, J=7.3 Hz, 2H), 2.66 (s, 3H). m/z 458.1 [M+H]⁺.

### Example 189 N-[6-[2-(Azetidin-3-yloxy)pyrimidin-5-yl]-2-methoxy-3-pyridyl]-5-methyl-3-phenyl-isoxazole-4-carboxamide

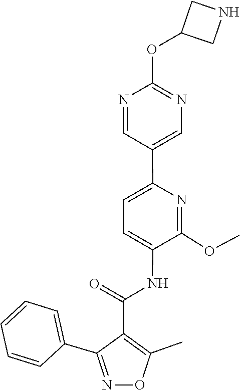 [SMILES: COc1nc(-c2cnc(OC3CNC3)nc2)ccc1NC(=O)c1c(-c2ccccc2)noc1C]

### Step 1

A solution of tert-butyl 3-hydroxyazetidine-1-carboxylate (0.68 g, 3.9 mmol) in THF (15 mL) was cooled in an ice-bath. Sodium hydride (60% in mineral oil, 0.17 g, 4.1 mmol) was added and the suspension was stirred for 30 min at 0° C. 5-Bromo-2-chloropyrimidine (0.76 g, 3.9 mmol) was added and the reaction mixture was left to stir at room temperature for 30 min. The reaction was quenched with water (1 mL), and the mixture was diluted with 1M NaOH (20 mL), brine (20 mL) and DCM (50 mL). The aqueous layer was further extracted with DCM (2×50 mL). Combined organic extracts were dried over MgSO₄, filtered and concentrated to give tert-butyl 3-(5-bromopyrimidin-2-yl)oxyazetidine-1-carboxylate (1.3 g, 90% yield) as an off-white solid, which was used in the next step without further purification. ¹H NMR (500 MHZ, Chloroform-d) δ8.53 (s, 2H), 5.27 (td, J=6.7, 3.3 Hz, 1H), 4.31 (dd, J=10.1, 6.6 Hz, 2H), 4.03 (dd, J=10.1, 4.3 Hz, 2H), 1.44 (s, 9H). m/z 273.9/275.9 [M-C(CH₃)₃+H]⁺.

### Step 2

A 25 mL microwave vial was charged with Intermediate 7 (0.40 g, 0.92 mmol), tert-butyl 3-(5-bromopyrimidin-2-yl)oxyazetidine-1-carboxylate (0.36 g, 1.1 mmol), KF (0.16 g, 2.8 mmol), 1,4-dioxane (8 mL) and water (2 mL). The mixture was degassed for 10 min before adding Pd-118 (0.06 g, 0.09 mmol) and the vial was sealed and stirred at room temperature for 14 h. Water (20 mL) was added and the mixture was extracted with DCM (3× 20 mL). The combined organic extracts were dried over MgSO₄, filtered and concentrated under reduced pressure. The material was triturated in EtOAc and solids were collected via vacuum filtration to afford tert-butyl 3-[5-[6-methoxy-5-[(5-methyl-3-phenyl-isoxazole-4-carbonyl)amino]-2-pyridyl]pyrimidin-2-yl] oxyazetidine-1-carboxylate (0.22 g, 40% yield) as an off-white solid, which was used in the next step without further purification. ¹H NMR (500 MHz, Chloroform-d) δ9.03 (s, 2H), 8.74 (d, J=8.1 Hz, 1H), 7.77 (s, 1H), 7.63 (d, J=6.9 Hz, 3H), 7.61-7.53 (m, 2H), 7.29-7.26 (m, 1H), 5.35 (ddt, J=10.1, 5.4, 2.7 Hz, 1H), 4.35 (dd, J=9.8, 6.8 Hz, 2H), 4.07 (dd, J=10.0, 4.2 Hz, 2H), 3.69 (s, 3H), 2.84 (s, 3H), 1.45 (s, 9H). m/z 559.2 [M+H]⁺

### Step 3

To a suspension of tert-butyl 3-[5-[6-methoxy-5-[(5-methyl-3-phenyl-isoxazole-4-carbonyl)amino]-2-pyridyl]pyrimidin-2-yl] oxyazetidine-1-carboxylate (0.22 g, 0.39 mmol) in DCM (4 mL) was added trifluoroacetic acid (1.0 mL, 13 mmol) and the reaction mixture was stirred at room temperature for 16 h. The reaction mixture was cooled in an ice bath and treated with 2M NaOH (10 mL) and the mixture was left to stir in the ice bath for 30 min. The mixture was diluted further with DCM (20 mL) and water (20 mL). The layers were separated and the aqueous phase was further extracted with DCM (2×20 mL). The combined organic extracts were washed with brine (20 mL), dried over MgSO₄, filtered and concentrated under reduced pressure. The residue was adsorbed onto silica and purified by flash column chromatography (12 g silica, elution with a 5-10% MeOH/DCM+1% Et₃N gradient) to afford N-[6-[2-(azetidin-3-yloxy)pyrimidin-5-yl]-2-methoxy-3-pyridyl]-5-methyl-3-phenyl-isoxazole-4-carboxamide (0.11 g, 58% yield) as a yellow solid. ¹H NMR (500 MHZ, DMSO-d₆) δ9.52 (s, 1H), 9.25 (s, 2H), 8.37 (d, J=8.1 Hz, 1H), 7.68-7.61 (m, 3H), 7.61-7.55 (m, 3H), 5.55-5.46 (m, 1H), 4.21 (dd, J=10.9, 6.8 Hz, 2H), 3.95 (dd, J=11.1, 5.4 Hz, 2H), 3.91 (s, 3H), 2.66 (s, 3H). m/z 459.1 [M+H]⁺.

### Example 190 N-[2-Methoxy-6-[6-(methylaminomethyl)-3-pyridyl]-3-pyridyl]-5-methyl-3-phenyl-isoxazole-4-carboxamide

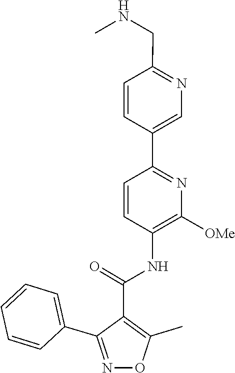 [SMILES: CNCc1ccc(-c2ccc(NC(=O)c3c(-c4ccccc4)noc3C)c(OC)n2)cn1]

### Step 1

To a solution of 5-bromopyridine-2-carbaldehyde (465 mg, 2.45 mmol) in MeOH (10 mL) was added 2M methylamine in DCM (1.38 mL, 2.75 mmol). The reaction mixture was stirred at RT for 2 hours. Additional 2 M methylamine in DCM (0.14 mL, 0.28 mmol) was added and the mixture left to stir overnight at room temperature. Sodium borohydride (151 mg, 3.99 mmol) was added portion-wise and the mixture left to stir at room temperature for 30 min. 2 M NaOH (aq, 10 mL) was added and the crude mixture concentrated (removal of MeOH) under reduced pressure. The resulting aqueous phase was extracted with DCM (3×20 mL) and EtOAc (2×40 mL), the combined organic extracts dried over MgSO₄ and concentrated under reduced pressure to give 1-(5-bromo-2-pyridyl)-N-methyl-methanamine (487 mg, 91% yield) as a light yellow oil, which was used without further purification. ¹H NMR (500 MHZ, Chloroform-d) δ8.59 (s, 1H), 7.75 (d, J=8.3 Hz, 1H), 7.21 (d, J=8.3 Hz, 1H), 3.81 (s, 2H), 2.45 (s, 3H). m/z 203.0 [M+H, ⁸¹Br]⁺.

### Step 2

A solution of KF (95 mg, 1.6 mmol), 1-(5-bromo-2-pyridyl)-N-methyl-methanamine (115 mg, 0.550 mmol) and Intermediate 7 (250 mg, 0.550 mmol) in 1,4-dioxane (5 mL) and water (1.25 mL) was degassed with nitrogen for 15 min. Pd-118 (35 mg, 0.050 mmol) was quickly added, the solution degassed again for 10 min and the mixture left to stir overnight at room temperature. The mixture was heated to 40° C. for 3 h. Additional Pd-118 (35 mg, 0.050 mmol) and KF (95 mg, 1.6 mmol) were added and the mixture left to stir for 4 h. Finally, the mixture was heated to 70° C. and left to stir overnight. The reaction mixture was diluted with DCM (20 mL), washed with water (20 mL), dried over MgSO₄, filtered and concentrated under reduced pressure to give the crude product, which was purified by automated column chromatography on silica (12 g, gradient from 0-10% MeOH/DCM). The resulting crude product was further purified by trituration in EtOAc (2×4 mL) to give N-[2-methoxy-6-[6-(methylaminomethyl)-3-pyridyl]-3-pyridyl]-5-methyl-3-phenyl-isoxazole-4-carboxamide (71 mg, 29% yield) as a beige solid. ¹H NMR (500 MHZ, Chloroform-d) δ9.13 (s, 1H), 8.73 (d, J=8.2 Hz, 1H), 8.21 (d, J=8.2 Hz, 1H), 7.77 (s, 1H), 7.68-7.61 (m, 3H), 7.61-7.54 (m, 2H), 7.42 (d, J=8.1 Hz, 1H), 7.34 (d, J=8.1 Hz, 1H), 4.08 (s, 2H), 3.69 (s, 3H), 2.84 (s, 3H), 2.61 (s, 3H). m/z 430.1 [M+H]⁺.

### Example 191 N-[2-Methoxy-6-(2-methoxy-4-methyl-pyrimidin-5-yl)-3-pyridyl]-5-methyl-3-phenyl-isoxazole-4-carboxamide

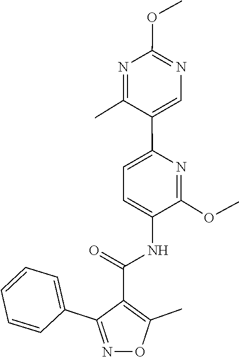 [SMILES: COc1ncc(-c2ccc(NC(=O)c3c(-c4ccccc4)noc3C)c(OC)n2)c(C)n1]

### Step 1

To an ice-cooled solution of MeOH (10 mL) was added in portions sodium (92 mg, 4.0 mmol). The mixture was stirred until all the sodium metal has fully dissolved. 5-Bromo-2-chloro-4-methyl-pyrimidine (0.42 g, 2.0 mmol) was added to the solution and the reaction mixture was stirred for 16 h. The solvent was removed under reduced pressure and water (20 mL) was added to the residue. The solution was extracted with EtOAc (3×20 mL) and the organic extracts were dried over MgSO₄, filtered and concentrated under reduced pressure to afford 5-bromo-2-methoxy-4-methyl-pyrimidine (0.34 g, 79% yield) as a yellow oil, which was used in the next step without further purification. ¹H NMR (500 MHZ, Chloroform-d) δ8.43 (s, 1H), 3.98 (s, 3H), 2.55 (s, 3H). m/z 203.0/204.8 [M+H]⁺.

### Step 2

A 10 mL microwave vial was charged with Intermediate 7 (0.20 g, 0.46 mmol), 5-bromo-2-methoxy-4-methyl-pyrimidine (0.11 g, 0.55 mmol), KF (80 mg, 1.4 mmol), 1,4-dioxane (4 mL) and water (1 mL). The mixture was degassed for 10 min before adding Pd-118 (0.03 g, 0.05 mmol) and the vial was sealed and stirred at room temperature for 2 h. Water (10 mL) was added and the mixture was extracted with DCM (3×10 mL). The combined organic extracts were dried over MgSO₄, filtered and concentrated under reduced pressure. The residue was adsorbed onto silica and purified by flash column chromatography (12 g silica, elution with a 0-50% EtOAc/PE gradient) to afford a solid, which was further triturated with EtOAc/PE to afford N-[2-methoxy-6-(2-methoxy-4-methyl-pyrimidin-5-yl)-3-pyridyl]-5-methyl-3-phenyl-isoxazole-4-carboxamide (0.11 g, 51% yield) as a yellow solid. ¹H NMR (500 MHz, Chloroform-d) δ8.73 (d, J=8.0 Hz, 1H), 8.51 (s, 1H), 7.76 (s, 1H), 7.69-7.54 (m, 5H), 7.00 (d, J=8.0 Hz, 1H), 4.03 (s, 3H), 3.63 (s, 3H), 2.84 (s, 3H), 2.58 (s, 3H). m/z 432.1 [M+H]⁺.

### Example 192 N-[2-Methoxy-6-[4-methyl-2-(methylamino)pyrimidin-5-yl]-3-pyridyl]-5-methyl-3-phenyl-isoxazole-4-carboxamide

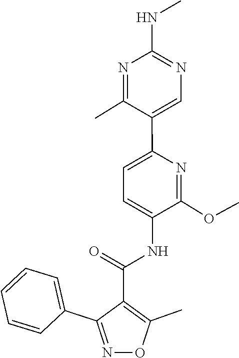 [SMILES: CNc1ncc(-c2ccc(NC(=O)c3c(-c4ccccc4)noc3C)c(OC)n2)c(C)n1]

### Step 1

To a 10 mL microwave vial was charged with 5-bromo-2-chloro-4-methyl-pyrimidine (0.42 g, 2.0 mmol), THF (2 mL), Et₃N (0.84 mL, 6.0 mmol), methylamine hydrochloride (0.27 g, 4.0 mmol) and water (0.2 mL). The vial was sealed and heated at 85° C. for 17 h. The volatiles were removed under reduced pressure and the residue was adsorbed onto silica and purified by flash column chromatography (12 g silica, elution with a 0-20% EtOAc/PE gradient) to afford 5-bromo-N,4-dimethyl-pyrimidin-2-amine (0.33 g, 78% yield) as a colourless solid. ¹H NMR (500 MHz, Chloroform-d) δ8.21 (s, 1H), 5.06 (s, 1H), 2.97 (d, J=5.1 Hz, 3H), 2.43 (s, 3H). m/z 202.0/204.0 [M+H]⁺.

### Step 2

A 10 mL microwave vial was charged with Intermediate 7 (0.20 g, 0.46 mmol), 5-bromo-N,4-dimethyl-pyrimidin-2-amine (0.11 g, 0.55 mmol), KF (80 mg, 1.4 mmol), 1,4-dioxane (4 mL) and water (1 mL). The mixture was degassed for 10 min before adding Pd-118 (0.03 g, 0.05 mmol) and the vial was sealed and stirred at room temperature for 2 h. Water (10 mL) was added and the mixture was extracted with DCM (3×10 mL). The combined organic extracts were dried over MgSO₄, filtered and concentrated under reduced pressure. The residue was adsorbed onto silica and purified by flash column chromatography (12 g silica, elution with a 0-100% EtOAc/PE gradient) to afford a solid, which was further triturated in EtOAc/PE to afford N-[2-methoxy-6-[4-methyl-2-(methylamino)pyrimidin-5-yl]-3-pyridyl]-5-methyl-3-phenyl-isoxazole-4-carboxamide (109 mg, 52% yield) as a tan solid. ¹H NMR (500 MHz, Chloroform-d) δ8.68 (d, J=8.0 Hz, 1H), 8.36 (s, 1H), 7.73 (s, 1H), 7.67-7.51 (m, 5H), 6.95 (d, J=8.0 Hz, 1H), 5.08 (q, J=4.7 Hz, 1H), 3.63 (s, 3H), 3.03 (d, J=5.0 Hz, 3H), 2.83 (s, 3H), 2.48 (s, 3H). m/z 431.1 [M+H]⁺.

### Example 193 N-[2-Methoxy-6-(2-methoxy-4,6-dimethyl-pyrimidin-5-yl)-3-pyridyl]-5-methyl-3-phenyl-isoxazole-4-carboxamide

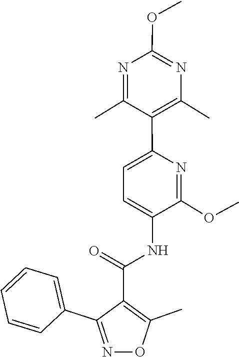 [SMILES: COc1nc(C)c(-c2ccc(NC(=O)c3c(-c4ccccc4)noc3C)c(OC)n2)c(C)n1]

### Step 1

To an ice-cooled solution of MeOH (7.5 mL) was added in portions sodium (69 mg, 3.0 mmol). The mixture was stirred until all the sodium metal has fully dissolved. 5-Bromo-2-chloro-4,6-dimethyl-pyrimidine (0.33 g, 1.5 mmol) was added to the solution and the reaction mixture was stirred for 30 min. The solvent was removed under reduced pressure and water (20 mL) was added to the residue. The solution was extracted with EtOAc (3×20 mL) and the organic extracts were dried over MgSO₄, filtered and concentrated under reduced pressure to afford 5-bromo-2-methoxy-4,6-dimethyl-pyrimidine (0.30 g, 88% yield) as an orange solid, which was used in the next step without further purification. ¹H NMR (500 MHZ, Chloroform-d) δ3.96 (s, 3H), 2.56 (s, 6H). m/z 216.9/219.0 [M+H]⁺.

### Step 2

A 10 mL microwave vial was charged with Intermediate 7 (0.20 g, 0.46 mmol), 5-bromo-2-methoxy-4,6-dimethyl-pyrimidine (0.11 g, 0.51 mmol), KF (80 mg, 1.4 mmol), 1,4-dioxane (4 mL) and water (1 mL). The mixture was degassed for 10 min before adding Pd-118 (0.03 g, 0.05 mmol) and the vial was sealed and stirred at room temperature for 14 h. Water (20 mL) was added and the mixture was extracted with DCM (3×10 mL). The combined organic extracts were dried over MgSO₄, filtered and concentrated under reduced pressure. The residue was adsorbed onto silica and purified by flash column chromatography (12 g silica, elution with a 0-50% EtOAc/PE gradient) to afford a solid, which was recrystallised from EtOAc/PE and left in the fridge overnight. The supernatant was removed to and the solid was dried to afford N-[2-methoxy-6-(2-methoxy-4,6-dimethyl-pyrimidin-5-yl)-3-pyridyl]-5-methyl-3-phenyl-isoxazole-4-carboxamide (0.12 g, 57% yield) as an orange solid. ¹H NMR (500 MHZ, Chloroform-d) δ8.72 (d, J=7.8 Hz, 1H), 7.79 (s, 1H), 7.72-7.51 (m, 5H), 6.81 (d, J=7.9 Hz, 1H), 4.00 (s, 3H), 3.59 (s, 3H), 2.85 (s, 3H), 2.24 (s, 6H). m/z 446.1 [M+H]⁺.

### Example 194 N-(6-(4,6-Dimethyl-2-(methylamino)pyrimidin-5-yl)-2-methoxypyridin-3-yl)-5-methyl-3-phenylisoxazole-4-carboxamide

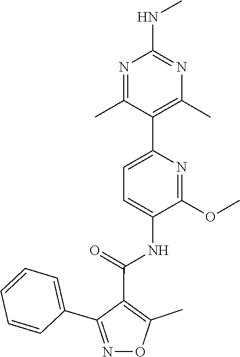 [SMILES: CNc1nc(C)c(-c2ccc(NC(=O)c3c(-c4ccccc4)noc3C)c(OC)n2)c(C)n1]

### Step 1

To a 10 mL microwave vial was charged with 5-bromo-2-chloro-4,6-dimethyl-pyrimidine (0.33 g, 1.5 mmol), THF (1.5 mL), Et₃N (0.63 mL, 4.5 mmol), methylamine hydrochloride (0.20 g, 3.0 mmol) and water (0.15 mL). The vial was sealed and heated at 85° C. for 16 h.

The volatiles were removed under reduced pressure and the residue was adsorbed onto silica and purified by flash column chromatography (12 g silica, elution with a 0-50% EtOAc/PE gradient) to afford 5-bromo-N,4,6-trimethyl-pyrimidin-2-amine (0.20 g, 59% yield) as a yellow solid. ¹H NMR (500 MHZ, Chloroform-d) δ4.91 (s, 1H), 2.96 (d, J=5.1 Hz, 3H), 2.45 (s, 6H). m/z 216.0/218.0 [M+H]⁺.

### Step 2

A 10 mL microwave vial was charged with Intermediate 7 (0.20 g, 0.46 mmol), 5-bromo-N,4,6-trimethyl-pyrimidin-2-amine (0.11 g, 0.51 mmol), KF (80 mg, 1.4 mmol), 1,4-dioxane (4 mL) and water (1 mL). The mixture was degassed for 10 min before adding Pd-118 (0.03 g, 0.05 mmol) and the vial was sealed and stirred at room temperature for 14 h. Water (20 mL) was added and the mixture was extracted with DCM (3×10 mL). The combined organic extracts were dried over MgSO₄, filtered and concentrated under reduced pressure. The residue was adsorbed onto silica and purified by flash column chromatography (12 g silica, elution with a 0-100% EtOAc/PE gradient) to afford a solid, which was triturated with EtOAc/PE to afford N-[6-[4,6-dimethyl-2-(methylamino)pyrimidin-5-yl]-2-methoxy-3-pyridyl]-5-methyl-3-phenyl-isoxazole-4-carboxamide (0.11 g, 53% yield) as an orange solid. ¹H NMR (500 MHz, Chloroform-d) δ 8.67 (d, J=8.0 Hz, 1H), 7.77 (s, 1H), 7.68-7.54 (m, 5H), 6.79 (d, J=7.9 Hz, 1H), 4.93 (q, J=4.9 Hz, 1H), 3.59 (s, 3H), 3.01 (d, J=5.0 Hz, 3H), 2.84 (s, 3H), 2.14 (s, 6H). m/z 445.1 [M+H]⁺.

### Example 195 N-(2-Methoxy-6-(7-methyl-5,6,7,8-tetrahydro-[1,2,4]triazolo[4,3-a]pyrazin-3-yl)pyridin-3-yl)-5-methyl-3-phenylisoxazole-4-carboxamide

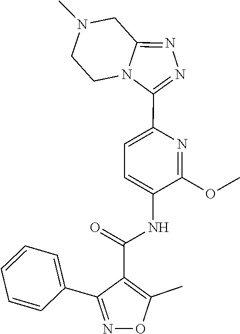 [SMILES: COc1nc(-c2nnc3n2CCN(C)C3)ccc1NC(=O)c1c(-c2ccccc2)noc1C]

### Step 1

A 20 mL microwave vial was charged with Intermediate 7 (500 mg, 1.15 mmol), tert-butyl 3-bromo-6,8-dihydro-5H-[1,2,4]triazolo[4,3-a]pyrazine-7-carboxylate (453 mg, 1.49 mmol), tripotassium phosphate (732 mg, 3.45 mmol), 1,4-dioxane (10 mL) and water (2.5 mL). The reaction mixture was degassed with nitrogen for 5 mins. Pd(dppf)Cl₂ (complexed with DCM, 0.11 g, 0.14 mmol) was quickly added and the mixture further degassed with nitrogen before stirring at 80° C. for 4 h. The reaction mixture was then concentrated under reduced pressure.

The dark residue dissolved in DCM (20 mL) and washed with water (2×10 mL). The organic layer was dried over MgSO₄, filtered and concentrated under reduced pressure. The residue was purified by flash column chromatography (12 g, silica, 0-100% EtOAc/PE+1% Et₃N over 40 CV's). CV's containing product were combined and evaporated under reduced pressure. The residue was then triturated with hot EtOAc (3 mL) and the precipitate dried under vacuum at 40° C. to afford tert-butyl 3-(6-methoxy-5-(5-methyl-3-phenylisoxazole-4-carboxamido)pyridin-2-yl)-5,6-dihydro[1,2,4]triazolo[4,3-a]pyrazine-7 (8H)-carboxylate (280.5 mg, 44%) as an off-white solid. ¹H NMR (500 MHZ, Chloroform-d) δ8.81 (d, J=8.2 Hz, 1H), 7.91 (d, J=8.2 Hz, 1H), 7.76 (br s, 1H), 7.62-7.67 (m, 3H), 7.57-7.61 (m, 2H), 4.89 (s, 2H), 4.49 (t, J=5.6 Hz, 2H), 3.84 (t, J=5.6 Hz, 2H), 3.63 (s, 3H), 2.84 (s, 3H), 1.50 (s, 9H). m/z 532.1 [M+H]⁺.

### Step 2

A 10 mL microwave vial was charged with tert-butyl 3-(6-methoxy-5-(5-methyl-3-phenylisoxazole-4-carboxamido)pyridin-2-yl)-5,6-dihydro[1,2,4]triazolo[4,3-a]pyrazine-7 (8H)-carboxylate (0.14 g, 0.25 mmol), paraformaldehyde (30 mg, 1.0 mmol) and formic acid (3 mL). The reaction mixture was heated to 95° C. and stirred for 2 h. The reaction mixture was cooled, diluted with DCM (15 mL) and washed sequentially with 2 M NaOH (2×15 mL), water (2×15 mL) and brine (1×15 mL). The organic layer was dried over MgSO₄, filtered and concentrated under reduced pressure. The residue was purified by flash column chromatography (4 g, silica, 0-10% MeOH/DCM over 25 CV's). CV's containing product were combined and evaporated under reduced pressure. The residue was then triturated with hot EtOAc (3 mL) and the precipitate dried under vacuum at 40° C. to afford N-(2-methoxy-6-(7-methyl-5,6,7,8-tetrahydro-[1,2,4]triazolo[4,3-a]pyrazin-3-yl)pyridin-3-yl)-5-methyl-3-phenylisoxazole-4-carboxamide (38 mg, 32% yield) as a cream solid. ¹H NMR (500 MHz, Chloroform-d) δ8.79 (d, J=8.3 Hz, 1H), 7.91 (d, J=8.3 Hz, 1H), 7.75 (br s, 1H), 7.55-7.67 (m, 5H), 4.48 (t, J=5.6 Hz, 2H), 3.83 (s, 2H), 3.62 (s, 3H), 2.79-2.87 (m, 5H) 2.52 (s, 3H). m/z 446.1 [M+H]⁺.

### Example 196 N-(2-Methoxy-6-(5,6,7,8-tetrahydro-[1,2,4]triazolo[4,3-a]pyrazin-3-yl)pyridin-3-yl)-5-methyl-3-phenylisoxazole-4-carboxamide dihydrochloride

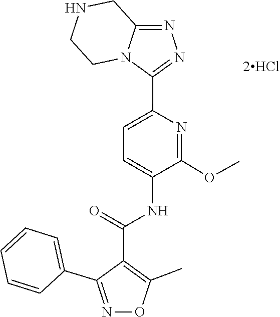 [SMILES: COc1nc(-c2nnc3n2CCNC3)ccc1NC(=O)c1c(-c2ccccc2)noc1C]

A 10 mL microwave vial was charged with tert-butyl 3-(6-methoxy-5-(5-methyl-3-phenylisoxazole-4-carboxamido)pyridin-2-yl)-5,6-dihydro[1,2,4]triazolo[4,3-a]pyrazine-7 (8H)-carboxylate (0.13 g, 0.25 mmol) (synthesised according to Example 195), HCl (2 M in ether, 1.25 mL, 2.50 mmol) and DCM (5 mL). The reaction mixture was stirred at rt overnight. The reaction mixture was concentrated under reduced pressure and the precipitate dried under vacuum at 40° C. to afford N-(2-methoxy-6-(5,6,7,8-tetrahydro-[1,2,4]triazolo[4,3-a]pyrazin-3-yl)pyridin-3-yl)-5-methyl-3-phenylisoxazole-4-carboxamide dihydrochloride (0.13 g, 98% yield) as a light yellow solid. ¹H NMR (500 MHZ, DMSO-d₆) δ9.95 (br s, 2H), 9.61 (s, 1H), 8.48 (br s, 1H), 7.84 (d, J=8.1 Hz, 2H), 7.71 (d, J=7.0 Hz, 2H), 7.51-7.60 (m, 3H), 4.75 (t, J=5.8 Hz, 2H), 4.61 (s, 2H), 3.89 (s, 3H), 3.63 (t, J=6.4 Hz, 2H), 2.67 (s, 3H). m/z 432.1 [M+H]⁺.

### Example 197 N-[4-(2-Aminopyrimidin-5-yl)-2-methoxy-phenyl]-5-methyl-3-phenyl-isoxazole-4-carboxamide hydrochloride

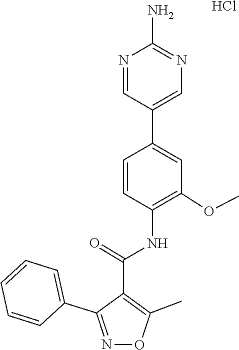 [SMILES: Cl ; COc1cc(-c2cnc(N)nc2)ccc1NC(=O)c1c(-c2ccccc2)noc1C]

### Step 1

To a microwave vial was added Intermediate 5 (1.50 g, 3.87 mmol), B₂Pin₂ (1.47 g, 5.81 mmol) and 1,4-dioxane (30 mL). The solution was degassed with nitrogen for 15 min before Pd(dppf)Cl₂ (complexed with DCM, 0.32 g, 0.39 mmol) and KOAc (11.4 g, 11.6 mmol) were quickly added. The mixture was again degassed with nitrogen for 15 min before being heated to 80° C. for 1 h using an aluminium heating block. The reaction mixture was allowed to cool and was diluted with EtOAc (30 mL). The solution was washed with water (30 mL), dried over MgSO₄, filtered and concentrated under reduced pressure to give the crude product which was purified by trituration with PE. The suspension was left to settle, the supernatant PE was removed and the trituration repeated with more PE. The resulting brown powder was dried under reduced pressure to give N-[2-methoxy-4-(4,4,5,5-tetramethyl-1,3,2-dioxaborolan-2-yl)phenyl]-5-methyl-3-phenyl-isoxazole-4-carboxamide (1.6 g, 78% yield) as a brown powder. ¹H NMR (500 MHZ, Chloroform-d) δ8.46 (d, J=8.0 Hz, 1H), 7.94 (s, 1H), 7.63 (d, J=7.3 Hz, 2H), 7.59-7.49 (m, 3H), 7.42 (d, J=8.0 Hz, 1H), 7.12 (s, 1H), 3.47 (s, 3H), 2.81 (s, 3H), 1.32 (s, 12H). m/z 435.1 [M+H]⁺.

### Step 2

A solution of KF (77 mg, 1.3 mmol), 2-amino-5-bromopyrimidine (80 μL, 0.44 mmol) and N-[2-methoxy-4-(4,4,5,5-tetramethyl-1,3,2-dioxaborolan-2-yl)phenyl]-5-methyl-3-phenyl-isoxazole-4-carboxamide (0.20 g, 0.44 mmol) in 1,4-dioxane (4 mL) and water (1 mL) was degassed with nitrogen for 15 min. Pd-118 (28 mg, 0.040 mmol) was quickly added, the solution degassed again for 10 min and the mixture left to stir at room temperature overnight.

Then, water (10 mL) was added and the reaction mixture extracted with DCM (3×10 mL). The combined organics were washed with brine (10 mL), dried over MgSO₄ and filtered before concentration to dryness. Purification by flash silica column chromatography on an ISCO system (elution with 8% MeOH/DCM gradient) yielded the free base that was subsequently treated with 1.2 eq. of HCl to give N-[4-(2-aminopyrimidin-5-yl)-2-methoxy-phenyl]-5-methyl-3-phenyl-isoxazole-4-carboxamide hydrochloride (0.10 g, 49% yield) as brownish solid. ¹H NMR (400 MHZ, DMSO-d₆) δ9.28 (s, 1H), 8.74 (s, 2H), 7.99 (d, J=8.4 Hz, 1H), 7.78-7.69 (m, 2H), 7.60-7.51 (m, 3H), 7.30 (s, 1H), 7.24 (d, J=8.2 Hz, 1H), 3.78 (s, 3H), 2.67 (s, 3H). m/z 402.1 [M+H for free base]⁺.

### Example 198 N-[4-(5-Aminopyrazin-2-yl)-2-methoxy-phenyl]-5-methyl-3-phenyl-isoxazole-4-carboxamide hydrochloride

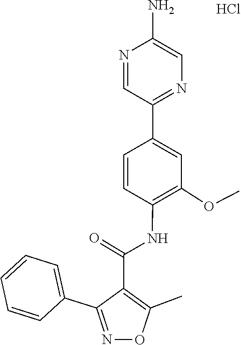 [SMILES: COc1cc(-c2cnc(N)cn2)ccc1NC(=O)c1c(-c2ccccc2)noc1C ; Cl]

A solution of KF (100 mg, 1.73 mmol), 5-bromopyrazin-2-amine (0.12 mL, 0.69 mmol) and N-[2-methoxy-4-(4,4,5,5-tetramethyl-1,3,2-dioxaborolan-2-yl)phenyl]-5-methyl-3-phenyl-isoxazole-4-carboxamide (0.25 g, 0.58 mmol) (synthesised as described in Example 197) in 1,4-dioxane (5 mL) and water (1.25 mL) was degassed with nitrogen for 15 min. Pd-118 (37 mg, 0.060 mmol) was quickly added, the solution degassed again for 10 min and the mixture left to stir at room temperature overnight. Then, water (10 mL) was added and the reaction mixture extracted with DCM (3×10 mL). The combined organics were washed with brine (10 mL), dried over MgSO₄ and filtered before concentration to dryness. Purification by flash silica column chromatography on an ISCO system (elution with 8% MeOH/DCM gradient) yielded the free base that was subsequently treated with 1.2 eq. of HCl to give N-[4-(5-aminopyrazin-2-yl)-2-methoxy-phenyl]-5-methyl-3-phenyl-isoxazole-4-carboxamide hydrochloride (51 mg, 19% yield) as brownish solid. ¹H NMR (400 MHZ, DMSO-d₆) δ9.25 (s, 1H), 8.55 (s, 1H), 8.21 (s, 1H), 8.03 (d, J=8.2 Hz, 1H), 7.77-7.68 (m, 2H), 7.55 (m, 5H), 3.76 (s, 3H), 2.67 (s, 3H). m/z 402.1 [M+H for free base]⁺.

### Example 199 N-[6-[6-(Aminomethyl)-3-pyridyl]-2-methoxy-3-pyridyl]-5-methyl-3-phenyl-isoxazole-4-carboxamide hydrochloride

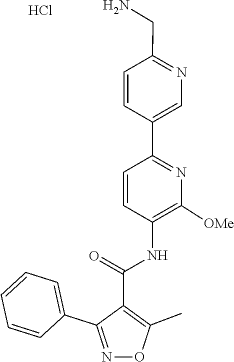 [SMILES: COc1nc(-c2ccc(CN)nc2)ccc1NC(=O)c1c(-c2ccccc2)noc1C ; Cl]

A solution of KF (95.1 mg, 1.64 mmol), Intermediate 7 (250 mg, 0.550 mmol) and (5-bromopyridin-2-ylmethyl) carbamic acid tert-butyl ester (156 mg, 0.550 mmol) in 1,4-dioxane (5.0 mL) and water (1.25 mL) was degassed for 10 min. Pd-118 (35 mg, 0.050 mmol) was added quickly, the microwave vial capped and the mixture degassed for a further 10 min. The mixture was allowed to stir at room temperature overnight. The reaction mixture was diluted with DCM (30 mL) and washed with water (20 mL). The aqueous phase was twice back-extracted with DCM (2×15 mL), the combined organic extract dried over MgSO₄ and filtered. Silica was added directly and the mixture concentrated under reduced pressure to give the crude product adsorbed onto silica, which was purified by automated column chromatography on silica (12 g, gradient from 0-10% MeOH/DCM over 25 CV) to give the Boc-protected product as a white solid (217 mg, m/z 516.1 [M+H]⁺). The Boc protected product was dissolved in a minimum amount of DCM (5.0 mL) and 4 M HCl in 1,4-dioxane was added drop-wise (0.2 mL). The mixture was stirred at room temperature and after 5 h additional 4 M HCl in 1,4-dioxane was added (0.2 mL). A further portion of 4 M HCl in 1,4-dioxane (0.4 mL) was added after 1 hour, along with additional DCM (2.0 mL) to aid stirring and the mixture was left to stir overnight at room temperature. The sample was concentrated under reduced pressure to give N-[6-[6-(aminomethyl)-3-pyridyl]-2-methoxy-3-pyridyl]-5-methyl-3-phenyl-isoxazole-4-carboxamide hydrochloride (200 mg, 77% yield) as a light yellow solid. ¹H NMR (500 MHZ, DMSO-d₆) δ9.54 (s, 1H), 9.29 (s, 1H), 8.64 (s, 3H), 8.54 (d, J=8.2 Hz, 1H), 8.41 (d, J=7.7 Hz, 1H), 7.76 (d, J=8.1 Hz, 1H), 7.74-7.68 (m, 3H), 7.61-7.51 (m, 3H), 4.26 (q, J=7.5 Hz, 2H), 3.93 (s, 3H), 2.68 (s, 3H). m/z 416.1 [M+H for free base]⁺.

### Example 200 N-(6-(1-(2-Aminoethyl)-1H-imidazol-4-yl)-2-methoxypyridin-3-yl)-5-methyl-3-phenylisoxazole-4-carboxamide dihydrochloride

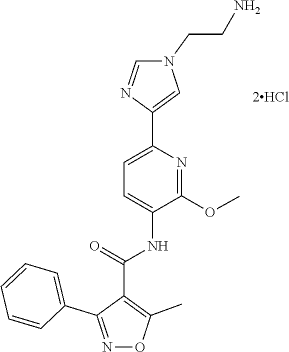 [SMILES: COc1nc(-c2cn(CCN)cn2)ccc1NC(=O)c1c(-c2ccccc2)noc1C]

### Step 1

A 20 mL microwave vial was charged with 5-bromo-1H-imidazole (515 mg, 3.50 mmol), 2-(Boc-amino)ethyl bromide (864 mg, 3.85 mmol), K₂CO₃ (1.21 g, 8.76 mmol) and MeCN (8 mL). The reaction mixture was stirred at 80° C. for 24 h. Solids were filtered and the filtrate concentrated under reduced pressure. The crude mixture was then dissolved in the minimum amount of MeOH and subjected to reverse-phase column chromatography (0-100% MeOH/water over 30 min). CV's containing product was combined to afford tert-butyl (2-(4-bromo-1H-imidazol-1-yl)ethyl) carbamate (170 mg, 10% yield) as an orange oil. ¹H NMR (500 MHZ, Chloroform-d) δ7.51 (s, 1H), 7.03 (s, 1H), 4.64 (br s, 1H), 4.11 (t, J=5.9 Hz, 2H), 3.41 (q, J=5.8 Hz, 2H), 1.44 (s, 9H). ¹³C (125 MHZ, Chloroform-d) δ28.5, 40.1, 45.6, 80.3, 102.9, 130.2, 138.6, 155.9. m/z 291.9/293.9 [M+H]⁺. The regiochemistry was confirmed by HMBC experiment on both this compound and the other regioisomer which was also isolated during the purification.

### Step 2

A 20 mL microwave vial was charged with Intermediate 7 (0.35 g, 0.80 mmol), tert-butyl (3-(4-bromo-1H-imidazol-1-yl) propyl) carbamate (0.27 g, 0.92 mmol), KF (140 mg, 2.41 mmol), 1,4-dioxane (9 mL) and water (3 mL). The reaction mixture was degassed with nitrogen for 5 mins. Pd-118 (52 mg, 0.080 mmol) was quickly added and the mixture further degassed with nitrogen before stirring at 50° C. overnight. The reaction mixture was then concentrated under reduced pressure. The dark residue dissolved in DCM (20 mL) and washed with water (2×10 mL). The organic layer was dried over MgSO₄, filtered and concentrated under reduced pressure. The residue was purified by flash column chromatography (12 g, silica, 0-10% MeOH/DCM+1% Et₃N over 25 CV's). CV's containing product were combined and evaporated under reduced pressure. The residue was then triturated with hot EtOAc (3 mL) and the precipitate dried under vacuum at 40° C. to afford tert-butyl (2-(4-(6-methoxy-5-(5-methyl-3-phenylisoxazole-4-carboxamido)pyridin-2-yl)-1H-imidazol-1-yl)ethyl) carbamate (50 mg, 11% yield) as a cream solid. ¹H NMR (500 MHZ, Chloroform-d) δ8.68 (d, J=8.2 Hz, 1H), 7.71 (br s, 1H), 7.59-7.66 (m, 3H), 7.54-7.59 (m, 2H), 7.52 (d, J=8.1 Hz, 1H), 7.46 (s, 1H), 7.41 (s, 1H), 4.64 (br s, 1H), 4.05-4.16 (m, 2H), 3.65 (s, 3H), 3.46 (q, J=6.0 Hz, 2H), 2.83 (s, 3H), 1.43 (s, 9H). m/z 519.2 [M+H]⁺.

### Step 3

A 10 mL microwave vial was charged with tert-butyl (2-(4-(6-methoxy-5-(5-methyl-3-phenylisoxazole-4-carboxamido)pyridin-2-yl)-1H-imidazol-1-yl)ethyl) carbamate (50 mg, 0.10 mmol), HCl (2M in ether, 0.71 mL, 1.4 mmol) and DCM (3 mL). The reaction mixture was stirred at rt overnight. After concentrating the reaction mixture under reduced pressure, the precipitate was dissolved in EtOAc (20 mL) and washed with saturated NaHCO₃ (3×10 mL). The organic layer was dried over MgSO₄, filtered and concentrated under reduced pressure. The precipitate was re-dissolved in DCM (1 mL) and HCl (2 M in ether, 74 μL, 0.15 mmol) was then added. The reaction mixture was stirred at room temperature for 5 mins. The mixture was then concentrated under reduced pressure to afford N-(6-(1-(2-aminoethyl)-1H-imidazol-4-yl)-2-methoxypyridin-3-yl)-5-methyl-3-phenylisoxazole-4-carboxamide dihydrochloride (8 mg, 16% yield) as a light yellow solid. ¹H NMR (500 MHZ, DMSO-d₆) δ9.49 (br s, 1H), 8.35 (br s, 1H), 7.98-8.13 (m, 4H), 7.71 (d, J=7.2 Hz, 2H), 7.49-7.60 (m, 5H), 4.39 (br s, 2H), 3.91 (s, 3H), 2.66 (s, 3H). One CH₂ signal overlapping with NMR solvent. m/z 419.1 [M+H for free base]⁺.

### Example 201 N-[6-[2-(1-Hydroxy-1-methyl-ethyl)pyrimidin-5-yl]-2-methoxy-3-pyridyl]-5-methyl-3-phenyl-isoxazole-4-carboxamide

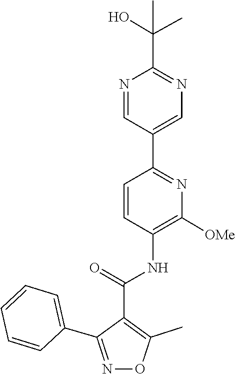 [SMILES: COc1nc(-c2cnc(C(C)(C)O)nc2)ccc1NC(=O)c1c(-c2ccccc2)noc1C]

### Step 1

To a dry 100 mL RBF equipped with a magnetic stirrer bar was added 5-bromo-2-iodopyrimidine (0.50 g, 1.76 mmol) and anhydrous THF (10.0 mL). The solution was cooled to −78° C. using a dry ice-acetone bath. After allowing the reaction mixture to cool, n-BuLi (1.2 M, 1.46 mL, 1.76 mmol) was added slowly drop-wise with rapid stirring of the reaction mixture. Acetone (0.300 mL) was added drop-wise immediately following completion of addition of n-BuLi. Upon completion of addition, the cooling bath was removed and the mixture allowed to warm up to room temperature gradually over 1 h. The mixture was quenched with water (10 mL) and poured into a separating funnel. The resulting aqueous phase was extracted with DCM (4×50 mL). The combined organic extract was dried over MgSO₄, filtered and concentrated under reduced pressure to give the crude product, which was purified by automated column chromatography on silica (12 g, gradient from 0-10% MeOH/DCM over 25 CV) to give 2-(5-bromopyrimidin-2-yl) propan-2-ol (84.0 mg, 21% yield) as a light orange crystalline solid. ¹H NMR (500 MHZ, Chloroform-d) δ8.66 (s, 2H), 1.62 (s, 6H). m/z 219.0 [M+H for ⁸¹Br]⁺.

### Step 2

A solution of KF (26 mg, 0.45 mmol), Intermediate 7 (68 mg, 0.15 mmol), 2-(5-bromopyrimidin-2-yl) propan-2-ol (34 mg, 0.15 mmol), 1,4-dioxane (2 mL) and water (0.5 mL) in a 20 mL Biotage microwave vial was degassed for 10 min. Pd-118 (10 mg, 0.010 mmol) was added quickly, the microwave vial capped and the mixture degassed for a further 10 min. The mixture was allowed to stir at room temperature overnight. The reaction mixture was diluted with DCM (30 mL) and washed with water (20 mL). The aqueous phase was twice back-extracted with DCM (2×15 mL), the combined organic extract dried over MgSO₄, filtered and concentrated under reduced pressure to give the crude product, which was purified by automated column chromatography on silica (12 g, 0-10% MeOH/DCM gradient over 25 CV). After evaporation, the resultant solid was triturated with PE (3×5 mL), with the supernatant being removed each time after allowing the solid to settle. The resulting solid was further dried using a Biotage V10 to give N-[6-[2-(1-hydroxy-1-methyl-ethyl)pyrimidin-5-yl]-2-methoxy-3-pyridyl]-5-methyl-3-phenyl-isoxazole-4-carboxamide (28 mg, 40% yield) as a beige solid. ¹H NMR (500 MHZ, Chloroform-d) δ8.95 (s, 2H), 8.81 (d, J=8.2 Hz, 1H), 8.10 (d, J=8.3 Hz, 1H), 7.88 (s, 1H), 7.67-7.60 (m, 3H), 7.59-7.54 (m, 2H), 3.77 (s, 3H), 2.84 (s, 3H), 1.90 (s, 1H), 1.64 (s, 6H). m/z 446.1 [M+H]⁺.

### Example 202 N-(6-(2-(Aminomethyl)pyrimidin-5-yl)-2-methoxypyridin-3-yl)-1-methyl-4-(pyridin-2-yl)-1H-1,2,3-triazole-5-carboxamide hydrochloride

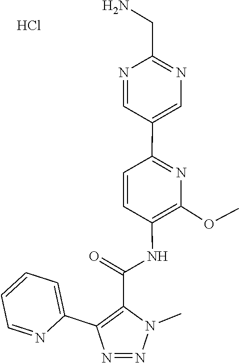 [SMILES: COc1nc(-c2cnc(CN)nc2)ccc1NC(=O)c1c(-c2ccccn2)nnn1C ; Cl]

As described for the synthesis of Example 180, substituting N-(6-bromo-2-methoxypyridin-3-yl)-1-methyl-4-(pyridine-2-yl)-1H-1,2,3-triazole-5-carboxamide (synthesised using appropriate starting materials by the method described in Step 1 of Example 154) for N-(6-bromo-2-methoxypyridin-3-yl)-1-methyl-4-phenyl-1H-1,2,3-triazole-5-carboxamide. At the final step, after concentrating the reaction mixture under reduced pressure, 1:1 MeOH:NaHCO₃ (10 mL) was added to the precipitate which was then filtered and washed with additional MeOH (5 mL) and H₂O (5 mL). The filtered solid was then re-dissolved in DCM (5 mL) and 2 M HCl in ether (30 μL, 0.18 mmol) was then added. The reaction mixture was stirred at room temperature for 5 mins. The mixture was then concentrated under reduced pressure to afford N-(6-(2-(aminomethyl)pyrimidin-5-yl)-2-methoxypyridin-3-yl)-1-methyl-4-(pyridin-2-yl)-1H-1,2,3-triazole-5-carboxamide hydrochloride (10 mg, 12% yield) as a yellow solid. ¹H NMR (500 MHz, DMSO-d₆) δ14.45 (s, 1H), 9.54 (s, 2H), 8.85 (d, J=8.1 Hz, 1H), 8.78 (d, J=4.9 Hz, 1H), 8.49 (br s, 3H), 8.41 (d, J=8.1 Hz, 1H), 8.16 (t, J=7.8 Hz, 1H), 7.91 (d, J=8.1 Hz, 1H), 7.66 (t, J=6.4 Hz, 1H), 4.34-4.47 (m, 5H), 4.25 (s, 3H). m/z 418.1 [M+H]⁺.

### Example 203 N-(6-(2-(Aminomethyl)pyrimidine-5-yl)-2-methoxypyridin-3-yl)-4-(5-chloropyridin-2-yl)-1-methyl-1H-1,2,3-triazole-5-carboxamide hydrochloride

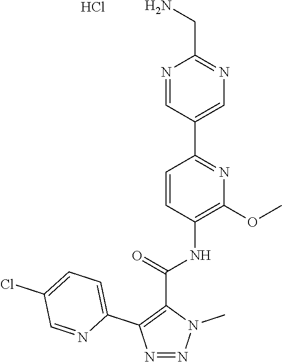 [SMILES: COc1nc(-c2cnc(CN)nc2)ccc1NC(=O)c1c(-c2ccc(Cl)cn2)nnn1C ; Cl]

As described for the synthesis of Example 180, substituting N-(6-bromo-2-methoxypyridin-3-yl)-4-(5-chloropyridine-2-yl)-1-methyl-1H-1,2,3-triazole-5-carboxamide (synthesised using appropriate starting materials by the method described in Step 1 of Example 154) for N-(6-bromo-2-methoxypyridin-3-yl)-1-methyl-4-phenyl-1H-1,2,3-triazole-5-carboxamide to give N-(6-(2-(aminomethyl)pyrimidine-5-yl)-2-methoxypyridin-3-yl)-4-(5-chloropyridin-2-yl)-1-methyl-1H-1,2,3-triazole-5-carboxamide hydrochloride (45 mg, 94% yield) as a light yellow solid. ¹H NMR (500 MHZ, DMSO-d₆) δ13.54 (s, 1H), 9.54 (s, 2H), 8.80 (d, J=8.1 Hz, 1H), 8.69-8.74 (m, 1H), 8.48 (br s, 3H), 8.38 (dd, J=8.8 & 2.5 Hz, 1H), 8.28 (d, J=8.5 Hz, 1H), 7.91 (d, J=7.7 Hz, 1H), 4.39 (br s, 5H), 4.23 (s, 3H). m/z 452.0/454.0 [M+H]⁺.

### Example 204 3-(4-Fluorophenyl)-N-(6-imidazol-1-yl-2-isopropoxy-3-pyridyl)-5-methyl-isoxazole-4-carboxamide

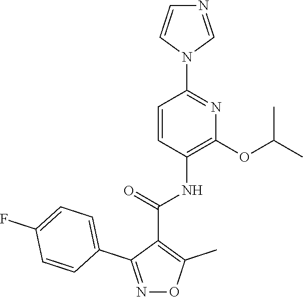 [SMILES: Cc1onc(-c2ccc(F)cc2)c1C(=O)Nc1ccc(-n2ccnc2)nc1OC(C)C]

### Step 1

A microwave vial was charged with a suspension of sodium hydride (60% in mineral oil, 700 mg, 17.5 mmol) in dry THF (3 mL). The mixture was cooled to 0° C. in an ice-water bath and propan-2-ol (3.98 mL, 51.6 mmol) was added dropwise. After stirring at 0° C. for 20 min, the mixture was allowed to reach room temperature and stirring was continued for 10 more mins. Then, 2,6-dibromopyridin-3-amine (500 mg, 1.98 mmol) in THF (2 mL) was added and the reaction mixture was heated at 80° C. for 6 h and at 65° C. for 10 h. The crude mixture was quenched with saturated NH₄Cl (10 mL). THF was removed under reduced pressure and DCM (10 mL) was added to the resulting aqueous suspension. The organic layer was separated, and the aqueous phase was extracted with DCM (2×10 mL)). The combined organic extracts were washed with brine (10 mL), dried over MgSO₄, filtered, concentrated under reduced pressure and passed through an SCX-2 column. The collected organic was subsequently concentrated under reduced pressure to give 6-bromo-2-isopropoxy-pyridin-3-amine (390 mg, 81% yield) as a brown oil. The crude product was used for the next step without further purification. ¹H NMR (400 MHZ, DMSO-d₆) δ6.88-6.79 (m, 2H), 5.11 (heptet, J=6.2 Hz, 1H), 5.01 (s, 2H), 1.29 (d, J=6.2 Hz, 6H). m/z 231.0-233.0 [M+H]⁺.

### Step 2

To a mixture of 6-bromo-2-isopropoxy-pyridin-3-amine (1.20 g, 5.19 mmol) DIPEA (1.81 mL, 10.4 mmol) in DCM (40 mL) was added dropwise a 0.4 M solution of 3-(4-fluorophenyl)-5-methyl-isoxazole-4-carbonyl chloride (13.0 mL, 5.19 mmol) in DCM (synthesised using appropriate starting materials by the method described in Step 1 of Example 1). The reaction mixture was stirred overnight at rt. Then, water (30 mL) was added and the reaction mixture extracted with DCM (3×30 mL). The combined organics were washed with brine (30 mL), dried over MgSO₄ and filtered before concentration to dryness. Purification by flash silica column chromatography on an ISCO system (elution with 20% EtOAc/PE gradient) gave N-(6-bromo-2-isopropoxy-3-pyridyl)-3-(4-fluorophenyl)-5-methyl-isoxazole-4-carboxamide (2.00 g, 85% yield) as a brown solid. ¹H NMR (400 MHz, DMSO-d₆) δ9.47 (s, 1H), 8.20 (d, J=8.1 Hz, 1H), 7.77 (dd, J=8.5, 5.6 Hz, 2H), 7.37 (t, J=8.7 Hz, 2H), 7.22 (d, J=8.1 Hz, 1H), 5.15 (heptet, J=6.2 Hz, 1H), 2.66 (s, 3H), 1.22 (d, J=6.2 Hz, 6H). m/z 434.0/436.0 [M+H]⁺.

### Step 3

A 10 mL microwave vial was charged with N-(6-bromo-2-isopropoxy-3-pyridyl)-3-(4-fluorophenyl)-5-methyl-isoxazole-4-carboxamide (0.40 g, 0.92 mmol), imidazole (78 mg, 1.2 mmol), L-proline (42 mg, 0.37 mmol), K₂CO₃ (255 mg, 1.84 mmol) and DMSO (3.5 mL), and the reaction mixture was degassed. Copper (I) iodide (70 mg, 0.37 mmol) was added to the reaction and the vial was sealed and heated at 80° C. for 16 h. The reaction mixture was partitioned between EtOAc (50 mL) and water (50 mL) and the phases were separated. The aqueous layer was further extracted with EtOAc (50 mL). The combined organic extracts were washed with water (3×25 mL), saturated NH₄Cl (25 mL), brine (25 mL), dried over MgSO₄, filtered and concentrated under reduced pressure. The residue was adsorbed onto silica and purified by flash column chromatography (12 g silica, elution with a 0-5% MeOH/DCM gradient) to afford an orange foam. The crude material was repurified by flash column chromatography (12 g silica, elution with a 0-10% MeOH/EtOAc gradient) to afford an off-white foam. Precipitation was induced by small amount of EtOAc and then this was recrystallised from EtOAc/PE to afford 3-(4-fluorophenyl)-N-(6-imidazol-1-yl-2-isopropoxy-3-pyridyl)-5-methyl-isoxazole-4-carboxamide (0.11 g, 27% yield) as an orange solid. ¹H NMR (500 MHZ, Chloroform-d) δ8.83 (d, J=8.3 Hz, 1H), 8.21 (s, 1H), 7.74-7.64 (m, 2H), 7.62 (s, 1H), 7.50 (s, 1H), 7.23 (t, J=8.6 Hz, 2H), 7.16 (s, 1H), 6.89 (d, J=8.3 Hz, 1H), 5.22 (heptet, J=6.2 Hz, 1H), 2.79 (s, 3H), 1.12 (d, J=6.2 Hz, 6H). m/z 422.2 [M+H]⁺.

### Example 205 N-(6-(2-(Aminomethyl)pyrimidin-5-yl)-2-isopropoxypyridin-3-yl)-3-(4-fluorophenyl)-5-methylisoxazole-4-carboxamide hydrochloride

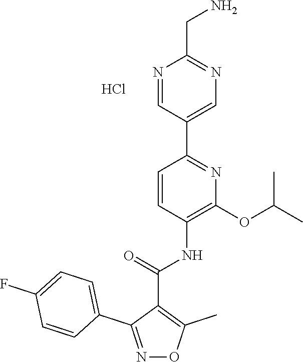 [SMILES: Cc1onc(-c2ccc(F)cc2)c1C(=O)Nc1ccc(-c2cnc(CN)nc2)nc1OC(C)C ; Cl]

### Step 1

In a 100 mL three-necked RBF was added N-(6-bromo-2-isopropoxy-3-pyridyl)-3-(4-fluorophenyl)-5-methyl-isoxazole-4-carboxamide (988 mg, 2.28 mmol) (synthesised according to Steps 1 and 2 in Example 204), bis(pinacolato)diboron (750 mg, 2.96 mmol) and KOAc (670 mg, 6.83 mmol) in 1,4-dioxane (15.6 mL). The mixture was degassed by stirring under vacuum for 25 min and then Pd(dppf)Cl₂ (complexed with DCM, 94 mg, 0.11 mmol) was added and the reaction mixture was heated at 90° C. for 1 h. The reaction mixture was allowed to cool and the solvent was removed under reduced pressure. The crude residue was dissolved in EtOAc (50 mL) and washed with water (50 mL). The layers were separated and the organic layer was dried over MgSO₄, filtered and concentrated under reduced pressure to give a crude product which was triturated with PE (3×50 mL).

The residual solvent was removed under reduced pressure to afford 3-(4-fluorophenyl)-N-[2-isopropoxy-6-(4,4,5,5-tetramethyl-1,3,2-dioxaborolan-2-yl)-3-pyridyl]-5-methyl-isoxazole-4-carboxamide (787 mg, 68% yield) as a brown solid. ¹H NMR (500 MHZ, Chloroform-d) δ8.63 (d, J=7.8 Hz, 1H), 7.80 (s, 1H), 7.70-7.64 (m, 2H), 7.44 (d, J=7.8 Hz, 1H), 7.19 (t, J=8.6 Hz, 2H), 5.44 (heptet, J=6.2 Hz, 1H), 2.77 (s, 3H), 1.34 (s, 12H), 1.03 (d, J=6.2 Hz, 6H). m/z 400.1 [M+H for boronic acid]⁺.

### Step 2

A 10 mL microwave vial was charged with a stirrer bar, 3-(4-fluorophenyl)-N-[2-isopropoxy-6-(4,4,5,5-tetramethyl-1,3,2-dioxaborolan-2-yl)-3-pyridyl]-5-methyl-isoxazole-4-carboxamide (0.20 g, 0.42 mmol), Intermediate 11 (0.12 g, 0.42 mmol) and KF (72 mg, 1.3 mmol), and the solids were dissolved in 1,4-dioxane (3.5 mL) and water (0.35 mL). The mixture was degassed before treated with Pd-118 (14 mg, 0.020 mmol) and the reaction was stirred at room temperature for 91 h. Water (20 mL) was added and the mixture was extracted with DCM (3×20 mL). The combined organic extracts were dried over MgSO₄, filtered and concentrated under reduced pressure. The residue was adsorbed onto silica and purified by flash column chromatography (12 g silica, elution with a 0-50% EtOAc/PE) to afford a solid, was triturated in ether to afford tert-butyl N-[[5-[5-[[3-(4-fluorophenyl)-5-methyl-isoxazole-4-carbonyl]amino]-6-isopropoxy-2-pyridyl]pyrimidin-2-yl]methyl]carbamate (0.11 g, 46% yield) as an off-white solid. ¹H NMR (500 MHZ, Chloroform-d) δ9.20 (s, 2H), 8.81 (d, J=8.2 Hz, 1H), 7.73 (s, 1H), 7.72-7.64 (m, 2H), 7.35 (d, J=8.2 Hz, 1H), 7.23 (t, J=8.6 Hz, 2H), 5.70 (s, 1H), 5.31 (heptet, J=6.3 Hz, 1H), 4.64 (d, J=5.1 Hz, 2H), 2.79 (s, 3H), 1.48 (s, 9H), 1.12 (d, J=6.3 Hz, 6H). m/z 563.2 [M+H]⁺.

### Step 3

To a stirring solution of tert-butyl N-[[5-[5-[[3-(4-fluorophenyl)-5-methyl-isoxazole-4-carbonyl]amino]-6-isopropoxy-2-pyridyl]pyrimidin-2-yl]methyl]carbamate (0.11 g, 0.20 mmol) in DCM (2 mL) was added HCl (4 M in 1,4-dioxane, 1 mL, 4 mmol) and the reaction mixture was left to stir at room temperature for 2.5 h. The volatiles were removed under reduced pressure and the solid was dried in a vacuum oven (40° C., 30 mbar) to afford N-[6-[2-(aminomethyl)pyrimidin-5-yl]-2-isopropoxy-3-pyridyl]-3-(4-fluorophenyl)-5-methyl-isoxazole-4-carboxamide hydrochloride (0.10 g, 98% yield) as an off-white solid. ¹H NMR (500 MHZ, DMSO-d₆) δ9.45 (s, 2H), 9.43 (s, 1H), 8.56 (s, 3H), 8.45 (d, J=8.1 Hz, 1H), 7.84-7.73 (m, 3H), 7.38 (t, J=8.8 Hz, 2H), 5.40 (heptet, J=6.2 Hz, 1H), 4.36 (s, 2H), 2.70 (s, 3H), 1.27 (d, J=6.1 Hz, 6H). m/z 463.2 [M+H for free base]⁺.

### Example 206 N-(6-(1H-Imidazol-1-yl)-2-isopropoxypyridin-3-yl)-5-methyl-3-phenylisoxazole-4-carboxamide

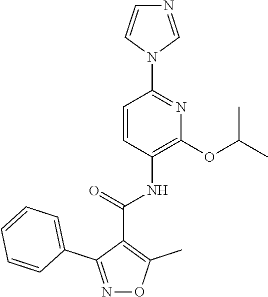 [SMILES: Cc1onc(-c2ccccc2)c1C(=O)Nc1ccc(-n2ccnc2)nc1OC(C)C]

### Step 1

To a mixture of 6-bromo-2-isopropoxy-pyridin-3-amine (1.4 g mg, 6.1 mmol) (synthesised according to Step 1 in Example 204), DIPEA (2.11 mL, 12.1 mmol) in DCM (45 mL) was added dropwise a 0.4 M solution of 5-methyl-3-phenyl-isoxazole-4-carbonyl chloride (15 mL, 6.1 mmol) (synthesised according to Step 1 in Example 1) in DCM. The reaction mixture was stirred overnight at rt. Then, water (30 mL) was added and the reaction mixture extracted with DCM (3×30 mL). The combined organics were washed with brine (30 mL), dried over MgSO₄ and filtered before concentration to dryness. Purification by flash silica column chromatography on an ISCO system (elution with 20% EtOAc/PE gradient) to yield N-(6-bromo-2-isopropoxy-3-pyridyl)-5-methyl-3-phenyl-isoxazole-4-carboxamide (2.10 g, 77% yield). ¹H NMR (400 MHZ, DMSO-d₆) δ9.49 (s, 1H), 8.17 (d, J=7.7 Hz, 1H), 7.74-7.66 (m, 2H), 7.55-7.51 (m, 3H), 7.21 (d, J=8.1 Hz, 1H), 5.15 (heptet, J=6.2 Hz, 1H), 2.66 (s, 3H), 1.22 (d, J=6.2 Hz, 6H). m/z 416.0/418.0 [M+H]⁺.

### Step 2

A 10 mL microwave vial was charged with N-(6-bromo-2-isopropoxy-3-pyridyl)-5-methyl-3-phenyl-isoxazole-4-carboxamide (0.30 g, 0.72 mmol), imidazole (54 mg, 0.79 mmol), L-proline (25 mg, 0.22 mmol), K₂CO₃ (199 mg, 1.44 mmol) and DMSO (4 mL), and the reaction mixture was degassed. Copper (I) iodide (41 mg, 0.22 mmol) was added to the reaction and the vial was sealed and heated at 80° C. for 16 h. The reaction mixture was partitioned between DCM (25 mL) and water (25 mL) and the phases were separated. The aqueous layer was further extracted with DCM (25 mL). The combined organic extracts were washed with brine (25 mL), dried over MgSO₄, filtered and concentrated under reduced pressure. The residue was adsorbed onto silica and purified by flash column chromatography (12 g silica, elution with a 0-5% MeOH/DCM gradient) to afford N-(6-imidazol-1-yl-2-isopropoxy-3-pyridyl)-5-methyl-3-phenyl-isoxazole-4-carboxamide (140 mg, 46% yield) as a light yellow solid. ¹H NMR (400 MHZ, Chloroform-d) δ8.80 (d, J=8.3 Hz, 1H), 8.19 (s, 1H), 7.70-7.63 (m, 3H), 7.59-7.47 (m, 4H), 7.14 (s, 1H), 6.87 (d, J=8.3 Hz, 1H), 5.14 (heptet, J=6.1 Hz, 1H), 2.78 (s, 3H), 1.05 (d, J=6.2 Hz, 6H). m/z 404.2 [M+H]⁺.

### Example 207 N-(6-(2-(Aminomethyl)pyrimidin-5-yl)-2-isopropoxypyridin-3-yl)-5-methyl-3-phenylisoxazole-4-carboxamide hydrochloride

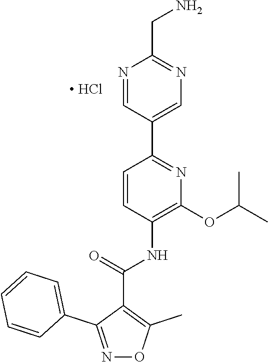 [SMILES: Cc1onc(-c2ccccc2)c1C(=O)Nc1ccc(-c2cnc(CN)nc2)nc1OC(C)C ; "· HCl"]

A 10 mL microwave vial was charged with a stirrer bar, N-[2-isopropoxy-6-(4,4,5,5-tetramethyl-1,3,2-dioxaborolan-2-yl)-3-pyridyl]-5-methyl-3-phenyl-isoxazole-4-carboxamide (0.30 g, 0.65 mmol) (synthesised using appropriate starting materials using the method described in Steps 1 and 2 in Example 204), Intermediate 11 (0.18 mL, 0.71 mmol) and KF (113 mg, 1.94 mmol) were dissolved in 1,4-dioxane (5 mL) and water (1 mL). The mixture was degassed and then treated with Pd-118 (0.02 g, 0.03 mmol) and the vial was sealed and left to stir at room temperature for 16 h. Water (20 mL) was added and the mixture was extracted with DCM (3×20 mL). The combined organic extracts were dried over MgSO₄, filtered and concentrated under reduced pressure. The crude material was then treated with HCl (4 M in 1,4-dioxane, 0.97 mL, 3.9 mmol) and once full deprotection was achieved, the reaction mixture was basified and extracted with DCM. The combined organic extracts were dried over MgSO₄, filtered and concentrated under reduced pressure. Purification by flash column chromatography (12 g, elution with a 10% MeOH/DCM gradient) afforded the free base, which was subsequently treated with 1.2 eq. of HCl to give N-[6-[2-(aminomethyl)pyrimidin-5-yl]-2-isopropoxy-3-pyridyl]-5-methyl-3-phenyl-isoxazole-4-carboxamide hydrochloride (73 mg, 22% yield) as yellow solid. ¹H NMR (500 MHZ, Methanol-d₄) δ9.40 (s, 2H), 8.67 (d, J=8.1 Hz, 1H), 7.74-7.69 (m, 2H), 7.63 (d, J=8.1 Hz, 1H), 7.60-7.52 (m, 3H), 5.39 (hept, J=6.2 Hz, 1H), 4.42 (s, 2H), 2.74 (s, 3H), 1.23 (d, J=6.2 Hz, 6H). m/z 445.2 [M+H for free base]⁺.

### Example 208 3-(4-Fluorophenyl)-N-(2-isopropoxy-6-(5,6,7,8-tetrahydroimidazo[1,2-a]pyrazin-3-yl)pyridin-3-yl)-5-methylisoxazole-4-carboxamide hydrochloride

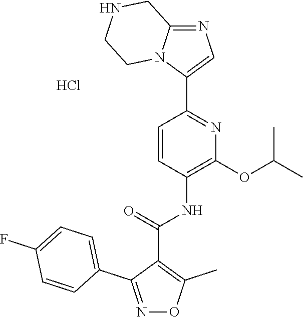 [SMILES: Cc1onc(-c2ccc(F)cc2)c1C(=O)Nc1ccc(-c2cnc3n2CCNC3)nc1OC(C)C ; Cl]

A 10 mL microwave vial was charged with a stirrer bar, 3-(4-fluorophenyl)-N-[2-isopropoxy-6-(4,4,5,5-tetramethyl-1,3,2-dioxaborolan-2-yl)-3-pyridyl]-5-methyl-isoxazole-4-carboxamide (0.30 g, 0.65 mmol) (synthesised as described in Example 205), 7-boc-3-bromo-5,6-dihydro-8H-imidazo[1,2-a]pyrazine (0.18 mL, 0.71 mmol) and KF (113 mg, 1.94 mmol), and the solids were dissolved in 1,4-dioxane (5 mL) and water (1 mL). The mixture was degassed and then treated with Pd-118 (21 mg, 0.03 mmol) and the vial was sealed and left to stir at room temperature for 16 h. Water (20 mL) was added and the mixture was extracted with DCM (3×20 mL). The combined organic extracts were dried over MgSO₄, filtered and concentrated under reduced pressure. The crude material was then treated with HCl (4 M in 1,4-dioxane, 0.97 mL, 3.88 mmol) and once full deprotection was achieved, the reaction mixture was basified and extracted with DCM. The combined organic extracts were dried over MgSO₄, filtered and concentrated under reduced pressure. Purification by flash column chromatography (12 g silica, elution with an 80% MeOH/EtOAc gradient) afforded the free base. The free base (0.02 g, 0.05 mmol) was dissolved in DCM (0.71 mL) and was treated with HCl (70 μL, 2 M in ether, 0.14 mmol) and the reaction mixture was left to stir at room temperature for 1 h. The volatiles were removed under reduced pressure and the material was dried in the vacuum oven (40° C.) to afford 3-(4-fluorophenyl)-N-(2-isopropoxy-6-(5,6,7,8-tetrahydroimidazo[1,2-a]pyrazin-3-yl)pyridin-3-yl)-5-methylisoxazole-4-carboxamide hydrochloride (23 mg, 92% yield) as a white solid. ¹H NMR (400 MHZ, DMSO-d₆) δ10.00 (s, 2H), 9.46 (s, 1H), 8.37 (d, J=7.7 Hz, 1H), 7.95 (s, 1H), 7.79 (dd, J=8.4, 5.4 Hz, 2H), 7.47 (d, J=8.1 Hz, 1H), 7.39 (t, J=8.7 Hz, 2H), 5.23 (heptet, J=6.1 Hz, 1H), 4.71 (d, J=5.9 Hz, 2H), 4.59 (s, 2H), 3.68 (t, J=5.8 Hz, 2H), 2.69 (s, 3H), 1.26 (d, J=6.1 Hz, 6H). m/z 477.1 [M+H for free base]⁺.

### Example 209 3-(4-Fluorophenyl)-N-(2-isopropoxy-6-(7-methyl-5,6,7,8-tetrahydroimidazo[1,2-a]pyrazin-3-yl)pyridin-3-yl)-5-methylisoxazole-4-carboxamide hydrochloride

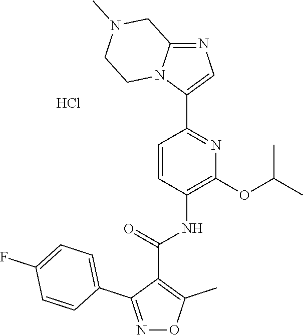 [SMILES: Cc1onc(-c2ccc(F)cc2)c1C(=O)Nc1ccc(-c2cnc3n2CCN(C)C3)nc1OC(C)C ; Cl]

To a 10 mL microwave vial was added tert-butyl 3-[6-methoxy-5-[(5-methyl-3-phenyl-isoxazole-4-carbonyl)amino]-2-pyridyl]-6,8-dihydro-5H-imidazo[1,2-a]pyrazine-7-carboxylate (50 mg, 0.10 mmol) (synthesised as crude intermediate as described in Example 208) and paraformaldehyde (13 mg, 0.42 mmol) in formic acid (2.2 mL), and the reaction was heated at 95° C. for 2 h. The reaction mixture was left to cool to room temperature and diluted with DCM, then washed sequentially with 2 M NaOH, water and brine. The organic layer was dried over MgSO₄, filtered and concentrated under reduced pressure. The residue was adsorbed onto silica and purified by flash column chromatography (24 g silica, elution with a 60% MeOH/EtOAc gradient) to afford the free base. The product was dissolved in DCM (1 mL) and was then treated with HCl (0.16 mL, 2 M in ether, 0.31 mmol) and the reaction mixture was left to stir at room temperature for 1 h. The volatiles were removed under reduced pressure and the material was dried in the vacuum oven (40° C.) to afford 3-(4-fluorophenyl)-N-(2-isopropoxy-6-(7-methyl-5,6,7,8-tetrahydroimidazo[1,2-a]pyrazin-3-yl)pyridin-3-yl)-5-methylisoxazole-4-carboxamide hydrochloride (40 mg, 69% yield) as a yellow solid. ¹H NMR (400 MHZ, DMSO-d₆) δ9.46 (s, 1H), 8.38 (d, J=8.0 Hz, 1H), 7.98 (s, 1H), 7.79 (dd, J=8.4, 5.4 Hz, 2H), 7.48 (d, J=8.1 Hz, 1H), 7.39 (t, J=8.7 Hz, 2H), 5.23 (hept, J=6.1 Hz, 1H)), 4.76 (s, 2H), 4.58 (s, 2H), 3.67 (s, 2H), 2.93 (s, 3H), 2.69 (s, 3H), 1.25 (d, J=6.1 Hz, 6H). m/z 491.2 [M+H]⁺.

### Example 210 5-(Azetidin-1-yl)-3-(4-fluorophenyl)-N-(6-isoxazol-4-yl-2-methoxy-3-pyridyl) isoxazole-4-carboxamide

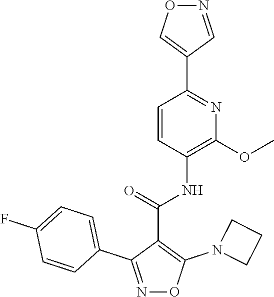 [SMILES: COc1nc(-c2cnoc2)ccc1NC(=O)c1c(-c2ccc(F)cc2)noc1N1CCC1]

### Step 1

A 500 mL RBF was charged with hydroxylamine hydrochloride (10.0 g, 144 mmol) and Na-2CO₃ (15.3 g, 144 mmol) and the solids were suspended in water (250 mL)/EtOH (50 mL). The mixture was stirred for 5 mins before treating with 4-fluorobenzaldehyde (12.9 mL, 120 mmol) over 5 min. The reaction mixture was left to stir at room temperature for 18 hours. The reaction mixture was extracted with EtOAc (3×250 mL) and the combined organic extracts were washed with brine (250 mL), dried over MgSO₄, filtered and concentrated under reduced pressure to give 4-fluorobenzaldehyde oxime (15.7 g, 89% yield) as a yellow solid. ¹H NMR (500 MHZ, Chloroform-d) δ8.14 (s, 1H), 7.64-7.54 (m, 3H), 7.10 (t, J=8.7 Hz, 2H).

### Step 2

To a stirring solution of 4-fluorobenzaldehyde oxime (15.68 g, 112.7 mmol) in dry DMF (110 mL) was added N-chlorosuccinimide (15.05 g, 112.7 mmol) in portions over 5 min. The reaction was left to stir at room temperature over the weekend. Additional N-chlorosuccinimide (1.50 g, 11.3 mmol) was added and the reaction mixture was further stirred at room temperature. The reaction was quenched with water (200 mL) and then extracted with ether (2×200 mL). The combined organic extracts were washed with water (100 mL), brine (100 mL), dried over MgSO₄ and concentrated under reduced pressure to afford 4-fluoro-N-hydroxy-benzimidoyl chloride (19.20 g, 93% yield) as an off-white solid. ¹H NMR (500 MHZ, Chloroform-d) δ7.97 (s, 1H), 7.84 (dd, J=8.9, 5.3 Hz, 2H), 7.10 (t, J=8.6 Hz, 2H).

### Step 3

In a 3-necked 500 mL RBF, a solution of 4-fluoro-N-hydroxy-benzimidoyl chloride (7.84 g, 45.2 mmol) and methyl cyanoacetate (5.18 mL, 58.7 mmol) in MeOH (200 mL) was cooled to −10° C. Upon reaching the temperature, sodium methoxide (7.32 g, 135 mmol) was added in portions over 5 mins while maintaining the temperature at <−5° C. The reaction mixture was left to stir at −10° C. for 5 min, then at rt for 2 h, during which a yellow clear solution produced precipitation. The solvent was removed under reduced pressure and the solids were suspended in water (100 mL) and filtered under vacuum. The solids were washed with additional water (50 mL) and left to dry in a vacuum oven (40° C.) o/n to afford methyl 5-amino-3-(4-fluorophenyl) isoxazole-4-carboxylate (9.10 g, 81% yield) as a yellow solid. ¹H NMR (500 MHZ, Chloroform-d) δ7.64 (dd, J=8.6, 5.5 Hz, 2H), 7.12 (t, J=8.7 Hz, 2H), 6.13 (s, 2H), 3.74 (s, 3H). m/z 237.1 [M+H]⁺.

### Step 4

A stirring suspension of methyl 5-amino-3-(4-fluorophenyl) isoxazole-4-carboxylate (5.00 g, 21.17 mmol) and cesium carbonate (13.79 g, 42.34 mmol) in DMF (100 mL) was cooled in an ice-bath and then treated with 1,3-dibromopropane (2.2 6 mL, 22.23 mmol) over 5 mins. The reaction was left to stir at rt for 5 h. Water (100 mL) was added to the reaction and then extracted with ether (3×100 mL). The combined organic extracts were washed with water (50 mL), brine (50 mL), dried over MgSO₄, filtered and concentrated under reduced pressure. The residue was adsorbed onto silica and purified by flash column chromatography (40 g silica, elution with a 0-100% EtOAc/PE gradient) to afford methyl 5-(azetidin-1-yl)-3-(4-fluorophenyl) isoxazole-4-carboxylate (2.02 g, 33% yield) as a yellow oil. ¹H NMR (500 MHZ, Chloroform-d) δ7.56 (dd, J=8.6, 5.6 Hz, 2H), 7.10 (t, J=8.8 Hz, 2H), 4.43 (t, J=7.8 Hz, 4H), 3.62 (s, 3H), 2.47 (pentet, J=7.8 Hz, 2H). m/z 277.0 [M+H]⁺.

### Step 5

To a solution of methyl 5-(azetidin-1-yl)-3-(4-fluorophenyl) isoxazole-4-carboxylate (2.02 g, 7.31 mmol) in THF (8 mL) and MeOH (8 mL) was added 2M NaOH (8.01 mL, 16.01 mmol) and the reaction mixture was stirred at rt 60° C. o/n. Upon completion, the volatiles were removed under reduced pressure and the aqueous solution was acidified with 2M HCl to pH 2-3. The solids were collected under vacuum filtration, washed with water (50 mL) and dried in a vacuum oven (40° C.) for 3 h to afford 5-(azetidin-1-yl)-3-(4-fluorophenyl) isoxazole-4-carboxylic acid (1.70 g, 84% yield) as a white solid. ¹H NMR (500 MHZ, DMSO-d₆) δ12.09 (s, 1H), 7.56 (dd, J=8.6, 5.7 Hz, 2H), 7.26 (t, J=8.9 Hz, 2H), 4.31 (t, J=7.7 Hz, 4H), 2.37 (pentet, J=7.7 Hz, 2H). m/z 263.0 [M+H]⁺.

### Step 6

To a stirring solution of 5-(azetidin-1-yl)-3-(4-fluorophenyl) isoxazole-4-carboxylic acid (671 mg, 2.56 mmol) in DMF (5 ml) was added oxalyl chloride (0.21 ml, 2.5 mmol). After 15 mins, the latter mixture was added to a solution of 6-bromo-2-methoxy-3-pyridinamine (400 mg, 1.97 mmol) and Et₃N (0.83 ml, 5.9 mmol) in DCM (3 mL) at 0° C. and the reaction mixture was left to stir at rt o/n. The reaction mixture was diluted with DCM (25 mL) and was washed with water (25 mL), 1 M NaOH (25 mL), brine (25 mL), dried over MgSO₄, filtered and concentrated under reduced pressure. Precipitation was induced by addition of MeOH and this was recrystallised from MeOH. The solids were collected by vacuum filtration to afford 5-(azetidin-1-yl)-N-(6-bromo-2-methoxy-3-pyridyl)-3-(4-fluorophenyl) isoxazole-4-carboxamide (373 mg, 40% yield) as a colourless solid. ¹H NMR (500 MHZ, Chloroform-d) δ8.47 (d, J=8.2 Hz, 1H), 7.60-7.51 (m, 2H), 7.27-7.19 (m, 3H), 6.98 (d, J=8.1 Hz, 1H), 4.46 (t, J=7.7 Hz, 4H), 3.66 (s, 3H), 2.49 (pentet, J=7.7 Hz, 2H). m/z 446.9/448.9 [M+H]⁺.

### Step 7

A 25 mL microwave vial was charged with a stirrer bar, 5-(azetidin-1-yl)-N-(6-bromo-2-methoxy-3-pyridyl)-3-(4-fluorophenyl) isoxazole-4-carboxamide (0.20 g, 0.45 mmol), KF (78 mg, 1.3 mmol) and 4-isoxazoleboronic acid pinacol ester (87 mg, 0.45 mmol), and the solids were dissolved in 1,4-dioxane (8 mL) and water (2 mL). The mixture was degassed for 10 mins by bubbling nitrogen into the solution. Pd-118 (29 mg, 0.040 mmol) was added into the mixture and the vial was capped, and the reaction mixture was stirred at rt o/n. The reaction mixture was concentrated in vacuo and the residue partitioned between DCM (10 mL) and water (10 mL). The layers were separated and the aqueous phase was further extracted with DCM (2×10 mL). The combined organic extracts were dried over MgSO₄ and concentrated under reduced pressure. The residue was adsorbed onto silica (12 g silica, 0-100% EtOAc/PE gradient) to afford 5-(azetidin-1-yl)-3-(4-fluorophenyl)-N-(6-isoxazol-4-yl-2-methoxy-3-pyridyl) isoxazole-4-carboxamide (78 mg, 38% yield) as a tan solid. ¹H NMR (500 MHz, Chloroform-d) δ8.78 (s, 1H), 8.64 (s, 1H), 8.60 (d, J=8.1 Hz, 1H), 7.58 (dd, J=8.4, 5.5 Hz, 2H), 7.40 (s, 1H), 7.26-7.21 (m, 2H), 7.01 (d, J=8.1 Hz, 1H), 4.48 (t, J=7.7 Hz, 4H), 3.70 (s, 3H), 2.50 (pentet, J=7.7 Hz, 2H). m/z 436.2 [M+H]⁺.

### Example 211 N-[6-[2-(Aminomethyl)pyrimidin-5-yl]-2-methoxy-3-pyridyl]-5-(3-chloropropylamino)-3-(4-fluorophenyl) isoxazole-4-carboxamide hydrochloride

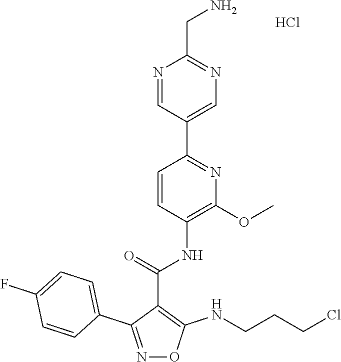 [SMILES: COc1nc(-c2cnc(CN)nc2)ccc1NC(=O)c1c(-c2ccc(F)cc2)noc1NCCCCl ; Cl]

### Step 1

In a microwave vial was added 5-(azetidin-1-yl)-N-(6-bromo-2-methoxy-3-pyridyl)-3-(4-fluorophenyl) isoxazole-4-carboxamide (79 mg, 0.18 mmol) (synthesised as described in Steps 1-6, Example 210), bis(pinacolato)diboron (58 mg, 0.23 mmol) and KOAc (52 mg, 0.53 mmol) in 1,4-dioxane (1.5 mL). The mixture was degassed by stirring under vacuum for 5 min, and then Pd-118 (15 mg, 0.020 mmol) was added and the reaction mixture was heated at 90° C. for 2 h. The reaction mixture was allowed to cool and the solvent was removed under reduced pressure. The crude residue was dissolved in EtOAc (10 mL) and washed with water (10 mL). The layers were separated and the organic layer was dried over MgSO₄, filtered and concentrated under reduced pressure to give a crude product which was triturated thrice with PE (2×10 mL). The residual solvent was removed under reduced pressure to afford 5-(azetidin-1-yl)-3-(4-fluorophenyl)-N-[2-methoxy-6-(4,4,5,5-tetramethyl-1,3,2-dioxaborolan-2-yl)-3-pyridyl] isoxazole-4-carboxamide (89 mg, 91% yield) as a brown solid. ¹H NMR (500 MHZ, Chloroform-d) δ8.51 (d, J=7.9 Hz, 1H), 7.56 (dd, J=8.5, 5.4 Hz, 2H), 7.50 (s, 1H), 7.40 (d, J=7.8 Hz, 1H), 7.20 (t, J=8.6 Hz, 2H), 4.46 (t, J=7.7 Hz, 4H), 3.72 (s, 3H), 2.48 (pentet, J=7.7 Hz, 2H), 1.34 (s, 12H). m/z 413.1 [M+H for boronic acid]⁺.

### Step 2

A 5 mL microwave vial was charged with a stirrer bar, Intermediate 11 (47 mg, 0.16 mmol), 5-(azetidin-1-yl)-3-(4-fluorophenyl)-N-[2-methoxy-6-(4,4,5,5-tetramethyl-1,3,2-dioxaborolan-2-yl)-3-pyridyl] isoxazole-4-carboxamide (89 mg, 0.16 mmol) and KF (28 mg, 0.48 mmol), and the solids were dissolved in 1,4-dioxane (1.5 mL) and water (0.15 mL). The mixture was degassed for 10 mins by bubbling nitrogen into the solution. Pd-118 (5 mg, 0.01 mmol) was added into the mixture and the vial was capped, and the reaction mixture was stirred at rt. Water (10 mL) was added and the mixture was extracted with DCM (3×10 mL). The combined organic extracts were dried over MgSO₄, filtered and concentrated under reduced pressure. The residue was adsorbed onto silica and purified by flash column chromatography (12 g silica, elution with a 0-100% EtOAc/PE gradient) to afford tert-butyl N-[[5-[5-[[5-(azetidin-1-yl)-3-(4-fluorophenyl) isoxazole-4-carbonyl]amino]-6-methoxy-2-pyridyl]pyrimidin-2-yl]methyl]carbamate (49 mg, 50% yield) as a white solid. ¹H NMR (500 MHz, Chloroform-d) δ9.19 (s, 2H), 8.69 (d, J=8.1 Hz, 1H), 7.59 (dd, J=8.6, 5.4 Hz, 2H), 7.45 (s, 1H), 7.30 (d, J=8.1 Hz, 1H), 7.28-7.23 (m, 2H), 5.70 (s, 1H), 4.63 (d, J=5.1 Hz, 2H), 4.49 (t, J=7.7 Hz, 4H), 3.75 (s, 3H), 2.50 (pentet, J=7.7 Hz, 2H), 1.48 (s, 9H). m/z 576.2 [M+H]⁺.

### Step 3

To a solution of tert-butyl N-[[5-[5-[[5-(azetidin-1-yl)-3-(4-fluorophenyl) isoxazole-4-carbonyl]amino]-6-methoxy-2-pyridyl]pyrimidin-2-yl]methyl]carbamate (49 mg, 0.090 mmol) in DCM (1 mL) was added a solution of HCl (4 N in 1,4-dioxane, 0.51 mL, 2.1 mmol) and the reaction mixture was left to stir at rt until reaction was complete by LCMS. An additional portion of HCl was added and the reaction mixture was left to stir for a total of 21 h. The volatiles were removed under reduced pressure, triturated in EtOAc and the solid was dried in a vacuum oven (40° C.) overnight to afford N-[6-[2-(aminomethyl)pyrimidin-5-yl]-2-methoxy-3-pyridyl]-5-(3-chloropropylamino)-3-(4-fluorophenyl) isoxazole-4-carboxamide hydrochloride (35 mg, 70% yield) as an orange solid. ¹H NMR (500 MHZ, DMSO-d₆) δ9.42 (s, 2H), 8.66 (d, J=8.2 Hz, 1H), 8.45 (s, 3H), 8.30 (t, J=6.4 Hz, 1H), 7.79 (d, J=8.2 Hz, 1H), 7.75 (dd, J=8.4, 5.5 Hz, 2H), 7.54 (t, J=8.8 Hz, 2H), 7.49 (s, 1H), 4.39-4.29 (m, 2H), 3.76-3.72 (m, 5H), 3.62-3.52 (m, 2H), 2.15-2.05 (m, 2H). m/z 512.1 [M+H for free base]⁺.

### Example 212 N-(6-Imidazol-1-yl-2-oxo-1H-pyridin-3-yl)-5-methyl-3-phenyl-isoxazole-4-carboxamide

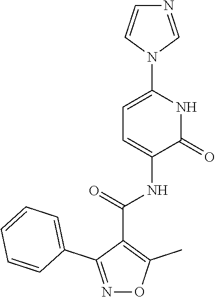 [SMILES: Cc1onc(-c2ccccc2)c1C(=O)Nc1ccc(-n2ccnc2)[nH]c1=O]

To solution of Example 1 (36 mg, 0.10 mmol) in DCM (2 mL) in an ice-bath was added 1 M BBr₃ in DCM (0.48 mL, 0.48 mmol) slowly while keeping the reaction temperature at 5° C. After 15 mins, MeOH (0.5 mL) was added to quench the excess BBr₃. The mixture was evaporated to dryness and the residue was purified by flash column chromatography (4 g silica, 1-2% MeOH/DCM gradient) to give N-(6-imidazol-1-yl-2-oxo-1H-pyridin-3-yl)-5-methyl-3-phenyl-isoxazole-4-carboxamide (23 mg, 63% yield) as a light brown solid. ¹H NMR (600 MHZ, DMSO-d₆) δ12.38 (s, 1H), 9.75 (s, 1H), 9.56 (s, 1H), 8.46 (d, J=8.0 Hz, 1H), 8.21 (s, 1H), 7.82 (s, 1H), 7.69 (d, J=6.9 Hz, 2H), 7.52-7.46 (m, 3H), 7.44 (d, J=8.3 Hz, 1H), 2.63 (s, 3H). m/z 362.1 [M+H]⁺.

### Example 213 N-[6-(2-Aminopyrimidin-5-yl)-2-methoxy-3-pyridyl]-3-(4-fluorophenyl)-5-methyl-isoxazole-4-carboxamide

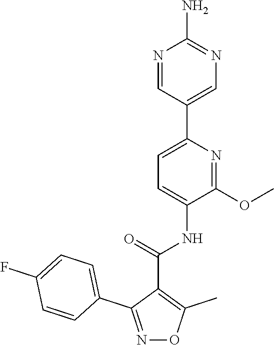 [SMILES: COc1nc(-c2cnc(N)nc2)ccc1NC(=O)c1c(-c2ccc(F)cc2)noc1C]

A mixture of 5-(4,4,5,5-tetramethyl-1,3,2-dioxaborolan-2-yl)-2-pyrimidinamine (109 mg, 0.49 mmol), Intermediate 12 (200 mg, 0.49 mmol) and potassium fluoride (87 mg, 1.48 mmol) in 1,4-dioxane (4 mL) and water (0.4 mL) was degassed by purging with N₂ gas for 5 min. Pd-118 (16 mg, 0.02 mmol) was added and the reaction mixture was stirred at room temperature for 3 h. Another portion of 5-(4,4,5,5-tetramethyl-1,3,2-dioxaborolan-2-yl)-2-pyrimidinamine and Pd-118 were added and the reaction mixture was stirred at 50° C. for 16 h. The reaction mixture was extracted with EtOAc (3×10 mL) and the combined organic extracts were concentrated under reduced pressure. Purification by flash silica column chromatography (elution with a 0-100% EtOAc/pet. ether gradient) yielded N-[6-(2-aminopyrimidin-5-yl)-2-methoxy-3-pyridyl]-3-(4-fluorophenyl)-5-methyl-isoxazole-4-carboxamide (85 mg, 39%) as an off-white solid. ¹H NMR (500 MHZ, DMSO-d₆) δ9.57 (s, 1H), 8.91 (s, 2H), 8.25 (d, J=8.0 Hz, 1H), 7.78 (t, J=7.1 Hz, 2H), 7.50 (d, J=8.1 Hz, 1H), 7.40 (t, J=8.8 Hz, 2H), 7.00 (s, 2H), 3.91 (s, 3H), 2.65 (s, 3H). m/z 421.1 [M+H]⁺.

### Example 214 N-[6-(2-Aminopyrimidin-5-yl)-2-isopropoxy-3-pyridyl]-5-methyl-3-phenyl-isoxazole-4-carboxamide

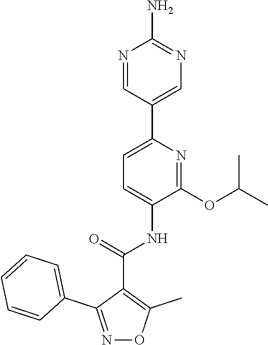 [SMILES: Cc1onc(-c2ccccc2)c1C(=O)Nc1ccc(-c2cnc(N)nc2)nc1OC(C)C]

### Step 1

A solution of N-(6-bromo-2-isopropoxy-3-pyridyl)-5-methyl-3-phenyl-isoxazole-4-carboxamide (602 mg, 1.45 mmol) (synthesised according to Step 1 in Example 206), B₂pin₂ (441 mg, 1.74 mmol) and KOAc (430 mg, 4.34 mmol) in 1,4-dioxane (11 mL) was degassed for 5 min by purging with N₂ gas. Pd(dppf)Cl₂ (106 mg, 0.14 mmol) was added and the reaction mixture was heated at 90° C. for 3 h. The solvent was removed under reduced pressure and the residue was partitioned between EtOAc (20 mL) and water (20 mL). The aqueous layer was extracted with EtOAc (2×20 mL). The combined organic extracts were dried over MgSO₄, filtered and concentrated under reduced pressure to yield a brown solid, which was triturated with petroleum ether to yield N-[2-isopropoxy-6-(4,4,5,5-tetramethyl-1,3,2-dioxaborolan-2-yl)-3-pyridyl]-5-methyl-3-phenyl-isoxazole-4-carboxamide (669 mg, 95%) as a brown solid, which was used in the next step without further purification. ¹H NMR (500 MHZ, CDCl₃) δ8.63 (d, J=7.8 Hz, 1H), 7.84 (s, 1H), 7.68-7.62 (m, 2H), 7.56-7.46 (m, 3H), 7.43 (dd, J=7.8, 0.5 Hz, 1H), 5.38 (hept, J=6.2 Hz, 1H), 2.77 (s, 3H), 1.33 (s, 12H), 0.96 (d, J=6.2 Hz, 6H). m/z 382.1 [M+H, boronic acid]⁺.

### Step 2

A mixture of N-[2-isopropoxy-6-(4,4,5,5-tetramethyl-1,3,2-dioxaborolan-2-yl)-3-pyridyl]-5-methyl-3-phenyl-isoxazole-4-carboxamide (300 mg, 0.62 mmol), 2-amino-5-bromopyrimidine (112 mg, 0.65 mmol) and KF (109 mg, 1.85 mmol) in 1,4-dioxane (5 mL) and water (0.5 mL) was degassed for 5 min. Pd-118 (20 mg, 0.03 mmol) was added and the reaction mixture was heated at 80° C. for 1 h. The volatiles were removed under reduced pressure and the residue was partitioned between EtOAc (20 mL) and water (20 mL), and separated. The aqueous layer was extracted with EtOAc (2×20 mL) and the combined organic extracts were dried over MgSO₄, filtered and concentrated under reduced pressure. Purification by flash silica column chromatography (elution with a 0-5% MeOH/CH₂Cl₂ gradient) yielded a brown solid, which was then further purified by flash silica column chromatography (elution with a 0-100% EtOAc/CH₂Cl₂ gradient) to yield N-[6-(2-aminopyrimidin-5-yl)-2-isopropoxy-3-pyridyl]-5-methyl-3-phenyl-isoxazole-4-carboxamide (89 mg, 32%) as an off-white solid. ¹H NMR (500 MHZ, DMSO-d₆) δ9.39 (s, 1H), 8.86 (s, 2H), 8.23 (d, J=8.1 Hz, 1H), 7.73 (d, J=7.0 Hz, 2H), 7.59-7.48 (m, 3H), 7.45 (d, J=8.1 Hz, 1H), 6.98 (s, 2H), 5.34 (hept, J=6.0 Hz, 1H), 2.68 (s, 3H), 1.26 (d, J=6.1 Hz, 6H). m/z 431.2 [M+H]⁺.

### Example 215 N-[6-(6-Amino-3-pyridyl)-2-isopropoxy-3-pyridyl]-5-methyl-3-phenyl-isoxazole-4-carboxamide

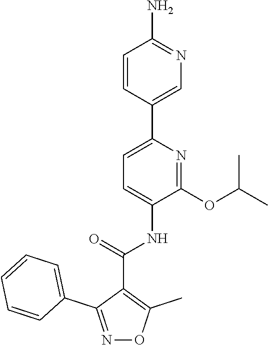 [SMILES: Cc1onc(-c2ccccc2)c1C(=O)Nc1ccc(-c2ccc(N)nc2)nc1OC(C)C]

### Step 1

A mixture of N-[2-isopropoxy-6-(4,4,5,5-tetramethyl-1,3,2-dioxaborolan-2-yl)-3-pyridyl]-5-methyl-3-phenyl-isoxazole-4-carboxamide (351 mg, 0.72 mmol) (synthesised according to Step 1 in Example 214), tert-butyl (5-bromopyridin-2-yl) carbamate (196 mg, 0.72 mmol) and KF (128 mg, 2.16 mmol) in 1,4-dioxane (6 mL) and water (0.6 mL) was degassed for 5 min. Pd-118 (24 mg, 0.04 mmol) was added and the reaction mixture was heated at 90° C. for 2 h. The solvent was removed under reduced pressure and the material was purified by flash silica column chromatography (elution with a 0-100% EtOAc/CH₂Cl₂ gradient), followed by trituration in EtOAc to yield tert-butyl N-[5-[6-isopropoxy-5-[(5-methyl-3-phenyl-isoxazole-4-carbonyl)amino]-2-pyridyl]-2-pyridyl]carbamate (240 mg, 60%) as a cream solid. ¹H NMR (500 MHZ, DMSO-d₆) δ9.98 (s, 1H), 9.40 (s, 1H), 8.90 (d, J=2.7 Hz, 1H), 8.36-8.27 (m, 2H), 7.88 (d, J=8.9 Hz, 1H), 7.77-7.68 (m, 2H), 7.60-7.47 (m, 4H), 5.37 (hept, J=6.1 Hz, 1H), 2.68 (s, 3H), 1.49 (s, 9H), 1.27 (d, J=6.2 Hz, 6H). m/z 530.2 [M+H]⁺.

### Step 2

A solution of tert-butyl N-[5-[6-isopropoxy-5-[(5-methyl-3-phenyl-isoxazole-4-carbonyl)amino]-2-pyridyl]-2-pyridyl]carbamate (240 mg, 0.43 mmol) in CH₂Cl₂/TFA (5 mL, 1:1) was stirred at room temperature for 3 h. The solution was diluted with CH₂Cl₂ (10 mL) and water (10 mL), then the aqueous layer was basified with 2 M NaOH. The layers were separated and the aqueous layer was extracted with CH₂Cl₂ (2×20 mL). The combined organic extracts were washed with brine (20 mL), dried over MgSO₄, filtered and concentrated under reduced pressure to yield N-[6-(6-amino-3-pyridyl)-2-isopropoxy-3-pyridyl]-5-methyl-3-phenyl-isoxazole-4-carboxamide (194 mg, 100%) as a cream solid. ¹H NMR (500 MHZ, DMSO-d₆) δ9.35 (s, 1H), 8.61 (d, J=2.6 Hz, 1H), 8.19 (d, J=8.1 Hz, 1H), 8.00 (dd, J=8.7, 2.5 Hz, 1H), 7.78-7.68 (m, 2H), 7.59-7.48 (m, 3H), 7.38 (d, J=8.1 Hz, 1H), 6.50 (d, J=8.7 Hz, 1H), 6.26 (s, 2H), 5.34 (hept, J=5.9 Hz, 1H), 2.68 (s, 3H), 1.26 (d, J=6.2 Hz, 6H). m/z 430.2 [M+H]⁺.

### Example 216 N-[6-(2-Aminopyrimidin-5-yl)-2-(oxetan-3-yloxy)-3-pyridyl]-3-(4-fluorophenyl)-5-methyl-isoxazole-4-carboxamide

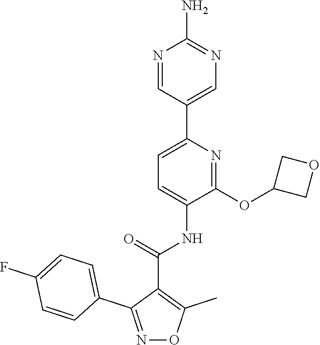 [SMILES: Cc1onc(-c2ccc(F)cc2)c1C(=O)Nc1ccc(-c2cnc(N)nc2)nc1OC1COC1]

### Step 1

To an ice-cooled solution of oxetan-3-ol (89 mg, 1.2 mmol) in 1,4-dioxane (4 mL) was added NaH (56 mg, 1.4 mmol, 60% in mineral oil) over 5 portions. The mixture was stirred at room temperature for 15 min, then 2,6-dibromopyridin-3-amine (252 mg, 1.0 mmol) was added in one portion and the reaction mixture was heated at 100° C. After 17 h, the reaction mixture was treated with oxetan-3-ol (222 mg, 3.0 mmol) and NaH (120 mg, 3.0 mmol, 60% in mineral oil), and the reaction mixture was stirred at 100° C. for 21 h. The solvent was removed under reduced pressure and the residue was partitioned between CH₂Cl₂ (20 mL) and saturated aqueous NH₄Cl (20 mL), then passed through a hydrophobic slit phase separator. The aqueous layer was washed with CH₂Cl₂ (20 mL) and the combined filtrate was concentrated under reduced pressure to yield a brown oil. Purification by flash silica column chromatography (elution with a 0-100% EtOAc/petroleum ether gradient) yielded 6-bromo-2-(oxetan-3-yloxy)pyridin-3-amine (77 mg, 31%) as an orange oil. ¹H NMR (500 MHZ, CDCl₃) δ6.89 (d, J=7.9 Hz, 1H), 6.80 (d, J=7.9 Hz, 1H), 5.64 (tt, J=6.3, 5.3 Hz, 1H), 5.03-4.98 (m, 2H), 4.76-4.72 (m, 2H), 3.80 (s, 2H). m/z 244.9, 246.9 [M+H]⁺.

### Step 2

To a solution of 6-bromo-2-(oxetan-3-yloxy)pyridin-3-amine (78 mg, 0.31 mmol) and DIPEA (0.080 mL, 0.47 mmol) in CH₂Cl₂ (5 mL) was added a 0.4 M solution of 3-(4-fluorophenyl)-5-methyl-isoxazole-4-carbonyl chloride (0.78 mL, 0.31 mmol) in CH₂Cl₂ (synthesised using appropriate starting materials by the method described in Step 1 of Example 1). The reaction mixture was stirred at room temperature for 21 h and then diluted with CH₂Cl₂ (10 mL) and water (20 mL). The mixture was stirred vigorously for 5 min, then passed through a hydrophobic slit phase separator. The aqueous phase was washed with CH₂Cl₂ (20 mL) and the filtrate was concentrated under reduced pressure. Purification by flash silica column chromatography (elution with a 0-100% EtOAc/CH₂Cl₂ gradient) yielded N-[6-bromo-2-(oxetan-3-yloxy)-3-pyridyl]-3-(4-fluorophenyl)-5-methyl-isoxazole-4-carboxamide (67 mg, 45%) as a colourless solid. ¹H NMR (500 MHZ, CDCl₃) δ8.63 (d, J=8.3 Hz, 1H), 7.70-7.63 (m, 2H), 7.54 (s, 1H), 7.26-7.20 (m, 2H), 7.10 (dd, J=8.3, 0.5 Hz, 1H), 5.43 (tt, J=6.3, 5.3 Hz, 1H), 4.87-4.80 (m, 2H), 4.31-4.24 (m, 2H), 2.79 (s, 3H). m/z 448.0, 450.0 [M+H]⁺.

### Step 3

A mixture of N-[6-bromo-2-(oxetan-3-yloxy)-3-pyridyl]-3-(4-fluorophenyl)-5-methyl-isoxazole-4-carboxamide (67 mg, 0.14 mmol), 5-(4,4,5,5-tetramethyl-1,3,2-dioxaborolan-2-yl)-2-pyrimidinamine (33 mg, 0.15 mmol) and KF (25 mg, 0.43 mmol) in 1,4-dioxane (1.5 mL) and water (0.15 mL) was degassed by purging with N₂ gas for 5 min. Pd-118 (4.6 mg, 0.007 mmol) was added and the reaction mixture was heated at 90° C. for 4 h. Additional 5-(4,4,5,5-tetramethyl-1,3,2-dioxaborolan-2-yl)-2-pyrimidinamine (15.7 mg, 0.07 mmol) was added and the reaction was stirred at 90° C. for 16 h. The volatiles were removed under reduced pressure and purification by flash silica column chromatography (elution with a 0-100% EtOAc/CH₂Cl₂ gradient) yielded an off-white solid, which was then purified by flash silica column chromatography (elution with a 0-5% MeOH/EtOAc gradient) to yield N-[6-(2-aminopyrimidin-5-yl)-2-(oxetan-3-yloxy)-3-pyridyl]-3-(4-fluorophenyl)-5-methyl-isoxazole-4-carboxamide (12 mg, 17%) as an off-white solid. ¹H NMR (500 MHZ, DMSO-d₆) δ9.74 (s, 1H), 8.83 (s, 2H), 8.30 (d, J=8.1 Hz, 1H), 7.80 (t, J=7.1 Hz, 2H), 7.53 (d, J=8.1 Hz, 1H), 7.37 (t, J=8.9 Hz, 2H), 6.98 (s, 2H), 5.68 (p, J=5.7 Hz, 1H), 4.91 (t, J=6.8 Hz, 2H), 4.53 (dd, J=7.2, 5.6 Hz, 2H), 2.68 (s, 3H). m/z 463.2 [M+H]⁺.

### Example 217 N-[6-(2-Aminopyrimidin-5-yl)-2-(2-methoxyethoxy)-3-pyridyl]-3-(4-fluorophenyl)-5-methyl-isoxazole-4-carboxamide

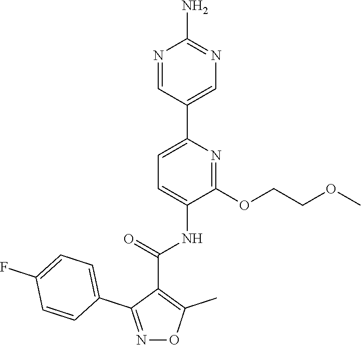 [SMILES: COCCOc1nc(-c2cnc(N)nc2)ccc1NC(=O)c1c(-c2ccc(F)cc2)noc1C]

### Step 1

NaH (640 mg, 16.0 mmol, 60% in mineral oil) was charged in a 25 mL microwave vial and was washed with petroleum ether to remove the mineral oil. The solids were suspended in THF (3 mL) and then 2-methoxyethanol (3 mL) was added at 0° C. After stirring at room temperature for 2 h, 2,6-dibromopyridin-3-amine (504 mg, 2.0 mmol) was added to the light brown solution and the reaction mixture was heated at 80° C. Saturated aqueous NH₄Cl (20 mL) was added and the organic solvent was removed under reduced pressure. CH₂Cl₂ (20 mL) was added to the aqueous mixture and then passed through a hydrophobic slit phase separator. The aqueous layer was extracted with CH₂Cl₂ (20 mL) and the combined organic extracts were concentrated under reduced pressure. Purification by flash silica column chromatography (elution with a 0-100% EtOAc/petroleum ether gradient) yielded 6-bromo-2-(2-methoxyethoxy)pyridin-3-amine (431 mg, 87%) as an orange solid. ¹H NMR (500 MHZ, CDCl₃) δ6.90 (s, 2H), 4.54-4.49 (m, 2H), 3.78-3.74 (m, 2H), 3.42 (s, 3H). m/z 247.0, 249.0 [M+H]⁺.

### Step 2

To an ice-cooled solution of 6-bromo-2-(2-methoxyethoxy)pyridin-3-amine (431 mg, 1.74 mmol) and DIPEA (0.45 mL, 2.62 mmol) in CH₂Cl₂ (4 mL) was added a solution of 3-(4-fluorophenyl)-5-methyl-isoxazole-4-carbonyl chloride (1.74 mmol) in CH₂Cl₂ (4 mL) (synthesised using appropriate starting materials by the method described in Step 1 of Example 1). The reaction was stirred for 18 h and then water (10 mL) was added to the reaction and stirred for 5 min, then separated. The aqueous layer was extracted with CH₂Cl₂ (2×10 mL) and the combined organic extracts were dried over MgSO₄, filtered and concentrated under reduced pressure. Purification by flash silica column chromatography (elution with a 30-100% EtOAc/petroleum ether gradient) yielded N-[6-bromo-2-(2-methoxyethoxy)-3-pyridyl]-3-(4-fluorophenyl)-5-methyl-isoxazole-4-carboxamide (660 mg, 84%) as an off-white solid. ¹H NMR (500 MHZ, DMSO-d₆) δ9.73 (s, 1H), 8.12 (d, J=8.1 Hz, 1H), 7.78 (dd, J=8.6, 5.4 Hz, 2H), 7.42-7.31 (m, 2H), 7.26 (d, J=8.1 Hz, 1H), 4.44-4.31 (m, 2H), 3.66-3.57 (m, 2H), 3.26 (s, 3H), 2.65 (s, 3H). m/z 450.0, 452.0 [M+H]⁺.

### Step 3

A mixture of N-[6-bromo-2-(2-methoxyethoxy)-3-pyridyl]-3-(4-fluorophenyl)-5-methyl-isoxazole-4-carboxamide (360 mg, 0.80 mmol), 5-(4,4,5,5-tetramethyl-1,3,2-dioxaborolan-2-yl)-2-pyrimidinamine (186 mg, 0.84 mmol) and KF (142 mg, 2.40 mmol) in 1,4-dioxane (7 mL) and water (0.7 mL) was degassed for 5 min. Pd-118 (26 mg, 0.040 mmol) was added and the reaction mixture was heated at 80° C. for 16 h. Pd-118 (26 mg, 0.040 mmol) and 5-(4,4,5,5-tetramethyl-1,3,2-dioxaborolan-2-yl)-2-pyrimidinamine (88 mg, 0.40 mmol) were added and the reaction mixture was heated at 80° C. for 1 h. The volatiles were removed under reduced pressure and purification by flash silica column chromatography (elution with a 0-100% EtOAc/CH₂Cl₂ gradient) yielded N-[6-(2-aminopyrimidin-5-yl)-2-(2-methoxyethoxy)-3-pyridyl]-3-(4-fluorophenyl)-5-methyl-isoxazole-4-carboxamide (224 mg, 57%) as an off-white solid. ¹H NMR (500 MHZ, DMSO-d₆) δ9.63 (s, 1H), 8.89 (s, 2H), 8.19 (d, J=8.0 Hz, 1H), 7.80 (t, J=7.1 Hz, 2H), 7.49 (d, J=8.0 Hz, 1H), 7.43-7.32 (m, 2H), 6.97 (s, 2H), 4.51 (t, J=4.9 Hz, 2H), 3.70-3.61 (m, 2H), 3.27 (s, 3H), 2.67 (s, 3H). m/z 465.1 [M+H]⁺.

### Example 218 N-[6-(2-Aminopyrimidin-5-yl)-2-(2-hydroxyethoxy)-3-pyridyl]-3-(4-fluorophenyl)-5-methyl-isoxazole-4-carboxamide

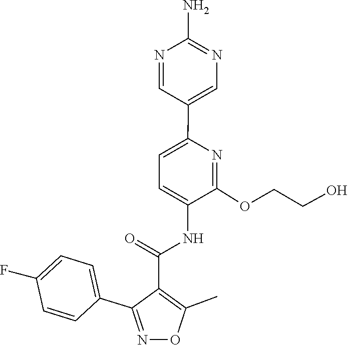 [SMILES: Cc1onc(-c2ccc(F)cc2)c1C(=O)Nc1ccc(-c2cnc(N)nc2)nc1OCCO]

### Step 1

To a solution of N-[6-bromo-2-(2-methoxyethoxy)-3-pyridyl]-3-(4-fluorophenyl)-5-methyl-isoxazole-4-carboxamide (300 mg, 0.67 mmol) (synthesised according to Step 1 and 2 in Example 217) in CH₂Cl₂ (6 mL) was added at −78° C. a 1 M solution of boron tribromide in CH₂Cl₂ (0.70 mL, 0.70 mmol) over 2 min and the reaction mixture was warmed to room temperature and stirred for 19 h. Saturated aqueous NaHCO₃ (20 mL) was added and the mixture was extracted with CH₂Cl₂ (3×10 mL). The combined organic extracts were washed with brine (10 mL), dried over MgSO₄, filtered and concentrated under reduced pressure. Purification by flash silica column chromatography (elution with a 30-100% EtOAc/petroleum ether gradient) yielded N-[6-bromo-2-(2-hydroxyethoxy)-3-pyridyl]-3-(4-fluorophenyl)-5-methyl-isoxazole-4-carboxamide (166 mg, 51%) as a colourless solid. ¹H NMR (400 MHZ, DMSO-d₆) δ9.68 (s, 1H), 8.26 (d, J=8.1 Hz, 1H), 7.76 (dd, J=8.2, 5.7 Hz, 2H), 7.42-7.30 (m, 2H), 7.25 (d, J=8.1 Hz, 1H), 4.76 (t, J=6.0 Hz, 1H), 4.24 (t, J=4.9 Hz, 2H), 3.65 (q, J=5.4 Hz, 2H), 2.65 (s, 3H). m/z 458.0, 459.9 [M+Na]⁺.

### Step 2

A mixture of N-[6-bromo-2-(2-hydroxyethoxy)-3-pyridyl]-3-(4-fluorophenyl)-5-methyl-isoxazole-4-carboxamide (166 mg, 0.34 mmol), 5-(4,4,5,5-tetramethyl-1,3,2-dioxaborolan-2-yl)-2-pyrimidinamine (79 mg, 0.36 mmol) and KF (61 mg, 1.03 mmol) in 1,4-dioxane (3 mL) and water (0.3 mL) was degassed for 5 min. Pd-118 (11 mg, 0.017 mmol) was added and the reaction mixture was heated at 80° C. for 16 h. Pd-118 (11 mg, 0.017 mmol) and 5-(4,4,5,5-tetramethyl-1,3,2-dioxaborolan-2-yl)-2-pyrimidinamine (38 mg, 0.17 mmol) were added and the reaction mixture was heated at 80° C. for 1 h. The volatiles were removed under reduced pressure and purification by flash silica column chromatography (elution with a 0-100% EtOAc/CH₂Cl₂ gradient) yielded an off-white. The material was triturated in water (50 mL) and the solids were collected by vacuum filtration, washed with IPA and dried in vacuum oven to yield N-[6-(2-aminopyrimidin-5-yl)-2-(2-hydroxyethoxy)-3-pyridyl]-3-(4-fluorophenyl)-5-methyl-isoxazole-4-carboxamide (77 mg, 47%) as a colourless solid. ¹H NMR (500 MHZ, DMSO-d₆) δ9.62 (s, 1H), 8.88 (s, 2H), 8.33 (d, J=8.1 Hz, 1H), 7.78 (t, J=7.1 Hz, 2H), 7.48 (d, J=8.1 Hz, 1H), 7.37 (t, J=8.8 Hz, 2H), 6.96 (s, 2H), 4.77 (t, J=6.1 Hz, 1H), 4.37 (t, J=5.0 Hz, 2H), 3.69 (q, J=5.5 Hz, 2H), 2.67 (s, 3H). m/z 451.1 [M+H]⁺.

### Example 219 N-[6-[4-(hydroxymethyl)phenyl]-2-methoxy-3-pyridyl]-5-methyl-3-phenyl-isoxazole-4-carboxamide

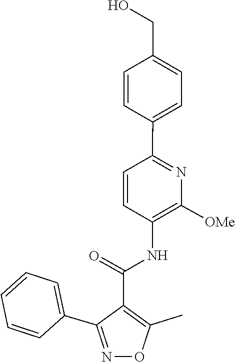 [SMILES: COc1nc(-c2ccc(CO)cc2)ccc1NC(=O)c1c(-c2ccccc2)noc1C]

Synthesised according to method described in Step 2 of Example 27, substituting 4-bromobenzyl alcohol for (5-bromopyrid-2-yl) methanol and heated at 45° C. for 18 hours. Afforded N-[6-[4-(hydroxymethyl)phenyl]-2-methoxy-3-pyridyl]-5-methyl-3-phenyl-isoxazole-4-carboxamide (370.0 mg, 75% yield) as a light brown, candy-floss like solid. ¹H NMR (500 MHZ, Chloroform-d) δ8.70 (d, J=8.2 Hz, 1H), 7.96 (d, J=8.0 Hz, 2H), 7.76 (s, 1H), 7.68-7.61 (m, 3H), 7.61-7.55 (m, 2H), 7.42 (d, J=8.2 Hz, 2H), 7.35 (d, J=8.2 Hz, 1H), 4.74 (d, J=5.4 Hz, 2H), 3.72 (d, J=0.9 Hz, 3H), 2.84 (d, J=0.9 Hz, 3H), 1.74 (t, J=5.8 Hz, 1H). m/z 416.2 [M+H]⁺.

### Example 220 N-[6-(4-formylphenyl)-2-methoxy-3-pyridyl]-5-methyl-3-phenyl-isoxazole-4-carboxamide

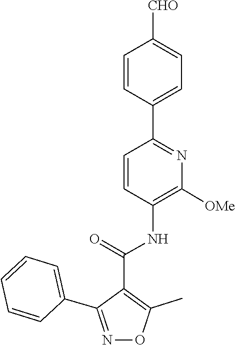 [SMILES: COc1nc(-c2ccc(C=O)cc2)ccc1NC(=O)c1c(-c2ccccc2)noc1C]

A suspension of Example 219 (360.0 mg, 0.9 mmol) and manganese (IV) oxide (850.0 mg, 9.8 mmol) in DCM (20 mL) was heated in a sealed tube to 60° C. for 2 hours. Filtered through Celite and the cake was washed with 80 mL DCM. Concentrated to dryness to give N-[6-(4-formylphenyl)-2-methoxy-3-pyridyl]-5-methyl-3-phenyl-isoxazole-4-carboxamide (330.0 mg, 88% yield) as a light brown solid. ¹H NMR (500 MHZ, Chloroform-d) δ10.05 (s, 1H), 8.77 (d, J=8.2 Hz, 1H), 8.14 (d, J=8.1 Hz, 2H), 7.93 (d, J=8.3 Hz, 2H), 7.82 (s, 1H), 7.69-7.62 (m, 3H), 7.62-7.56 (m, 2H), 7.46 (d, J=8.2 Hz, 1H), 3.74 (s, 3H), 2.86 (s, 3H). m/z 414.2 [M+H]⁺.

### Example 221 3-(4-fluorophenyl)-N-(6-(isoxazol-4-yl)-2-methoxypyridin-3-yl)-5-methylisoxazole-4-carboxamide

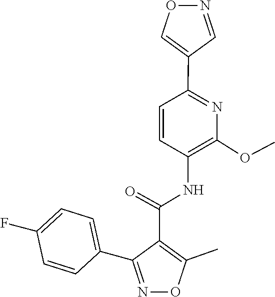 [SMILES: COc1nc(-c2cnoc2)ccc1NC(=O)c1c(-c2ccc(F)cc2)noc1C]

To a reaction vial was added 4-isoxazoleboronic acid pinacol ester (96.0 mg, 0.49 mmol), potassium fluoride (87.0 mg, 1.47 mmol), Intermediate 12 (200.0 mg, 0.49 mmol), 1,4-Dioxane (4.8 mL) and Water (4.8 mL). The mixture was degassed with nitrogen prior to the addition of bis[2-(di-tert-butylphosphanyl)cyclopenta-2,4-dien-1-yl] iron; dichloropalladium (32.0 mg, 0.05 mmol) and mixture stirred at room temperature overnight. An additional portion of 4-isoxazoleboronic acid pinacol ester (96.0 mg, 0.49 mmol) and bis[2-(di-tert-butylphosphanyl)cyclopenta-2,4-dien-1-yl] iron; dichloropalladium (32.0 mg, 0.05 mmol) were added, reaction degassed and stirred at rt for 72 h. The reaction was diluted with EtOAc, washed sequentially with water and brine, dried over sodium sulfate, filtered, and concentrated under reduced pressure. The crude material was purified by flash silica column chromatography on an ISCO system (0-90% petroleum ether/EtOAc gradient) to give 3-(4-fluorophenyl)-N-(6-(isoxazol-4-yl)-2-methoxypyridin-3-yl)-5-methylisoxazole-4-carboxamide (66 mg, 34% yield). ¹H NMR (500 MHZ, Chloroform-d) δ8.81 (s, 1H), 8.69 (d, J=8.1 Hz, 1H), 8.67 (s, 1H), 7.67 (s, 1H), 7.67-7.60 (m, 2H), 7.32-7.25 (m, 2H), 7.09-7.02 (m, 1H), 3.73 (s, 3H), 2.82 (s, 3H). m/z 393.2 [M−H].

### Example 222 N-(6′-amino-6-methoxy-[2,3′-bipyridin]-5-yl)-3-(4-fluorophenyl)-5-methylisoxazole-4-carboxamide

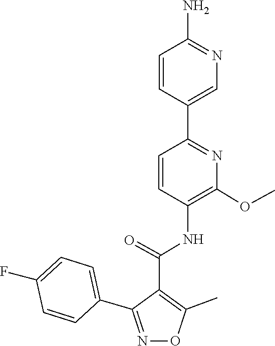 [SMILES: COc1nc(-c2ccc(N)nc2)ccc1NC(=O)c1c(-c2ccc(F)cc2)noc1C]

### Step 1

A solution of 2-(Boc-amino)-5-bromopyridine (200.0 mg, 0.73 mmol), bis(picacolato)diboron (241.7 mg, 0.95 mmol) and potassium acetate (217.8 mg, 2.20 mmol) in 1,4-Dioxane (2 mL) was degassed for 5 min by purging with N2. [1,1′-bis(diphenylphosphino) ferrocene] dichloropalladium (II) (26.9 mg, 0.04 mmol) was added and the reaction mixture was sealed and heated at 100° C. for 2 h. The solvent was removed under reduced pressure and resulting residue purified by flash silica column chromatography on an ISCO system (0-100% EtOAc/petroleum ether gradient) followed by trituration with petroleum ether to yield tert-butyl (5-(4,4,5,5-tetramethyl-1,3,2-dioxaborolan-2-yl)pyridin-2-yl) carbamate (140 mg, 60% yield) as a colourless solid. ¹H NMR (500 MHZ, CDCl₃) δ9.02 (s, 1H), 8.65 (dd, J=1.9, 1.0 Hz, 1H), 8.06 (dd, J=8.5, 1.8 Hz, 1H), 8.02 (dd, J=8.5, 1.1 Hz, 1H), 1.55 (s, 9H), 1.33 (s, 12H). m/z 183.0 [boronic acid+H-tert-butyl]⁺.

### Step 2

A mixture of tert-butyl N-[5-(4,4,5,5-tetramethyl-1,3,2-dioxaborolan-2-yl)-2-pyridyl]carbamate (137.0 mg, 0.43 mmol), Intermediate 12 (173.8 mg, 0.43 mmol) and potassium acetate (75.9 mg, 1.28 mmol) in Water (0.4 mL) and 1,4-Dioxane (4 mL) was degassed with N₂ bis[2-(di-tert-butylphosphanyl)cyclopenta-2,4-dien-1-yl] iron; dichloropalladium (14.0 mg, 0.02 mmol) was added and the reaction mixture heated at 80° C. for 18 h. The solvent was removed under reduced pressure and crude product purified by flash silica column chromatography on ISCO system (0-50% EtOAc/CH₂Cl₂ gradient) followed by trituration in EtOAc yielding tert-butyl (5-(3-(4-fluorophenyl)-5-methylisoxazole-4-carboxamido)-6-methoxy-[2,3′-bipyridin]-6′-yl) carbamate as an off-white solid. (137 mg, 55%). ¹H NMR (500 MHZ, DMSO) δ10.00 (s, 1H), 9.57 (s, 1H), 8.94 (d, J=2.7 Hz, 1H), 8.37 (dd, J=8.8, 2.5 Hz, 1H), 8.33 (d, J=8.1 Hz, 1H), 7.89 (d, J=8.8 Hz, 1H), 7.78 (t, J=7.0 Hz, 2H), 7.61 (d, J=8.1 Hz, 1H), 7.45-7.35 (m, 2H), 3.93 (s, 3H), 2.66 (s, 3H), 1.49 (s, 9H). m/z 520.2 [M+H]⁺.

### Step 3

A solution of tert-butyl N-[5-[5-[[3-(4-fluorophenyl)-5-methyl-isoxazole-4-carbonyl]amino]-6-methoxy-2-pyridyl]-2-pyridyl]carbamate (137.0 mg, 0.24 mmol) in DCM (2 mL) and trifluoroacetic acid (2 mL, 26.1 mmol) was stirred at room temperature for 16 h. The solution was diluted with DCM (10 mL) and water (10 mL), then 2 M NaOH was added until the aqueous layer was basic. The layers were separated and the aqueous layer was extracted with DCM (2×20 mL). The combined organic extracts were washed with brine (20 mL), dried over MgSO₄, filtered and concentrated under reduced pressure to yield N-(6′-amino-6-methoxy-[2,3′-bipyridin]-5-yl)-3-(4-fluorophenyl)-5-methylisoxazole-4-carboxamide (100 mg, 95% yield) as a yellow solid. ¹H NMR (500 MHZ, DMSO) δ9.51 (s, 1H), 8.64 (d, J=2.4 Hz, 1H), 8.20 (d, J=8.1 Hz, 1H), 8.04 (dd, J=8.6, 2.5 Hz, 1H), 7.84-7.71 (m, 2H), 7.48-7.33 (m, 3H), 6.51 (dd, J=8.7, 0.8 Hz, 1H), 6.27 (s, 2H), 3.90 (s, 3H), 2.65 (s, 3H). m/z 420.1 [M+H]⁺.

### Example 223 N-(2′-amino-4-methoxy-[2,5′-bipyrimidin]-5-yl)-5-methyl-3-phenylisoxazole-4-carboxamide

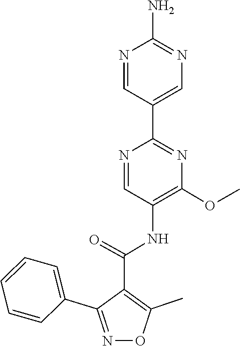 [SMILES: COc1nc(-c2cnc(N)nc2)ncc1NC(=O)c1c(-c2ccccc2)noc1C]

### Step 1

A 25% solution of sodium methoxide in methanol (2.1 mL, 9.1 mmol) was added dropwise to a solution of 2,4-dichloropyrimidin-5-ylamine (1.00 g, 6.1 mmol) in Methanol (12.5 mL) and the mixture was stirred at rt for 2 h. The mixture was diluted EtOAc and quenched with water. The organics were separated and aqueous extracted with EtOAc. Combined organics were washed with water, dried (MgSO₄), reduced in vacuo to yield an orange solid of 2-chloro-4-methoxypyrimidin-5-amine (0.9 g, 86% yield). ¹H NMR (500 MHZ, Chloroform-d) δ7.79 (s, 1H), 4.01 (s, 3H), 3.83 (d, J=29.6 Hz, 2H). m/z 160.0 [M+H]⁺.

### Step 2

To a stirring suspension of 2-chloro-4-methoxy-pyrimidin-5-amine (628.3 mg, 3.94 mmol) and 5-methyl-3-phenyl-1,2-oxazole-4-carboxylic acid (800.0 mg, 3.94 mmol) in DCM (16 mL) was added HATU (1.80 g, 4.72 mmol) followed by N-ethyldiisopropylamine (2.02 mL, 11.81 mmol) and reaction aged at rt overnight. The solvent was removed under reduced pressure and DCM added. NaHCO₃ was added and reaction stirred vigorously before being passed through a hydrophobic frit. The solvent was reduced under reduced pressure to yield a solid. The resulting solid was purified via column chromatography; (0-100% PE/EtOAc) to yield a white solid of N-(2-chloro-4-methoxypyrimidin-5-yl)-5-methyl-3-phenylisoxazole-4-carboxamide (268 mg, 19% yield). ¹H NMR (500 MHZ, Chloroform-d) δ9.42 (s, 1H), 7.72-7.64 (m, 1H), 7.64-7.58 (m, 4H), 7.46 (s, 1H), 3.73 (s, 3H), 2.86 (s, 3H). m/z 345.0 [M+H]⁺.

### Step 3

To a 5 mL microwave vial was added 5-(4,4,5,5-tetramethyl-1,3,2-dioxaborolan-2-yl)-2-pyrimidinamine (117.8 mg, 0.53 mmol), N-(2-chloro-4-methoxy-pyrimidin-5-yl)-5-methyl-3-phenyl-isoxazole-4-carboxamide (175.0 mg, 0.51 mmol) followed by Water (1 mL) and 1,4-Dioxane (4 mL) and the reaction mixture degassed with bubbling N2 before potassium fluoride (90.0 mg, 1.52 mmol) and bis[2-(di-tert-butylphosphanyl)cyclopenta-2,4-dien-1-yl]iron; dichloropalladium (16.6 mg, 0.03 mmol) was added. The reaction mixture was degassed for a further 10 minutes before being left stirring at 90° C. overnight. The reaction mixture was allowed to cool to rt before water and DCM were added. The organics were separated and aqueous extracted with DCM (×3). The organics were combined, passed through a hydrophobic frit and reduced in vacuo. The resulting solid was dissolved in DCM and loaded onto celite. The crude mixture was purified via column chromatography (0-10% DCM-MeOH gradient) to yield N-(2′-amino-4-methoxy-[2,5′-bipyrimidin]-5-yl)-5-methyl-3-phenylisoxazole-4-carboxamide as a beige solid (131 mg, 61%). ¹H NMR (500 MHZ, Chloroform-d) δ9.48 (s, 1H), 9.18 (s, 2H), 7.63-7.57 (m, 1H), 7.57-7.51 (m, 4H), 7.48 (s, 1H), 6.31 (s, 2H), 3.68 (s, 3H), 2.79 (s, 3H). m/z 404.1 [M+H]⁺.

### Example 224 N-[6-[4-(chloromethyl)phenyl]-2-methoxy-3-pyridyl]-5-methyl-3-phenyl-isoxazole-4-carboxamide

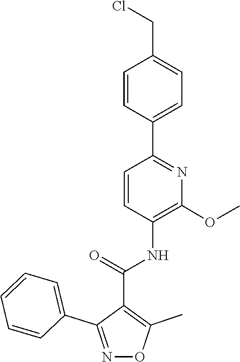 [SMILES: COc1nc(-c2ccc(CCl)cc2)ccc1NC(=O)c1c(-c2ccccc2)noc1C]

To a stirred solution of Example 219 (300.0 mg, 0.72 mmol) in DCM (20 mL) at 0° C. was added thionyl chloride (0.11 mL, 1.44 mmol). The reaction was stirred at 0° C. for 3 hours. The mixture was diluted with saturated sodium bicarbonate and extracted with DCM. The organic phase was dried over anhydrous sodium sulfate, filtered and concentrated under reduced pressure to afford N-[6-[4-(chloromethyl)phenyl]-2-methoxy-3-pyridyl]-5-methyl-3-phenyl-isoxazole-4-carboxamide (290 mg, 88% yield) as a pale brown solid. ¹H NMR (500 MHz, Chloroform-d) 8.64 (d, J=8.2 Hz, 1H), 7.91-7.85 (m, 2H), 7.70 (s, 1H), 7.60-7.49 (m, 5H), 7.37 (d, J=8.2 Hz, 2H), 7.28 (d, J=8.2 Hz, 1H), 4.55 (s, 2H), 3.64 (s, 3H), 2.77 (s, 3H). m/z 434.2 [M+H]⁺.

### Example 225 N-[2-methoxy-6-[4-(morpholinomethyl)phenyl]-3-pyridyl]-5-methyl-3-phenyl-isoxazole-4-carboxamide

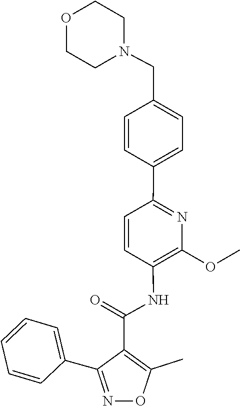 [SMILES: COc1nc(-c2ccc(CN3CCOCC3)cc2)ccc1NC(=O)c1c(-c2ccccc2)noc1C]

Example 224 (70.0 mg, 0.16 mmol), potassium carbonate (44.6 mg, 0.32 mmol), potassium iodide (53.9 mg, 0.32 mmol) and morpholine (0.02 mL, 0.19 mmol) were suspended in DMF (5 mL). The reaction was stirred at 45° C. overnight. The mixture was diluted with DCM (10 mL) and extracted three times with saturated sodium bicarbonate (3×20 mL). The organic layer was dried over sodium sulfate and concentrated under reduced pressure. The crude was purified by flash column chromatography (eluting with 0-10% Methanol in DCM gradient) to afford N-[2-methoxy-6-[4-(morpholinomethyl)phenyl]-3-pyridyl]-5-methyl-3-phenyl-isoxazole-4-carboxamide (52 mg, 63% yield) as an orange solid. ¹H NMR (500 MHZ, CDCl₃) δ8.63 (d, J=8.2 Hz, 1H), 7.89-7.80 (m, 2H), 7.69 (s, 1H), 7.59-7.54 (m, 3H), 7.53-7.48 (m, 2H), 7.33 (s, 2H), 7.26 (d, J=8.2 Hz, 1H), 3.72 (d, J=62.7 Hz, 4H), 3.64-3.63 (m, 3H), 3.47 (s, 2H), 2.80-2.75 (m, 3H), 2.48 (s, 4H). m/z 485.2 [M+H]⁺.

### Example 226 N-[2-methoxy-6-[4-(2-oxa-6-azaspiro[3.3] heptan-6-ylmethyl)phenyl]-3-pyridyl]-5-methyl-3-phenyl-isoxazole-4-carboxamide

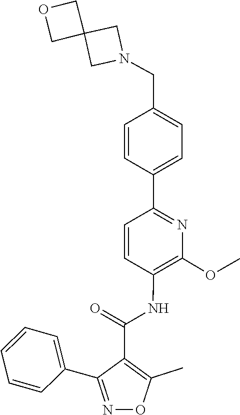 [SMILES: COc1nc(-c2ccc(CN3CC4(COC4)C3)cc2)ccc1NC(=O)c1c(-c2ccccc2)noc1C]

Synthesised using a method similar to Example 225, substituting morpholine with 2-oxa-6-azaspiro[3.3] heptane. Afforded N-[2-methoxy-6-[4-(2-oxa-6-azaspiro[3.3] heptan-6-ylmethyl)phenyl]-3-pyridyl]-5-methyl-3-phenyl-isoxazole-4-carboxamide (72 mg, 60% yield) as a pale orange solid. ¹H NMR (500 MHZ, CDCl₃) δ8.71 (d, J=8.2 Hz, 1H), 7.95-7.90 (m, 2H), 7.78 (s, 1H), 7.69-7.63 (m, 3H), 7.62-7.58 (m, 2H), 7.37-7.30 (m, 3H), 4.77 (s, 4H), 3.72 (s, 3H), 3.60 (s, 2H), 3.43 (s, 4H), 2.86 (s, 3H). m/z 497.3 [M+H]⁺.

### Example 227 N-(2-isopropoxy-6-(isoxazol-4-yl)pyridin-3-yl)-5-methyl-3-phenyl-isoxazole-4-carboxamide

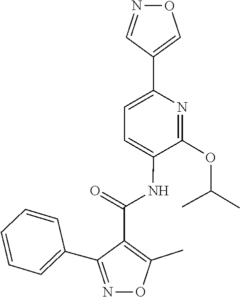 [SMILES: Cc1onc(-c2ccccc2)c1C(=O)Nc1ccc(-c2cnoc2)nc1OC(C)C]

A microwave vial was charged with potassium fluoride (64.0 mg, 1.08 mmol), bis[2-(di-tert-butylphosphanyl)cyclopenta-2,4-dien-1-yl] iron; dichloropalladium (25.0 mg, 0.04 mmol), and N-(6-bromo-2-isopropoxy-3-pyridyl)-5-methyl-3-phenyl-isoxazole-4-carboxamide (see Example 206, step 1) (150.0 mg, 0.36 mmol). The mixture was solvated with 1,4-dioxane (3 mL) and Water (0.3 mL) degassed with nitrogen for 10 minutes prior to the addition of 4-Isoxazoleboronic acid pinacol ester (72.0 mg, 0.37 mmol). The mixture was re-purged with nitrogen for 5 minutes then heated to 50° C. overnight. Solvent was removed by rotary evaporation. The resulting crude brown solid was suspended in water and filtered, washing on the sinter with additional water. The solid was subjected to silica gel column chromatography (Pet ether:DCM, 1:0→0:1); removal of solvent from selected fractions resulted in the isolation of the anticipated product as a white solid, which was triturated in MeOH prior to final analysis as N-(2-isopropoxy-6-(isoxazol-4-yl)pyridin-3-yl)-5-methyl-3-phenylisoxazole-4-carboxamide. ¹H NMR (500 MHZ, Chloroform-d) δ8.81 (s, 1H), 8.74 (d, J=8.1 Hz, 1H), 8.67 (s, 1H), 7.76 (s, 1H), 7.72-7.65 (m, 2H), 7.63-7.52 (m, 2H), 7.07 (d, J=8.1 Hz, 1H), 5.23 (hept, J=6.0 Hz, 1H), 2.82 (s, 3H), 1.06 (d, J=6.2 Hz, 6H). 405.2 [M+H]⁺.

### Example 228 N-[2-methoxy-6-[4-(3-oxa-7-azabicyclo[3.3.1] nonan-7-ylmethyl)phenyl]-3-pyridyl]-5-methyl-3-phenyl-isoxazole-4-carboxamide

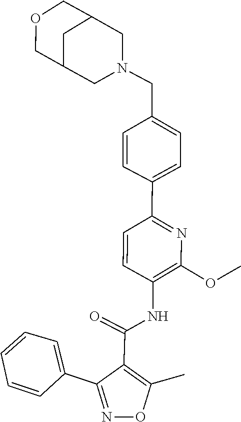 [SMILES: COc1nc(-c2ccc(CN3CC4COCC(C4)C3)cc2)ccc1NC(=O)c1c(-c2ccccc2)noc1C]

Synthesised using a method similar to Example 225, substituting morpholine with 3-oxa-7-azabicyclo[3.3.1] nonane. Afforded N-[2-methoxy-6-[4-(3-oxa-7-azabicyclo[3.3.1] nonan-7-ylmethyl)phenyl]-3-pyridyl]-5-methyl-3-phenyl-isoxazole-4-carboxamide (70 mg, 55% yield) as a colourless solid. ¹H NMR (500 MHZ, CDCl₃) δ8.71 (d, J=8.2 Hz, 1H), 8.00-7.87 (m, 2H), 7.78 (s, 1H), 7.69-7.63 (m, 3H), 7.60 (dd, J=8.0, 6.4 Hz, 2H), 7.47 (s, 2H), 7.36 (d, J=8.2 Hz, 1H), 4.08-3.90 (m, 2H), 3.80 (d, J=10.8 Hz, 2H), 3.73 (s, 3H), 3.57 (d, J=11.0 Hz, 2H), 3.00 (d, J=11.4 Hz, 2H), 2.86 (s, 3H), 2.62-2.22 (m, 2H), 1.89-1.58 (m, 4H). m/z 525.3 [M+H]⁺.

### Example 229 N-(2-isoxazol-4-yl-4-methoxy-pyrimidin-5-yl)-5-methyl-3-phenyl-isoxazole-4-carboxamide

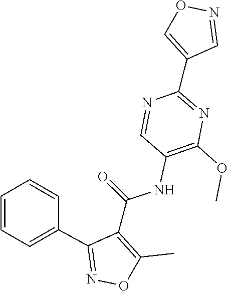 [SMILES: COc1nc(-c2cnoc2)ncc1NC(=O)c1c(-c2ccccc2)noc1C]

A microwave vial was charged with 4-Isoxazoleboronic acid pinacol ester (75 mg, 0.38 mmol), N-(2-chloro-4-methoxy-pyrimidin-5-yl)-5-methyl-3-phenyl-isoxazole-4-carboxamide (125 mg, 0.36 mmol) (intermediate 3 from Example 3), 1,4-Dioxane (2.9 mL) and water (0.70 mL) and sealed. The mixture was purged with nitrogen for 5 minutes prior to the addition of fluoride potassium (65 mg, 1.1 mmol) and bis[2-(di-tert-butylphosphanyl)cyclopenta-2,4-dien-1-yl] iron; dichloropalladium (Pd 118) (12 mg, 0.02 mmol). The mixture was resealed and purged with nitrogen for five minutes prior to heating to 90° C. overnight. The cooled reaction mixture was quenched with water and extracted with DCM (2×). The combined organic extracts were washed with brine, dried over sodium sulfate, filtered, and the solvent removed by rotary evaporation. The crude material thus isolated was subjected to silica gel column chromatography (eluent DCM:MeOH, 1:0→95:5). Material thus isolated was re-subjected to silica gel column chromatography (eluent, petrol ether:EtOAc:DCM:MeOH, 1:0:0:0→0:1:0:0→0:0:1:0→0:0:95:5). Fractions identified as containing the anticipated product were concentrated by rotary evaporation and the material thus isolated triturated in MeOH and filtered to deliver the anticipated product N-(2-isoxazol-4-yl-4-methoxy-pyrimidin-5-yl)-5-methyl-3-phenyl-isoxazole-4-carboxamide (18 mg, 0.0477 mmol, 13.156% yield) as a brown solid. ¹H NMR (500 MHZ, DMSO-d₆) δ9.95 (s, 1H), 9.13 (s, 1H), 8.93 (s, 1H), 7.70-7.64 (m, 2H), 7.59-7.50 (m, 4H), 4.01 (s, 3H), 2.63 (s, 3H). m/z 378.2 [M+H]⁺.

### Example 230 N-(6-methoxy-6′-(morpholinomethyl)-[2,3′-bipyridin]-5-yl)-5-methyl-3-phenyl-isoxazole-4-carboxamide

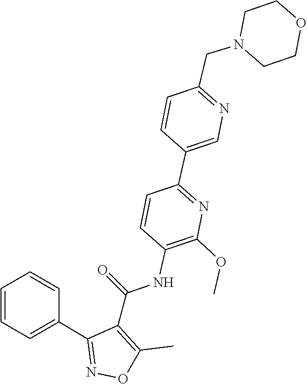 [SMILES: COc1nc(-c2ccc(CN3CCOCC3)nc2)ccc1NC(=O)c1c(-c2ccccc2)noc1C]

### Step 1-Synthesis of Example 27-N-(6′-(hydroxymethyl)-6-methoxy-[2,3′-bipyridin]-5-yl)-5-methyl-3-phenylisoxazole-4-carboxamide

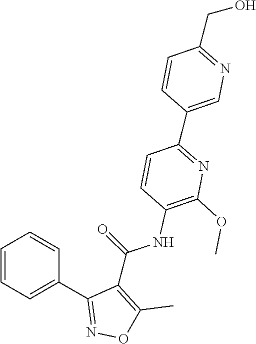 [SMILES: COc1nc(-c2ccc(CO)nc2)ccc1NC(=O)c1c(-c2ccccc2)noc1C]

A suspension of N-[2-methoxy-6-(4,4,5,5-tetramethyl-1,3,2-dioxaborolan-2-yl)-3-pyridyl]-5-methyl-3-phenyl-isoxazole-4-carboxamide (Intermediate 7) (1500 mg, 3.45 mmol), (5-bromopyrid-2-yl) methanol (777.51 mg, 4.14 mmol) and POTASSIUM FLUORIDE (611.03 mg, 10.34 mmol) in Dioxane/water (24:6 mL) was purged with nitrogen gas for 5 minutes. bis[2-(di-tert-butylphosphanyl)cyclopenta-2,4-dien-1-yl] iron dichloropalladium (225.29 mg, 0.34 mmol) was then added and the mixture purged with nitrogen for an additional 5 minutes. The vial was sealed and the suspension was stirred overnight at 45° C. The solution was diluted with EtOAc (100 mL) and washed with water (2×100 mL); the organic phase was dried over sodium sulfate and concentrated under vacuum. The resulting residue was purified by flash chromatography using an ISCO system (24 g silica, 0-5% MeOH in DCM gradient). Fractions containing product were combined and concentrated to give N-(6′-(hydroxymethyl)-6-methoxy-[2,3′-bipyridin]-5-yl)-5-methyl-3-phenylisoxazole-4-carboxamide (Example 27) (510 mg, 1.1635 mmol, 33.762% yield) as a pale brown solid. 1H NMR (500 MHZ, CDCl₃) δ9.26-9.08 (m, 1H), 8.77 (d, J=8.1 Hz, 1H), 8.34-8.27 (m, 1H), 7.81 (s, 1H), 7.69-7.64 (m, 3H), 7.63-7.59 (m, 2H), 7.38 (d, J=8.1 Hz, 2H), 4.86 (s, 2H), 3.73 (s, 3H), 2.86 (s, 3H). ACQUITY UPLC® BEH C18 1.7 μm: Rt=1.74 min; m/z 417.3 [M+H]⁺.

### Step 2—Synthesis of Intermediate 16-N-(6′-(chloromethyl)-6-methoxy-[2,3′-bipyridin]-5-yl)-5-methyl-3-phenylisoxazole-4-carboxamide

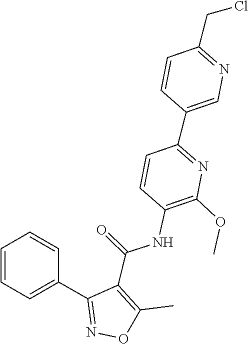 [SMILES: COc1nc(-c2ccc(CCl)nc2)ccc1NC(=O)c1c(-c2ccccc2)noc1C]

To a stirred solution of N-(6′-(hydroxymethyl)-6-methoxy-[2,3′-bipyridin]-5-yl)-5-methyl-3-phenylisoxazole-4-carboxamide (Example 27) (500 mg, 1.2 mmol) in DCM (20 mL), was added thionyl chloride (0.18 mL, 2.4 mmol) at 0° C. The reaction was stirred for 2 hours at 0° C. The mixture was diluted with DCM (20 mL) and washed three times with saturated sodium bicarbonate (3×20 mL). The organic phase was dried over sodium sulfate, filtered and concentrated under reduced pressure to yield N-(6′-(chloromethyl)-6-methoxy-[2,3′-bipyridin]-5-yl)-5-methyl-3-phenylisoxazole-4-carboxamide (Intermediate 16) (430 mg, 0.9394 mmol, 78.236% yield) as a pale brown solid. ¹H NMR (500 MHZ, CDCl₃) δ9.09 (t, J=6.8 Hz, 1H), 8.69 (t, J=7.0 Hz, 1H), 8.20 (t, J=7.0 Hz, 1H), 7.73 (t, J=6.9 Hz, 1H), 7.63-7.41 (m, 6H), 7.36-7.25 (m, 1H), 4.79-4.54 (m, 2H), 3.76-3.52 (m, 3H), 2.91-2.71 (m, 3H). ACQUITY UPLC® BEH C18 1.7 μm: Rt=1.96 min; m/z 433.2 [M+H]⁺.

### Step 3

N-(6′-(chloromethyl)-6-methoxy-[2,3′-bipyridin]-5-yl)-5-methyl-3-phenylisoxazole-4-carboxamide (Intermediate 16) (100 mg, 0.23 mmol), morpholine (0.02 mL, 0.28 mmol), potassium carbonate (64.49 mg, 0.46 mmol) and potassium iodide (76.81 mg, 0.46 mmol) were suspended in DMF (5 mL) and the reaction stirred at 45° C. for 3 hours. The mixture was diluted with DCM (20 mL) and washed with saturated sodium bicarbonate (3×20 mL). The organic layer was dried over sodium sulfate and concentrated under vacuum. The crude was purified by ISCO flash column chromatography (4 g silica, 0-5% Methanol in DCM gradient). Fractions containing product were combined and concentrated to N-(6-methoxy-6′-(morpholinomethyl)-[2,3′-bipyridin]-5-yl)-5-methyl-3-phenylisoxazole-4-carboxamide (95 mg, 0.1917 mmol, 83.386% yield) as a pale orange solid. ¹H NMR (500 MHz, DMSO) δ9.49 (s, 1H), 9.17 (d, J=2.3 Hz, 1H), 8.38 (dd, J=8.2, 2.4 Hz, 2H), 7.70 (dd, J=26.2, 7.5 Hz, 3H), 7.60-7.49 (m, 4H), 3.92 (s, 3H), 3.65 (s, 2H), 3.61 (t, J=4.7 Hz, 4H), 2.67 (s, 3H), 2.44 (s, 4H). ACQUITY UPLC® BEH C18 1.7 μm: Rt=1.75 min; m/z 486.3 [M+H]⁺.

### Example 231 N-(6′-((2-oxa-6-azaspiro[3.3] heptan-6-yl)methyl)-6-methoxy-[2,3′-bipyridin]-5-yl)-5-methyl-3-phenylisoxazole-4-carboxamide

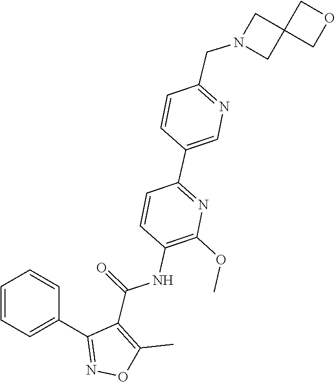 [SMILES: COc1nc(-c2ccc(CN3CC4(COC4)C3)nc2)ccc1NC(=O)c1c(-c2ccccc2)noc1C]

N-(6′-(chloromethyl)-6-methoxy-[2,3′-bipyridin]-5-yl)-5-methyl-3-phenylisoxazole-4-carboxamide (Intermediate 16) (100 mg, 0.23 mmol), potassium carbonate (64.49 mg, 0.46 mmol), potassium iodide (76.81 mg, 0.46 mmol) and 2-oxa-6-azaspiro[3.3] heptane (27.35 mg, 0.28 mmol) were suspended in DMF (5 mL).

The reaction was stirred at 45° C. for 2 hours. The mixture was diluted with DCM (20 mL) and washed with saturated sodium bicarbonate (3×20 mL). Combined organics were dried over sodium sulfate and concentrated under vacuum. The crude was purified by flash column chromatography using an ISCO system (4 g silica, 0-5% Methanol in DCM gradient). Fractions containing product were combined and concentrated to give N-(6′-((2-oxa-6-azaspiro[3.3] heptan-6-yl)methyl)-6-methoxy-[2,3′-bipyridin]-5-yl)-5-methyl-3-phenylisoxazole-4-carboxamide (88 mg, 0.1733 mmol, 75.376% yield) as an orange solid. 1H NMR (500 MHZ, DMSO) δ9.49 (s, 1H), 9.14 (dd, J=2.3, 0.8 Hz, 1H), 8.50-8.26 (m, 2H), 7.69 (dd, J=29.8, 7.5 Hz, 3H), 7.61-7.49 (m, 3H), 7.45-7.39 (m, 1H), 4.63 (s, 4H), 3.92 (s, 3H), 3.67 (s, 2H), 3.40 (s, 4H), 2.67 (s, 3H). ACQUITY UPLC® BEH C18 1.7 μm: Rt=1.71 min; m/z 498.3 [M+H]⁺.

### Example 232 N-(6′-((3-oxa-7-azabicyclo[3.3.1] nonan-7-yl)methyl)-6-methoxy-[2,3′-bipyridin]-5-yl)-5-methyl-3-phenylisoxazole-4-carboxamide

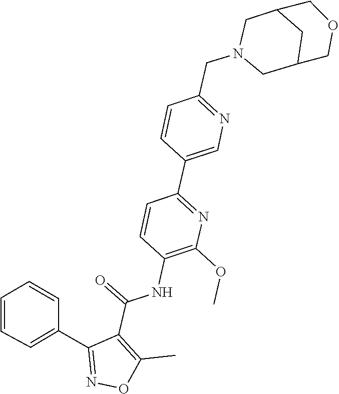 [SMILES: COc1nc(-c2ccc(CN3CC4COCC(C4)C3)nc2)ccc1NC(=O)c1c(-c2ccccc2)noc1C]

N-(6′-(chloromethyl)-6-methoxy-[2,3′-bipyridin]-5-yl)-5-methyl-3-phenylisoxazole-4-carboxamide (Intermediate 16) (100 mg, 0.23 mmol), potassium carbonate (64.49 mg, 0.46 mmol), potassium iodide (76.81 mg, 0.46 mmol) and 3-oxa-7-azabicyclo[3.3.1] nonane (35.09 mg, 0.28 mmol) were suspended in DMF (5 mL).

The reaction was stirred at 45° C. for 2 hours. The mixture was diluted with DCM (20 mL) and washed with saturated sodium bicarbonate (3×20 mL). The organic layer was dried over sodium sulfate and concentrated under vacuum. The crude was purified by ISCO flash column chromatography (4 g silica, 0-5% Methanol in DCM gradient). Fractions containing product were combined and concentrated to give N-(6′-((3-oxa-7-azabicyclo[3.3.1] nonan-7-yl)methyl)-6-methoxy-[2,3′-bipyridin]-5-yl)-5-methyl-3-phenylisoxazole-4-carboxamide (77 mg, 0.1436 mmol, 62.434% yield) as an off white solid. ¹H NMR (500 MHZ, DMSO) δ 9.48 (s, 1H), 9.15 (d, J=2.2 Hz, 1H), 8.40 (dd, J=8.2, 2.3 Hz, 2H), 7.72 (d, J=7.0 Hz, 2H), 7.67 (d, J=8.2 Hz, 2H), 7.60-7.54 (m, 3H), 3.92 (s, 3H), 3.82 (d, J=10.9 Hz, 2H), 3.68 (dt, J=11.1, 2.3 Hz, 2H), 3.63-3.54 (m, 2H), 2.94 (d, J=10.7 Hz, 2H), 2.68 (s, 3H), 2.41 (d, J=10.8 Hz, 2H), 1.78 (d, J=12.0 Hz, 1H), 1.72 (s, 2H), 1.57 (d, J=12.1 Hz, 1H). ACQUITY UPLC® BEH C18 1.7 μm: Rt=1.85 min; m/z 526.3 [M+H]⁺.

### Example 233 N-(2-methoxy-6-(4-((tetrahydro-1H-furo[3,4-c]pyrrol-5 (3H)-yl)methyl)phenyl)pyridin-3-yl)-5-methyl-3-phenylisoxazole-4-carboxamide

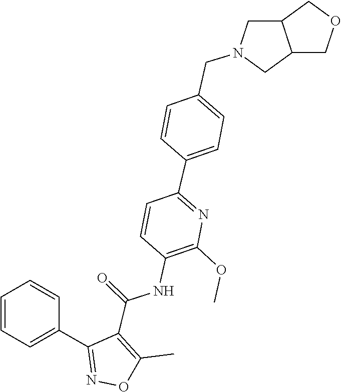 [SMILES: COc1nc(-c2ccc(CN3CC4COCC4C3)cc2)ccc1NC(=O)c1c(-c2ccccc2)noc1C]

N-(6-(4-(chloromethyl)phenyl)-2-methoxypyridin-3-yl)-5-methyl-3-phenylisoxazole-4-carboxamide (Example 224) (100 mg, 0.23 mmol), potassium carbonate (96.95 mg, 0.69 mmol), potassium iodide (76.98 mg, 0.46 mmol) and Hexahydro-1H-furo[3,4-c]pyrrole hydrochloride (41.38 mg, 0.28 mmol) were suspended in DMF (5 mL) and the reaction was stirred at 45° C. for 3 hours. The mixture was diluted with DCM (15 mL) and washed with saturated sodium bicarbonate (3×20 mL). The organic phase was dried over sodium sulfate and concentrated under reduced pressure. The crude was purified by ISCO flash column chromatography (4 g silica, 0-5% Methanol in DCM gradient). Fractions containing product were combined and concentrated to give N-(2-methoxy-6-(4-((tetrahydro-1H-furo[3,4-c] pyrrol-5 (3H)-yl)methyl)phenyl)pyridin-3-yl)-5-methyl-3-phenylisoxazole-4-carboxamide (75 mg, 0.1395 mmol, 60.548% yield) as an orange solid. ¹H NMR (500 MHZ, DMSO) δ9.45 (s, 1H), 8.35 (d, J=8.2 Hz, 1H), 8.02 (d, J=7.8 Hz, 2H), 7.78-7.70 (m, 2H), 7.57 (dtd, J=7.4, 5.4, 5.0, 3.0 Hz, 4H), 7.40 (d, J=7.9 Hz, 2H), 3.91 (s, 3H), 3.72 (d, J=7.2 Hz, 2H), 3.59 (s, 2H), 3.48-3.36 (m, 2H), 2.72 (s, 2H), 2.67 (s, 3H), 2.54 (d, J=8.6 Hz, 2H), 2.43-2.24 (m, 2H). ACQUITY UPLC® BEH C18 1.7 μm: Rt=1.67 min; m/z 511.3 [M+H]⁺.

### Biological Data

The compounds shown in Table 1 exhibited the following activity in the in vitro radioligand binding assay and in vitro α5-GABA_{A}R Relative Efficacy assays described herein:

TABLE 1

| | | α5-GABA_{A}R Relative | | α5-GABA_{A}R Relative | |
|---|---|---|---|---|---|
| | | Efficacy (to DMCM), | | Efficacy (to DMCM), | |
| | α5- | Qpatch | | SyncroPatch | |
| | GABA_{A}R | Relative | Conc | Relative | Conc |
| Example | Kᵢ (nM) | Efficacy | (μM) | Efficacy | (μM) |
| Example 1 | 2.9 | −0.91 | 1 | −1.11 | 0.1 |
| Example 2 | 2.4 | −0.54 | 1 | | |
| Example 3 | 32.8 | −0.69 | 1 | | |
| Example 4 | 29.7 | −0.68 | 1 | −0.36 | 0.1 |
| Example 5 | 50.7 | −0.82 | 1 | | |
| Example 6 | 33.1 | −0.22 | 10 | | |
| Example 7 | 2.0 | −0.77 | 0.3 | | |
| Example 8 | 103.7 | | | | |
| Example 9 | 3.2 | −0.21 | 1 | | |
| Example 10 | 72.9 | | | | |
| Example 11 | 2.9 | −0.84 | 0.3 | | |
| Example 12 | 17.1 | −0.68 | 3 | | |
| Example 13 | 3.6 | −0.85 | 1 | | |
| Example 14 | 50.0 | −0.24 | 10 | | |
| Example 15 | 7.0 | −0.19 | 1 | | |
| Example 16 | 403.3 | | | | |
| Example 17 | 10.8 | −0.81 | 3 | | |
| Example 18 | 2.8 | −0.61 | 0.3 | | |
| Example 19 | 4.3 | −0.86 | 1 | | |
| Example 20 | 13.9 | −0.28 | 3 | | |
| Example 21 | 20.5 | −0.48 | 3 | | |
| Example 22 | 15.8 | 0.15 | 3 | | |
| Example 23 | 2.4 | −0.46 | 0.3 | | |
| Example 24 | 161.7 | | | | |
| Example 25 | 34.2 | | | | |
| Example 26 | 8.0 | −0.68 | 1 | | |
| Example 27 | 2.3 | −0.83 | 0.3 | | |
| Example 28 | 5.2 | −0.61 | 1 | | |
| Example 29 | 1.5 | −0.53 | 0.3 | | |
| Example 30 | 5.1 | −0.42 | 1 | | |
| Example 31 | 69.1 | | | | |
| Example 32 | 110.0 | | | | |
| Example 33 | 15.8 | −0.50 | 3 | | |
| Example 34 | 115.3 | | | | |
| Example 35 | 27.2 | | | | |
| Example 36 | 2.0 | −0.86 | 0.3 | | |
| Example 37 | 2.8 | −0.56 | 0.3 | | |
| Example 38 | 57.6 | | | | |
| Example 39 | 32.3 | | | | |
| Example 40 | 6.9 | −0.57 | 1 | | |
| Example 41 | 1.8 | −0.87 | 0.3 | | |
| Example 42 | 2.8 | −0.60 | 0.3 | | |
| Example 43 | 0.5 | −0.76 | 0.1 | −0.90 | 0.1 |
| Example 44 | 3.6 | −0.69 | 1 | | |
| Example 45 | 1.2 | −0.65 | 0.3 | | |
| Example 46 | 4.4 | −0.69 | 1 | | |
| Example 47 | 0.8 | −0.81 | 0.1 | −1.00 | 0.1 |
| Example 48 | 2.7 | −0.90 | 0.3 | | |
| Example 49 | 7.9 | −0.83 | 1 | | |
| Example 50 | 9.6 | −0.74 | 1 | | |
| Example 51 | 20.5 | | | | |
| Example 52 | 9.8 | −0.49 | 1 | | |
| Example 53 | 2.4 | −0.92 | 0.3 | −0.83 | 0.1 |
| Example 54 | 7.4 | −0.55 | 1 | | |
| Example 55 | 3.4 | −0.92 | 1 | −0.93 | 0.1 |
| Example 56 | 2.3 | −0.64 | 0.3 | | |
| Example 57 | 1.5 | −0.57 | 0.3 | | |
| Example 58 | 18.1 | −0.79 | 3 | | |
| Example 59 | 10.4 | −0.75 | 3 | | |
| Example 60 | 1.3 | −0.92 | 0.3 | | |
| Example 61 | 1.4 | −0.85 | 0.3 | | |
| Example 62 | 1.9 | −0.61 | 0.3 | | |
| Example 63 | 32.6 | | | | |
| Example 64 | 21.9 | | | | |
| Example 65 | 49.0 | | | | |
| Example 66 | 235.6 | | | | |
| Example 67 | 0.6 | −0.94 | 0.1 | −1.14 | 0.1 |
| Example 68 | 3.2 | −0.66 | 1 | −0.56 | 0.1 |
| Example 69 | 232.0 | | | | |
| Example 70 | 0.7 | −0.89 | 0.1 | | |
| Example 71 | 2.8 | −0.94 | 0.3 | | |
| Example 72 | 2.0 | −1.04 | 0.3 | | |
| Example 73 | 153.8 | | | | |
| Example 74 | 2.1 | −1.00 | 0.3 | −0.62 | 0.1 |
| Example 75 | 0.6 | −0.92 | 0.1 | | |
| Example 76 | 296.9 | | | | |
| Example 77 | 16.1 | −0.22 | 3 | | |
| Example 78 | 1.0 | −0.42 | 0.1 | | |
| Example 79 | 0.8 | −0.76 | 0.1 | | |
| Example 80 | 19.5 | −0.65 | 3 | | |
| Example 81 | 8.0 | −1.26 | 1 | | |
| Example 82 | 7.9 | −0.65 | 1 | | |
| Example 83 | 13.0 | −0.71 | 3 | −0.50 | 0.1 |
| Example 84 | 10.7 | −0.51 | 3 | | |
| Example 85 | 1.7 | −0.92 | 0.3 | −0.94 | 0.1 |
| Example 86 | 3.2 | −0.65 | 1 | | |
| Example 87 | 5.0 | −0.71 | 1 | −0.44 | 0.1 |
| Example 88 | 26.6 | | | | |
| Example 89 | 3.1 | −0.64 | 1 | −0.62 | 0.1 |
| Example 90 | 3.4 | −0.58 | 1 | | |
| Example 91 | 10.1 | −0.45 | 3 | | |
| Example 92 | 3.0 | −0.66 | 0.3 | | |
| Example 93 | 8.2 | −0.73 | 1 | | |
| Example 94 | 21.7 | −0.50 | 3 | | |
| Example 95 | 1.4 | −0.66 | 0.3 | | |
| Example 96 | 1.9 | −0.52 | 0.3 | | |
| Example 97 | 19.1 | −0.67 | 3 | | |
| Example 98 | 663.7 | | | | |
| Example 99 | 6.7 | −0.70 | 1 | | |
| Example 100 | 3.0 | −0.72 | 0.3 | | |
| Example 101 | 45.1 | | | | |
| Example 102 | 1.5 | −0.79 | 0.3 | | |
| Example 103 | 6.6 | −0.78 | 1 | | |
| Example 104 | 1.7 | −0.68 | 0.3 | | |
| Example 105 | 112.8 | | | | |
| Example 106 | 11.4 | −0.40 | 3 | | |
| Example 107 | 13.5 | −0.66 | 3 | −0.47 | 0.1 |
| Example 108 | 3.1 | −0.81 | 1 | | |
| Example 109 | 47.2 | | | | |
| Example 110 | 29.1 | −0.53 | 3 | | |
| Example 111 | 13.1 | −0.46 | 3 | | |
| Example 112 | 80.6 | | | | |
| Example 113 | 41.7 | | | | |
| Example 114 | 6.3 | −0.74 | 1 | | |
| Example 115 | 99.8 | | | | |
| Example 116 | 2.9 | −0.84 | 0.3 | | |
| Example 117 | 2.6 | −0.70 | 0.3 | | |
| Example 118 | 136.9 | | | | |
| Example 119 | 13.8 | −0.63 | 3 | | |
| Example 120 | 4.6 | −0.73 | 1 | | |
| Example 121 | 2.1 | −0.36 | 0.3 | | |
| Example 122 | 125.5 | | | | |
| Example 123 | 11.8 | −0.48 | 3 | | |
| Example 124 | 23.6 | −1.40 | 3 | | |
| Example 125 | 2.5 | −0.84 | 0.3 | | |
| Example 126 | 1.1 | −0.68 | 0.3 | −0.55 | 0.1 |
| Example 127 | 21.8 | −0.72 | 3 | −0.57 | 0.1 |
| Example 128 | 46.7 | −0.19 | 10 | | |
| Example 129 | 0.8 | −0.77 | 0.1 | −0.72 | 0.1 |
| Example 130 | 2.3 | −0.55 | 0.3 | | |
| Example 131 | 9.8 | −0.67 | 1 | | |
| Example 132 | 4.0 | −0.52 | 1 | | |
| Example 133 | 4.1 | −0.75 | 1 | | |
| Example 134 | 51.9 | | | | |
| Example 135 | 1.0 | −0.85 | 0.3 | | |
| Example 136 | 3.3 | −0.60 | 1 | | |
| Example 137 | 3.8 | −0.80 | 1 | −0.69 | 0.1 |
| Example 138 | 3.2 | −0.62 | 1 | | |
| Example 139 | 1.9 | −0.90 | 0.3 | | |
| Example 140 | 5.7 | −0.79 | 1 | | |
| Example 141 | 1.9 | −0.93 | 0.3 | −1.13 | 0.1 |
| Example 142 | 2.9 | −0.85 | 0.3 | | |
| Example 143 | 0.4 | −0.80 | 0.1 | −0.72 | 0.1 |
| Example 144 | 1.4 | −0.95 | 0.3 | −1.05 | 0.1 |
| Example 145 | 0.3 | −1.12 | 0.1 | | |
| Example 146 | 79.0 | | | | |
| Example 147 | 0.5 | −0.67 | 0.1 | | |
| Example 148 | 1.2 | −0.80 | 0.3 | | |
| Example 149 | 5.2 | −0.66 | 1 | | |
| Example 150 | 5.1 | −0.83 | 1 | | |
| Example 151 | 45.1 | −0.89 | 3 | | |
| Example 152 | 22.9 | −0.27 | 3 | | |
| Example 153 | 1.7 | −0.83 | 0.3 | | |
| Example 154 | 25.3 | −0.89 | 3 | | |
| Example 155 | 2.3 | −0.91 | 0.3 | | |
| Example 156 | 2.7 | −0.91 | 0.3 | −0.65 | 0.1 |
| Example 157 | 45.8 | | | | |
| Example 158 | 8.4 | | | | |
| Example 159 | 9.2 | −0.82 | 1 | −0.81 | 0.1 |
| Example 160 | 28.0 | −0.77 | 3 | | |
| Example 161 | 61.5 | −0.71 | 10 | | |
| Example 162 | 33.6 | −0.90 | 3 | | |
| Example 163 | 4.1 | −1.11 | 1 | | |
| Example 164 | 27.2 | −0.65 | 3 | | |
| Example 165 | 35.4 | −0.84 | 3 | | |
| Example 166 | 31.0 | −0.98 | 3 | | |
| Example 167 | 0.7 | −0.92 | 1 | | |
| Example 168 | 2.7 | −0.72 | 0.3 | | |
| Example 169 | 5.2 | −0.88 | 1 | | |
| Example 170 | 0.5 | −0.84 | 0.1 | | |
| Example 171 | 3.8 | −0.75 | 0.1 | | |
| Example 172 | 0.2 | −0.80 | 0.1 | | |
| Example 173 | 0.3 | −0.88 | 0.1 | | |
| Example 174 | 1.4 | −0.84 | 0.3 | | |
| Example 175 | 0.5 | −0.89 | 0.1 | | |
| Example 176 | 9.0 | −0.36 | 1 | | |
| Example 177 | 544.9 | | | | |
| Example 178 | 2.0 | −0.62 | 0.3 | | |
| Example 179 | 3.0 | −0.65 | 0.3 | −0.38 | 0.1 |
| Example 180 | 3.9 | −0.63 | 1 | −0.48 | 0.1 |
| Example 181 | 1.9 | | | −1.05 | 0.1 |
| Example 182 | 8.8 | −0.53 | 1 | −0.17 | 0.1 |
| Example 183 | 14.2 | −0.68 | 3 | | |
| Example 184 | 1.5 | −0.45 | 0.3 | | |
| Example 185 | 0.5 | −0.79 | 0.1 | −1.23 | 0.1 |
| Example 186 | 17.5 | −0.69 | 3 | | |
| Example 187 | 23.5 | −0.46 | 3 | | |
| Example 188 | 0.5 | −1.08 | 0.1 | | |
| Example 189 | 0.7 | −0.70 | 0.1 | −0.72 | 0.1 |
| Example 190 | 1.1 | −0.59 | 0.1 | −0.72 | 0.1 |
| Example 191 | 1.3 | | | | |
| Example 192 | 1.2 | | | | |
| Example 193 | 22.9 | | | | |
| Example 194 | 38.7 | | | | |
| Example 195 | 23.5 | | | | |
| Example 196 | 23.5 | −0.71 | 3 | −0.68 | 0.1 |
| Example 197 | 0.4 | | | | |
| Example 198 | 4.3 | | | | |
| Example 199 | 1.6 | −0.82 | 0.3 | −0.93 | 0.1 |
| Example 200 | 16.8 | −0.53 | 3 | | |
| Example 201 | 35.4 | | | | |
| Example 202 | 77.8 | | | | |
| Example 203 | 17.0 | | | | |
| Example 204 | 1.1 | −0.39 | 0.1 | | |
| Example 205 | 0.4 | −0.61 | 0.1 | | |
| Example 206 | 1.7 | −0.55 | 0.3 | −1.03 | 0.1 |
| Example 207 | 0.3 | −0.56 | 0.1 | −0.83 | 0.1 |
| Example 208 | 0.7 | −0.38 | 0.1 | −0.70 | 0.1 |
| Example 209 | 0.3 | | | −0.60 | 0.1 |
| Example 210 | 13.7 | | | | |
| Example 211 | 1.3 | | | | |
| Example 212 | 312.7 | | | | |
| Example 213 | 0.4 | | | −0.85 | 0.1 |
| Example 214 | 0.4 | | | −0.81 | 0.1 |
| Example 215 | 0.6 | | | −0.65 | 0.1 |
| Example 216 | 1.5 | | | −0.67 | 0.1 |
| Example 217 | 6.4 | | | −0.16 | 0.1 |
| Example 218 | 19.5 | | | −0.51 | 0.1 |
| Example 219 | 4.4 | | | −0.45 | 0.03 |
| Example 220 | 3.4 | | | | |
| Example 221 | 0.8 | | | −1.15 | 0.1 |
| Example 222 | 0.5 | | | −0.78 | 0.1 |
| Example 223 | 1.3 | | | −1.06 | 0.1 |
| Example 224 | 220.4 | | | | |
| Example 225 | 7.7 | | | | |
| Example 226 | 3.3 | | | | |
| Example 227 | 0.5 | | | | |
| Example 228 | 1.3 | | | −0.68 | 0.1 |
| Example 229 | 87.4 | | | −0.79 | 0.1 |
| Example 230 | 1.4 | | | −1.10 | 0.1 |
| Example 231 | 1.3 | | | −1.00 | 0.1 |
| Example 232 | 0.6 | | | −0.73 | 0.1 |
| Example 233 | 7 | | | | |

### In Vitro Electrophysiological Recording Assay (α5-GABA_{A}R Relative Efficacy)

Raw whole-cell current traces were recorded on the QPatch system from the same cell expressing α53γ2 GABA_{A} receptors before and after the addition of the compound of Example 1 (1 μM) using the in vitro electrophysiological recording assay described herein. FIG. 1 shows that the same concentration of GABA elicits smaller current in the presence of the compound of Example 1, consistent with the effect expected for a α5-GABA_{A}R NAM.

### Electrophysiology In Hippocampal Slices-Long Term Potentiation Assay

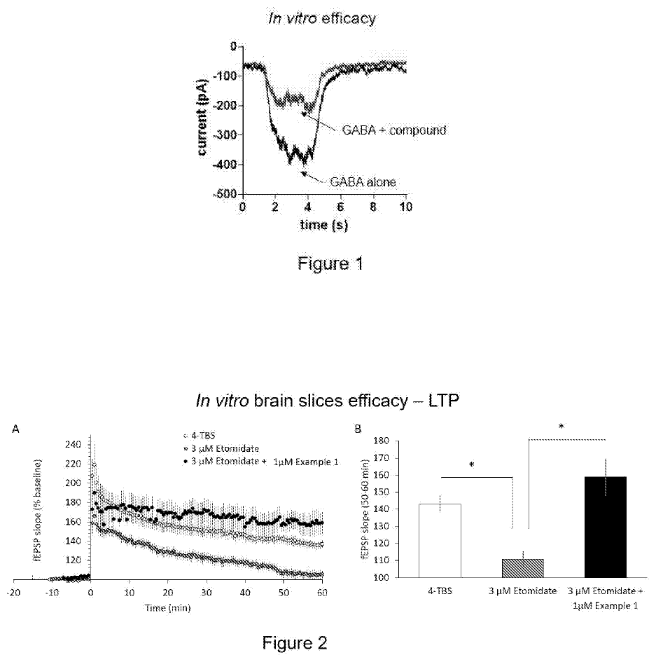

FIG. 2 illustrates the rescue of the etomidate-mediated long-term potentiation (LTP) deficit by compound of Example 1 in mouse hippocampal brain slices.

LTP, which is a measure of increased synaptic efficacy, was induced using a standard paradigm that involves a 4-theta burst stimulation (4-TBS). A plot of the fEPSP slope (expressed as a % of the control fEPSPs prior to the 4-TBS) vs time is shown in FIG. 2A. Following the recording of fEPSPs (1/30 sec), at time zero a 4-TBS was delivered to induce control LTP (n=8). The magnitude of LTP is reduced by etomidate (3 μM; n=17), but is rescued by co-addition of the compound of Example 1 (1 μM; n=6).

The magnitude of LTP determined between 50-60 min post-delivery of the 4-TBS is illustrated in the form a bar chart in FIG. 2B. Etomidate significantly reduced fEPSP slope at 50-60 min compared to control. Co-incubation of slices with the compound of Example 1 significantly increased fEPSP slope (one-way ANOVA with post-hoc Tukey test, p<0.05), such that there is no difference to the magnitude of control LTP.

### In Vivo Brain Receptor Occupancy Assay

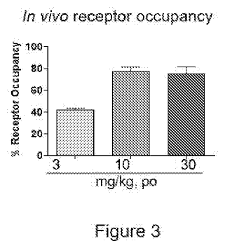

The occupancy of rat brain α5-GABA_{A}Rs binding sites by the compound of Example 1 was assessed in the in vivo brain receptor occupancy assay described herein. For doses of the compound of Example 1 in the range 3 to 30 mg/kg (p.o.), the occupancy of rat brain α5-GABA_{A}Rs (i.e., the inhibition of in vivo [³H] L655,708 binding) was dose-dependent with the dose of 10 mg/kg estimated to occupy 77% of the brain α5 GABA_{A} receptors (FIG. 3).

### Further Embodiments

The invention is further illustrated by the following numbered clauses:
P1. A compound of the formula (I), or a pharmaceutically acceptable salt thereof:

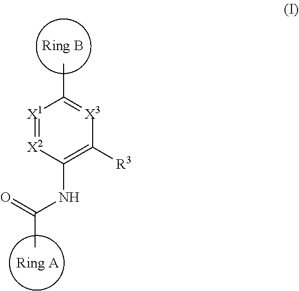

wherein
Ring A is selected from: A1, A2 and A3:

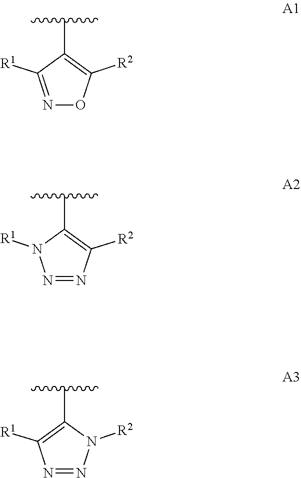

R¹ is selected from: phenyl and a 5- or 6-membered heteroaryl, wherein R¹ is optionally substituted by one or more substituents selected from: halo, C₁₋₄ alkyl, C₁₋₄ haloalkyl, —OR^{a1}, —SR^{a1} and —NR^{a1}R^{b1};
R² is selected from: H, halo, C₁₋₄ alkyl and C₁₋₄ haloalkyl, —OR^{a2}, —SR^{a2} and —NR^{a2}R^{b2}
   wherein the C₁₋₄ alkyl is optionally substituted by one or more substituents selected from: halo, —OR^{a3}, —SR^{a3} and —NR^{a3}R^{b3};
R³ is selected from: C₁₋₄ alkyl, C₁₋₄ haloalkyl, C₃₋₆ cycloalkyl, C₃₋₆ cycloalkyl-C₁₋₃ alkyl-, —OR⁴, —NR⁵R⁶ and —SR⁵;
   R⁴ and R⁵ are independently selected from: H, C₁₋₄ alkyl, C₁₋₄ haloalkyl, C₃₋₆ cycloalkyl and C₃₋₆ cycloalkyl-C₁₋₃ alkyl-;
   R⁶ is selected from: H, C₁₋₄ alkyl and C₁₋₄ haloalkyl;
   wherein any C₁₋₄ alkyl, C₃₋₆ cycloalkyl or C₃₋₆ cycloalkyl-C₁₋₃ alkyl- in any of R³, R⁴, R⁵ or R⁶ is optionally substituted by one or more substituents selected from: halo, —OR^{a4}, —SR^{a4} and —NR^{a4}R^{b4};
X¹, X² and X³ are independently selected from: N and CR⁷;
   R⁷ is at each occurrence independently selected from: H, halo, —CN, C₁₋₄ alkyl, C₁₋₄ haloalkyl, —OR⁸, —NR⁸R⁹ and —S(O)ₓR⁸ (wherein x is 0, 1, or 2);
   R⁸ and R⁹ are each independently selected from: H, C₁₋₄ alkyl and C₁₋₄ haloalkyl;
   wherein any C₁₋₄ alkyl in any of R⁷, R⁸ or R⁹ is optionally substituted by one or more substituents selected from: halo, —CN, —OR^{a5}, —S(O)₂R^{a5} (wherein x is 0, 1, or 2) and —NR^{a5}R^{b5}.
Ring B is selected from C₆₋₁₀ aryl and 5- to 12-membered heteroaryl optionally substituted with one or more R¹⁰, wherein when Ring B is heteroaryl, Ring B is bonded to the remainder of the compound of Formula (I) by a ring atom in an aromatic ring of the heteroaryl;
   R¹⁰ at each occurrence is independently selected from: halo, —CN, —NO₂, ═O, C₁₋₆ alkyl, C₂₋₆ alkenyl, C₂₋₆ alkynyl, C₁₋₆ haloalkyl, Q¹-L¹-, —OR¹¹, —S(O)ₓR¹¹ (wherein x is 0, 1, or 2), —NR¹¹R^{a6}, —C(O)R¹¹, —OC(O)R¹¹, —C(O)OR¹¹, —NR^{a6}C(O)R¹¹, —NR^{a6}C(O)OR¹¹, —C(O)NR¹¹R^{a6}, —OC(O)NR¹¹R^{a6}, —NR^{a6}SO₂R¹¹, —SO₂NR¹¹R^{a6} and —NR^{a6}C(O)NR¹¹R^{a6}
   wherein said C₁₋₆ alkyl, C₂₋₆ alkenyl and C₂₋₆ alkynyl is optionally substituted by 1 or more R¹²;
   R¹¹ is independently selected from: H, C₁₋₆ alkyl and C₁₋₆ haloalkyl, wherein said C₁-6 alkyl is optionally substituted by one or more R¹³;
   Q¹ at each occurrence is independently selected from: C₃₋₆ cycloalkyl, C₃₋₆ cycloalkyl-C₁₋₃ alkyl-, 4- to 7-membered heterocyclyl, 4- to 7-membered heterocyclyl-C₁₋₃ alkyl-, phenyl, phenyl-C₁₋₃ alkyl-, 5- or 6-membered heteroaryl and 5- or 6-membered heteroaryl-C₁₋₃ alkyl-,
   wherein said C₃₋₆ cycloalkyl, C₃₋₆ cycloalkyl-C₁₋₃ alkyl-, 4- to 7-membered heterocyclyl and 4- to 7-membered heterocyclyl-C₁₋₃ alkyl- is optionally substituted by one or more R¹⁴, and wherein said phenyl, phenyl-C₁₋₃ alkyl, 5- or 6-membered heteroaryl and 5- or 6-membered heteroaryl-C₁₋₃ alkyl- is optionally substituted by one or more R¹⁵;
   L¹ is a bond or is selected from —O—, —S(O)ₓ-(wherein x is 0, 1, or 2), —NR^{a7}—, —C(O)—, —OC(O)—, —C(O)O—, —NR^{a7}C(O)—, —C(O)NR^{a7}—, —NR^{a7}C(O)O—, —OC(O)NR^{a7}—, —NR^{a7}SO₂—, —SO₂NR^{a7}— and —NR^{a7}C(O)NR^{a7}—;
   R¹², R¹³ and R¹⁴ are at each occurrence independently selected from: halo, ═O, —CN, —NO₂, C₁₋₄ alkyl, C₁₋₄ haloalkyl, —OR^{a8}, —S(O)₂R^{a8}, —NR^{a8}R^{b8}, —C(O)R^{a8}, —OC(O)R^{a8}, —C(O)OR^{a8}, —NR^{a8}C(O)R^{b8}, —C(O)NR^{a8}R^{b8}, —NR^{a8}C(O)OR^{b8}, —OC(O)NR^{a8}R^{b8}, —NR^{a8}SO₂R^{b8} and —SO₂NR^{a8}R^{b8}.
   wherein said C₁₋₄ alkyl is optionally substituted by 1 or 2 substituents selected from: halo, —CN, —OR^{a9}, —NR^{a9}R^{b9} and —SO₂R^{a9};
   R¹⁵ is at each occurrence independently selected from: halo, ═O, —CN, —NO₂, C₁₋₄ alkyl, C₁₋₄ haloalkyl, —OR^{a10}, —S(O)₂R^{a10}, —NR^{a10}R^{b10}, —C(O)R^{a10}, —OC(O)R^{a10}, —C(O)OR^{a10}, —NR^{a10}C(O)R^{b10}, —C(O)NR^{a10}R^{b10}, —NR^{a10}C(O) OR^{b10}, —OC(O)NR^{a10}R^{b10}, —NR^{b10}SO₂R^{a10} and —SO₂NR^{a10}R^{b10};
   wherein said C₁₋₄ alkyl is optionally substituted by 1 or 2 substituents selected from: halo, —CN, —OR^{a5}, —NR^{a5}R^{b5} and —SO₂R^{a5};
R^{a1}, R^{b1}, R^{a2}, R^{b2}, R^{a3}, R^{b3}, R^{a4}, R^{b4}, R^{a5}, R^{b5}, R^{a6}, R^{a7}, R^{a8}, R^{b8}, R^{a9}, R^{b9}, R^{a10} and R^{b10} are at each occurrence independently selected from: H, C₁₋₄ alkyl and C₁₋₄ haloalkyl,
   or any —NR^{a1}R^{b1}, —NR²²R^{b2}, —NR²³R^{b3}, —NR²⁴R^{b4}, —NR^{a8}R^{b8}, —NR^{a9}R^{b9}, —NR^{a10}R^{b10}, —NR⁵R⁶, —NR⁸R⁹ or —NR¹¹R^{a6} within a substituent may form a 4- to 6-membered heterocyclyl, wherein said 4- to 6-membered heterocyclyl is optionally substituted by one or more substituents selected from: halo, ═O, C₁₋₄ alkyl and C₁₋₄ haloalkyl.
P2. The compound according to clause P1, wherein R² is C₁₋₄ alkyl, for example wherein R² is methyl.
P3. The compound according to any preceding clause, wherein R¹ is:

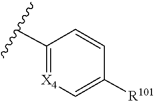

wherein:
X⁴ is CH or N; and
R¹⁰¹ is H or halo;
   optionally wherein R¹ is selected from:

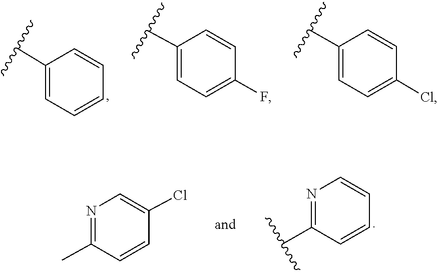

P4. The compound according to clause P1, wherein Ring A has a structure selected from:

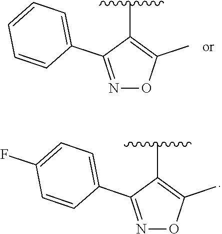 [SMILES: *c1c(-c2ccccc2)noc1C ; *c1c(-c2ccc(F)cc2)noc1C]

P5. The compound according to any preceding clause, wherein X¹ and X² are CH, optionally wherein X³ is N or CH.
P6. The compound according to any preceding clause, wherein X³ is N.
P7. The compound according to any one of clauses P1 to P4, wherein X² and X³ are N and X¹ is CR⁷, optionally wherein X¹ is CH.
P8. The compound according to any preceding clause, wherein R³ is selected from: —OR⁴ and —NR⁵R⁶; optionally wherein:
   R⁴ is selected from: C₃₋₆ cycloalkyl, C₁₋₄ alkyl and C₂₋₄ alkyl optionally substituted by —NR^{a4}R^{b4};
   R^{a4} and R^{b4} are independently selected from: H and C₁₋₄ alkyl; and
   R⁵ and R⁶ are independently selected from: H and C₁₋₄ alkyl.
P9. The compound according to any one of clauses P1 to P7, wherein:
   (i) R³ is —OR⁴, optionally wherein R⁴ is selected from: C₁₋₄ alkyl and C₃₋₆ cycloalkyl; or
   (ii) R³ is selected from: methoxy,

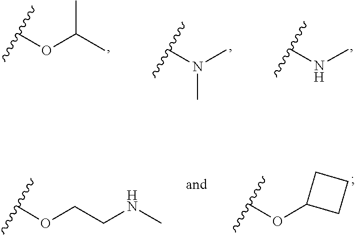

or
(iii) R³ is selected from: methoxy and

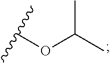 [SMILES: *OC(C)C]

or
(iv) R³ is methoxy.
P10. The compound according to any one of clauses P1 to P9, wherein:
   (i) Ring B is selected from: phenyl and a monocyclic or bicyclic 5- to 12-membered heteroaryl, each of which is optionally substituted with one or more R¹⁰; or
   (ii) Ring B is selected from: a 5- or 6-membered heteroaryl, each of which is optionally substituted with one or more R¹⁰; or
   (iii) Ring B is selected from: a bicyclic 9- or 10-membered heteroaryl optionally substituted with one or more R¹⁰;
   optionally wherein the said heteroaryl in any of (i), (ii) and (iii) contains 1 ring nitrogen atom and optionally 1 to 3 ring heteroatoms selected from O, S and N.
P11. The compound according to any one of clauses P1 to P9, wherein Ring B is selected from:

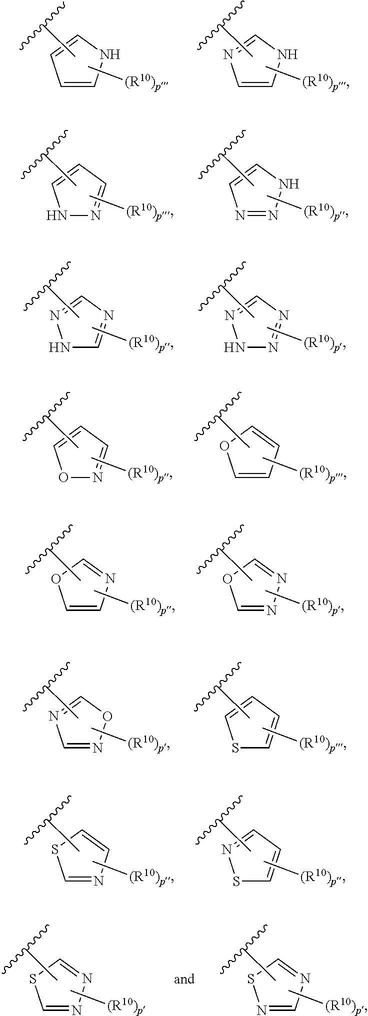

wherein p′ is 0 or 1;
p″ is 0, 1 or 2;
p′″ is 0, 1, 2 or 3; and
[symbol] indicates the point of attachment to the remainder of the compound of Formula (I).
P12. The compound according to any one of clauses P1 to P11, wherein Ring B is bonded to the remainder of the compound of Formula (I) via a ring carbon atom in an aromatic ring in Ring B.
P13. The compound according to any one of clauses P1 to P11, wherein Ring B is bonded to the remainder of the compound of Formula (I) via a ring nitrogen atom in an aromatic ring in Ring B.
P14. The compound according to any one of clauses P1 to P9, wherein:
   (i) Ring B is:

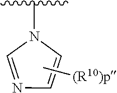

wherein p″ is 0, 1 or 2, optionally wherein p′″ is 0; or
   (ii) Ring B is selected from:

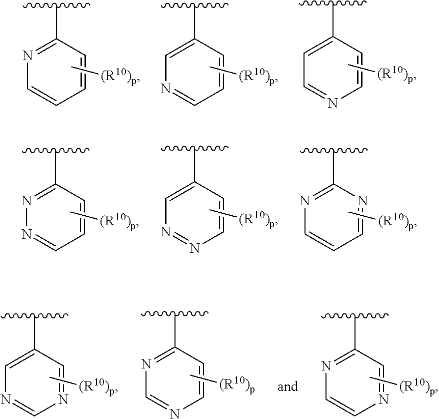

wherein p is 0, 1, 2 or 3, and wherein [symbol] indicates the point of attachment to the remainder of the compound of Formula (I); or
(iii) Ring B is selected from:

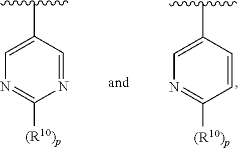

wherein p is 0 or 1; and [symbol] indicates the point of attachment to the remainder of the compound of Formula (I); optionally wherein p is 1.
P15. The compound according to any preceding clause, wherein:
   (i) R¹⁰ at each occurrence is independently selected from: halo, —CN, C₁₋₄ alkyl, C₁-4 haloalkyl, —C₁₋₄ alkyl-NR^{a8}R^{b8}, —OH, —OC₁₋₄ alkyl, —OC₁₋₄ haloalkyl, —OC₂₋₄ alkyl-NR^{a8}R^{b8}, —NH₂, —NR^{a6}C₁₋₄ alkyl, —NR^{a6}C₂₋₄ alkyl-OR^{a8}, —NR^{a6}C₂₋₄ alkyl-NR^{a8}R^{b8}, —C(O)C₁₋₄ alkyl, —C(O)C₁₋₄ haloalkyl, —C(O)C₁₋₄ alkyl-NR^{a8}R^{b8}, —COOH, —C(O)OC₁₋₄ alkyl, —C(O)NR^{a6}C₁₋₄ alkyl, —C(O)NR^{a6}C₂₋₄ alkyl-OR^{a8}, —C(O)NR^{a6}C₂₋₄ alkyl-NR^{a8}R^{b8} and Q¹⁰²-L¹⁰²-;
   wherein Q¹⁰² is selected from 4 to 6 membered heterocyclyl and 5- or 6-membered heteroaryl,
   wherein said 4 to 6 membered heterocyclyl is selected from: azetidinyl, pyrrolidinyl, piperidinyl, piperazinyl and morpholinyl, each of which is optionally substituted by one or more (e.g. 1 or 2) substituents selected from: halo, ═O, C₁₋₄ alkyl, —OR^{a8}, —NR^{a8}R^{b8} and —C(O)R^{a8},
   wherein said 5- or 6-membered heteroaryl is selected from: pyrrolyl, imidazolyl, oxazolyl, isoxazolyl, thiazolyl, isothiazolyl, pyrazolyl, oxadiazolyl, triazolyl, pyridyl, pyrimidyl, pyrazinyl and pyridazinyl, each of which is optionally substituted by one or more (e.g. 1 or 2) substituents selected from: halo, C₁₋₄ alkyl, —OR^{a10} and —NR^{a10}R^{b10};
L¹⁰² is a bond or is selected from: C₁₋₃ alkylene, —O— and —NR^{a7}—; or
   (ii) R¹⁰ is selected from: —NR^{a81}R^{b81}, —C₁₋₃ alkyl-NR^{a81}R^{b81}, —NR^{a81}—C₂₋₃ alkyl-NR^{a81}R^{b81}, —C(O)C₁₋₃ alkyl-NR^{a81}R^{b81}, and —C(O)NR^{a81}C₂₋₃ alkyl-NR^{a81}R^{b81}, wherein R^{a81} and R^{b81} are independently selected from H and C₁₋₃ alkyl; or
   (iii) R¹⁰ is selected from: —NR^{a81}R^{b81} and —C₁₋₃ alkyl-NR^{a81}R^{b81}, wherein R^{a81} and R^{b81} are independently selected from H and C₁₋₃ alkyl; or
   (iv) R¹⁰ is selected from: fluoro, chloro, cyano, nitro, oxo, hydroxy, methyl, ethyl, isopropyl, cyclopropyl, amino —NH(Me), —N(Me)₂,

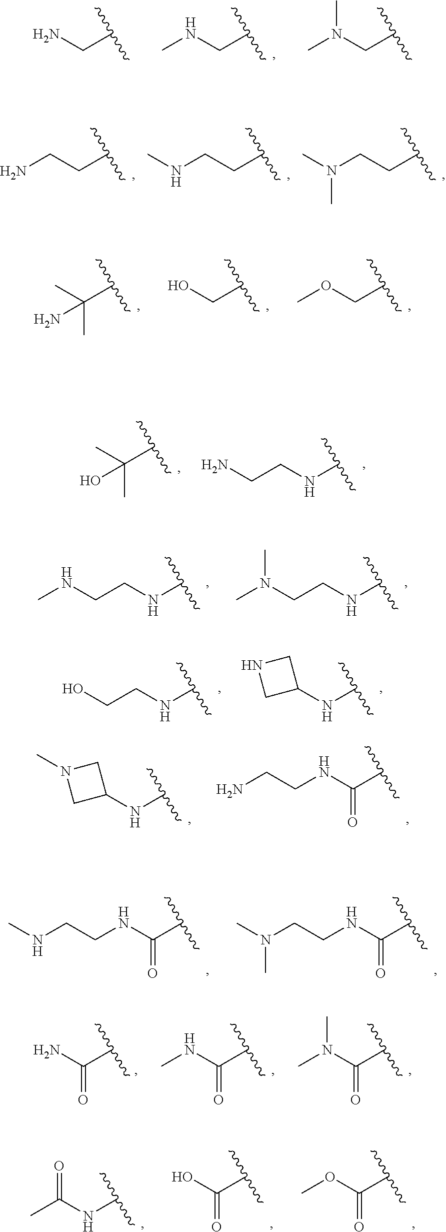

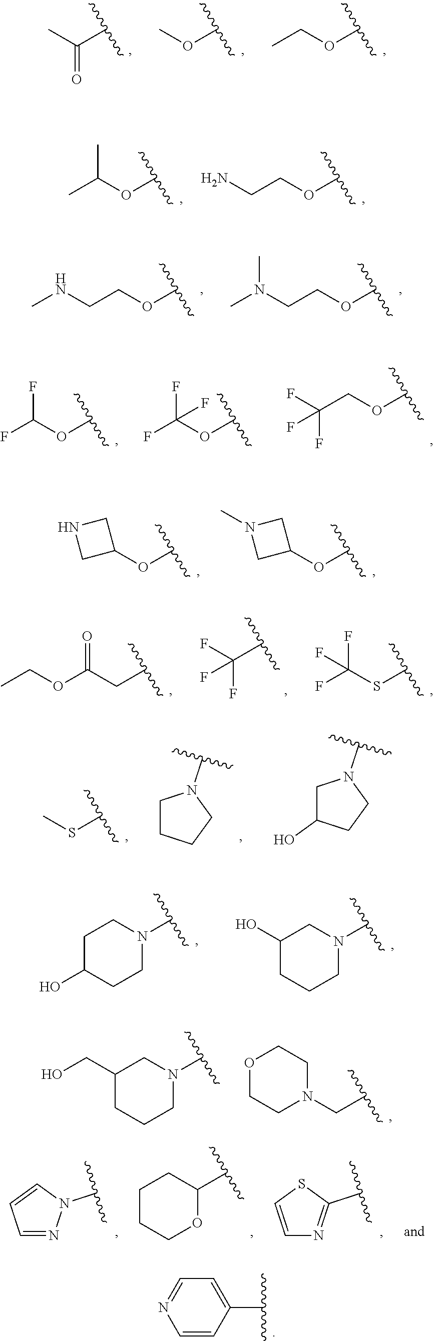

P16. The compound according to any one of clauses P1 to P9, wherein Ring B is selected from:

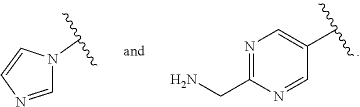 [SMILES: *n1ccnc1 ; *c1cnc(CN)nc1]

P17. The compound according to clause 1, wherein the compound is selected from Compound List 1 in the description, or a pharmaceutically acceptable salt thereof.
P18. A pharmaceutical composition comprising a compound of any one of clauses P1 to P17, or a pharmaceutically acceptable salt thereof, and a pharmaceutically acceptable excipient.
P19. A compound of any one of clauses P1 to P17, or a pharmaceutically acceptable salt thereof, or a pharmaceutical composition of clause P8, for use as a medicament.
P20. A compound of any one of clauses P1 to P17, or a pharmaceutically acceptable salt thereof, or a pharmaceutical composition of clause P18, for use in the prevention or treatment of a disease or medical disorder mediated by α5-GABA_{A} receptors.
P22. A compound of any one of clauses P1 to P17, or a pharmaceutically acceptable salt thereof, or a pharmaceutical composition of clause P18, for use in the prevention or treatment of cognitive dysfunction associated with a disease or medical disorder mediated by α5-GABA_{A} receptors.
P22. A method of preventing or treating a disease or medical disorder mediated by α5-GABA_{A}Rs in a subject, the method comprising administering to the subject an effective amount of a compound of any one of clauses P1 to P17, or a pharmaceutically acceptable salt thereof, or a pharmaceutical composition of clause P18.
P23. The compound for the use of clause P20 or clause P21, or the method of clause P22, wherein disease or medical disorder mediated by α5-GABA_{A}Rs is selected from: Alzheimer's disease, Parkinson's disease, Huntington's disease, cognitive dysfunction (e.g. cognitive dysfunction associated with chemotherapy, an anaesthetic, a bacterial infection or a viral infection (e.g. HIV), memory deficit, age-related cognitive impairment (e.g. mild cognitive impairment, MCI), a bipolar disorder, autism, Down syndrome, neurofibromatosis type I, a sleep disorder, a disorder of circadian rhythms, amyotrophic lateral sclerosis (ALS), a psychotic disorder (e.g. schizophrenia, schizoaffective disorder, schizophreniform disorder, substance-induced psychotic disorder or paraphrenia), psychosis, post-traumatic stress disorder, an anxiety disorder, a generalized anxiety disorder, a panic disorder, a delusional disorder, an obsessive/compulsive disorder, an acute stress disorder, drug addiction, alcohol disorders (e.g. alcohol addiction), drug withdrawal symptoms, a movement disorder, restless leg syndrome, a cognition deficiency disorder, multi-infarct dementia, vascular dementia, a mood disorder, depression, a neuropsychiatric condition, attention-deficit/hyperactivity disorder, neuropathic pain, chronic neuroinflammation, cognitive dysfunction associated with stroke, cognitive dysfunction associated with brain injury or trauma, cognitive dysfunction associated with a brain tumour and an attentional disorder;
   optionally the compound of any one of clauses P1 to P17, or a pharmaceutically acceptable salt thereof, or the pharmaceutical composition of clause P18 for use in the treatment or prevention of post-operative cognitive dysfunction, for example anaesthetic induced cognitive dysfunction.
P24. A compound of any one of clauses P1 to 17, or a pharmaceutically acceptable salt thereof, or a pharmaceutical composition of clause P18, for use in the prevention or treatment of a neurological or neuropsychiatric disorder mediated by α5-GABA_{A}Rs (e.g. the treatment or prevention of cognitive dysfunction associated with a neurological disorder or neuropsychiatric disorder);
   optionally wherein the neurological disorder is a neurodevelopment disorder (e.g. attention deficit disorder (ADHD), Down Syndrome, a learning disability, cerebral palsy, autism or a speech disorder);
   optionally wherein the neurodegenerative condition (e.g. Alzheimer's disease, dementia, Parkinson's disease, Huntington's disease, amyotrophic lateral sclerosis (ALS) or Creutzfeldt-Jakob disease (CJD));
   for example wherein the neurological disorder is Huntington's disease.
P25. A compound of any one of clauses P1 to P17, or a pharmaceutically acceptable salt thereof, or a pharmaceutical composition of clause P18, for use in the for use in the treatment or prevention of psychiatric and/or neurological symptoms (particularly cognitive dysfunction) caused by or associated with a viral or bacterial infection.
1. A compound of the formula (I), or a pharmaceutically acceptable salt thereof:

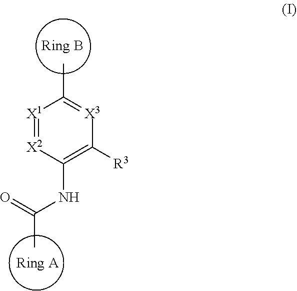

wherein
Ring A is selected from: A1, A2 and A3:

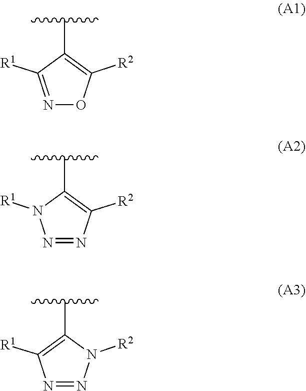

R¹ is selected from: phenyl and a 5- or 6-membered heteroaryl, wherein R¹ is optionally substituted by one or more substituents selected from: halo, C₁₋₄ alkyl, C₁₋₄ haloalkyl, —OR^{a1}, —SR^{a1} and —NR^{a1}R^{b1};
R² is selected from: H, halo, C₁₋₄ alkyl and C₁₋₄ haloalkyl, —OR^{a2}, —SR^{a2} and —NR^{a2}R^{b2}
   wherein the C₁₋₄ alkyl is optionally substituted by one or more substituents selected from: halo, —OR^{a3}, —SR^{a3} and —NR^{a3}R^{b3};
R³ is selected from: C₁₋₄ alkyl, C₁₋₄ haloalkyl, C₃₋₆ cycloalkyl, C₃₋₆ cycloalkyl-C₁₋₃ alkyl-, —OR⁴, —NR⁵R⁶, —SR⁵, 4- to 7-membered heterocyclyl containing 1 or more ring oxygen atoms, and 4- to 7-membered heterocyclyl-C₁₋₃ alkyl-containing 1 or more ring oxygen atoms;
   R⁴ and R⁵ are independently selected from: H, C₁₋₄ alkyl, C₁₋₄ haloalkyl, C₃₋₆ cycloalkyl, C₃₋₆ cycloalkyl-C₁₋₃ alkyl-, 4- to 7-membered heterocyclyl containing 1 or more ring oxygen atoms, and 4- to 7-membered heterocyclyl-C₁₋₃ alkyl-containing 1 or more ring oxygen atoms;
   R⁶ is selected from: H, C₁₋₄ alkyl and C₁₋₄ haloalkyl;
   wherein any C₁₋₄ alkyl, C₃₋₆ cycloalkyl or C₃₋₆ cycloalkyl-C₁₋₃ alkyl- in any of R³, R⁴, R⁵ or Re is optionally substituted by one or more substituents selected from: halo, —OR^{a4}, —SR^{a4} and —NR^{a4}R^{b4},
X¹, X² and X³ are independently selected from: N and CR⁷;
   R⁷ is at each occurrence independently selected from: H, halo, —CN, C₁₋₄ alkyl, C₁₋₄ haloalkyl, —OR⁸, —NR⁸R⁹ and —S(O)ₓR⁸ (wherein x is 0, 1, or 2);
   R⁸ and R⁹ are each independently selected from: H, C₁₋₄ alkyl and C₁₋₄ haloalkyl;
   wherein any C₁₋₄ alkyl in any of R⁷, R⁸ or R⁹ is optionally substituted by one or more substituents selected from: halo, —CN, —OR^{a5}, —S(O)ₓR^{a5} (wherein x is 0, 1, or 2) and —NR^{a5}R^{b5}.
Ring B is selected from C₆₋₁₀ aryl and 5- to 12-membered heteroaryl optionally substituted with one or more R¹⁰, wherein when Ring B is heteroaryl, Ring B is bonded to the remainder of the compound of Formula (I) by a ring atom in an aromatic ring of the heteroaryl;
   R¹⁰ at each occurrence is independently selected from: halo, —CN, —NO₂, ═O, C₁₋₆ alkyl, C₂₋₆ alkenyl, C₂₋₆ alkynyl, C₁₋₆ haloalkyl, Q¹-L¹-, —OR¹¹, —S(O)ₓR¹¹ (wherein x is 0, 1, or 2), —NR¹¹R^{a6}, —C(O)R¹¹, —OC(O)R¹¹, —C(O)OR¹¹, —NR^{a6}C(O)R¹¹, —NR^{a6}C(O)OR¹¹, —C(O)NR¹¹R^{a6}, —OC(O)NR¹¹R^{a6}, —NR^{a6}SO₂R¹¹, —SO₂NR¹¹R^{a6} and —NR^{a6}C(O)NR¹¹R^{a6},
   wherein said C₁₋₆ alkyl, C₂₋₆ alkenyl and C₂₋₆ alkynyl is optionally substituted by 1 or more R¹².
   R¹¹ is independently selected from: H, C₁₋₆ alkyl and C₁₋₆ haloalkyl, wherein said C₁₋₆ alkyl is optionally substituted by one or more R¹³;
   Q¹ at each occurrence is independently selected from: C₃₋₆ cycloalkyl, C₃₋₆ cycloalkyl-C₁₋₃ alkyl-, 4- to 7-membered heterocyclyl, 4- to 9-membered heterocyclyl-C₁₋₃ alkyl-, phenyl, phenyl-C₁₋₃ alkyl-, 5- or 6-membered heteroaryl and 5- or 6-membered heteroaryl-C₁₋₃ alkyl-,
   wherein said C₃₋₆ cycloalkyl, C₃₋₆ cycloalkyl-C₁₋₃ alkyl-, 4- to 7-membered heterocyclyl and 4- to 7-membered heterocyclyl-C₁₋₃ alkyl- is optionally substituted by one or more R¹⁴, and
   wherein said phenyl, phenyl-C₁₋₃ alkyl-, 5- or 6-membered heteroaryl and 5- or 6-membered heteroaryl-C₁₋₃ alkyl- is optionally substituted by one or more R¹⁵;
   L¹ is a bond or is selected from —O—, —S(O)ₓ-(wherein x is 0, 1, or 2), —NR^{a7}—, —C(O)—, —OC(O)—, —C(O)O—, —NR^{a7}C(O)—, —C(O)NR^{a7}—, —NR^{a7}C(O)O—, —OC(O)NR^{a7}—, —NR^{a7}SO₂—, —SO₂NR^{a7}— and —NR^{a7}C(O)NR^{a7}—;
   R¹², R¹³ and R¹⁴ are at each occurrence independently selected from: halo, ═O, —CN, —NO₂, C₁₋₄ alkyl, C₁₋₄ haloalkyl, —OR^{a8}, —S(O)₂R^{a8}, —NR^{a8}R^{b8}, —C(O)R^{a8}, —OC(O)R^{a8}, —C(O)OR^{a8}, —NR^{a8}C(O)R^{b8}, —C(O)NR^{a8}R^{b8}, —NR^{a8}C(O)OR^{b8}, —OC(O)NR^{a8}R^{b8}, —NR^{a8}SO₂R^{b8} and —SO₂NR^{a8}R^{b8}.
   wherein said C₁₋₄ alkyl is optionally substituted by 1 or 2 substituents selected from: halo, —CN, —OR^{a9}, —NR^{a9}R^{b9} and —SO₂R^{a9};
   R¹⁵ is at each occurrence independently selected from: halo, ═O, —CN, —NO₂, C₁₋₄ alkyl, C₁₋₄ haloalkyl, —OR^{a10}, —S(O)₂R^{a10}, —NR^{a10}R^{b10}, —C(O)R^{a10}, —OC(O)R^{a10}, —C(O)OR^{a10}, —NR^{a10}C(O)R^{b10}, —C(O)NR^{a10}R^{b10}, —NR^{a10}C(O) OR^{b10}, —OC(O)NR^{a10}R^{b10}, —NR^{b10}SO₂R^{a10} and —SO₂NR^{a10}R^{b10};
   wherein said C₁₋₄ alkyl is optionally substituted by 1 or 2 substituents selected from: halo, —CN, —OR^{a5}, —NR^{a11}R^{b11} and —SO₂R^{a11};
R^{a1}, R^{b1}, R^{a2}, R^{b2}, R^{a3}, R^{b3}, R^{a4}, R^{b4}, R^{a5}, R^{b5}, R^{a6}, R^{a7}, R^{a8}, R^{b8}, R^{a9}, R^{b9}, R^{a10}, R^{b10}, R^{a11} and R^{b11} are at each occurrence independently selected from: H, C₁₋₄ alkyl and C₁₋₄ haloalkyl,
   or any —NR^{a1}R^{b1}, —NR^{a2}R^{b2}, —NR^{a3}R^{b3}, —NR^{a4}R^{b4}, —NR^{a5}R^{b5}, —NR^{a8}R^{b8}, —NR^{a9}R^{b9}, —NR^{a10}R^{b10}, —NR^{a11}R^{b11}, —NR⁵R⁶, —NR⁸R⁹ or —NR¹¹R^{a6} within a substituent may form a 4- to 6-membered heterocyclyl, wherein said 4- to 6-membered heterocyclyl is optionally substituted by one or more substituents selected from: halo, ═O, C₁₋₄ alkyl and C₁₋₄ haloalkyl,
   with the provisos (i) and (ii):
   (i) when Ring A is A2, and R² is H, then R³ is not —NR⁵R⁶; and
   (ii) the compound of formula (I) is not

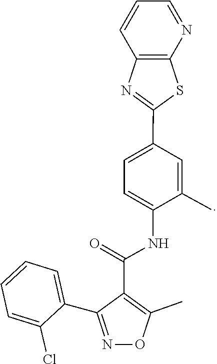 [SMILES: Cc1cc(-c2nc3cccnc3s2)ccc1NC(=O)c1c(-c2ccccc2Cl)noc1C]

2. The compound according to clause 1, wherein R² is C₁₋₄ alkyl, for example wherein R² is methyl.
3. The compound according to any preceding clause, wherein R¹ is:

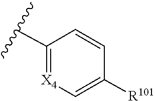

wherein:
X⁴ is CH or N; and
R¹⁰¹ is H or halo;
   optionally wherein R¹ is selected from:

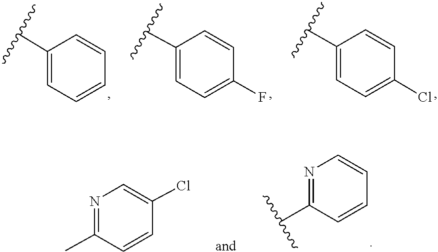

4. The compound according to clause 1, wherein Ring A has a structure selected from:

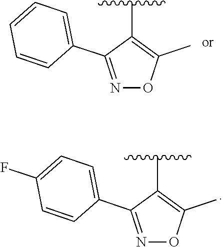 [SMILES: *c1c(-c2ccccc2)noc1C ; *c1c(-c2ccc(F)cc2)noc1C]

5. The compound according to any preceding clause, wherein X¹ and X² are CH, optionally wherein X³ is N or CH.
6. The compound according to any preceding clause, wherein X³ is N.
7. The compound according to any one of clauses 1 to 4, wherein X² and X³ are N and X¹ is CR⁷, optionally wherein X¹ is CH.
8. The compound according to any preceding clause, wherein R³ is selected from: —OR⁴ and —NR⁵R⁶; optionally wherein:
   R⁴ is selected from: C₃₋₆ cycloalkyl, C₁₋₄ alkyl and C₂₋₄ alkyl optionally substituted by —NR^{a4}R^{b4}.
   R^{a4} and R^{b4} are independently selected from: H and C₁₋₄ alkyl; and
   R⁵ and R⁶ are independently selected from: H and C₁₋₄ alkyl.
9. The compound according to any one of clauses 1 to 7, wherein R³ is —OR⁴, optionally wherein R⁴ is selected from: C₁₋₄ alkyl and C₃₋₆ cycloalkyl.
10. The compound according to any one of clauses 1 to 7, wherein:
   (i) R³ is selected from: methoxy,

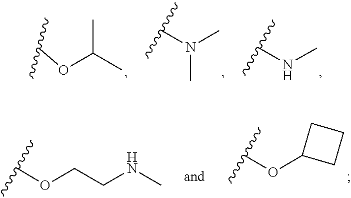

or
(ii) R³ is selected from: methoxy,

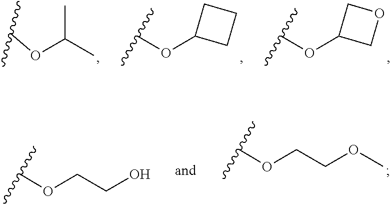

or
(iii) R³ is —OR⁴, wherein R⁴ is 4- to 6-membered heterocyclyl containing 1 ring oxygen atom, or 4- to 6-membered heterocyclyl-C₁₋₃ alkyl-containing 1 ring oxygen atom, for example R³ is:

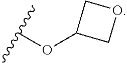 [SMILES: *OC1COC1]

11. The compound according to any one of clauses 1 to 7, wherein R³ is selected from: methoxy and

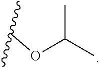 [SMILES: *OC(C)C]

12. The compound according to any one of clauses 1 to 7, wherein R³ is methoxy.
13. The compound according to any one of clauses 1 to 12, wherein Ring B is selected from: phenyl and a monocyclic or bicyclic 5- to 12-membered heteroaryl, each of which is optionally substituted with one or more R¹⁰.
14. The compound according to any one of clauses 1 to 12, wherein Ring B is selected from: a 5- or 6-membered heteroaryl, each of which is optionally substituted with one or more R¹⁰.
15. The compound according to any one of clauses 1 to 12, wherein Ring B is selected from: a bicyclic 9- or 10-membered heteroaryl optionally substituted with one or more R¹⁰.
16. The compound according to any one of clauses 13 to 15, wherein the heteroaryl contains 1 ring nitrogen atom and optionally 1 to 3 ring heteroatoms selected from O, S and N.
17. The compound according to any one of clauses 1 to 12, wherein Ring B is selected from:

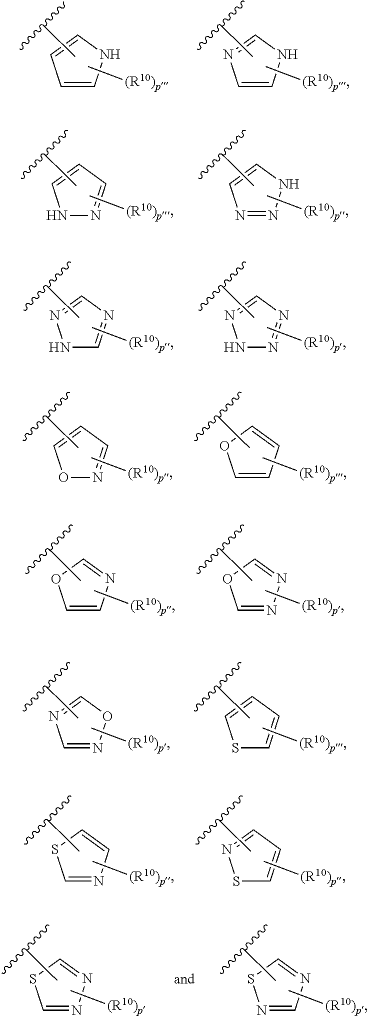

wherein p′ is 0 or 1;
p″ is 0, 1 or 2;
p′″ is 0, 1, 2 or 3; and
[symbol] indicates the point of attachment to the remainder of the compound of Formula (I).
18. The compound according to any one of clauses 1 to 17, wherein Ring B is bonded to the remainder of the compound of Formula (I) via a ring carbon atom in an aromatic ring in Ring B.
19. The compound according to any one of clauses 1 to 17, wherein Ring B is bonded to the remainder of the compound of Formula (I) via a ring nitrogen atom in an aromatic ring in Ring B.
20. The compound according to any one of clauses 1 to 12, wherein Ring B is:

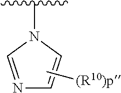

wherein p″ is 0, 1 or 2, optionally wherein p′″ is 0.
21. The compound according to any one of clauses 1 to 12, wherein Ring B is selected from:

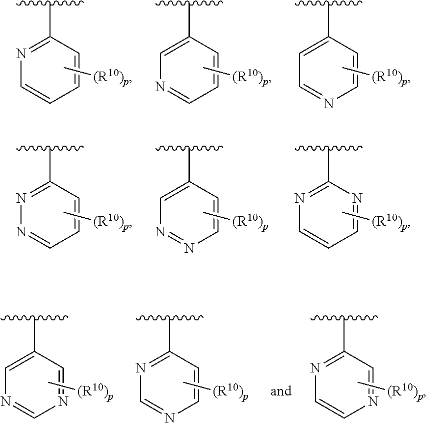

wherein p is 0, 1, 2 or 3, and wherein [symbol] indicates the point of attachment to the remainder of the compound of Formula (I).
22. The compound according to any one of clauses 1 to 12, wherein Ring B is selected from:

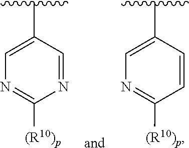

wherein p is 0 or 1; and [symbol] indicates the point of attachment to the remainder of the compound of Formula (I); optionally wherein p is 1.
23. The compound according to any preceding clause, wherein R¹⁰ at each occurrence is independently selected from: halo, —CN, C₁₋₄ alkyl, C₁₋₄ haloalkyl, —C₁₋₄ alkyl-NR^{a8}R^{b8}, —OH, —OC₁₋₄ alkyl, —OC₁₋₄ haloalkyl, —OC₂₋₄ alkyl-NR^{a8}R^{b8}, —NH₂, —NR^{a6}C₁₋₄ alkyl, —NR^{a6}C₂₋₄ alkyl-OR^{a8}, —NR^{a6}C₂₋₄ alkyl-NR^{a8}R^{b8}, —C(O)C₁₋₄ alkyl, —C(O)C₁₋₄ haloalkyl, —C(O)C₁₋₄ alkyl-NR^{a8}R^{b8}, —COOH, —C(O)OC₁₋₄ alkyl, —C(O)NR^{a6}C₁₋₄ alkyl, —C(O)NR^{a6}C₂₋₄ alkyl-OR^{a8}, —C(O)NR^{a6}C₂₋₄ alkyl-NR^{a8}R^{b8} and Q¹⁰²-L¹⁰²-;
   wherein Q¹⁰² is selected from 4 to 6 membered heterocyclyl and 5- or 6-membered heteroaryl,
   wherein said 4 to 6 membered heterocyclyl is selected from: azetidinyl, pyrrolidinyl, piperidinyl, piperazinyl and morpholinyl, each of which is optionally substituted by one or more (e.g. 1 or 2) substituents selected from: halo, ═O, C₁₋₄ alkyl, —OR^{a8}, —NR^{a8}R^{b8} and —C(O)R^{a8},
   wherein said 5- or 6-membered heteroaryl is selected from: pyrrolyl, imidazolyl, oxazolyl, isoxazolyl, thiazolyl, isothiazolyl, pyrazolyl, oxadiazolyl, triazolyl, pyridyl, pyrimidyl, pyrazinyl and pyridazinyl, each of which is optionally substituted by one or more (e.g. 1 or 2) substituents selected from: halo, C₁₋₄ alkyl, —OR^{a10} and —NR^{a10}R^{b10};
L¹⁰² is a bond or is selected from: C₁₋₃ alkylene, —O— and —NR^{a7}—.
24. The compound according to any one of clauses 1 to 22, wherein R¹⁰ is selected from: —NR^{a81}R^{b81}, —C₁₋₃ alkyl-NR^{a81}R^{b81}, —NR^{a81}—C₂₋₃ alkyl-NR^{a81}R^{b81}, —C(O)C₁₋₃ alkyl-NR^{a81}R^{b81}, and —C(O)NR^{a81}C₂₋₃ alkyl-NR^{a81}R^{b81}, wherein R^{a81} and R^{b81} are independently selected from H and C₁₋₃ alkyl.
25. The compound according to any one of clauses 1 to 22, wherein R¹⁰ is selected from: —NR^{a81}R^{b81} and —C₁₋₃ alkyl-NR^{a81}R^{b81}, wherein R^{a81} and R^{b81} are independently selected from H and C₁₋₃ alkyl.
26. The compound according to any one of clauses 1 to 22, wherein R¹⁰ is selected from: fluoro, chloro, cyano, nitro, oxo, hydroxy, methyl, ethyl, isopropyl, cyclopropyl, amino —NH(Me), —N(Me)₂,

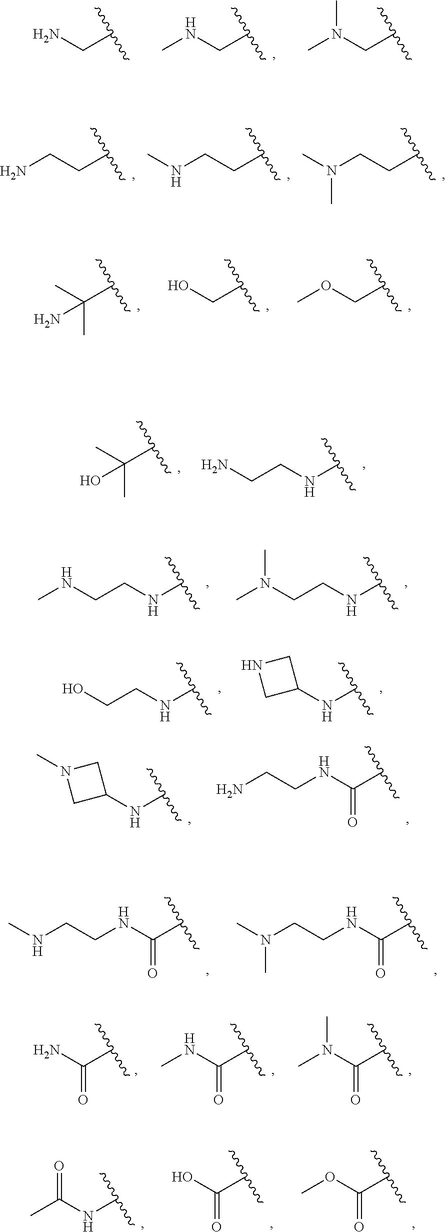

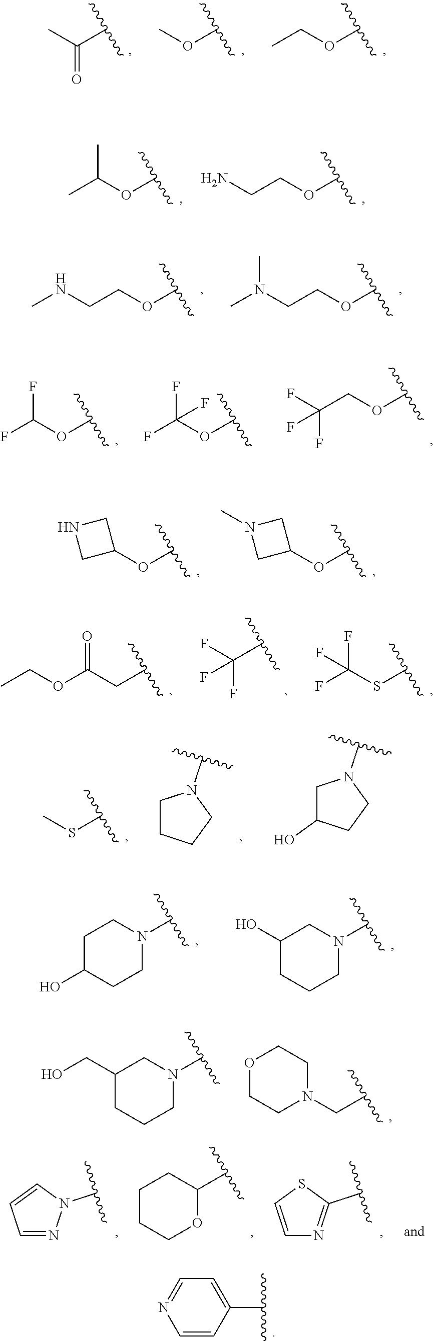

27. The compound according to any one of clauses 1 to 12, wherein Ring B is selected from:

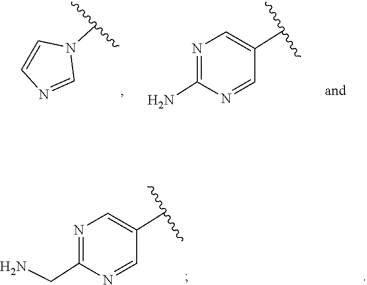 [SMILES: *n1ccnc1 ; *c1cnc(N)nc1 ; *c1cnc(CN)nc1]

optionally wherein Ring B is

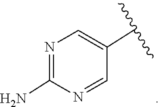 [SMILES: *c1cnc(N)nc1]

28. The compound according to clause 1, wherein the compound is selected from Compound List 1 in the description, or a pharmaceutically acceptable salt thereof.
29. A pharmaceutical composition comprising a compound of any one of clauses 1 to 28, or a pharmaceutically acceptable salt thereof, and a pharmaceutically acceptable excipient.
30. A compound of any one of clauses 1 to 28, or a pharmaceutically acceptable salt thereof, or a pharmaceutical composition of clause 29, for use as a medicament.
31. A compound of any one of clauses 1 to 28, or a pharmaceutically acceptable salt thereof, or a pharmaceutical composition of clause 29, for use in the prevention or treatment of a disease or medical disorder mediated by α5-GABA_{A} receptors.
32. A compound of any one of clauses 1 to 28, or a pharmaceutically acceptable salt thereof, or a pharmaceutical composition of clause 29, for use in the prevention or treatment of cognitive dysfunction associated with a disease or medical disorder mediated by α5-GABA_{A} receptors.
33. A method of preventing or treating a disease or medical disorder mediated by α5-GABA_{A}Rs in a subject, the method comprising administering to the subject an effective amount of a compound of any one of clauses 1 to 28, or a pharmaceutically acceptable salt thereof, or a pharmaceutical composition of clause 29.
34. The compound for the use of clause 31 or clause 32, or the method of clause 33, wherein disease or medical disorder mediated by α5-GABA_{A}Rs is selected from: Alzheimer's disease, Parkinson's disease, Huntington's disease, cognitive dysfunction (e.g. cognitive dysfunction associated with chemotherapy, an anaesthetic, a bacterial infection or a viral infection (e.g. HIV), memory deficit, age-related cognitive impairment (e.g. mild cognitive impairment, MCI), a bipolar disorder, autism, Down syndrome, neurofibromatosis type I, a sleep disorder, a disorder of circadian rhythms, amyotrophic lateral sclerosis (ALS), a psychotic disorder (e.g. schizophrenia, schizoaffective disorder, schizophreniform disorder, substance-induced psychotic disorder or paraphrenia), psychosis, post-traumatic stress disorder, an anxiety disorder, a generalized anxiety disorder, a panic disorder, a delusional disorder, an obsessive/compulsive disorder, an acute stress disorder, drug addiction, alcohol disorders (e.g. alcohol addiction), drug withdrawal symptoms, a movement disorder, restless leg syndrome, a cognition deficiency disorder, multi-infarct dementia, vascular dementia, a mood disorder, depression, a neuropsychiatric condition, attention-deficit/hyperactivity disorder, neuropathic pain, chronic neuroinflammation, cognitive dysfunction associated with stroke, cognitive dysfunction associated with brain injury or trauma, cognitive dysfunction associated with a brain tumour and an attentional disorder;
   optionally the compound of any one of clauses 1 to 28, or a pharmaceutically acceptable salt thereof, or the pharmaceutical composition of clause 29 for use in the treatment or prevention of post-operative cognitive dysfunction, for example anaesthetic induced cognitive dysfunction.
35. A compound of any one of clauses 1 to 28, or a pharmaceutically acceptable salt thereof, or a pharmaceutical composition of clause 29, for use in the prevention or treatment of a neurological or neuropsychiatric disorder mediated by α5-GABA_{A}Rs (e.g. the treatment or prevention of cognitive dysfunction associated with a neurological disorder or neuropsychiatric disorder;
   optionally wherein the neurological disorder is a neurodevelopment disorder (e.g. attention deficit disorder (ADHD), Down Syndrome, a learning disability, cerebral palsy, autism or a speech disorder);
   optionally wherein the neurodegenerative condition (e.g. Alzheimer's disease, dementia, Parkinson's disease, Huntington's disease, amyotrophic lateral sclerosis (ALS) or Creutzfeldt-Jakob disease (CJD));
for example wherein the neurological disorder is Huntington's disease.
36. A compound of any one of clauses 1 to 28, or a pharmaceutically acceptable salt thereof, or a pharmaceutical composition of clause 29, for use in the treatment or prevention of psychiatric and/or neurological symptoms (particularly cognitive dysfunction) caused by or associated with a viral or bacterial infection.

1. A compound of Formula (I), or a pharmaceutically acceptable salt thereof: 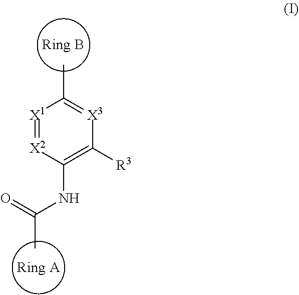
wherein
Ring A is selected from: A1, A2 and A3: 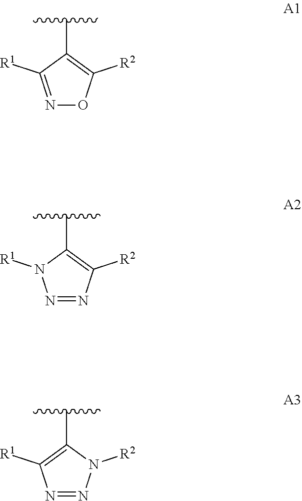
R¹ is selected from: phenyl and a 5- or 6-membered heteroaryl, wherein R¹ is optionally substituted by one or more substituents selected from: halo, C₁₋₄ alkyl, C₁₋₄ haloalkyl, —OR^{a1}, —SR^{a1} and —NR^{a1}R^{b1};
R² is selected from: H, halo, C₁₋₄ alkyl, C₁₋₄ haloalkyl, —OR^{a2}, —SR^{a2} and —NR^{a2}R^{b2},
wherein the C₁₋₄ alkyl is optionally substituted by one or more substituents selected from: halo, —OR^{a3}, —SR^{a3} and —NR^{a3}R^{b3};
R³ is selected from: C₁₋₄ alkyl, C₁₋₄ haloalkyl, C₃₋₆ cycloalkyl, C₃₋₆ cycloalkyl-C₁₋₃ alkyl-, —OR⁴, —NR⁵R⁶, —SR⁵, 4- to 7-membered heterocyclyl containing 1 or more ring oxygen atoms, and 4- to 7-membered heterocyclyl-C₁₋₃ alkyl-containing 1 or more ring oxygen atoms;
R⁴ and R⁵ are independently selected from: H, C₁₋₄ alkyl, C₁₋₄ haloalkyl, C₃₋₆ cycloalkyl, C₃₋₆ cycloalkyl-C₁₋₃ alkyl-, 4- to 7-membered heterocyclyl containing 1 or more ring oxygen atoms, and 4- to 7-membered heterocyclyl-C₁₋₃ alkyl-containing 1 or more ring oxygen atoms;
R⁶ is selected from: H, C₁₋₄ alkyl and C₁₋₄ haloalkyl;
wherein any C₁₋₄ alkyl, C₃₋₆ cycloalkyl or C₃₋₆ cycloalkyl-C₁₋₃ alkyl- in any of R³, R⁴, R⁵ or R⁶ is optionally substituted by one or more substituents selected from: halo, —OR^{a4}, —SR^{a4} and —NR^{a4}R^{b4};
X¹, X² and X³ are independently selected from: N and CR⁷;
R⁷ is at each occurrence independently selected from: H, halo, —CN, C₁₋₄ alkyl, C₁₋₄ haloalkyl, —OR⁸, —NR⁸R⁹ and —S(O)ₓR⁸ (wherein x is 0, 1, or 2);
R⁸ and R⁹ are each independently selected from: H, C₁₋₄ alkyl and C₁₋₄ haloalkyl;
wherein any C₁₋₄ alkyl in any of R⁷, R⁸ or R⁹ is optionally substituted by one or more substituents selected from: halo, —CN, —OR^{a5}, —S(O)ₓR^{a5} (wherein x is 0, 1, or 2) and —NR^{a5}R^{b5};
Ring B is selected from C₆₋₁₀ aryl and 5- to 12-membered heteroaryl optionally substituted with one or more R¹⁰, wherein when Ring B is heteroaryl, Ring B is bonded to the remainder of the compound of Formula (I) by a ring atom in an aromatic ring of the heteroaryl;
R¹⁰ at each occurrence is independently selected from: halo, —CN, —NO₂, ═O, C₁₋₆ alkyl, C₂₋₆ alkenyl, C₂₋₆ alkynyl, C₁₋₆ haloalkyl, Q¹-L¹-, —OR¹¹, —S(O)ₓR¹¹ (wherein x is 0, 1, or 2), —NR¹¹R^{a6}, —C(O)R¹¹, —OC(O)R¹¹, —C(O)OR¹¹, —NR^{a6}C(O)R¹¹, —NR^{a6}C(O)OR¹¹, —C(O)NR¹¹R^{a6}, —OC(O)NR¹¹R^{a6}, —NR^{a6}SO₂R¹¹, —SO₂NR 11R^{a6} and —NR^{a6}C(O)NR 11R^{a6},
wherein said C₁₋₆ alkyl, C₂₋₆ alkenyl and C₂₋₆ alkynyl is optionally substituted by 1 or more R¹²;
R¹¹ is independently selected from: H, C₁₋₆ alkyl and C₁₋₆ haloalkyl, wherein said C₁₋₆ alkyl is optionally substituted by one or more R¹³;
Q¹ at each occurrence is independently selected from: C₃₋₆ cycloalkyl, C₃₋₆ cycloalkyl-C₁₋₃ alkyl-, 4- to 7-membered heterocyclyl, 4- to 9-membered heterocyclyl-C₁₋₃ alkyl-, phenyl, phenyl-C₁₋₃ alkyl-, 5- or 6-membered heteroaryl and 5- or 6-membered heteroaryl-C₁₋₃ alkyl-,
wherein said C₃₋₆ cycloalkyl, C₃₋₆ cycloalkyl-C₁₋₃ alkyl-, 4- to 7-membered heterocyclyl and 4- to 9-membered heterocyclyl-C₁₋₃ alkyl- is optionally substituted by one or more R¹⁴, and
wherein said phenyl, phenyl-C₁₋₃ alkyl-, 5- or 6-membered heteroaryl and 5- or 6-membered heteroaryl-C₁₋₃ alkyl- is optionally substituted by one or more R¹⁵;
L¹ is a bond or is selected from —O—, —S(O)ₓ-(wherein x is 0, 1, or 2), —NR^{a7}—, —C(O)—, —OC(O)—, —C(O)O—, —NR^{a7}C(O)—, —C(O)NR^{a7}—, —NR^{a7}C(O)O—, —OC(O)NR^{a7}—, —NR^{a7}SO₂—, —SO₂NR^{a7}— and —NR^{a7}C(O)NR^{a7}—;
R¹², R¹³ and R¹⁴ are at each occurrence independently selected from: halo, ═O, —CN, —NO₂, C₁₋₄ alkyl, C₁₋₄ haloalkyl, —OR^{a8}, —S(O)₂R^{a8}, —NR^{a8}R^{b8}, —C(O)R^{a8}, —OC(O)R^{a8}, —C(O)OR^{a8}, —NR^{a8}C(O)R^{b8}, —C(O)NR^{a8}R^{b8}, —NR^{a8}C(O)OR^{b8}, —OC(O)NR^{a8}R^{b8}, —NR^{a8}SO₂R^{b8} and —SO₂NR^{a8}R^{b8},
wherein said C₁₋₄ alkyl is optionally substituted by 1 or 2 substituents selected from: halo, —CN, —OR^{a9}, —NR^{a9}R^{b9} and —SO₂R^{a9};
R¹⁵ is at each occurrence independently selected from: halo, ═O, —CN, —NO₂, C₁₋₄ alkyl, C₁₋₄ haloalkyl, —OR^{a10}, —S(O)₂R^{a10}, —NR^{a10}R^{b10}, —C(O)R^{a10}, —OC(O)R^{a10}, —C(O)OR^{a10}, —NR^{a10}C(O)R^{b10}, —C(O)NR^{a10}R^{b10}, —NR^{a10}C(O)OR^{b10}, —OC(O)NR^{a10}R^{b10}, —NR^{b10}SO₂R^{a10} and —SO₂NR^{a10}R^{b10},
wherein said C₁₋₄ alkyl is optionally substituted by 1 or 2 substituents selected from: halo, —CN, —OR^{a11}, —NR^{a11}R^{b11} and —SO₂R^{a11};
R^{a1}, R^{b1}, R^{a2}, R^{b2}, R^{a3}, R^{b3}, R^{a4}, R^{b4}, R^{a5}, R^{b5}, R^{a6}, R^{a7}, R^{a8}, R^{b8}, R^{a9}, R^{b9}, R^{a10}, R^{b10}, R^{a11} and R^{b1}1 are at each occurrence independently selected from: H, C₁₋₄ alkyl and C₁₋₄ haloalkyl, or any —NR^{a1}R^{b1}, —NR^{a2}R^{b2}, —NR^{a3}R^{b3}, —NR^{a4}R^{b4}, —NR^{a5}R^{b5}, —NR^{a8}R^{b8}, —NR^{a9}R^{b9}, —NR^{a10}R^{b10}, NR^{a11}R^{b11}, —NR⁵R⁶, —NR⁸R⁹ or —NR¹¹R^{a6} within a substituent may form a 4- to 6-membered heterocyclyl, wherein said 4- to 6-membered heterocyclyl is optionally substituted by one or more substituents selected from: halo, ═O, C₁₋₄ alkyl and C₁₋₄ haloalkyl,
with the provisos (i) and (ii):
(i) when Ring A is A2, and R² is H, then R³ is not —NR⁵R⁶; and
(ii) the compound of Formula (I) is not

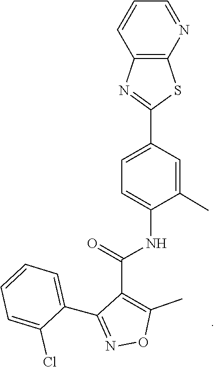 [SMILES: Cc1cc(-c2nc3cccnc3s2)ccc1NC(=O)c1c(-c2ccccc2Cl)noc1C]

2. The compound according to claim 1, wherein R² is C₁₋₄ alkyl, for example wherein R² is methyl.

3. The compound according to claim 1, wherein R¹ is: 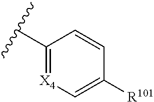
wherein:
X⁴ is CH or N; and
R¹⁰¹ is H or halo.

4. The compound according to claim 1, wherein Ring A has a structure selected from:

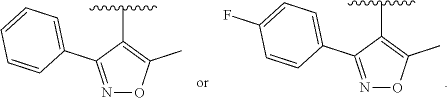 [SMILES: *c1c(-c2ccccc2)noc1C ; *c1c(-c2ccc(F)cc2)noc1C]

5. The compound according to claim 1, wherein X₁ and X₂ are CH.

6. The compound according to claim 1, wherein X₃ is N.

7. The compound according to claim 1, wherein X₂ and X₃ are N and X₁ is CR⁷.

8. The compound according to claim 1, wherein R³ is selected from: —OR⁴ and —NR⁵R⁶.

9. The compound according to claim 1, wherein:
(i) R³ is —OR⁴, wherein R⁴ is selected from: C₁₋₄ alkyl and C₃₋₆ cycloalkyl; or
(ii) R³ is —OR⁴, wherein R⁴ is 4- to 6-membered heterocyclyl containing 1 ring oxygen atom, or 4- to 6-membered heterocyclyl-C₁₋₃ alkyl-containing 1 ring oxygen atom; or
(iii) R³ is selected from: methoxy, 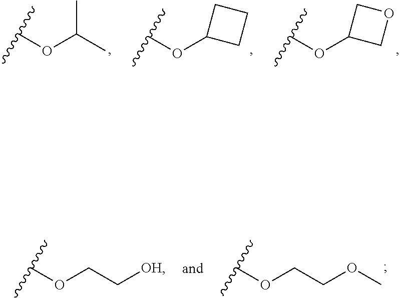
or
(iv) R³ is 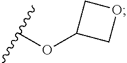 [SMILES: *OC1COC1]
or
(v) R³ is selected from: methoxy and 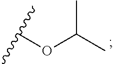 [SMILES: *OC(C)C]
or
(vi) R³ is methoxy.

10. The compound according to claim 1, wherein:
(i) Ring B is selected from: phenyl and a monocyclic or bicyclic 5- to 12-membered heteroaryl, each of which is optionally substituted with one or more R¹⁰; or
(ii) Ring B is selected from: a 5- or 6-membered heteroaryl, each of which is optionally substituted with one or more R¹⁰; or
(iii) Ring B is selected from: a bicyclic 9- or 10-membered heteroaryl optionally substituted with one or more R¹⁰.

11. The compound according to claim 1, wherein Ring B is selected from: 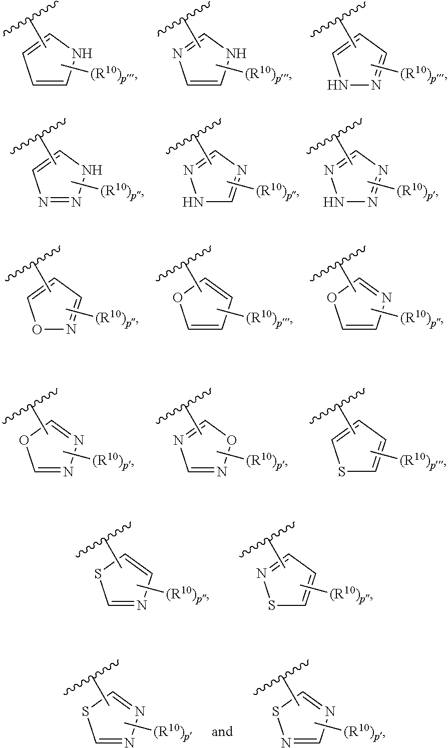
wherein p′ is 0 or 1; p″ is 0, 1 or 2; p′″ is 0, 1, 2 or 3; and [symbol]
indicates the point of attachment to the remainder of the compound of Formula (I).

12. The compound according to claim 1, wherein Ring B is bonded to the remainder of the compound of Formula (I) via a ring carbon atom in an aromatic ring in Ring B.

13. The compound according to claim 1, wherein Ring B is bonded to the remainder of the compound of Formula (I) via a ring nitrogen atom in an aromatic ring in Ring B.

14. The compound according to claim 1, wherein:
(i) Ring B is: 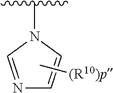
wherein p″ is 0, 1 or 2; or
(ii) Ring B is selected from: 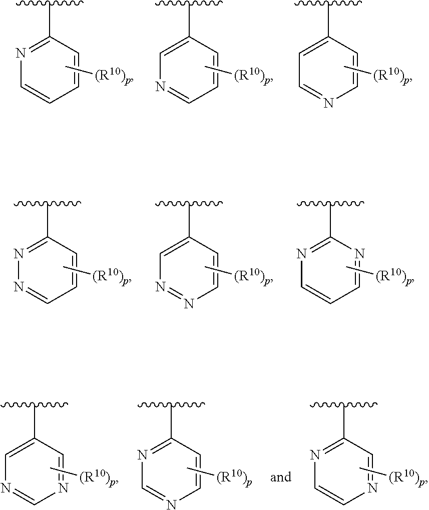
wherein p is 0, 1, 2 or 3, and wherein [symbol] indicates the point of attachment to the remainder of the compound of Formula (I); or
(iii) Ring B is selected from: 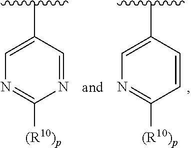
wherein p is 0 or 1; and [symbol] indicates the point of attachment to the remainder of the compound of Formula (I); or
(iv) Ring B is: 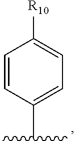
wherein [symbol] indicates the point of attachment to the remainder of the compound of Formula (I).

15. The compound according to claim 1, wherein:
(i) R¹⁰ at each occurrence is independently selected from: halo, —CN, C₁₋₄ alkyl, C₁₋₄ haloalkyl, —C₁₋₄ alkyl-NR^{a8}R^{b8}, —OH, —OC₁₋₄ alkyl, —OC₁₋₄ haloalkyl, —OC₂₋₄ alkyl-NR^{a8}R^{b8}, —NH₂, —NR²⁶C₁₋₄ alkyl, —NR^{a6}C₂₋₄ alkyl-OR^{a8}, —NR^{a6}C₂₋₄ alkyl-NR^{a8}R^{b8}, —C(O)C₁₋₄ alkyl, —C(O)C₁₋₄ haloalkyl, —C(O)C₁₋₄ alkyl-NR^{a8}R^{b8}, —COOH, —C(O)OC₁₋₄ alkyl, —C(O)NR^{a6}C₁₋₄ alkyl, —C(O)NR²⁶C₂₋₄ alkyl-OR^{a8}, —C(O)NR^{a6}C₂₋₄ alkyl-NR^{a8}R^{b8} and Q¹⁰²-L¹⁰²-;
wherein Q¹⁰² is selected from 4- to 6-membered heterocyclyl and 5- or 6-membered heteroaryl,
wherein said 4- to 6-membered heterocyclyl is selected from: azetidinyl, pyrrolidinyl, piperidinyl, piperazinyl and morpholinyl, each of which is optionally substituted by one or more (e.g. 1 or 2) substituents selected from: halo, ═O, C₁₋₄ alkyl, —OR^{a8}, —NR^{a8}R^{b8} and —C(O)R^{a8},
wherein said 5- or 6-membered heteroaryl is selected from: pyrrolyl, imidazolyl, oxazolyl, isoxazolyl, thiazolyl, isothiazolyl, pyrazolyl, oxadiazolyl, triazolyl, pyridyl, pyrimidyl, pyrazinyl and pyridazinyl, each of which is optionally substituted by one or more (e.g. 1 or 2) substituents selected from: halo, C₁₋₄ alkyl, —OR^{a10} and —NR^{a10}R^{b10};
L¹⁰² is a bond or is selected from: C₁₋₃ alkylene, —O— and —NR^{a7}—; or
(ii) R¹⁰ is selected from: —NR^{a81}R^{b81}, —C₁₋₃ alkyl-NR^{a81}R^{b81}, —NR^{a81}—C₂₋₃ alkyl-NR^{a81}R^{b81}, —C(O)C₁₋₃ alkyl-NR^{a81}R^{b81}, and —C(O)NR^{a81}C₂₋₃ alkyl-NR^{a81}R^{b81}, wherein R^{a81} and R^{b81} are independently selected from H and C₁₋₃ alkyl; or
(iii) R¹⁰ is selected from: —NR^{a81}R^{b81} and —C₁₋₃ alkyl-NR^{a81}R^{b81}, wherein R^{a81} and R^{b81} are independently selected from H and C₁₋₃ alkyl; or
(iv) R¹⁰ is selected from: fluoro, chloro, cyano, nitro, oxo, hydroxy methyl, ethyl, isopropyl, cyclopropyl, amino, —NH(Me), —N(Me)₂, 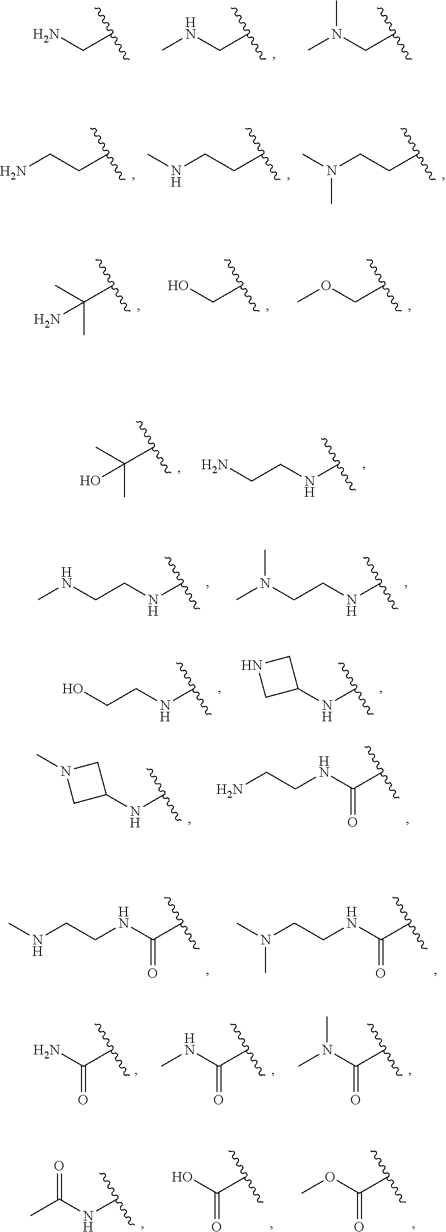 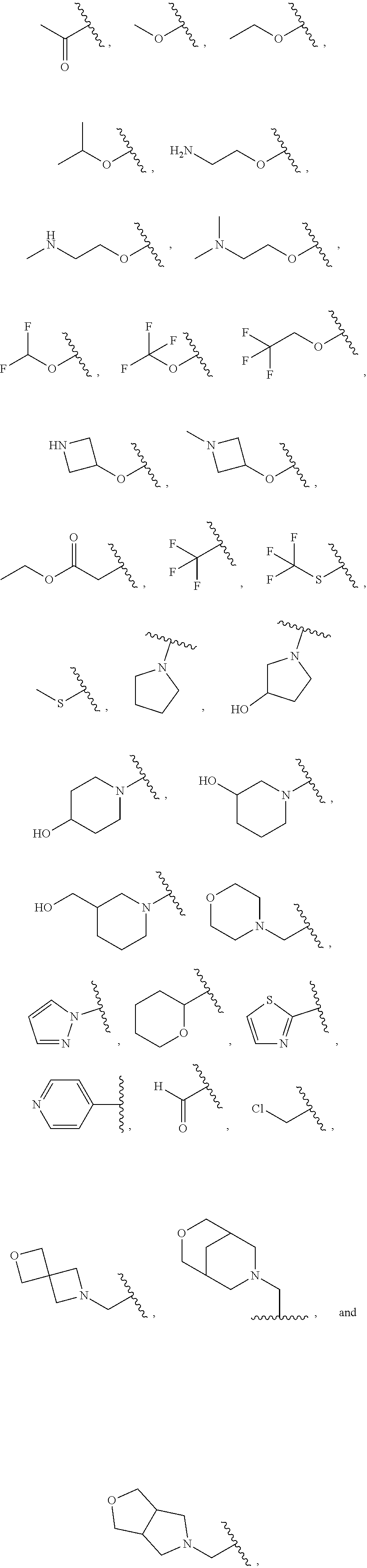
wherein [symbol] indicates the point of attachment to Ring B.

16. The compound according to claim 1, wherein Ring B is selected from:

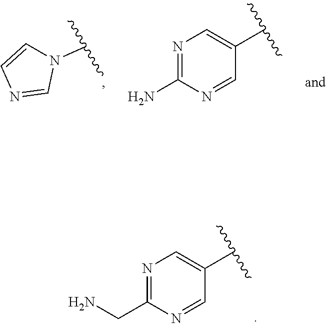 [SMILES: *n1ccnc1 ; *c1cnc(N)nc1 ; *c1cnc(CN)nc1]

17. The compound according to claim 1, wherein the compound is selected from 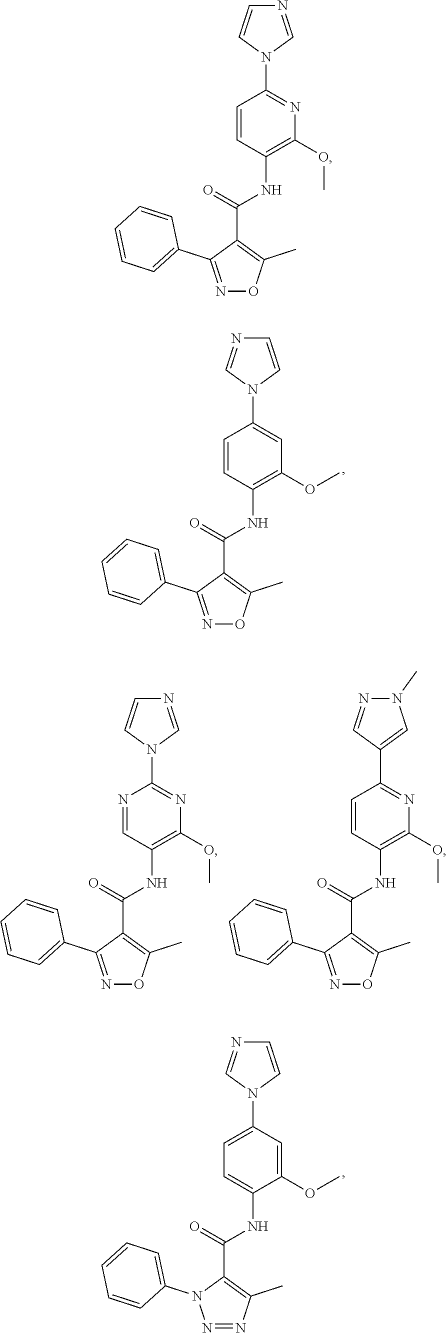 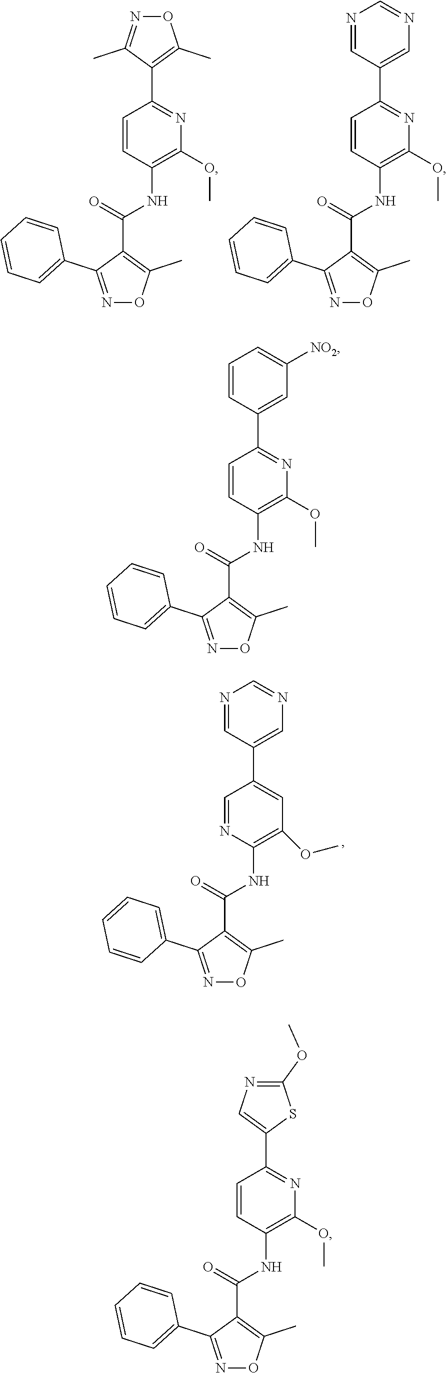 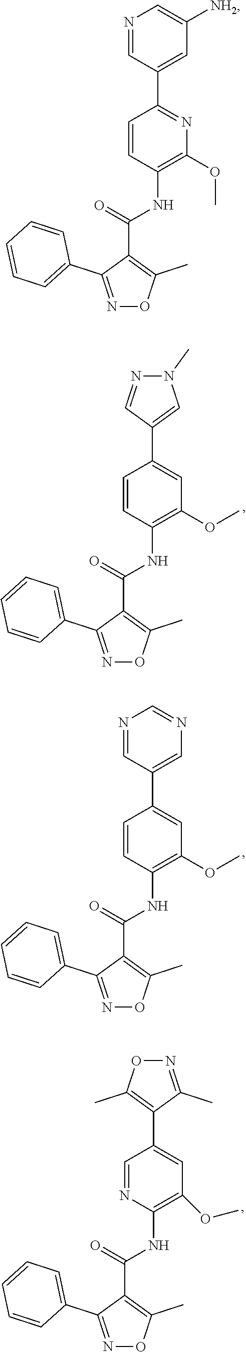 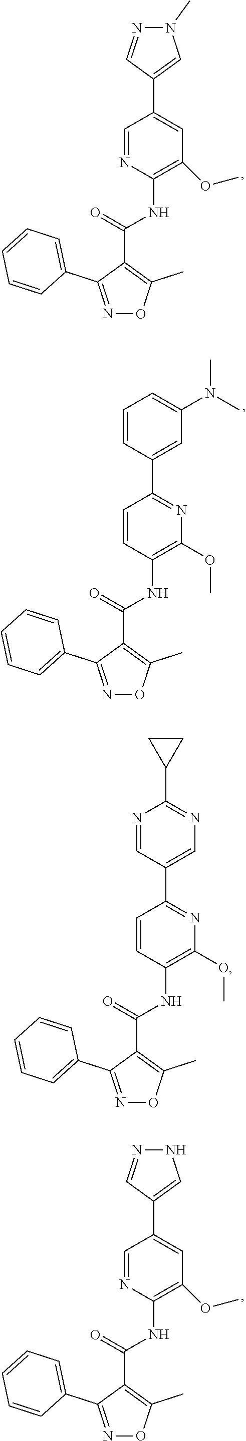 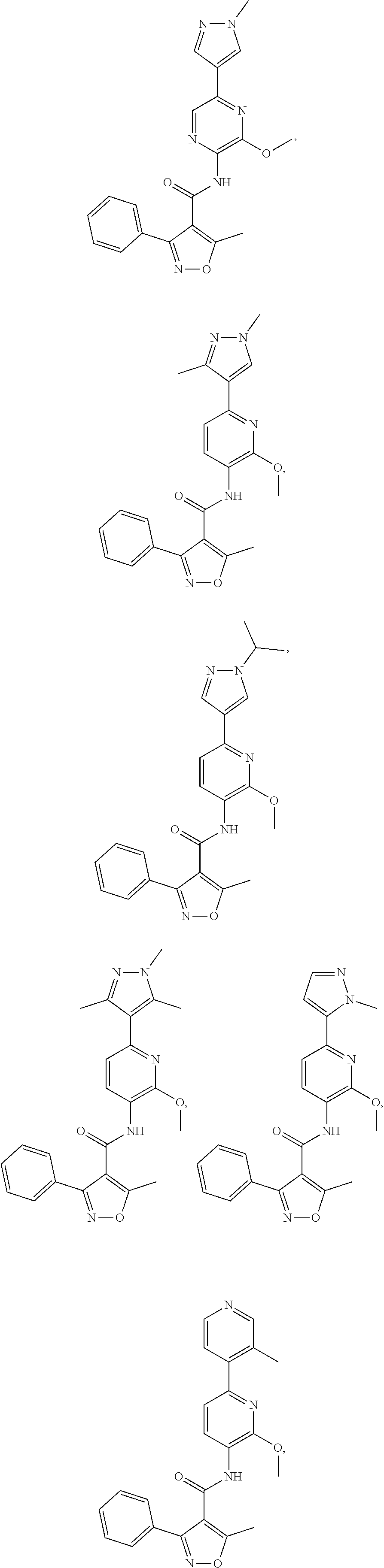 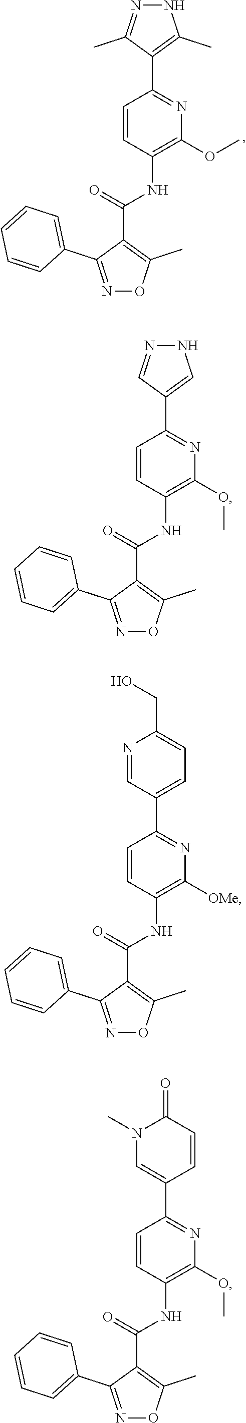 [SMILES: COc1nc(-c2c(C)n[nH]c2C)ccc1NC(=O)c1c(-c2ccccc2)noc1C ; COc1nc(-c2cn[nH]c2)ccc1NC(=O)c1c(-c2ccccc2)noc1C ; COc1nc(-c2ccc(CO)nc2)ccc1NC(=O)c1c(-c2ccccc2)noc1C ; COc1nc(-c2ccc(=O)n(C)c2)ccc1NC(=O)c1c(-c2ccccc2)noc1C] 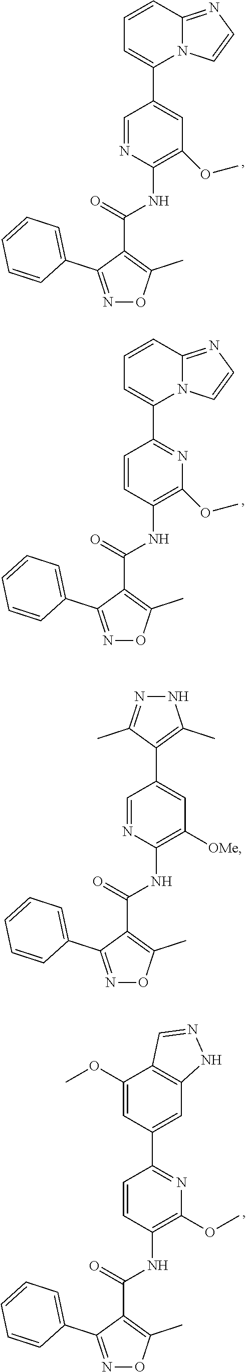 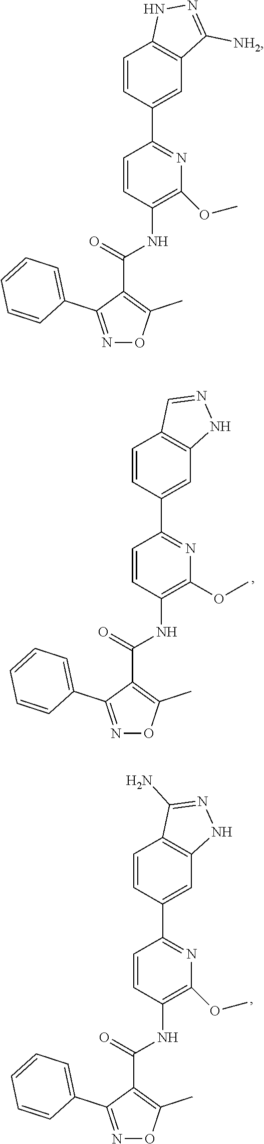 [SMILES: COc1nc(-c2ccc3[nH]nc(N)c3c2)ccc1NC(=O)c1c(-c2ccccc2)noc1C ; COc1nc(-c2ccc3cn[nH]c3c2)ccc1NC(=O)c1c(-c2ccccc2)noc1C ; COc1nc(-c2ccc3c(N)n[nH]c3c2)ccc1NC(=O)c1c(-c2ccccc2)noc1C] 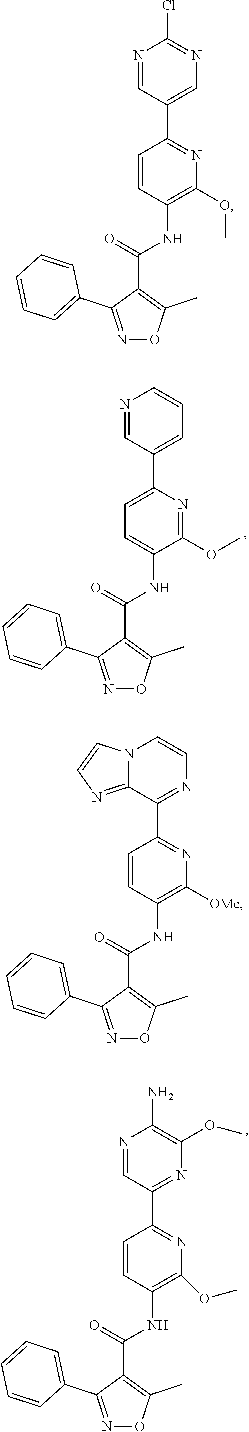 [SMILES: COc1nc(-c2cnc(Cl)nc2)ccc1NC(=O)c1c(-c2ccccc2)noc1C ; COc1nc(-c2cccnc2)ccc1NC(=O)c1c(-c2ccccc2)noc1C ; COc1nc(-c2nccn3ccnc23)ccc1NC(=O)c1c(-c2ccccc2)noc1C ; COc1nc(-c2cnc(N)c(OC)n2)ccc1NC(=O)c1c(-c2ccccc2)noc1C] 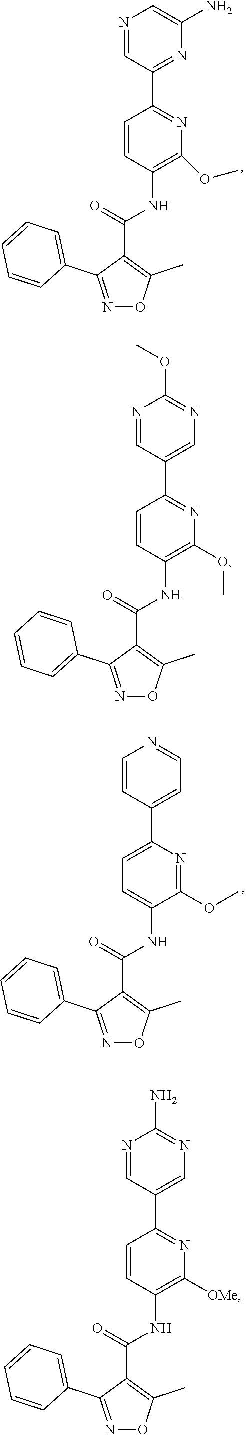 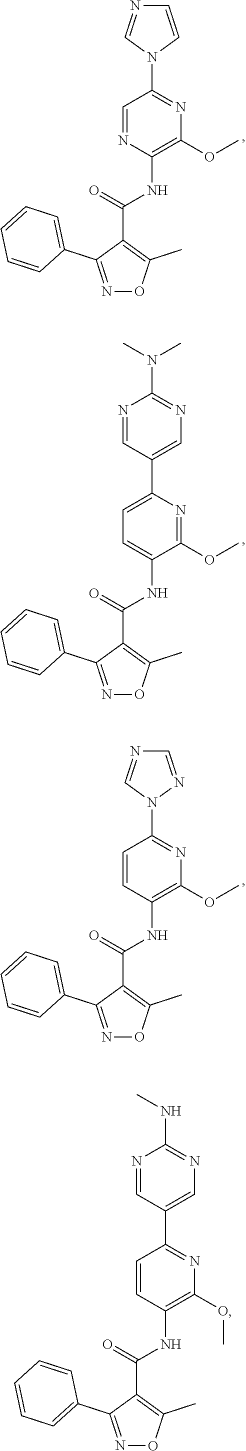 [SMILES: COc1nc(-n2ccnc2)cnc1NC(=O)c1c(-c2ccccc2)noc1C ; COc1nc(-c2cnc(N(C)C)nc2)ccc1NC(=O)c1c(-c2ccccc2)noc1C ; COc1nc(-n2cncn2)ccc1NC(=O)c1c(-c2ccccc2)noc1C ; CNc1ncc(-c2ccc(NC(=O)c3c(-c4ccccc4)noc3C)c(OC)n2)cn1] 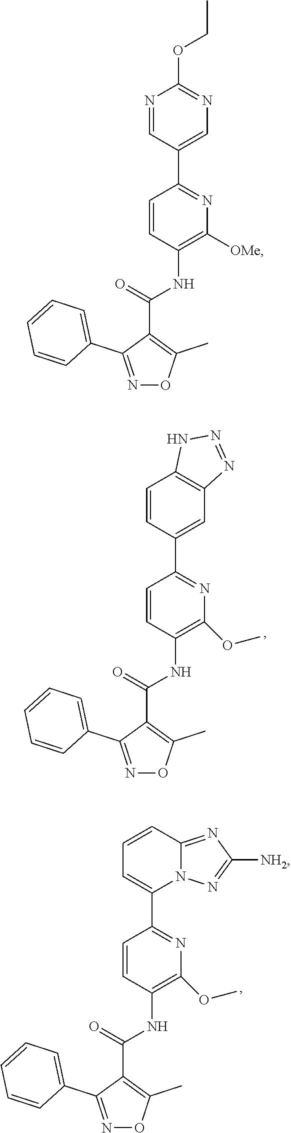 [SMILES: CCOc1ncc(-c2ccc(NC(=O)c3c(-c4ccccc4)noc3C)c(OC)n2)cn1 ; COc1nc(-c2ccc3[nH]nnc3c2)ccc1NC(=O)c1c(-c2ccccc2)noc1C ; COc1nc(-c2cccc3nc(N)nn23)ccc1NC(=O)c1c(-c2ccccc2)noc1C] 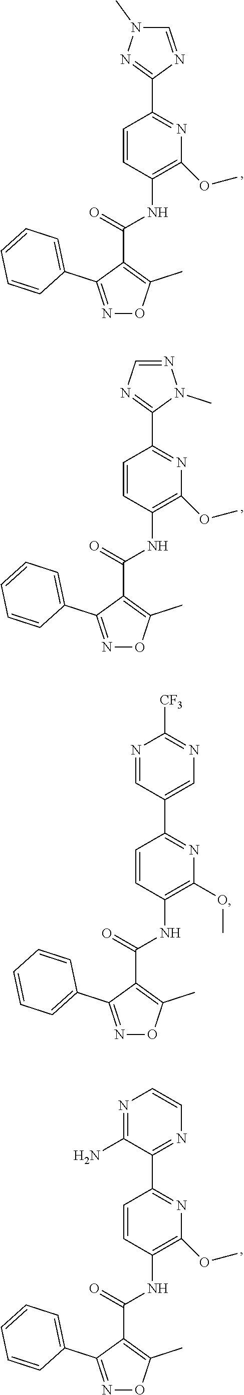 [SMILES: COc1nc(-c2ncn(C)n2)ccc1NC(=O)c1c(-c2ccccc2)noc1C ; COc1nc(-c2ncnn2C)ccc1NC(=O)c1c(-c2ccccc2)noc1C ; COc1nc(-c2cnc(C(F)(F)F)nc2)ccc1NC(=O)c1c(-c2ccccc2)noc1C ; COc1nc(-c2nccnc2N)ccc1NC(=O)c1c(-c2ccccc2)noc1C] 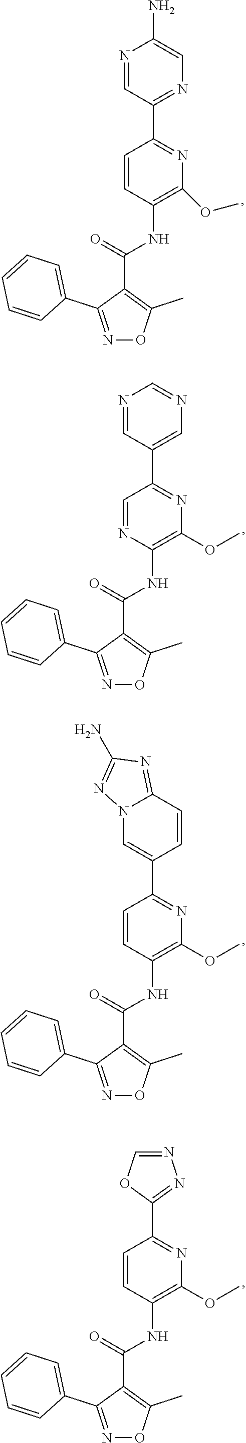 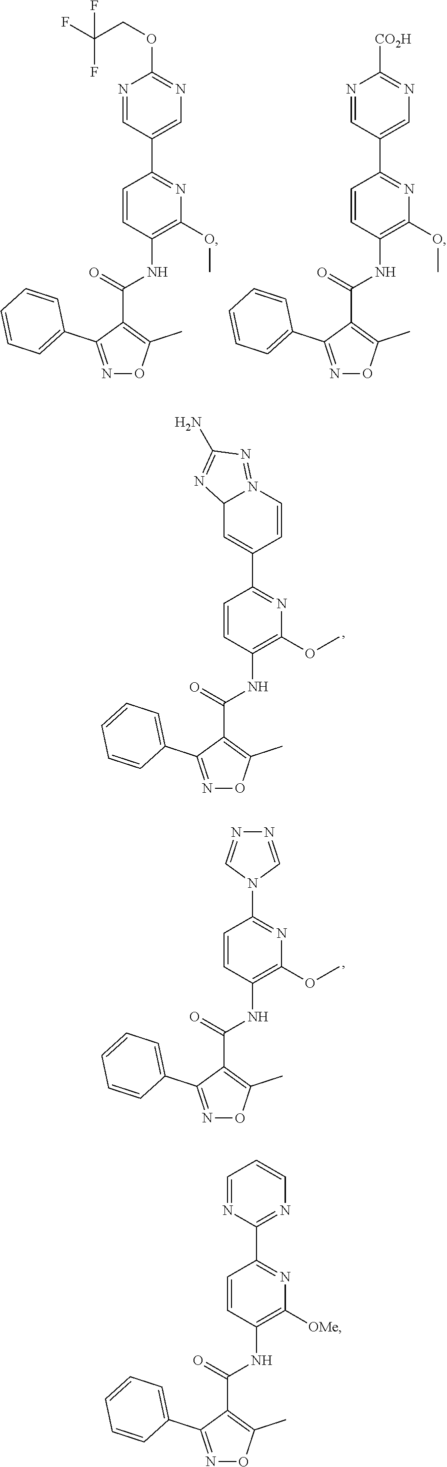 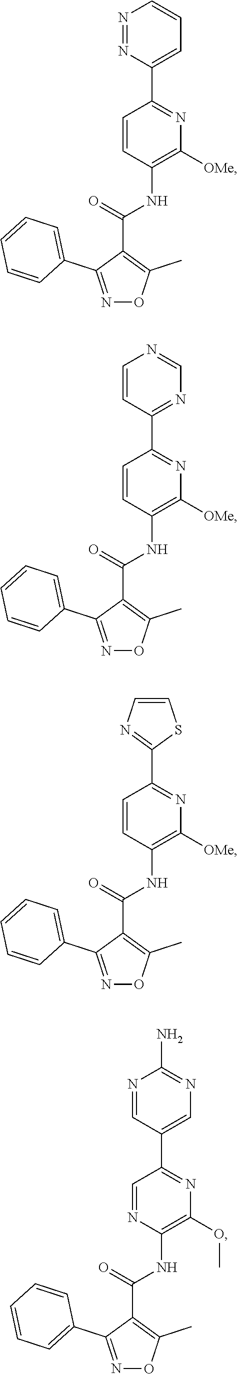 [SMILES: COc1nc(-c2cccnn2)ccc1NC(=O)c1c(-c2ccccc2)noc1C ; COc1nc(-c2ccncn2)ccc1NC(=O)c1c(-c2ccccc2)noc1C ; COc1nc(-c2nccs2)ccc1NC(=O)c1c(-c2ccccc2)noc1C ; COc1nc(-c2cnc(N)nc2)cnc1NC(=O)c1c(-c2ccccc2)noc1C] 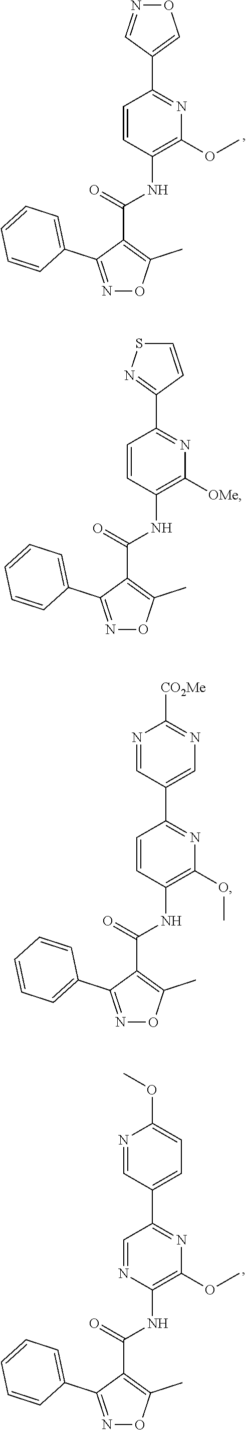 [SMILES: COc1nc(-c2cnoc2)ccc1NC(=O)c1c(-c2ccccc2)noc1C ; COc1nc(-c2ccsn2)ccc1NC(=O)c1c(-c2ccccc2)noc1C ; COC(=O)c1ncc(-c2ccc(NC(=O)c3c(-c4ccccc4)noc3C)c(OC)n2)cn1 ; COc1ccc(-c2cnc(NC(=O)c3c(-c4ccccc4)noc3C)c(OC)n2)cn1] 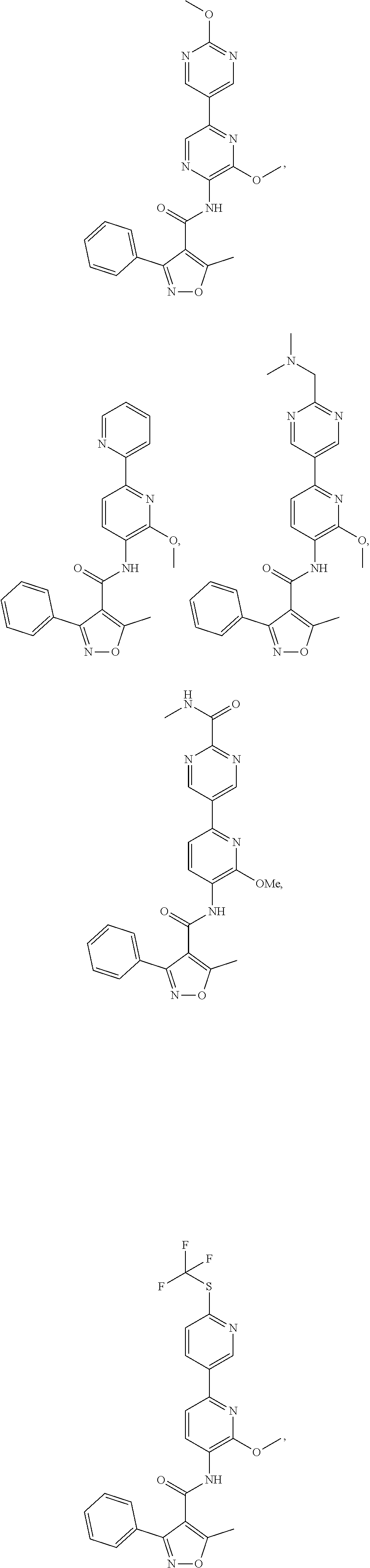 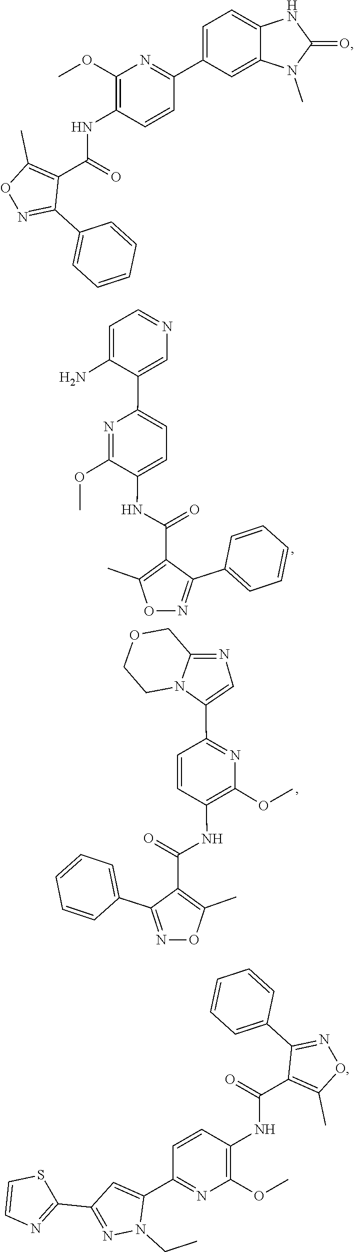 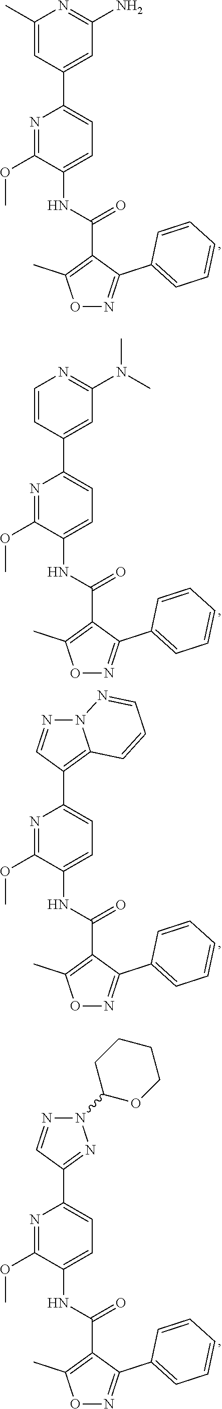 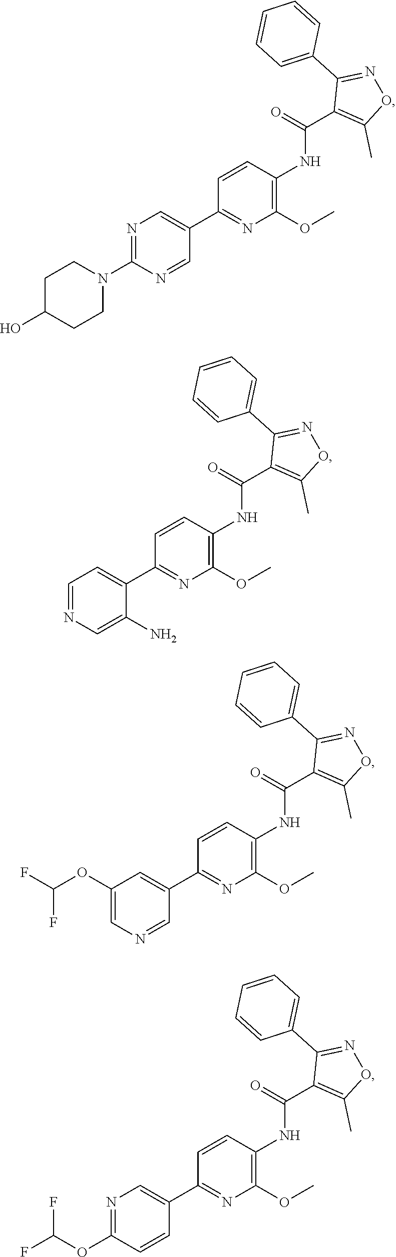 [SMILES: COc1nc(-c2cnc(N3CCC(O)CC3)nc2)ccc1NC(=O)c1c(-c2ccccc2)noc1C ; COc1nc(-c2ccncc2N)ccc1NC(=O)c1c(-c2ccccc2)noc1C ; COc1nc(-c2cncc(OC(F)F)c2)ccc1NC(=O)c1c(-c2ccccc2)noc1C ; COc1nc(-c2ccc(OC(F)F)nc2)ccc1NC(=O)c1c(-c2ccccc2)noc1C] 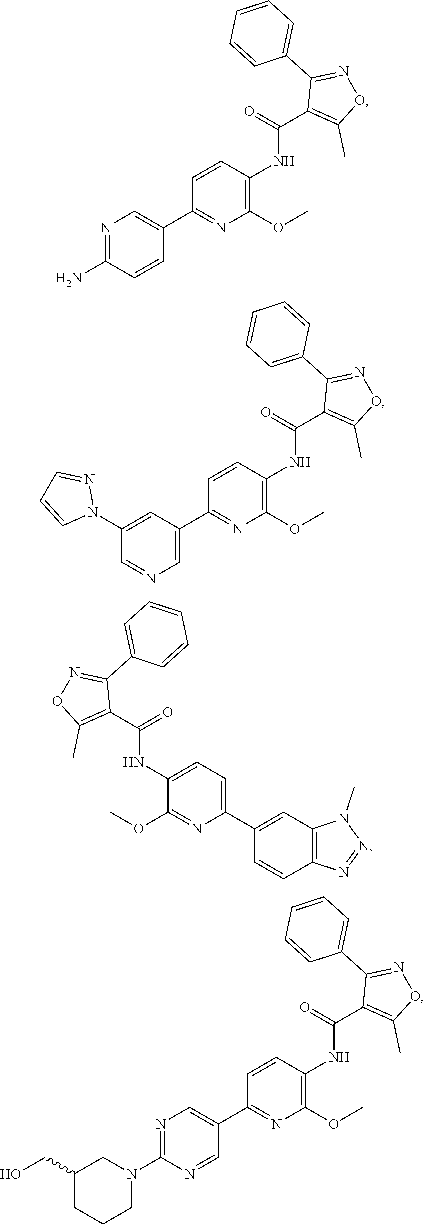 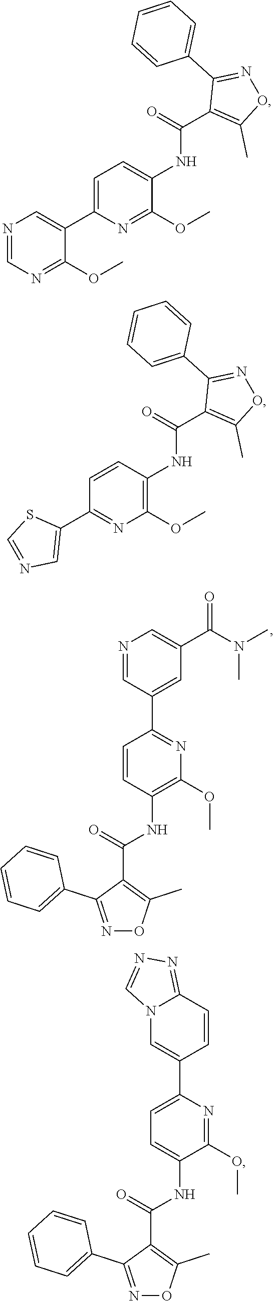 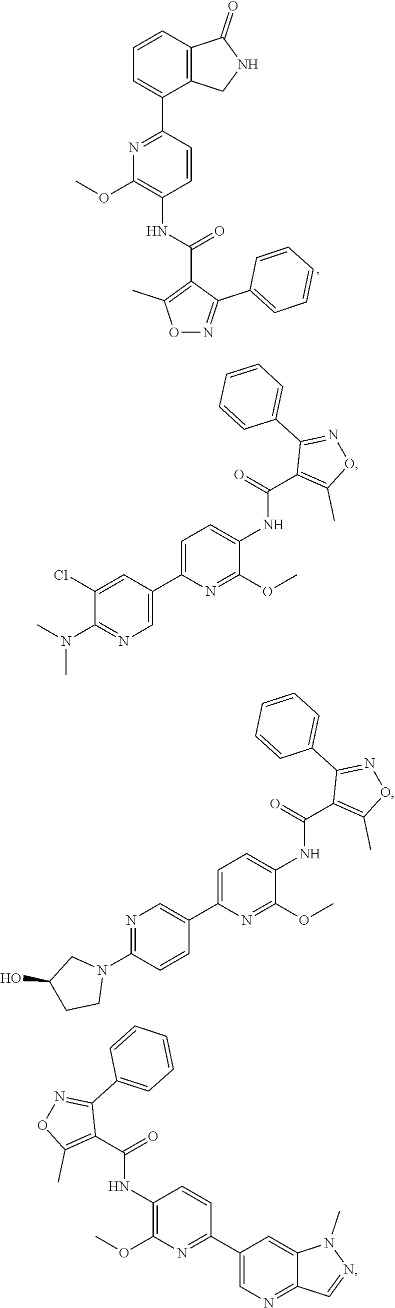 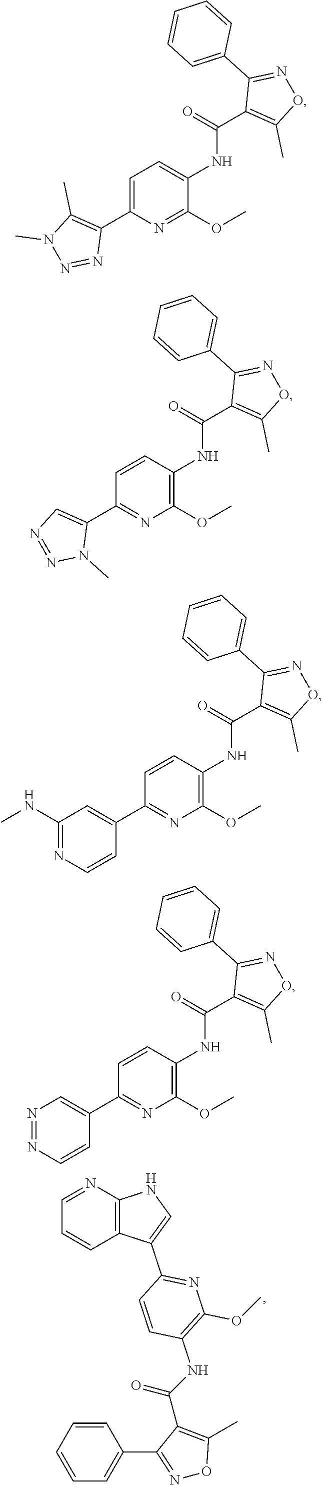 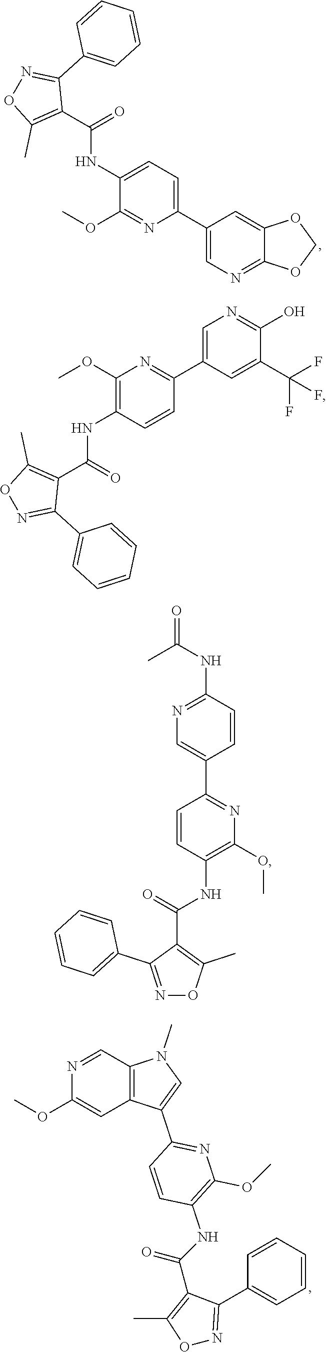 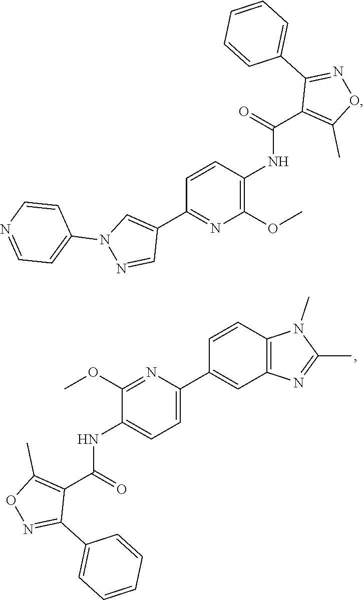 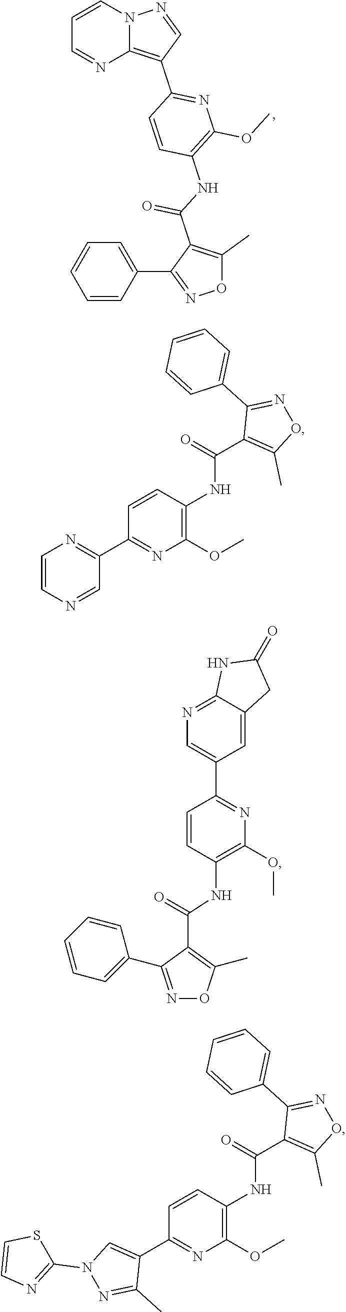 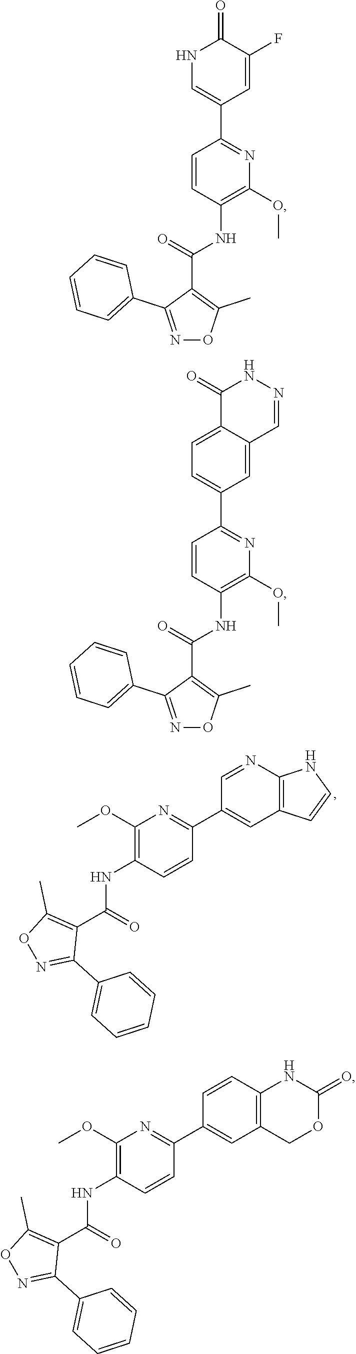 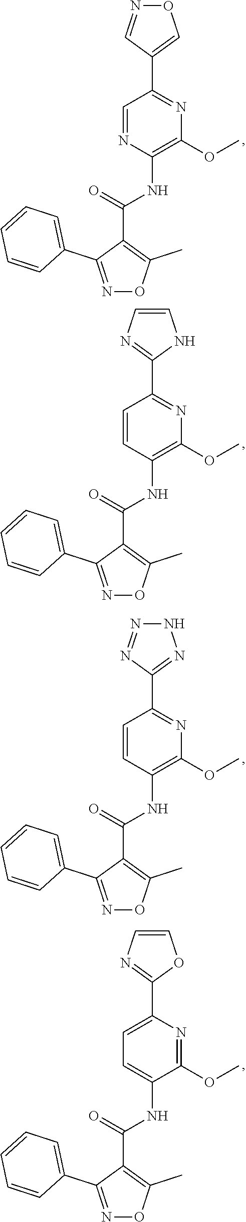 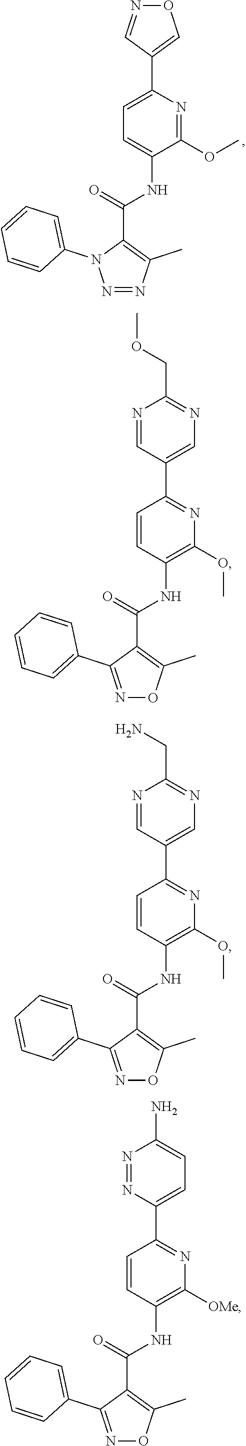 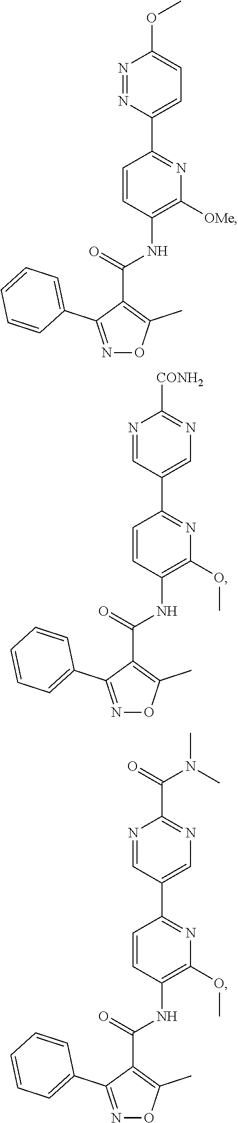 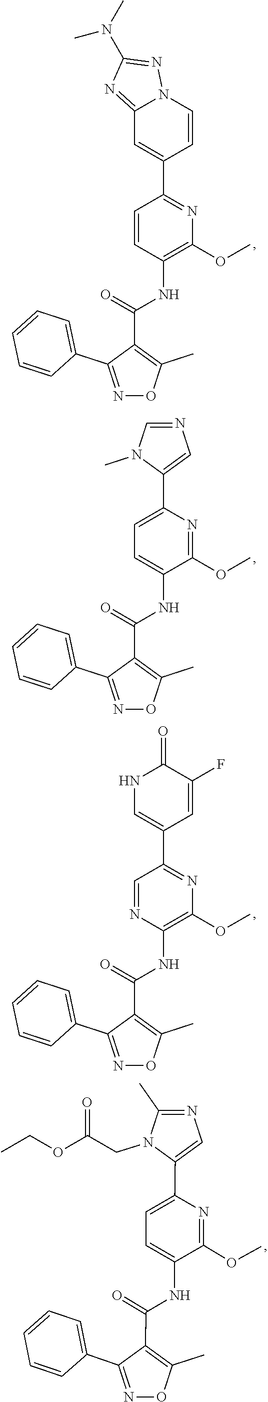 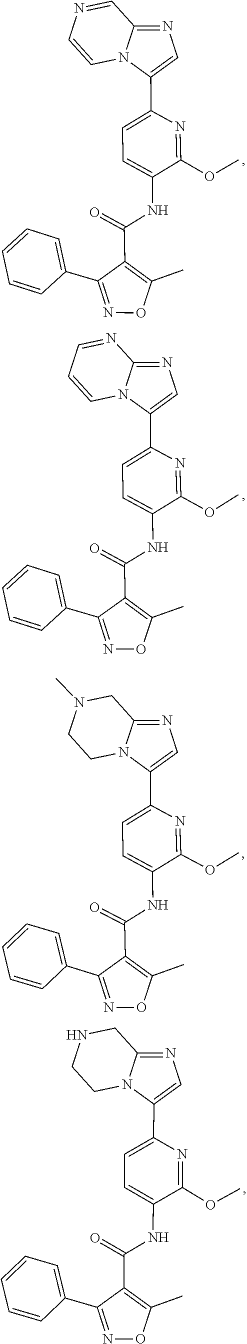 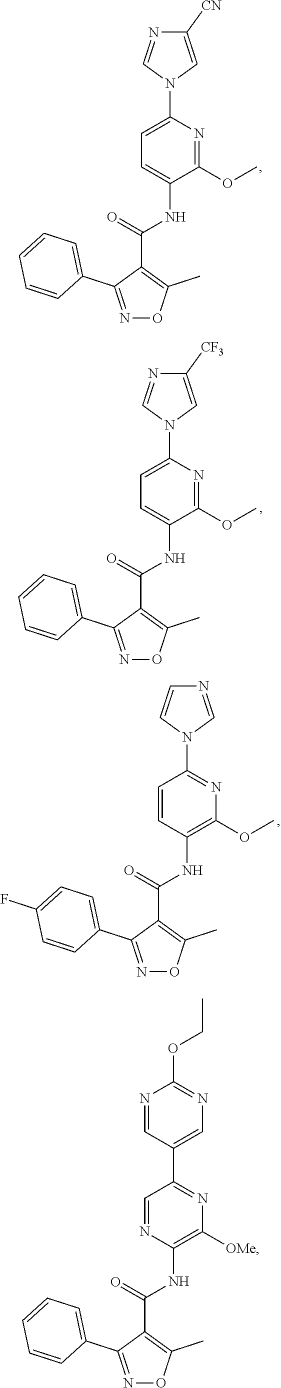 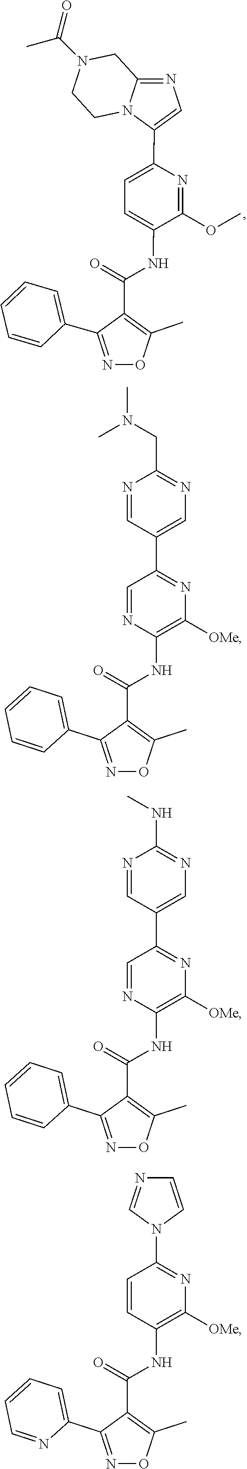 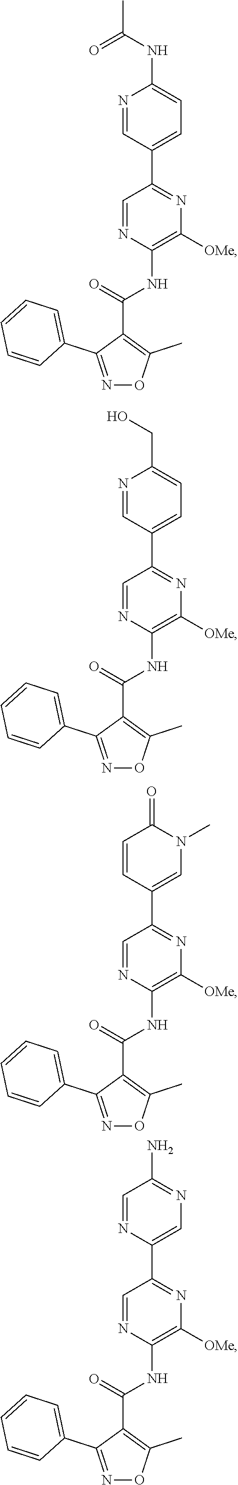 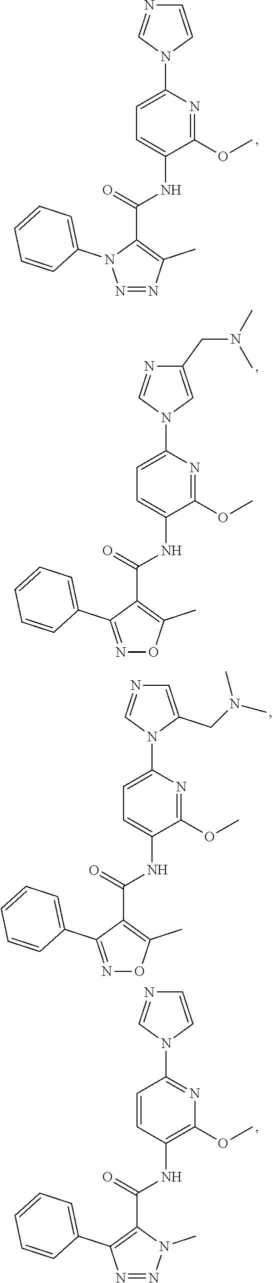 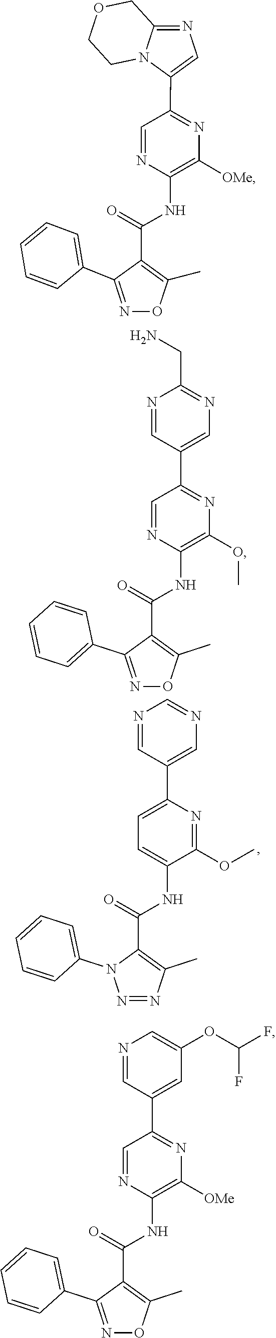 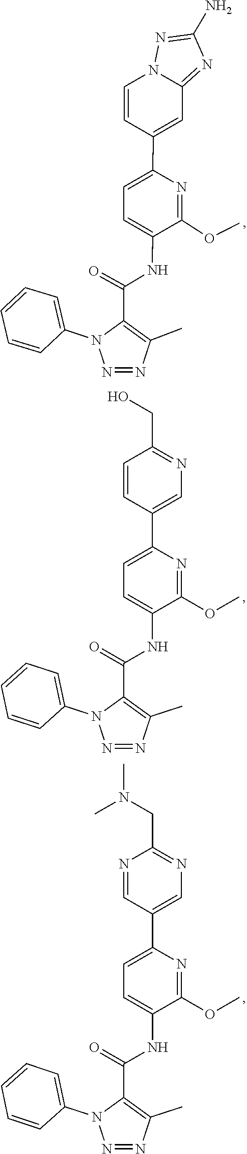 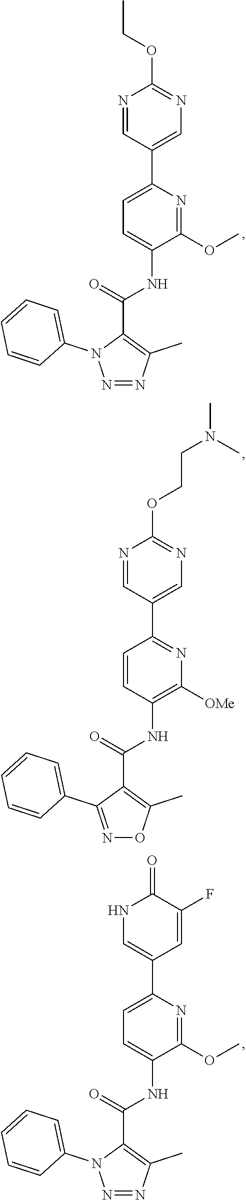 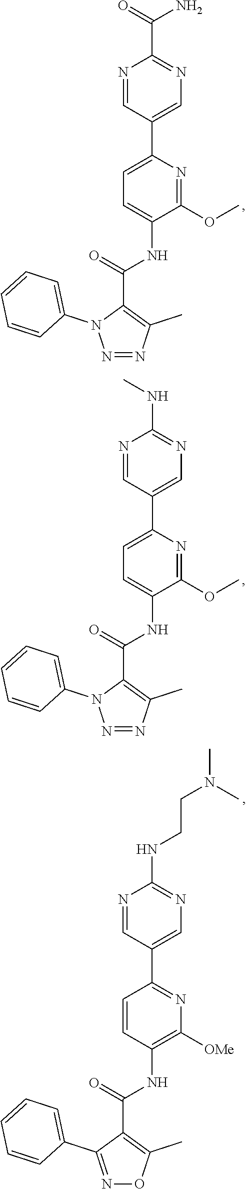 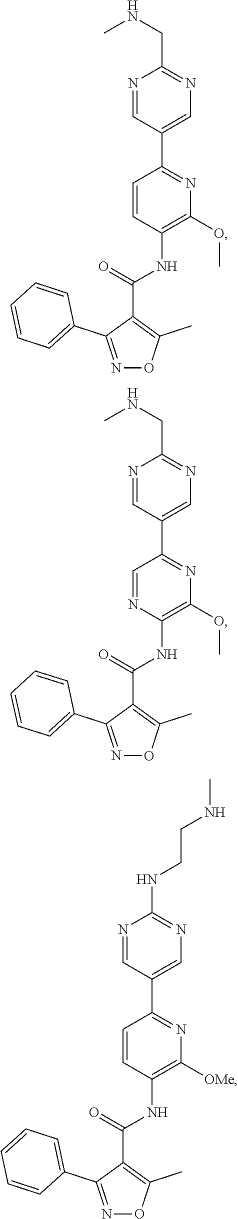 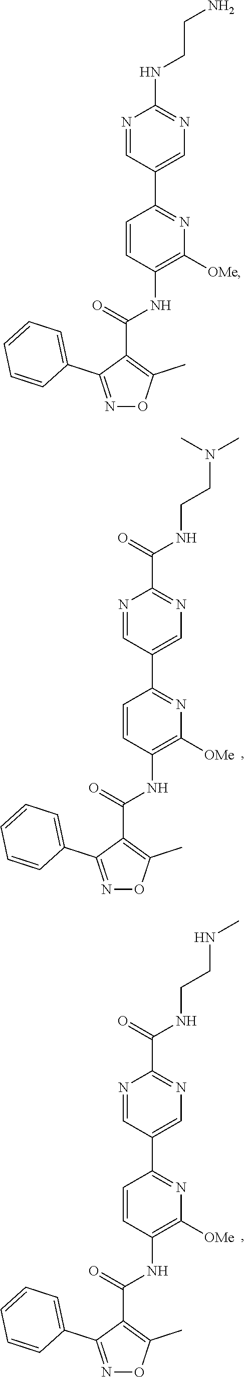 [SMILES: COc1nc(-c2cnc(NCCN)nc2)ccc1NC(=O)c1c(-c2ccccc2)noc1C ; COc1nc(-c2cnc(C(=O)NCCN(C)C)nc2)ccc1NC(=O)c1c(-c2ccccc2)noc1C ; CNCCNC(=O)c1ncc(-c2ccc(NC(=O)c3c(-c4ccccc4)noc3C)c(OC)n2)cn1] 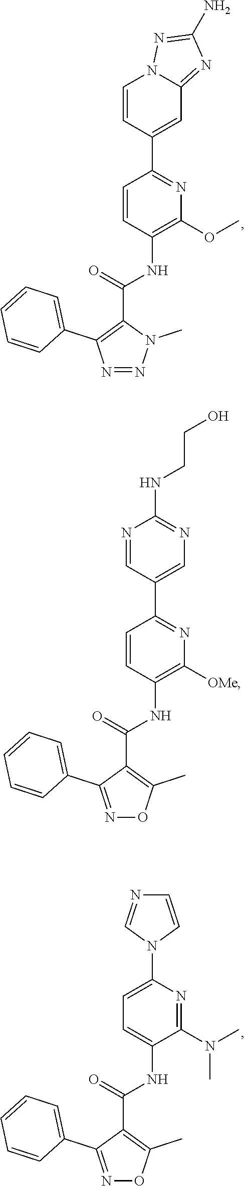 [SMILES: COc1nc(-c2ccn3nc(N)nc3c2)ccc1NC(=O)c1c(-c2ccccc2)nnn1C ; COc1nc(-c2cnc(NCCO)nc2)ccc1NC(=O)c1c(-c2ccccc2)noc1C ; Cc1onc(-c2ccccc2)c1C(=O)Nc1ccc(-n2ccnc2)nc1N(C)C] 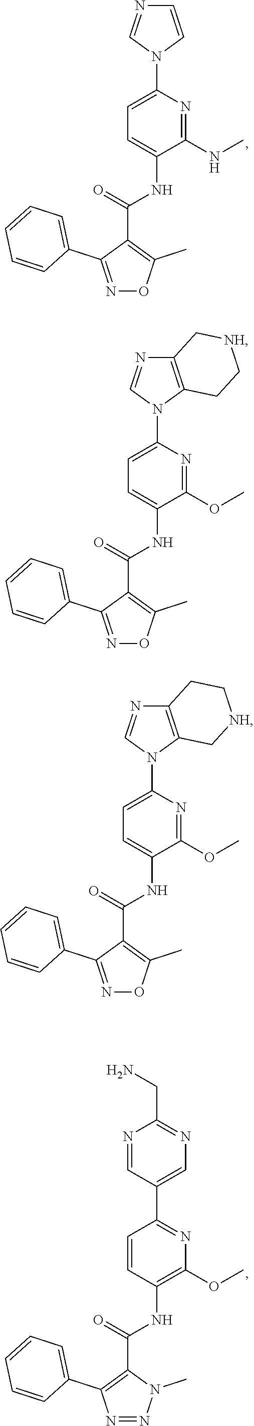 [SMILES: CNc1nc(-n2ccnc2)ccc1NC(=O)c1c(-c2ccccc2)noc1C ; COc1nc(-n2cnc3c2CCNC3)ccc1NC(=O)c1c(-c2ccccc2)noc1C ; COc1nc(-n2cnc3c2CNCC3)ccc1NC(=O)c1c(-c2ccccc2)noc1C ; COc1nc(-c2cnc(CN)nc2)ccc1NC(=O)c1c(-c2ccccc2)nnn1C] 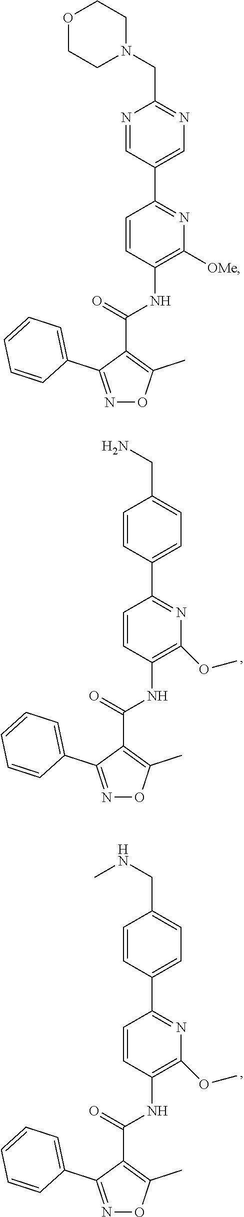 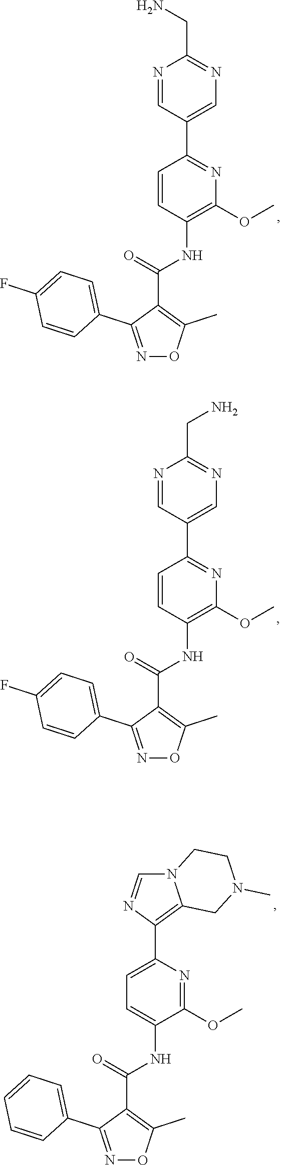 [SMILES: COc1nc(-c2cnc(CN)nc2)ccc1NC(=O)c1c(-c2ccc(F)cc2)noc1C ; COc1nc(-c2cnc(CN)nc2)ccc1NC(=O)c1c(-c2ccc(F)cc2)noc1C ; COc1nc(-c2ncn3c2CN(C)CC3)ccc1NC(=O)c1c(-c2ccccc2)noc1C] 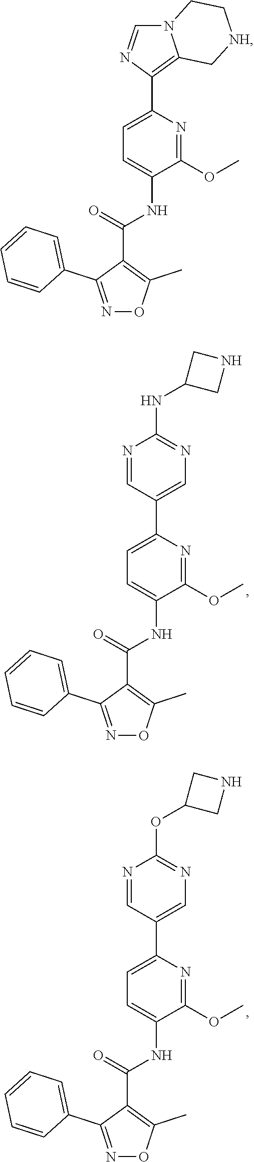 [SMILES: COc1nc(-c2ncn3c2CNCC3)ccc1NC(=O)c1c(-c2ccccc2)noc1C ; COc1nc(-c2cnc(NC3CNC3)nc2)ccc1NC(=O)c1c(-c2ccccc2)noc1C ; COc1nc(-c2cnc(OC3CNC3)nc2)ccc1NC(=O)c1c(-c2ccccc2)noc1C] 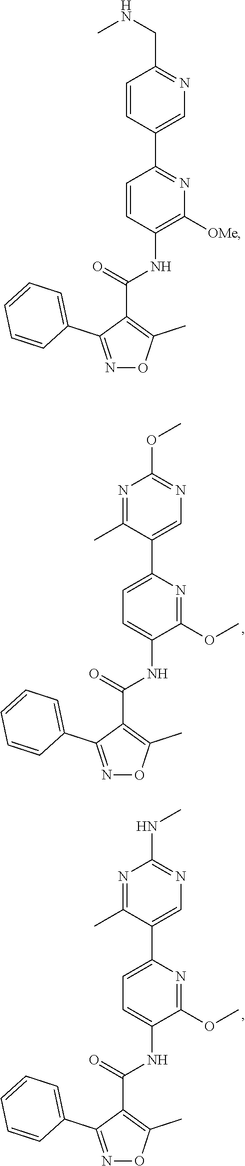 [SMILES: CNCc1ccc(-c2ccc(NC(=O)c3c(-c4ccccc4)noc3C)c(OC)n2)cn1 ; COc1ncc(-c2ccc(NC(=O)c3c(-c4ccccc4)noc3C)c(OC)n2)c(C)n1 ; CNc1ncc(-c2ccc(NC(=O)c3c(-c4ccccc4)noc3C)c(OC)n2)c(C)n1] 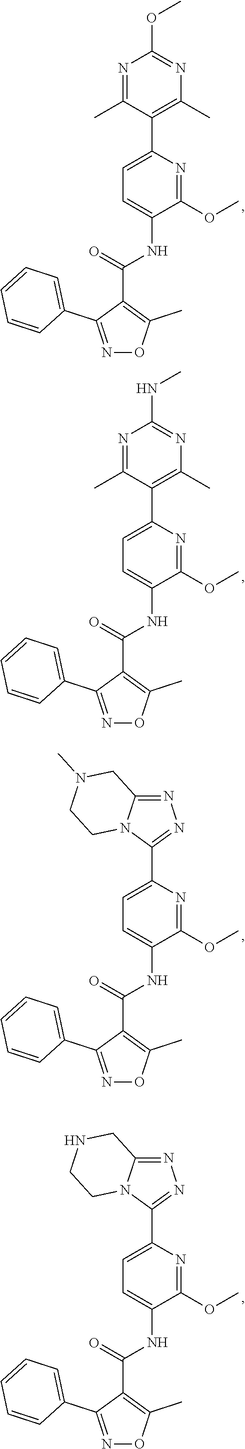 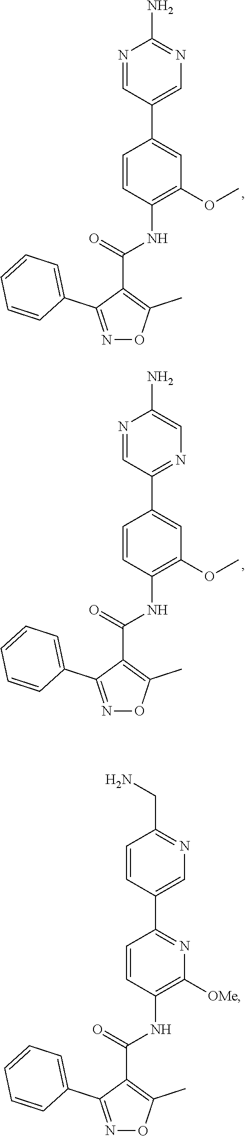 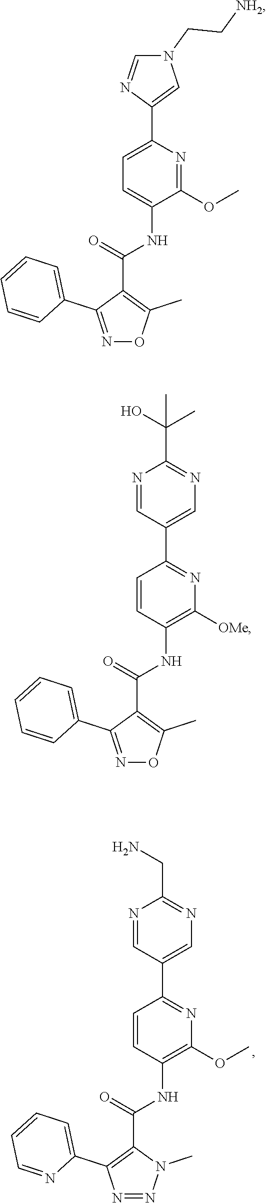 [SMILES: COc1nc(-c2cn(CCN)cn2)ccc1NC(=O)c1c(-c2ccccc2)noc1C ; COc1nc(-c2cnc(C(C)(C)O)nc2)ccc1NC(=O)c1c(-c2ccccc2)noc1C ; COc1nc(-c2cnc(CN)nc2)ccc1NC(=O)c1c(-c2ccccn2)nnn1C] 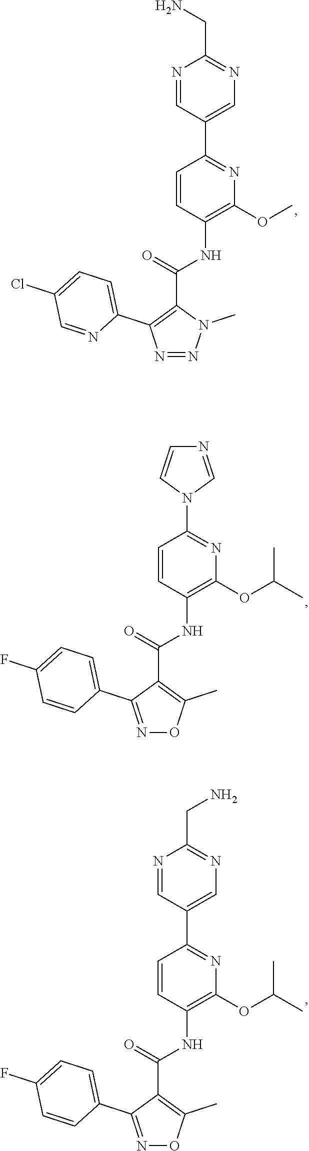 [SMILES: COc1nc(-c2cnc(CN)nc2)ccc1NC(=O)c1c(-c2ccc(Cl)cn2)nnn1C ; Cc1onc(-c2ccc(F)cc2)c1C(=O)Nc1ccc(-n2ccnc2)nc1OC(C)C ; Cc1onc(-c2ccc(F)cc2)c1C(=O)Nc1ccc(-c2cnc(CN)nc2)nc1OC(C)C] 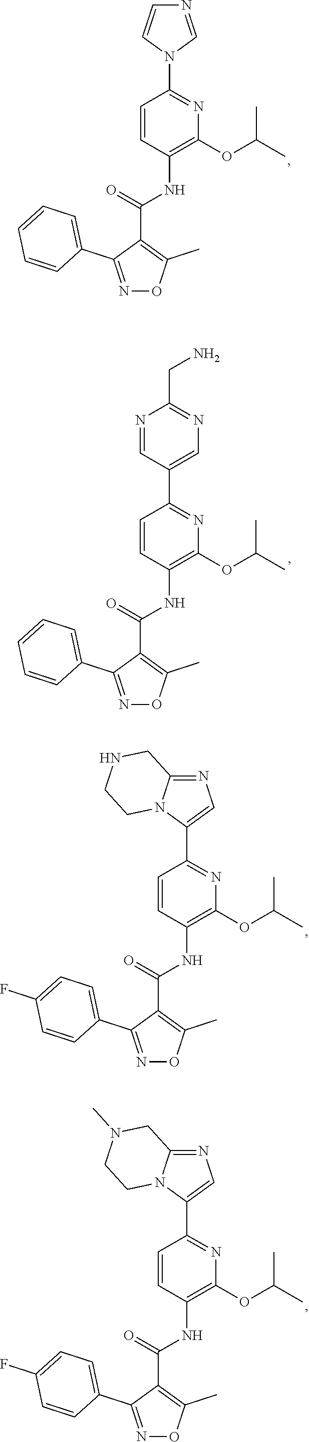 [SMILES: Cc1onc(-c2ccccc2)c1C(=O)Nc1ccc(-n2ccnc2)nc1OC(C)C ; Cc1onc(-c2ccccc2)c1C(=O)Nc1ccc(-c2cnc(CN)nc2)nc1OC(C)C ; Cc1onc(-c2ccc(F)cc2)c1C(=O)Nc1ccc(-c2cnc3n2CCNC3)nc1OC(C)C ; Cc1onc(-c2ccc(F)cc2)c1C(=O)Nc1ccc(-c2cnc3n2CCN(C)C3)nc1OC(C)C] 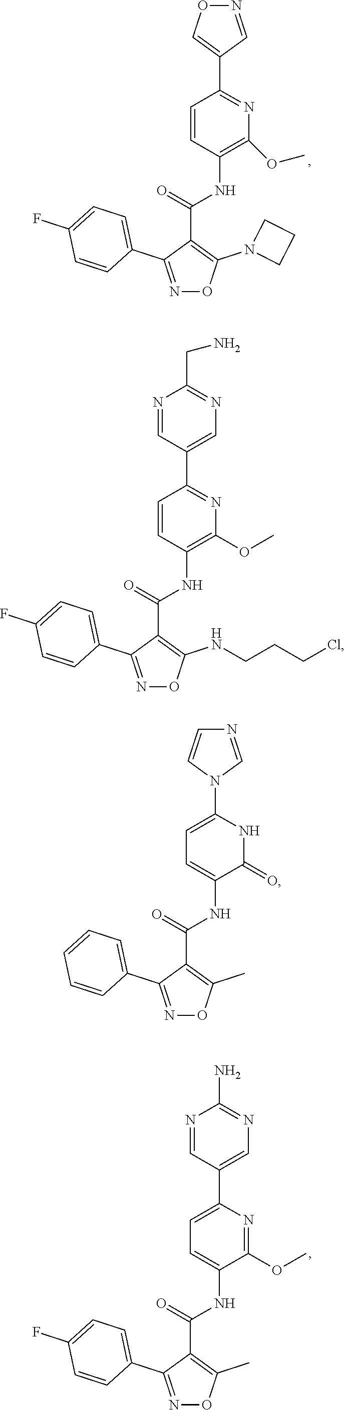 [SMILES: COc1nc(-c2cnoc2)ccc1NC(=O)c1c(-c2ccc(F)cc2)noc1N1CCC1 ; COc1nc(-c2cnc(CN)nc2)ccc1NC(=O)c1c(-c2ccc(F)cc2)noc1NCCCCl ; Cc1onc(-c2ccccc2)c1C(=O)Nc1ccc(-n2ccnc2)[nH]c1=O ; COc1nc(-c2cnc(N)nc2)ccc1NC(=O)c1c(-c2ccc(F)cc2)noc1C] 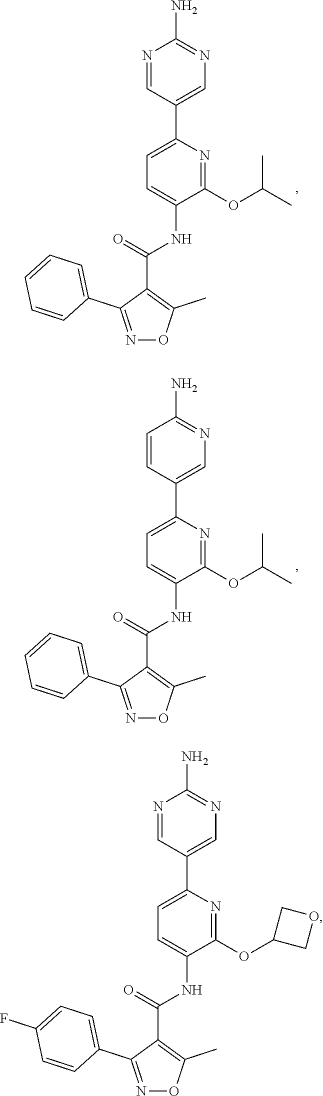 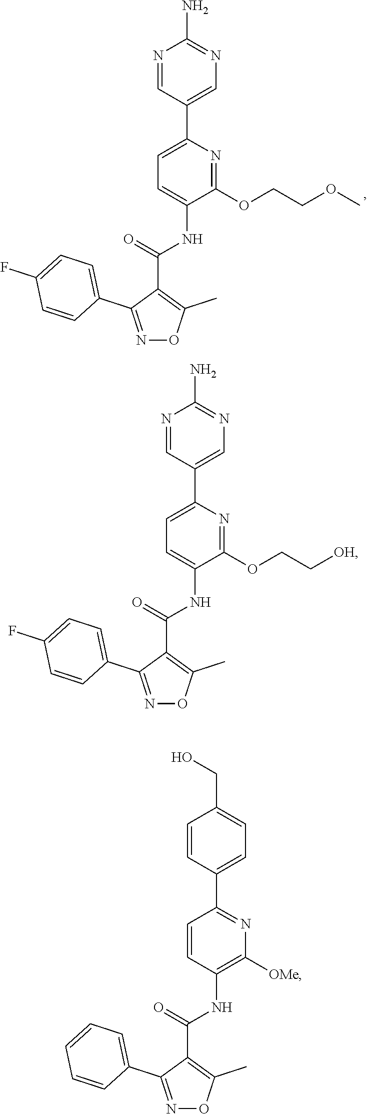 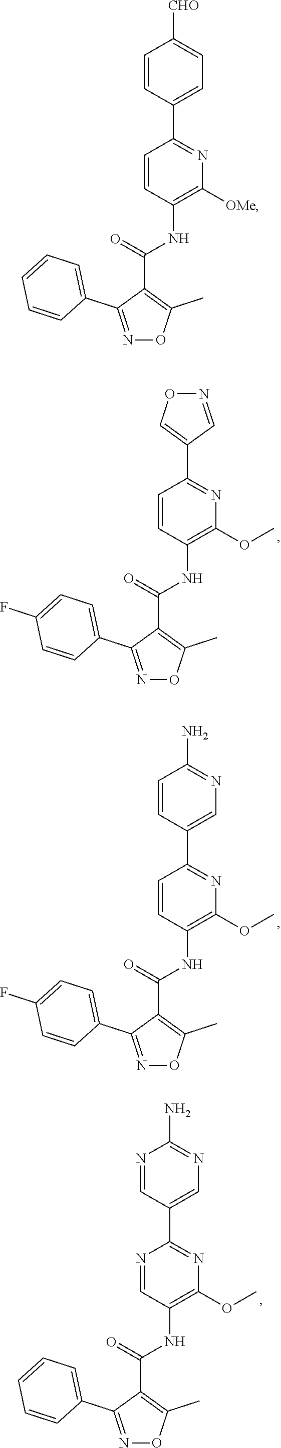 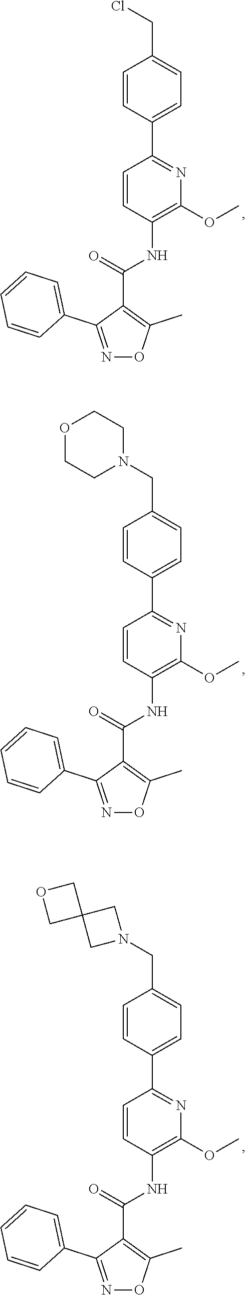 [SMILES: COc1nc(-c2ccc(CCl)cc2)ccc1NC(=O)c1c(-c2ccccc2)noc1C ; COc1nc(-c2ccc(CN3CCOCC3)cc2)ccc1NC(=O)c1c(-c2ccccc2)noc1C ; COc1nc(-c2ccc(CN3CC4(COC4)C3)cc2)ccc1NC(=O)c1c(-c2ccccc2)noc1C] 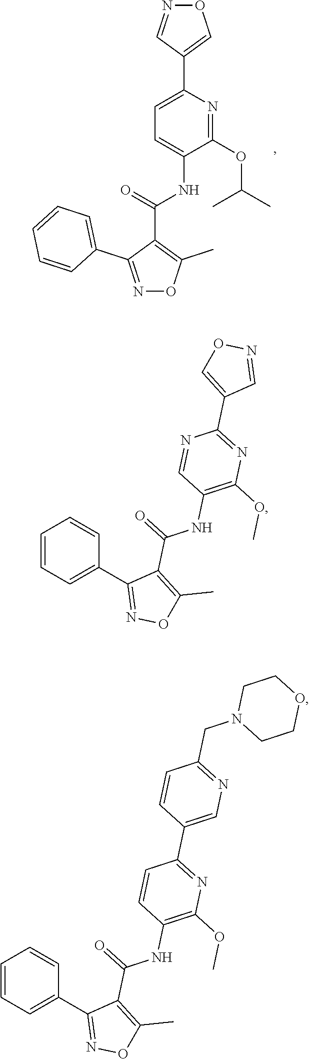 [SMILES: Cc1onc(-c2ccccc2)c1C(=O)Nc1ccc(-c2cnoc2)nc1OC(C)C ; COc1nc(-c2cnoc2)ncc1NC(=O)c1c(-c2ccccc2)noc1C ; COc1nc(-c2ccc(CN3CCOCC3)nc2)ccc1NC(=O)c1c(-c2ccccc2)noc1C] 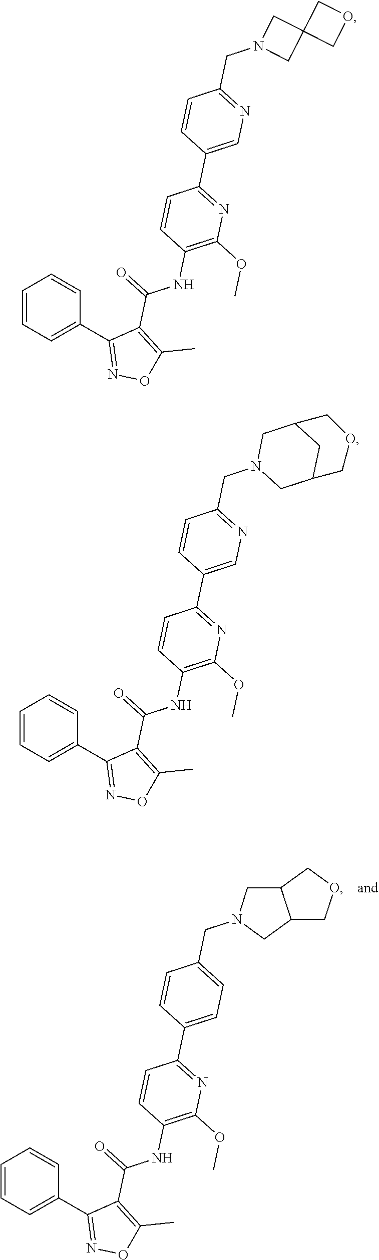 [SMILES: COc1nc(-c2ccc(CN3CC4(COC4)C3)nc2)ccc1NC(=O)c1c(-c2ccccc2)noc1C ; COc1nc(-c2ccc(CN3CC4COCC(C4)C3)nc2)ccc1NC(=O)c1c(-c2ccccc2)noc1C ; COc1nc(-c2ccc(CN3CC4COCC4C3)cc2)ccc1NC(=O)c1c(-c2ccccc2)noc1C ; "and"] 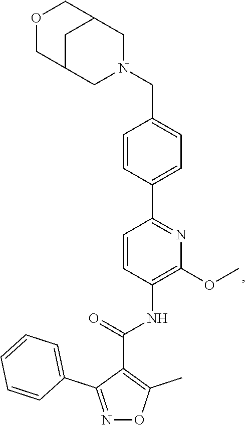 [SMILES: COc1nc(-c2ccc(CN3CC4COCC(C4)C3)cc2)ccc1NC(=O)c1c(-c2ccccc2)noc1C]
or a pharmaceutically acceptable salt thereof.

18. A pharmaceutical composition comprising the compound of claim 1, or a pharmaceutically acceptable salt thereof, and a pharmaceutically acceptable excipient.

19.-21. (canceled)

22. A method of preventing or treating a disease or medical disorder mediated by α5-GABA_{A}Rs in a subject, the method comprising administering to the subject an effective amount of the compound of claim 1, or a pharmaceutically acceptable salt thereof.

23. The method of claim 22, wherein the disease or medical disorder mediated by α5-GABA_{A}Rs is selected from: Alzheimer's disease, Parkinson's disease, Huntington's disease, cognitive dysfunction, memory deficit, age-related cognitive impairment, a bipolar disorder, autism, Down syndrome, neurofibromatosis type I, a sleep disorder, a disorder of circadian rhythms, amyotrophic lateral sclerosis (ALS), a psychotic disorder, psychosis, post-traumatic stress disorder, an anxiety disorder, a generalized anxiety disorder, a panic disorder, a delusional disorder, an obsessive/compulsive disorder, an acute stress disorder, drug addiction, alcohol disorders, drug withdrawal symptoms, a movement disorder, restless leg syndrome, a cognition deficiency disorder, multi-infarct dementia, vascular dementia, a mood disorder, depression, a neuropsychiatric condition, attention-deficit/hyperactivity disorder, neuropathic pain, chronic neuroinflammation, cognitive dysfunction associated with stroke, cognitive dysfunction associated with brain injury or trauma, cognitive dysfunction associated with a brain tumour, an attentional disorder, and Dup15q syndrome.

24. The method of claim 22, wherein the disease or medical disorder mediated by α5-GABA_{A}Rs is a neurological or neuropsychiatric disorder.

25. The method of claim 22, wherein the disease or medical disorder mediated by α5-GABA_{A}Rs are psychiatric and/or neurological symptoms caused by or associated with a viral or bacterial infection.

26. The method of claim 22, wherein the disease or medical disorder mediated by α5-GABA_{A}Rs is depression.

27. The method of claim 22, wherein the disease or medical disorder mediated by α5-GABA_{A}Rs is cognitive impairment associated with a psychotic disorder.

28. The compound according to claim 3, wherein R¹ is selected from:

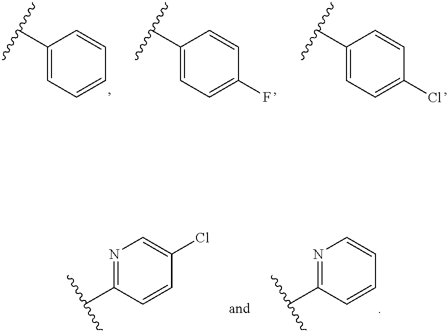 [SMILES: *c1ccccc1 ; *c1ccc(F)cc1 ; *c1ccc(Cl)cc1 ; *c1ccc(Cl)cn1 ; *c1ccccn1]

29. The compound according to claim 14, wherein
(iii) Ring B is selected from: 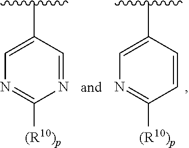
wherein p is 1; and [symbol] indicates the point of attachment to the remainder of the compound of Formula (I).

30. The compound according to claim 16, wherein Ring B is:

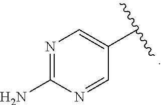 [SMILES: *c1cnc(N)nc1]

31. The method according to claim 23, wherein when the disease or medical disorder mediated by α5-GABA_{A}Rs is:
cognitive dysfunction, then the cognitive dysfunction is cognitive dysfunction associated with chemotherapy, an anaesthetic, or a bacterial infection or a viral infection,
age-related cognitive impairment, then the age-related cognitive impairment is mild cognitive impairment,
psychotic disorder, then the psychotic disorder is schizophrenia, schizoaffective disorder, schizophreniform disorder, substance-induced psychotic disorder or paraphrenia,
alcohol disorders, then the alcohol disorders are alcohol addiction.

32. The method according to claim 23, wherein the disease or medical disorder mediated by α5-GABA_{A}Rs is treatment or prevention of post-operative anaesthetic induced cognitive dysfunction.

33. The method according to claim 22, wherein the disease or medical disorder mediated by α5-GABA_{A}Rs is cognitive dysfunction associated with a neurological disorder or neuropsychiatric disorder.

34. The method according to claim 33, wherein the neurological disorder or neuropsychiatric disorder is a neurodevelopment disorder selected from attention deficit disorder (ADHD), Down Syndrome, a learning disability, cerebral palsy, autism, and a speech disorder.

35. The method according to claim 33, wherein the neurological disorder or neuropsychiatric disorder is a neurodegenerative disorder selected from Alzheimer's disease, dementia, Parkinson's disease, Huntington's disease, amyotrophic lateral sclerosis (ALS), and Creutzfeldt-Jakob disease (CJD).
